SEQUENCE LISTING (810 sequences)

SEQ ID NO: 1 - protein, 119 aa, synthetic construct
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 2 - protein, 8 aa, synthetic construct
GSIASIHA

SEQ ID NO: 3 - protein, 8 aa, synthetic construct
ITWSGGIT

SEQ ID NO: 4 - protein, 12 aa, synthetic construct
AGDKHQSSWYDY

SEQ ID NO: 5 - protein, 125 aa, synthetic construct
EVQLVESGGGLVKPGGSLRLSCAASGFTFSDESMTWMRQAPGKGLEWVSYISSGGGVKFYADSVKGRFTISRDNAKNSLYLQMNSLRAEDTAVYYCAREAPLRLGESPHDAFDISGQGTMVTVSS

SEQ ID NO: 6 - protein, 5 aa, synthetic construct
DESMT

SEQ ID NO: 7 - protein, 17 aa, synthetic construct
YISSGGGVKFYADSVKG

SEQ ID NO: 8 - protein, 16 aa, synthetic construct
EAPLRLGESPHDAFDI

SEQ ID NO: 9 - protein, 120 aa, synthetic construct
EVQLVESGGGLVQPGGSLRLSCAASGFAFSNYYMYWVRQAPGKGLEWMGGINPSNGGTNFNEKFKNRVTISRDNSKNNLYLQMNSLRAEDTALYYCARRDYRFDMGFDYWGQGTLVTVSS

SEQ ID NO: 10 - protein, 112 aa, synthetic construct
DIQMTQSPSSMSASVGDRVTFTCRASKGVSTSGYSYLHWFQQKPGKSPKTLIYLASYLESGVPSRFSGSGSGQDYTLTISSLQPEDMATYYCQHSRDLPLTFGAGTKLELKR

SEQ ID NO: 11 - protein, 120 aa, synthetic construct
EVQLVESGGGLVQPGGSLRLSCAASGFAFSSYYMYWVRQAPGKGLEWMGGVNPSNGGTNFNEKFKSRVTISRDNSKNNLYLQMNSLRAEDTALYYCARRDYRYDMGFDYWGQGTLVTVSS

SEQ ID NO: 12 - protein, 112 aa, synthetic construct
DIQMTQSPSSMSASVGDRVTFTCRASKGVSTSGYSYLHWFQQKPGKSPKTLIYLASYLESGVPSRFSGSGSGQDYTLTISSLQPEDMATYYCQHSRELPLTFGAGTKLELKR

SEQ ID NO: 13 - protein, 125 aa, synthetic construct
QVQLQESGGGSVQAGGSLRLSCVASQYTYNTVGWFRQAPGKEREGVAGIYNGGDQTYYSESAKGRFTISQDNAKRTVYLQMNSLKPEDTAMYYCAAGRLIVSGRWSMTKEEYQYWGQGTQVTVSS

SEQ ID NO: 14 - protein, 6 aa, synthetic construct
QYTYNT

SEQ ID NO: 15 - protein, 8 aa, synthetic construct
IYNGGDQT

SEQ ID NO: 16 - protein, 20 aa, synthetic construct
AAGRLIVSGRWSMTKEEYQY

SEQ ID NO: 17 - protein, 115 aa, synthetic construct
EVQLVESGGGEVQPGGSLRLSCAASGSITGANTMGWYRQAPGKQRDLVALIGNYVTHYAESVKGRFTISRDNAKNTVYLQMSSLRAEDTAVYYCYLYTDNLGTSWGQGTLVTVKP

SEQ ID NO: 18 - protein, 5 aa, synthetic construct
ANTMG

SEQ ID NO: 19 - protein, 15 aa, synthetic construct
LIGNYVTHYAESVKG

SEQ ID NO: 20 - protein, 8 aa, synthetic construct
YTDNLGTS

SEQ ID NO: 21 - protein, 5 aa, synthetic construct
GGGGS

SEQ ID NO: 22 - protein, 10 aa, synthetic construct
GGGGSGGGGS

SEQ ID NO: 23 - protein, 20 aa, synthetic construct
  VARIANT 1..20: note = This sequence may encompass 1-10 GS repeating units
GSGSGSGSGSGSGSGSGSGS

SEQ ID NO: 24 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 25 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 26 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 27 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 28 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 29 - protein, 15 aa, synthetic construct
GGGGSGGGGSGGGGS

SEQ ID NO: 30 - protein, 20 aa, synthetic construct
GGGGSGGGGSGGGGSGGGGS

SEQ ID NO: 31 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 32 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 33 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42-45 - skipped (4 entries)

SEQ ID NO: 46 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 49 - protein, 111 aa, synthetic construct [sequence omitted]

SEQ ID NO: 50 - protein, 113 aa, synthetic construct [sequence omitted]

SEQ ID NO: 51 - protein, 107 aa, synthetic construct [sequence omitted]

SEQ ID NO: 52 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 443 aa, synthetic construct
QVQLVESGGGLVKPGGSLRLSCAASGFTFSNYGMSWIRQAPGKGLEWSTISGGGSNIYYADSVKGRFTISRDNAKNSLYLQMNSLRAEDTAVYYCVSYYYGIDFWGQGTSVTVSSASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 70 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 73 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 74 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 75 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 76 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 77 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 78 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 5 aa, synthetic construct
NYYMY

SEQ ID NO: 81 - protein, 17 aa, synthetic construct
GINPSNGGTNFNEKFKN

SEQ ID NO: 82 - protein, 11 aa, synthetic construct
RDYRFDMGFDY

SEQ ID NO: 83 - protein, 15 aa, synthetic construct
RASKGVSTSGYSYLH

SEQ ID NO: 84 - protein, 7 aa, synthetic construct
LASYLES

SEQ ID NO: 85 - protein, 9 aa, synthetic construct
QHSRDLPLT

SEQ ID NO: 86 - protein, 5 aa, synthetic construct
NSGMH

SEQ ID NO: 87 - protein, 17 aa, synthetic construct
VIWYDGSKRYYADSVKG

SEQ ID NO: 88 - protein, 4 aa, synthetic construct
NDDY

SEQ ID NO: 89 - protein, 11 aa, synthetic construct
RASQSVSSYLA

SEQ ID NO: 90 - protein, 7 aa, synthetic construct
DASNRAT

SEQ ID NO: 91 - protein, 9 aa, synthetic construct
QQSSNWPRT

SEQ ID NO: 92 - protein, 9 aa, synthetic construct
FTFSNYGMS

SEQ ID NO: 93 - protein, 10 aa, synthetic construct
TISGGGSNIY

SEQ ID NO: 94 - protein, 9 aa, synthetic construct
VSYYYGIDF

SEQ ID NO: 95 - protein, 11 aa, synthetic construct
KASQDVTTAVA

SEQ ID NO: 96 - protein, 7 aa, synthetic construct
WASTRHT

SEQ ID NO: 97 - protein, 9 aa, synthetic construct
QQHYTIPWT

SEQ ID NO: 98 - protein, 8 aa, synthetic construct
GLTFSSSG

SEQ ID NO: 99 - protein, 8 aa, synthetic construct
IWYDGSKR

SEQ ID NO: 100 - protein, 6 aa, synthetic construct
ATNNDY

SEQ ID NO: 101 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 7 aa, synthetic construct
TASNRAT

SEQ ID NO: 103 - protein, 9 aa, synthetic construct
QQYSNWPRT

SEQ ID NO: 104 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 246 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 106 - protein, 448 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 218 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 251 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 109 - protein, 251 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 110 - protein, 250 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSGGGGSGGGGSGGGGSGGGGSEIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEI

SEQ ID NO: 111 - protein, 251 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKGGGGSGGGGSGGGGSGGGGSQVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 112 - protein, 250 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 113 - protein, 10 aa, synthetic construct
GYTFTSYYMY

SEQ ID NO: 114 - protein, 17 aa, synthetic construct
GVNPSNGGTNFNEKFKS

SEQ ID NO: 115 - protein, 11 aa, synthetic construct
RDYRYDMGFDY

SEQ ID NO: 116 - skipped

SEQ ID NO: 117 - protein, 6 aa, synthetic construct
LASYLE

SEQ ID NO: 118 - protein, 9 aa, synthetic construct
QHSRELPLT

SEQ ID NO: 119 - protein, 250 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 120 - protein, 317 aa, Homo sapiens
LTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 121 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 123 aa, synthetic construct [sequence omitted]

SEQ ID NO: 123 - protein, 10 aa, synthetic construct
GYTFTNYGMN

SEQ ID NO: 124 - protein, 16 aa, synthetic construct
WINTYTGEPTYAADFK

SEQ ID NO: 125 - protein, 14 aa, synthetic construct
YPHYYGSSHWYFDV

SEQ ID NO: 126 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 107 aa, synthetic construct [sequence omitted]

SEQ ID NO: 128 - protein, 11 aa, synthetic construct
SASQDISNYLN

SEQ ID NO: 129 - protein, 7 aa, synthetic construct
FTSSLHS

SEQ ID NO: 130 - protein, 9 aa, synthetic construct
QQYSTVPWT

SEQ ID NO: 131 - skipped

SEQ ID NO: 132 - protein, 120 aa, synthetic construct
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 133 - protein, 6 aa, synthetic construct
DYYYMT

SEQ ID NO: 134 - protein, 16 aa, synthetic construct
FIDPDDDPYYATWAKG

SEQ ID NO: 135 - protein, 11 aa, synthetic construct
GDHNSGWGLDI

SEQ ID NO: 136 - skipped

SEQ ID NO: 137 - protein, 111 aa, synthetic construct
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLG

SEQ ID NO: 138 - protein, 11 aa, synthetic construct
QASEIIHSWLA

SEQ ID NO: 139 - protein, 7 aa, synthetic construct
LASTLAS

SEQ ID NO: 140 - protein, 12 aa, synthetic construct
QNVYLASTNGAN

SEQ ID NO: 141 - protein, 451 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSP

SEQ ID NO: 142 - protein, 121 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTV

SEQ ID NO: 143 - protein, 5 aa, synthetic construct
HYGMN

SEQ ID NO: 144 - protein, 17 aa, synthetic construct
WINTYTGEPTYAADFKR

SEQ ID NO: 145 - protein, 5 aa, synthetic construct
NYGMN

SEQ ID NO: 146 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 107 aa, synthetic construct [sequence omitted]

SEQ ID NO: 148-149 - skipped (2 entries)

SEQ ID NO: 150 - protein, 231 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 151 - skipped

SEQ ID NO: 152 - protein, 116 aa, synthetic construct
DVQLVQSGVEVKNPGASVKVSCRASGYSFTNSGINWVKQAPGKGLKWMGWINTYTGEPTYADDFKGRFAFSLETSASSAYLQINNLKNEDTATYFCARFGDGYYWFFDVWGAGTTV

SEQ ID NO: 153 - protein, 10 aa, synthetic construct
GYSFTNSGIN

SEQ ID NO: 154 - protein, 14 aa, synthetic construct
INTYTGEPTYADDF

SEQ ID NO: 155 - protein, 10 aa, synthetic construct
FGDGYYWFFD

SEQ ID NO: 156 - skipped

SEQ ID NO: 157 - protein, 112 aa, synthetic construct
DIVMTQSPDSLAVSLGERATINCKSSQSLLNSRTRKNFLAWYQQKPGQSPKLLIYWASTRESGVPDRFTGSGSGTDFTLTISSVQAEDLAVYYCKQSYNLYTFGGGTNLEIK

SEQ ID NO: 158 - protein, 17 aa, synthetic construct
KSSQSLLNSRTRKNFLA

SEQ ID NO: 159 - protein, 7 aa, synthetic construct
WASTRES

SEQ ID NO: 160 - protein, 11 aa, synthetic construct
KQSYNLYTFGG

SEQ ID NO: 161 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 162 - protein, 450 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 730 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 164 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 165 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 166 - protein, 730 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSSGGGGSGGGGSGGGGSDVQLVESGGGLVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSS

SEQ ID NO: 167 - protein, 450 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 168 - protein, 453 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 734 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPPGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

SEQ ID NO: 170 - protein, 451 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 171 - protein, 597 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

SEQ ID NO: 172 - protein, 732 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

SEQ ID NO: 173-174 - skipped (2 entries)

SEQ ID NO: 175 - protein, 125 aa, synthetic construct
EVQLLESGGGLVQPGGSLRLSCAASGSTFSSYTVSWFRQAPGKEREFVSAIIGSSGHTYYSDSVKGRFTISRDNSKNTLYLQMNSLKPEDTAVYYCAADRVYDHEEYGITEDYAYGQGTLVTVSS

SEQ ID NO: 176 - protein, 125 aa, synthetic construct
EVQLLESGGGLVQPGGSLRLSCAASGFTFSSYFIGWFRQAPGKEREFVSAIIGSGGSTYYDDAVKGRFTISRDNSKNTLYLQMNSLKPEDTAVYYCAVKRHAYYGRDRSYNYDYWGQGTLVTVSS

SEQ ID NO: 177 - protein, 121 aa, synthetic construct
EVQLLESGGGLVQPGGSLRLSCAASGGTFSIYGIGWFRQAPGKEREFVSGIILTGGSTYYADSVKGRFTISRDNSKNTLYLQMNSLKPEDTAVYYCYVDVPLYDTGYHTYWGQGTLVTVSS

SEQ ID NO: 178 - protein, 120 aa, synthetic construct
EVQLLESGGGLVQPGGSLRLSCAASGSSFSSYDLGWFRQAPGKEREFVSAYILSGGSTYYEDSVKGRFTISRDNSKNTLYLQMNSLKPEDTAVYYCYADYTVERDRYSYWGQGTLVTVSS

SEQ ID NO: 179 - protein, 118 aa, synthetic construct
EVQLLESGGGLVQPGGSLRLSCAASGFIFSSYHLAWFRQAPGKEREFVSASIGSSEDTEYADSVKGRFTISRDNSKNTLYLQMNSLKPEDTAVYYCYADHGVKYYEYWGRGTLVTVSS

SEQ ID NO: 180 - protein, 120 aa, synthetic construct
EVQLLESGGGLVQPGGSLRLSCAASGRTYEIYGMGWFRQAPGKEREFVSGSISSEGSTFYADSVKGRFTISRDNSKNTLYLQMNSLKPEDTAVYYCYADQSYPYGYTTYWGQGTLVTVSS

SEQ ID NO: 181 - protein, 117 aa, synthetic construct
EVQLLESGGGLVQPGGSLRLSCAASGSSFSIYDMGWFRQAPGKEREFVSAYITSGGDTYYEDSVKGRFTISRDNSKNTLYLQMNSLKPEDTAVYYCYAGVPQDEDYWGQGTLVTVSS

SEQ ID NO: 182 - protein, 119 aa, synthetic construct
EVQLLESGGGLVQPGGSLRLSCAASGRIFSILDVGWFRQAPGKEREFVSAISGSSGGRYYADSVKGRFTISRDNSKNTLYLQMNSLKPEDTAVYYCSVLESEYQLYDYWGQGTLVTVSS

SEQ ID NO: 183 - protein, 117 aa, synthetic construct
EVQLLESGGGLVQPGGSLRLSCAASGRTLSSYDMGWFRQAPGKEREFVSAFITSGGSTFYEDSVKGRFTISRDNSKNTLYLQMNSLKPEDTAVYYCYADYVHDRGYWGQGTLVTVSS

SEQ ID NO: 184 - protein, 117 aa, synthetic construct
EVQLLESGGGLVQPGGSLRLSCAASGSTFSSYTMAWFRQAPGKEREFVSANITSGGDTYYADSVKGRFTISRDNSKNTLYLQMNSLKPEDTAVYYCAADVVYDEDYWGLSTHYTVSS

SEQ ID NO: 185 - protein, 117 aa, synthetic construct
EVQLLESGGGLVQPGGSLRLSCAASGFEFSIYDLGWFRQAPGEEREFVSAYITSEGSTYYVDSVKGRFTISRDNSKNTLYLQMNSLKPEDTAVYYCYADRGLHYSYWGQGTLVTVSS

SEQ ID NO: 186 - protein, 118 aa, synthetic construct
EVQLLESGGGLVQPGGSLRLSCAASGIIFSIYDMGWFRQAPGKEREFVSAFITSGGSTYYPDSVKGRFTISRDNSKNTLYLQMNSLKPEDTAVYYCYAEEGVGLEGYWGQGTLVTVSS

SEQ ID NO: 187 - protein, 120 aa, synthetic construct
EVQLLESGGGLVQPGGSLRLSCAASGSPEREYGVGWFRQAPGKEREFVSGVISSGGSTYYADSVKGRFTISRDNSKNTLYLQMNSLKPEDTAVYYCYADHGTDQGSYTYWGQGTLVTVSS

SEQ ID NO: 188 - protein, 118 aa, synthetic construct
EVQLLESGGGLVQPGGSLRLSCAASGSFFSSYSVGWFRQAPGKEREFVSAIVIRTGSTYYGDSVKGRFTISRDNSKNTLYLQMNSLKPEDTAVYYCYADESFDQESNWGQGTLVTVSS

SEQ ID NO: 189 - protein, 119 aa, synthetic construct
EVQLLESGGGLVQPGGSLRLSCAASGSSFSSYIMGWFRQAPGKEREFVSAVIGSGGDTYYADSVKGRFTISRDNSKNTLYLQMNSLKPEDTAVYYCYVDADTPVTTSHWGQGTLVTVSS

SEQ ID NO: 190 - protein, 119 aa, synthetic construct
EVQLLESGGGLVQPGGSLRLSCAASGSIFSSYAMGWFRQAPGKEREFVSAIVGSSGSTYYADSVKGRFTISRDNSKNTLYLQMNSLKPEDTAVYYCAVQRGIDYSFDFWGQGTLVTVSS

SEQ ID NO: 191 - protein, 118 aa, synthetic construct
EVQLLESGGGLVQPGGSLRLSCAASGSIFSIYAMGWFRQAPGKEREFVSASVISSGSTYYADSVKGRFTISRDNSKNTLYLQMNSLKPEDTAVYYCSAHTDYSLYDYWGQGTLVTVSS

SEQ ID NO: 192 - protein, 118 aa, synthetic construct
EVQLLESGGGLVQPGGSLRLSCAASGFIFSFYHLAWFRQAPGKEREFVSASIGSSEDTEYADSVKGRFTISRDNSKNTLYLQMNSLKPEDTAVYYCYAVHGVKYYEYWGQGTLVTVSS

SEQ ID NO: 193 - protein, 117 aa, synthetic construct
EVQLLESGGGLVQPGGSLRLSCAASGSIFEFYAVGWFRQAPGKEREFVSATISSTGSKYYEDSVKGRFTISRDNSKNTLYLQMNSLKPEDTAVYYCAVDIGEGFDYWGQGTLVTVSS

SEQ ID NO: 194 - protein, 118 aa, synthetic construct
EVQLLESGGGLVQPGGSLRLSCAASGSIFSYYGMGWFRQAPGKEREFVSGIISSGGSTYYADSVKGRFTISRDNSKNTLYLQMNSLKPEDTAVYYCYADTYTQRYTYWGQGTLVTVSS

SEQ ID NO: 195 - protein, 118 aa, synthetic construct
EVQLLESGGGLVQPGGSLRLSCAASGSIFSSYAVGWFRQAPGKEREFVSAQIISSGSTYYEDSVKGRFTISRDNSKNTLYLQMNSLKPEDTAVYYCAAEEYYGYFDYWGQGTLVTVSS

SEQ ID NO: 196 - protein, 121 aa, synthetic construct
EVQLLESGGGLVQPGGSLRLSCAASGSIFSIYAMSWFRQAPGKEREFVSALIGSGGSTFYADSVKGRFTISRDNSKNTLYLQMNSLKPEDTAVYYCYATVGDHPQFQYDYWGQGTLVTVSS

SEQ ID NO: 197 - protein, 124 aa, synthetic construct
EVQLLESGGGLVQPGGSLRLSCAASGRIFSRYAMGWFRQAPGKEREFVSAYIATSGSRYYGDSVKGRFTISRDNSKNTLYLQMNSLKPEDTAVYYCGVSLPQGTGQGYDLYDTWGQGTLVTVSS

SEQ ID NO: 198 - protein, 117 aa, synthetic construct
EVQLLESGGGLVQPGGSLRLSCAASGFEFSIYHLGWFRQAPGEEREFVSAYITSEGSTYYVDSVKGRFTISRDNSKNTLYLQMNSLKPEDTAVYYCYADRGLHYSYWGQGTLVTVSS

SEQ ID NO: 199 - skipped

SEQ ID NO: 200 - protein, 127 aa, synthetic construct
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

SEQ ID NO: 201 - protein, 116 aa, synthetic construct
EVQLLVSGGGLVDPGGSLRLSCAASGFTFKAYPMMWVRQAPGKGLEWVSEISPSGSYTYYADSVRGRFFISRDNSKNTLYLQMNSLRAEDTAVYYCAKDPRKLDYWGQGTLWTVSS

SEQ ID NO: 202 - protein, 5 aa, synthetic construct
AYPMM

SEQ ID NO: 203 - protein, 17 aa, synthetic construct
EISPSGSYTYYADSVRG

SEQ ID NO: 204 - protein, 7 aa, synthetic construct
DPRKLDY

SEQ ID NO: 205 - protein, 116 aa, synthetic construct
EVQLLESGGGLVQPGGSLRLSCAASGFTFHLYDMMWVRQAPGKGLEWVSFIGGDGLNTYYADSVKGRFTISRDNSKNTLYLQMNSLRAEDTAVYYCAKAGTQFDYWGQGTLVTVSS

SEQ ID NO: 206 - protein, 5 aa, synthetic construct
LYDMM

SEQ ID NO: 207 - protein, 17 aa, synthetic construct
FIGGDGLNTYYADSVKG

SEQ ID NO: 208 - protein, 7 aa, synthetic construct
AGTQFDY

SEQ ID NO: 209 - protein, 124 aa, synthetic construct
EVQLLESGGGLVQPGGSLRLSCAASGFTFQWYPMWWVRQAPGKGLEWVSLIEGQGDRTYYADSVKGRFTISRDNSKNTLYLQMNSLRAEDTAVYYCAKAGDRTAGSRGNSFDYWGQGTLVTVSS

SEQ ID NO: 210 - protein, 5 aa, synthetic construct
WYPMW

SEQ ID NO: 211 - protein, 17 aa, synthetic construct
LIEGQGDRTYYADSVKG

SEQ ID NO: 212 - protein, 15 aa, synthetic construct
AGDRTAGSRGNSFDY

SEQ ID NO: 213 - protein, 116 aa, synthetic construct
EVQLLESGGGLVQPGGSLRLSCAASGFTFGAYPMMWVRQAPGKGLEWVSEISPSGSYTYYADSVKGRFTISRDNSKNTLYLQMNSLRAEDTAVYYCAKDPRKFDYWGQGTLVTVSS

SEQ ID NO: 214 - protein, 125 aa, synthetic construct
EVQLVESGGGLVQPGGSLRLSCAASGRTFSSYSMGWFRQAPGKGLEFVVAISKGGYKYDAVSVKGRFTISRDNAKNSVYLQMNSLRAEDTAVYYCASSRAYGSSRLRLADTYEYWGQGTLVTVSS

SEQ ID NO: 215 - protein, 17 aa, synthetic construct
EISPSGSYTYYADSVKG

SEQ ID NO: 216 - protein, 7 aa, synthetic construct
DPRKFDY

SEQ ID NO: 217 - protein, 125 aa, synthetic construct
DVQLVESGGGLVQPGGSLRLSCAASGRTFSSYSMGWFRQAPGKEREFVVAISKGGYKYDAVSLEGRFTISRDNAKNTVYLQMNSLRPEDTAVYYCASSRAYGSSRLRLADTYEYWGQGTLVTVSS

SEQ ID NO: 218 - protein, 5 aa, synthetic construct
SYSMG

SEQ ID NO: 219 - protein, 16 aa, synthetic construct
AISKGGYKYDAVSLEG

SEQ ID NO: 220 - protein, 17 aa, synthetic construct
SRAYGSSRLRLADTYEY

SEQ ID NO: 221 - protein, 125 aa, synthetic construct
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 222 - protein, 108 aa, synthetic construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRR

SEQ ID NO: 223 - protein, 11 aa, synthetic construct
RASQWIGPELS

SEQ ID NO: 224 - protein, 7 aa, synthetic construct
HTSILQS

SEQ ID NO: 225 - protein, 9 aa, synthetic construct
QQYMFQPRT

SEQ ID NO: 226 - protein, 150 aa, synthetic construct
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 227 - protein, 8 aa, synthetic construct
APELLGGP

SEQ ID NO: 228 - protein, 8 aa, synthetic construct
APEAAGGP

SEQ ID NO: 229 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 232 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 234 - protein, 209 aa, synthetic construct
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 8 aa, synthetic construct
APEFLGGP

SEQ ID NO: 236 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 237 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 238 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 239 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 240 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNRFTQKSLSLSLGK

SEQ ID NO: 241 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 242 - protein, 719 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSGGGGSGGGGSGGGGSGGGGSDIQMTQSPSSMSASVGDRVTFTCRASQDINTYLSWFQQKPGKSPKTLIYRANRLVSGVPSRFSGSGSGQDYTLTISSLQPEDMATYYCLQYDEFPLTFGAGTKLELKR

SEQ ID NO: 243 - protein, 718 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 244 - protein, 718 aa, synthetic construct
EVQLVESGGGLVQPGGSLRLSCAASGFAFSSYDMSWVRQAPGKGLDWVATISGGGRYTYYPDSVKGRFTISRDNSKNNLYLQMNSLRAEDTALYYCANRYGEAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 245 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIK

SEQ ID NO: 246 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKGGGGSGGGGSGGGGSGGGGSQVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSS

SEQ ID NO: 247 - protein, 720 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 248 - protein, 720 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 249 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSGGGGSGGGGSGGGGSGGGGSEIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 250 - protein, 724 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIKGGGGSGGGGSGGGGSGGGGSQVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 251 - protein, 720 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 252 - protein, 720 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKGGGGSGGGGSGGGGSGGGGSEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSS

SEQ ID NO: 253 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSNYYMYWVRQAPGKGLEWMGGINPSNGGTNFNEKFKNRVTISRDNSKNNLYLQMNSLRAEDTALYYCARRDYRFDMGFDYWGQGTLVTVSSGGGGSGGGGSGGGGSGGGGSDIQMTQSPSSMSASVGDRVTFTCRASKGVSTSGYSYLHWFQQKPGKSPKTLIYLASYLESGVPSRFSGSGSGQDYTLTISSLQPEDMATYYCQHSRDLPLTFGAGTKLELKR

SEQ ID NO: 254 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSMSASVGDRVTFTCRASKGVSTSGYSYLHWFQQKPGKSPKTLIYLASYLESGVPSRFSGSGSGQDYTLTISSLQPEDMATYYCQHSRDLPLTFGAGTKLELKRGGGGSGGGGSGGGGSGGGGSEVQLVESGGGLVQPGGSLRLSCAASGFAFSNYYMYWVRQAPGKGLEWMGGINPSNGGTNFNEKFKNRVTISRDNSKNNLYLQMNSLRAEDTALYYCARRDYRFDMGFDYWGQGTLVTVSS

SEQ ID NO: 255 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFAFSSYYMYWVRQAPGKGLEWMGGVNPSNGGTNFNEKFKSRVTISRDNSKNNLYLQMNSLRAEDTALYYCARRDYRYDMGFDYWGQGTLVTVSSGGGGSGGGGSGGGGSGGGGSDIQMTQSPSSMSASVGDRVTFTCRASKGVSTSGYSYLHWFQQKPGKSPKTLIYLASYLESGVPSRFSGSGSGQDYTLTISSLQPEDMATYYCQHSRELPLTFGAGTKLELKR

SEQ ID NO: 256 - protein, 725 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDIQMTQSPSSMSASVGDRVTFTCRASKGVSTSGYSYLHWFQQKPGKSPKTLIYLASYLESGVPSRFSGSGSGQDYTLTISSLQPEDMATYYCQHSRELPLTFGAGTKLELKRGGGGSGGGGSGGGGSGGGGSEVQLVESGGGLVQPGGSLRLSCAASGFAFSSYYMYWVRQAPGKGLEWMGGVNPSNGGTNFNEKFKSRVTISRDNSKNNLYLQMNSLRAEDTALYYCARRDYRYDMGFDYWGQGTLVTVSS

SEQ ID NO: 257 - protein, 595 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVESGGGLVQPGGSLRLSCAVSGNIYNRNFMGWFRQAPGKGLEGVSAIYTGTSRTYYADSVKGRFTISRDNSKNTVYLQMNSLRAEDTAVYYCAADLREGFWDTGVWNTWGQGTLVTVSS

SEQ ID NO: 258 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 259 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGVVQPGGSLRLSCAASGRTFSSYSMGWFRQAPGKGLEFVVAISKGGYKYDAVSLEGRFTISRDNSKNTVYLQMNSLRAEDTAVYYCASSRAYGSSRLRLADTYEYWGQGTLVTVSS

SEQ ID NO: 260 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGVVQPGGSLRLSCAASGRTFSSYSMGWFRQAPGKEREFVVAISKGGYKYDAVSLEGRFTISRDNSKNTVYLQMNSLRAEDTAVYYCASSRAYGSSRLRLADTYEYWGQGTLVTVSS

SEQ ID NO: 261 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGRTFSSYSMGWFRQAPGKEREFVVAISKGGYKYDAVSVKGRFTISRDNSKNTVYLQMGSLRAEDMAVYYCASSRAYGSSRLRLADTYEYWGQGTLVTVSS

SEQ ID NO: 262 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGRTFSSYSMGWFRQAPGKGLEFVVAISKGGYKYDAVSVKGRFTISRDNSKNTVYLQMGSLRAEDMAVYYCASSRAYGSSRLRLADTYEYWGQGTLVTVSS

SEQ ID NO: 263 - protein, 595 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGRTFSSYSMGWFRQAPGKEREFVVAISKGGYKYDAVSVKGRFTISRDNAKNTVYLQMNSLRAEDTAVYYCASSRAYGSSRLRLADTYEYWGQGTLVTVSS

SEQ ID NO: 264 - protein, 588 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGEVQPGGSLRLSCAASGSITGANTMGWYRQAPGKQRDLVALIGNYVTHYAESVKGRFTISRDNAKNTVYLQMSSLRAEDTAVYYCYLYTDNLGTSWGQGTLVTVKP

SEQ ID NO: 265 - protein, 18 aa, synthetic construct
DKTHTCPPCPAPELLGGP

SEQ ID NO: 266 - protein, 18 aa, synthetic construct
DKTHTCPPCPAPEAAGGP

SEQ ID NO: 267 - protein, 23 aa, synthetic construct
EPKSCDKTHTCPPCPAPELLGGP

SEQ ID NO: 268 - protein, 23 aa, synthetic construct
EPKSCDKTHTCPPCPAPEAAGGP

SEQ ID NO: 269 - protein, 15 aa, synthetic construct
EPKSCDKTHTCPPCP

SEQ ID NO: 270 - protein, 20 aa, synthetic construct
ESKYGPPCPSCPAPEFLGGP

SEQ ID NO: 271 - protein, 12 aa, synthetic construct
ESKYGPPCPSCP

SEQ ID NO: 272 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 273 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 274 - protein, 98 aa, synthetic construct
ASKYGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 275 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 276 - protein, 107 aa, synthetic construct [sequence omitted]

SEQ ID NO: 277 - protein, 107 aa, synthetic construct
RTVAAPSVFIFPPSDEQLKSGTASVVCLLNNFYPREAKVQWKVDNAKQSGNSQESVTEQDSKDSTYSLSSTLTLSKADYEKHKVYACEVTHQGLSSPVTKSFNRGEC

SEQ ID NO: 278 - protein, 107 aa, synthetic construct
RTVAAPSVFIFPPSDEQLKSGTASVVCLLNNFYPREAKVQWKVDNAKQSGNSQESVTEQDSKDSTYSLSSTLTLSKADYEKHKVYACEVTHQGKSSPVTKSFNRGEC

SEQ ID NO: 279 - protein, 16 aa, synthetic construct
AISKGGYKYDAVSVKG

SEQ ID NO: 280 - protein, 125 aa, synthetic construct
EVQLVESGGGVVQPGGSLRLSCAASGRTFSSYSMGWFRQAPGKGLEFVVAISKGGYKYDAVSLEGRFTISRDNSKNTVYLQMNSLRAEDTAVYYCASSRAYGSSRLRLADTYEYWGQGTLVTVSS

SEQ ID NO: 281 - protein, 125 aa, synthetic construct
EVQLVESGGGVVQPGGSLRLSCAASGRTFSSYSMGWFRQAPGKEREFVVAISKGGYKYDAVSLEGRFTISRDNSKNTVYLQMNSLRAEDTAVYYCASSRAYGSSRLRLADTYEYWGQGTLVTVSS

SEQ ID NO: 282 - protein, 125 aa, synthetic construct
EVQLVESGGGLVQPGGSLRLSCAASGRTFSSYSMGWFRQAPGKEREFVVAISKGGYKYDAVSVKGRFTISRDNSKNTVYLQMGSLRAEDMAVYYCASSRAYGSSRLRLADTYEYWGQGTLVTVSS

SEQ ID NO: 283 - protein, 125 aa, synthetic construct
EVQLVESGGGLVQPGGSLRLSCAASGRTFSSYSMGWFRQAPGKGLEFVVAISKGGYKYDAVSVKGRFTISRDNSKNTVYLQMGSLRAEDMAVYYCASSRAYGSSRLRLADTYEYWGQGTLVTVSS

SEQ ID NO: 284 - protein, 125 aa, synthetic construct
EVQLVESGGGLVQPGGSLRLSCAASGRTFSSYSMGWFRQAPGKEREFVVAISKGGYKYDAVSVKGRFTISRDNAKNTVYLQMNSLRAEDTAVYYCASSRAYGSSRLRLADTYEYWGQGTLVTVSS

SEQ ID NO: 285 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNAKQSGNSQESVTEQDSKDSTYSLSSTLTLSKADYEKHKVYACEVTHQGLSSPVTKSFNRGEC

SEQ ID NO: 286 - protein, 214 aa, synthetic construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNAKQSGNSQESVTEQDSKDSTYSLSSTLTLSKADYEKHKVYACEVTHQGKSSPVTKSFNRGEC

SEQ ID NO: 287 - protein, 123 aa, synthetic construct
EVQLVESGGGLVQPGGSLRLSCAVSGNIYNRNFMGWFRQAPGKGLEGVSAIYTGTSRTYYADSVKGRFTISRDNSKNTVYLQMNSLRAEDTAVYYCAADLREGFWDTGVWNTWGQGTLVTVSS

SEQ ID NO: 288 - protein, 5 aa, synthetic construct
RNFMG

SEQ ID NO: 289 - protein, 17 aa, synthetic construct
AIYTGTSRTYYADSVKG

SEQ ID NO: 290 - protein, 14 aa, synthetic construct
DLREGFWDTGVWNT

SEQ ID NO: 291 - protein, 592 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 292 - protein, 596 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVESGGGLVKPGGSLRLSCAASGFTFSDESMTWMRQAPGKGLEWVSYISSGGGVKFYADSVKGRFTISRDNAKNSLYLQMNSLRAEDTAVYYCAREAPLRLGESPHDAFDISGQGTMVTVSS

SEQ ID NO: 293 - protein, 598 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQVQLQESGGGSVQAGGSLRLSCVASQYTYNTVGWFRQAPGKEREGVAGIYNGGDQTYYSESAKGRFTISQDNAKRTVYLQMNSLKPEDTAMYYCAAGRLIVSGRWSMTKEEYQYWGQGTQVTVSS

SEQ ID NO: 294-298 - skipped (5 entries)

SEQ ID NO: 299 - protein, 8 aa, synthetic construct
GGLLQGPP

SEQ ID NO: 300 - protein, 123 aa, synthetic construct
EVQLVESGGGLVQPGGSLRLSCAASGISFSHYGMAWYRQAPGKGRELVAGIGLSGGSTSYADSVKGRFTISRDNAKNTLYLQMNSLRPEDTAVYYCARGHDFWSGLTPDYWGQGTLVTVSSPP

SEQ ID NO: 301 - protein, 8 aa, synthetic construct
GISFSHYG

SEQ ID NO: 302 - protein, 8 aa, synthetic construct
IGLSGGST

SEQ ID NO: 303 - protein, 14 aa, synthetic construct
ARGHDFWSGLTPDY

SEQ ID NO: 304 - protein, 127 aa, synthetic construct
EVQLVESGGGLVQPGGSLRLSCAASGFTVSSGGMGWFRQAPGKGRELVAAISPSGVTTYYPDSVEGRFTISRDNAKRMVYLQMNSLRAEDTAVYYCAKTGGSSGSWFPDDAFDVWGQGTMVTVSSPP

SEQ ID NO: 305 - protein, 8 aa, synthetic construct
GFTVSSGG

SEQ ID NO: 306 - protein, 8 aa, synthetic construct
ISPSGVTT

SEQ ID NO: 307 - protein, 18 aa, synthetic construct
AKTGGSSGSWFPDDAFDV

SEQ ID NO: 308 - protein, 126 aa, synthetic construct
EVQLVESGGGLVQPGGSLRLSCAASGFPSSSHGMAWYRQAPGKGRELVAGMSGTGATTSYADSVKGRFTISRDNAKNTLYLQMNSLRPEDTAVYYCANRWWYCSGGSCYDVDYWGQGTLVTVSSPP

SEQ ID NO: 309 - protein, 8 aa, synthetic construct
GFPSSSHG

SEQ ID NO: 310 - protein, 8 aa, synthetic construct
MSGTGATT

SEQ ID NO: 311 - protein, 17 aa, synthetic construct
ANRWWYCSGGSCYDVDY

SEQ ID NO: 312 - protein, 124 aa, synthetic construct
EVQLVESGGGLVQPGGSLRLSCAASGFTFGVYDMGWLRQAPGKEREFVAAISATGGSTYYADSVKGRFTISRDNSKNTLYLQMNSLRPEDTAVYYCATVFCSGGSCYPFGYWGQGTLVTVSSPP

SEQ ID NO: 313 - protein, 8 aa, synthetic construct
GFTFGVYD

SEQ ID NO: 314 - protein, 8 aa, synthetic construct
ISATGGST

SEQ ID NO: 315 - protein, 15 aa, synthetic construct
ATVFCSGGSCYPFGY

SEQ ID NO: 316 - protein, 125 aa, synthetic construct
DVQLVESGGGLVQPGGSLRLSCAASGLTFSTSEMGWFRQAPGKEREFVAGITGSGAFTYYVDSLKGRFTISRDNSKNTVYLQMNSLRPEDTAVYYCARDRCSSSNCVDDFDIWGPGTMVTVSSPP

SEQ ID NO: 317 - protein, 8 aa, synthetic construct
GLTFSTSE

SEQ ID NO: 318 - protein, 8 aa, synthetic construct
ITGSGAFT

SEQ ID NO: 319 - protein, 16 aa, synthetic construct
ARDRCSSSNCVDDFDI

SEQ ID NO: 320 - protein, 123 aa, synthetic construct
EVQLVESGGGLVQPGGSLRLSCAASGFTGSSTYMAWYRQAPGKGRELVAGISGNADYTSYADSVKGRFTISRDNAKNTLYLQMNSLRPEDTAVYYCARGLYSYDSSPFDYWGQGTLVTVSSPP

SEQ ID NO: 321 - protein, 8 aa, synthetic construct
GFTGSSTY

SEQ ID NO: 322 - protein, 8 aa, synthetic construct
ISGNADYT

SEQ ID NO: 323 - protein, 14 aa, synthetic construct
ARGLYSYDSSPFDY

SEQ ID NO: 324 - protein, 127 aa, synthetic construct
EVQLVESGGGLVQPGGSLRLSCAASGFTVSHYGMAWYRQAPGKGRELVAGISGTGAYTSYADSVKGRFTISKDNAKNTLYLQMNSLRPEDTAVYYCARGVLGPYGDYPYWYFDLWGRGTLVTVSSPP

SEQ ID NO: 325 - protein, 8 aa, synthetic construct
GFTVSHYG

SEQ ID NO: 326 - protein, 8 aa, synthetic construct
ISGTGAYT

SEQ ID NO: 327 - protein, 18 aa, synthetic construct
ARGVLGPYGDYPYWYFDL

SEQ ID NO: 328 - protein, 124 aa, synthetic construct
EVQLVESGGGLVQPGGSLRLSCAASGFSVSGHGMAWYRQAPGKGRELVAGISSGGTVTSYADSVKGRFTISRDNAKNTLYLQMNSLRPEDTAVYYCARSWYCSGGSCYPDYWGQGTLVTVSSPP

SEQ ID NO: 329 - protein, 8 aa, synthetic construct
GFSVSGHG

SEQ ID NO: 330 - protein, 8 aa, synthetic construct
ISSGGTVT

SEQ ID NO: 331 - protein, 15 aa, synthetic construct
ARSWYCSGGSCYPDY

SEQ ID NO: 332 - protein, 122 aa, synthetic construct
DVQLVESGGGLVQPGGSLRLSCAASGFSFTKYGMGWFRQAPGKEREFVAGVTGSGDATYYVDSLKGRFTISRDNSKNTVYLQMNSLRPEDTAVYYCAREAVAGPYYFDYWGQGTLVTVSSPP

SEQ ID NO: 333 - protein, 8 aa, synthetic construct
GFSFTKYG

SEQ ID NO: 334 - protein, 8 aa, synthetic construct
VTGSGDAT

SEQ ID NO: 335 - protein, 13 aa, synthetic construct
AREAVAGPYYFDY

SEQ ID NO: 336 - protein, 122 aa, synthetic construct
EVQLVESGGGLVQPGGSLRLSCAASGFDFSTYPMGWLRQAPGKEREFVAAISTSGGVTYYADSVKGRFTISRDNSKNTLYLQMNSLRPEDTAVYYCAREARGNYAHFDYWGQGTLVTVSSPP

SEQ ID NO: 337 - protein, 8 aa, synthetic construct
GFDFSTYP

SEQ ID NO: 338 - protein, 8 aa, synthetic construct
ISTSGGVT

SEQ ID NO: 339 - protein, 13 aa, synthetic construct
AREARGNYAHFDY

SEQ ID NO: 340 - protein, 123 aa, synthetic construct
EVQLVESGGGLVQPGGSLRLSCAASGFGFSGSAMGWLRQAPGKEREFVAAISAGSTTTYYADSVKGRFTISRDNSKNTLYLQMNSLRPEDTAVYYCAKGVDYYGPLPFDYWGQGTLVTVSSPP

SEQ ID NO: 341 - protein, 8 aa, synthetic construct
GFGFSGSA

SEQ ID NO: 342 - protein, 8 aa, synthetic construct
ISAGSTTT

SEQ ID NO: 343 - protein, 14 aa, synthetic construct
AKGVDYYGPLPFDY

SEQ ID NO: 344 - protein, 120 aa, synthetic construct
EVQLVESGGGLVQPGGSLRLSCAASGFSFGSYGMGWLRQAPGKEREFVAAISSTGGVTYYADSVKGRFTISRDNSKNTLYLQMNSLRPEDTAVYYCARIYGPWYFDLWGRGTLVTVSSPP

SEQ ID NO: 345 - protein, 8 aa, synthetic construct
GFSFGSYG

SEQ ID NO: 346 - protein, 8 aa, synthetic construct
ISSTGGVT

SEQ ID NO: 347 - protein, 11 aa, synthetic construct
ARIYGPWYFDL

SEQ ID NO: 348 - protein, 122 aa, synthetic construct
DVQLVESGGGLVQPGGSLRLSCAASGFSFGDHEMGWFRQAPGKEREFVAGITGTGEYTYYVDSLKGRFTISRDNSKNTVYLQMNSLRPEDTAVYYCARVGYSSSSYFDYWGQGTLVTVSSPP

SEQ ID NO: 349 - protein, 8 aa, synthetic construct
GFSFGDHE

SEQ ID NO: 350 - protein, 8 aa, synthetic construct
ITGTGEYT

SEQ ID NO: 351 - protein, 13 aa, synthetic construct
ARVGYSSSSYFDY

SEQ ID NO: 352 - protein, 122 aa, synthetic construct
DVQLVESGGGLVQPGGSLRLSCAASGFTFSDAEMGWFRQAPGKEREFVAGIGGSVSSTYYVDSLKGRFTISRDNSKNTVYLQMNSLRPEDTAVYYCARAAVAGPYYFDYWGQGTLVTVSSPP

SEQ ID NO: 353 - protein, 8 aa, synthetic construct
GFTFSDAE

SEQ ID NO: 354 - protein, 8 aa, synthetic construct
IGGSVSST

SEQ ID NO: 355 - protein, 13 aa, synthetic construct
ARAAVAGPYYFDY

SEQ ID NO: 356 - protein, 118 aa, synthetic construct
EVQLVESGGGLVQPGGSLRLSCAASGIAFSNHAMGWFRQAPGKGRELVAAIIGSGAGTYYPDSVEGRFTISRDNAKRMVYLQMNSLRAEDTAVYYCTTAAATNDYWGQGTLVTVSSPP

SEQ ID NO: 357 - protein, 8 aa, synthetic construct
GIAFSNHA

SEQ ID NO: 358 - protein, 8 aa, synthetic construct
IIGSGAGT

SEQ ID NO: 359 - protein, 9 aa, synthetic construct
TTAAATNDY

SEQ ID NO: 360 - protein, 123 aa, synthetic construct
EVQLVESGGGLVQPGGSLRLSCAASGFGFSTYDMAWYRQAPGKGRELVAGISTSGTWTSYADSVKGRFTISRDNAKNTLYLQMNSLRPEDTAVYYCASLIYGDYEAGLDYWGQGTLVTVSSPP

SEQ ID NO: 361 - protein, 8 aa, synthetic construct
GFGFSTYD

SEQ ID NO: 362 - protein, 8 aa, synthetic construct
ISTSGTWT

SEQ ID NO: 363 - protein, 14 aa, synthetic construct
ASLIYGDYEAGLDY

SEQ ID NO: 364 - protein, 123 aa, synthetic construct
DVQLVESGGGLVQPGGSLRLSCAASGFSFGDYDMGWFRQAPGKEREFVAGIGSGGSTYYVDSLKGRFTISRDNSKNTVYLQMNSLRPEDTAVYYCARVAVSGTTRDYFDYWGQGSLVTVSSPP

SEQ ID NO: 365 - protein, 8 aa, synthetic construct
GFSFGDYD

SEQ ID NO: 366 - protein, 7 aa, synthetic construct
IGSGGST

SEQ ID NO: 367 - protein, 15 aa, synthetic construct
ARVAVSGTTRDYFDY

SEQ ID NO: 368 - protein, 122 aa, synthetic construct
DVQLVESGGGLVQPGGSLRLSCAASGFVFSDYDMGWFRQAPGKEREFVAGITGSTGGTYYVDSLKGRFTISRDNSKNTVYLQMNSLRPEDTAVYYCARVGAGGPEWFDYWGQGTLVTVSSPP

SEQ ID NO: 369 - protein, 8 aa, synthetic construct
GFVFSDYD

SEQ ID NO: 370 - protein, 8 aa, synthetic construct
ITGSTGGT

SEQ ID NO: 371 - protein, 13 aa, synthetic construct
ARVGAGGPEWFDY

SEQ ID NO: 372 - protein, 124 aa, synthetic construct
DVQLVESGGGLVQPGGSLRLSCAASGFDFSNHAMGWFRQAPGKEREFVAGVSGGAGGTYYVDSLKGRFTISRDNSKNTVYLQMNSLRPEDTAVYYCARECSGGSCSNYFDYWGQGTLVTVSSPP

SEQ ID NO: 373 - protein, 8 aa, synthetic construct
GFDFSNHA

SEQ ID NO: 374 - protein, 8 aa, synthetic construct
VSGGAGGT

SEQ ID NO: 375 - protein, 15 aa, synthetic construct
ARECSGGSCSNYFDY

SEQ ID NO: 376 - protein, 122 aa, synthetic construct
EVQLVESGGGLVQPGGSLRLSCAASGFTSSDHYMAWYRQAPGKGRELVAGISGPGVSTSYADSVKGRFTISRDNAKNTLYLQMNSLRPEDTAVYYCARFRPVGAIPFDLWGRGTLVTVSSPP

SEQ ID NO: 377 - protein, 8 aa, synthetic construct
GFTSSDHY

SEQ ID NO: 378 - protein, 8 aa, synthetic construct
ISGPGVST

SEQ ID NO: 379 - protein, 13 aa, synthetic construct
ARFRPVGAIPFDL

SEQ ID NO: 380 - protein, 127 aa, synthetic construct
EVQLVESGGGLVQPGGSLRLSCAASGFNFQDFAMGWFRQAPGKGRELVAAMSGSGSVTYYPDSVEGRFTISRDNAKRMVYLQMNSLRAEDTAVYYCARGIAAAGPTVDENWFDPWGQGTLVTVSSPP

SEQ ID NO: 381 - protein, 8 aa, synthetic construct
GFNFQDFA

SEQ ID NO: 382 - protein, 8 aa, synthetic construct
MSGSGSVT

SEQ ID NO: 383 - protein, 18 aa, synthetic construct
ARGIAAAGPTVDENWFDP

SEQ ID NO: 384 - protein, 120 aa, synthetic construct
EVQLVESGGGLVQPGGSLRLSCAASGFSFKTYDMGWFRQAPGKGRELVAAISVSGGWTYYPDSVEGRFTISRDNAKRMVYLQMNSLRAEDTAVYYCARETAAGEIDYWGQGTLVTVSSPP

SEQ ID NO: 385 - protein, 8 aa, synthetic construct
GFSFKTYD

SEQ ID NO: 386 - protein, 8 aa, synthetic construct
ISVSGGWT

SEQ ID NO: 387 - protein, 11 aa, synthetic construct
ARETAAGEIDY

SEQ ID NO: 388 - protein, 125 aa, synthetic construct
EVQLVESGGGLVQPGGSLRLSCAASGFSFNPYSMGWLRQAPGKEREFVAAISSAGGHTYYADSVKGRFTISRDNSKNTLYLQMNSLRPEDTAVYYCARDRNLYSSSLDAFDIWGQGTMVTVSSPP

SEQ ID NO: 389 - protein, 8 aa, synthetic construct
GFSFNPYS

SEQ ID NO: 390 - protein, 8 aa, synthetic construct
ISSAGGHT

SEQ ID NO: 391 - protein, 16 aa, synthetic construct
ARDRNLYSSSLDAFDI

SEQ ID NO: 392 - protein, 121 aa, synthetic construct
EVQLVESGGGLVQPGGSLRLSCAASGFTFATHSMGWFRQAPGKGRELVAAISGPGDFTYYPDSVEGRFTISRDNAKRMVYLQMNSLRAEDTAVYYCARDEEFDYELDYWGQGTLVTVSSPP

SEQ ID NO: 393 - protein, 8 aa, synthetic construct
GFTFATHS

SEQ ID NO: 394 - protein, 8 aa, synthetic construct
ISGPGDFT

SEQ ID NO: 395 - protein, 12 aa, synthetic construct
ARDEEFDYELDY

SEQ ID NO: 396 - protein, 118 aa, synthetic construct
EVQLVESGGGLVQPGGSLRLSCAASGFTLPTYGMGWLRQAPGKEREFVAAIGGSGDWTYYADSVKGRFTISRDNSKNTLYLQMNSLRPEDTAVYYCARDIGSYAYWGQGIRVTVSSPP

SEQ ID NO: 397 - protein, 8 aa, synthetic construct
GFTLPTYG

SEQ ID NO: 398 - protein, 8 aa, synthetic construct
IGGSGDWT

SEQ ID NO: 399 - protein, 9 aa, synthetic construct
ARDIGSYAY

SEQ ID NO: 400 - protein, 121 aa, synthetic construct
EVQLVESGGGLVQPGGSLRLSCAASGFAFSTSAMGWFRQAPGKGRELVAAITGSGGMTYYPDSVEGRFTISRDNAKRMVYLQMNSLRAEDTAVYYCARDEEFTSSFDYWGQGTLVTVSSPP

SEQ ID NO: 401 - protein, 8 aa, synthetic construct
GFAFSTSA

SEQ ID NO: 402 - protein, 8 aa, synthetic construct
ITGSGGMT

SEQ ID NO: 403 - protein, 12 aa, synthetic construct
ARDEEFTSSFDY

SEQ ID NO: 404 - protein, 123 aa, synthetic construct
EVQLVESGGGLVQPGGSLRLSCAASGFSLDEYGMAWYRQAPGKGRELVAGIGASGHSTSYADSVKGRFTISRDNAKNTLYLQMNSLRPEDTAVYYCARHWLGWSSGYPDYWGQGTLVTVSSPP

SEQ ID NO: 405 - protein, 8 aa, synthetic construct
GFSLDEYG

SEQ ID NO: 406 - protein, 8 aa, synthetic construct
IGASGHST

SEQ ID NO: 407 - protein, 14 aa, synthetic construct
ARHWLGWSSGYPDY

SEQ ID NO: 408 - protein, 119 aa, synthetic construct
EVQLVESGGGLVQPGGSLRLSCAASGFIFSGADMGWFRQAPGKGRELVAAISESGDWTYYPDSVEGRFTISRDNAKRMVYLQMNSLRAEDTAVYYCARFPDAAPGVWGKGTTVTVSSPP

SEQ ID NO: 409 - protein, 8 aa, synthetic construct
GFIFSGAD

SEQ ID NO: 410 - protein, 8 aa, synthetic construct
ISESGDWT

SEQ ID NO: 411 - protein, 10 aa, synthetic construct
ARFPDAAPGV

SEQ ID NO: 412 - protein, 117 aa, synthetic construct
EVQLVESGGGLVQPGGSLRLSCAASGFTFSNVDMGWLRQAPGKEREFVAAISGGHGDTYYADSVKGRFTISRDNSKNTLYLQMNSLRPEDTAVYYCAILETGDYWGQGTLVTVSSPP

SEQ ID NO: 413 - protein, 8 aa, synthetic construct
GFTFSNVD

SEQ ID NO: 414 - protein, 8 aa, synthetic construct
ISGGHGDT

SEQ ID NO: 415 - protein, 8 aa, synthetic construct
AILETGDY

SEQ ID NO: 416 - protein, 122 aa, synthetic construct
DVQLVESGGGLVQPGGSLRLSCAASGFLFGDYDMGWFRQAPGKEREFVAGVSHSGSSTYYVDSLKGRFTISRDNSKNTVYLQMNSLRPEDTAVYYCARTAVAGPAYFDYWGQGTLVTVSSPP

SEQ ID NO: 417 - protein, 8 aa, synthetic construct
GFLFGDYD

SEQ ID NO: 418 - protein, 8 aa, synthetic construct
VSHSGSST

SEQ ID NO: 419 - protein, 13 aa, synthetic construct
ARTAVAGPAYFDY

SEQ ID NO: 420 - protein, 122 aa, synthetic construct
DVQLVESGGGLVQPGGSLRLSCAASGFNFEEYGMGWFRQAPGKEREFVAGISGSGSGTYYVDSLKGRFTISRDNSKNTVYLQMNSLRPEDTAVYYCARVGVTTVTYFDYWGQGTLVTVSSPP

SEQ ID NO: 421 - protein, 8 aa, synthetic construct
GFNFEEYG

SEQ ID NO: 422 - protein, 8 aa, synthetic construct
ISGSGSGT

SEQ ID NO: 423 - protein, 13 aa, synthetic construct
ARVGVTTVTYFDY

SEQ ID NO: 424 - protein, 122 aa, synthetic construct
EVQLVESGGGLVQPGGSLRLSCAASGFTVSNHFMGWLRQAPGKEREFVAAITGSTGSTYYADSVKGRFTISRDNSKNTLYLQMNSLRPEDTAVYYCARDTFSSGYPPDYWGQGTLVTVSSPP

SEQ ID NO: 425 - protein, 8 aa, synthetic construct
GFTVSNHF

SEQ ID NO: 426 - protein, 8 aa, synthetic construct
ITGSTGST

SEQ ID NO: 427 - protein, 13 aa, synthetic construct
ARDTFSSGYPPDY

SEQ ID NO: 428 - protein, 122 aa, synthetic construct
DVQLVESGGGLVQPGGSLRLSCAASGFKFSTYDMGWFRQAPGKEREFVAGISVYAGSTYYVDSLKGRFTISRDNSKNTVYLQMNSLRPEDTAVYYCARTAVAGPGYFDYWGQGTLVTVSSPP

SEQ ID NO: 429 - protein, 8 aa, synthetic construct
GFKFSTYD

SEQ ID NO: 430 - protein, 8 aa, synthetic construct
ISVYAGST

SEQ ID NO: 431 - protein, 13 aa, synthetic construct
ARTAVAGPGYFDY

SEQ ID NO: 432 - protein, 124 aa, synthetic construct
DVQLVESGGGLVQPGGSLRLSCAASGFPSSSYSMGWFRQAPGKEREFVAGVSGSGTSTYYVDSLKGRFTISRDNSKNTVYLQMNSLRPEDTAVYYCAREPLSGSTPDYFDYWGQGTLVTVSSPP

SEQ ID NO: 433 - protein, 8 aa, synthetic construct
GFPSSSYS

SEQ ID NO: 434 - protein, 8 aa, synthetic construct
VSGSGTST

SEQ ID NO: 435 - protein, 15 aa, synthetic construct
AREPLSGSTPDYFDY

SEQ ID NO: 436 - protein, 121 aa, synthetic construct
EVQLVESGGGLVQPGGSLRLSCAASGFSFHDHAMGWFRQAPGKGRELVAAISTSGGFTYYPDSVEGRFTISRDNAKRMVYLQMNSLRAEDTAVYYCARDTLEPQAFDIWGQGTMVTVSSPP

SEQ ID NO: 437 - protein, 8 aa, synthetic construct
GFSFHDHA

SEQ ID NO: 438 - protein, 8 aa, synthetic construct
ISTSGGFT

SEQ ID NO: 439 - protein, 12 aa, synthetic construct
ARDTLEPQAFDI

SEQ ID NO: 440 - protein, 124 aa, synthetic construct
DVQLVESGGGLVQPGGSLRLSCAASGFAVSSNEMGWFRQAPGKEREFVAGIIGTGGSTYYVDSLKGRFTISRDNSKNTVYLQMNSLRPEDTAVYYCARVAAAGTGGDYFDYWGQGTLVTVSSPP

SEQ ID NO: 441 - protein, 8 aa, synthetic construct
GFAVSSNE

SEQ ID NO: 442 - protein, 8 aa, synthetic construct
IIGTGGST

SEQ ID NO: 443 - protein, 15 aa, synthetic construct
ARVAAAGTGGDYFDY

SEQ ID NO: 444 - protein, 118 aa, synthetic construct
EVQLVESGGGLVQPGGSLRLSCAASGLPFSSYSMGWLRQAPGKEREFVAAIGGSGDWTYYADSVKGRFTISRDNSKNTLYLQMNSLRPEDTAVYYCTAITTTMDVWGQGTTVTVSSPP

SEQ ID NO: 445 - protein, 8 aa, synthetic construct
GLPFSSYS

SEQ ID NO: 446 - skipped

SEQ ID NO: 447 - protein, 9 aa, synthetic construct
TAITTTMDV

SEQ ID NO: 448 - protein, 123 aa, synthetic construct
DVQLVESGGGLVQPGGSLRLSCAASGFDFSSHEMGWFRQAPGKEREFVAGITGSTGSTYYVDSLKGRFTISRDNSKNTVYLQMNSLRPEDTAVYYCARVPSAAGRNYFDYWGQGTLVTVSSPP

SEQ ID NO: 449 - protein, 8 aa, synthetic construct
GFDFSSHE

SEQ ID NO: 450 - skipped

SEQ ID NO: 451 - protein, 14 aa, synthetic construct
ARVPSAAGRNYFDY

SEQ ID NO: 452 - protein, 122 aa, synthetic construct
DVQLVESGGGLVQPGGSLRLSCAASGFTLSSAGMGWFRQAPGKEREFVAGISSDEGGTYYVDSLKGRFTISRDNSKNTVYLQMNSLRPEDTAVYYCARVSIAGSSYFDYWGQGTLVTVSSPP

SEQ ID NO: 453 - protein, 8 aa, synthetic construct
GFTLSSAG

SEQ ID NO: 454 - protein, 8 aa, synthetic construct
ISSDEGGT

SEQ ID NO: 455 - protein, 13 aa, synthetic construct
ARVSIAGSSYFDY

SEQ ID NO: 456 - protein, 122 aa, synthetic construct
DVQLVESGGGLVQPGGSLRLSCAASGFTLTDYEMGWFRQAPGKEREFVAGISGIGGTTYYVDSLKGRFTISRDNSKNTVYLQMNSLRPEDTAVYYCARVAATGAEYFQHWGQGTLVTVSSPP

SEQ ID NO: 457 - protein, 8 aa, synthetic construct
GFTLTDYE

SEQ ID NO: 458 - protein, 8 aa, synthetic construct
ISGIGGTT

SEQ ID NO: 459 - protein, 13 aa, synthetic construct
ARVAATGAEYFQH

SEQ ID NO: 460 - protein, 122 aa, synthetic construct
DVQLVESGGGLVQPGGSLRLSCAASGFSFSSYDMGWFRQAPGKEREFVAGVSPSGGDTYYVDSLKGRFTISRDNSKNTVYLQMNSLRPEDTAVYYCARVGLVGATYFDYWGQGTLVTVSSPP

SEQ ID NO: 461 - protein, 8 aa, synthetic construct
GFSFSSYD

SEQ ID NO: 462 - protein, 8 aa, synthetic construct
VSPSGGDT

SEQ ID NO: 463 - protein, 13 aa, synthetic construct
ARVGLVGATYFDY

SEQ ID NO: 464 - protein, 123 aa, synthetic construct
DVQLVESGGGLVQPGGSLRLSCAASGFSFSTDAMGWFRQAPGKEREFVAGIDASGGSTYYVDSLKGRFTISRDNSKNTVYLQMNSLRPEDTAVYYCAREAAVAGPYFFDYWGQGALVTVSSPP

SEQ ID NO: 465 - protein, 8 aa, synthetic construct
GFSFSTDA

SEQ ID NO: 466 - protein, 8 aa, synthetic construct
IDASGGST

SEQ ID NO: 467 - protein, 14 aa, synthetic construct
AREAAVAGPYFFDY

SEQ ID NO: 468 - protein, 122 aa, synthetic construct
DVQLVESGGGLVQPGGSLRLSCAASGFTLTDYDMGWFRQAPGKEREFVAGISGSTPSTYYVDSLKGRFTISRDNSKNTVYLQMNSLRPEDTAVYYCARVGKTGATYFDYWGQGTLVTVSSPP

SEQ ID NO: 469 - protein, 8 aa, synthetic construct
GFTLTDYD

SEQ ID NO: 470 - protein, 8 aa, synthetic construct
ISGSTPST

SEQ ID NO: 471 - protein, 13 aa, synthetic construct
ARVGKTGATYFDY

SEQ ID NO: 472 - protein, 114 aa, synthetic construct
EVQLVESGGGLVQPGGSLRLSCAASGFTSTNYVMGWFRQAPGKGRELVAAISAGGTDTYYPDSVEGRFTISRDNAKRMVYLQMNSLRAEDTAVYYCANHQAVGQGTLVTVSSPP

SEQ ID NO: 473 - protein, 8 aa, synthetic construct
GFTSTNYV

SEQ ID NO: 474 - protein, 8 aa, synthetic construct
ISAGGTDT

SEQ ID NO: 475 - protein, 5 aa, synthetic construct
ANHQA

SEQ ID NO: 476 - protein, 119 aa, synthetic construct
EVQLVESGGGLVQPGGSLRLSCAASGFDFSNADMGWLRQAPGKEREFVAAVSSTGGTTYYADSVKGRFTISRDNSKNTLYLQMNSLRPEDTAVYYCARADVVTSDYWGQGTLVTVSSPP

SEQ ID NO: 477 - protein, 8 aa, synthetic construct
GFDFSNAD

SEQ ID NO: 478 - protein, 8 aa, synthetic construct
VSSTGGTT

SEQ ID NO: 479 - protein, 10 aa, synthetic construct
ARADVVTSDY

SEQ ID NO: 480 - protein, 122 aa, synthetic construct
DVQLVESGGGLVQPGGSLRLSCAASGFTFTAYDMGWFRQAPGKEREFVAGINPEGSETYYVDSLKGRFTISRDNSKNTVYLQMNSLRPEDTAVYYCARVGYSSSSFFDYWGQGTLVTVSSPP

SEQ ID NO: 481 - protein, 8 aa, synthetic construct
GFTFTAYD

SEQ ID NO: 482 - protein, 8 aa, synthetic construct
INPEGSET

SEQ ID NO: 483 - protein, 13 aa, synthetic construct
ARVGYSSSSFFDY

SEQ ID NO: 484 - protein, 122 aa, synthetic construct
DVQLVESGGGLVQPGGSLRLSCAASGFSFTTSGMGWFRQAPGKEREFVAGISGSGSSTYYVDSLKGRFTISRDNSKNTVYLQMNSLRPEDTAVYYCARVAQAGGEYFDYWGQGTLVTVSSPP

SEQ ID NO: 485 - protein, 8 aa, synthetic construct
GFSFTTSG

SEQ ID NO: 486 - protein, 8 aa, synthetic construct
ISGSGSST

SEQ ID NO: 487 - protein, 13 aa, synthetic construct
ARVAQAGGEYFDY

SEQ ID NO: 488 - protein, 122 aa, synthetic construct
DVQLVESGGGLVQPGGSLRLSCAASGFPFSKFDMGWFRQAPGKEREFVAGLGGSGGSTYYVDSLKGRFTISRDNSKNTVYLQMNSLRPEDTAVYYCARVGSVGATFFDYWGQGTLVTVSSPP

SEQ ID NO: 489 - protein, 8 aa, synthetic construct
GFPFSKFD

SEQ ID NO: 490 - protein, 8 aa, synthetic construct
LGGSGGST

SEQ ID NO: 491 - protein, 13 aa, synthetic construct
ARVGSVGATFFDY

SEQ ID NO: 492-500 - skipped (9 entries)

SEQ ID NO: 501 - protein, 122 aa, synthetic construct
EVQLVESGGGLVQPGGSLRLSCAASGFDFSTTWMGWLRQAPGKEREFVAAINAPGSETYYADSVKGRFTISRDNSKNTLYLQMNSLRPEDTAVYYCARDEAVAGYHWWFDPWGQGTLVTVSS

SEQ ID NO: 502 - protein, 8 aa, synthetic construct
GFDFSTTW

SEQ ID NO: 503 - protein, 8 aa, synthetic construct
INAPGSET

SEQ ID NO: 504 - protein, 15 aa, synthetic construct
ARDEAVAGYHWWFDP

SEQ ID NO: 505 - protein, 122 aa, synthetic construct
EVQLVESGGGLVQPGGSLRLSCAASGFTVSDYDMAWYRQAPGKGRELVAGINDEGTTTSYADSVKGRFTISRDNAKNTLYLQMNSLRPEDTAVYYCARVVSGQQLVFPLDYWGQGSLVTVSS

SEQ ID NO: 506 - protein, 8 aa, synthetic construct
GFTVSDYD

SEQ ID NO: 507 - protein, 8 aa, synthetic construct
INDEGTTT

SEQ ID NO: 508 - protein, 15 aa, synthetic construct
ARVVSGQQLVFPLDY

SEQ ID NO: 509 - protein, 121 aa, synthetic construct
DVQLVESGGGLVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSS

SEQ ID NO: 510 - protein, 8 aa, synthetic construct
GLPFSDYS

SEQ ID NO: 511 - protein, 8 aa, synthetic construct
ISGSSITT

SEQ ID NO: 512 - protein, 14 aa, synthetic construct
ATSGYSYSAGGMDV

SEQ ID NO: 513 - protein, 121 aa, synthetic construct
EVQLVESGGGLVQPGGSLRLSCAASGFVFSDHAMGWFRQAPGKGRELVAAISAPVGVTYYPDSVEGRFTISRDNAKRMVYLQMNSLRAEDTAVYYCAREDSSGYLDWFDPWGQGTLVTVSS

SEQ ID NO: 514 - protein, 8 aa, synthetic construct
GFVFSDHA

SEQ ID NO: 515 - protein, 8 aa, synthetic construct
ISAPVGVT

SEQ ID NO: 516 - protein, 14 aa, synthetic construct
AREDSSGYLDWFDP

SEQ ID NO: 517 - protein, 123 aa, synthetic construct
DVQLVESGGGLVQPGGSLRLSCAASGLSFSDYSMGWFRQAPGKEREFVAGISGSSITTYYADSVKGRFTISRDNSKNTVYLQMNSLRPEDTAVYYCASLGPTYYYDSSGDDYWGQGTLVTVSS

SEQ ID NO: 518 - protein, 8 aa, synthetic construct
GLSFSDYS

SEQ ID NO: 519 - skipped

SEQ ID NO: 520 - protein, 16 aa, synthetic construct
ASLGPTYYYDSSGDDY

SEQ ID NO: 521 - protein, 123 aa, synthetic construct
EVQLVESGGGLVQPGGSLRLSCAASGFTVNDYDMAWYRQAPGKGRELVAGIDASGTKTSYADSVKGRFTISRDNAKNTLYLQMNSLRPEDTAVYYCARHVWGDHGGWYPLDYWGQGTLVTVSS

SEQ ID NO: 522 - protein, 8 aa, synthetic construct
GFTVNDYD

SEQ ID NO: 523 - protein, 8 aa, synthetic construct
IDASGTKT

SEQ ID NO: 524 - protein, 16 aa, synthetic construct
ARHVWGDHGGWYPLDY

SEQ ID NO: 525 - protein, 124 aa, synthetic construct
EVQLVESGGGLVQPGGSLRLSCAASGFSVNDYDMAWYRQAPGKGRELVAGLTESGGSTSYADSVKGRFTISRDNAKNTLYLQMNSLRPEDTAVYYCARVVVSDSSGYYYPLDYWGQGTLVTVSS

SEQ ID NO: 526 - protein, 8 aa, synthetic construct
GFSVNDYD

SEQ ID NO: 527 - protein, 8 aa, synthetic construct
LTESGGST

SEQ ID NO: 528 - protein, 17 aa, synthetic construct
ARVVVSDSSGYYYPLDY

SEQ ID NO: 529 - protein, 117 aa, synthetic construct
EVQLVESGGGLVQPGGSLRLSCAASGISFSDYGMGWFRQAPGKGRELVAAISSKGGATYYPDSVEGRFTISRDNAKRMVYLQMNSLRAEDTAVYYCVGGEQWLEDNWGQGTLVTVSS

SEQ ID NO: 530 - protein, 8 aa, synthetic construct
GISFSDYG

SEQ ID NO: 531 - protein, 8 aa, synthetic construct
ISSKGGAT

SEQ ID NO: 532 - protein, 10 aa, synthetic construct
VGGEQWLEDN

SEQ ID NO: 533 - protein, 124 aa, synthetic construct
EVQLVESGGGLVQPGGSLRLSCAASGFTFSDYTMGWFRQAPGKGRELVAAISGPGGITYYPDSVEGRFTISRDNAKRMVYLQMNSLRAEDTAVYYCAKEEPAGYSSGWYGFDYWGQGTLVTVSS

SEQ ID NO: 534 - protein, 8 aa, synthetic construct
GFTFSDYT

SEQ ID NO: 535 - protein, 8 aa, synthetic construct
ISGPGGIT

SEQ ID NO: 536 - protein, 17 aa, synthetic construct
AKEEPAGYSSGWYGFDY

SEQ ID NO: 537 - protein, 122 aa, synthetic construct
EVQLVESGGGLVQPGGSLRLSCAASGFTFGDTWMGWLRQAPGKEREFVAAIGSSGSDTYYADSVKGRFTISRDNSKNTLYLQMNSLRPEDTAVYYCARDELISGYAWYFDNWGQGTLVTVSS

SEQ ID NO: 538 - protein, 8 aa, synthetic construct
GFTFGDTW

SEQ ID NO: 539 - protein, 8 aa, synthetic construct
IGSSGSDT

SEQ ID NO: 540 - protein, 15 aa, synthetic construct
ARDELISGYAWYFDN

SEQ ID NO: 541 - protein, 116 aa, synthetic construct
EVQLVESGGGLVQPGGSLRLSCAASGLDFHNYAMGWFRQAPGKGRELVAAISAPGGATYYPDSVEGRFTISRDNAKRMVYLQMNSLRAEDTAVYYCAALGDYDDYWGQGTLVTVSS

SEQ ID NO: 542 - protein, 8 aa, synthetic construct
GLDFHNYA

SEQ ID NO: 543 - protein, 8 aa, synthetic construct
ISAPGGAT

SEQ ID NO: 544 - protein, 9 aa, synthetic construct
AALGDYDDY

SEQ ID NO: 545 - protein, 120 aa, synthetic construct
DVQLVESGGGLVQPGGSLRLSCAASGFTFSDYDMGWFRQAPGKEREFVAAIMGSGGTTYYADSVKGRFTISRDNSKNTVYLQMNSLRPEDTAVYYCASQYSYGLSYFDYWGQGTLVTVSS

SEQ ID NO: 546 - protein, 8 aa, synthetic construct
GFTFSDYD

SEQ ID NO: 547 - protein, 8 aa, synthetic construct
IMGSGGTT

SEQ ID NO: 548 - protein, 13 aa, synthetic construct
ASQYSYGLSYFDY

SEQ ID NO: 549 - protein, 121 aa, synthetic construct
DVQLVESGGGLVQPGGSLRLSCAASGIPFSDYSMGWFRQAPGKEREFVAGISGSSITTYYADSVKGRFTISRDNSKNTVYLQMNSLRPEDTAVYYCASLGYSYGYGGFDYWGQGTLVTVSS

SEQ ID NO: 550 - protein, 8 aa, synthetic construct
GIPFSDYS

SEQ ID NO: 551 - skipped

SEQ ID NO: 552 - protein, 14 aa, synthetic construct
ASLGYSYGYGGFDY

SEQ ID NO: 553 - protein, 117 aa, synthetic construct
EVQLVESGGGLVQPGGSLRLSCAASGFKFEEYAMGWFRQAPGKGRELVAAISNKGGATYYPDSVEGRFTISRDNAKRMVYLQMNSLRAEDTAVYYCAGGSGWLTDYWGRGTLVTVSS

SEQ ID NO: 554 - protein, 8 aa, synthetic construct
GFKFEEYA

SEQ ID NO: 555 - protein, 8 aa, synthetic construct
ISNKGGAT

SEQ ID NO: 556 - protein, 10 aa, synthetic construct
AGGSGWLTDY

SEQ ID NO: 557 - protein, 117 aa, synthetic construct
EVQLVESGGGLVQPGGSLRLSCAASGFAFGDYAMGWFRQAPGKGRELVAAISAPGGATYYPDSVEGRFTISRDNAKRMVYLQMNSLRAEDTAVYYCTTGEQWLDDYWGQGTLVTVSS

SEQ ID NO: 558 - protein, 8 aa, synthetic construct
GFAFGDYA

SEQ ID NO: 559 - skipped

SEQ ID NO: 560 - protein, 10 aa, synthetic construct
TTGEQWLDDY

SEQ ID NO: 561 - protein, 117 aa, synthetic construct
EVQLVESGGGLVQPGGSLRLSCAASGFDFDNYGMGWFRQAPGKGRELVAAISGYGGATYYPDSVEGRFTISRDNAKRMVYLQMNSLRAEDTAVYYCAGGSGWLSDSWGQGTMVTVSS

SEQ ID NO: 562 - protein, 8 aa, synthetic construct
GFDFDNYG

SEQ ID NO: 563 - protein, 8 aa, synthetic construct
ISGYGGAT

SEQ ID NO: 564 - protein, 10 aa, synthetic construct
AGGSGWLSDS

SEQ ID NO: 565 - protein, 117 aa, synthetic construct
EVQLVESGGGLVQPGGSLRLSCAASGGTFSDYAMGWFRQAPGKGRELVAAISGPGGATYYPDSVEGRFTISRDNAKRMVYLQMNSLRAEDTAVYYCAGGQGWLTDYWGQGTLVTVSS

SEQ ID NO: 566 - protein, 8 aa, synthetic construct
GGTFSDYA

SEQ ID NO: 567 - protein, 8 aa, synthetic construct
ISGPGGAT

SEQ ID NO: 568 - protein, 10 aa, synthetic construct
AGGQGWLTDY

SEQ ID NO: 569 - protein, 122 aa, synthetic construct
DVQLVESGGGLVQPGGSLRLSCAASGLSFSEYSMGWFRQAPGKEREFVAGISGSSITTYYADSVKGRFTISRDNSKNTVYLQMNSLRPEDTAVYYCASLGLTYGDSDQFDYWGQGTLVTVSS

SEQ ID NO: 570 - protein, 8 aa, synthetic construct
GLSFSEYS

SEQ ID NO: 571 - skipped

SEQ ID NO: 572 - protein, 15 aa, synthetic construct
ASLGLTYGDSDQFDY

SEQ ID NO: 573 - protein, 117 aa, synthetic construct
EVQLVESGGGLVQPGGSLRLSCAASGLTISDYAMGWFRQAPGKGRELVAAISGPGGATYYPDSVEGRFTISRDNAKRMVYLQMNSLRAEDTAVYYCTTGFYSLEDYWGQGTLVTVSS

SEQ ID NO: 574 - protein, 8 aa, synthetic construct
GLTISDYA

SEQ ID NO: 575 - skipped

SEQ ID NO: 576 - protein, 10 aa, synthetic construct
TTGFYSLEDY

SEQ ID NO: 577 - protein, 116 aa, synthetic construct
EVQLVESGGGLVQPGGSLRLSCAASGFPFSDYAMGWFRQAPGKGRELVAAISGHGGATYYPDSVEGRFTISRDNAKRMVYLQMNSLRAEDTAVYYCAGLGDYEDYWGQGTLVTVSS

SEQ ID NO: 578 - protein, 8 aa, synthetic construct
GFPFSDYA

SEQ ID NO: 579 - protein, 8 aa, synthetic construct
ISGHGGAT

SEQ ID NO: 580 - protein, 9 aa, synthetic construct
AGLGDYEDY

SEQ ID NO: 581 - protein, 116 aa, synthetic construct
EVQLVESGGGLVQPGGSLRLSCAASGFDFSNYGMGWFRQAPGKGRELVAAISNKGGATYYPDSVEGRFTISRDNAKRMVYLQMNSLRAEDTAVYYCAGGSYLEDYWGQGTLVTVSS

SEQ ID NO: 582 - protein, 8 aa, synthetic construct
GFDFSNYG

SEQ ID NO: 583 - skipped

SEQ ID NO: 584 - protein, 9 aa, synthetic construct
AGGSYLEDY

SEQ ID NO: 585 - protein, 117 aa, synthetic construct
EVQLVESGGGLVQPGGSLRLSCAASGLNFDNYGMGWFRQAPGKGRELVAAISAGSGATYYPDSVEGRFTISRDNAKRMVYLQMNSLRAEDTAVYYCAGGSGWLTDSWGQGSLVTVSS

SEQ ID NO: 586 - protein, 8 aa, synthetic construct
GLNFDNYG

SEQ ID NO: 587 - protein, 8 aa, synthetic construct
ISAGSGAT

SEQ ID NO: 588 - protein, 10 aa, synthetic construct
AGGSGWLTDS

SEQ ID NO: 589 - protein, 121 aa, synthetic construct
EVQLVESGGGLVQPGGSLRLSCAASGFSFSDAAMGWFRQAPGKGRELVAAISAPGGVTYYPDSVEGRFTISRDNAKRMVYLQMNSLRAEDTAVYYCAREDSSGWINWFDPWGQGTLVTVSS

SEQ ID NO: 590 - protein, 8 aa, synthetic construct
GFSFSDAA

SEQ ID NO: 591 - protein, 8 aa, synthetic construct
ISAPGGVT

SEQ ID NO: 592 - protein, 14 aa, synthetic construct
AREDSSGWINWFDP

SEQ ID NO: 593 - protein, 116 aa, synthetic construct
EVQLVESGGGLVQPGGSLRLSCAASGFDFNDHAMGWFRQAPGKGRELVAAISNKGGATYYPDSVEGRFTISRDNAKRMVYLQMNSLRAEDTAVYYCARLEDTATTWGQGTLVTVSS

SEQ ID NO: 594 - protein, 8 aa, synthetic construct
GFDFNDHA

SEQ ID NO: 595 - skipped

SEQ ID NO: 596 - protein, 9 aa, synthetic construct
ARLEDTATT

SEQ ID NO: 597 - protein, 117 aa, synthetic construct
EVQLVESGGGLVQPGGSLRLSCAASGFPFSDYAMGWFRQAPGKGRELVAAISGLGGATYYPDSVEGRFTISRDNAKRMVYLQMNSLRAEDTAVYYCAGGEDYIIDHWGQGVLVTVSS

SEQ ID NO: 598 - skipped

SEQ ID NO: 599 - protein, 8 aa, synthetic construct
ISGLGGAT

SEQ ID NO: 600 - protein, 10 aa, synthetic construct
AGGEDYIIDH

SEQ ID NO: 601 - protein, 121 aa, synthetic construct
DVQLVESGGGLVQPGGSLRLSCAASGITFSDYSMGWFRQAPGKEREFVAGISGSSITTYYADSVKGRFTISRDNSKNTVYLQMNSLRPEDTAVYYCATAGLSGSYGGLDYWGQGTLVTVSS

SEQ ID NO: 602 - protein, 8 aa, synthetic construct
GITFSDYS

SEQ ID NO: 603 - skipped

SEQ ID NO: 604 - protein, 14 aa, synthetic construct
ATAGLSGSYGGLDY

SEQ ID NO: 605 - protein, 123 aa, synthetic construct
EVQLVESGGGLVQPGGSLRLSCAASGFDFNDFAMGWFRQAPGKGRELVAAISGPGGATYYPDSVEGRFTISRDNAKRMVYLQMNSLRAEDTAVYYCAKLPVTTEGPGDAFDIWGQGTMVTVSS

SEQ ID NO: 606 - protein, 8 aa, synthetic construct
GFDFNDFA

SEQ ID NO: 607 - skipped

SEQ ID NO: 608 - protein, 16 aa, synthetic construct
AKLPVTTEGPGDAFDI

SEQ ID NO: 609 - protein, 118 aa, synthetic construct
DVQLVESGGGLVQPGGSLRLSCAASGFDFSDAAMGWFRQAPGKGRELVAAISAPGGVTYYPDSVEGRFTISRDNAKRMVYLQMNSLRPEDTAVYYCTTDFVSGYLDYWGQGILVTVSS

SEQ ID NO: 610 - protein, 8 aa, synthetic construct
GFDFSDAA

SEQ ID NO: 611 - skipped

SEQ ID NO: 612 - protein, 11 aa, synthetic construct
TTDFVSGYLDY

SEQ ID NO: 613 - protein, 116 aa, synthetic construct
EVQLVESGGGLVQPGGSLRLSCAASGVDFSNYAMGWFRQAPGKGRELVAAISSYGGATYYPDSVEGRFTISRDNAKRMVYLQMNSLRAEDTAVYYCAVLGDYEDYWGQGTLVTVSS

SEQ ID NO: 614 - protein, 8 aa, synthetic construct
GVDFSNYA

SEQ ID NO: 615 - protein, 8 aa, synthetic construct
ISSYGGAT

SEQ ID NO: 616 - protein, 9 aa, synthetic construct
AVLGDYEDY

SEQ ID NO: 617 - protein, 116 aa, synthetic construct
EVQLVESGGGLVQPGGSLRLSCAASGSAFGTYAMGWFRQAPGKGRELVAAISGPGGATYYPDSVEGRFTISRDNAKRMVYLQMNSLRAEDTAVYYCAALGDYFDYWGQGTLVTVSS

SEQ ID NO: 618 - protein, 8 aa, synthetic construct
GSAFGTYA

SEQ ID NO: 619 - skipped

SEQ ID NO: 620 - protein, 9 aa, synthetic construct
AALGDYFDY

SEQ ID NO: 621 - protein, 119 aa, synthetic construct
EVQLVESGGGLVQPGGSLRLSCAASGLTFSDYGMGWFRQAPGKGRELVAAISGPGGATYYPDSVEGRFTISRDNAKRMVYLQMNSLRAEDTAVYYCAALPDNDYGDYLWGQGTMVTVSS

SEQ ID NO: 622 - protein, 8 aa, synthetic construct
GLTFSDYG

SEQ ID NO: 623 - skipped

SEQ ID NO: 624 - protein, 12 aa, synthetic construct
AALPDNDYGDYL

SEQ ID NO: 625 - protein, 122 aa, synthetic construct
DVQLVESGGGLVQPGGSLRLSCAASGISFSDYSMGWFRQAPGKEREFVAGISGSSITTYYADSVKGRFTISRDNSKNTVYLQMNSLRPEDTAVYYCASLGLSQYSGYDEDYWGQGTLVTVSS

SEQ ID NO: 626 - protein, 8 aa, synthetic construct
GISFSDYS

SEQ ID NO: 627 - skipped

SEQ ID NO: 628 - protein, 15 aa, synthetic construct
ASLGLSQYSGYDEDY

SEQ ID NO: 629 - protein, 117 aa, synthetic construct
EVQLVESGGGLVQPGGSLRLSCAASGFNFDEFGMGWFRQAPGKGRELVAAISGYGGATYYPDSVEGRFTISRDNAKRMVYLQMNSLRAEDTAVYYCAGGSSSVEDYWGQGTLVTVSS

SEQ ID NO: 630 - protein, 8 aa, synthetic construct
GFNFDEFG

SEQ ID NO: 631 - skipped

SEQ ID NO: 632 - protein, 10 aa, synthetic construct
AGGSSSVEDY

SEQ ID NO: 633 - protein, 118 aa, synthetic construct
EVQLVESGGGLVQPGGSLRLSCAASGFDFEDYGMGWFRQAPGKGRELVAAISGLGGATYYPDSVEGRFTISRDNAKRMVYLQMNSLRAEDTAVYYCATLGEYGDYDYWGQGTLVTVSS

SEQ ID NO: 634 - protein, 8 aa, synthetic construct
GFDFEDYG

SEQ ID NO: 635 - skipped

SEQ ID NO: 636 - protein, 11 aa, synthetic construct
ATLGEYGDYDY

SEQ ID NO: 637 - protein, 116 aa, synthetic construct
EVQLVESGGGLVQPGGSLRLSCAASGLNFGDYAMGWFRQAPGKGRELVAAISNKGGATYYPDSVEGRFTISRDNAKRMVYLQMNSLRAEDTAVYYCATLEWELDYWGQGTLVTVSS

SEQ ID NO: 638 - protein, 8 aa, synthetic construct
GLNFGDYA

SEQ ID NO: 639 - skipped

SEQ ID NO: 640 - protein, 9 aa, synthetic construct
ATLEWELDY

SEQ ID NO: 641 - protein, 117 aa, synthetic construct
EVQLVESGGGLVQPGGSLRLSCAASGFKFGDYAMGWFRQAPGKGRELVAAISGPGGATYYPDSVEGRFTISRDNAKRMVYLQMNSLRAEDTAVYYCAGGAGWLTDYWGQGTLVTVSS

SEQ ID NO: 642 - protein, 8 aa, synthetic construct
GFKFGDYA

SEQ ID NO: 643 - skipped

SEQ ID NO: 644 - protein, 10 aa, synthetic construct
AGGAGWLTDY

SEQ ID NO: 645 - protein, 121 aa, synthetic construct
DVQLVESGGGLVQPGGSLRLSCAASGISLSDYSMGWFRQAPGKEREFVAGISGSSITTYYADSVKGRFTISRDNSKNTVYLQMNSLRPEDTAVYYCASLGFTMIVGGIDYWGQGTLVTVSS

SEQ ID NO: 646 - protein, 8 aa, synthetic construct
GISLSDYS

SEQ ID NO: 647 - skipped

SEQ ID NO: 648 - protein, 14 aa, synthetic construct
ASLGFTMIVGGIDY

SEQ ID NO: 649 - protein, 116 aa, synthetic construct
EVQLVESGGGLVQPGGSLRLSCAASGFDFSGFSMGWFRQAPGKGRELVAAISAPVGVTYYPDSVEGRFTISRDNAKRMVYLQMNSLRAEDTAVYYCVYLGDAFDIWGQGTMVTVSS

SEQ ID NO: 650 - protein, 8 aa, synthetic construct
GFDFSGFS

SEQ ID NO: 651 - skipped

SEQ ID NO: 652 - protein, 9 aa, synthetic construct
VYLGDAFDI

SEQ ID NO: 653 - protein, 118 aa, synthetic construct
DVQLVESGGGLVQPGGSLRLSCAASGFTISGNDMGWFRQAPGKEREFVAAIMGSGGTTYYADSVKGRFTISRDNSKNTVYLQMNSLRPEDTAVYYCAKQPPDWYFDLWGRGTLVTVSS

SEQ ID NO: 654 - protein, 8 aa, synthetic construct
GFTISGND

SEQ ID NO: 655 - skipped

SEQ ID NO: 656 - protein, 11 aa, synthetic construct
AKQPPDWYFDL

SEQ ID NO: 657 - protein, 122 aa, synthetic construct
EVQLVESGGGLVQPGGSLRLSCAASGFAFSDTWMGWLRQAPGKEREFVAAINAAGSDTYYADSVKGRFTISRDNSKNTLYLQMNSLRPEDTAVYYCARDEQISGYAWYFDLWGRGTLVTVSS

SEQ ID NO: 658 - protein, 8 aa, synthetic construct
GFAFSDTW

SEQ ID NO: 659 - protein, 8 aa, synthetic construct
INAAGSDT

SEQ ID NO: 660 - protein, 15 aa, synthetic construct
ARDEQISGYAWYFDL

SEQ ID NO: 661 - protein, 118 aa, synthetic construct
EVQLVESGGGLVQPGGSLRLSCAASGLALSSYDMAWYRQAPGKGRELVAGISAGGEHTSYADSVKGRFTISRDNAKNTLYLQMNSLRPEDTAVYYCARNFEIRYFNPWGQGTLVTVSS

SEQ ID NO: 662 - protein, 8 aa, synthetic construct
GLALSSYD

SEQ ID NO: 663 - protein, 8 aa, synthetic construct
ISAGGEHT

SEQ ID NO: 664 - protein, 11 aa, synthetic construct
ARNFEIRYFNP

SEQ ID NO: 665 - protein, 125 aa, synthetic construct
EVQLVESGGGLVQPGGSLRLSCAASGFNFEEYGMGWFRQAPGKGRELVAAISAPGGVTYYPDSVEGRFTISRDNAKRMVYLQMNSLKSEDTAVYYCARVADYGSGSYSSNWFDPWGQGTLVTVSS

SEQ ID NO: 666-667 - skipped (2 entries)

SEQ ID NO: 668 - protein, 18 aa, synthetic construct
ARVADYGSGSYSSNWFDP

SEQ ID NO: 669 - protein, 129 aa, synthetic construct
EVQLVESGGGLVQPGGSLRLSCAASGLVFSDYSMGWFRQAPGKGRELVAAISAPGGVTYYPDSVEGRFTISRDNAKRMVYLQMNSLRAEDTAVYYCARVCSEYCGGDWLSAGYWYFDLWGRGTLVTVSS

SEQ ID NO: 670 - protein, 8 aa, synthetic construct
GLVFSDYS

SEQ ID NO: 671 - skipped

SEQ ID NO: 672 - protein, 22 aa, synthetic construct
ARVCSEYCGGDWLSAGYWYFDL

SEQ ID NO: 673 - protein, 122 aa, synthetic construct
EVQLVESGGGLVQPGGSLRLSCAASGFSVSDYDMAWYRQAPGKGRELVAGISGSASHTSYADSVKGRFTISRDNAKNTLYLQMNSLRPEDTAVYYCTRVVSGQQLVFPLDYWGQGSLVTVSS

SEQ ID NO: 674 - protein, 8 aa, synthetic construct
GFSVSDYD

SEQ ID NO: 675 - protein, 8 aa, synthetic construct
ISGSASHT

SEQ ID NO: 676 - protein, 15 aa, synthetic construct
TRVVSGQQLVFPLDY

SEQ ID NO: 677 - protein, 120 aa, synthetic construct
EVQLVESGGGLVQPGGSLRLSCAASGFDFGGYGMGWFRQAPGKGRELVAAISSHGGATYYPDSVEGRFTISRDNAKRMVYLQMNSLRAEDTAVYYCASLGSDYGEGDDYWGQGTLVTVSS

SEQ ID NO: 678 - protein, 8 aa, synthetic construct
GFDFGGYG

SEQ ID NO: 679 - protein, 8 aa, synthetic construct
ISSHGGAT

SEQ ID NO: 680 - protein, 13 aa, synthetic construct
ASLGSDYGEGDDY

SEQ ID NO: 681 - protein, 117 aa, synthetic construct
EVQLVESGGGLVQPGGSLRLSCAASGITLEDYAMGWFRQAPGKGRELVAAISGLGGATYYPDSVEGRFTISRDNAKRMVYLQMNSLRAEDTAVYYCVGGSGWLSDYWGHGTLVTVSS

SEQ ID NO: 682 - protein, 8 aa, synthetic construct
GITLEDYA

SEQ ID NO: 683 - skipped

SEQ ID NO: 684 - protein, 10 aa, synthetic construct
VGGSGWLSDY

SEQ ID NO: 685 - protein, 118 aa, synthetic construct
DVQLVESGGGLVQPGGSLRLSCAASGFPVSNYDMGWFRQAPGKEREFVAAIMGSGGTTYYADSVKGRFTISRDNSKNTVYLQMNSLRPEDTAVYYCASQPEDWYFDLWGRGTLVTVSS

SEQ ID NO: 686 - protein, 8 aa, synthetic construct
GFPVSNYD

SEQ ID NO: 687 - skipped

SEQ ID NO: 688 - protein, 11 aa, synthetic construct
ASQPEDWYFDL

SEQ ID NO: 689 - protein, 122 aa, synthetic construct
EVQLVESGGGLVQPGGSLRLSCAASGFNFGDHAMGWFRQAPGKGRELVAAISSGSGATYYPDSVEGRFTISRDNAKRMVYLQMNSLKSEDTAVYYCARLPVVVTGDGAFDIWGQGTMVTVSS

SEQ ID NO: 690 - protein, 8 aa, synthetic construct
GFNFGDHA

SEQ ID NO: 691 - protein, 8 aa, synthetic construct
ISSGSGAT

SEQ ID NO: 692 - protein, 15 aa, synthetic construct
ARLPVVVTGDGAFDI

SEQ ID NO: 693 - protein, 123 aa, synthetic construct
DVQLVESGGGLVQPGGSLRLSCAASGLPFSDYSMGWFRQAPGKEREFVAGISGSSITTYYADSVKGRFTISRDNSKNTLYLQMNSLRPEDTAVYYCATSGYSYSAGGMDVWGQGTTVTVSSPP

SEQ ID NO: 694 - protein, 123 aa, synthetic construct
DVQLVESGGGLVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSSPP

SEQ ID NO: 695 - protein, 121 aa, synthetic construct
DVQLVESGGGEVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSS

SEQ ID NO: 696 - protein, 123 aa, synthetic construct
DVQLVESGGGEVQPGGSLRLSCAASGLPFSDYSMGWFRQAPGKEREFVAGISGSSITTYYADSVKGRFTISRDNSKNTLYLQMNSLRPEDTAVYYCATSGYSYVAGGMDVWGQGTTVTVSSPP

SEQ ID NO: 697 - protein, 123 aa, synthetic construct
DVQLVESGGGEVQPGGSLRLSCAASGLPFSDYSMGWFRQAPGKEREFVAGISGSSSTTYYADSVKGRFTISRDNSKNTLYLQMNSLRPEDTAVYYCATSGYSYVAGGMDVWGQGTTVTVSSPP

SEQ ID NO: 698 - protein, 123 aa, synthetic construct
DVQLVESGGGEVQPGGSLRLSCAASGLPFSDYSMGWFRQAPGKEREFVAGISGSSITTYYADSVKGRFTISRDNSKNTLYLQMNSLRPEDTAVYYCATSGYSYSAGGMDVWGQGTTVTVSSPP

SEQ ID NO: 699 - protein, 8 aa, synthetic construct
ISGSSSTT

SEQ ID NO: 700 - protein, 6 aa, synthetic construct
GDGSIN

SEQ ID NO: 701 - protein, 133 aa, Homo sapiens [sequence omitted]

SEQ ID NO: 702 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 703 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 704 - protein, 133 aa, synthetic construct
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 705 - protein, 133 aa, synthetic construct
APASSSTKKTQLQLEHLLLDLQMILNGINNHKNPRLTRMLTFKFYMPKKATELKHLQCLEEELKPLEEVLNLAPSKNFHLRPRDLISDINVIVLELKGSETTFMCEYADETATIVEFLNRWITFAQSIISTLT

SEQ ID NO: 706 - protein, 129 aa, synthetic construct
APASSSTKKTQLQLEHLLLDLQMILNGINNYKNPKLTRMLTFKFYMPKKATELKHLQCLEEELKPLEEVLNLAGDASIHDLISRINVIVLELKGSETTFMCEYADETATIVEFLNRWITFSQSIIRTLT

SEQ ID NO: 707 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 708 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 709 - protein, 133 aa, synthetic construct
APASSSTKKTQLQLEHLLLVLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 710 - protein, 133 aa, synthetic construct
APASSSTKKTQLQLESLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 711 - protein, 133 aa, synthetic construct
APASSSTKKTQLQLEHLLLVLQMILNGINNYKNPKLTRMLTFKFYMPKKATELKHLQCLEEELKPLEEVLNLAQSKNFHLRPRDLISNINAIVLELKGSETTFMCEYADETATIVEFLNRWITFSQSIISTLT

SEQ ID NO: 712 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 713 - protein, 133 aa, synthetic construct
APASSSTKKTQLQLSHLLLDLTMILNGINNYKNPKLTRMLTFKFYMPKKATELKHLQCLEEELKPLEEVLNLAQSKNFHLRPRDLISNINLIVLELKGSETTFMCEYADETATIVEFLNRWITFSQSIISTLT

SEQ ID NO: 714 - protein, 133 aa, synthetic construct
APASSSTKKTQLQLSHLLLDLTMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 715 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSTSIISTLT

SEQ ID NO: 716 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIASTLT

SEQ ID NO: 717 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSILSTLT

SEQ ID NO: 718 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIASTLT

SEQ ID NO: 719 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSILSTLT

SEQ ID NO: 720 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIKSTLT

SEQ ID NO: 721 - protein, 133 aa, synthetic construct
APASSSTKKTQLQLSSLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 722 - protein, 133 aa, synthetic construct
APASSSTKKTQLQLEHLLLDLTMILNGINNYKNPKLTRMLTFKFYMPKKATELKHLQCLEEELKPLEEVLNLAQSKNFHLRPRDLISDINVIVLSLKGSETTFMCEYADETATIVEFLNRWITFSQSIISTLT

SEQ ID NO: 723 - protein, 133 aa, synthetic construct
APASSSTKKTQLQLEHLLLDLTMILNGINNYKNPKLTRMLTFKFYMPKKATELKHLQCLEEELKPLEEVLNLAQSKNFHLRPRDLIADINVIVLELKGSETTFMCEYADETATIVEFLNRWITFSQSIISTLT

SEQ ID NO: 724 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRKLISDINVIVLELKGSETTFMCEYADETATIVEFLNRWITFSQSIISTLT

SEQ ID NO: 725 - protein, 133 aa, synthetic construct
APASSSTKKTQLQLSHLLLDLTMILNGINNYKNPKLTRMLTFKFYMPKKATELKHLQCLEEELKPLEEVLNLAQSKNFHLRPRDLIADINVIVLELKGSETTFMCEYADETATIVEFLNRWITFSQSIISTLT

SEQ ID NO: 726 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSTSIISTLT

SEQ ID NO: 727 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIIRTLT

SEQ ID NO: 728 - protein, 129 aa, synthetic construct
APASSSTKKTQLQLEHLLLDLQMILNGINNYKNPKLTRMLTAKFYMPKKATELKHLQCLEEELKPLEEVLNLAGDASIHDLISRINVIVLELKGSETTFMCEYADETATIVEFLNRWITFSQSIIRTLT

SEQ ID NO: 729 - protein, 129 aa, synthetic construct
APASSSTKKTQLQLEHLLLDLQMILNGINNYKNPKLTRMLTFKFYMPKKATELKHLQCLEEELKPLEEVLNLAGDGSINDLISRINVIVLELKGSETTFMCEYADETATIVEFLNRWITFSQSIIRTLT

SEQ ID NO: 730 - protein, 133 aa, synthetic construct
APASSSTKKTQLQLEHLLLDLQMILNGINNYKNPKLTRMLTFKFYMPKKATELKHLQCLEEELKPLEEVLNLAQSANFHLSPRDLISRINVIVLELKGSETTFMCEYADETATIVEFLNRWITFSQSIIRTLT

SEQ ID NO: 731 - protein, 133 aa, synthetic construct
APASSSTKKTQLQLEHLLLDLQMILNGINNYKNPKLTRMLTFKFYMPKKATELKHLQCLEEELKPLEEVLNLAQSANFHLSPSDLISRINVIVLELKGSETTFMCEYADETATIVEFLNRWITFSQSIIRTLT

SEQ ID NO: 732 - protein, 127 aa, synthetic construct
APASSSTKKTQLQLEHLLLDLQMILNGIQSGHMLTFKFYMPKKATELKHLQCLEEELKPLEEVLNLAQSKNFHLRPRDLISRINVIVLELKGSETTFMCEYADETATIVEFLNRWITFSQSIIRTLT

SEQ ID NO: 733 - protein, 133 aa, synthetic construct
APASSSTKKTQLQLEHLLLDLQMILNGINNYSNPELTAMLTFKFYMPKKATELKHLQCLEEELKPLEEVLNLAQSKNFHLRPRDLISRINVIVLELKGSETTFMCEYADETATIVEFLNRWITFSQSIIRTLT

SEQ ID NO: 734 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISRINVLVLELKGSETTFMCEYADETATIVEFLNRWITFSQSIIRTLT

SEQ ID NO: 735 - protein, 133 aa, synthetic construct
APASSSTKKTQLQLSHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWIAFSQSIASTLT

SEQ ID NO: 736 - protein, 133 aa, synthetic construct
APASSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWIAFSQSIISTLT

SEQ ID NO: 737 - protein, 133 aa, synthetic construct
APASSSTKKTQAQLSHLLADLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 738 - protein, 133 aa, synthetic construct
APASSSTKKTQAQLSHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 739 - protein, 133 aa, synthetic construct
APASSSTKKTQYQLSHLLADLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 740 - protein, 133 aa, synthetic construct
APASSSTKKTQYQLEHLLDDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 741 - protein, 133 aa, synthetic construct
APASSSTKKTQLQLDHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 742 - protein, 133 aa, synthetic construct
APASSSTKKTQAQLSHLLADLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 743 - protein, 133 aa, synthetic construct
APASSSTKKTQAQLSHLLDDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 744 - protein, 133 aa, synthetic construct
APASSSTKKTQYQLSHLLADLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 745 - protein, 133 aa, synthetic construct
APASSSTKKTQYQLEHLLDDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 746 - protein, 133 aa, synthetic construct
APASSSTKKTQVQLEHLLVDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 747 - protein, 133 aa, synthetic construct
APASSSTKKTQAQLEHLLADLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 748 - protein, 133 aa, synthetic construct
APASSSTKKTQYQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 749 - protein, 133 aa, synthetic construct
APASSSTKKTQLQLEHLLDDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 750 - protein, 133 aa, synthetic construct
APASSSTKKTQLQLEHLLLDLQMILNGINNHKNPRLTRMLTFKFYMPKKATELKHLQCLEEELKPLEEVLNLAPSKNFHLRPRDLISDINVIVLELKGSETTFMCEYADETATIVEFLNRWITFATSIISTLT

SEQ ID NO: 751 - protein, 129 aa, synthetic construct
APASSSTKKTQLQLEHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSTSIISTLT

SEQ ID NO: 752 - protein, 129 aa, synthetic construct
APASSSTKKTQLQLEHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIKSTLT

SEQ ID NO: 753 - protein, 129 aa, synthetic construct
APASSSTKKTQLQLSHLLLDLQMILNGINNYKNPKLTRMLTFKFYMPKKATELKHLQCLEEELKPLEEVLNLAGDGSINDLISDINVIVLELKGSETTFMCEYADETATIVEFLNRWIAFSQSIASTLT

SEQ ID NO: 754 - protein, 129 aa, synthetic construct
APASSSTKKTQLQLDHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 755 - protein, 129 aa, synthetic construct
APASSSTKKTQAQLSHLLADLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 756 - protein, 129 aa, synthetic construct
APASSSTKKTQYQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 757 - protein, 129 aa, synthetic construct
APASSSTKKTQAQLEHLLADLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 758 - protein, 129 aa, synthetic construct
APASSSTKKTQLQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 759 - protein, 133 aa, synthetic construct
APASSSTKKTQLQLEHLLLDLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSTSIISTLT

SEQ ID NO: 760 - protein, 133 aa, synthetic construct
APASSSTKKTQLQLEHLLLDLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIKSTLT

SEQ ID NO: 761 - protein, 133 aa, synthetic construct
APASSSTKKTQLQLSHLLLDLQMILNGINNYKNPKLTRMLTFKFYMPKKATELKHLQCLEEELKPLEEVLNLAQSANFHLSPRDLISDINVIVLELKGSETTFMCEYADETATIVEFLNRWIAFSQSIASTLT

SEQ ID NO: 762 - protein, 133 aa, synthetic construct
APASSSTKKTQLQLDHLLLDLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIISTLT

SEQ ID NO: 763 - protein, 133 aa, synthetic construct
APASSSTKKTQAQLSHLLADLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIISTLT

SEQ ID NO: 764 - protein, 133 aa, synthetic construct
APASSSTKKTQYQLEHLLDDLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIISTLT

SEQ ID NO: 765 - protein, 133 aa, synthetic construct
APASSSTKKTQAQLEHLLADLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIISTLT

SEQ ID NO: 766 - protein, 133 aa, synthetic construct
APASSSTKKTQLQLEHLLDDLQMILNGINNYKNPKLTRMLTFKFYMPKKATELKHLQCLEEELKPLEEVLNLAQSANFHLSPRDLISDINVIVLELKGSETTFMCEYADETATIVEFLNRWITFSQSIISTLT

SEQ ID NO: 767 - protein, 133 aa, synthetic construct
APASSSTKKTQLQLEHLLLDLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSTSIISTLT

SEQ ID NO: 768 - protein, 133 aa, synthetic construct
APASSSTKKTQLQLEHLLLDLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIKSTLT

SEQ ID NO: 769 - protein, 133 aa, synthetic construct
APASSSTKKTQLQLSHLLLDLQMILNGINNYSNPELTRMLTFKFYMPKKATELKHLQCLEEELKPLEEVLNLAQSKNFHLRPRDLISDINVIVLELKGSETTFMCEYADETATIVEFLNRWIAFSQSIASTLT

SEQ ID NO: 770 - protein, 133 aa, synthetic construct
APASSSTKKTQLQLDHLLLDLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 771 - protein, 133 aa, synthetic construct
APASSSTKKTQAQLSHLLADLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 772 - protein, 133 aa, synthetic construct
APASSSTKKTQYQLEHLLDDLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 773 - protein, 133 aa, synthetic construct
APASSSTKKTQAQLEHLLADLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 774 - protein, 133 aa, synthetic construct
APASSSTKKTQLQLEHLLDDLQMILNGINNYSNPELTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 775 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNHKNPRLTRXLXFKFYXPKKATELKHLQCLEEELKPLEEVLXLAPSKNFHLRPRDLISDINVIVLELKGSETTFXCEYADETATIVEFLNRWITFSQSIISTLT

SEQ ID NO: 776 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXFKFYXPKKATELKHLQCLEEELKPLEEVLXLAQSKNFHLRPRDLISRINVIVLELKGSETTFXCEYADETATIVEFLNRWITFSQSIIRTLT

SEQ ID NO: 777 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 88: note = 4-carboxy-phenylalanine
  SITE 104: note = Homoserine
  SITE 130: note = Ornithine
APTSSSTKKTQLQLEHLLLDLQXILNGINNYKNPKLTRXLXFKFYXPKKATELKHLQCLEEELKPLEEVLXLAQSKNFHLRPRDLISXINVIVLELKGSETTFXCEYADETATIVEFLNRWITFSQSIIXTLT

SEQ ID NO: 778 - protein, 132 aa, synthetic construct
  SITE 16: note = 2,4-diaminobutyric acid
  SITE 22: note = Norleucine
  SITE 38: note = Norleucine
  SITE 40: note = Homoserine
  SITE 45: note = Norleucine
  SITE 70: note = Homoserine
  SITE 87: note = 4-carboxy-phenylalanine
  SITE 103: note = Homoserine
  SITE 125: note = Ornithine
  SITE 129: note = Ornithine
APTSSSTKKTQLQLEXLLDLQXILNGINNYKNPKLTRXLXFKFYXPKKATELKHLQCLEEELKPLEEVLXLAQSKNFHLRPRDLISXINVIVLELKGSETTFXCEYADETATIVEFLNRWITFSXSIIXTLT

SEQ ID NO: 779 - protein, 133 aa, synthetic construct
  SITE 16: note = 2,4-diaminobutyric acid
  SITE 23: note = Norleucine
  SITE 39: note = Norleucine
  SITE 41: note = Homoserine
  SITE 42: note = O-benzyl-tyrosine
  SITE 46: note = Norleucine
  SITE 71: note = Homoserine
  SITE 88: note = 4-carboxy-phenylalanine
  SITE 104: note = Homoserine
  SITE 126: note = Ornithine
  SITE 130: note = Ornithine
APTSSSTKKTQLQLEXLLLDLQXILNGINNYKNPKLTRXLXXKFYXPKKATELKHLQCLEEELKPLEEVLXLAQSKNFHLRPRDLISXINVIVLELKGSETTFXCEYADETATIVEFLNRWITFSXSIIXTLT

SEQ ID NO: 780 - protein, 133 aa, synthetic construct
  SITE 16: note = 2,4-diaminobutyric acid
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
  SITE 126: note = Ornithine
  SITE 130: note = Ornithine
APTSSSTKKTQLQLEXLLLDLQXILNGINNYKNPKLTRXLXFKFYXPKKATELKHLQCLEEELKPLEEVLXLAQSKNFHLRPRDLISNINVIVLELKGSETTFXCEYADETATIVEFLNRWITFSXSIIXTLT

SEQ ID NO: 781 - protein, 133 aa, synthetic construct
  SITE 16: note = 2,4-diaminobutyric acid
  SITE 23: note = Norleucine
  SITE 39: note = Norleucine
  SITE 41: note = Homoserine
  SITE 46: note = Norleucine
  SITE 71: note = Homoserine
  SITE 88: note = 4-carboxy-phenylalanine
  SITE 104: note = Homoserine
  SITE 130: note = Ornithine
APTSSSTKKTQLQLEXLLLDLQXILNGINNYKNPKLTRXLXFKFYXPKKATELKHLQCLEEELKPLEEVLXLAQSKNFHLRPRDLISXINVIVLELKGSETTFXCEYADETATIVEFLNRWITFSQSIIXTLT

SEQ ID NO: 782 - protein, 129 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 100: note = Homoserine
APTSSSTKKTQLQLEHLLLDLQXILNGINNYKNPKLTRXLXFKFYXPKKATELKHLQCLEEELKPLEEVLXLAQSKNFHDLISRINVIVLELKGSETTFXCEYADETATIVEFLNRWITFSQSIIRTLT

SEQ ID NO: 783 - protein, 129 aa, synthetic construct
APTSSSTKKTQLQLEHLLLDLQMILNGINNYKNPKLTRMLTFKFYMPKKATELKHLQCLEEELKPLEEVLNLAGDGSINDLISNINVIVLELKGSETTFMCEYADETATIVEFLNRWITFCQSIISTLT

SEQ ID NO: 784 - protein, 73 aa, synthetic construct
APTSSSTKKTQLQLEHLLLDLQMILNGINNYKNPKLTRMLTFKFYMPKKATELKHLQCLEEELKPLEEVLNLA

SEQ ID NO: 785 - protein, 50 aa, synthetic construct
DLISNINVIVLELKGSETTFMCEYADETATIVEFLNRWITFCQSIISTLT

SEQ ID NO: 786 - protein, 6 aa, synthetic construct
GDASIH

SEQ ID NO: 787 - protein, 4 aa, synthetic construct
QSGH

SEQ ID NO: 788-800 - skipped (13 entries)

SEQ ID NO: 801 - protein, 372 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 802 - protein, 365 aa, synthetic construct
APASSSTKKTQLQLDHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 803 - protein, 365 aa, synthetic construct
APASSSTKKTQLQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 804 - protein, 597 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 805 - protein, 597 aa, synthetic construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLDHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLT

SEQ ID NO: 806 - protein, 511 aa, synthetic construct
APASSSTKKTQLQLDHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 807 - protein, 511 aa, synthetic construct
APASSSTKKTQLQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 808 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 809 - protein, 513 aa, synthetic construct
APASSSTKKTQLQLDHLLLDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

SEQ ID NO: 810 - protein, 513 aa, synthetic construct
APASSSTKKTQLQLEHLLDDLQMILNGINNYKNPKLTRMLTFKFYMPKKATELKHLQCLEEELKPLEEVLNLAGDGSINDLISDINVIVLELKGSETTFMCEYADETATIVEFLNRWITFSQSIISTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSPP

(19) United States
(12) Patent Application Publication
(10) Pub. No.: US 2026/0184792 A1
(43) Pub. Date: Jul. 2, 2026

(54) TRISPECIFIC COMPOSITIONS COMPRISING IL-2, VEGF BINDING DOMAINS, AND PD-1 BINDING DOMAINS

(71) Applicant: Bright Peak Therapeutics AG, Allschwil (CH)

(72) Inventors: Vijaya Raghavan PATTABIRAMAN, Volketswil (CH); Bertolt KREFT, Kleinmachnow (DE); Arnaud GOEPFERT, Sierentz (FR); Kea MARTIN, Istein (DE); Nina RESCHKE, Birsfelden (CH); Olivier BORNERT, Baldenheim (FR)

(21) Appl. No.: 19/569,819

(22) Filed: Mar. 17, 2026

Related U.S. Application Data
(63) Continuation of application No. 19/359,286, filed on Oct. 15, 2025.
(60) Provisional application No. 63/708,077, filed on Oct. 16, 2024.
(60) Provisional application No. 63/739,330, filed on Dec. 27, 2024.

(51) Int. Cl. C07K 16/28; A61K 39/00; A61P 35/00; C07K 14/55; C07K 16/22

(52) U.S. Cl. CPC C07K 16/2818; A61P 35/00; C07K 14/55; C07K 16/22; A61K 2039/505; C07K 2317/31; C07K 2317/35; C07K 2317/55; C07K 2317/565; C07K 2317/569; C07K 2317/622; C07K 2317/92; C07K 2319/00

(57) ABSTRACT

The present disclosure relates anti-PD-1/anti-VEGF immunocytokines, particularly with IL-2. Also described herein are bispecific anti-PD-1 and anti-VEGF constructs. IL-2 polypeptides are also provided herein. Also provided herein are pharmaceutical compositions and methods of treatment using the aforementioned compositions.

### CROSS REFERENCE

This application is continuation of claims the benefit of U.S. patent application Ser. No. 19/359,286, filed Oct. 15, 2025, which claims the benefit of U.S. Provisional Application No. 63/708,077, filed Oct. 16, 2024, and U.S. Provisional Application No. 63/739,330, filed Dec. 27, 2024, which applications are incorporated by reference in their entirety.

### SEQUENCE LISTING

The instant application contains a Sequence Listing which has been submitted electronically in XML file format and is hereby incorporated by reference in its entirety. Said XML copy, created on Mar. 16, 2026, is named 56146-746_301_SL.xml and is 764,075 bytes in size.

### BRIEF SUMMARY

Described herein are multifunctional immunocytokine compositions which comprise a PD-1 binding domain, a VEGFA binding domain, and a cytokine. In some embodiments, the cytokine is an IL-2 polypeptide. Such compositions are useful in the treatment of diseases and disorders such as cancer.

In some embodiments of a multifunctional immunocytokine composition, the VEGFA binding domain and PD-1 binding domain and/or IL-2 polypeptide exhibit cooperative behavior such that binding of the VEGFA binding domain to VEGFA (e.g., VEGF dimers) results in enhanced anti-PD-1 activity and/or enhanced IL-2 activity. In some embodiments, this cooperative behavior results from a non-covalent multimerization of the immunocytokine composition when the VEGFA binding domain binds to VEGFA. For example, in some embodiments, a first molecule of the multifunctional immunocytokine can bind to a monomer of a VEGF dimer in situ and a second molecule of the multifunctional immunocytokine can bind to the other monomer of the VEGF dimer, thus forming, in essence, a non-covalent multimer of the multifunctional immunocytokine. Once formed, the multimer can exhibit enhanced anti-PD-1 and/or enhanced IL-2 activity (e.g., by avidity-like effects) compared to the multifunctional immunocytokine without the presence of VEGFA. Thus, in some embodiments, multifunctional immunocytokines of the instant disclosure can provide for delivery of PD-1 and/or IL-2 to a subject with increased therapeutic index by optimizing the activity in tumor microenvironments where VEGFA is present.

In some embodiments, a multifunctional immunocytokine of the instant disclosure utilizes an IL-2 polypeptide with reduced activity compared to wild type IL-2 (SEQ ID NO: 701, human IL-2). In some embodiments, use of such an IL-2 polypeptide allows for reduced off-target effects as the IL-2 can be sufficiently active due to the targeting nature of the other components of the immunocytokine and/or the multimerization effects described supra, yet will be less active outside of target tissue, thereby enhancing therapeutic index.

In some embodiments, an IL-2 polypeptide of the instant disclosure exhibits reduced binding to heparin compared to other IL-2 polypeptides or to wild type IL-2. In some embodiments, the reduced binding to heparin can impart favorable characteristics to the immunocytokine composition, such as enhanced PK, half-life, bioavailability, and biodistribution due to the prevention of accumulation of the immunocytokine composition in heparin rich tissues.

Also described herein are novel anti-VEGFA and PD-1 binding domains with certain advantages over those otherwise known. Such binding domains in some instances have optimal properties for inclusion in an immunocytokine composition of the instant disclosure.

Further provided herein are methods of treating cancer and other disease with the aforementioned compositions, as well as pharmaceutical compositions comprising the same.

In an aspect, the present disclosure provides a composition, comprising: a) a first binding domain targeting programmed cell death protein 1 (PD-1); b) a second binding domain targeting vascular endothelial growth factor A (VEGFA); and c) a cytokine, wherein each of the first binding domain, the second binding domain, and the cytokine are in covalent association.

In an aspect, the present disclosure provides a multifunctional immunocytokine composition, comprising: a) a first binding domain targeting programmed cell death protein 1 (PD-1); b) a second binding domain targeting vascular endothelial growth factor A (VEGFA); and c) a cytokine, wherein each of the first binding domain, the second binding domain, and the cytokine are in covalent association. In some embodiments, the cytokine is selected from an interleukin, a TNF family cytokine, an interferon, a TGF-b family cytokine, and a chemokine.

In some embodiments, the cytokine is an IL-2 polypeptide. In some embodiments, the IL-2 polypeptide comprises an amino acid sequence having at least 80%, 85%, 90%, 95%, 96%, 97%, 98%, 99% or 100% a sequence identity to wild type IL-2 (SEQ ID NO: 701). In some embodiments, the IL-2 polypeptide has reduced affinity for the IL-2 receptor beta subunit compared to the IL-2 of SEQ ID NO: 701. In some embodiments, the IL-2 polypeptide exhibits a reduced ability to signal through the IL-2 receptor beta/gamma complex compared to the IL-2 of SEQ ID NO: 701. In some embodiments, the IL-2 polypeptide retains the ability to bind to the IL-2 receptor alpha subunit and exhibits a diminished ability to bind to the IL-2 receptor beta or gamma subunits relative to SEQ ID NO: 701. In some embodiments, the IL-2 polypeptide has reduced affinity for heparin compared to the IL-2 of SEQ ID NO: 701. In some embodiments, the IL-2 polypeptide comprises a modified B′C′ loop region, wherein the modified B′C′ loop region comprises a deletion of one or more amino acids of the B′C′ loop region between amino acids 73 and 84 of the IL-2 polypeptide and insertion of an exogenous peptide into the B′C′ loop region, wherein residue position numbering is based on SEQ ID NO: 701 as a reference sequence. In some embodiments, the inserted peptide comprises the sequence GDGSIN (SE ID NO: 700). In some embodiments, the inserted peptide consists of the sequence GDGSIN. In some embodiments, the deletion of one or more amino acids of the B′C′ loop region comprises a deletion of 1, 2, 3, 4, 5, 6, 7, 8, 9 or 10 amino acids between amino acids 73 and 84 of the IL-2 polypeptide. In some embodiments, the deletion of one or more amino acids of the B′C′ loop region comprises a deletion of each of amino acids 74-83 of the IL-2 polypeptide. In some embodiments, the IL-2 polypeptide comprises a peptide sequence having at least 80%, 85%, 90%, 95%, 96%, 97%, 98%, 99%, or 100% sequence identity to amino acids 1-73 of SEQ ID NO: 701 and a peptide sequence having at least 80%, 85%, 90%, 95%, 96%, 97%, 98%, 99%, or 100% sequence identity to amino acids 84-133 of SEQ ID NO: 501. In some embodiments, the IL-2 polypeptide comprises a peptide sequence having at least 80%, 85%, 90%, 95%, 96%, 97%, 98%, 99%, or 100% identity to SEQ ID NO: 783. In some embodiments, the IL-2 polypeptide comprises a K76A or R81S substitution, or both. In some embodiments, the IL-2 polypeptide comprises polypeptide comprises an amino acid substitution at residue N88. In some embodiments, the IL-2 polypeptide comprises an N88D substitution. In some embodiments, the IL-2 polypeptide comprises a substitution at any one of residues L12, E15, L19, T123, Q126, or I129. In some embodiments, the IL-2 polypeptide comprises one or more substitutions selected from L12A, L12Y, E15D, E15S, L19A, L19D, T123A, Q126T, I129A, and I129K. In some embodiments, the IL-2 polypeptide comprises any one of the following sets of substitutions: Q126T; I129K; I129A, E15S, T123A; E15D; L12A, L19A, E15S; L12Y, L19D; L12A, L19A; or L19D. In some embodiments, the IL-2 polypeptide comprises an E15D or an L19D substitution. In some embodiments, the IL-2 polypeptide comprises a T3A substitution. In some embodiments, the IL-2 polypeptide comprises a C125S substitution. In some embodiments, the composition comprises the sequence set forth in any one of SEQ ID NOs: 702-783. In some embodiments, the IL-2 polypeptide is in covalent association via a fusion of the IL-2 polypeptide to the portion of the composition to which it is attached. In some embodiments, the IL-2 polypeptide is fused via its C-terminus.

In some embodiments, the first binding domain targeting programmed cell death protein 1 (PD-1) is capable of disrupting the interaction of PD-1 with programmed cell death ligand 1 (PD-L1). In some embodiments, the first binding domain is an antigen binding fragment derived from an antibody. In some embodiments, the first binding domain comprises a heavy chain variable domain (VH) comprising a heavy chain first complementary determining region (VH CDR1), a heavy chain second complementary determining region (VH CDR2), and a heavy chain third complementary determining region (VH CDR3). In some embodiments, the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. In some embodiments, the VH comprises a set of VH CDR1, VH CDR2, and VH CDR3 derived from an antibody in Table 1A, 1B, or 1C. In some embodiments, the VH comprise an amino acid sequence of a VH set forth in Table 1A, 1B, or 1C.

In some embodiments, the first binding domain is a VHH. In some embodiments, the first binding domain comprises: a) a VH CDR1 sequence of SEQ ID NO: 2; a VH CDR2 sequence of SEQ ID NO: 3; and a VH CDR3 sequence of SEQ ID NO: 4; b) a VH CDR1 sequence of SEQ ID NO: 6; a VH CDR2 sequence of SEQ ID NO: 7; and a VH CDR3 sequence of SEQ ID NO: 8; c) a VH CDR1 sequence of SEQ ID NO: 14; a VH CDR2 sequence of SEQ ID NO: 15; and a VH CDR3 sequence of CDR3 SEQ ID NO: 16; d) a VH CDR1 sequence of SEQ ID NO: 18; a VH CDR2 sequence of SEQ ID NO: 19; and a VH CDR3 sequence of CDR 3 of SEQ ID NO: 20; e) a VH CDR1 sequence of SEQ ID NO: 288, a VH CDR2 sequence of SEQ ID NO: 289, and a VH CDR3 sequence of SEQ ID NO: 290, or f) a VH CDR1, VH CDR2, and VHCDR3 of a VHH provided in Table 1C. In some embodiments, the VHH comprises an amino acid sequence having at least 80%, 85%, 90%, 95%, 96%, 97%, 98%, 99%, or 100% identity to any one of SEQ ID NOs: 1, 5, 13, 17, 287, or that of a VHH provided in Table 1C.

In some embodiments, the VH comprises: a) a VH CDR1 sequence of NYYMY (SEQ ID NO: 80), a VH CDR2 sequence of GINPSNGGTNFNEKFKN (SEQ ID NO: 81), and a VH CDR3 sequence of RDYRFDMGFDY (SEQ ID NO: 82); b) a VH CDR1 sequence of NSGMH (SEQ ID NO:86), a VH CDR2 sequence of VIWYDGSKRYYADSVKG (SEQ ID NO: 87), and a VH CDR3 sequence of NDDY (SEQ ID NO: 88); or c) a VH CDR1 sequence of GYTFTSYYMY (SEQ ID NO: 113), a VH CDR2 sequence of GVNPSNGGTNFNEKFKS (SEQ ID NO: 114), and a VH CDR3 sequence of RDYRYDMGFDY (SEQ ID NO: 115). In some embodiments, the VH comprises an amino acid sequence having at least 80%, 85%, 90%, 95%, 96%, 97%, 98%, 99%, or 100% identity to any one of SEQ ID NOs: 48, 50, or 76.

In some embodiments, the first binding domain comprises a light chain variable domain (VL) comprising a light chain first complementary determining region (VL CDR1), a light chain second complementary determining region (VL CDR2), and a light chain third complementary determining region (VL CDR3). In some embodiments, the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the first binding domain is comprised. In some embodiments, the VL comprises a set of VL CDR1, VL CDR2, and VL CDR3 derived from an antibody in Table 1A or 1B. In some embodiments, the VL comprises: a) a VL CDR1 sequence of RASKGVSTSGYSYLH (SEQ ID NO: 83), a VL CDR2 sequence of LASYLES (SEQ ID NO: 84), and a VL CDR3 sequence of QHSRDLPLT (SEQ ID NO: 85); b) a VL CDR1 sequence of RASQSVSSYLA (SEQ ID NO: 89), a VL CDR2 sequence of DASNRAT (SEQ ID NO: 90), and a VL CDR3 sequence of QQSSNWPRT (SEQ ID NO: 91); or c) a VL CDR1 sequence of RASKGVSTSGYSYLH (SEQ ID NO: 83), a VL CDR2 sequence of LASYLE (SEQ ID NO: 117), and a VL CDR3 sequence of QHSRELPLT (SEQ ID NO: 118). In some embodiments, the VL comprises an amino acid sequence having at least 80%, 85%, 90%, 95%, 96%, 97%, 98%, 99%, or 100% identity to any one of SEQ ID NOs: 49, 51, or 77.

In some embodiments, the first binding domain comprises: a) a VH having a VH CDR1 sequence of NYYMY (SEQ ID NO: 80), a VH CDR2 sequence of GINPSNGGTNFNEKFKN (SEQ ID NO: 81), and a VH CDR3 sequence of RDYRFDMGFDY (SEQ ID NO: 82), and a VL having a VL CDR1 sequence of RASKGVSTSGYSYLH (SEQ ID NO: 83), a VL CDR2 sequence of LASYLES (SEQ ID NO: 84), and a VL CDR3 sequence of QHSRDLPLT (SEQ ID NO: 85); or b) a VH having a VH CDR1 sequence of NSGMH (SEQ ID NO: 86), a VH CDR2 sequence of VIWYDGSKRYYADSVKG (SEQ ID NO: 87), and a VH CDR3 sequence of NDDY (SEQ ID NO: 88), and a VL having a VL CDR1 sequence of RASQSVSSYLA (SEQ ID NO: 89), a VL CDR2 sequence of DASNRAT (SEQ ID NO: 90), and a VL CDR3 sequence of QQSSNWPRT (SEQ ID NO: 91; or c) a VH having a VH CDR1 sequence of GYTFTSYYMY (SEQ ID NO: 113), a VH CDR2 sequence of GVNPSNGGTNFNEKFKS (SEQ ID NO: 114), and a VH CDR3 sequence of RDYRYDMGFDY (SEQ ID NO: 115), and a VL having a VL CDR1 sequence of RASKGVSTSGYSYLH (SEQ ID NO: 83), a VL CDR2 sequence of LASYLE (SEQ ID NO: 117), and a VL CDR3 sequence of QHSRELPLT (SEQ ID NO: 118). In some embodiments, the first binding domain comprises: a) a VH comprising an amino acid sequence amino acid sequence having at least 80%, 85%, 90%, 95%, 96%, 97%, 98%, 99%, or 100% identity to SEQ ID NO: 48 and a VL comprising an amino acid sequence having at least 80%, 85%, 90%, 95%, 96%, 97%, 98%, 99%, or 100% identity to SEQ ID NO: 49; b) a VH comprising an amino acid sequence amino acid sequence having at least 80%, 85%, 90%, 95%, 96%, 97%, 98%, 99%, or 100% identity to SEQ ID NO: 50 and a VL comprising an amino acid sequence having at least 80%, 85%, 90%, 95%, 96%, 97%, 98%, 99%, or 100% identity to SEQ ID NO: 51; or c) a VH comprising an amino acid sequence amino acid sequence having at least 80%, 85%, 90%, 95%, 96%, 97%, 98%, 99%, or 100% identity to SEQ ID NO: 76 and a VL comprising an amino acid sequence having at least 80%, 85%, 90%, 95%, 96%, 97%, 98%, 99%, or 100% identity to SEQ ID NO: 77.

In some embodiments, the first binding domain is an scFv. In some embodiments, the first binding domain is a Fab.

In some embodiments, the second binding domain targeting VEGFA is capable of disrupting the interaction of VEGFA with one or more of its receptors. In some embodiments, the second binding domain is comprised in an antigen binding fragment derived from an antibody.

In some embodiments, the second binding domain comprises a heavy chain variable domain (VH) comprising a heavy chain first complementary determining region (VH CDR1), a heavy chain second complementary determining region (VH CDR2), and a heavy chain third complementary determining region (VH CDR3). In some embodiments, the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. In some embodiments, the VH comprises a set of VH CDR1, VH CDR2, and VH CDR3 derived from an antibody in Table 2A or 2B. In some embodiments, the VH comprises an amino acid sequence of a VH set forth in Table 2A, 2B, or 2C. In some embodiments, the VH comprises a VH CDR1 having a sequence GYTFTNYGMN (SEQ ID NO: 123), a VH CDR2 having a sequence WINTYTGEPTYAADFK (SEQ ID NO: 124), and a VH CDR3 having a sequence YPHYYGSSHWYFDV (SEQ ID NO: 125). In some embodiments, the VH comprises an amino acid sequence having at least 80%, 85%, 90%, 95%, 96%, 97%, 98%, 99%, or 100% identity to SEQ ID NO: 122. In some embodiments, the second binding domain comprises a light chain variable domain (VL) comprising a light chain first complementary determining region (VL CDR1), a light chain second complementary determining region (VL CDR2), and a light chain third complementary determining region (VL CDR3). In some embodiments, the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the second binding domain is comprised. In some embodiments, the VL comprises a set of VL CDR1, VL CDR2, and VL CDR3 derived from an antibody in Table 2A or 2B. In some embodiments, the VL comprises a VL CDR1 having a sequence SASQDISNYLN (SEQ ID NO: 128), a VL CDR2 having a sequence FTSSLHS (SEQ ID NO: 129), and a VL CDR3 having a sequence QQYSTVPWT (SEQ ID NO: 130). In some embodiments, the VL comprises an amino acid sequence having at least 80%, 85%, 90%, 95%, 96%, 97%, 98%, 99%, or 100% identity to SEQ ID NO: 127. In some embodiments, the second binding domain comprises a VH having a VH CDR1 having a sequence GYTFTNYGMN (SEQ ID NO: 123), a VH CDR2 having a sequence WINTYTGEPTYAADFK (SEQ ID NO: 124), and a VH CDR3 having a sequence YPHYYGSSHWYFDV (SEQ ID NO: 125), and a VL having a VL CDR1 having a sequence SASQDISNYLN (SEQ ID NO: 128), a VL CDR2 having a sequence FTSSLHS (SEQ ID NO: 129), and a VL CDR3 having a sequence QQYSTVPWT (SEQ ID NO: 130). In some embodiments, the second binding domain comprises a VH comprising an amino acid sequence having at least 80%, 85%, 90%, 95%, 96%, 97%, 98%, 99%, or 100% sequence identity to SEQ ID NO: 122 and a VL comprising an amino acid sequence having at least 80%, 85%, 90%, 95%, 96%, 97%, 98%, 99%, or 100% sequence identity to SEQ ID NO: 127.

In some embodiments, the second binding domain is an scFv. In some embodiments, the second binding domain is a Fab.

In some embodiments, the second binding domain is a single domain antibody. In some embodiments, the second binding domain is a single domain heavy chain antibody (VHH). In some embodiments, the second binding domain comprises: a) a VH CDR1 sequence of AYPMM (SEQ ID NO: 202), a VH CDR2 sequence of EISPSGSYTYYADSVRG (SEQ ID NO: 203), and a VH CDR3 sequence of DPRKLDY (SEQ ID NO: 204); b) a VH CDR1 sequence of LYDMM (SEQ ID NO: 206), a VH CDR2 sequence of FIGGDGLNTYYADSVKG (SEQ ID NO: 207), and a VH CDR3 sequence of AGTQFDY (SEQ ID NO: 208); c) a VH CDR1 sequence of WYPMW (SEQ ID NO: 210), a VH CDR2 sequence of LIEGQGDRTYYADSVKG (SEQ ID NO: 211), and a VH CDR3 sequence of AGDRTAGSRGNSFDY (SEQ ID NO: 212); d) a VH CDR1 sequence of AYPMM (SEQ ID NO: 202), a VH CDR2 sequence of EISPSGSYTYYADSVKG (SEQ ID NO: 215), and a VH CDR3 sequence of DPRKFDY (SEQ ID NO: 216); or e) a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain comprises an amino acid sequence having at least 80%, 85%, 90%, 95%, 96%, 97%, 98%, 99% or 100% identity to any one of SEQ ID NOs: 200, 201, 205, 209, 213, 217, 221, or 280-284. In some embodiments, the second binding domain comprises a light chain single domain antibody. In some embodiments, the second binding domain comprises a VL CDR1 having the sequence RASQWIGPELS (SEQ ID NO: 223), a VL CDR2 having the sequence HTSILQS (SEQ ID NO: 224), and a VL CDR3 having the sequence QQYMFQPRT (SEQ ID NO: 225). In some embodiments, the second binding domain comprises an amino acid sequence having at least 80%, 85%, 90%, 95&, 96%, 97%, 98%, 99% or 100% identity to SEQ ID NO: 222.

In some embodiments, the second binding domain is an anti-VEGFA anticalin. In some embodiments, the second binding domain comprises an amino acid sequence having at least 80%, 85%, 90%, 95%, 96%, 97%, 98%, 99% or 100% identity to SEQ ID NO: 226.

In some embodiments, the composition comprises multiple copies of the first binding domain, the second binding domain, or both. In some embodiments, the composition comprises multiple copies of the second binding domain. In some embodiments, the composition comprises two copies of the second binding domain. In some embodiments, one of the copies of the second binding domain contains an extension of one or more amino acids on the second binding domain relative to the other copy. In some embodiments, the multiple copies of the second binding domain are present on the same polypeptide chain. In some embodiments, the multiple copies of the second binding domain are present on different polypeptide chains.

In some embodiments, the composition comprises an Fc domain comprising first CH2 and CH3 domains on a first polypeptide chain and second CH2 and CH3 domains on a second polypeptide chain. In some embodiments, the Fc domain is derived from an IgG. In some embodiments, the Fc domain is derived from an IgG1 or IgG4. In some embodiments, the composition comprises a structure of the formula:

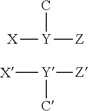

wherein: Y is the first CH2 and CH3 domains; Y′ is the second CH2 and CH3 domains; X and X′ are each independently the first binding domain, the second binding domain, a copy of the first binding domain, a copy of the second binding domain, the cytokine, a copy of the cytokine, a masking polypeptide for the cytokine, or absent; Z and Z′ are each independently the first binding domain, the second binding domain, a copy of the first binding domain, a copy of the second binding domain, the cytokine, a copy of the cytokine, a third binding domain targeting PD-1, or a fourth binding domain targeting VEGFA, a masking polypeptide for the cytokine, or absent. C and C′ are each independently the cytokine, a copy of the cytokine, or absent, wherein C and C′, if present, are attached to a side chain of a residue of the Fc domain via a linker; wherein X, Y, and Z and X′, Y′ and Z′ are depicted in an N-terminal to C-terminal direction; and wherein each of X, X′, Z, and Z′ are independently and optionally connected to Y or Y′ via a peptide linker.

In some embodiments, X is the first binding domain; X′ is a copy of the first binding domain, the cytokine, or absent; one of Z or Z′ is the second binding domain and the other is absent, the cytokine, or a copy of the second binding domain; and one of C or C′ is the cytokine and the other is absent, or both C and C′ are absent. In some embodiments, X′ is the cytokine and C and C′ are both absent. In some embodiments, X′ is the copy of the first binding domain and one of C or C′ is the cytokine. In some embodiments, X′ is the copy of the first binding domain, one of Z or Z′ is the second binding domain and the other is the cytokine, and both C and C′ are absent. In some embodiments: Z is the second binding domain and Z′ is a copy of the second binding domain; Z is the second binding domain and Z′ is absent; or Z is absent and Z′ is the second binding domain. In some embodiments, X is the second binding domain, X′ is a copy of the second binding domain, the cytokine, or absent; one of Z or Z′ is the first binding domain and the other is absent, the cytokine, or a copy of the first binding domain; and one of C or C′ is the cytokine and the other is absent, or both C and C′ are absent. In some embodiments, X′ is the cytokine and C and C′ are both absent. In some embodiments, X′ is a copy of the second binding domain one of C or C′ is the cytokine. In some embodiments, X′ is the copy of the second binding domain, one of Z or Z′ is the second binding domain and the other is the cytokine, and both C and C′ are absent. In some embodiments: Z is the first binding domain and Z′ is a copy of the first binding domain; Z is the first binding domain and Z′ is absent; or Z is absent and Z′ is the first binding domain. In some embodiments, X is the first binding domain, X′ is the second binding domain, one of C or C′ is the cytokine and the other is absent, Z is absent or the third binding domain, and Z′ is absent or the fourth binding domain. In some embodiments: Z is the third binding domain and Z′ is absent; Z is absent and Z′ is the fourth binding domain; or both Z and Z′ are absent. In some embodiments, X is the first binding domain, X′ is the second binding domain, one of Z or Z′ is the cytokine and the other is the masking polypeptide for the cytokine, and both C and C′ are absent. In some embodiments, the masking polypeptide for the cytokine is attached to the Y or Y′ to which it is connected via a cleavable peptide linker. In some embodiments, if the third binding domain is present, the first binding domain comprises a Fab and the third binding domain comprises an scFv, and wherein the Fab and the scFv comprise the same VH and VL. In some embodiments, if the fourth binding domain is present, the second binding domain comprises a Fab and the fourth binding domain comprises an scFv, and wherein the Fab and the scFv comprise the same VH and VL. In some embodiments, X is one of the first or second binding domains and is a Fab, VHH, or scFv. In some embodiments, X is one of the first or second binding domains and is a Fab. In some embodiments, X′ is a copy of X. In some embodiments, one Z or Z′ is one of the first or second binding domains and is an scFv or a VHH, wherein if X is the first binding domain then Z or Z′ is the second binding domain and if X is the second binding domain then Z or Z′ is the first binding domain.

In some embodiments: X is a Fab and the first binding domain, X′ is a copy of the first binding domain, Z is an scFv or VHH and is the second binding domain, Z′ is a copy of the second binding domain, C is the cytokine, and C′ is absent; or X is a Fab and the second binding domain, X′ is a copy of the second binding domain, Z is an scFv or VHH and is the first binding domain, Z′ is a copy of the first binding domain, C is the cytokine, and C′ is absent; or X is a Fab and is the first binding domain, X′ is a Fab and is the second binding domain, Z and Z′ are absent, and C or C′ is the cytokine and the other is absent; X is a Fab and is the first binding domain, X′ is a Fab and is the second binding domain; Z is an scFv or VHH and is the third binding domain, Z′ is absent, and C or C′ is the cytokine and the other is absent; or X is a Fab and is the second binding domain, X′ is a Fab and is the first binding domain, Z is an scFv or VHH and is the fourth binding domain, Z′ is absent, and C or C′ is the cytokine and the other is absent; or X is a Fab and is the first binding domain, X′ is the cytokine, Z is an scFv or VHH and is the second binding domain, Z′ is a copy of the second binding domain, and C and C′ are both absent; or X is a Fab and is the second binding domain, X′ is the cytokine, Z is an scFv or VHH and is the first binding domain, Z′ is a copy of the first binding domain, and C and C′ are both absent; or X is a Fab and is the first binding domain, X′ is an scFv and is the second binding domain; Z and Z′ are both absent, and C or C′ is the cytokine and the other is absent; or X is a Fab and is the second binding domain, X′ is an scFv and is the first binding domain, Z and Z′ are both absent, and C or C′ is the cytokine and the other is absent; or X is a Fab and is the first binding domain, X′ is Fab or ScFv and is the second binding domain, one of Z or Z′ is the cytokine and the other is the masking polypeptide for the cytokine, and C and C′ are both absent; or X is a Fab and is the second binding domain, X′ is a Fab or scFv and is the first binding domain, one of Z or Z′ is the cytokine and the other is the masking polypeptide for the cytokine, and C and C′ are both absent.

In some embodiments, the first binding domain comprises a Fab, and wherein the composition comprises: a) a first polypeptide chain comprising the VL of the first binding domain; b) a second polypeptide chain comprising the VH of the first binding domain; and c) a third polypeptide chain comprising the IL-2 polypeptide.

In some embodiments, the first binding domain comprises: a) a VH having a VH CDR1 sequence of NYYMY (SEQ ID NO: 80), a VH CDR2 sequence of GINPSNGGTNFNEKFKN (SEQ ID NO: 81), and a VH CDR3 sequence of RDYRFDMGFDY (SEQ ID NO: 82), and a VL having a VL CDR1 sequence of RASKGVSTSGYSYLH (SEQ ID NO: 83), a VL CDR2 sequence of LASYLES (SEQ ID NO: 84), and a VL CDR3 sequence of QHSRDLPLT (SEQ ID NO: 85); or b) a VH having a VH CDR1 sequence of NSGMH (SEQ ID NO: 86), a VH CDR2 sequence of VIWYDGSKRYYADSVKG (SEQ ID NO: 87), and a VH CDR3 sequence of NDDY (SEQ ID NO: 88), and a VL having a VL CDR1 sequence of RASQSVSSYLA (SEQ ID NO: 89), a VL CDR2 sequence of DASNRAT (SEQ ID NO: 90), and a VL CDR3 sequence of QQSSNWPRT (SEQ ID NO: 91); or c) a VH having a VH CDR1 sequence of GYTFTSYYMY (SEQ ID NO: 113), a VH CDR2 sequence of GVNPSNGGTNFNEKFKS (SEQ ID NO: 114), and a VH CDR3 sequence of RDYRYDMGFDY (SEQ ID NO: 115), and a VL having a VL CDR1 sequence of RASKGVSTSGYSYLH (SEQ ID NO: 83), a VL CDR2 sequence of LASYLE (SEQ ID NO: 117), and a VL CDR3 sequence of QHSRELPLT (SEQ ID NO: 118). In some embodiments, the first binding domain comprises: a) a VH comprising an amino acid sequence amino acid sequence having at least 80%, 85%, 90%, 95%, 96%, 97%, 98%, 99%, or 100% identity to SEQ ID NO: 48 and a VL comprising an amino acid sequence having at least 80%, 85%, 90%, 95%, 96%, 97%, 98%, 99%, or 100% identity to SEQ ID NO: 49; b) a VH comprising an amino acid sequence amino acid sequence having at least 80%, 85%, 90%, 95%, 96%, 97%, 98%, 99%, or 100% identity to SEQ ID NO: 50 and a VL comprising an amino acid sequence having at least 80%, 85%, 90%, 95%, 96%, 97%, 98%, 99%, or 100% identity to SEQ ID NO: 51; or c) a VH comprising an amino acid sequence amino acid sequence having at least 80%, 85%, 90%, 95%, 96%, 97%, 98%, 99%, or 100% identity to SEQ ID NO: 76 and a VL comprising an amino acid sequence having at least 80%, 85%, 90%, 95%, 96%, 97%, 98%, 99%, or 100% identity to SEQ ID NO: 77. In some embodiments, the first polypeptide chain comprises, in N- to C-terminal direction, the VL and light chain constant region. In some embodiments, the light chain constant region comprises an amino acid having at least 80%, 85%, 90%, 95%, 96%, 97%, 98%, 99%, or 100% sequence identity to SEQ ID NO: 276. In some embodiments, the first polypeptide chain comprises the sequence

(SEQ ID NO: 47)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In some embodiments, the second polypeptide chain comprises, in an N-terminal to C-terminal direction, the VH of the Fab of the first binding domain and an antibody constant region. In some embodiments, the antibody constant region is an IgG1 or IgG4 constant region. In some embodiments, the antibody constant region comprises, in an N-terminal to C-terminal direction, a CH1 domain, a hinge region, a CH2 domain, and a CH3 domain. In some embodiments: a) the CH1 domain comprises an amino acid sequence having at least 80%, 85%, 90%, 95%, 96%, 97%, 98%, 99%, or 100% identity to any one of SEQ ID NOs: 272-275; b) the hinge region comprises an amino acid sequence having at least 80%, 85%, 90%, 95%, 96%, 97%, 98%, 99%, or 100% identity to any one of SEQ ID NOs 265-271; and/or c) the CH2 and CH3 domains together comprise an amino acid sequence having at least 80%, 85%, 90%, 95%, 96%, 97%, 98%, 99%, or 100% identity to any one of SEQ ID NOs 229-234. In some embodiments, the second polypeptide chain comprises, in an N-terminal to C-terminal direction, the VH of the Fab, the antibody constant region, an optional peptide linker, and the second binding domain. In some embodiments, the optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the second binding domain is a VHH. In some embodiments, the VHH comprises a two proline peptide on its C-terminus. In some embodiments, the VHH comprises a) a VH CDR1 sequence of AYPMM (SEQ ID NO: 202), a VH CDR2 sequence of EISPSGSYTYYADSVRG (SEQ ID NO: 203), and a VH CDR3 sequence of DPRKLDY (SEQ ID NO: 204); b) a VH CDR1 sequence of LYDMM (SEQ ID NO: 206), a VH CDR2 sequence of FIGGDGLNTYYADSVKG (SEQ ID NO: 207), and a VH CDR3 sequence of AGTQFDY (SEQ ID NO: 208); c) a VH CDR1 sequence of WYPMW (SEQ ID NO: 210), a VH CDR2 sequence of LIEGQGDRTYYADSVKG (SEQ ID NO: 211), and a VH CDR3 sequence of AGDRTAGSRGNSFDY (SEQ ID NO: 212); d) a VH CDR1 sequence of AYPMM (SEQ ID NO: 202), a VH CDR2 sequence of EISPSGSYTYYADSVKG (SEQ ID NO: 215), and a VH CDR3 sequence of DPRKFDY (SEQ ID NO: 216); or e) a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain comprises an amino acid sequence having at least 80%, 85%, 90%, 95%, 96%, 97%, 98%, 99% or 100% identity to any one of SEQ ID NOs: 200, 201, 205, 209, 213, 217, 221, or 280-284. In some embodiments, the VHH comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain comprises an amino acid sequence having at least 80%, 85%, 90%, 95%, 96%, 97%, 98%, 99% or 100% identity to SEQ ID NO:200, 217, or 221. In some embodiments, the second polypeptide chain further comprises an additional binding domain targeting VEGFA. In some embodiments, the additional binding domain targeting VEGFA is a VHH. In some embodiments, both the second binding domain and the additional binding domain targeting VEGFA are both VHHs comprising an identical amino acid sequence, or wherein the VHH positioned C-terminal to the other VHH comprises an additional two proline peptide on its C-terminus as compared to the other VHH, optionally wherein the VHH comprises a two proline peptide on its C-terminus. In some embodiments, the additional binding domain targeting VEGFA comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the additional binding domain targeting VEGFA an amino acid sequence having at least 80%, 85%, 90%, 95%, 96%, 97%, 98%, 99% or 100% identity to SEQ ID NO: 200, 217, or 221. In some embodiments, the second polypeptide chain comprises, in an N-terminal to C-terminal direction, the VH of the Fab, an antibody constant region, an optional peptide linker, the second binding domain, a second optional peptide linker, and the additional binding domain targeting VEGFA. In some embodiments, the second optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence (GGGGS)₂ (SEQ ID NO: 22), (GGGGS)₃ (SEQ ID NO: 29), or (GGGGS)₄ (SEQ ID NO: 30). In some embodiments, the second polypeptide chain comprises the sequence set forth in any one of SEQ ID NOs: 163, 164, 169, or 171.

In some embodiments, the third polypeptide chain comprises, in an N-terminal to C-terminal direction, the IL-2 polypeptide, an optional peptide linker, and an antibody constant region. In some embodiments, the optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the third polypeptide chain comprises, in an N-terminal to C-terminal direction, the IL-2 polypeptide, the optional peptide linker, an antibody constant region, a second optional peptide linker, and the second binding domain or an additional binding domain targeting VEGFA, wherein the additional binding domain targeting VEGFA is present if the second binding is present on the second polypeptide chain or if the second binding domain is also present on the third polypeptide chain. In some embodiments, the second optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the second binding domain or the additional binding domain targeting VEGFA is a VHH. In some embodiments, the VHH comprises a two proline peptide on its C-terminus. In some embodiments, the second binding domain or the additional binding domain targeting VEGFA comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain or the additional binding domain comprises an amino acid sequence having at least 80%, 85%, 90%, 95%, 96%, 97%, 98%, 99% or 100% identity to SEQ ID NO: 200, 217 or 221. In some embodiments, the third polypeptide comprises the second binding domain and the additional binding domain targeting VEGFA separated by an optional peptide linker. In some embodiments, the optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, both the second binding domain and the additional binding domain targeting VEGFA are both VHHs comprising an identical amino acid sequence, or wherein the VHH positioned C-terminal to the other VHH comprises an additional two proline peptide on its C-terminus as compared to the other VHH. In some embodiments, the antibody constant region is an IgG1 or IgG4 constant region, or a portion thereof. In some embodiments, the antibody constant region comprises, in an N-terminal to C-terminal direction, a hinge region, a CH2 domain, and a CH3 domain. In some embodiments, the antibody constant region comprises, in an N-terminal to C-terminal direction, a hinge region portion, a CH2 domain, and a CH3 domain. In some embodiments: a) the hinge region portion comprises an amino acid sequence having at least 80%, 85%, 90%, 95%, 96%, 97%, 98%, 99%, or 100% identity to any one of SEQ ID NOs: 265-271; and/or b) the CH2 and CH3 domains together comprise an amino acid sequence having at least 80%, 85%, 90%, 95%, 96%, 97%, 98%, 99%, or 100% identity to any one of SEQ ID NOs: 229-234. In some embodiments, the third polypeptide chain comprises an amino acid sequence as set forth in any one of SEQ ID NOs: 802, 803, 806, or 807. In some embodiments: the first polypeptide chain comprises the amino acid sequence set forth in SEQ ID NO: 47; the second polypeptide chain comprises the amino acid sequence of any one of SEQ ID NOs: 163, 164, 169, or 171; and the third polypeptide chain comprises the amino acid sequence of any one of SEQ ID NOs: 802, 803, 806, or 807. In some embodiments, the first polypeptide chain, the second polypeptide chain, and the third polypeptide chain comprise, respectively, the amino acid sequences set forth in: SEQ ID NOs: 47, 163, and 802; SEQ ID NOs: 47, 163, and 803; SEQ ID NOs: 47, 164, and 806; SEQ ID NOs: 47, 164, and 807; SEQ ID NOs: 47, 169, and 802; SEQ ID NOs: 47, 169, and 803; SEQ ID NOs: 47, 172, and 802; SEQ ID NOs: 47, 172, and 803; SEQ ID NOs: 47, 163, and 806; SEQ ID NOs: 47, 163, and 807; SEQ ID NOs: 47, 169, and 806; SEQ ID NOs: 47, 169, and 807; SEQ ID NOs: 47, 171, and 809; or SEQ ID NOs: 47, 171, and 810.

In some embodiments, the second binding domain is a Fab having a VH and a VL, wherein the composition comprises a) a first polypeptide chain comprising the VL of the first binding domain; b) a second polypeptide chain comprising the VH of the first binding domain; and c) a third polypeptide chain comprising the IL-2 polypeptide.

In some embodiments, second binding domain Fab comprises a VH having a VH CD1, VH CDR2, and VH CDR3 and a VL having a VL CDR1, CDR2, and CDR3 of any one of the anti-VEGFA antibodies in Table 2A, optionally wherein the VH comprises an amino acid sequence having at least 80%, 85%, 90%, 95%, 96%, 97%, 98%, 99%, or 100% identity to the corresponding VL. In some embodiments, the VH CDR1, VH CDR2, and VH CDR3 are SEQ ID NOs: 123, 124, and 125, respectively and the VL CDR1, VL CDR2, and VL CDR3 are SEQ ID NOs: 128, 129, and 130, respectively. In some embodiments, the VH and VL each have a sequence having at least 80%, 85%, 90%, 95%, 96%, 97%, 98%, 99%, or 100% identity to SEQ ID NOs: 122 and 126, respectively. In some embodiments, the first polypeptide chain comprises, in N- to C-terminal direction, the VL and a light chain constant region. In some embodiments, the light chain constant region comprises an amino acid having at least 80%, 85%, 90%, 95%, 96%, 97%, 98%, 99%, or 100% sequence identity to SEQ ID NO: 276, 277, or 278.

In some embodiments, the second polypeptide chain comprises, in an N-terminal to C-terminal direction, the VH of the Fab of the second binding domain and an antibody constant region. In some embodiments, the antibody constant region is an IgG1 or an IgG4. In some embodiments, the antibody constant region comprises, in an N-terminal to C-terminal direction, a CH1 domain, a hinge region, a CH2 domain, and a CH3 domain. In some embodiments, the CH1 domain comprises an amino acid sequence having at least 80%, 85%, 90%, 95%, 96%, 97%, 98%, 99%, or 100% identity to any one of SEQ ID NOs: 272-275, the hinge region comprises an amino acid sequence having at least 80%, 85%, 90%, 95%, 96%, 97%, 98%, 99%, or 100% identity to any one of SEQ ID NOs 265-271, and/or the CH2 and CH3 domains together comprise an amino acid sequence having at least 80%, 85%, 90%, 95%, 96%, 97%, 98%, 99%, or 100% identity to any one of SEQ ID NOs 229-234. In some embodiments, the second polypeptide chain comprises, in an N-terminal to C-terminal direction, the VH of the Fab, the antibody constant region, an optional peptide linker, and the first binding domain specific for PD-1. In some embodiments, the optional peptide linker is present and comprises a sequence of any one of SEQ ID NOs: 21-30. In some embodiments, the first binding domain is a VHH. In some embodiments, the VHH comprises a VH CDR1, VH CDR2, and VH CDR3 of any one of the anti-PD-1 VHH in Table 1B or Table 1C. In some embodiments, the anti-PD-1 binding domain comprises the CDRS of VHH47, VHH62, VHH70, VHH76, VHH84, or VHH178. In some embodiments, the first binding domain comprises an amino acid sequence having at least 80%, 85%, 90%, 95%, 96%, 97%, 98%, 99% or 100% identity to any one of VHH47, VHH62, VHH70, VHH76, VHH84, or VHH178. In some embodiments, the first binding domain is VHH47. In some embodiments, the second polypeptide chain further comprises an additional binding domain targeting PD-1. In some embodiments, the additional binding domain VHH comprises an identical amino acid sequence compared to the first binding domain. In some embodiments, the first binding domain and the additional binding domain are separated by a peptide linker, optionally wherein the peptide linker comprises a sequence of any one of SEQ ID NOs: 22-30.

In some embodiments, the third polypeptide chain comprises, in an N-terminal to C-terminal direction, a VH of a second Fab specific for VEGFA, an antibody constant region, an optional peptide linker, and the IL-2 polypeptide. In some embodiments, the second Fab is the same as the Fab of the second binding domain. In some embodiments, the antibody constant region of the third polypeptide is an IgG1 or an IgG4. In some embodiments, the antibody constant region of the third polypeptide comprises, in an N-terminal to C-terminal direction, a CH1 domain, a hinge region, a CH2 domain, and a CH3 domain. In some embodiments, the CH1 domain comprises an amino acid sequence having at least 80%, 85%, 90%, 95%, 96%, 97%, 98%, 99%, or 100% identity to any one of SEQ ID NOs: 272-275, the hinge region comprises an amino acid sequence having at least 80%, 85%, 90%, 95%, 96%, 97%, 98%, 99%, or 100% identity to any one of SEQ ID NOs 265-271, and/or the CH2 and CH3 domains together comprise an amino acid sequence having at least 80%, 85%, 90%, 95%, 96%, 97%, 98%, 99%, or 100% identity to any one of SEQ ID NOs 229-234. In some embodiments, the optional peptide linker of the third polypeptide is present and comprises a sequence of any one of SEQ ID NOs: 21-30.

In some embodiments, the IL-2 polypeptide comprises the sequence set forth in SEQ ID NO: 751, 753, 754, or 758.

In some embodiments, the composition comprises only one cytokine.

In an aspect, the present disclosure provides one or more polynucleotides encoding the composition of any one of the embodiments disclosed herein, or a portion thereof.

In an aspect, the present disclosure provides a host cell comprising the composition of any one of the embodiments disclosed herein or the one or more polynucleotides of any of the embodiments disclosed herein.

In an aspect, the present disclosure provides a pharmaceutical composition comprising the composition of any one of the embodiments disclosed herein, and a pharmaceutically acceptable carrier or excipient.

In an aspect, the present disclosure provides a method of treating cancer in a subject in need thereof, the method comprising administering to the subject at therapeutically effective amount of the composition of any one of the embodiments disclosed herein or the pharmaceutical composition of any one of the embodiments disclosed herein.

In an aspect, the present disclosure provides an IL-2 polypeptide comprising a modified B′C′ loop region, wherein the modified B′C′ loop region comprises a deletion of one or more amino acids of the B′C′ loop region between amino acids 73 and 84 of the IL-2 polypeptide, wherein residue position numbering is based on SEQ ID NO: 701 as a reference sequence, and an insertion of a peptide comprising the sequence GDGSIN into the deleted portion of the B′C′ loop region. In some embodiments, the deletion of one or more amino acids of the B′C′ loop region comprises a deletion of 1, 2, 3, 4, 5, 6, 7, 8, 9 or 10 amino acids between amino acids 73 and 84 of the IL-2 polypeptide. In some embodiments, the deletion of one or more amino acids of the B′C′ loop region comprises a deletion of each of amino acids 74-83 of the IL-2 polypeptide. In some embodiments, the inserted peptide consists of the sequence GDGSIN. In some embodiments, the IL-2 polypeptide comprises a peptide sequence having at least 80%, 85%, 90%, 95%, 96%, 97%, 98%, 99%, or 100% sequence identity to amino acids 1-73 of SEQ ID NO: 701 and a peptide sequence having at least 80%, 85%, 90%, 95%, 96%, 97%, 98%, 99%, or 100% sequence identity to amino acids 84-133 of SEQ ID NO: 701. In some embodiments, the IL-2 polypeptide comprises a peptide sequence having at least 80%, 85%, 90%, 95%, 96%, 97%, 98%, 99%, or 100% identity to SEQ ID NO: 783. An IL-2 polypeptide comprising the amino acid substitutions K76A and R81S, wherein residue position numbering is based on SEQ ID NO: 1 as a reference sequence. In some embodiments, the IL-2 polypeptide exhibits reduced binding to heparin compared to the IL-2 polypeptide of SEQ ID NO: 701. In some embodiments, the IL-2 polypeptide exhibits reduced binding to the IL-2 receptor beta subunit compared to the IL-2 of SEQ ID NO: 1. In some embodiments, the IL-2 polypeptide comprises an amino acid substitution at residue N88. In some embodiments, the IL-2 polypeptide comprises an N88D substitution. In some embodiments, the IL-2 polypeptide exhibits reduced binding to the IL-2 receptor gamma subunit. In some embodiments, the IL-2 polypeptide comprises a substitution at any one of residues L12, E15, L19, T123, Q126, or I129. In some embodiments, the IL-2 polypeptide comprises one or more substitutions selected from L12A, L12Y, E15D, E15S, L19A, L19D, T123A, Q126T, or I129A, I129K. In some embodiments, the IL-2 polypeptide comprises any one of the following sets of substitutions: Q126T; I129K; I129A, E15S, T123A; E15D; L12A, L19A, E15S; L12Y, L19D; L12A, L19A; or L19D. In some embodiments, the IL-2 polypeptide comprises an E15D or an L19D substitution. In some embodiments, the IL-2 polypeptide comprises a T3A substitution. In some embodiments, the IL-2 polypeptide comprises a C125S substitution. In some embodiments, the IL-2 polypeptide binds to the IL-2 receptor alpha subunit. In some embodiments, the IL-2 polypeptide comprises the sequence set forth in any one of SEQ ID NOs: 703-774.

In an aspect, the present disclosure provides a fusion polypeptide comprising the IL-2 polypeptide of any one of the embodiments disclosed herein and an additional polypeptide. In some embodiments, the IL-2 polypeptide is fused to the additional polypeptide at its C-terminus. In some embodiments, the additional polypeptide comprises an Fc domain. In some embodiments, the IL-2 polypeptide is connected to the Fc domain via a peptide linker.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIG. 1A shows an exemplary embodiment of a multifunctional immunocytokine as described herein, which comprises an IL-2 polypeptide conjugated to an Fc domain, said Fc domain linked to two Fabs targeting VEGF and two scFvs targeting PD-1.

FIG. 1B shows an analogous multifunctional immunocytokine to that of FIG. 1A, but with an activatable IL-2 polypeptide depicted with its mask intact. The mask is linked to the IL-2 polypeptide by a protease cleavable linker. Upon cleavage of the protease cleavable linker, the mask is able to dissociate, thus allowing the IL-2 polypeptide to bind with the IL-2 receptor and signal.

FIGS. 2A-2D depict formats of Fc domain containing constructs which contain anti-VEGFA and anti-PD-1 binding domains which can be conjugated with a cytokine such as an IL-2 polypeptide to provide an immunocytokine composition according to the instant disclosure. In some embodiments, the anti-VEGFA binding domain comprises the VH and VL of Bevacizumab, or derivatives thereof. In some embodiments, the anti-PD-1 binding domains comprise the VH and VL of LZM-009, Pembrolizumab, or Nivolumab, or derivatives thereof. In some embodiments, such as those depicted in FIG. 1C and FIG. 1D, an Fc domain containing construct comprises a single domain antibody (e.g., a VHH or light chain single domain antibody) as one of the binding domains (e.g., the anti-PD-1 or anti-VEGFA binding domain).

FIGS. 3A-3D depict formats of asymmetric Fc domain containing constructs contain anti-VEGFA and anti-PD-1 binding domains which can be conjugated with a cytokine such as an IL-2 polypeptide to provide an immunocytokine composition according to the instant disclosure. These constructs contain a K248A substitution which facilitate the conjugation of a single cytokine to the construct (e.g., by AJICAP™ technology), thus readily providing an immunocytokine composition which contains only one cytokine attached. In some embodiments, the anti-VEGFA binding domain comprises the VH and VL of Bevacizumab. In some embodiments, the anti-PD-1 binding domains comprise the VH and VL of LZM-009, Pembrolizumab, or Nivolumab. In some embodiments, such as those depicted in FIG. 3C and FIG. 3D, the Fc domain containing construct comprises a single domain antibody (e.g., a VHH or light chain single domain antibody) as one of the binding domains (e.g., the anti-PD-1 or anti-VEGFA binding domain). In some embodiments, the anti-VEGFA single domain antibody binding domain is one of those described herein (e.g., a single domain antibody of any one of SEQ ID NOs: 200, 201, 205, 209, 213, 217, 221, 222, 280-284, or a variant single domain antibody which comprises the CDRs set forth in one of those sequences).

FIGS. 4A-4C depict formats of asymmetric Fc domain containing constructs in bispecific antibody formats containing anti-VEGFA and anti-PD-1 binding domains which can be conjugated with a cytokine such as an IL-2 polypeptide to provide an immunocytokine composition according to the instant disclosure. In the formats depicted, light chain pairing technology must be utilized during manufacture to ensure proper pairing of VH and VL domains in the construct. In some embodiments, the anti-VEGFA binding domain comprises the VH and VL of Bevacizumab, or a derivative thereof. In some embodiments, the anti-PD-1 binding domains comprise the VH and VL of LZM-009, Pembrolizumab, or Nivolumab, or a derivative thereof. In some embodiments, such as that depicted in FIG. 4B, a second anti-VEGFA binding domain is incorporated via fusion to the C-terminus (optionally through a peptide linker) of the Fc domain. The second anti-VEGFA binding domain depicted in FIG. 4B is an scFv. In some embodiments, the second anti-VEGFA binding domain is derived from the same antibody as the first anti-VEGFA binding domain (e.g., the second anti-VEGFA binding domain contains the same VH and VL as the first anti-VEGFA binding domain). A similar embodiment is also depicted in FIG. 4C, which depicts a construct in which the second VEGFA binding domain is a single domain antibody (e.g., a VHH). In some embodiments, the single domain antibody is one of those described herein (e.g., a single domain antibody of any one of SEQ ID NOs: 200, 201, 205, 209, 213, 217, 221, 222, 280-284, or a variant single domain antibody which comprises the CDRs set forth in one of those sequences).

FIGS. 5A and 5B depict formats of asymmetric Fc domain containing constructs in bispecific antibody formats containing anti-VEGFA and anti-PD-1 binding domains which can be conjugated with a cytokine such as an IL-2 polypeptide to provide an immunocytokine composition according to the instant disclosure. This bispecific antibody format utilizes an scFv as one of the binding domains and eliminates the need for light chain pairing technology to be used in their manufacture. In some embodiments, the anti-VEGFA binding domain comprises the VH and VL of Bevacizumab, or derivatives thereof. In some embodiments, the anti-PD-1 binding domains comprise the VH and VL of LZM-009, Pembrolizumab, or Nivolumab, or derivatives thereof.

FIGS. 6A-6C depict formats of immunocytokine compositions according to the instant disclosure in which an IL-2 polypeptide is fused to the Fc domain. In some embodiments, the anti-VEGFA binding domain comprises the VH and VL of Bevacizumab, or derivatives thereof. In some embodiments, the anti-PD-1 binding domains comprise the VH and VL of LZM-009, Pembrolizumab, or Nivolumab, or derivatives thereof.

FIGS. 7A-7I depict formats of immunocytokine compositions according to the instant disclosure which utilize a mask to block activity of the IL-2 polypeptide. Upon cleavage of the cleavable linker, the mask dissociates and renders the IL-2 polypeptide available for signaling with its receptor. In FIGS. 7A-7F, the mask (depicted as an anti-IL-2 scFv) is fused to the IL-2 polypeptide through the cleavable linker. In FIGS. 7G and 711, the mask is fused to the opposite Fc domain as the IL-2 polypeptide. In some embodiments, the anti-VEGFA binding domain comprises the VH and VL of Bevacizumab, or derivatives thereof. In some embodiments, the anti-PD-1 binding domains comprise the VH and VL of LZM-009, Pembrolizumab, or Nivolumab, or derivatives thereof.

FIGS. 8A-8H depict formats of immunocytokine compositions according to the instant disclosure which incorporate single domain antibodies specific for one of VEGFA or PD-1 into the composition.

FIGS. 9A-9H show dynamic light scattering (DLS) profiles of immunocytokine compositions described herein in the presence or absence of VEGF (left panels) and Analytical SEC-HPLC chromatogram of purified compositions with and without VEGF addition (detection: 220 nm) (right panels). FIG. 9A shows results for Composition 116. FIG. 9B shows results for Composition 117. FIG. 9C shows results for Composition 118. FIG. 9D shows results for Composition 119. FIG. 9E shows results for Composition 91. FIG. 9F shows results for Composition 98. FIG. 9G shows results for Composition 94. FIG. 9H shows results for Composition 120.

FIGS. 10A and 10B show the results of IL-2R HEKBlue® reporter assays for various immunocytokine compositions described herein in the presence or absence of VEGF.

FIGS. 11A and 11B show STAT5 induction of parental PD1⁻ NK92 cells and NK92 cells engineered to express human PD-1 by the indicated immunocytokines.

FIGS. 12A and 12B show results of PD-1/PD-L1 blocking assays using immunocytokine composition described herein.

FIGS. 13A and 13B show results of VEGFR blocking assays using immunocytokine compositions described herein.

FIG. 14 shows STAT5 induction of PBMC subpopulations by immunocytokine compositions described herein.

FIG. 15 shows PK results from mice dosed with the indicated immunocytokines.

FIG. 16 shows average tumor growth curves, body weight change, and survival curves in an MC38 bearing C57BI/6 mouse model transgenic for human PD-1 after administration of immunocytokine Composition 116.

FIG. 17 shows average tumor growth curves, body weight change, and survival curves in an MC38 bearing C57BI/6 mouse model transgenic for human PD-1 after administration of immunocytokine Composition 117.

FIG. 18 shows body weight change and average tumor volume in an MKN45 tumor cell model in BalbC/nude mice administered one of Composition 117, Composition 118, or Composition 119.

While certain figures discussed supra include immunocytokine compositions or Fc domain contains scaffolds depicted as having various modifications to the Fc domain, compositions with alternative Fc domains or modifications thereof are also within the scope of the instant disclosure. The depiction of the formats with the indicated Fc modifications is not limiting.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Multifunctional Immunocytokines

Provided herein in an aspect are multifunctional immunocytokines. In some embodiments, the multifunctional immunocytokines comprise a PD-1 binding domain, a VEGFA binding domain, and a cytokine, such as IL-2.

### Binding Domains

An immunocytokine composition described herein comprises one or more binding domains which specifically target one of PD-1 or VEGFA. In some preferred embodiments, an immunocytokine composition comprises both PD-1 and VEGFA binding domains.

A binding domain selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the binding domain is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the binding domain for unrelated amino acid sequences.

In some embodiments, a binding domain of the disclosure can inhibit the action/activity of the substance to which it binds (e.g., by disruption an interaction of PD-1 with its receptor (e.g., PD-L1) and/or by disruption of VEGFA with one or more of its receptors (e.g., VEGFR1, VEGFR2, VEGFR3).

The binding domains of the instant disclosure can be of any desired format which binds specifically to the intended target. As such, the nature of the binding domains is not necessarily limited and can encompass, for example, polypeptides (e.g., antigen binding fragments derived from antibodies or other peptides which bind specifically for the target, such as variants of receptors of the targets or other polypeptides), aptamers (e.g., nucleic acid aptamers), small molecules, and the like. In some embodiments, a binding domain of the instant disclosure is a polypeptide.

In some embodiments, a binding domain of the instant disclosure comprises an antigen binding fragment derived from an antibody, or a variant thereof. Antigen binding fragments of antibodies, including any of the antibodies herein (e.g., the anti-PD-1 antibodies or anti-VEGFA antibodies described below), are contemplated as being used as binding domains in immunocytokine compositions described herein. The terms “antigen binding portion of an antibody,” “antigen binding domain,” “antibody fragment,” or a “functional fragment of an antibody” are used interchangeably herein to refer to one or more fragments of an antibody that retain the ability to specifically bind to an antigen. In some embodiments, such regions refer to the VH and/or the VL of the antibody, or a derivative or portion thereof. Representative antigen binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides).

In some embodiments, a binding domain of the instant disclosure is a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a single domain antibody (e.g., a VHH), a Fab-Fc, a scFv-Fc, or a bispecific antibody. In some embodiments, a binding domain of the instant disclosure is Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a single domain antibody (e.g., a VHH), a Fab-Fc, or a scFv-Fc. In some embodiments, a binding domain of the instant disclosure is Fab, a Fab′, F(ab′)₂, a bispecific F(ab′)₂, a variable fragment (Fv), a single chain variable fragment (scFv), disulfide stabilized Fv (dsFv), a camelid, or a VHH. In some embodiments, a binding domain of the instant disclosure is Fab, a Fab′, F(ab′)₂, a variable fragment (Fv), a single chain variable fragment (scFv), disulfide stabilized Fv (dsFv), a camelid, or a single domain antibody (e.g., a VHH). In some embodiments, a binding domain of the instant disclosure is Fab, a Fab′, a variable fragment (Fv), a single chain variable fragment (scFv), disulfide stabilized Fv (dsFv), or a single domain antibody (e.g., a VHH). In some embodiments, a binding domain of the instant disclosure is Fab, a single chain variable fragment (scFv), or a single domain antibody (e.g., a VHH). In some embodiments, each binding domain of an immunocytokine composition is independently one of those described above. In some embodiments, each binding domain of an immunocytokine composition described herein is independently a Fab, an scFv, or a single domain antibody (e.g., a VHH).

In some embodiments, a binding domain of the instant disclosure comprises a VH derived from an antibody, or a derivative thereof. In some embodiments, the VH retains the CDRs of the VH of the antibody from which the VH of the immunocytokine composition is derived (e.g., a heavy chain first complementary determining region (VH CDR1), a heavy chain second complementary determining region (VH CDR2), and a heavy chain third complementary determining region (VH CDR3)). In some embodiments, the VH retains the CDRS of the VH of the antibody from which it is derived and comprises one or more mutations in the framework region. In some embodiments, the VH retains the CDRs of the VH of the antibody from which it is derived and comprises up to 10, 9, 8, 7, 6, 5, 4, 3, 2, or 1 mutations in the framework region. In some embodiments, each mutation is a conservative mutation. In some embodiments, the binding domain comprises a VH of a corresponding antibody (e.g., with no mutations to the framework region). In some embodiments, the VH is comprises in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. In some embodiments, the VH is comprised in a Fab, a Fab′, an scFv, or a VHH. In some embodiments, the VH is comprised in a Fab, an scFv, or a VHH. In some embodiments, the VH is comprised in a Fab or an scFv. In some embodiments, the VH is a VHH.

In some embodiments, a VHH of the instant disclosure can comprise one or more modifications which improve immunogenicity or reduce binding of pre-existing antibodies to the VHH. Examples of such modifications are described in, for example, U.S. Patent Publication Nos. US20180009888A9 (e.g., extension peptides of 1 to 5 extending beyond the C-terminal “SS” of the VHH, such as those consisting of the amino acids Ala and Gly), US20160207981A1 (e.g., substitutions of the C-terminal “SS” of the VHH, such as with an amino acid or peptide of a sequence E, SE, EG, SEG, EP, EPG, DP, DPG, K, SK, KP, KPG, RP, or RPG and/or substitutions of Leu 11 of the VHH, such as an L11K, L11R, L11D, or L11E substitution), US20140161796A1 (e.g., deletions of certain sequences from the VHH), US20170121399A1 (e.g., substitutions of Leu 11 of the VHH (e.g., L11K or L11V) and/or Leu 89 (e.g., L89T), and Lin et al., “A structure-based engineering approach to abrogate pre-existing antibody binding to biotherapeutics,” PLoS ONE 16(7): e0254944. doi.org/10.1371/journal.pone.0254944 (e.g., the addition of 1, 2, or 3 prolines beyond the C-terminal “SS” of the VHH, such as a two-proline peptide). In some embodiments, a VHH described herein comprises a C-terminal modification of the addition of 1, 2 or 3 prolines to the C-terminus of the VHH (e.g., to the C-terminus of any of the VHHs described herein). In some embodiments, a VHH described herein comprises a C-terminal modification of the addition of 2 prolines to the C-terminus of the VHH. In embodiments where two VHHs are linked in series (e.g., by a flexible peptide linker), in some instance only the C-terminal VHH will comprise the modification (e.g., only the C-terminal VHH comprises the sequence “PP” after the C-terminal “SS” of the VHH). In some instances, both VHHs linked in series will contain the modification (e.g., both will comprise the sequence “PP” after the C-terminal “SS” of each VHH).

In some embodiments, a binding domain of the instant disclosure comprises a VL derived from an antibody, or a derivative thereof. In some embodiments, the VL retains the CDRs of the VL of the antibody from which the VL of the immunocytokine composition is derived (e.g., a light chain first complementary determining region (VL CDR1), a light chain second complementary determining region (VL CDR2), and a light chain third complementary determining region (VL CDR3)). In some embodiments, the VL retains the CDRS of the VL of the antibody from which it is derived and comprises one or more mutations in the framework region. In some embodiments, the VL retains the CDRs of the VL of the antibody from which it is derived and comprises up to 10, 9, 8, 7, 6, 5, 4, 3, 2, or 1 mutations in the framework region. In some embodiments, each mutation is a conservative mutation. In some embodiments, the binding domain comprises a VL of a corresponding antibody (e.g., with no mutations to the framework region). In some embodiments, the VL is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a Fab-Fc, a scFv-Fc, or a bispecific antibody. In some embodiments, the VL is comprised in a Fab, a Fab′, or an scFv. In some embodiments, the VL is comprised in a Fab or an scFv. In some embodiments, the VL is comprised in the same Fab, Fab′, F(ab′)₂, bispecific F(ab′)₂, scFv, bispecific scFv, dsFv, minibody, diabody, bispecific diabody, triabody, tetrabody, maxibody, Fab-Fc, scFv-Fc, or bispecific antibody as the corresponding VH. In some embodiments, the VL is comprised in the same Fab, Fab′, or scFv as the corresponding VH. In some embodiments, the VL is comprised in the same Fab or scFv as the corresponding VH.

In some embodiments, a binding domain of the instant disclosure is a light chain single domain antibody.

In some embodiments, a binding domain specifically binds to one or more epitopes on one or more target antigens. In some embodiments, a binding domain selectively binds to an epitope on a single antigen.

### PD-1 Targeting Binding Domains

In some embodiments, an immunocytokine composition of the instant disclosure comprise one or more binding domains which target programmed cell death protein 1 (PD-1). In some embodiments, a binding domain incorporated into an immunocytokine composition of the disclosure specifically binds to PD-1. In some embodiments, the anti-PD-1 binding domain is capable of disrupting and/or preventing the interaction of PD-1 with programmed cell death ligand 1 (PD-L1).

Programmed cell death protein 1 (also known as PD-1 and CD279), is a cell surface receptor that plays a role in down-regulating the immune system and promoting self-tolerance by suppressing T cell inflammatory activity. PD-1 is an immune cell inhibitory molecule that is expressed on activated B cells, T cells, and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is

(SEQ ID NO: 31)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

In some embodiments, the anti-PD-1 binding domain is comprised in an antigen binding fragment derived from an antibody. For example, the anti-PD-1 binding domain can be derived from any anti-PD-1 antibody known in the art or which can be made according to well understood methods. In some embodiments, the antibody or antigen binding fragment thereof of an immunocytokine composition described herein is derived from an anti-PD-1 antibody or antigen binding fragment.

In one embodiment, an anti-PD-1 binding domain of an immunocytokine composition comprises and antigen binding fragment. In some embodiments, the anti-PD-1 binding domain of the disclosure comprises a combination of a heavy chain variable region (VH) and a light chain variable region (VL) described herein, or of other anti-PD-1 antibodies or antigen binding fragments known in the art. In another embodiment, an anti-PD-1 antibody or an anti-PD-1 antigen binding fragment of the disclosure comprises a combination of complementarity determining regions (VH CDR1, VH CDR2, VH CDR3, VL CDR1, VL CDR2, and VL CDR3) described herein or of other anti-PD-1 antibodies or antigen binding fragments known in the art.

In one embodiment, an anti-PD-1 binding domain of the disclosure comprises the CDRs of an antibody selected from Tislelizumab, Baizean, 0KVO4111B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QVL057INT, H4H7798N, REGN-2810, SAR-439684, Avelumab, BAVENCIO®, 451238, KXG2PJ551I, MSB-0010682, MSB-0010718C, PF-06834635, Durvalumab, IMFINZI®, 28X28X9OKV, MEDI-4736, Lambrolizumab, Pembrolizumab, KEYTRUDA®, MK-3475, SCH-900475, h409AI1, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, P0GVQ9A4S5, TSR-042, WBP-285, Serplulimab, HLX-10, CS-1003, Retifanlimab, 2Y3T5IF01Z, INCMGA-00012, INCMGA-0012, MGA-012, Sasanlimab, LZZ0IC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, 5-95016, Sym-021, LZM-009 (a.k.a., Lipustobart), Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDIO680, or AMP-514 incorporated into a VH and VL. In some embodiments, the anti-PD-1 binding domain comprises the VH and VL of any one of these antibodies. In some embodiments, the anti-PD-1 binding domain comprises the VH and VL of any one of these antibodies or the VH and VL which include the CDRs of these antibodies in one of the following formats: a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a Fab-Fc, a scFv-Fc, or a bispecific antibody. In some embodiments, the VH and VL of any one of these antibodies or the VH and VL which include the CDRs of these antibodies is comprised in a Fab, a Fab′, or an scFv. In some embodiments, the VH and VL any one of these antibodies or of the VH and VL which include the CDRs of these antibodies is comprised in a Fab or an scFv. In some embodiments, the VH and VL of any of these antibodies or the VH and VL which include the CDRs of these antibodies is comprised in a Fab. In some embodiments, the VH and VL of any of these antibodies or the VH and VL which include the CDRs of these antibodies is comprised in an scFv.

In some embodiments, an anti-PD-1 binding domain of the disclosure comprises the CDRs (e.g., VH and VL CDRs) of Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, Pidilizumab, LZM-009 (a.k.a. Lipustobart), or Budigalimab incorporated into a VH and VL. In one embodiment, an anti-PD-1 binding domain of the disclosure comprises the VH and VL of Tislelizumab, Sintilimab, Toripalimab, Terepril, Camrelizumab, Cemiplimab, Pembrolizumab, Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, Pidilizumab, LZM-009 (a.k.a. Lipustobart), or Budigalimab. In some embodiments, the anti-PD-1 binding domain comprises the VH and VL of any one of these antibodies or the VH and VL which include the CDRs of these antibodies in one of the following formats: a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a Fab-Fc, a scFv-Fc, or a bispecific antibody. In some embodiments, the VH and VL of any one of these antibodies or the VH and VL which include the CDRs of these antibodies is comprised in a Fab, a Fab′, or an scFv. In some embodiments, the VH and VL any one of these antibodies or of the VH and VL which include the CDRs of these antibodies is comprised in a Fab or an scFv. In some embodiments, the VH and VL of any of these antibodies or the VH and VL which include the CDRs of these antibodies is comprised in a Fab. In some embodiments, the VH and VL of any of these antibodies or the VH and VL which include the CDRs of these antibodies is comprised in an scFv.

An anti-PD-1 binding domain can comprise a heavy chain, a VH, or a VH-CH1 domain (e.g., as in a Fab) having an amino acid sequence of any one of those set forth in Table 1A, or a portion corresponding to a VH thereof (e.g., the portion depicted in bold). An anti-PD-1 binding domain can comprise (or further comprise) a light chain (e.g., as in a Fab) or VL having an amino acid sequence of any one of those described in Table 1A, or a portion corresponding to a VL thereof. In preferred embodiments, the heavy chain, VH, or VH-CH1 domain and VL or light chain are from the same antibody or antigen binding fragment described in Table 1A.

In some embodiments, the anti-PD-1 binding domain comprises the VH and VL of Nivolumab, Pembrolizumab, LZM-009, Dostarlimab, Sintilimab, Spartalizumab, Tislelizumab, or Cemiplimab. In some embodiment, the anti-PD-1 binding domain comprises the VH and VL of Dostarlimab, Sintilimab, Spartalizumab, or Tislelizumab. In some embodiments, the anti-PD-1 binding domain comprises the VH and VL of Nivolumab, Pembrolizumab, LZM-009 (a.k.a. Lipustobart), or Cemiplimab. In some embodiments, the anti-PD-1 binding domain comprises the VH and VL of Nivolumab. In some embodiments, the anti-PD-1 binding domain comprises the VH and VL of Nivolumab in a Fab or scFv format. In some embodiments, the anti-PD-1 binding domain comprises the VH and VL of LZM-009 (a.k.a. Lipustobart). In some embodiments, the anti-PD-1 binding domain comprises the VH and VL of LZM-009 (a.k.a. Lipustobart) in a Fab or scFv format.

TABLES 1A and 1B provide the sequences of exemplary anti-PD-1 antibodies and anti-PD-1 antigen binding fragments which contain sets of CDRs which can be incorporated into VHs and/or VLs and used as anti-PD-1 binding domains as described herein. In some embodiments, the VHs and VLs of the antibodies in Table 1A are incorporated into anti-PD-1 binding domains (e.g., in a Fab or scFv format). In some embodiments, CDRs of a VH described in Table 1A or 1B are incorporated into a binding domain as a VHH. In some embodiments, a VH as described in Table 1A or 1B is incorporated into a binding domain as a VHH.

In some instances, the SEQ ID NOs listed in Table 1A contain full-length heavy or light chains of the indicated antibodies with the VH or VL respectively indicated in bold. Where there is a reference herein to a VH or VL of a SEQ ID NO in Table 1A which contains a full-length heavy or light chain, it is intended to reference the bolded portion of the sequence. For example, reference to “a VH having an amino acid sequence shown in SEQ ID NO: 32” refers to the bolded portion of SEQ ID NO: 32 in Table 1A.

An anti-PD-1 binding domain can comprise a VH having an amino acid sequence of any one of SEQ ID NOS: 32, 34, 36, 38, 40, 46, 48, 50, 52, 54, 56, 58, 60, 62, 64, 66, 68, 70, 372, 74, 76, and 78. An anti-PD-1 binding domain can comprise (or further comprise) a VL having an amino acid sequence of any one of SEQ ID NOS: 33, 35, 37, 39, 41, 47, 49, 51, 53, 55, 57, 59, 61, 63, 65, 67, 69, 71, 73, 75, 77, and 79.

An anti-PD-1 binding domain can comprise a heavy chain or VH having an amino acid sequence of any one of SEQ ID NOS: 32, 34, 36, 38, 40, 46, 48, 50, 52, 54, 56, 58, 60, 62, 64, 66, 68, 70, 72, 74, 76, and 78, or a portion corresponding to a VH thereof (e.g., the portion depicted in bold). An anti-PD-1 binding domain can comprise (or further comprise) a light chain or VL having an amino acid sequence of any one of SEQ ID NOS: 33, 35, 37, 39, 41, 47, 49, 51, 53, 55, 57, 59, 61, 63, 65, 67, 69, 71, 73, 75, 77, and 79, or a portion corresponding to a VL thereof.

In one instance, an anti-PD-1 binding domain comprises a VH having an amino acid sequence shown in SEQ ID NO: 32, and a VL having an amino acid sequence shown in SEQ ID NO: 33. In another instance, an anti-PD-1 binding domain comprises a VH having an amino acid sequence shown in SEQ ID NO: 34, and a VL having an amino acid sequence shown in SEQ ID NO: 35. In another instance, an anti-PD-1 binding domain comprises a VH having an amino acid sequence shown in SEQ ID NO:36, and a VL having an amino acid sequence shown in SEQ ID NO: 37. In another instance, an anti-PD-1 binding domain comprises a VH having an amino acid sequence shown in SEQ ID NO: 38, and a VL having an amino acid sequence shown in SEQ ID NO: 39. In another instance, an anti-PD-1 binding domain comprises a VH having an amino acid sequence shown in SEQ ID NO: 40, and a VL having an amino acid sequence shown in SEQ ID NO: 41. In another instance, an anti-PD-1 binding domain comprises a VH having an amino acid sequence shown in SEQ ID NO: 46, and a VL having an amino acid sequence shown in SEQ ID NO: 47. In another instance, an anti-PD-1 binding domain comprises a VH having an amino acid sequence shown in SEQ ID NO: 48, and a VL having an amino acid sequence shown in SEQ ID NO: 49. In another instance, an anti-PD-1 binding domain comprises a VH having an amino acid sequence shown in SEQ ID NO: 50, and a VL having an amino acid sequence shown in SEQ ID NO: 51. In another instance, an anti-PD-1 binding domain comprises a VH having an amino acid sequence shown in SEQ ID NO: 52, and a VL having an amino acid sequence shown in SEQ ID NO: 53. In another instance, an anti-PD-1 binding domain comprises a VH having an amino acid sequence shown in SEQ ID NO: 54, and a VL having an amino acid sequence shown in SEQ ID NO: 55. In another instance, an anti-PD-1 binding domain comprises a VH having an amino acid sequence shown in SEQ ID NO: 56, and a VL having an amino acid sequence shown in SEQ ID NO: 57. In another instance, an anti-PD-1 binding domain comprises a VH having an amino acid sequence shown in SEQ ID NO: 58, and a VL having an amino acid sequence shown in SEQ ID NO: 59. In another instance, an anti-PD-1 binding domain comprises a VH having an amino acid sequence shown in SEQ ID NO: 60, and a VL having an amino acid sequence shown in SEQ ID NO: 61. In another instance, an anti-PD-1 binding domain comprises a VH having an amino acid sequence shown in SEQ ID NO: 62, and a VL having an amino acid sequence shown in SEQ ID NO: 63. In another instance, an anti-PD-1 binding domain comprises a VH having an amino acid sequence shown in SEQ ID NO: 64, and a VL having an amino acid sequence shown in SEQ ID NO: 65. In another instance, an anti-PD-1 binding domain comprises a VH having an amino acid sequence shown in SEQ ID NO: 66, and a VL having an amino acid sequence shown in SEQ ID NO: 67. In another instance, an anti-PD-1 binding domain comprises a VH having an amino acid sequence shown in SEQ ID NO: 68, and a VL having an amino acid sequence shown in SEQ ID NO: 69. In another instance, an anti-PD-1 binding domain comprises a VH having an amino acid sequence shown in SEQ ID NO: 70, and a VL having an amino acid sequence shown in SEQ ID NO: 71. In another instance, an anti-PD-1 binding domain comprises a VH having an amino acid sequence shown in SEQ ID NO: 72, and a VL having an amino acid sequence shown in SEQ ID NO: 73. In another instance, an anti-PD-1 binding domain comprises a VH having an amino acid sequence shown in SEQ ID NO: 74, and a VL having an amino acid sequence shown in SEQ ID NO: 75. In another instance, an anti-PD-1 binding domain comprises a VH having an amino acid sequence shown in SEQ ID NO: 76, and a VL having an amino acid sequence shown in SEQ ID NO: 77. In another instance, an anti-PD-1 binding domain comprises a VH having an amino acid sequence shown in SEQ ID NO: 78, and a VL having an amino acid sequence shown in SEQ ID NO: 79. In another instance, an anti-PD-1 binding domain comprises a VH having an amino acid sequence shown in SEQ ID NO: 9, and a VL having an amino acid sequence shown in SEQ ID NO: 10. In another instance, an anti-PD-1 binding domain comprises a VH having an amino acid sequence shown in SEQ ID NO: 11, and a VL having an amino acid sequence shown in SEQ ID NO: 12.

In one instance, an anti-PD-1 binding domain comprises a VH CDR1 having an amino acid sequence of SEQ ID NO: 80, a VH CDR2 having an amino acid sequence of SEQ ID NO: 81, a VH CDR3 having an amino acid sequence of SEQ ID NO: 82, VL CDR1 having an amino acid sequence of SEQ ID NO: 83, a VL CDR2 having an amino acid sequence of SEQ ID NO: 84, and a VL CDR3 having an amino acid sequence of SEQ ID NO: 85. In some embodiments, the anti-PD-1 binding domain comprises a corresponding VH and VL having at least 80%, 85%, 90%, 95%, 96%, 97%, 98%, 99%, or 100% identity to the VH and VL of the parent antibody (e.g., contains the indicated CDRs and the VH and VL each comprise the indicated sequence identity overall).

In one instance, an anti-PD-1 binding domain comprises a VH CDR1 having an amino acid sequence of SEQ ID NO: 86, a VH CDR2 having an amino acid sequence of SEQ ID NO: 87, a VH CDR3 having an amino acid sequence of SEQ ID NO: 88, VL CDR1 having an amino acid sequence of SEQ ID NO: 89, a VL CDR2 having an amino acid sequence of SEQ ID NO: 90, and a VL CDR3 having an amino acid sequence of SEQ ID NO: 91. In some embodiments, the anti-PD-1 binding domain comprises a corresponding VH and VL having at least 80%, 85%, 90%, 95%, 96%, 97%, 98%, 99%, or 100% identity to the VH and VL of the parent antibody (e.g., contains the indicated CDRs and the VH and VL each comprise the indicated sequence identity overall).

In one instance, an anti-PD-1 binding domain comprises a VH CDR1 having an amino acid sequence of SEQ ID NO: 92, a VH CDR2 having an amino acid sequence of SEQ ID NO: 93, a VH CDR3 having an amino acid sequence of SEQ ID NO: 94, VL CDR1 having an amino acid sequence of SEQ ID NO: 95, a VL CDR2 having an amino acid sequence of SEQ ID NO: 96, and a VL CDR3 having an amino acid sequence of SEQ ID NO: 97. In some embodiments, the anti-PD-1 binding domain comprises a corresponding VH and VL having at least 80%, 85%, 90%, 95%, 96%, 97%, 98%, 99%, or 100% identity to the VH and VL of the parent antibody (e.g., contains the indicated CDRs and the VH and VL each comprise the indicated sequence identity overall).

In one instance, an anti-PD-1 binding domain comprises a VH CDR1 having an amino acid sequence of SEQ ID NO: 98, a VH CDR2 having an amino acid sequence of SEQ ID NO: 99, a VH CDR3 having an amino acid sequence of SEQ ID NO: 100, VL CDR1 having an amino acid sequence of SEQ ID NO: 89, a VL CDR2 having an amino acid sequence of SEQ ID NO: 102, and a VL CDR3 having an amino acid sequence of SEQ ID NO: 103. In some embodiments, the anti-PD-1 binding domain comprises a corresponding VH and VL having at least 80%, 85%, 90%, 95%, 96%, 97%, 98%, 99%, or 100% identity to the VH and VL of the parent antibody (e.g., contains the indicated CDRs and the VH and VL each comprise the indicated sequence identity overall).

In one instance, an anti-PD-1 binding domain comprises a VH CDR1 having an amino acid sequence of SEQ ID NO: 113, a VH CDR2 having an amino acid sequence of SEQ ID NO: 114, a VH CDR3 having an amino acid sequence of SEQ ID NO: 115, VL CDR1 having an amino acid sequence of SEQ ID NO: 83, a VL CDR2 having an amino acid sequence of SEQ ID NO: 117, and a VL CDR3 having an amino acid sequence of SEQ ID NO: 118. In some embodiments, the anti-PD-1 binding domain comprises a corresponding VH and VL having at least 80%, 85%, 90%, 95%, 96%, 97%, 98%, 99%, or 100% identity to the VH and VL of the parent antibody (i.e., SEQ ID NOs: 76 and 77, respectively) (e.g., contains the indicated CDRs and the VH and VL each comprise the indicated sequence identity overall).

In some embodiments, an immunocytokine composition comprises one of the antibodies described in Table 1A or 1B below (optionally comprising one or more modifications to the Fc domain, hinge region, or other modification described herein) fused to a binding domain specific for VEGFA (e.g., any of the VEGFA binding domains described herein). In some embodiments, the binding domain specific for VEGFA is fused to the C-terminus of the heavy chain of the antibody described in Table 1A or 1B. In some embodiments, the binding domain specific for VEGFA fused to the antibody of Table 1A or 1B is an anti-VEGFA single-domain antibody as described herein.

In some embodiments, an anti-PD-1 binding domain comprises a single domain antibody. In some embodiments, the anti-PD-1 binding domain of a dual binding composition is a single domain antibody described in Table 1A or 1, or a variant thereof.

In some embodiments, the anti-PD-1 binding domain is a single domain antibody comprising a CDR1 as set forth in SEQ ID NO: 2, a CDR2 as set forth in SEQ ID NO: 3, and a CDR3 as set forth in SEQ ID NO: 4. In some embodiments, the CDRs are comprised in a VHH. In some embodiments, the anti-PD-1 binding domain comprises an amino acid sequence having 80%, 85%, 90%, 95%, 96%, 97%, 98%, 99%, or 100% to the sequence set forth in SEQ ID NO: 1 (e.g., the anti-PD-1 binding domain comprises the indicated sequence identity to SEQ ID NO: 1 and retains the CDRs).

In some embodiments, the anti-PD-1 binding domain is a single domain antibody comprising a CDR1 as set forth in SEQ ID NO: 6, a CDR2 as set forth in SEQ ID NO: 7, and a CDR3 as set forth in SEQ ID NO: 8. In some embodiments, the CDRs are comprised in a VHH. In some embodiments, the anti-PD-1 binding domain comprises an amino acid sequence having 80%, 85%, 90%, 95%, 96%, 97%, 98%, 99%, or 100% to the sequence set forth in SEQ ID NO: 5 (e.g., the anti-PD-1 binding domain comprises the indicated sequence identity to SEQ ID NO: 5 and retains the CDRs).

In some embodiments, the anti-PD-1 binding domain is a single domain antibody comprising a CDR1 as set forth in SEQ ID NO: 14, a CDR2 as set forth in SEQ ID NO: 15, and a CDR3 as set forth in SEQ ID NO: 16. In some embodiments, the CDRs are comprised in a VHH. In some embodiments, the anti-PD-1 binding domain comprises an amino acid sequence having 80%, 85%, 90%, 95%, 96%, 97%, 98%, 99%, or 100% to the sequence set forth in SEQ ID NO: 13 (e.g., the anti-PD-1 binding domain comprises the indicated sequence identity to SEQ ID NO: 13 and retains the CDRs).

In some embodiments, the anti-PD-1 binding domain is a single domain antibody comprising a CDR1 as set forth in SEQ ID NO: 18, a CDR2 as set forth in SEQ ID NO: 19, and a CDR3 as set forth in SEQ ID NO: 20. In some embodiments, the CDRs are comprised in a VHH. In some embodiments, the anti-PD-1 binding domain comprises an amino acid sequence having 80%, 85%, 90%, 95%, 96%, 97%, 98%, 99%, or 100% to the sequence set forth in SEQ ID NO: 17 (e.g., the anti-PD-1 binding domain comprises the indicated sequence identity to SEQ ID NO: 17 and retains the CDRs).

In some embodiments, the anti-PD-1 binding domain is a single domain antibody comprising a CDR1 as set forth in SEQ ID NO: 288, a CDR2 as set forth in SEQ ID NO: 289, and a CDR3 as set forth in SEQ ID NO: 290. In some embodiments, the CDRs are comprised in a VHH. In some embodiments, the anti-PD-1 binding domain comprises an amino acid sequence having 80%, 85%, 90%, 95%, 96%, 97%, 98%, 99%, or 100% to the sequence set forth in SEQ ID NO: 287 (e.g., the anti-PD-1 binding domain comprises the indicated sequence identity to SEQ ID NO: 287 and retains the CDRs).

In some embodiments, an immunocytokine composition comprises one or two anti-PD-binding domains. In some embodiments, the immunocytokine composition comprises one anti-PD-1 binding domain. In some embodiments, the immunocytokine composition comprises two anti-PD-1 binding domains. In some embodiments, each anti-PD-1 binding domain is a Fab or scFv. In some embodiments, each anti-PD-1 binding domain is a single domain antibody. In some embodiments, the immunocytokine composition comprises two copies of the same anti-PD-1 binding domain. In some embodiments, the immunocytokine composition comprises two anti-PD-1 Fabs. In some embodiments, the immunocytokine composition comprises one anti-PD-1 Fab and one anti-Pd-1 scFv. In some embodiments, the anti-PD-1 Fab and the anti-PD-1 scFv comprise the same VH and VL. In some embodiments, the immunocytokine composition comprises one anti-PD-1 Fab and one anti-PD-1 single domain antibody.

TABLE 1A
Exemplary Antibodies Targeting PD-1 From Which anti-PD-1 binding
domains can be derived

| Antibody | | | |
|---|---|---|---|
| or Ag- | | | |
| binding | Antigen | | SEQ |
| fragment | Bound | Sequence | ID NO |
| Tislelizumab, | PD-1 | QVQLQESGPGLVKPSETLSLTCTVSGFSLTSYGVHWIRQPPGK | 32 |
| Baizean, | | GLEWIGVIYADGSTNYNPSLKSRVTISKDTSKNQVSLKLSSVT | |
| 0KVO411B3N, | | AADTAVYYCARAYGNYWYIDVWGQGTTVTVSSASTKGPSVFP | |
| BGB-A317, | | LAPCSRSTSESTAALGCLVKDYFPEPVTVSWNSGALTSGVHTFPA | |
| hu317- | | VLQSSGLYSLSSVVTVPSSSLGTKTYTCNVDHKPSNTKVDKRVES | |
| 1/IgG4mt2 | | KYGPPCPPCPAPPVAGGPSVFLFPPKPKDTLMISRTPEVTCVVVAV | |
| Heavy | | SQEDPEVQFNWYVDGVEVHNAKTKPREEQFNSTYRVVSVLTVV | |
| Chain (VH | | HQDWLNGKEYKCKVSNKGLPSSIEKTISKAKGQPREPQVYTLPPS | |
| in Bold) | | QEEMTKNQVSLTCLVKGFYPSDIAVEWESNGQPENNYKTTPPVL | |
| | | DSDGSFFLYSKLTVDKSRWQEGNVFSCSVMHEALHNHYTQKSLS | |
| | | LSLGK | |
| Tislelizumab, | PD-1 | DIVMTQSPDSLAVSLGERATINCKSSESVSNDVAWYQQKPGQP | 33 |
| Baizean, | | PKLLINYAFHRFTGVPDRFSGSGYGTDFTLTISSLQAEDVAVY | |
| 0KVO411B3N, | | YCHQAYSSPYTFGQGTKLEIKRTVAAPSVFIFPPSDEQLKSGTAS | |
| BGB-A317, | | VVCLLNNFYPREAKVQWKVDNALQSGNSQESVTEQDSKDSTYS | |
| hu317- | | LSSTLTLSKADYEKHKVYACEVTHQGLSSPVTKSFNRGEC | |
| 1/IgG4mt2 | | | |
| Light Chain | | | |
| (VL in | | | |
| Bold) | | | |
| Sintilimab, | PD-1 | QVQLVQSGAEVKKPGSSVKVSCKASGGTFSSYAISWVRQAPG | 34 |
| Tyvyt, IBI- | | QGLEWMGLIIPMEDTAGYAQKFQGRVAITVDESTSTAYMELS | |
| 308 Heavy | | SLRSEDTAVYYCARAEHSSTGTFDYWGQGTLVTVSSASTKGPS | |
| Chain (VH | | VFPLAPCSRSTSESTAALGCLVKDYFPEPVTVSWNSGALTSGVHT | |
| in Bold) | | FPAVLQSSGLYSLSSVVTVPSSSLGTKTYTCNVDHKPSNTKVDKR | |
| | | VESKYGPPCPPCPAPEFLGGPSVFLFPPKPKDTLMISRTPEVTCVV | |
| | | VDVSQEDPEVQFNWYVDGVEVHNAKTKPREEQFNSTYRVVSVL | |
| | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLGK | |
| Sintilimab, | PD-1 | DIQMTQSPSSVSASVGDRVTITCRASQGISSWLAWYQQKPGK | 35 |
| Tyvyt, IBI- | | APKLLISAASSLQSGVPSRFSGSGSGTDFTLTISSLQPEDFATYY | |
| 308 Light | | CQQANHLPFTFGGGTKVEIKRTVAAPSVFIFPPSDEQLKSGTAS | |
| Chain (VL | | VVCLLNNFYPREAKVQWKVDNALQSGNSQESVTEQDSKDSTYS | |
| in Bold) | | LSSTLTLSKADYEKHKVYACEVTHQGLSSPVTKSFNRGEC | |
| Toripalimab, | PD-1 | QGQLVQSGAEVKKPGASVKVSCKASGYTFTDYEMHWVRQA | 36 |
| TeRuiPuLi, | | PIHGLEWIGVIESETGGTAYNQKFKGRVTITADKSTSTAYMEL | |
| Terepril, | | SSLRSEDTAVYYCAREGITTVATTYYWYFDVWGQGTTVTVSS | |
| Tuoyi, JS- | | ASTKGPSVFPLAPCSRSTSESTAALGCLVKDYFPEPVTVSWNSGA | |
| 001, TAB- | | LTSGVHTFPAVLQSSGLYSLSSVVTVPSSSLGTKTYTCNVDHKPS | |
| 001 Heavy | | NTKVDKRVESKYGPPCPPCPAPEFLGGPSVFLFPPKPKDTLMISRT | |
| Chain (VH | | PEVTCVVVDVSQEDPEVQFNWYVDGVEVHNAKTKPREEQFNST | |
| in Bold) | | YRVVSVLTVLHQDWLNGKEYKCKVSNKGLPSSIEKTISKAKGQP | |
| | | REPQVYTLPPSQEEMTKNQVSLTCLVKGFYPSDIAVEWESNGQPE | |
| | | NNYKTTPPVLDSDGSFFLYSRLTVDKSRWQEGNVFSCSVMHEAL | |
| | | HNHYTQKSLSLSLGK | |
| Toripalimab, | PD-1 | DVVMTQSPLSLPVTLGQPASISCRSSQSIVHSNGNTYLEWYLQ | 37 |
| TeRuiPuLi, | | KPGQSPQLLIYKVSNRFSGVPDRFSGSGSGTDFTLKISRVEAED | |
| Terepril, | | VGVYYCFQGSHVPLTFGQGTKLEIKRTVAAPSVFIFPPSDEQLK | |
| Tuoyi, JS- | | SGTASVVCLLNNFYPREAKVQWKVDNALQSGNSQESVTEQDSK | |
| 001, TAB- | | DSTYSLSSTLTLSKADYEKHKVYACEVTHQGLSSPVTKSFNRGEC | |
| 001 Light | | | |
| Chain (VL | | | |
| in Bold) | | | |
| Camrelizumab, | PD-1 | EVQLVESGGGLVQPGGSLRLSCAASGFTFSSYMMSWVRQAP | 38 |
| HR- | | GKGLEWVATISGGGANTYYPDSVKGRFTISRDNAKNSLYLQM | |
| 301210, | | NSLRAEDTAVYYCARQLYYFDYWGQGTTVTVSSASTKGPSVF | |
| INCSHR- | | PLAPCSRSTSESTAALGCLVKDYFPEPVTVSWNSGALTSGVHTFP | |
| 01210, | | AVLQSSGLYSLSSVVTVPSSSLGTKTYTCNVDHKPSNTKVDKRVE | |
| SHR-1210 | | SKYGPPCPPCPAPEFLGGPSVFLFPPKPKDTLMISRTPEVTCVVVD | |
| Heavy | | VSQEDPEVQFNWYVDGVEVHNAKTKPREEQFNSTYRVVSVLTV | |
| Chain (VH | | LHQDWLNGKEYKCKVSNKGLPSSIEKTISKAKGQPREPQVYTLPP | |
| in Bold) | | SQEEMTKNQVSLTCLVKGFYPSDIAVEWESNGQPENNYKTTPPV | |
| | | LDSDGSFFLYSRLTVDKSRWQEGNVFSCSVMHEALHNHYTQKSL | |
| | | SLSLGK | |
| Camrelizumab, | PD-1 | DIQMTQSPSSLSASVGDRVTITCLASQTIGTWLTWYQQKPGK | 39 |
| HR- | | APKLLIYTATSLADGVPSRFSGSGSGTDFTLTISSLQPEDFATY | |
| 301210, | | YCQQVYSIPWTFGGGTKVEIKRTVAAPSVFIFPPSDEQLKSGTA | |
| INCSHR- | | SVVCLLNNFYPREAKVQWKVDNALQSGNSQESVTEQDSKDSTYS | |
| 01210, | | LSSTLTLSKADYEKHKVYACEVTHQGLSSPVTKSFNRGEC | |
| SHR-1210 | | | |
| Light Chain | | | |
| (Light | | | |
| Chain in | | | |
| Bold) | | | |
| Cemiplimab, | PD-1 | EVQLLESGGVLVQPGGSLRLSCAASGFTFSNFGMTWVRQAPG | 40 |
| Cemiplimab- | | KGLEWVSGISGGGRDTYFADSVKGRFTISRDNSKNTLYLQMN | |
| rwlc, | | SLKGEDTAVYYCVKWGNIYFDYWGQGTLVTVSSASTKGPSVF | |
| LIBTAYO ®, | | PLAPCSRSTSESTAALGCLVKDYFPEPVTVSWNSGALTSGVHTFP | |
| 6QVL057INT, | | AVLQSSGLYSLSSVVTVPSSSLGTKTYTCNVDHKPSNTKVDKRVE | |
| H4H7798N, | | SKYGPPCPPCPAPEFLGGPSVFLFPPKPKDTLMISRTPEVTCVVVD | |
| REGN- | | VSQEDPEVQFNWYVDGVEVHNAKTKPREEQFNSTYRVVSVLTV | |
| 2810, SAR- | | LHQDWLNGKEYKCKVSNKGLPSSIEKTISKAKGQPREPQVYTLPP | |
| 439684 | | SQEEMTKNQVSLTCLVKGFYPSDIAVEWESNGQPENNYKTTPPV | |
| Heavy | | LDSDGSFFLYSRLTVDKSRWQEGNVFSCSVMHEALHNHYTQKSL | |
| Chain (VH | | SLSLGK | |
| in Bold) | | | |
| Cemiplimab, | PD-1 | DIQMTQSPSSLSASVGDSITITCRASLSINTFLNWYQQKPGKAP | 41 |
| Cemiplimab- | | NLLIYAASSLHGGVPSRFSGSGSGTDFTLTIRTLQPEDFATYYC | |
| rwlc, | | QQSSNTPFTFGPGTVVDFRRTVAAPSVFIFPPSDEQLKSGTASVV | |
| LIBTAYO ®, | | CLLNNFYPREAKVQWKVDNALQSGNSQESVTEQDSKDSTYSLSS | |
| 6QVL057INT, | | TLTLSKADYEKHKVYACEVTHQGLSSPVTKSFNRGEC | |
| H4H7798N, | | | |
| REGN- | | | |
| 2810, SAR- | | | |
| 439684 | | | |
| Light Chain | | | |
| (VL in | | | |
| Bold) | | | |
| Lambrolizumab, | PD-1 | QVQLVQSGVEVKKPGASVKVSCKASGYTFTNYYMYWVRQAP | 46 |
| Pembrolizumab, | | GQGLEWMGGINPSNGGTNFNEKFKNRVTLTTDSSTTTAYME | |
| KEYTRUDA ®, | | LKSLQFDDTAVYYCARRDYRFDMGFDYWGQGTTVTVSSAST | |
| MK- | | KGPSVFPLAPCSRSTSESTAALGCLVKDYFPEPVTVSWNSGALTS | |
| 3475, SCH- | | GVHTFPAVLQSSGLYSLSSVVTVPSSSLGTKTYTCNVDHKPSNTK | |
| 900475, | | VDKRVESKYGPPCPPCPAPEFLGGPSVFLFPPKPKDTLMISRTPEV | |
| h409A11 | | TCVVVDVSQEDPEVQFNWYVDGVEVHNAKTKPREEQFNSTYRV | |
| Heavy | | VSVLTVLHQDWLNGKEYKCKVSNKGLPSSIEKTISKAKGQPREPQ | |
| Chain (VH | | VYTLPPSQEEMTKNQVSLTCLVKGFYPSDIAVEWESNGQPENNY | |
| in Bold) | | KTTPPVLDSDGSFFLYSRLTVDKSRWQEGNVFSCSVMHEALHNH | |
| | | YTQKSLSLSLGK | |
| | | VH CDR1: NYYMY (SEQ ID NO: 80) | |
| | | VH CDR2: GINPSNGGTNFNEKFKN (SEQ ID NO: 81) | |
| | | VH CDR3: RDYRFDMGFDY (SEQ ID NO: 82) | |
| Lambrolizumab, | PD-1 | EIVLTQSPATLSLSPGERATLSCRASKGVSTSGYSYLHWYQQK | 47 |
| Pembrolizumab, | | PGQAPRLLIYLASYLESGVPARFSGSGSGTDFTLTISSLEPEDF | |
| KEYTRUDA ®, | | AVYYCQHSRDLPLTFGGGTKVEIKRTVAAPSVFIFPPSDEQLKS | |
| MK- | | GTASVVCLLNNFYPREAKVQWKVDNALQSGNSQESVTEQDSKD | |
| 3475, SCH- | | STYSLSSTLTLSKADYEKHKVYACEVTHQGLSSPVTKSFNRGEC | |
| 900475, | | VL CDR1: RASKGVSTSGYSYLH (SEQ ID NO: 83) | |
| h409A11 | | VL CDR2: LASYLES (SEQ ID NO: 84) | |
| Light | | VL CDR3: QHSRDLPLT (SEQ ID NO: 85) | |
| Chain (VL | | | |
| in Bold) | | | |
| Lambrolizumab, | PD-1 | QVQLVQSGVEVKKPGASVKVSCKASGYTFTNYYMYWVRQAPG | 48 |
| Pembrolizumab, | | QGLEWMGGFPSNGGTNFNEKFKNRVTLTTDSSTTTAYMELKSLQ | |
| KEYTRUDA ®, | | FDDTAVYYCARRDYRFDMGFDYWGQGTTVTVSS | |
| MK- | | VH CDR1: NYYMY (SEQ ID NO: 80) | |
| 3475, SCH- | | VH CDR2: GINPSNGGTNFNEKFKN (SEQ ID NO: 81) | |
| 900475, | | VH CDR3: RDYRFDMGFDY (SEQ ID NO: 82) | |
| h409A11 | | | |
| VH | | | |
| Lambrolizumab, | PD-1 | EIVLTQSPATLSLSPGERATLSCRASKGVSTSGYSYLHWYQQKPG | 49 |
| Pembrolizumab, | | QAPRLLIYLASYLESGVPARFSGSGSGTDFTLTISSLEPEDFAVYYC | |
| KEYTRUDA ®, | | QHSRDLPLTFGGGTKVEIK | |
| MK- | | VL CDR1: RASKGVSTSGYSYLH (SEQ ID NO: 83) | |
| 3475, SCH- | | VL CDR2: LASYLES (SEQ ID NO: 84) | |
| 900475, | | VL CDR3: QHSRDLPLT (SEQ ID NO: 85) | |
| h409A11 | | | |
| VL | | | |
| Nivolumab, | PD-1 | QVQLVESGGGVVQPGRSLRLDCKASGITFSNSGMHWVRQAPGK | 50 |
| Nivolumab | | GLEWVAVIWYDGSKRYYADSVKGRFTISRDNSKNTLFLQMNSLR | |
| BMS, | | AEDTAVYYCATNDDYWGQGTLVTVSS | |
| OPDIVO ®, | | VH CDR1: NSGMH (SEQ ID NO: 86) | |
| BMS- | | VH CDR2: VIWYDGSKRYYADSVKG (SEQ ID NO: 87) | |
| 936558, | | VH CDR3: NDDY (SEQ ID NO: 88) | |
| MDX-1106, | | | |
| ONO-4538 | | | |
| VH | | | |
| Nivolumab, | PD-1 | EIVLTQSPATLSLSPGERATLSCRASQSVSSYLAWYQQKPGQAPR | 51 |
| Nivolumab | | LLIYDASNRATGIPARFSGSGSGTDFTLTISSLEPEDFAVYYCQQSS | |
| BMS, | | NWPRTFGQGTKVEIK | |
| OPDIVO ®, | | VL CDR1: RASQSVSSYLA (SEQ ID NO: 89) | |
| BMS- | | VL CDR2: DASNRAT (SEQ ID NO: 90) | |
| 936558, | | VL CDR3: QQSSNWPRT (SEQ ID NO: 91) | |
| MDX-1106, | | | |
| ONO-4538 | | | |
| VL | | | |
| Prolgolimab, | PD-1 | QVQLVQSGGGLVQPGGSLRLSCAASGFTFSSYWMYWVRQVP | 52 |
| Forteca, | | GKGLEWVSAIDTGGGRTYYADSVKGRFAISRVNAKNTMYLQ | |
| BCD-100 | | MNSLRAEDTAVYYCARDEGGGTGWGVLKDWPYGLDAWGQ | |
| Heavy | | GTLVTVSSASTKGPSVFPLAPSSKSTSGGTAALGCLVKDYFPEPV | |
| Chain (VH | | TVSWNSGALTSGVHTFPAVLQSSGLYSLSSVVTVPSSSLGTQTYIC | |
| in Bold) | | NVNHKPSNTKVDKRVEPKSCDKTHTCPPCPAPEAAGGPSVFLFPP | |
| | | KPKDTLMISRTPEVTCVVVDVSHEDPEVKFNWYVDGVEVHNAK | |
| | | TKPREEQYNSTYRVVSVLTVLHQDWLNGKEYKCKVSNKALPAPI | |
| | | EKTISKAKGQPREPQVYTLPPSREEMTKNQVSLTCLVKGFYPSDI | |
| | | AVEWESNGQPENNYKTTPPVLDSDGSFFLYSKLTVDKSRWQQGN | |
| | | VFSCSVMHEALHNHYTQKSLSLSPGK | |
| Prolgolimab, | PD-1 | QPVLTQPLSVSVALGQTARITCGGNNIGSKNVHWYQQKPGQ | 53 |
| Forteca, | | APVLVIYRDSNRPSGIPERFSGSNSGNTATLTISRAQAGDEADY | |
| BCD-100 | | YCQVWDSSTAVFGTGTKLTVLQRTVAAPSVFIFPPSDEQLKSGT | |
| Light Chain | | ASVVCLLNNFYPREAKVQWKVDNALQSGNSQESVTEQDSKDST | |
| (VL in | | YSLSSTLTLSKADYEKHKVYACEVTHQGLSSPVTKSFNRGEC | |
| Bold) | | | |
| Balstilimab, | PD-1 | QVQLVESGGGVVQPGRSLRLSCAASGFTFSSYGMHWVRQAP | 54 |
| 1Q2QT5M7EO, | | GKGLEWVAVIWYDGSNKYYADSVKGRFTISRDNSKNTLYLQ | |
| AGEN- | | MNSLRAEDTAVYYCASNGDHWGQGTLVTVSSASTKGPSVFPL | |
| 2034, | | APCSRSTSESTAALGCLVKDYFPEPVTVSWNSGALTSGVHTFPAV | |
| AGEN- | | LQSSGLYSLSSVVTVPSSSLGTKTYTCNVDHKPSNTKVDKRVESK | |
| 2034w | | YGPPCPPCPAPEFLGGPSVFLFPPKPKDTLMISRTPEVTCVVVDVS | |
| Heavy | | QEDPEVQFNWYVDGVEVHNAKTKPREEQFNSTYRVVSVLTVLH | |
| Chain (VH | | QDWLNGKEYKCKVSNKGLPSSIEKTISKAKGQPREPQVYTLPPSQ | |
| in Bold) | | EEMTKNQVSLTCLVKGFYPSDIAVEWESNGQPENNYKTTPPVLD | |
| | | SDGSFFLYSRLTVDKSRWQEGNVFSCSVMHEALHNHYTQKSLSL | |
| | | SLG | |
| Balstilimab, | PD-1 | EIVMTQSPATLSVSPGERATLSCRASQSVSSNLAWYQQKPGQ | 55 |
| 1Q2QT5M7EO, | | APRLLIYGASTRATGIPARFSGSGSGTEFTLTISSLQSEDFAVYY | |
| AGEN- | | CQQYNNWPRTFGQGTKVEIKRTVAAPSVFIFPPSDEQLKSGTAS | |
| 2034, | | VVCLLNNFYPREAKVQWKVDNALQSGNSQESVTEQDSKDSTYS | |
| AGEN- | | LSSTLTLSKADYEKHKVYACEVTHQGLSSPVTKSFNRGEC | |
| 2034w | | | |
| Light Chain | | | |
| (VL in | | | |
| Bold) | | | |
| Dostarlimab, | PD-1 | EVQLLESGGGLVQPGGSLRLSCAASGFTFSSYDMSWVRQAPG | 56 |
| ANB-011, | | KGLEWVSTISGGGSYTYYQDSVKGRFTISRDNSKNTLYLQMN | |
| GSK- | | SLRAEDTAVYYCASPYYAMDYWGQGTTVTVSSASTKGPSVFP | |
| 4057190A, | | LAPCSRSTSESTAALGCLVKDYFPEPVTVSWNSGALTSGVHTFPA | |
| P0GVQ9A4S5, | | VLQSSGLYSLSSVVTVPSSSLGTKTYTCNVDHKPSNTKVDKRVES | |
| TSR- | | KYGPPCPPCPAPEFLGGPSVFLFPPKPKDTLMISRTPEVTCVVVDV | |
| 042, WBP- | | SQEDPEVQFNWYVDGVEVHNAKTKPREEQFNSTYRVVSVLTVL | |
| 285 Heavy | | HQDWLNGKEYKCKVSNKGLPSSIEKTISKAKGQPREPQVYTLPPS | |
| Chain (VH | | QEEMTKNQVSLTCLVKGFYPSDIAVEWESNGQPENNYKTTPPVL | |
| in Bold) | | DSDGSFFLYSRLTVDKSRWQEGNVFSCSVMHEALHNHYTQKSLS | |
| | | LSLGK | |
| Dostarlimab, | PD-1 | DIQLTQSPSFLSAYVGDRVTITCKASQDVGTAVAWYQQKPGK | 57 |
| ANB-011, | | APKLLIYWASTLHTGVPSRFSGSGSGTEFTLTISSLQPEDFATY | |
| GSK- | | YCQHYSSYPWTFGQGTKLEIKRTVAAPSVFIFPPSDEQLKSGTA | |
| 4057190A, | | SVVCLLNNFYPREAKVQWKVDNALQSGNSQESVTEQDSKDSTYS | |
| P0GVQ9A4S5, | | LSSTLTLSKADYEKHKVYACEVTHQGLSSPVTKSFNRGEC | |
| TSR- | | | |
| 042, WBP- | | | |
| 285 Light | | | |
| Chain (VL | | | |
| in Bold) | | | |
| Serplulimab, | PD-1 | QVQLVESGGGLVKPGGSLRLSCAASGFTFSNYGMSWIRQAPG | 58 |
| HLX-10 | | KGLEWSTISGGGSNIYYADSVKGRFTISRDNAKNSLYLQMNSL | |
| Heavy | | RAEDTAVYYCVSYYYGIDFWGQGTSVTVSSASKYGPSVFPLAP | |
| Chain (VH | | CSRSTSESTAALGCLVKDYFPEPVTVSWNSGALTSGVHTFPAVLQ | |
| in Bold) | | SSGLYSLSSVVTVPSSSLGTKTYTCNVDHKPSNTKVDKRVESKYG | |
| | | PPCPPCPAPEFLGGPSVFLFPPKPKDTLMISRTPEVTCVVVDVSQE | |
| | | DPEVQFNWYVDGVEVHNAKTKPREEQFNSTYRVVSVVLTVLHQ | |
| | | DWLNGKEYKCKVSNKGLPSSIEKTISKAKGQPREPQVYTLPPSQE | |
| | | EMTKNQVSLTCLVKGFYPSDIAVEWESNGQPENNYKTTPPVLDS | |
| | | DGSFFLYSRLTVDKSRWQEGNVFSCSVMHEALHNHYTQKSLSLS | |
| | | LGK | |
| | | VH CDR1: FTFSNYGMS (SEQ ID NO: 92) | |
| | | VH CDR2: TISGGGSNIY (SEQ ID NO: 93) | |
| | | VH CDR3: VSYYYGIDF (SEQ ID NO: 94) | |
| Serplulimab, | PD-1 | DIQMTQSPSSLSASVGDRVTITCKASQDVTTAVAWYQQKPGK | 59 |
| HLX-10 | | APKLLIYWASTRHTGVPSRFSGSGSGTDFTLTISSLQPEDFATY | |
| Light Chain | | YCQQHYTIPWTFGGGTKLEIKRTVAAPSVFIFPPSDEQLKSGTA | |
| (VL in | | SVVCLLNNFYPREAKVQWKVDNALQSGNSQESVTEQDSKDSTYS | |
| Bold) | | LSSTLTLSKADYEKHKVYACEVTHQGLSSPVTKSFNRGEC | |
| | | VL CDR1: KASQDVTTAVA (SEQ ID NO: 95) | |
| | | VL CDR2: WASTRHT (SEQ ID NO: 96) | |
| | | VL CDR3: QQHYTIPWT (SEQ ID NO: 97) | |
| Retifanlimab, | PD-1 | QVQLVQSGAEVKKPGASVKVSCKASGYSFTSYWMNWVRQAP | 60 |
| 2Y3T5IF01Z, | | GQGLEWIGVIHPSDSETWLDQKFKDRVTITVDKSTSTAYMEL | |
| INCMGA- | | SSLRSEDTAVYYCAREHYGTSPFAYWGQGTLVTVSSASTKGPS | |
| 00012, | | VFPLAPCSRSTSESTAALGCLVKDYFPEPVTVSWNSGALTSGVHT | |
| INCMGA- | | FPAVLQSSGLYSLSSVVTVPSSSLGTKTYTCNVDHKPSNTKVDKR | |
| 0012, MGA- | | VESKYGPPCPPCPAPEFLGGPSVFLFPPKPKDTLMISRTPEVTCVV | |
| 012 Heavy | | VDVSQEDPEVQFNWYVDGVEVHNAKTKPREEQFNSTYRVVSVL | |
| Chain (VH | | TVLHQDWLNGKEYKCKVSNKGLPSSIEKTISKAKGQPREPQVYT | |
| in Bold) | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Retifanlimab, | PD-1 | EIVLTQSPATLSLSPGERATLSCRASESVDNYGMSFMNWFQQ | 61 |
| 2Y3T5IF01Z, | | KPGQPPKLLIHAASNQGSGVPSRFSGSGSGTDFTLTISSLEPED | |
| INCMGA- | | FAVYFCQQSKEVPYTFGGGTKVEIKRTVAAPSVFIFPPSDEQLK | |
| 00012, | | SGTASVVCLLNNFYPREAKVQWKVDNALQSGNSQESVTEQDSK | |
| INCMGA- | | DSTYSLSSTLTLSKADYEKHKVYACEVTHQGLSSPVTKSFNRGEC | |
| 0012, MGA- | | | |
| 012 Light | | | |
| Chain (VL | | | |
| in Bold) | | | |
| Sasanlimab, | PD-1 | QVQLVQSGAEVKKPGASVKVSCKASGYTFTSYWINWVRQAP | 62 |
| LZZ0IC2EWP, | | GQGLEWMGNIYPGSSLTNYNEKFKNRVTMTRDTSTSTVYME | |
| PF- | | LSSLRSEDTAVYYCARLSTGTFAYWGQGTLVTVSSASTKGPSV | |
| 06801591, | | FPLAPCSRSTSESTAALGCLVKDYFPEPVTVSWNSGALTSGVHTF | |
| RN-888 | | PAVLQSSGLYSLSSVVTVPSSSLGTKTYTCNVDHKPSNTKVDKRV | |
| Heavy | | ESKYGPPCPPCPAPEFLGGPSVFLFPPKPKDTLMISRTPEVTCVVV | |
| Chain (VH | | DVSQEDPEVQFNWYVDGVEVHNAKTKPREEQFNSTYRVVSVLT | |
| in Bold) | | VLHQDWLNGKEYKCKVSNKGLPSSIEKTISKAKGQPREPQVYTLP | |
| | | PSQEEMTKNQVSLTCLVKGFYPSDIAVEWESNGQPENNYKTTPP | |
| | | VLDSDGSFFLYSRLTVDKSRWQEGNVFSCSVMHEALHNHYTQKS | |
| | | LSLSLGK | |
| Sasanlimab, | PD-1 | DIVMTQSPDSLAVSLGERATINCKSSQSLWDSGNQKNFLTWY | 63 |
| LZZ0IC2EWP, | | QQKPGQPPKLLIYWTSYRESGVPDRFSGSGSGTDFTLTISSLQ | |
| PF- | | AEDVAVYYCQNDYFYPHTFGGGTKVEIKRTVAAPSVFIFPPSDE | |
| 06801591, | | QLKSGTASVVCLLNNFYPREAKVQWKVDNALQSGNSQESVTEQ | |
| RN-888 | | DSKDSTYSLSSTLTLSKADYEKHKVYACEVTHQGLSSPVTKSFNR | |
| Light | | GEC | |
| Chain (VL | | | |
| in Bold) | | | |
| Spartalizumab, | PD-1 | EVQLVQSGAEVKKPGESLRISCKGSGYTFTTYWMHWVRQAT | 64 |
| NVP- | | GQGLEWMGNIYPGTGGSNFDEKFKNRVTITADKSTSTAYME | |
| LZV-184, | | LSSLRSEDTAVYYCTRWTTGTGAYWGQGTTVTVSSASTKGPS | |
| PDR-001, | | VFPLAPCSRSTSESTAALGCLVKDYFPEPVTVSWNSGALTSGVHT | |
| QOG25L6Z8Z | | FPAVLQSSGLYSLSSVVTVPSSSLGTKTYTCNVDHKPSNTKVDKR | |
| Heavy | | VESKYGPPCPPCPAPEFLGGPSVFLFPPKPKDTLMISRTPEVTCVV | |
| Chain (VH | | VDVSQEDPEVQFNWYVDGVEVHNAKTKPREEQFNSTYRVVSVL | |
| in Bold) | | TVLHQDWLNGKEYKCKVSNKGLPSSIEKTISKAKGQPREPQVYT | |
| | | LPPSQEEMTKNQVSLTCLVKGFYPSDIAVEWESNGQPENNYKTTP | |
| | | PVLDSDGSFFLYSRLTVDKSRWQEGNVFSCSVMHEALHNHYTQK | |
| | | SLSLSLG | |
| Spartalizumab, | PD-1 | EIVLTQSPATLSLSPGERATLSCKSSQSLLDSGNQKNFLTWYQ | 65 |
| NVP- | | QKPGQAPRLLIYWASTRESGVPSRFSGSGSGTDFTFTISSLEAE | |
| LZV-184, | | DAATYYCQNDYSYPYTFGQGTKVEIKRTVAAPSVFIFPPSDEQL | |
| PDR-001, | | KSGTASVVCLLNNFYPREAKVQWKVDNALQSGNSQESVTEQDS | |
| QOG25L6Z8Z | | KDSTYSLSSTLTLSKADYEKHKVYACEVTHQGLSSPVTKSFNRGE | |
| Light | | C | |
| Chain (VL | | | |
| in Bold) | | | |
| Cetrelimab, | PD-1 | QVQLVQSGAEVKKPGSSVKVSCKASGGTFSSYAISWVRQAPG | 66 |
| JNJ-3283, | | QGLEWMGGIIPIFDTANYAQKFQGRVTITADESTSTAYMELSS | |
| JNJ- | | LRSEDTAVYYCARPGLAAAYDTGSLDYWGQGTLVTVSSASTK | |
| 63723283, | | GPSVFPLAPCSRSTSESTAALGCLVKDYFPEPVTVSWNSGALTSG | |
| LYK98WP91F | | VHTFPAVLQSSGLYSLSSVVTVPSSSLGTKTYTCNVDHKPSNTKV | |
| Heavy | | DKRVESKYGPPCPPCPAPEFLGGPSVFLFPPKPKDTLMISRTPEVT | |
| Chain (VH | | CVVVDVSQEDPEVQFNWYVDGVEVHNAKTKPREEQFNSTYRVV | |
| in Bold) | | SVLTVLHQDWLNGKEYKCKVSNKGLPSSIEKTISKAKGQPREPQV | |
| | | YTLPPSQEEMTKNQVSLTCLVKGFYPSDIAVEWESNGQPENNYK | |
| | | TTPPVLDSDGSFFLYSRLTVDKSRWQEGNVFSCSVMHEALHNHY | |
| | | TQKSLSLSLGK | |
| Cetrelimab, | PD-1 | EIVLTQSPATLSLSPGERATLSCRASQSVRSYLAWYQQKPGQA | 67 |
| JNJ-3283, | | PRLLIYDASNRATGIPARFSGSGSGTDFTLTISSLEPEDFAVYYC | |
| JNJ- | | QQRNYWPLTFGQGTKVEIKRTVAAPSVFIFPPSDEQLKSGTASV | |
| 63723283, | | VCLLNNFYPREAKVQWKVDNALQSGNSQESVTEQDSKDSTYSLS | |
| LYK98WP91F | | STLTLSKADYEKHKVYACEVTHQGLSSPVTKSFNRGEC | |
| Light | | | |
| Chain (VL | | | |
| in Bold) | | | |
| Tebotelimab | PD-1 | DIQMTQSPSSLSASVGDRVTITCRASQDVSSVVAWYQQKPGK | 68 |
| MGD-013 | | APKLLIYSASYRYTGVPSRFSGSGSGTDFTLTISSLQPEDFATY | |
| Heavy | | YCQQHYSTPWTFGGGTKLEIKGGGSGGGGQVQLVQSGAEVKK | |
| Chain (VH | | PGASVKVSCKASGYSFTSYWMNWVRQAPGQGLEWIGVIHPSDSE | |
| in Bold) | | TWLDQKFKDRVTITVDKSTSTAYMELSSLRSEDTAVYYCAREHY | |
| | | GTSPFAYWGQGTLVTVSSGGCGGGEVAACEKEVAALEKEVAAL | |
| | | EKEVAALEKESKYGPPCPPCPAPEFLGGPSVFLFPPKPKDTLYITR | |
| | | EPEVTCVVVDVSQEDPEVQFNWYVDGVEVHNAKTKPREEQFNS | |
| | | TYRVVSVLTVLHQDWLNGKEYKCKVSNKGLPSSIEKTISKAKGQ | |
| | | PREPQVYTLPPSQEEMTKNQVSLTCLVKGFYPSDIAVEWESNGQP | |
| | | ENNYKTTPPVLDSDGSFFLYSRLTVDKSRWQEGNVFSCSVMHEA | |
| | | LHNHYTQKSLSLSLG | |
| Tebotelimab, | PD-1 | EIVLTQSPATLSLSPGERATLSCRASESVDNYGMSFMNWFQQ | 69 |
| MGD-013 | | KPGQPPKLLIHAASNQGSGVPSRFSGSGSGTDFTLTISSLEPED | |
| Light Chain | | FAVYFCQQSKEVPYTFGGGTKVEIKGGGSGGGGQVQLVQSGA | |
| (VL in | | EVKKPGASVKVSCKASGYTFTDYNMDWVRQAPGQGLEWMGDI | |
| Bold) | | NPDNGVTIYNQKFEGRVTMTTDTSTSTAYMELRSLRSDDTAVYY | |
| | | CAREADYFYFDYWGQGTTLTVSSGGGGGKVAACKEKVAALKE | |
| | | KVAALKEKVAALKE | |
| Pidilizumab, | PD-1 | QVQLVQSGSELKKPGASVKISCKASGYTFTNYGMNWVRQAP | 70 |
| CT-011, | | GQGLQWMGWINTDSGESTYAEEFKGRFVFSLDTSVNTAYLQI | |
| MDV-9300 | | TSLTAEDTGMYFCVRVGYDALDYWGQGTLVTVSSASTKGPSV | |
| Heavy | | FPLAPSSKSTSGGTAALGCLVKDYFPEPVTVSWNSGALTSGVHTF | |
| Chain (VH | | PAVLQSSGLYSLSSVVTVPSSSLGTQTYICNVNHKPSNTKVDKRV | |
| in Bold) | | EPKSCDKTHTCPPCPAPELLGGPSVFLFPPKPKDTLMISRTPEVTC | |
| | | VVVDVSHEDPEVKFNWYVDGVEVHNAKTKPREEQYNSTYRVVS | |
| | | VLTVLHQDWLNGKEYKCKVSNKALPAPIEKTISKAKGQPREPQV | |
| | | YTLPPSREEMTKNQVSLTCLVKGFYPSDIAVEWESNGQPENNYK | |
| | | TTPPVLDSDGSFFLYSKLTVDKSRWQQGNVFSCSVMHEALHNHY | |
| | | TQKSLSLSPGK | |
| Pidilizumab, | PD-1 | EIVLTQSPSSLSASVGDRVTITCSARSSVSYMHWFQQKPGKAP | 71 |
| CT-011, | | KLWIYRTSNLASGVPSRFSGSGSGTSYCLTINSLQPEDFATYYC | |
| MDV-9300 | | QQRSSFPLTFGGGTKLEIKRTVAAPSVFIFPPSDEQLKSGTASVV | |
| Light Chain | | CLLNNFYPREAKVQWKVDNALQSGNSQESVTEQDSKDSTYSLSS | |
| (VL in | | TLTLSKADYEKHKVYACEVTHQGLSSPVTKSFNRGEC | |
| Bold) | | | |
| SG-001 VH | PD-1 | QVQLVESGGGVVQPGRSLRLTCKASGLTFSSSGMHWVRQAPGK | 72 |
| | | GLEWVAVIWYDGSKRYYADSVKGRFTISRDNSKNTLFLQMNSLR | |
| | | AEDTAVYYCATNNDYWGQGTLVTVSS | |
| | | VH CDR1: GLTFSSSG (SEQ ID NO: 98) | |
| | | VH CDR2: IWYDGSKR (SEQ ID NO: 99) | |
| | | VH CDR3: ATNNDY (SEQ ID NO: 100) | |
| SG-001 VL | PD-1 | EIVLTQSPATLSLSPGERATLSCRASQSVSSYLAWYQQKPGQAPR | 73 |
| | | LLIYTASNRATGIPARFSGSGSGTDFTLTISSLEPEDFAVYYCQQYS | |
| | | NWPRTFGQGTKVEIK | |
| | | VL CDR1: RASQSVSSYLA (SEQ ID NO: 89) | |
| | | VL CDR2: TASNRAT (SEQ ID NO: 102) | |
| | | VL CDR3: QQYSNWPRT (SEQ ID NO: 103) | |
| mpLZM- | PD-1 | EVQLQQSGPVLVKPGASVKMSCKASGYTFTSYYMYWVKQSHGK | 74 |
| 009 VH | | SLEWIGGVNPSNGGTNFNEKFKSKATLTVDKSSSTAYMELNSLTS | |
| (Murine | | EDSAVYYCARRDYRYDMGFDYWGQGTTLTVSS | |
| Precursor of | | | |
| LZM-009) | | | |
| mpLZM- | PD-1 | QIVLTQSPAIMSASPGEKVTMTCRASKGVSTSGYSYLHWYQQKP | 75 |
| 009 VL | | GSSPRLLIYLASYLESGVPVRFSGSGSGTSYSLTISRMEAEDAATY | |
| (Murine | | YCQHSRELPLTFGTGTRLEIK | |
| Precursor of | | | |
| LZM-009) | | | |
| LZM-009 | PD-1 | QVQLVQSGAEVKKPGASVKVSCKASGYTFTSYYMYWVRQAPG | 76 |
| VH | | QGLEWMGGVNPSNGGTNFNEKFKSRVTITADKSTSTAYMELSSL | |
| | | RSEDTAVYYCARRDYRYDMGFDYWGQGTTVTVSS | |
| | | VH CDR1: GYTFTSYYMY (SEQ ID NO: 113) | |
| | | VH CDR2: GVNPSNGGTNFNEKFKS (SEQ ID NO: 114) | |
| | | VH CDR3: RDYRYDMGFDY (SEQ ID NO: 115) | |
| LZM-009 | PD-1 | EIVLTQSPATLSLSPGERATISCRASKGVSTSGYSYLHWYQQKPG | 77 |
| VL | | QAPRLLIYLASYLESGVPARFSGSGSGTDFTLTISSLEPEDFATYYC | |
| | | QHSRELPLTFGTGTKVEIK | |
| | | VL CDR1: RASKGVSTSGYSYLH (SEQ ID NO: 83) | |
| | | VL CDR2: LASYLE (SEQ ID NO: 117) | |
| | | VL CDR3: QHSRELPLT (SEQ ID NO: 118) | |
| Budigalimab, | PD-1 | EIQLVQSGAEVKKPGSSVKVSCKASGYTFTHYGMNWVRQAP | 78 |
| 6VDO4TY3OO, | | GQGLEWVGWVNTYTGEPTYADDFKGRLTFTLDTSTSTAYME | |
| ABBV- | | LSSLRSEDTAVYYCTREGEGLGFGDWGQGTTVTVSSASTKGP | |
| 181, PR- | | SVFPLAPSSKSTSGGTAALGCLVKDYFPEPVTVSWNSGALTSGVH | |
| 1648817 | | TFPAVLQSSGLYSLSSVVTVPSSSLGTQTYICNVNHKPSNTKVDK | |
| Heavy | | KVEPKSCDKTHTCPPCPAPEAAGGPSVFLFPPKPKDTLMISRTPEV | |
| Chain (VH | | TCVVVDVSHEDPEVKFNWYVDGVEVHNAKTKPREEQYNSTYRV | |
| in Bold) | | VSVLTVLHQDWLNGKEYKCKVSNKALPAPIEKTISKAKGQPREP | |
| | | QVYTLPPSREEMTKNQVSLTCLVKGFYPSDIAVEWESNGQPENN | |
| | | YKTTPPVLDSDGSFFLYSKLTVDKSRWQQGNVFSCSVMHEALHN | |
| | | HYTQKSLSLSPGK | |
| Budigalimab, | PD-1 | DVVMTQSPLSLPVTPGEPASISCRSSQSIVHSHGDTYLEWYLQ | 79 |
| 6VDO4TY3OO, | | KPGQSPQLLIYKVSNRFSGVPDRFSGSGSGTDFTLKISRVEAED | |
| ABBV- | | VGVYYCFQGSHIPVTFGQGTKLEIKRTVAAPSVFIFPPSDEQLKS | |
| 181, PR- | | GTASVVCLLNNFYPREAKVQWKVDNALQSGNSQESVTEQDSKD | |
| 1648817 | | STYSLSSTLTLSKADYEKHKVYACEVTHQGLSSPVTKSFNRGEC | |
| Light Chain | | | |
| (VL in | | | |
| Bold) | | | |
| PD-1-Fc- | PD-1 | MQIPQAPWPWWAVLQLGWRPGWFLDSPDRPWNPPTFSPALLVV | 104 |
| OX40L | | TEGDNATFTCSFSNTSESFVLNWYRMSPSNQTDKLAAFPEDRSQP | |
| (Code), SL- | | GQDCRFRVTQLPNGRDFHMSVVRARRNDSGTYLCGAISLAPKAQ | |
| 279252 | | IKESLRAELRVTERRAEVPTAHPSPSPRPAGQFQSKYGPPCPSCPA | |
| (Code), | | PEFLGGPSVFLFPPKPKDTLMISRTPEVTCWVDVSQEDPEVQFNW | |
| TAK-252 | | YVDGVEVHNAKTKPREEQFNSTYRVVSVLTVLHQDWLSGKEYK | |
| (Code) | | CKVSSKGLPSSIEKTISNATGQPREPQVYTLPPSQEEMTKNQVSLT | |
| | | CLVKGFYPSDIAVEWESNGQPENNYKTTPPVLDSDGSFFLYSRLT | |
| | | VDKSSWQEGNVFSCSVMHEALHNHYTQKSLSLSLGKIEGRMDQ | |
| | | VSHRYPRIQSIKVQFTEYKKEKGFILTSQKEDEIMKVQNNSVIINC | |
| | | DGFYLISLKGYFSQEVNISLHYQKDEEPLFQLKKVRSVNSLMVAS | |
| | | LTYKDKVYLNVTTDNTSLDDFHVNGGELILIHQNPGEFCVLMQIP | |
| | | QAPWPWWAVLQLGWRPGWFLDSPDRPWNPPTFSPALLVVTEGD | |
| | | NATFTCSFSNTSESFVLNWYRMSPSNQTDKLAAFPEDRSQPGQDC | |
| | | RFRVTQLPNGRDFHMSVVRARRNDSGTYLCGAISLAPKAQIKESL | |
| | | RAELRVTERRAEVPTAHPSPSPRPAGQFQQVSHRYPRIQSIKVQFT | |
| | | EYKKEKGFILTSQKEDEIMKVQNNSVIINCDGFYLISLKGYFSQEV | |
| | | NISLHYQKDEEPLFQLKKVRSVNSLMVASLTYKDKVYLNVTTDN | |
| | | TSLDDFHVNGGELILIHQNPGEFCVL | |
| Anti-PD-1 | PD-1 | DVQLVESGGG VVQPGGSLRL SCAASGSIAS | 1 |
| VHH 1 | | IHAMGWFRQA PGKEREFVAV ITWSGGITYY ADSVKGRFTI | |
| | | SRDNSKNTVY LQMNSLRPED TALYYCAGDK HQSSWYDYWG | |
| | | QGTLVTVSS (SEQ ID NO: 1) | |
| | | VH CDR1: GSIASIHA (SEQ ID NO: 2) | |
| | | VH CDR2: ITWSGGIT (SEQ ID NO: 3) | |
| | | VH CDR3: AGDKHQSSWYDY (SEQ ID NO: 4) | |
| Anti-PD-1 | PD-1 | EVQLVESGGGLVKPGGSLRLSCAASGFTFSDESMTWMRQAPGKG | 5 |
| VHH 2 | | LEWVSYISSGGGVKFYADSVKGRFTISRDNAKNSLYLQMNSLRA | |
| | | EDTAVYYCAREAPLRLGESPHDAFDISGQGTMVTVSS (SEQ ID | |
| | | NO: 5) | |
| | | VH CDR1: DESMT (SEQ ID NO: 6) | |
| | | VH CDR2: YISSGGGVKFYADSVKG (SEQ ID NO: 7) | |
| | | VH CDR3: EAPLRLGESPHDAFDI (SEQ ID NO: 8) | |
| Anti-PD-1 | PD-1 | QVQLQESGGGSVQAGGSLRLSCVASQYTYNTVGWFRQAPGKER | 13 |
| VHH 3 | | EGVAGIYNGGDQTYYSESAKGRFTISQDNAKRTVYLQMNSLKPE | |
| | | DTAMYYCAAGRLIVSGRWSMTKEEYQYWGQGTQVTVSS (SEQ | |
| | | ID NO: 13) | |
| | | VH CDR1: QYTYNT (SEQ ID NO: 14) | |
| | | VH CDR2: IYNGGDQT (SEQ ID NO: 15) | |
| | | VH CDR3: AAGRLIVSGRWSMTKEEYQY (SEQ ID NO: 16) | |
| Anti-PD-1 | PD-1 | EVQLVESGGGEVQPGGSLRLSCAASGSITGANTMGWYRQAPGK | 17 |
| VHH 4 | | QRDLVALIGNYVTHYAESVKGRFTISRDNAKNTVYLQMSSLRAE | |
| | | DTAVYYCYLYTDNLGTSWGQGTLVTVKP (SEQ ID NO: 17) | |
| | | VH CDR1: ANTMG (SEQ ID NO: 18) | |
| | | VH CDR2: LIGNYVTHYAESVKG (SEQ ID NO: 19) | |
| | | VH CDR3: YTDNLGTS (SEQ ID NO: 20) | |
| Anti-PD-1 | PD-1 | EVQLVESGGGLVQPGGSLRLSCAASGFAFSSYDMSWVRQAPG | 105 |
| scFv from | | KGLDWVATISGGGRYTYYPDSVKGRFTISRDNSKNNLYLQMN | |
| Ivonescimab | | SLRAEDTALYYCANRYGEAWFAYWGQGTLVTVSSGGGGSGG | |
| (VH in bold, | | GGSGGGGSGGGGSDIQMTQSPSSMSASVGDRVTFTCRASQDINT | |
| VL in | | YLSWFQQKPGKSPKTLIYRANRLVSGVPSRFSGSGSGQDYTLTISS | |
| bold + italics) | | LQPEDMATYYCLQYDEFPLTFGAGTKLELKR | |
| Flipped | PD-1 | EVQLVESGGGLVQPGGSLRLSCAASGFAFSSYDMSWVRQAPG | 106 |
| Ivonescimab | | KGLDWVATISGGGRYTYYPDSVKGRFTISRDNSKNNLYLQMN | |
| HC, no scFv | | SLRAEDTALYYCANRYGEAWFAYWGQGTLVTVSSASTKGPSV | |
| targeting | | FPLAPSSKSTSGGTAALGCLVKDYFPEPVTVSWNSGALTSGVHTF | |
| VEGF (VH | | PAVLQSSGLYSLSSVVTVPSSSLGTQTYICNVNHKPSNTKVDKKV | |
| in bold) | | EPKSCDKTHTCPPCPAPEAAGGPSVFLFPPKPKDTLMISRTPEVTC | |
| | | VVVDVSHEDPEVKFNWYVDGVEVHNAKTKPREEQYNSTYRVVS | |
| | | VLTVLHQDWLNGKEYKCKVSNKALPAPIEKTISKAKGQPREPQV | |
| | | YTLPPSRDELTKNQVSLTCLVKGFYPSDIAVEWESNGQPENNYKT | |
| | | TPPVLDSDGSFFLYSKLTVDKSRWQQGNVFSCSVMHEALHNHYT | |
| | | QKSLSLSPGK | |
| Flipped | PD-1 | EIVLTQSPATLSLSPGERATISCRASKGVSTSGYSYLHWYQQK | 107 |
| Ivonescimab | | PGQAPRLLIYLASYLESGVPARFSGSGSGTDFTLTISSLEPEDF | |
| LC (VL in | | ATYYCQHSRELPLTFGTGTKVEIKRTVAAPSVFIFPPSDEQLKS | |
| bold) | | GTASVVCLLNNFYPREAKVQWKVDNALQSGNSQESVTEQDSKD | |
| | | STYSLSSTLTLSKADYEKHKVYACEVTHQGLSSPVTKSFNRGEC | |
| pembrolizumab | PD-1 | QVQLVQSGVEVKKPGASVKVSCKASGYTFTNYYMYWVRQAP | 108 |
| scFv | | GQGLEWMGGINPSNGGTNFNEKFKNRVTLTTDSSTTTAYME | |
| VH-VL | | LKSLQFDDTAVYYCARRDYRFDMGFDYWGQGTTVTVSSGGG | |
| (VH in bold, | | GSGGGGSGGGGSGGGGSEIVLTQSPATLSLSPGERATLSCRASKG | |
| VL in | | VSTSGYSYLHWYQQKPGQAPRLLIYLASYLESGVPARFSGSGSGT | |
| bold + italics) | | DFTLTISSLEPEDFAVYYCQHSRDLPLTFGGGTKVEIK | |
| pembrolizumab | PD-1 | EIVLTQSPATLSLSPGERATLSCRASKGVSTSGYSYLHWYQQKPGQ | 109 |
| scFv | | APRLLIYLASYLESGVPARFSGSGSGTDFTLTISSLEPEDFAVYYCQ | |
| VL-VH | | HSRDLPLTFGGGTKVEIKGGGGSGGGGSGGGGSGGGGSQVQLV | |
| (VH in bold, | | QSGVEVKKPGASVKVSCKASGYTFTNYYMYWVRQAPGQGLE | |
| VL in | | WMGGINPSNGGTNFNEKFKNRVTLTTDSSTTTAYMELKSLQF | |
| bold + italics) | | DDTAVYYCARRDYRFDMGFDYWGQGTTVTVSS | |
| LZM009 | PD-1 | QVQLVQSGAEVKKPGASVKVSCKASGYTFTSYYMYWVRQAP | 110 |
| scFv VH- | | GQGLEWMGGVNPSNGGTNFNEKFKSRVTITADKSTSTAYME | |
| VL | | LSSLRSEDTAVYYCARRDYRYDMGFDYWGQGTTVTVSSGGG | |
| (VH in bold, | | GSGGGGSGGGGSGGGGSEIVLTQSPATLSLSPGERATISCRASKG | |
| VL in | | VSTSGYSYLHWYQQKPGQAPRLLIYLASYLESGVPARFSGSGSGT | |
| bold + italics) | | DFTLTISSLEPEDFATYYCQHSRELPLTFGTGTKVEI | |
| LZM009 | PD-1 | EIVLTQSPATLSLSPGERATISCRASKGVSTSGYSYLHWYQQKPGQ | 111 |
| scFv VL- | | APRLLIYLASYLESGVPARFSGSGSGTDFTLTISSLEPEDFATYYCQ | |
| VH | | HSRELPLTFGTGTKVEIKGGGGSGGGGSGGGGSGGGGSQVQLV | |
| (VH in bold, | | QSGAEVKKPGASVKVSCKASGYTFTSYYMYWVRQAPGQGLE | |
| VL in | | WMGGVNPSNGGTNFNEKFKSRVTITADKSTSTAYMELSSLRS | |
| bold + italics) | | EDTAVYYCARRDYRYDMGFDYWGQGTTVTVSS | |

TABLE 1B

| Antibody | | | |
|---|---|---|---|
| or Ag- | | | SEQ |
| binding | Antigen | | ID |
| fragment | Bound | Sequence | NO |
| Anti PD1 | PD-1 | EVQLVESGGGLVQPGGSLRLSCAVSGNIYNRNFMGWFRQAPGK | |
| VHH5 | | GLEGVSAIYTGTSRTYYADSVKGRFTISRDNSKNTVYLQMNSLRA | |
| | | EDTAVYYCAADLREGFWDTGVWNTWGQGTLVTVSS (SEQ ID | |
| | | NO: 287) | |
| | | VH CDR1: RNFMG (SEQ ID NO: 288) | |
| | | VH CDR2: AIYTGTSRTYYADSVKG (SEQ ID NO: 289) | |
| | | VH CDR3: DLREGFWDTGVWNT (SEQ ID NO: 290) | |
| Anti-PD1 X | PD-1 | EVQLVESGGGLVQPGGSLRLSCAASGFAFSNYYMYWVRQAPGK | |
| VH | | GLEWMGGINPSNGGTNFNEKFKNRVTISRDNSKNNLYLQMNSLR | |
| | | AEDTALYYCARRDYRFDMGFDYWGQGTLVTVSS (SEQ ID NO: 9) | |
| Anti-PD1 X | PD-1 | DIQMTQSPSSMSASVGDRVTFTCRASKGVSTSGYSYLHWFQQKP | |
| VL | | GKSPKTLIYLASYLESGVPSRFSGSGSGQDYTLTISSLQPEDMATY | |
| | | YCQHSRDLPLTFGAGTKLELKR (SEQ ID NO: 10) | |
| Anti-PD1 Y | PD-1 | EVQLVESGGGLVQPGGSLRLSCAASGFAFSSYYMYWVRQAPGK | |
| VH | | GLEWMGGVNPSNGGTNFNEKFKSRVTISRDNSKNNLYLQMNSL | |
| | | RAEDTALYYCARRDYRYDMGFDYWGQGTLVTVSS (SEQ ID NO: | |
| | | 11) | |
| Anti-PD1 Y | PD-1 | DIQMTQSPSSMSASVGDRVTFTCRASKGVSTSGYSYLHWFQQKP | |
| VL | | GKSPKTLIYLASYLESGVPSRFSGSGSGQDYTLTISSLQPEDMATY | |
| | | YCQHSRELPLTFGAGTKLELKR (SEQ ID NO: 12) | |

In some embodiments, the anti-PD-1 binding domain is one provided in Table 1C, or a derivative thereof. In some embodiments, the anti-PD-1 binding domain is one which comprises the CDRs of a VHH provided in Table 1C (e.g., those of VHH 74, 62, 70, 76, 84, or 178). In some embodiments, the anti-PD-1 binding domain is one which comprises the CDRs of a VHH provided in Table 1C and comprises an amino acid sequence having at least 80%, 85%, 90%, 95%, 96%, 97%, 98%, 99%, or 100% identity to the corresponding full-length VHH sequence (e.g., VHH 74, 62, 70, 76, 84, or 178). In some embodiments, the anti-PD-1 binding domain is one of the VHHs provided in Table 1C. sequence (e.g., VHH 74, 62, 70, 76, 84, or 178).

TABLE 1C
Exemplary anti-PD-1 VHH binding domains

| | | Full | | | | | | |
|---|---|---|---|---|---|---|---|---|
| | | Length | | | | | | |
| anti | | VHH | | CDR1 | | CDR2 | | CDR3 |
| PD-1 | | SEQ | | SEQ | | SEQ | | SEQ |
| VHH No. | Sequence | ID NO | CDR1 | ID NO | CDR2 | ID NO | CDR3 | ID NO |
| VH | EVQLVESGGGLV | 501 | GFDF | 502 | INAP | 503 | ARDEAV | 504 |
| H45 | QPGGSLRLSCAAS | | STTW | | GSET | | AGYHW | |
| | GFDFSTTWMGWL | | | | | | WFDP | |
| | RQAPGKEREFVA | | | | | | | |
| | AINAPGSETYYAD | | | | | | | |
| | SVKGRFTISRDNS | | | | | | | |
| | KNTLYLQMNSLR | | | | | | | |
| | PEDTAVYYCARD | | | | | | | |
| | EAVAGYHWWFD | | | | | | | |
| | PWGQGTLVTVSS | | | | | | | |
| VH | EVQLVESGGGLV | 505 | GFTV | 506 | INDE | 507 | ARVVSG | 508 |
| H46 | QPGGSLRLSCAAS | | SDYD | | GTTT | | QQLVFP | |
| | GFTVSDYDMAWY | | | | | | LDY | |
| | RQAPGKGRELVA | | | | | | | |
| | GINDEGTTTSYAD | | | | | | | |
| | SVKGRFTISRDNA | | | | | | | |
| | KNTLYLQMNSLR | | | | | | | |
| | PEDTAVYYCARV | | | | | | | |
| | VSGQQLVFPLDY | | | | | | | |
| | WGQGSLVTVSS | | | | | | | |
| VH | DVQLVESGGGLV | 509 | GLPF | 510 | ISGSS | 511 | ATSGYS | 512 |
| H47 | QPGGSLRLSCAAS | | SDYS | | ITT | | YVAGG | |
| | GLPFSDYSMGWF | | | | | | MDV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYVAGGMDVWG | | | | | | | |
| | QGTTVTVSS | | | | | | | |
| VH | EVQLVESGGGLV | 513 | GFVF | 514 | ISAP | 515 | AREDSS | 516 |
| H48 | QPGGSLRLSCAAS | | SDHA | | VGVT | | GYLDWF | |
| | GFVFSDHAMGWF | | | | | | DP | |
| | RQAPGKGRELVA | | | | | | | |
| | AISAPVGVTYYPD | | | | | | | |
| | SVEGRFTISRDNA | | | | | | | |
| | KRMVYLQMNSLR | | | | | | | |
| | AEDTAVYYCARE | | | | | | | |
| | DSSGYLDWFDPW | | | | | | | |
| | GQGTLVTVSS | | | | | | | |
| VH | DVQLVESGGGLV | 517 | GLSF | 518 | ISGSS | 511 | ASLGPT | 520 |
| H49 | QPGGSLRLSCAAS | | SDYS | | ITT | | YYYDSS | |
| | GLSFSDYSMGWF | | | | | | GDDY | |
| | RQAPGKEREFVA | | | | | | | |
| | GISGSSITTYYADS | | | | | | | |
| | VKGRFTISRDNSK | | | | | | | |
| | NTVYLQMNSLRP | | | | | | | |
| | EDTAVYYCASLG | | | | | | | |
| | PTYYYDSSGDDY | | | | | | | |
| | WGQGTLVTVSS | | | | | | | |
| VH | EVQLVESGGGLV | 521 | GFTV | 522 | IDAS | 523 | ARHVW | 524 |
| H50 | QPGGSLRLSCAAS | | NDY | | GTKT | | GDHGG | |
| | GFTVNDYDMAW | | D | | | | WYPLDY | |
| | YRQAPGKGRELV | | | | | | | |
| | AGIDASGTKTSYA | | | | | | | |
| | DSVKGRFTISRDN | | | | | | | |
| | AKNTLYLQMNSL | | | | | | | |
| | RPEDTAVYYCAR | | | | | | | |
| | HVWGDHGGWYP | | | | | | | |
| | LDYWGQGTLVTV | | | | | | | |
| | SS | | | | | | | |
| VH | EVQLVESGGGLV | 525 | GFSV | 526 | LTES | 527 | ARVVVS | 528 |
| H51 | QPGGSLRLSCAAS | | NDY | | GGST | | DSSGYY | |
| | GFSVNDYDMAW | | D | | | | YPLDY | |
| | YRQAPGKGRELV | | | | | | | |
| | AGLTESGGSTSYA | | | | | | | |
| | DSVKGRFTISRDN | | | | | | | |
| | AKNTLYLQMNSL | | | | | | | |
| | RPEDTAVYYCAR | | | | | | | |
| | VVVSDSSGYYYPL | | | | | | | |
| | DYWGQGTLVTVS | | | | | | | |
| | S | | | | | | | |
| VH | EVQLVESGGGLV | 529 | GISFS | 530 | ISSK | 531 | VGGEQ | 532 |
| H52 | QPGGSLRLSCAAS | | DYG | | GGAT | | WLEDN | |
| | GISFSDYGMGWF | | | | | | | |
| | RQAPGKGRELVA | | | | | | | |
| | AISSKGGATYYPD | | | | | | | |
| | SVEGRFTISRDNA | | | | | | | |
| | KRMVYLQMNSLR | | | | | | | |
| | AEDTAVYYCVGG | | | | | | | |
| | EQWLEDNWGQG | | | | | | | |
| | TLVTVSS | | | | | | | |
| VH | EVQLVESGGGLV | 533 | GFTF | 534 | ISGP | 535 | AKEEPA | 536 |
| H53 | QPGGSLRLSCAAS | | SDYT | | GGIT | | GYSSGW | |
| | GFTFSDYTMGWF | | | | | | YGFDY | |
| | RQAPGKGRELVA | | | | | | | |
| | AISGPGGITYYPDS | | | | | | | |
| | VEGRFTISRDNAK | | | | | | | |
| | RMVYLQMNSLRA | | | | | | | |
| | EDTAVYYCAKEE | | | | | | | |
| | PAGYSSGWYGFD | | | | | | | |
| | YWGQGTLVTVSS | | | | | | | |
| VH | EVQLVESGGGLV | 537 | GFTF | 538 | IGSS | 539 | ARDELIS | 540 |
| H54 | QPGGSLRLSCAAS | | GDT | | GSDT | | GYAWY | |
| | GFTFGDTWMGWL | | W | | | | FDN | |
| | RQAPGKEREFVA | | | | | | | |
| | AIGSSGSDTYYAD | | | | | | | |
| | SVKGRFTISRDNS | | | | | | | |
| | KNTLYLQMNSLR | | | | | | | |
| | PEDTAVYYCARD | | | | | | | |
| | ELISGYAWYFDN | | | | | | | |
| | WGQGTLVTVSS | | | | | | | |
| VH | EVQLVESGGGLV | 541 | GLDF | 542 | ISAP | 543 | AALGDY | 544 |
| H55 | QPGGSLRLSCAAS | | HNY | | GGAT | | DDY | |
| | GLDFHNYAMGWF | | A | | | | | |
| | RQAPGKGRELVA | | | | | | | |
| | AISAPGGATYYPD | | | | | | | |
| | SVEGRFTISRDNA | | | | | | | |
| | KRMVYLQMNSLR | | | | | | | |
| | AEDTAVYYCAAL | | | | | | | |
| | GDYDDYWGQGT | | | | | | | |
| | LVTVSS | | | | | | | |
| VH | DVQLVESGGGLV | 545 | GFTF | 546 | IMGS | 547 | ASQYSY | 548 |
| H56 | QPGGSLRLSCAAS | | SDYD | | GGTT | | GLSYFD | |
| | GFTFSDYDMGWF | | | | | | Y | |
| | RQAPGKEREFVA | | | | | | | |
| | AIMGSGGTTYYA | | | | | | | |
| | DSVKGRFTISRDN | | | | | | | |
| | SKNTVYLQMNSL | | | | | | | |
| | RPEDTAVYYCAS | | | | | | | |
| | QYSYGLSYFDYW | | | | | | | |
| | GQGTLVTVSS | | | | | | | |
| VH | DVQLVESGGGLV | 549 | GIPFS | 550 | ISGSS | 511 | ASLGYS | 552 |
| H57 | QPGGSLRLSCAAS | | DYS | | ITT | | YGYGGF | |
| | GIPFSDYSMGWFR | | | | | | DY | |
| | QAPGKEREFVAGI | | | | | | | |
| | SGSSITTYYADSV | | | | | | | |
| | KGRFTISRDNSKN | | | | | | | |
| | TVYLQMNSLRPE | | | | | | | |
| | DTAVYYCASLGY | | | | | | | |
| | SYGYGGFDYWGQ | | | | | | | |
| | GTLVTVSS | | | | | | | |
| VH | EVQLVESGGGLV | 553 | GFKF | 554 | ISNK | 555 | AGGSG | 556 |
| H58 | QPGGSLRLSCAAS | | EEYA | | GGAT | | WLTDY | |
| | GFKFEEYAMGWF | | | | | | | |
| | RQAPGKGRELVA | | | | | | | |
| | AISNKGGATYYPD | | | | | | | |
| | SVEGRFTISRDNA | | | | | | | |
| | KRMVYLQMNSLR | | | | | | | |
| | AEDTAVYYCAGG | | | | | | | |
| | SGWLTDYWGRGT | | | | | | | |
| | LVTVSS | | | | | | | |
| VH | EVQLVESGGGLV | 557 | GFAF | 558 | ISAP | 543 | TTGEQW | 560 |
| H59 | QPGGSLRLSCAAS | | GDY | | GGAT | | LDDY | |
| | GFAFGDYAMGWF | | A | | | | | |
| | RQAPGKGRELVA | | | | | | | |
| | AISAPGGATYYPD | | | | | | | |
| | SVEGRFTISRDNA | | | | | | | |
| | KRMVYLQMNSLR | | | | | | | |
| | AEDTAVYYCTTG | | | | | | | |
| | EQWLDDYWGQG | | | | | | | |
| | TLVTVSS | | | | | | | |
| VH | EVQLVESGGGLV | 561 | GFDF | 562 | ISGY | 563 | AGGSG | 564 |
| H60 | QPGGSLRLSCAAS | | DNY | | GGAT | | WLSDS | |
| | GFDFDNYGMGWF | | G | | | | | |
| | RQAPGKGRELVA | | | | | | | |
| | AISGYGGATYYPD | | | | | | | |
| | SVEGRFTISRDNA | | | | | | | |
| | KRMVYLQMNSLR | | | | | | | |
| | AEDTAVYYCAGG | | | | | | | |
| | SGWLSDSWGQGT | | | | | | | |
| | MVTVSS | | | | | | | |
| VH | EVQLVESGGGLV | 565 | GGTF | 566 | ISGP | 567 | AGGQG | 568 |
| H61 | QPGGSLRLSCAAS | | SDYA | | GGAT | | WLTDY | |
| | GGTFSDYAMGWF | | | | | | | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCAGG | | | | | | | |
| | QGWLTDYWGQG | | | | | | | |
| | TLVTVSS | | | | | | | |
| VH | DVQLVESGGGLV | 569 | GLSF | 570 | ISGSS | 511 | ASLGLT | 572 |
| H62 | QPGGSLRLSCAAS | | SEYS | | ITT | | YGDSDQ | |
| | GLSFSEYSMGWF | | | | | | FDY | |
| | RQAPGKEREFVA | | | | | | | |
| | GISGSSITTYYADS | | | | | | | |
| | VKGRFTISRDNSK | | | | | | | |
| | NTVYLQMNSLRP | | | | | | | |
| | EDTAVYYCASLG | | | | | | | |
| | LTYGDSDQFDYW | | | | | | | |
| | GQGTLVTVSS | | | | | | | |
| VH | EVQLVESGGGLV | 573 | GLTIS | 574 | ISGP | 567 | TTGFYS | 576 |
| H63 | QPGGSLRLSCAAS | | DYA | | GGAT | | LEDY | |
| | GLTISDYAMGWF | | | | | | | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCTTG | | | | | | | |
| | FYSLEDYWGQGT | | | | | | | |
| | LVTVSS | | | | | | | |
| VH | EVQLVESGGGLV | 577 | GFPF | 578 | ISGH | 579 | AGLGDY | 580 |
| H64 | QPGGSLRLSCAAS | | SDYA | | GGAT | | EDY | |
| | GFPFSDYAMGWF | | | | | | | |
| | RQAPGKGRELVA | | | | | | | |
| | AISGHGGATYYPD | | | | | | | |
| | SVEGRFTISRDNA | | | | | | | |
| | KRMVYLQMNSLR | | | | | | | |
| | AEDTAVYYCAGL | | | | | | | |
| | GDYEDYWGQGTL | | | | | | | |
| | VTVSS | | | | | | | |
| VH | EVQLVESGGGLV | 581 | GFDF | 582 | ISNK | 555 | AGGSYL | 584 |
| H65 | QPGGSLRLSCAAS | | SNYG | | GGAT | | EDY | |
| | GFDFSNYGMGWF | | | | | | | |
| | RQAPGKGRELVA | | | | | | | |
| | AISNKGGATYYPD | | | | | | | |
| | SVEGRFTISRDNA | | | | | | | |
| | KRMVYLQMNSLR | | | | | | | |
| | AEDTAVYYCAGG | | | | | | | |
| | SYLEDYWGQGTL | | | | | | | |
| | VTVSS | | | | | | | |
| VH | EVQLVESGGGLV | 585 | GLNF | 586 | ISAG | 587 | AGGSG | 588 |
| H66 | QPGGSLRLSCAAS | | DNY | | SGAT | | WLTDS | |
| | GLNFDNYGMGWF | | G | | | | | |
| | RQAPGKGRELVA | | | | | | | |
| | AISAGSGATYYPD | | | | | | | |
| | SVEGRFTISRDNA | | | | | | | |
| | KRMVYLQMNSLR | | | | | | | |
| | AEDTAVYYCAGG | | | | | | | |
| | SGWLTDSWGQGS | | | | | | | |
| | LVTVSS | | | | | | | |
| VH | EVQLVESGGGLV | 589 | GFSF | 590 | ISAP | 591 | AREDSS | 592 |
| H67 | QPGGSLRLSCAAS | | SDAA | | GGVT | | GWINWF | |
| | GFSFSDAAMGWF | | | | | | DP | |
| | RQAPGKGRELVA | | | | | | | |
| | AISAPGGVTYYPD | | | | | | | |
| | SVEGRFTISRDNA | | | | | | | |
| | KRMVYLQMNSLR | | | | | | | |
| | AEDTAVYYCARE | | | | | | | |
| | DSSGWINWFDPW | | | | | | | |
| | GQGTLVTVSS | | | | | | | |
| VH | EVQLVESGGGLV | 593 | GFDF | 594 | ISNK | 555 | ARLEDT | 596 |
| H68 | QPGGSLRLSCAAS | | NDH | | GGAT | | ATT | |
| | GFDFNDHAMGWF | | A | | | | | |
| | RQAPGKGRELVA | | | | | | | |
| | AISNKGGATYYPD | | | | | | | |
| | SVEGRFTISRDNA | | | | | | | |
| | KRMVYLQMNSLR | | | | | | | |
| | AEDTAVYYCARL | | | | | | | |
| | EDTATTWGQGTL | | | | | | | |
| | VTVSS | | | | | | | |
| VH | EVQLVESGGGLV | 597 | GFPF | 578 | ISGL | 599 | AGGEDY | 600 |
| H69 | QPGGSLRLSCAAS | | SDYA | | GGAT | | IIDH | |
| | GFPFSDYAMGWF | | | | | | | |
| | RQAPGKGRELVA | | | | | | | |
| | AISGLGGATYYPD | | | | | | | |
| | SVEGRFTISRDNA | | | | | | | |
| | KRMVYLQMNSLR | | | | | | | |
| | AEDTAVYYCAGG | | | | | | | |
| | EDYIIDHWGQGVL | | | | | | | |
| | VTVSS | | | | | | | |
| VH | DVQLVESGGGLV | 601 | GITFS | 602 | ISGSS | 511 | ATAGLS | 604 |
| H70 | QPGGSLRLSCAAS | | DYS | | ITT | | GSYGGL | |
| | GITFSDYSMGWFR | | | | | | DY | |
| | QAPGKEREFVAGI | | | | | | | |
| | SGSSITTYYADSV | | | | | | | |
| | KGRFTISRDNSKN | | | | | | | |
| | TVYLQMNSLRPE | | | | | | | |
| | DTAVYYCATAGL | | | | | | | |
| | SGSYGGLDYWGQ | | | | | | | |
| | GTLVTVSS | | | | | | | |
| VH | EVQLVESGGGLV | 605 | GFDF | 606 | ISGP | 567 | AKLPVT | 608 |
| H71 | QPGGSLRLSCAAS | | NDFA | | GGAT | | TEGPGD | |
| | GFDFNDFAMGWF | | | | | | AFDI | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCAKL | | | | | | | |
| | PVTTEGPGDAFDI | | | | | | | |
| | WGQGTMVTVSS | | | | | | | |
| VH | DVQLVESGGGLV | 609 | GFDF | 610 | ISAP | 591 | TTDFVS | 612 |
| H72 | QPGGSLRLSCAAS | | SDAA | | GGVT | | GYLDY | |
| | GFDFSDAAMGWF | | | | | | | |
| | RQAPGKGRELVA | | | | | | | |
| | AISAPGGVTYYPD | | | | | | | |
| | SVEGRFTISRDNA | | | | | | | |
| | KRMVYLQMNSLR | | | | | | | |
| | PEDTAVYYCTTDF | | | | | | | |
| | VSGYLDYWGQGI | | | | | | | |
| | LVTVSS | | | | | | | |
| VH | EVQLVESGGGLV | 613 | GVDF | 614 | ISSY | 615 | AVLGDY | 616 |
| H73 | QPGGSLRLSCAAS | | SNYA | | GGAT | | EDY | |
| | GVDFSNYAMGWF | | | | | | | |
| | RQAPGKGRELVA | | | | | | | |
| | AISSYGGATYYPD | | | | | | | |
| | SVEGRFTISRDNA | | | | | | | |
| | KRMVYLQMNSLR | | | | | | | |
| | AEDTAVYYCAVL | | | | | | | |
| | GDYEDYWGQGTL | | | | | | | |
| | VTVSS | | | | | | | |
| VH | EVQLVESGGGLV | 617 | GSAF | 618 | ISGP | 567 | AALGDY | 620 |
| H74 | QPGGSLRLSCAAS | | GTYA | | GGAT | | FDY | |
| | GSAFGTYAMGWF | | | | | | | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCAAL | | | | | | | |
| | GDYFDYWGQGTL | | | | | | | |
| | VTVSS | | | | | | | |
| VH | EVQLVESGGGLV | 621 | GLTF | 622 | ISGP | 567 | AALPDN | 624 |
| H75 | QPGGSLRLSCAAS | | SDYG | | GGAT | | DYGDYL | |
| | GLTFSDYGMGWF | | | | | | | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCAAL | | | | | | | |
| | PDNDYGDYLWGQ | | | | | | | |
| | GTMVTVSS | | | | | | | |
| VH | DVQLVESGGGLV | 625 | GISFS | 626 | ISGSS | 511 | ASLGLS | 628 |
| H76 | QPGGSLRLSCAAS | | DYS | | ITT | | QYSGYD | |
| | GISFSDYSMGWFR | | | | | | EDY | |
| | QAPGKEREFVAGI | | | | | | | |
| | SGSSITTYYADSV | | | | | | | |
| | KGRFTISRDNSKN | | | | | | | |
| | TVYLQMNSLRPE | | | | | | | |
| | DTAVYYCASLGL | | | | | | | |
| | SQYSGYDEDYWG | | | | | | | |
| | QGTLVTVSS | | | | | | | |
| VH | EVQLVESGGGLV | 629 | GFNF | 630 | ISGY | 563 | AGGSSS | 632 |
| H77 | QPGGSLRLSCAAS | | DEFG | | GGAT | | VEDY | |
| | GFNFDEFGMGWF | | | | | | | |
| | RQAPGKGRELVA | | | | | | | |
| | AISGYGGATYYPD | | | | | | | |
| | SVEGRFTISRDNA | | | | | | | |
| | KRMVYLQMNSLR | | | | | | | |
| | AEDTAVYYCAGG | | | | | | | |
| | SSSVEDYWGQGT | | | | | | | |
| | LVTVSS | | | | | | | |
| VH | EVQLVESGGGLV | 633 | GFDF | 634 | ISGL | 599 | ATLGEY | 636 |
| H78 | QPGGSLRLSCAAS | | EDYG | | GGAT | | GDYDY | |
| | GFDFEDYGMGWF | | | | | | | |
| | RQAPGKGRELVA | | | | | | | |
| | AISGLGGATYYPD | | | | | | | |
| | SVEGRFTISRDNA | | | | | | | |
| | KRMVYLQMNSLR | | | | | | | |
| | AEDTAVYYCATL | | | | | | | |
| | GEYGDYDYWGQ | | | | | | | |
| | GTLVTVSS | | | | | | | |
| VH | EVQLVESGGGLV | 637 | GLNF | 638 | ISNK | 555 | ATLEWE | 640 |
| H79 | QPGGSLRLSCAAS | | GDY | | GGAT | | LDY | |
| | GLNFGDYAMGWF | | A | | | | | |
| | RQAPGKGRELVA | | | | | | | |
| | AISNKGGATYYPD | | | | | | | |
| | SVEGRFTISRDNA | | | | | | | |
| | KRMVYLQMNSLR | | | | | | | |
| | AEDTAVYYCATL | | | | | | | |
| | EWELDYWGQGTL | | | | | | | |
| | VTVSS | | | | | | | |
| VH | EVQLVESGGGLV | 641 | GFKF | 642 | ISGP | 567 | AGGAG | 644 |
| H80 | QPGGSLRLSCAAS | | GDY | | GGAT | | WLTDY | |
| | GFKFGDYAMGWF | | A | | | | | |
| | RQAPGKGRELVA | | | | | | | |
| | AISGPGGATYYPD | | | | | | | |
| | SVEGRFTISRDNA | | | | | | | |
| | KRMVYLQMNSLR | | | | | | | |
| | AEDTAVYYCAGG | | | | | | | |
| | AGWLTDYWGQG | | | | | | | |
| | TLVTVSS | | | | | | | |
| VH | DVQLVESGGGLV | 645 | GISLS | 646 | ISGSS | 511 | ASLGFT | 648 |
| H81 | QPGGSLRLSCAAS | | DYS | | ITT | | MIVGGI | |
| | GISLSDYSMGWFR | | | | | | DY | |
| | QAPGKEREFVAGI | | | | | | | |
| | SGSSITTYYADSV | | | | | | | |
| | KGRFTISRDNSKN | | | | | | | |
| | TVYLQMNSLRPE | | | | | | | |
| | DTAVYYCASLGF | | | | | | | |
| | TMIVGGIDYWGQ | | | | | | | |
| | GTLVTVSS | | | | | | | |
| VH | EVQLVESGGGLV | 649 | GFDF | 650 | ISAP | 515 | VYLGDA | 652 |
| H82 | QPGGSLRLSCAAS | | SGFS | | VGVT | | FDI | |
| | GFDFSGFSMGWF | | | | | | | |
| | RQAPGKGRELVA | | | | | | | |
| | AISAPVGVTYYPD | | | | | | | |
| | SVEGRFTISRDNA | | | | | | | |
| | KRMVYLQMNSLR | | | | | | | |
| | AEDTAVYYCVYL | | | | | | | |
| | GDAFDIWGQGTM | | | | | | | |
| | VTVSS | | | | | | | |
| VH | DVQLVESGGGLV | 653 | GFTIS | 654 | IMGS | 547 | AKQPPD | 656 |
| H83 | QPGGSLRLSCAAS | | GND | | GGTT | | WYFDL | |
| | GFTISGNDMGWF | | | | | | | |
| | RQAPGKEREFVA | | | | | | | |
| | AIMGSGGTTYYA | | | | | | | |
| | DSVKGRFTISRDN | | | | | | | |
| | SKNTVYLQMNSL | | | | | | | |
| | RPEDTAVYYCAK | | | | | | | |
| | QPPDWYFDLWGR | | | | | | | |
| | GTLVTVSS | | | | | | | |
| VH | EVQLVESGGGLV | 657 | GFAF | 658 | INAA | 659 | ARDEQIS | 660 |
| H84 | QPGGSLRLSCAAS | | SDTW | | GSDT | | GYAWY | |
| | GFAFSDTWMGWL | | | | | | FDL | |
| | RQAPGKEREFVA | | | | | | | |
| | AINAAGSDTYYA | | | | | | | |
| | DSVKGRFTISRDN | | | | | | | |
| | SKNTLYLQMNSL | | | | | | | |
| | RPEDTAVYYCAR | | | | | | | |
| | DEQISGYAWYFD | | | | | | | |
| | LWGRGTLVTVSS | | | | | | | |
| VH | EVQLVESGGGLV | 661 | GLAL | 662 | ISAG | 663 | ARNFEIR | 664 |
| H85 | QPGGSLRLSCAAS | | SSYD | | GEHT | | YFNP | |
| | GLALSSYDMAWY | | | | | | | |
| | RQAPGKGRELVA | | | | | | | |
| | GISAGGEHTSYAD | | | | | | | |
| | SVKGRFTISRDNA | | | | | | | |
| | KNTLYLQMNSLR | | | | | | | |
| | PEDTAVYYCARN | | | | | | | |
| | FEIRYFNPWGQGT | | | | | | | |
| | LVTVSS | | | | | | | |
| VH | EVQLVESGGGLV | 665 | GFNF | 421 | ISAP | 591 | ARVADY | 668 |
| H86 | QPGGSLRLSCAAS | | EEYG | | GGVT | | GSGSYS | |
| | GFNFEEYGMGWF | | | | | | SNWFDP | |
| | RQAPGKGRELVA | | | | | | | |
| | AISAPGGVTYYPD | | | | | | | |
| | SVEGRFTISRDNA | | | | | | | |
| | KRMVYLQMNSLK | | | | | | | |
| | SEDTAVYYCARV | | | | | | | |
| | ADYGSGSYSSNW | | | | | | | |
| | FDPWGQGTLVTV | | | | | | | |
| | SS | | | | | | | |
| VH | EVQLVESGGGLV | 669 | GLVF | 670 | ISAP | 591 | ARVCSE | 672 |
| H87 | QPGGSLRLSCAAS | | SDYS | | GGVT | | YCGGD | |
| | GLVFSDYSMGWF | | | | | | WLSAGY | |
| | RQAPGKGRELVA | | | | | | WYFDL | |
| | AISAPGGVTYYPD | | | | | | | |
| | SVEGRFTISRDNA | | | | | | | |
| | KRMVYLQMNSLR | | | | | | | |
| | AEDTAVYYCARV | | | | | | | |
| | CSEYCGGDWLSA | | | | | | | |
| | GYWYFDLWGRG | | | | | | | |
| | TLVTVSS | | | | | | | |
| VH | EVQLVESGGGLV | 673 | GFSV | 674 | ISGS | 675 | TRVVSG | 676 |
| H88 | QPGGSLRLSCAAS | | SDYD | | ASHT | | QQLVFP | |
| | GFSVSDYDMAWY | | | | | | LDY | |
| | RQAPGKGRELVA | | | | | | | |
| | GISGSASHTSYAD | | | | | | | |
| | SVKGRFTISRDNA | | | | | | | |
| | KNTLYLQMNSLR | | | | | | | |
| | PEDTAVYYCTRV | | | | | | | |
| | VSGQQLVFPLDY | | | | | | | |
| | WGQGSLVTVSS | | | | | | | |
| VH | EVQLVESGGGLV | 677 | GFDF | 678 | ISSH | 679 | ASLGSD | 680 |
| H89 | QPGGSLRLSCAAS | | GGY | | GGAT | | YGEGDD | |
| | GFDFGGYGMGWF | | G | | | | Y | |
| | RQAPGKGRELVA | | | | | | | |
| | AISSHGGATYYPD | | | | | | | |
| | SVEGRFTISRDNA | | | | | | | |
| | KRMVYLQMNSLR | | | | | | | |
| | AEDTAVYYCASL | | | | | | | |
| | GSDYGEGDDYWG | | | | | | | |
| | QGTLVTVSS | | | | | | | |
| VH | EVQLVESGGGLV | 681 | GITL | 682 | ISGL | 599 | VGGSG | 684 |
| H90 | QPGGSLRLSCAAS | | EDYA | | GGAT | | WLSDY | |
| | GITLEDYAMGWF | | | | | | | |
| | RQAPGKGRELVA | | | | | | | |
| | AISGLGGATYYPD | | | | | | | |
| | SVEGRFTISRDNA | | | | | | | |
| | KRMVYLQMNSLR | | | | | | | |
| | AEDTAVYYCVGG | | | | | | | |
| | SGWLSDYWGHGT | | | | | | | |
| | LVTVSS | | | | | | | |
| VH | DVQLVESGGGLV | 685 | GFPV | 686 | IMGS | 547 | ASQPED | 688 |
| H91 | QPGGSLRLSCAAS | | SNYD | | GGTT | | WYFDL | |
| | GFPVSNYDMGWF | | | | | | | |
| | RQAPGKEREFVA | | | | | | | |
| | AIMGSGGTTYYA | | | | | | | |
| | DSVKGRFTISRDN | | | | | | | |
| | SKNTVYLQMNSL | | | | | | | |
| | RPEDTAVYYCAS | | | | | | | |
| | QPEDWYFDLWGR | | | | | | | |
| | GTLVTVSS | | | | | | | |
| VH | EVQLVESGGGLV | 689 | GFNF | 690 | ISSGS | 691 | ARLPVV | 692 |
| H92 | QPGGSLRLSCAAS | | GDH | | GAT | | VTGDGA | |
| | GFNFGDHAMGWF | | A | | | | FDI | |
| | RQAPGKGRELVA | | | | | | | |
| | AISSGSGATYYPD | | | | | | | |
| | SVEGRFTISRDNA | | | | | | | |
| | KRMVYLQMNSLK | | | | | | | |
| | SEDTAVYYCARLP | | | | | | | |
| | VVVTGDGAFDIW | | | | | | | |
| | GQGTMVTVSS | | | | | | | |
| VH | DVQLVESGGGLV | 693 | GLPF | 510 | ISGSS | 511 | ATSGYS | 512 |
| H178 | QPGGSLRLSCAAS | | SDYS | | ITT | | YSAGGM | |
| | GLPFSDYSMGWF | | | | | | DV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYSAGGMDVWG | | | | | | | |
| | QGTTVTVSSPP | | | | | | | |
| VH | DVQLVESGGGLV | 694 | GLPF | 510 | ISGSS | 511 | ATSGYS | 512 |
| H179 | QPGGSLRLSCAAS | | SDYS | | ITT | | YVAGG | |
| | GLPFSDYSMGWF | | | | | | MDV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYVAGGMDVWG | | | | | | | |
| | QGTTVTVSSPP | | | | | | | |
| VH | DVQLVESGGGEV | 695 | GLPF | 510 | ISGSS | 511 | ATSGYS | 512 |
| H180 | QPGGSLRLSCAAS | | SDYS | | ITT | | YVAGG | |
| | GLPFSDYSMGWF | | | | | | MDV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYVAGGMDVWG | | | | | | | |
| | QGTTVTVSS | | | | | | | |
| VH | DVQLVESGGGEV | 696 | GLPF | 510 | ISGSS | 511 | ATSGYS | 512 |
| H181 | QPGGSLRLSCAAS | | SDYS | | ITT | | YVAGG | |
| | GLPFSDYSMGWF | | | | | | MDV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYVAGGMDVWG | | | | | | | |
| | QGTTVTVSSPP | | | | | | | |
| VH | DVQLVESGGGEV | 697 | GLPF | 510 | ISGSS | 699 | ATSGYS | 512 |
| H182 | QPGGSLRLSCAAS | | SDYS | | STT | | YVAGG | |
| | GLPFSDYSMGWF | | | | | | MDV | |
| | RQAPGKEREFVA | | | | | | | |
| | GISGSSSTTYYAD | | | | | | | |
| | SVKGRFTISRDNS | | | | | | | |
| | KNTLYLQMNSLR | | | | | | | |
| | PEDTAVYYCATS | | | | | | | |
| | GYSYVAGGMDV | | | | | | | |
| | WGQGTTVTVSSPP | | | | | | | |
| VH | DVQLVESGGGEV | 698 | GLPF | 510 | ISGSS | 511 | ATSGYS | 512 |
| H183 | QPGGSLRLSCAAS | | SDYS | | ITT | | YSAGGM | |
| | GLPFSDYSMGWF | | | | | | DV | |
| | RQAPGKEREFVA | | | | | | | |
| | GISGSSITTYYADS | | | | | | | |
| | VKGRFTISRDNSK | | | | | | | |
| | NTLYLQMNSLRPE | | | | | | | |
| | DTAVYYCATSGY | | | | | | | |
| | SYSAGGMDVWG | | | | | | | |
| | QGTTVTVSSPP | | | | | | | |
| VH | EVQLLESGGGLV | 175 | | | | | | |
| H103 | QPGGSLRLSCAAS | | | | | | | |
| | GSTFSSYTVSWFR | | | | | | | |
| | QAPGKEREFVSAII | | | | | | | |
| | GSSGHTYYSDSVK | | | | | | | |
| | GRFTISRDNSKNT | | | | | | | |
| | LYLQMNSLKPED | | | | | | | |
| | TAVYYCAADRVY | | | | | | | |
| | DHEEYGITEDYAY | | | | | | | |
| | GQGTLVTVSS | | | | | | | |
| VH | EVQLLESGGGLV | 176 | | | | | | |
| H104 | QPGGSLRLSCAAS | | | | | | | |
| | GFTFSSYFIGWFR | | | | | | | |
| | QAPGKEREFVSAII | | | | | | | |
| | GSGGSTYYDDAV | | | | | | | |
| | KGRFTISRDNSKN | | | | | | | |
| | TLYLQMNSLKPE | | | | | | | |
| | DTAVYYCAVKRH | | | | | | | |
| | AYYGRDRSYNYD | | | | | | | |
| | YWGQGTLVTVSS | | | | | | | |
| VH | EVQLLESGGGLV | 177 | | | | | | |
| H105 | QPGGSLRLSCAAS | | | | | | | |
| | GGTFSIYGIGWFR | | | | | | | |
| | QAPGKEREFVSGII | | | | | | | |
| | LTGGSTYYADSV | | | | | | | |
| | KGRFTISRDNSKN | | | | | | | |
| | TLYLQMNSLKPE | | | | | | | |
| | DTAVYYCYVDVP | | | | | | | |
| | LYDTGYHTYWGQ | | | | | | | |
| | GTLVTVSS | | | | | | | |
| VH | EVQLLESGGGLV | 178 | | | | | | |
| H106 | QPGGSLRLSCAAS | | | | | | | |
| | GSSFSSYDLGWFR | | | | | | | |
| | QAPGKEREFVSAY | | | | | | | |
| | ILSGGSTYYEDSV | | | | | | | |
| | KGRFTISRDNSKN | | | | | | | |
| | TLYLQMNSLKPE | | | | | | | |
| | DTAVYYCYADYT | | | | | | | |
| | VERDRYSYWGQG | | | | | | | |
| | TLVTVSS | | | | | | | |
| VH | EVQLLESGGGLV | 179 | | | | | | |
| H107 | QPGGSLRLSCAAS | | | | | | | |
| | GFIFSSYHLAWFR | | | | | | | |
| | QAPGKEREFVSAS | | | | | | | |
| | IGSSEDTEYADSV | | | | | | | |
| | KGRFTISRDNSKN | | | | | | | |
| | TLYLQMNSLKPE | | | | | | | |
| | DTAVYYCYADHG | | | | | | | |
| | VKYYEYWGRGTL | | | | | | | |
| | VTVSS | | | | | | | |
| VH | EVQLLESGGGLV | 180 | | | | | | |
| H108 | QPGGSLRLSCAAS | | | | | | | |
| | GRTYEIYGMGWF | | | | | | | |
| | RQAPGKEREFVSG | | | | | | | |
| | SISSEGSTFYADSV | | | | | | | |
| | KGRFTISRDNSKN | | | | | | | |
| | TLYLQMNSLKPE | | | | | | | |
| | DTAVYYCYADQS | | | | | | | |
| | YPYGYTTYWGQG | | | | | | | |
| | TLVTVSS | | | | | | | |
| VH | EVQLLESGGGLV | 181 | | | | | | |
| H109 | QPGGSLRLSCAAS | | | | | | | |
| | GSSFSIYDMGWFR | | | | | | | |
| | QAPGKEREFVSAY | | | | | | | |
| | ITSGGDTYYEDSV | | | | | | | |
| | KGRFTISRDNSKN | | | | | | | |
| | TLYLQMNSLKPE | | | | | | | |
| | DTAVYYCYAGVP | | | | | | | |
| | QDEDYWGQGTLV | | | | | | | |
| | TVSS | | | | | | | |
| VH | EVQLLESGGGLV | 182 | | | | | | |
| H110 | QPGGSLRLSCAAS | | | | | | | |
| | GRIFSILDVGWFR | | | | | | | |
| | QAPGKEREFVSAI | | | | | | | |
| | SGSSGGRYYADS | | | | | | | |
| | VKGRFTISRDNSK | | | | | | | |
| | NTLYLQMNSLKP | | | | | | | |
| | EDTAVYYCSVLES | | | | | | | |
| | EYQLYDYWGQGT | | | | | | | |
| | LVTVSS | | | | | | | |
| VH | EVQLLESGGGLV | 183 | | | | | | |
| H111 | QPGGSLRLSCAAS | | | | | | | |
| | GRTLSSYDMGWF | | | | | | | |
| | RQAPGKEREFVSA | | | | | | | |
| | FITSGGSTFYEDSV | | | | | | | |
| | KGRFTISRDNSKN | | | | | | | |
| | TLYLQMNSLKPE | | | | | | | |
| | DTAVYYCYADYV | | | | | | | |
| | HDRGYWGQGTLV | | | | | | | |
| | TVSS | | | | | | | |
| VH | EVQLLESGGGLV | 184 | | | | | | |
| H112 | QPGGSLRLSCAAS | | | | | | | |
| | GSTFSSYTMAWF | | | | | | | |
| | RQAPGKEREFVSA | | | | | | | |
| | NITSGGDTYYADS | | | | | | | |
| | VKGRFTISRDNSK | | | | | | | |
| | NTLYLQMNSLKP | | | | | | | |
| | EDTAVYYCAADV | | | | | | | |
| | VYDEDYWGLSTH | | | | | | | |
| | YTVSS | | | | | | | |
| VH | EVQLLESGGGLV | 185 | | | | | | |
| H113 | QPGGSLRLSCAAS | | | | | | | |
| | GFEFSIYDLGWFR | | | | | | | |
| | QAPGEEREFVSAY | | | | | | | |
| | ITSEGSTYYVDSV | | | | | | | |
| | KGRFTISRDNSKN | | | | | | | |
| | TLYLQMNSLKPE | | | | | | | |
| | DTAVYYCYADRG | | | | | | | |
| | LHYSYWGQGTLV | | | | | | | |
| | TVSS | | | | | | | |
| VH | EVQLLESGGGLV | 186 | | | | | | |
| H114 | QPGGSLRLSCAAS | | | | | | | |
| | GIIFSIYDMGWFR | | | | | | | |
| | QAPGKEREFVSAF | | | | | | | |
| | ITSGGSTYYPDSV | | | | | | | |
| | KGRFTISRDNSKN | | | | | | | |
| | TLYLQMNSLKPE | | | | | | | |
| | DTAVYYCYAEEG | | | | | | | |
| | VGLEGYWGQGTL | | | | | | | |
| | VTVSS | | | | | | | |
| VH | EVQLLESGGGLV | 187 | | | | | | |
| H115 | QPGGSLRLSCAAS | | | | | | | |
| | GSPEREYGVGWF | | | | | | | |
| | RQAPGKEREFVSG | | | | | | | |
| | VISSGGSTYYADS | | | | | | | |
| | VKGRFTISRDNSK | | | | | | | |
| | NTLYLQMNSLKP | | | | | | | |
| | EDTAVYYCYADH | | | | | | | |
| | GTDQGSYTYWGQ | | | | | | | |
| | GTLVTVSS | | | | | | | |
| VH | EVQLLESGGGLV | 188 | | | | | | |
| H116 | QPGGSLRLSCAAS | | | | | | | |
| | GSFFSSYSVGWFR | | | | | | | |
| | QAPGKEREFVSAI | | | | | | | |
| | VIRTGSTYYGDSV | | | | | | | |
| | KGRFTISRDNSKN | | | | | | | |
| | TLYLQMNSLKPE | | | | | | | |
| | DTAVYYCYADES | | | | | | | |
| | FDQESNWGQGTL | | | | | | | |
| | VTVSS | | | | | | | |
| VH | EVQLLESGGGLV | 189 | | | | | | |
| H117 | QPGGSLRLSCAAS | | | | | | | |
| | GSSFSSYIMGWFR | | | | | | | |
| | QAPGKEREFVSAV | | | | | | | |
| | IGSGGDTYYADSV | | | | | | | |
| | KGRFTISRDNSKN | | | | | | | |
| | TLYLQMNSLKPE | | | | | | | |
| | DTAVYYCYVDAD | | | | | | | |
| | TPVTTSHWGQGT | | | | | | | |
| | LVTVSS | | | | | | | |
| VH | EVQLLESGGGLV | 190 | | | | | | |
| H118 | QPGGSLRLSCAAS | | | | | | | |
| | GSIFSSYAMGWFR | | | | | | | |
| | QAPGKEREFVSAI | | | | | | | |
| | VGSSGSTYYADSV | | | | | | | |
| | KGRFTISRDNSKN | | | | | | | |
| | TLYLQMNSLKPE | | | | | | | |
| | DTAVYYCAVQRG | | | | | | | |
| | IDYSFDFWGQGTL | | | | | | | |
| | VTVSS | | | | | | | |
| VH | EVQLLESGGGLV | 191 | | | | | | |
| H119 | QPGGSLRLSCAAS | | | | | | | |
| | GSIFSIYAMGWFR | | | | | | | |
| | QAPGKEREFVSAS | | | | | | | |
| | VISSGSTYYADSV | | | | | | | |
| | KGRFTISRDNSKN | | | | | | | |
| | TLYLQMNSLKPE | | | | | | | |
| | DTAVYYCSAHTD | | | | | | | |
| | YSLYDYWGQGTL | | | | | | | |
| | VTVSS | | | | | | | |
| VH | EVQLLESGGGLV | 192 | | | | | | |
| H120 | QPGGSLRLSCAAS | | | | | | | |
| | GFIFSFYHLAWFR | | | | | | | |
| | QAPGKEREFVSAS | | | | | | | |
| | IGSSEDTEYADSV | | | | | | | |
| | KGRFTISRDNSKN | | | | | | | |
| | TLYLQMNSLKPE | | | | | | | |
| | DTAVYYCYAVHG | | | | | | | |
| | VKYYEYWGQGTL | | | | | | | |
| | VTVSS | | | | | | | |
| VH | EVQLLESGGGLV | 193 | | | | | | |
| H121 | QPGGSLRLSCAAS | | | | | | | |
| | GSIFEFYAVGWFR | | | | | | | |
| | QAPGKEREFVSAT | | | | | | | |
| | ISSTGSKYYEDSV | | | | | | | |
| | KGRFTISRDNSKN | | | | | | | |
| | TLYLQMNSLKPE | | | | | | | |
| | DTAVYYCAVDIG | | | | | | | |
| | EGFDYWGQGTLV | | | | | | | |
| | TVSS | | | | | | | |
| VH | EVQLLESGGGLV | 194 | | | | | | |
| H122 | QPGGSLRLSCAAS | | | | | | | |
| | GSIFSYYGMGWF | | | | | | | |
| | RQAPGKEREFVSG | | | | | | | |
| | IISSGGSTYYADSV | | | | | | | |
| | KGRFTISRDNSKN | | | | | | | |
| | TLYLQMNSLKPE | | | | | | | |
| | DTAVYYCYADTY | | | | | | | |
| | TQRYTYWGQGTL | | | | | | | |
| | VTVSS | | | | | | | |
| VH | EVQLLESGGGLV | 195 | | | | | | |
| H123 | QPGGSLRLSCAAS | | | | | | | |
| | GSIFSSYAVGWFR | | | | | | | |
| | QAPGKEREFVSAQ | | | | | | | |
| | IISSGSTYYEDSVK | | | | | | | |
| | GRFTISRDNSKNT | | | | | | | |
| | LYLQMNSLKPED | | | | | | | |
| | TAVYYCAAEEYY | | | | | | | |
| | GYFDYWGQGTLV | | | | | | | |
| | TVSS | | | | | | | |
| VH | EVQLLESGGGLV | 196 | | | | | | |
| H124 | QPGGSLRLSCAAS | | | | | | | |
| | GSIFSIYAMSWFR | | | | | | | |
| | QAPGKEREFVSAL | | | | | | | |
| | IGSGGSTFYADSV | | | | | | | |
| | KGRFTISRDNSKN | | | | | | | |
| | TLYLQMNSLKPE | | | | | | | |
| | DTAVYYCYATVG | | | | | | | |
| | DHPQFQYDYWGQ | | | | | | | |
| | GTLVTVSS | | | | | | | |
| VH | EVQLLESGGGLV | 197 | | | | | | |
| H125 | QPGGSLRLSCAAS | | | | | | | |
| | GRIFSRYAMGWF | | | | | | | |
| | RQAPGKEREFVSA | | | | | | | |
| | YIATSGSRYYGDS | | | | | | | |
| | VKGRFTISRDNSK | | | | | | | |
| | NTLYLQMNSLKP | | | | | | | |
| | EDTAVYYCGVSL | | | | | | | |
| | PQGTGQGYDLYD | | | | | | | |
| | TWGQGTLVTVSS | | | | | | | |
| VH | EVQLLESGGGLV | 198 | | | | | | |
| H126 | QPGGSLRLSCAAS | | | | | | | |
| | GFEFSIYHLGWFR | | | | | | | |
| | QAPGEEREFVSAY | | | | | | | |
| | ITSEGSTYYVDSV | | | | | | | |
| | KGRFTISRDNSKN | | | | | | | |
| | TLYLQMNSLKPE | | | | | | | |
| | DTAVYYCYADRG | | | | | | | |
| | LHYSYWGQGTLV | | | | | | | |
| | TVSS | | | | | | | |

In some embodiments, an anti-PD-1 VHH of the immunocytokine composition comprises one or more modifications which imparts the VHH with reduced immunogenicity or reduced binding of pre-existing antibodies to the VHH. Examples of such modifications are described in, for example, U.S. Patent Publication Nos. US20180009888A9 (e.g., extension peptides of 1 to 5 extending beyond the C-terminal “SS” of the VHH, such as those consisting of the amino acids Ala and Gly), US20160207981A1 (e.g., substitutions of the C-terminal “SS” of the VHH, such as with an amino acid or peptide of a sequence E, SE, EG, SEG, EP, EPG, DP, DPG, K, SK, KP, KPG, RP, or RPG and/or substitutions of Leu 11 of the VHH, such as an L11K, L11R, L11D, or LiHE substitution), US20140161796A1 (e.g., deletions of certain sequences from the VHH), US20170121399A1 (e.g., substitutions of Leu 11 of the VHH (e.g., L11K or L11V) and/or Leu 89 (e.g., L89T)), and Lin et al., “A structure-based engineering approach to abrogate pre-existing antibody binding to biotherapeutics,” PLoS ONE 16(7): e0254944. doi.org/10.1371/journal.pone.0254944 (e.g., the addition of 1, 2, or 3 prolines beyond the C-terminal “SS” of the VHH, such as a two-proline peptide). In some embodiments, the anti-PD-1 VHH (e.g., any of those described in the tables above) comprises a C-terminal modification of the addition of 1, 2 or 3 prolines to the C-terminus of the VHH (e.g., to the C-terminus of any of the VHHs described herein). In some embodiments, the anti-PD-1 VHH comprises a C-terminal modification of the addition of 2 prolines to the C-terminus of the VHH. In embodiments where two anti-PD-1 VHHs are linked in series (e.g., by a flexible peptide linker), in some instance only the C-terminal VHH will comprise the modification (e.g., only the C-terminal VHH comprises the sequence “PP” after the C-terminal “SS” of the VHH). In some instances, both VHHs linked in series will contain the modification (e.g., both will comprise the sequence “PP” after the C-terminal “SS” of each VHH).

In some embodiments, the anti-PD-1 binding domain of the immunocytokine composition comprises a light chain constant region (e.g., in cases in which the anti-PD-1 binding domain is a Fab). In some embodiments, the light chain constant region is one which contains one or more modifications which enhance the stability and/or manufacturability of the binding domain (or the immunocytokine composition as a whole). Such modifications are described in, for example, U.S. Pat. No. 9,777,067 and U.S. Patent Publication No. US20150239977A1 and include, for example, modifications of residue L154 of the light chain constant region (EU numbering), such as an L154K substitution and/or modifications of residue L201 (EU numbering), such as an L201K substitution. In some embodiments, the light chain constant region of the anti-PD-1 binding domain (e.g., a Fab) comprises an L154K substitution compared to the consensus sequence (e.g., as in SEQ ID NO: 277). In some embodiments, the light chain constant region of the anti-PD-1 binding domain (e.g., a Fab) comprises an L201K substitution compared to the consensus sequence. In some embodiments, the light chain constant region of the anti-PD-1 binding domain (e.g., a Fab) comprises L154K and L201K substitutions compared to the consensus sequence (e.g., as in SEQ ID NO: 278).

### VEGFA Targeting Binding Domains

In some embodiments, an immunocytokine composition of the instant disclosure comprise one or more binding domains which target vascular endothelial growth factor A (VEGFA). In some embodiments, a binding domain incorporated into an immunocytokine composition of the disclosure specifically binds to VEGFA. In some embodiments, the anti-VEGFA binding domain is capable of disrupting and/or preventing the interaction of VEGFA with one or more of its receptors (e.g., VEGFR1, VEGFR2, and/or VEGFR3). In some embodiments, the anti-VEGFA binding domain is capable of disrupting and/or preventing the interaction of VEGFA with VEGFR1. In some embodiments, the anti-VEGFA binding domain is capable of disrupting and/or preventing the interaction of VEGFA with VEGFR2. In some embodiments, the anti-VEGFA binding domain is capable of disrupting and/or preventing the interaction of VEGFA with VEGFR3. In some embodiments, the anti-VEGFA binding domain is capable of disrupting and/or preventing the interaction of VEGFA with each of VEGFR1, VEGFR2, and VEGFR3.

A non-limiting, exemplary, human VEGFA amino acid sequence is LTDRQTDTAPSPSYHLLPGRRRTVDAAASRGQGPEPAPGGGVEGVGARGVALKLFVQL LGCSRFGGAVVRAGEAEPSGAARSASSGREEPQPEEGEEEEEKEEERGPQWRLGARKPG SWTGEAAVCADSAPAARAPQALARASGRGGRVARRGAEESGPPHSPSRRGSASRAGPG RASETMNFLLSWVHWSLALLLYLHHAKWSQAAPMAEGGGQNHHEVVKFMDVYQRSY CHPIETLVDIFQEYPDEIEYIFKPSCVPLMRCGGCCNDEGLECVPTEESNITMQIMRIKPHQ GQHIGEMSFLQHNKCECRCDKPRR (SEQ ID NO: 120) (UniProt ID A0A0A0MR43). VEGFA is also referred to VEGF. The terms “VEGFA” and “VEGF” are used interchangeably herein.

In some embodiments, the anti-VEGFA binding domain is comprised in an antigen binding fragment derived from an antibody. For example, the anti-VEGFA binding domain can be derived from any anti-VEGFA antibody known in the art or which can be made according to well understood methods. In some embodiments, the binding domain of an immunocytokine composition described herein is derived from an anti-VEGFA antibody or antigen binding fragment.

In one embodiment, an anti-VEGFA binding domain of an immunocytokine composition comprises and antigen binding fragment. In some embodiments, an anti-VEGFA binding domain of the disclosure comprises a combination of a heavy chain variable region (VH) and a light chain variable region (VL) described herein, or those of an antibody or antigen binding fragment otherwise known in the art. In another embodiment, an anti-VEGFA binding domain of the disclosure comprises a combination of complementarity determining regions (VH CDR1, VH CDR2, VH CDR3, VL CDR1, VL CDR2, and VL CDR3) described herein, or those of an antibody or antigen binding fragment otherwise known in the art.

In one embodiment, an anti-VEGFA binding domain of the disclosure comprises the CDRs of an antibody selected from Bevacizumab, Brolucizumab, Faricimab, Ranibizumab, Ivonescimab, AI-081, HLX-04, or IBI305 incorporated into a VH and VL. In some embodiments, the anti-VEGFA binding domain comprises the VH and VL of any one of these antibodies. In some embodiments, the anti-VEGFA binding domain comprises the VH and VL of any one of these antibodies or the VH and VL which include the CDRs of these antibodies in one of the following formats: a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a Fab-Fc, a scFv-Fc, or a bispecific antibody. In some embodiments, the VH and VL of any one of these antibodies or the VH and VL which include the CDRs of these antibodies is comprised in a Fab, a Fab′, or an scFv. In some embodiments, the VH and VL any one of these antibodies or of the VH and VL which include the CDRs of these antibodies is comprised in a Fab or an scFv. In some embodiments, the VH and VL of any of these antibodies or the VH and VL which include the CDRs of these antibodies is comprised in a Fab. In some embodiments, the VH and VL of any of these antibodies or the VH and VL which include the CDRs of these antibodies is comprised in an scFv.

In some embodiments, an anti-VEGFA binding domain of the disclosure comprises the CDRs (e.g., VH and VL CDRs) of Bevacizumab, Brolucizumab, Faricimab, Ranibizumab, Ivonescimab, AI-081, HLX-04, or IBI305 incorporated into a VH and VL. In one embodiment, an anti-PD-1 binding domain of the disclosure comprises the VH and VL of Bevacizumab, Brolucizumab, Faricimab, Ranibizumab, Ivonescimab, AI-081, HLX-04, or IBI305. In some embodiments, the anti-VEGFA binding domain comprises the VH and VL of any one of these antibodies or the VH and VL which include the CDRs of these antibodies in one of the following formats: a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a Fab-Fc, a scFv-Fc, or a bispecific antibody. In some embodiments, the VH and VL of any one of these antibodies or the VH and VL which include the CDRs of these antibodies is comprised in a Fab, a Fab′, or an scFv. In some embodiments, the VH and VL any one of these antibodies or of the VH and VL which include the CDRs of these antibodies is comprised in a Fab or an scFv. In some embodiments, the VH and VL of any of these antibodies or the VH and VL which include the CDRs of these antibodies is comprised in a Fab. In some embodiments, the VH and VL of any of these antibodies or the VH and VL which include the CDRs of these antibodies is comprised in an scFv.

In some embodiments, the anti-VEGFA binding domain comprises the VH and VL of Bevacizumab, Brolucizumab, Faricimab, or Ranibizumab. In some embodiment, the anti-VEGFA binding domain comprises the VH and VL of Bevacizumab or Brolucizumab. In some embodiments, the anti-VEGFA binding domain comprises the VH and VL of Bevacizumab. In some embodiments, the anti-VEGFA binding domain comprises the VH and VL of Brolucizumab. In some embodiments, the anti-VEGFA binding domain comprises the VH and VL of Bevacizumab in a Fab or scFv format.

TABLES 2A and 2B provide the sequences of exemplary anti-EGFA antibodies and anti-VEGFA antigen binding fragments which contain sets of CDRs which can be incorporated into VHs and/or VLs and used as anti-VEGFA binding domains as described herein. In some embodiments, the VHs and VLs of the antibodies in Table 2A or 2B are incorporated into anti-VEGFA binding domains (e.g., in a Fab or scFv format). In some embodiments, CDRs of a VH described in Table 2A or 2B is incorporated into a binding domain as a VHH. In some embodiments, a VH as described in Table 2A or 2B is incorporated into a binding domain as a VHH.

In some instances, the sequences listed in Table 2A or 2B contain full-length heavy or light chains of the indicated antibodies with the VH or VL respectively indicated in bold. Where there is a reference herein to a VH or VL of a sequence in Table 2A or 2B which contains a full-length heavy or light chain, it is intended to reference the bolded portion of the sequence.

An anti-VEGFA binding domain can comprise a VH having an amino acid sequence of any one of those described in Table 2A or 2B. The anti-VEGFA binding domain can comprise (or further comprise) a VL having an amino acid sequence of any one of those described in Table 2A or 2B. In preferred embodiments, the VH and VL are from the same antibody or antigen binding fragment described in Table 2A.

An anti-VEGFA binding domain can comprise a heavy chain, a VH, or a VH-CH1 domain (e.g., as in a Fab) having an amino acid sequence of any one of those set forth in Table 2A or 2B, or a portion corresponding to a VH thereof (e.g., the portion depicted in bold). An anti-VEGFA binding domain can comprise (or further comprise) a light chain (e.g., as in a Fab) or VL having an amino acid sequence of any one of those described in Table 2A or 2B, or a portion corresponding to a VL thereof. In preferred embodiments, the heavy chain, VH, or VH-CH1 domain and VL or light chain are from the same antibody or antigen binding fragment described in Table 2A or 2B.

In some embodiments, an anti-VEGFA binding domain comprises a VH having an amino acid sequence shown in Table 2A, and a VL having an amino acid sequence shown in Table 2A. In some embodiments, an anti-VEGFA binding domain comprises a VH and VL of Bevacizumab as shown in Table 2A. In some embodiments, an anti-VEGFA binding domain comprises a VH and VL of Brolucizumab as shown in Table 2A. In some embodiments, an anti-VEGFA binding domain comprises a VH and VL of Faricimab as shown in Table 2A. In some embodiments, an anti-VEGFA binding domain comprises a VH and VL of Ranibizumab as shown in Table 2A. In some embodiments, an anti-VEGFA binding domain comprises a VH and VL of Ivonescimab as shown in Table 2A. In some embodiments, an anti-VEGFA binding domain comprises a VH and VL of AI-081 as shown in Table 2A. In some embodiments, an anti-VEGFA binding domain comprises a VH and VL of HLX-04 as shown in Table 2A. In some embodiments, an anti-VEGFA binding domain comprises a VH and VL of IBI305 as shown in Table 2A.

In some embodiments, an anti-VEGFA binding domain comprises a VH CDR1 a VH CDR2 and a VH CDR3 of an antibody or antigen binding fragment as shown in Table 2A comprised in a VH and a VL CDR1, a VL CDR2, and a VL CDR3 of the same antibody or antigen binding fragment as shown in Table 2A comprised in a VL. In some embodiments, the anti-VEGFA binding domain comprises a corresponding VH and VL having at least 80%, 85%, 90%, 95%, 96%, 97%, 98%, 99%, or 100% identity to the VH and VL of the parent antibody (e.g., contains the indicated CDRs and the VH and VL each comprise the indicated sequence identity overall).

In some embodiments, an anti-VEGFA binding domain comprises a VH CDR1 a VH CDR2 and a VH CDR3 of Bevacizumab as shown in Table 2A comprising in a VH and a VL CDR1, a VL CDR2, and a VL CDR3 of Bevacizumab as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of Bevacizumab as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of Brolucizumab as shown in Table 2A comprised in a VH and a VL CDR1, a VL CDR2, and a VL CDR3 of Brolucizumab as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of Brolucizumab as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of Faricimab as shown in Table 2A comprised in a VH and a VL CDR1, a VL CDR2, and a VL CDR3 of Faricimab as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of Faricimab as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of Ranibizumab as shown in Table 2A comprised in a VH and a VL CDR1, a VL CDR2, and a VL CDR3 of Ranibizumab as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of Ranibizumab as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of Ivonescimab as shown in Table 2A comprised in a VH and a VL CDR1, a VL CDR2, and a VL CDR3 of Ivonescimab as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of Ivonescimab as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of AI-081 as shown in Table 2A comprised in a VH and a VL CDR1, a VL CDR2, and a VL CDR3 of AI-081 as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of AI-081 as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of HLX-04 as shown in Table 2A comprised in a VH and a VL CDR1, a VL CDR2, and a VL CDR3 of HLX-04 as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of HLX-04 as shown in Table 2A (e.g., contains the indicated CDRs and the VH and VL each comprise the indicated sequence identity overall).

In some embodiments, an anti-VEGFA binding domain comprises a VH CDR1 a VH CDR2 and a VH CDR3 of IBI305 as shown in Table 2A comprised in a VH and a VL CDR1, a VL CDR2, and a VL CDR3 of IB1I305 as shown in Table 2A comprised in a VL. In some embodiments, the anti-VEGFA binding domain comprises a VH and VL having at least 80%, 85%, 90%, 95%, 96%, 97%, 98%, 99%, or 100% identity to the VH and VL of IBI305 as shown in Table 2A (e.g., contains the indicated CDRs and the VH and VL each comprise the indicated sequence identity overall).

In some embodiments, the anti-VEGFA binding domain is a single domain antibody comprising a CDR1 as set forth in SEQ ID NO: 202, a CDR2 as set forth in SEQ ID NO: 203, and a CDR3 as set forth in SEQ ID NO: 204. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 201 (e.g., the anti-VEGFA binding domain comprises the indicated sequence identity to SEQ ID NO: 201 and retains the CDRs).

In some embodiments, the anti-VEGFA binding domain is a single domain antibody comprising a CDR1 as set forth in SEQ ID NO: 206, a CDR2 as set forth in SEQ ID NO: 207, and a CDR3 as set forth in SEQ ID NO: 208. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 205 (e.g., the anti-VEGFA binding domain comprises the indicated sequence identity to SEQ ID NO: 205 and retains the CDRs).

In some embodiments, the anti-VEGFA binding domain is a single domain antibody comprising a CDR1 as set forth in SEQ ID NO: 210, a CDR2 as set forth in SEQ ID NO: 211, and a CDR3 as set forth in SEQ ID NO: 212. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 209 (e.g., the anti-VEGFA binding domain comprises the indicated sequence identity to SEQ ID NO: 209 and retains the CDRs).

In some embodiments, the anti-VEGFA binding domain is a single domain antibody comprising a CDR1 as set forth in SEQ ID NO: 202, a CDR2 as set forth in SEQ ID NO: 215, and a CDR3 as set forth in SEQ ID NO: 216. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 213 (e.g., the anti-VEGFA binding domain comprises the indicated sequence identity to SEQ ID NO: 213 and retains the CDRs).

In some embodiments, the anti-VEGFA binding domain is a single domain antibody comprising a CDR1 as set forth in SEQ ID NO: 218, a CDR2 as set forth in SEQ ID NO: 219, and a CDR3 as set forth in SEQ ID NO: 220. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 200, 217, 221, 280, or 281 (e.g., the anti-VEGFA binding domain comprises the indicated sequence identity to SEQ ID NO: 200, 217, 221, 280, or 281 and retains the CDRs).

In some embodiments, the anti-VEGFA binding domain is a single domain antibody comprising a CDR1 as set forth in SEQ ID NO: 218, a CDR2 as set forth in SEQ ID NO: 279, and a CDR3 as set forth in SEQ ID NO: 220. In some embodiments, the CDRs are comprised in a VHH. In some embodiments, the anti-VEGFA binding domain comprises an amino acid sequence having 80%, 85%, 90%, 95%, 96%, 97%, 98%, 99%, or 100% to the sequence set forth in SEQ ID NO: 282-284 (e.g., the anti-VEGFA binding domain comprises the indicated sequence identity to SEQ ID NO: 282-284 and retains the CDRs).

In some embodiments, the anti-VEGFA binding domain is a single domain antibody comprising a CDR1 as set forth in SEQ ID NO: 223, a CDR2 as set forth in SEQ ID NO: 224, and a CDR3 as set forth in SEQ ID NO: 225. In some embodiments, the CDRs are comprised in a light chain single domain antibody. In some embodiments, the anti-VEGFA binding domain comprises an amino acid sequence having 80%, 85%, 90%, 95%, 96%, 97%, 98%, 99%, or 100% to the sequence set forth in SEQ ID NO: 222 (e.g., the anti-VEGFA binding domain comprises the indicated sequence identity to SEQ ID NO: 222 and retains the CDRs).

In some embodiments, a dual binding composition comprises one of the antibodies described in Table 2A below (optionally comprising one or more modifications to the Fc domain, hinge region, or other modification described herein) fused to a binding domain specific for PD-1 (e.g., any of the PD-1 binding domains described herein). In some embodiments, the binding domain specific for PD-1 is fused to the C-terminus of the heavy chain of the antibody described in Table 2A. In some embodiments, the binding domain specific for PD-1 fused to the antibody of Table 2A is an anti-PD-1 single-domain antibody as described herein.

In some embodiments, an immunocytokine composition comprises one or two anti-VEGFA binding domains. In some embodiments, the immunocytokine composition comprises one anti-VEGFA binding domain. In some embodiments, the immunocytokine composition comprises two anti-VEGFA binding domains. In some embodiments, each anti-VEGFA binding domain is a Fab or scFv. In some embodiments, each anti-VEGFA binding domain is a single domain antibody (e.g., a VHH). In some embodiments, the immunocytokine composition comprises two copies of the same anti-VEGFA binding domain. In some embodiments, the immunocytokine composition comprises two anti-VEGFA Fabs. In some embodiments, the immunocytokine composition comprises one anti-VEGFA Fab and one anti-VEGFA scFv. In some embodiments, the anti-VEGFA Fab and the anti-VEGFA scFv comprise the same VH and VL. In some embodiments, the immunocytokine composition comprises one anti-VEGFA Fab and one anti-VEGFA single domain antibody. In some embodiments, the immunocytokine composition comprises two anti-VEGFA single domain antibodies. In some embodiments, the immunocytokine composition comprises two anti-VEGFA VHHs.

TABLE 2A
Exemplary Antibodies Targeting VEGFA From Which anti-VEGFA Binding
Domains Can be Derived

| Antibody | |
|---|---|
| or Ag- | |
| binding | |
| fragment | Sequence |
| Bevacizumab | HC: |
| Heavy | EVQLVESGGGLVQPGGSLRLSCAASGYTFTNYGMNWVRQAPGKGLEWVG |
| Chain | WINTYTGEPTYAADFKRRFTFSLDTSKSTAYLQMNSLRAEDTAVYYCAKYP |
| | HYYGSSHWYFDVWGQGTLVTVSSASTKGPSVFPLAPSSKSTSGGTAALGCL |
| | VKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK (SEQ ID |
| | NO: 121) |
| | VH: |
| | EVQLVESGGGLVQPGGSLRLSCAASGYTFTNYGMNWVRQAPGKGLEWVG |
| | WINTYTGEPTYAADFKRRFTFSLDTSKSTAYLQMNSLRAEDTAVYYCAKYP |
| | HYYGSSHWYFDVWGQGTLVTVSS (SEQ ID NO: 122) |
| | VH CDR1: GYTFTNYGMN (SEQ ID NO: 123) |
| | VH CDR2: WINTYTGEPTYAADFK (SEQ ID NO: 124) |
| | VH CDR3: YPHYYGSSHWYFDV (SEQ ID NO: 125) |
| Bevacizumab | LC: |
| light | DIQMTQSPSSLSASVGDRVTITCSASQDISNYLNWYQQKPGKAPKVLIYFTSS |
| chain | LHSGVPSRFSGSGSGTDFTLTISSLQPEDFATYYCQQYSTVPWTFGQGTKVEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC (SEQ ID NO: 126) |
| | VL: |
| | DIQMTQSPSSLSASVGDRVTITCSASQDISNYLNWYQQKPGKAPKVLIYFTSS |
| | LHSGVPSRFSGSGSGTDFTLTISSLQPEDFATYYCQQYSTVPWTFGQGTKVEI |
| | K (SEQ ID NO: 127) |
| | VL CDR1: SASQDISNYLN (SEQ ID NO: 128) |
| | VL CDR2: FTSSLHS (SEQ ID NO: 129) |
| | VL CDR3: QQYSTVPWT (SEQ ID NO: 130) |
| Brolucizumab | VH: |
| VH | EVQLVESGGGLVQPGGSLRLSCTASGFSLTDYYYMTWVRQAPGKGLEWVG |
| | FIDPDDDPYYATWAKGRFTISRDNSKNTLYLQMNSLRAEDTAVYYCAGGD |
| | HNSGWGLDIWGQGTLVTVSS (SEQ ID NO: 132) |
| | VH CDR1: DYYYMT (SEQ ID NO: 133) |
| | VH CDR2: FIDPDDDPYYATWAKG (SEQ ID NO: 134) |
| | VH CDR3: GDHNSGWGLDI (SEQ ID NO: 135) |
| Brolucizumab | VL: |
| VL | EIVMTQSPSTLSASVGDRVIITCQASEIIHSWLAWYQQKPGKAPKLLIYLAST |
| | LASGVPSRFSGSGSGAEFTLTISSLQPDDFATYYCQNVYLASTNGANFGQGT |
| | KLTVLG (SEQ ID NO: 137) |
| | VL CDR1: QASEIIHSWLA (SEQ ID NO: 138) |
| | VL CDR2: LASTLAS (SEQ ID NO: 139) |
| | VL CDR3: QNVYLASTNGAN (SEQ ID NO: 140) |
| Faricimab | HC: |
| heavy | EVQLVESGGGLVQPGGSLRLSCAASGYDFTHYGMNWVRQAPGKGLEW |
| chain | VGWINTYTGEPTYAADFKRRFTFSLDTSKSTAYLQMNSLRAEDTAVYY |
| | CAKYPYYYGTSHWYFDVWGQGTLVTVSSASTKGPSVFPLAPSSKSTSGGT |
| | AALGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPEAAGGPSVFLF |
| | PPKPKDTLMASRTPEVTCVVVDVSHEDPEVKFNWYVDGVEVHNAKTKPRE |
| | EQYNSTYRVVSVLTVLAQDWLNGKEYKCKVSNKALGAPIEKTISKAKGQPR |
| | EPQVYTLPPCRDELTKNQVSLWCLVKGFYPSDIAVEWESNGQPENNYKTTP |
| | PVLDSDGSFFLYSKLTVDKSRWQQGNVFSCSVMHEALHNAYTQKSLSLSP |
| | (SEQ ID NO: 141) |
| | VH: |
| | EVQLVESGGGLVQPGGSLRLSCAASGYDFTHYGMNWVRQAPGKGLEWVG |
| | WINTYTGEPTYAADFKRRFTFSLDTSKSTAYLQMNSLRAEDTAVYYCAKYP |
| | YYYGTSHWYFDVWGQGTLVTV (SEQ ID NO: 142) |
| | VH CDR1: HYGMN (SEQ ID NO: 143) |
| | VH CDR2: WINTYTGEPTYAADFK (SEQ ID NO: 124) |
| | VH CDR3: YPYYYGTSHWYFDV (SEQ ID NO: 125) |
| Faricimab | LC: |
| light | DIQLTQSPSSLSASVGDRVTITCSASQDISNYLNWYQQKPGKAPKVLIYF |
| chain | TSSLHSGVPSRFSGSGSGTDFTLTISSLQPEDFATYYCQQYSTVPWTFGQ |
| | GTKVEIKRTVAAPSVFIFPPSDEQLKSGTASVVCLLNNFYPREAKVQWKVD |
| | NALQSGNSQESVTEQDSKDSTYSLSSTLTLSKADYEKHKVYACEVTHQGLS |
| | SPVTKSFNRGEC (SEQ ID NO: 146) |
| | VL: |
| | DIQLTQSPSSLSASVGDRVTITCSASQDISNYLNWYQQKPGKAPKVLIYFTSS |
| | LHSGVPSRFSGSGSGTDFTLTISSLQPEDFATYYCQQYSTVPWTFGQGTKVEI |
| | K (SEQ ID NO: 147) |
| | VL CDR1: SASQDISNYLN (SEQ ID NO: 128) |
| | VL CDR2: FTSSLHS (SEQ ID NO: 129) |
| | VL CDR3: QQYSTVPWT (SEQ ID NO: 130) |
| Ranibizumab | HC: |
| heavy | EVQLVESGGGLVQPGGSLRLSCAASGYDFTHYGMNWVRQAPGKGLEW |
| chain | VGWINTYTGEPTYAADFKRRFTFSLDTSKSTAYLQMNSLRAEDTAVYY |
| (VH in | CAKYPYYYGTSHWYFDVWGQGTLVTVSSASTKGPSVFPLAPSSKSTSGGT |
| Bold) | AALGCLVKDYFPEPVTVSWNSGALTSGVHTFPAVLQSSGLYSLSSVVTVPSS |
| | SLGTQTYICNVNHKPSNTKVDKKVEPKSCDKTHL (SEQ ID NO: 150) |
| | VH: |
| | EVQLVESGGGLVQPGGSLRLSCAASGYDFTHYGMNWVRQAPGKGLEWVG |
| | WINTYTGEPTYAADFKRRFTFSLDTSKSTAYLQMNSLRAEDTAVYYCAKYP |
| | YYYGTSHWYFDVWGQGTLVTV (SEQ ID NO: 142) |
| | VH CDR1: HYGMN (SEQ ID NO: 143) |
| | VH CDR2: WINTYTGEPTYAADFKR (SEQ ID NO: 144) |
| | VH CDR3: YPYYYGTSHWYFDV (SEQ ID NO: 125) |
| Ranibizumab | LC: |
| light | DIQLTQSPSSLSASVGDRVTITCSASQDISNYLNWYQQKPGKAPKVLIYF |
| chain | TSSLHSGVPSRFSGSGSGTDFTLTISSLQPEDFATYYCQQYSTVPWTFGQ |
| (VL in | GTKVEIKRTVAAPSVFIFPPSDEQLKSGTASVVCLLNNFYPREAKVQWKVD |
| Bold) | NALQSGNSQESVTEQDSKDSTYSLSSTLTLSKADYEKHKVYACEVTHQGLS |
| | SPVTKSFNRGEC (SEQ ID NO: 146) |
| | VL: |
| | DIQLTQSPSSLSASVGDRVTITCSASQDISNYLNWYQQKPGKAPKVLIYF |
| | TSSLHSGVPSRFSGSGSGTDFTLTISSLQPEDFATYYCQQYSTVPWTFGQ |
| | GTKVEIK (SEQ ID NO: 147) |
| | VL CDR1: SASQDISNYLN (SEQ ID NO: 128) |
| | VL CDR2: FTSSLHS (SEQ ID NO: 129) |
| | VL CDR3: QQYSTVPWT (SEQ ID NO: 130) |
| AI-081 | VH: |
| VH | EVQLVESGGGLVQPGGSLRLSCAASGYTFTNYGMNWVRQAPGKGLEWVG |
| | WINTYTGEPTYAADFKRRFTFSLDTSKSTAYLQMNSLRAEDTAVYYCAKYP |
| | HYYGSSHWYFDVWGQGTLVTVSS (SEQ ID NO: 122) |
| | VH CDR1: NYGMN (SEQ ID NO: 145) |
| | VH CDR2: WINTYTGEPTYAADFKR (SEQ ID NO: 144) |
| | VH CDR3: YPHYYGSSHWYFDV (SEQ ID NO: 125) |
| AI-081 | VL: |
| VL | DIQMTQSPSSLSASVGDRVTITCSASQDISNYLNWYQQKPGKAPKVLIYFTSS |
| | LHSGVPSRFSGSGSGTDFTLTISSLQPEDFATYYCQQYSTVPWTFGQGTKVEI |
| | K (SEQ ID NO: 127) |
| | VL CDR1: SASQDISNYLN (SEQ ID NO: 128) |
| | VL CDR2: FTSSLHS (SEQ ID NO: 129) |
| | VL CDR3: QQYSTVPWT (SEQ ID NO: 130) |
| HLX-04 | VH: |
| VH | DVQLVQSGVEVKNPGASVKVSCRASGYSFTNSGINWVKQAPGKGLKWMG |
| | WINTYTGEPTYADDFKGRFAFSLETSASSAYLQINNLKNEDTATYFCARFGD |
| | GYYWFFDVWGAGTTV (SEQ ID NO: 152) |
| | VH CDR1: GYSFTNSGIN (SEQ ID NO: 153) |
| | VH CDR2: INTYTGEPTYADDF (SEQ ID NO: 154) |
| | VH CDR3: FGDGYYWFFD (SEQ ID NO: 155) |
| HLX-04 | VL: |
| VL | DIVMTQSPDSLAVSLGERATINCKSSQSLLNSRTRKNFLAWYQQKPGQSPKL |
| | LIYWASTRESGVPDRFTGSGSGTDFTLTISSVQAEDLAVYYCKQSYNLYTFG |
| | GGTNLEIK (SEQ ID NO: 157) |
| | VL CDR1: KSSQSLLNSRTRKNFLA (SEQ ID NO: 158) |
| | VL CDR2: WASTRES (SEQ ID NO: 159) |
| | VL CDR3: KQSYNLYTFGG (SEQ ID NO: 160) |
| IBI305 | HC: |
| heavy | EVQLVESGGGLVQPGGSLRLSCAASGYTFTNYGMNWVRQAPGKGLEWVG |
| chain | WINTYTGEPTYAADFKRRFTFSLDTSKSTAYLQMNSLRAEDTAVYYCAKYP |
| | HYYGSSHWYFDVWGQGTLVTVSSASTKGPSVFPLAPSSKSTSGGTAALGCL |
| | VKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQVYT |
| | LPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK (SEQ ID |
| | NO: 121) |
| | VH: |
| | EVQLVESGGGLVQPGGSLRLSCAASGYTFTNYGMNWVRQAPGKGLEWVG |
| | WINTYTGEPTYAADFKRRFTFSLDTSKSTAYLQMNSLRAEDTAVYYCAKYP |
| | HYYGSSHWYFDVWGQGTLVTVSS (SEQ ID NO: 122) |
| | VH CDR1: NYGMN (SEQ ID NO: 145) |
| | VH CDR2: WINTYTGEPTYAADFKR (SEQ ID NO: 144) |
| | VH CDR3: YPHYYGSSHWYFDV (SEQ ID NO: 125) |
| IBI305 | LC: |
| light | DIQMTQSPSSLSASVGDRVTITCSASQDISNYLNWYQQKPGKAPKVLIYFTSS |
| chain | LHSGVPSRFSGSGSGTDFTLTISSLQPEDFATYYCQQYSTVPWTFGQGTKVEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC (SEQ ID NO: 126) |
| | VL: |
| | DIQMTQSPSSLSASVGDRVTITCSASQDISNYLNWYQQKPGKAPKVLIYFTSS |
| | LHSGVPSRFSGSGSGTDFTLTISSLQPEDFATYYCQQYSTVPWTFGQGTKVEI |
| | K (SEQ ID NO: 127) |
| | VL CDR1: SASQDISNYLN (SEQ ID NO: 128) |
| | VL CDR2: FTSSLHS (SEQ ID NO: 129) |
| | VL CDR3: QQYSTVPWT (SEQ ID NO: 130) |
| Bevacizumab | EVQLVESGGGLVQPGGSLRLSCAASGYTFTNYGMNWVRQAPGKGLEWVG |
| scFv | WINTYTGEPTYAADFKRRFTFSLDTSKSTAYLQMNSLRAEDTAVYYCAKYP |
| VH-VL | HYYGSSHWYFDVWGQGTLVTVSSGGGGSGGGGSGGGGSGGGGSDIQMTQ |
| | SPSSLSASVGDRVTITCSASQDISNYLNWYQQKPGKAPKVLIYFTSSLHSGVP |
| | SRFSGSGSGTDFTLTISSLQPEDFATYYCQQYSTVPWTFGQGTKVEIK (SEQ |
| | ID NO: 112) |
| Bevacizumab | DIQMTQSPSSLSASVGDRVTITCSASQDISNYLNWYQQKPGKAPKVLIYFTSS |
| scFv | LHSGVPSRFSGSGSGTDFTLTISSLQPEDFATYYCQQYSTVPWTFGQGTKVEI |
| VL-VH | KGGGGSGGGGSGGGGSGGGGSEVQLVESGGGLVQPGGSLRLSCAASGYTF |
| | TNYGMNWVRQAPGKGLEWVGWINTYTGEPTYAADFKRRFTFSLDTSKSTA |
| | YLQMNSLRAEDTAVYYCAKYPHYYGSSHWYFDVWGQGTLVTVSS (SEQ |
| | ID NO: 119) |
| Non- | EVQLVESGGGLVQPGGSLRLSCAASGYTFTNYGMNWVRQAPGKGLEW |
| PD1 | VGWINTYTGEPTYAADFKRRFTFSLDTSKSTAYLQMNSLRAEDTAVYY |
| targeted | CAKYPHYYGSSHWYFDVWGQGTLVTVSSASTKGPSVFPLAPSSKSTSGGT |
| ivonescimab | AALGCLVKDYFPEPVTVSWNSGALTSGVHTFPAVLQSSGLYSLSSVVTVPSS |
| HC | SLGTQTYICNVNHKPSNTKVDKKVEPKSCDKTHTCPPCPAPEAAGGPSVFLF |
| (VH in | PPKPKDTLMISRTPEVTCVVVDVSHEDPEVKFNWYVDGVEVHNAKTKPREE |
| bold) | QYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK |
| | (SEQ ID NO: 101) |
| Non- | DIQMTQSPSSLSASVGDRVTITCSASQDISNYLNWYQQKPGKAPKVLIYF |
| PD1 | TSSLHSGVPSRFSGSGSGTDFTLTISSLQPEDFATYYCQQYSTVPWTFGQ |
| targeted | GTKVEIKRTVAAPSVFIFPPSDEQLKSGTASVVCLLNNFYPREAKVQWKVD |
| ivonescimab | NALQSGNSQESVTEQDSKDSTYSLSSTLTLSKADYEKHKVYACEVTHQGLS |
| LC | SPVTKSFNRGEC (SEQ ID NO: 126) |
| (VL in | |
| bold) | |

TABLE 2B
Single Domain anti-VEGFA Antibodies

| | |
|---|---|
| Anti- | EVQLLVSGGGLVDPGGSLRLSCAASGFTFKAYPMMWVRQAPGKGLEWVSE |
| VEGF | ISPSGSYTYYADSVRGRFFISRDNSKNTLYLQMNSLRAEDTAVYYCAKDPRK |
| VHH 6 | LDYWGQGTLWTVSS (SEQ ID NO: 201) |
| | VH CDR1: AYPMM (SEQ ID NO: 202) |
| | VH CDR2: EISPSGSYTYYADSVRG (SEQ ID NO: 203) |
| | VH CDR3: DPRKLDY (SEQ ID NO: 204) |
| Anti- | EVQLLESGGGLVQPGGSLRLSCAASGFTFHLYDMMWVRQAPGKGLEWVSF |
| VEGF | IGGDGLNTYYADSVKGRFTISRDNSKNTLYLQMNSLRAEDTAVYYCAKAG |
| VHH | TQFDYWGQGTLVTVSS (SEQ ID NO: 205) |
| 190 | VH CDR1: LYDMM (SEQ ID NO: 206) |
| | VH CDR2: FIGGDGLNTYYADSVKG (SEQ ID NO: 207) |
| | VH CDR3: AGTQFDY (SEQ ID NO: 208) |
| Anti- | EVQLLESGGGLVQPGGSLRLSCAASGFTFQWYPMWWVRQAPGKGLEWVS |
| VEGF | LIEGQGDRTYYADSVKGRFTISRDNSKNTLYLQMNSLRAEDTAVYYCAKAG |
| VHH | DRTAGSRGNSFDYWGQGTLVTVSS (SEQ ID NO: 209) |
| 191 | VH CDR1: WYPMW (SEQ ID NO: 210) |
| | VH CDR2: LIEGQGDRTYYADSVKG (SEQ ID NO: 211) |
| | VH CDR3: AGDRTAGSRGNSFDY (SEQ ID NO: 212) |
| Anti- | EVQLLESGGGLVQPGGSLRLSCAASGFTFGAYPMMWVRQAPGKGLEWVSE |
| VEGF | ISPSGSYTYYADSVKGRFTISRDNSKNTLYLQMNSLRAEDTAVYYCAKDPR |
| VHH | KFDYWGQGTLVTVSS (SEQ ID NO: 213) |
| 192 | VH CDR1: AYPMM (SEQ ID NO: 202) |
| | VH CDR2: EISPSGSYTYYADSVKG (SEQ ID NO: 215) |
| | VH CDR3: DPRKFDY (SEQ ID NO: 216) |
| Anti- | DVQLVESGGGLVQPGGSLRLSCAASGRTFSSYSMGWFRQAPGKEREFVVAI |
| VEGF | SKGGYKYDAVSLEGRFTISRDNAKNTVYLQMNSLRPEDTAVYYCASSRAY |
| VHH 5 | GSSRLRLADTYEYWGQGTLVTVSS (SEQ ID NO: 217) |
| | VH CDR1: SYSMG (SEQ ID NO: 218) |
| | VH CDR2: AISKGGYKYDAVSLEG (SEQ ID NO: 219) |
| | VH CDR3: SRAYGSSRLRLADTYEY (SEQ ID NO: 220) |
| Anti- | DVQLVESGGGLVQPGGSLRLSCAASGRTFSSYSMGWFRQAPGKEREFVVAI |
| VEGF | SKGGYKYDAVSLEGRFTISRDNAKNTVYLQINSLRPEDTAVYYCASSRAYG |
| VHH | SSRLRLADTYEYWGQGTLVTVSS (SEQ ID NO: 221) |
| 175 | VH CDR1: SYSMG (SEQ ID NO: 218) |
| | VH CDR2: AISKGGYKYDAVSLEG (SEQ ID NO: 219) |
| | VH CDR3: SRAYGSSRLRLADTYEY (SEQ ID NO: 220) |
| Anti- | DIQMTQSPSSLSASVGDRVTITCRASQWIGPELSWYQQKPGKAPKLLIYHTSI |
| VEGF | LQSGVPSRFSGSGSGTDFTLTISSLQPEDFATYYCQQYMFQPRTFGQGTKVEI |
| sdAb | RR (SEQ ID NO: 222) |
| VL | VL CDR1: RASQWIGPELS (SEQ ID NO: 223) |
| | VL CDR2: HTSILQS (SEQ ID NO: 224) |
| | VL CDR3: QQYMFQPRT (SEQ ID NO: 225) |
| anti | EVQLVESGGGVVQPGGSLRLSCAASGRTFSSYSMGWFRQAPGKGLEFVVAI |
| VEGF | SKGGYKYDAVSLEGRFTISRDNSKNTVYLQMNSLRAEDTAVYYCASSRAY |
| VHH184 | GSSRLRLADTYEYWGQGTLVTVSS (SEQ ID NO: 280) |
| | VH CDR1: SYSMG (SEQ ID NO: 218) |
| | VH CDR2: AISKGGYKYDAVSLEG (SEQ ID NO: 219 |
| | VH CDR3: SRAYGSSRLRLADTYEY (SEQ ID NO: 220) |
| anti | EVQLVESGGGVVQPGGSLRLSCAASGRTFSSYSMGWFRQAPGKEREFVVAI |
| VEGF | SKGGYKYDAVSLEGRFTISRDNSKNTVYLQMNSLRAEDTAVYYCASSRAY |
| VHH185 | GSSRLRLADTYEYWGQGTLVTVSS (SEQ ID NO: 281) |
| | VH CDR1: SYSMG (SEQ ID NO: 218) |
| | VH CDR2: AISKGGYKYDAVSLEG (SEQ ID NO: 219) |
| | VH CDR3: SRAYGSSRLRLADTYEY (SEQ ID NO: 220) |
| anti | EVQLVESGGGLVQPGGSLRLSCAASGRTFSSYSMGWFRQAPGKEREFVVAI |
| VEGF | SKGGYKYDAVSVKGRFTISRDNSKNTVYLQMGSLRAEDMAVYYCASSRAY |
| VHH186 | GSSRLRLADTYEYWGQGTLVTVSS (SEQ ID NO: 282) |
| | VH CDR1: SYSMG (SEQ ID NO: 218) |
| | VH CDR2: AISKGGYKYDAVSVKG (SEQ ID NO: 279) |
| | VH CDR3: SRAYGSSRLRLADTYEY (SEQ ID NO: 220) |
| anti | EVQLVESGGGLVQPGGSLRLSCAASGRTFSSYSMGWFRQAPGKGLEFVVAI |
| VEGF | SKGGYKYDAVSVKGRFTISRDNSKNTVYLQMGSLRAEDMAVYYCASSRAY |
| VHH187 | GSSRLRLADTYEYWGQGTLVTVSS (SEQ ID NO: 283) |
| | VH CDR1: SYSMG (SEQ ID NO: 218) |
| | VH CDR2: AISKGGYKYDAVSVKG (SEQ ID NO: 279) |
| | VH CDR3: SRAYGSSRLRLADTYEY (SEQ ID NO: 220) |
| anti | EVQLVESGGGLVQPGGSLRLSCAASGRTFSSYSMGWFRQAPGKEREFVVAI |
| VEGF | SKGGYKYDAVSVKGRFTISRDNAKNTVYLQMNSLRAEDTAVYYCASSRAY |
| VHH188 | GSSRLRLADTYEYWGQGTLVTVSS (SEQ ID NO: 284) |
| | VH CDR1: SYSMG (SEQ ID NO: 218) |
| | VH CDR2: AISKGGYKYDAVSVKG (SEQ ID NO: 279) |
| | VH CDR3: SRAYGSSRLRLADTYEY (SEQ ID NO: 220) |
| anti | EVQLVESGGGLVQPGGSLRLSCAASGRTFSSYSMGWFRQAPGKEREFVVAI |
| VEGF | SKGGYKYDAVSVKGRFTISRDNAKNTVYLQMNSLRAEDTAVYYCASSRAY |
| VHH44 | GSSRLRLADTYEYWGQGTLVTVSS (SEQ ID NO: 284) |
| | VH CDR1: SYSMG (SEQ ID NO: 218) |
| | VH CDR2: AISKGGYKYDAVSVKG (SEQ ID NO: 279) |
| | VH CDR3: SRAYGSSRLRLADTYEY (SEQ ID NO: 220) |
| Anti | DVQLVESGGGLVQPGGSLRLSCAASGRTFSSYSMGWFRQAPGKEREFVVAI |
| VEGF | SKGGYKYDAVSLEGRFTISRDNAKNTVYLQINSLRPEDTAVYYCASSRAYG |
| VHH176 | SSRLRLADTYEYWGQGTLVTVSSPP (SEQ ID NO: 200) |
| | VH CDR1: SYSMG (SEQ ID NO: 218) |
| | VH CDR2: AISKGGYKYDAVSLEG (SEQ ID NO: 219) |
| | VH CDR3: SRAYGSSRLRLADTYEY (SEQ ID NO: 220) |
| Anti | EVQLVESGGGLVQPGGSLRLSCAASGRTFSSYSMGWFRQAPGKGLEFVVAI |
| VEGF | SKGGYKYDAVSVKGRFTISRDNAKNSVYLQMNSLRAEDTAVYYCASSRAY |
| VHH819 | GSSRLRLADTYEYWGQGTLVTVSS (SEQ ID NO: 214) |
| | VH CDR1: SYSMG (SEQ ID NO: 218) |
| | VH CDR2: AISKGGYKYDAVSVKG (SEQ ID NO: 279) |
| | VH CDR3: SRAYGSSRLRLADTYEY (SEQ ID NO: 220) |

In some embodiments, the anti-VEGFA binding domain is one provided in Table 2C, or a derivative thereof. In some embodiments, the anti-VEGFA binding domain is one which comprises the CDRs of a VHH provided in Table 2C. In some embodiments, the anti-VEGFA binding domain is one which comprises the CDRs of a VHH provided in Table 2C and comprises an amino acid sequence having at least 80%, 85%, 90%, 95%, 96%, 97%, 98%, 99%, or 100% identity to the corresponding full-length VHH sequence. In some embodiments, the anti-VEGFA binding domain is one of the VHHs provided in Table 2C. In some embodiments, the C-terminal “PP” of the VHH in Table 2 C can be omitted.

TABLE 2C
Exemplary anti-VEGFA VHH binding domains

| | | Full | | | | | | |
|---|---|---|---|---|---|---|---|---|
| Anti- | | Sequence | | CDR1 | | CDR2 | | CDR3 |
| VEGF | | SEQ | | SEQ | | SEQ | | SEQ |
| VHH | | ID | | ID | | ID | | ID |
| No. | Full Sequence | NO | CDR1 | NO | CDR2 | NO | CDR3 | NO |
| VHH127 | EVQLVESGGGLV | 300 | GISFS | 301 | IGLS | 302 | ARGH | 303 |
| | QPGGSLRLSCAAS | | HYG | | GGST | | DFWS | |
| | GISFSHYGMAWY | | | | | | GLTP | |
| | RQAPGKGRELVA | | | | | | DY | |
| | GIGLSGGSTSYAD | | | | | | | |
| | SVKGRFTISRDNA | | | | | | | |
| | KNTLYLQMNSLR | | | | | | | |
| | PEDTAVYYCARG | | | | | | | |
| | HDFWSGLTPDYW | | | | | | | |
| | GQGTLVTVSSPP | | | | | | | |
| VHH128 | EVQLVESGGGLV | 304 | GFTV | 305 | ISPSG | 306 | AKTG | 307 |
| | QPGGSLRLSCAAS | | SSGG | | VTT | | GSSG | |
| | GFTVSSGGMGWF | | | | | | SWFP | |
| | RQAPGKGRELVA | | | | | | DDAF | |
| | AISPSGVTTYYPD | | | | | | DV | |
| | SVEGRFTISRDNA | | | | | | | |
| | KRMVYLQMNSL | | | | | | | |
| | RAEDTAVYYCAK | | | | | | | |
| | TGGSSGSWFPDD | | | | | | | |
| | AFDVWGQGTMV | | | | | | | |
| | TVSSPP | | | | | | | |
| VHH129 | EVQLVESGGGLV | 308 | GFPS | 309 | MSGT | 310 | ANR | 311 |
| | QPGGSLRLSCAAS | | SSHG | | GATT | | WWY | |
| | GFPSSSHGMAWY | | | | | | CSGG | |
| | RQAPGKGRELVA | | | | | | SCYD | |
| | GMSGTGATTSYA | | | | | | VDY | |
| | DSVKGRFTISRDN | | | | | | | |
| | AKNTLYLQMNSL | | | | | | | |
| | RPEDTAVYYCAN | | | | | | | |
| | RWWYCSGGSCY | | | | | | | |
| | DVDYWGQGTLV | | | | | | | |
| | TVSSPP | | | | | | | |
| VHH130 | EVQLVESGGGLV | 312 | GFTF | 313 | ISAT | 314 | ATVF | 315 |
| | QPGGSLRLSCAAS | | GVY | | GGST | | CSGG | |
| | GFTFGVYDMGW | | D | | | | SCYP | |
| | LRQAPGKEREFV | | | | | | FGY | |
| | AAISATGGSTYYA | | | | | | | |
| | DSVKGRFTISRDN | | | | | | | |
| | SKNTLYLQMNSL | | | | | | | |
| | RPEDTAVYYCAT | | | | | | | |
| | VFCSGGSCYPFGY | | | | | | | |
| | WGQGTLVTVSSP | | | | | | | |
| | P | | | | | | | |
| VHH131 | DVQLVESGGGLV | 316 | GLTF | 317 | ITGS | 318 | ARDR | 319 |
| | QPGGSLRLSCAAS | | STSE | | GAFT | | CSSS | |
| | GLTFSTSEMGWF | | | | | | NCVD | |
| | RQAPGKEREFVA | | | | | | DFDI | |
| | GITGSGAFTYYVD | | | | | | | |
| | SLKGRFTISRDNS | | | | | | | |
| | KNTVYLQMNSLR | | | | | | | |
| | PEDTAVYYCARD | | | | | | | |
| | RCSSSNCVDDFDI | | | | | | | |
| | WGPGTMVTVSSP | | | | | | | |
| | P | | | | | | | |
| VHH132 | EVQLVESGGGLV | 320 | GFTG | 321 | ISGN | 322 | ARGL | 323 |
| | QPGGSLRLSCAAS | | SSTY | | ADYT | | YSYD | |
| | GFTGSSTYMAWY | | | | | | SSPF | |
| | RQAPGKGRELVA | | | | | | DY | |
| | GISGNADYTSYA | | | | | | | |
| | DSVKGRFTISRDN | | | | | | | |
| | AKNTLYLQMNSL | | | | | | | |
| | RPEDTAVYYCAR | | | | | | | |
| | GLYSYDSSPFDY | | | | | | | |
| | WGQGTLVTVSSP | | | | | | | |
| | P | | | | | | | |
| VHH133 | EVQLVESGGGLV | 324 | GFTV | 325 | ISGT | 326 | ARGV | 327 |
| | QPGGSLRLSCAAS | | SHYG | | GAYT | | LGPY | |
| | GFTVSHYGMAW | | | | | | GDYP | |
| | YRQAPGKGRELV | | | | | | YWY | |
| | AGISGTGAYTSYA | | | | | | FDL | |
| | DSVKGRFTISKDN | | | | | | | |
| | AKNTLYLQMNSL | | | | | | | |
| | RPEDTAVYYCAR | | | | | | | |
| | GVLGPYGDYPYW | | | | | | | |
| | YFDLWGRGTLVT | | | | | | | |
| | VSSPP | | | | | | | |
| VHH134 | EVQLVESGGGLV | 328 | GFSV | 329 | ISSG | 330 | ARS | 331 |
| | QPGGSLRLSCAAS | | SGHG | | GTVT | | WYC | |
| | GFSVSGHGMAW | | | | | | SGGS | |
| | YRQAPGKGRELV | | | | | | CYPD | |
| | AGISSGGTVTSYA | | | | | | Y | |
| | DSVKGRFTISRDN | | | | | | | |
| | AKNTLYLQMNSL | | | | | | | |
| | RPEDTAVYYCAR | | | | | | | |
| | SWYCSGGSCYPD | | | | | | | |
| | YWGQGTLVTVSS | | | | | | | |
| | PP | | | | | | | |
| VHH135 | DVQLVESGGGLV | 332 | GFSF | 333 | VTGS | 334 | AREA | 335 |
| | QPGGSLRLSCAAS | | TKYG | | GDAT | | VAGP | |
| | GFSFTKYGMGWF | | | | | | YYFD | |
| | RQAPGKEREFVA | | | | | | Y | |
| | GVTGSGDATYYV | | | | | | | |
| | DSLKGRFTISRDN | | | | | | | |
| | SKNTVYLQMNSL | | | | | | | |
| | RPEDTAVYYCAR | | | | | | | |
| | EAVAGPYYFDYW | | | | | | | |
| | GQGTLVTVSSPP | | | | | | | |
| VHH136 | EVQLVESGGGLV | 336 | GFDF | 337 | ISTSG | 338 | AREA | 339 |
| | QPGGSLRLSCAAS | | STYP | | GVT | | RGNY | |
| | GFDFSTYPMGWL | | | | | | AHFD | |
| | RQAPGKEREFVA | | | | | | Y | |
| | AISTSGGVTYYAD | | | | | | | |
| | SVKGRFTISRDNS | | | | | | | |
| | KNTLYLQMNSLR | | | | | | | |
| | PEDTAVYYCARE | | | | | | | |
| | ARGNYAHFDYW | | | | | | | |
| | GQGTLVTVSSPP | | | | | | | |
| VHH137 | EVQLVESGGGLV | 340 | GFGF | 341 | ISAG | 342 | AKG | 343 |
| | QPGGSLRLSCAAS | | SGSA | | STTT | | VDY | |
| | GFGFSGSAMGWL | | | | | | YGPL | |
| | RQAPGKEREFVA | | | | | | PFDY | |
| | AISAGSTTTYYAD | | | | | | | |
| | SVKGRFTISRDNS | | | | | | | |
| | KNTLYLQMNSLR | | | | | | | |
| | PEDTAVYYCAKG | | | | | | | |
| | VDYYGPLPFDYW | | | | | | | |
| | GQGTLVTVSSPP | | | | | | | |
| VHH138 | EVQLVESGGGLV | 344 | GFSF | 345 | ISSTG | 346 | ARIY | 347 |
| | QPGGSLRLSCAAS | | GSYG | | GVT | | GPW | |
| | GFSFGSYGMGWL | | | | | | YFDL | |
| | RQAPGKEREFVA | | | | | | | |
| | AISSTGGVTYYAD | | | | | | | |
| | SVKGRFTISRDNS | | | | | | | |
| | KNTLYLQMNSLR | | | | | | | |
| | PEDTAVYYCARI | | | | | | | |
| | YGPWYFDLWGR | | | | | | | |
| | GTLVTVSSPP | | | | | | | |
| VHH139 | DVQLVESGGGLV | 348 | GFSF | 349 | ITGT | 350 | ARVG | 351 |
| | QPGGSLRLSCAAS | | GDHE | | GEYT | | YSSS | |
| | GFSFGDHEMGWF | | | | | | SYFD | |
| | RQAPGKEREFVA | | | | | | Y | |
| | GITGTGEYTYYV | | | | | | | |
| | DSLKGRFTISRDN | | | | | | | |
| | SKNTVYLQMNSL | | | | | | | |
| | RPEDTAVYYCAR | | | | | | | |
| | VGYSSSSYFDYW | | | | | | | |
| | GQGTLVTVSSPP | | | | | | | |
| VHH140 | DVQLVESGGGLV | 352 | GFTF | 353 | IGGS | 354 | ARAA | 355 |
| | QPGGSLRLSCAAS | | SDAE | | VSST | | VAGP | |
| | GFTFSDAEMGWF | | | | | | YYFD | |
| | RQAPGKEREFVA | | | | | | Y | |
| | GIGGSVSSTYYVD | | | | | | | |
| | SLKGRFTISRDNS | | | | | | | |
| | KNTVYLQMNSLR | | | | | | | |
| | PEDTAVYYCARA | | | | | | | |
| | AVAGPYYFDYW | | | | | | | |
| | GQGTLVTVSSPP | | | | | | | |
| VHH141 | EVQLVESGGGLV | 356 | GIAF | 357 | IIGSG | 358 | TTAA | 359 |
| | QPGGSLRLSCAAS | | SNHA | | AGT | | ATND | |
| | GIAFSNHAMGWF | | | | | | Y | |
| | RQAPGKGRELVA | | | | | | | |
| | AIIGSGAGTYYPD | | | | | | | |
| | SVEGRFTISRDNA | | | | | | | |
| | KRMVYLQMNSL | | | | | | | |
| | RAEDTAVYYCTT | | | | | | | |
| | AAATNDYWGQG | | | | | | | |
| | TLVTVSSPP | | | | | | | |
| VHH142 | EVQLVESGGGLV | 360 | GFGF | 361 | ISTSG | 362 | ASLI | 363 |
| | QPGGSLRLSCAAS | | STYD | | TWT | | YGD | |
| | GFGFSTYDMAWY | | | | | | YEAG | |
| | RQAPGKGRELVA | | | | | | LDY | |
| | GISTSGTWTSYAD | | | | | | | |
| | SVKGRFTISRDNA | | | | | | | |
| | KNTLYLQMNSLR | | | | | | | |
| | PEDTAVYYCASLI | | | | | | | |
| | YGDYEAGLDYW | | | | | | | |
| | GQGTLVTVSSPP | | | | | | | |
| VHH143 | DVQLVESGGGLV | 364 | GFSF | 365 | IGSG | 366 | ARVA | 367 |
| | QPGGSLRLSCAAS | | GDY | | GST | | VSGT | |
| | GFSFGDYDMGWF | | D | | | | TRDY | |
| | RQAPGKEREFVA | | | | | | FDY | |
| | GIGSGGSTYYVDS | | | | | | | |
| | LKGRFTISRDNSK | | | | | | | |
| | NTVYLQMNSLRP | | | | | | | |
| | EDTAVYYCARVA | | | | | | | |
| | VSGTTRDYFDYW | | | | | | | |
| | GQGSLVTVSSPP | | | | | | | |
| VHH144 | DVQLVESGGGLV | 368 | GFVF | 369 | ITGST | 370 | ARVG | 371 |
| | QPGGSLRLSCAAS | | SDYD | | GGT | | AGGP | |
| | GFVFSDYDMGWF | | | | | | EWFD | |
| | RQAPGKEREFVA | | | | | | Y | |
| | GITGSTGGTYYV | | | | | | | |
| | DSLKGRFTISRDN | | | | | | | |
| | SKNTVYLQMNSL | | | | | | | |
| | RPEDTAVYYCAR | | | | | | | |
| | VGAGGPEWFDY | | | | | | | |
| | WGQGTLVTVSSP | | | | | | | |
| | P | | | | | | | |
| VHH145 | DVQLVESGGGLV | 372 | GFDF | 373 | VSGG | 374 | AREC | 375 |
| | QPGGSLRLSCAAS | | SNHA | | AGGT | | SGGS | |
| | GFDFSNHAMGWF | | | | | | CSNY | |
| | RQAPGKEREFVA | | | | | | FDY | |
| | GVSGGAGGTYYV | | | | | | | |
| | DSLKGRFTISRDN | | | | | | | |
| | SKNTVYLQMNSL | | | | | | | |
| | RPEDTAVYYCAR | | | | | | | |
| | ECSGGSCSNYFD | | | | | | | |
| | YWGQGTLVTVSS | | | | | | | |
| | PP | | | | | | | |
| VHH146 | EVQLVESGGGLV | 376 | GFTS | 377 | ISGP | 378 | ARFR | 379 |
| | QPGGSLRLSCAAS | | SDHY | | GVST | | PVGA | |
| | GFTSSDHYMAWY | | | | | | IPFDL | |
| | RQAPGKGRELVA | | | | | | | |
| | GISGPGVSTSYAD | | | | | | | |
| | SVKGRFTISRDNA | | | | | | | |
| | KNTLYLQMNSLR | | | | | | | |
| | PEDTAVYYCARF | | | | | | | |
| | RPVGAIPFDLWG | | | | | | | |
| | RGTLVTVSSPP | | | | | | | |
| VHH147 | EVQLVESGGGLV | 380 | GFNF | 381 | MSGS | 382 | ARGI | 383 |
| | QPGGSLRLSCAAS | | QDFA | | GSVT | | AAA | |
| | GFNFQDFAMGWF | | | | | | GPTV | |
| | RQAPGKGRELVA | | | | | | DEN | |
| | AMSGSGSVTYYP | | | | | | WFDP | |
| | DSVEGRFTISRDN | | | | | | | |
| | AKRMVYLQMNS | | | | | | | |
| | LRAEDTAVYYCA | | | | | | | |
| | RGIAAAGPTVDE | | | | | | | |
| | NWFDPWGQGTL | | | | | | | |
| | VTVSSPP | | | | | | | |
| VHH148 | EVQLVESGGGLV | 384 | GFSF | 385 | ISVS | 386 | ARET | 387 |
| | QPGGSLRLSCAAS | | KTYD | | GGW | | AAGE | |
| | GFSFKTYDMGWF | | | | T | | IDY | |
| | RQAPGKGRELVA | | | | | | | |
| | AISVSGGWTYYP | | | | | | | |
| | DSVEGRFTISRDN | | | | | | | |
| | AKRMVYLQMNS | | | | | | | |
| | LRAEDTAVYYCA | | | | | | | |
| | RETAAGEIDYWG | | | | | | | |
| | QGTLVTVSSPP | | | | | | | |
| VHH149 | EVQLVESGGGLV | 388 | GFSF | 389 | ISSA | 390 | ARDR | 391 |
| | QPGGSLRLSCAAS | | NPYS | | GGHT | | NLYS | |
| | GFSFNPYSMGWL | | | | | | SSLD | |
| | RQAPGKEREFVA | | | | | | AFDI | |
| | AISSAGGHTYYA | | | | | | | |
| | DSVKGRFTISRDN | | | | | | | |
| | SKNTLYLQMNSL | | | | | | | |
| | RPEDTAVYYCAR | | | | | | | |
| | DRNLYSSSLDAFD | | | | | | | |
| | IWGQGTMVTVSS | | | | | | | |
| | PP | | | | | | | |
| VHH150 | EVQLVESGGGLV | 392 | GFTF | 393 | ISGP | 394 | ARDE | 395 |
| | QPGGSLRLSCAAS | | ATHS | | GDFT | | EFDY | |
| | GFTFATHSMGWF | | | | | | ELDY | |
| | RQAPGKGRELVA | | | | | | | |
| | AISGPGDFTYYPD | | | | | | | |
| | SVEGRFTISRDNA | | | | | | | |
| | KRMVYLQMNSL | | | | | | | |
| | RAEDTAVYYCAR | | | | | | | |
| | DEEFDYELDYWG | | | | | | | |
| | QGTLVTVSSPP | | | | | | | |
| VHH151 | EVQLVESGGGLV | 396 | GFTL | 397 | IGGS | 398 | ARDI | 399 |
| | QPGGSLRLSCAAS | | PTYG | | GDW | | GSYA | |
| | GFTLPTYGMGWL | | | | T | | Y | |
| | RQAPGKEREFVA | | | | | | | |
| | AIGGSGDWTYYA | | | | | | | |
| | DSVKGRFTISRDN | | | | | | | |
| | SKNTLYLQMNSL | | | | | | | |
| | RPEDTAVYYCAR | | | | | | | |
| | DIGSYAYWGQGI | | | | | | | |
| | RVTVSSPP | | | | | | | |
| VHH152 | EVQLVESGGGLV | 400 | GFAF | 401 | ITGS | 402 | ARDE | 403 |
| | QPGGSLRLSCAAS | | STSA | | GGM | | EFTS | |
| | GFAFSTSAMGWF | | | | T | | SFDY | |
| | RQAPGKGRELVA | | | | | | | |
| | AITGSGGMTYYP | | | | | | | |
| | DSVEGRFTISRDN | | | | | | | |
| | AKRMVYLQMNS | | | | | | | |
| | LRAEDTAVYYCA | | | | | | | |
| | RDEEFTSSFDYW | | | | | | | |
| | GQGTLVTVSSPP | | | | | | | |
| VHH153 | EVQLVESGGGLV | 404 | GFSL | 405 | IGAS | 406 | ARH | 407 |
| | QPGGSLRLSCAAS | | DEYG | | GHST | | WLG | |
| | GFSLDEYGMAW | | | | | | WSSG | |
| | YRQAPGKGRELV | | | | | | YPDY | |
| | AGIGASGHSTSYA | | | | | | | |
| | DSVKGRFTISRDN | | | | | | | |
| | AKNTLYLQMNSL | | | | | | | |
| | RPEDTAVYYCAR | | | | | | | |
| | HWLGWSSGYPD | | | | | | | |
| | YWGQGTLVTVSS | | | | | | | |
| | PP | | | | | | | |
| VHH154 | EVQLVESGGGLV | 408 | GFIFS | 409 | ISESG | 410 | ARFP | 411 |
| | QPGGSLRLSCAAS | | GAD | | DWT | | DAAP | |
| | GFIFSGADMGWF | | | | | | GV | |
| | RQAPGKGRELVA | | | | | | | |
| | AISESGDWTYYP | | | | | | | |
| | DSVEGRFTISRDN | | | | | | | |
| | AKRMVYLQMNS | | | | | | | |
| | LRAEDTAVYYCA | | | | | | | |
| | RFPDAAPGVWGK | | | | | | | |
| | GTTVTVSSPP | | | | | | | |
| VHH155 | EVQLVESGGGLV | 412 | GFTF | 413 | ISGG | 414 | AILE | 415 |
| | QPGGSLRLSCAAS | | SNVD | | HGDT | | TGDY | |
| | GFTFSNVDMGWL | | | | | | | |
| | RQAPGKEREFVA | | | | | | | |
| | AISGGHGDTYYA | | | | | | | |
| | DSVKGRFTISRDN | | | | | | | |
| | SKNTLYLQMNSL | | | | | | | |
| | RPEDTAVYYCAIL | | | | | | | |
| | ETGDYWGQGTLV | | | | | | | |
| | TVSSPP | | | | | | | |
| VHH156 | DVQLVESGGGLV | 416 | GFLF | 417 | VSHS | 418 | ARTA | 419 |
| | QPGGSLRLSCAAS | | GDY | | GSST | | VAGP | |
| | GFLFGDYDMGWF | | D | | | | AYFD | |
| | RQAPGKEREFVA | | | | | | Y | |
| | GVSHSGSSTYYV | | | | | | | |
| | DSLKGRFTISRDN | | | | | | | |
| | SKNTVYLQMNSL | | | | | | | |
| | RPEDTAVYYCAR | | | | | | | |
| | TAVAGPAYFDYW | | | | | | | |
| | GQGTLVTVSSPP | | | | | | | |
| VHH157 | DVQLVESGGGLV | 420 | GFNF | 421 | ISGS | 422 | ARVG | 423 |
| | QPGGSLRLSCAAS | | EEYG | | GSGT | | VTTV | |
| | GFNFEEYGMGWF | | | | | | TYFD | |
| | RQAPGKEREFVA | | | | | | Y | |
| | GISGSGSGTYYVD | | | | | | | |
| | SLKGRFTISRDNS | | | | | | | |
| | KNTVYLQMNSLR | | | | | | | |
| | PEDTAVYYCARV | | | | | | | |
| | GVTTVTYFDYWG | | | | | | | |
| | QGTLVTVSSPP | | | | | | | |
| VHH158 | EVQLVESGGGLV | 424 | GFTV | 425 | ITGST | 426 | ARDT | 427 |
| | QPGGSLRLSCAAS | | SNHF | | GST | | FSSG | |
| | GFTVSNHFMGWL | | | | | | YPPD | |
| | RQAPGKEREFVA | | | | | | Y | |
| | AITGSTGSTYYAD | | | | | | | |
| | SVKGRFTISRDNS | | | | | | | |
| | KNTLYLQMNSLR | | | | | | | |
| | PEDTAVYYCARD | | | | | | | |
| | TFSSGYPPDYWG | | | | | | | |
| | QGTLVTVSSPP | | | | | | | |
| VHH159 | DVQLVESGGGLV | 428 | GFKF | 429 | ISVY | 430 | ARTA | 431 |
| | QPGGSLRLSCAAS | | STYD | | AGST | | VAGP | |
| | GFKFSTYDMGWF | | | | | | GYFD | |
| | RQAPGKEREFVA | | | | | | Y | |
| | GISVYAGSTYYV | | | | | | | |
| | DSLKGRFTISRDN | | | | | | | |
| | SKNTVYLQMNSL | | | | | | | |
| | RPEDTAVYYCAR | | | | | | | |
| | TAVAGPGYFDYW | | | | | | | |
| | GQGTLVTVSSPP | | | | | | | |
| VHH160 | DVQLVESGGGLV | 432 | GFPS | 433 | VSGS | 434 | AREP | 435 |
| | QPGGSLRLSCAAS | | SSYS | | GTST | | LSGS | |
| | GFPSSSYSMGWF | | | | | | TPDY | |
| | RQAPGKEREFVA | | | | | | FDY | |
| | GVSGSGTSTYYV | | | | | | | |
| | DSLKGRFTISRDN | | | | | | | |
| | SKNTVYLQMNSL | | | | | | | |
| | RPEDTAVYYCAR | | | | | | | |
| | EPLSGSTPDYFDY | | | | | | | |
| | WGQGTLVTVSSP | | | | | | | |
| | P | | | | | | | |
| VHH161 | EVQLVESGGGLV | 436 | GFSF | 437 | ISTSG | 438 | ARDT | 439 |
| | QPGGSLRLSCAAS | | HDH | | GFT | | LEPQ | |
| | GFSFHDHAMGWF | | A | | | | AFDI | |
| | RQAPGKGRELVA | | | | | | | |
| | AISTSGGFTYYPD | | | | | | | |
| | SVEGRFTISRDNA | | | | | | | |
| | KRMVYLQMNSL | | | | | | | |
| | RAEDTAVYYCAR | | | | | | | |
| | DTLEPQAFDIWG | | | | | | | |
| | QGTMVTVSSPP | | | | | | | |
| VHH162 | DVQLVESGGGLV | 440 | GFAV | 44 | IIGTG | 442 | ARVA | 443 |
| | QPGGSLRLSCAAS | | SSNE | | GST | | AAGT | |
| | GFAVSSNEMGWF | | | | | | GGD | |
| | RQAPGKEREFVA | | | | | | YFDY | |
| | GIIGTGGSTYYVD | | | | | | | |
| | SLKGRFTISRDNS | | | | | | | |
| | KNTVYLQMNSLR | | | | | | | |
| | PEDTAVYYCARV | | | | | | | |
| | AAAGTGGDYFDY | | | | | | | |
| | WGQGTLVTVSSP | | | | | | | |
| | P | | | | | | | |
| VHH163 | EVQLVESGGGLV | 444 | GLPF | 445 | IGGS | 398 | TAIT | 447 |
| | QPGGSLRLSCAAS | | SSYS | | GDW | | TTMD | |
| | GLPFSSYSMGWL | | | | T | | V | |
| | RQAPGKEREFVA | | | | | | | |
| | AIGGSGDWTYYA | | | | | | | |
| | DSVKGRFTISRDN | | | | | | | |
| | SKNTLYLQMNSL | | | | | | | |
| | RPEDTAVYYCTAI | | | | | | | |
| | TTTMDVWGQGT | | | | | | | |
| | TVTVSSPP | | | | | | | |
| VHH164 | DVQLVESGGGLV | 448 | GFDF | 449 | ITGST | 426 | ARVP | 451 |
| | QPGGSLRLSCAAS | | SSHE | | GST | | SAAG | |
| | GFDFSSHEMGWF | | | | | | RNYF | |
| | RQAPGKEREFVA | | | | | | DY | |
| | GITGSTGSTYYVD | | | | | | | |
| | SLKGRFTISRDNS | | | | | | | |
| | KNTVYLQMNSLR | | | | | | | |
| | PEDTAVYYCARV | | | | | | | |
| | PSAAGRNYFDYW | | | | | | | |
| | GQGTLVTVSSPP | | | | | | | |
| VHH165 | DVQLVESGGGLV | 452 | GFTL | 453 | ISSDE | 454 | ARVS | 455 |
| | QPGGSLRLSCAAS | | SSAG | | GGT | | IAGS | |
| | GFTLSSAGMGWF | | | | | | SYFD | |
| | RQAPGKEREFVA | | | | | | Y | |
| | GISSDEGGTYYVD | | | | | | | |
| | SLKGRFTISRDNS | | | | | | | |
| | KNTVYLQMNSLR | | | | | | | |
| | PEDTAVYYCARV | | | | | | | |
| | SIAGSSYFDYWG | | | | | | | |
| | QGTLVTVSSPP | | | | | | | |
| VHH166 | DVQLVESGGGLV | 456 | GFTL | 457 | ISGIG | 458 | ARVA | 459 |
| | QPGGSLRLSCAAS | | TDYE | | GTT | | ATGA | |
| | GFTLTDYEMGWF | | | | | | EYFQ | |
| | RQAPGKEREFVA | | | | | | H | |
| | GISGIGGTTYYVD | | | | | | | |
| | SLKGRFTISRDNS | | | | | | | |
| | KNTVYLQMNSLR | | | | | | | |
| | PEDTAVYYCARV | | | | | | | |
| | AATGAEYFQHW | | | | | | | |
| | GQGTLVTVSSPP | | | | | | | |
| VHH167 | DVQLVESGGGLV | 460 | GFSF | 461 | VSPS | 462 | ARVG | 463 |
| | QPGGSLRLSCAAS | | SSYD | | GGDT | | LVGA | |
| | GFSFSSYDMGWF | | | | | | TYFD | |
| | RQAPGKEREFVA | | | | | | Y | |
| | GVSPSGGDTYYV | | | | | | | |
| | DSLKGRFTISRDN | | | | | | | |
| | SKNTVYLQMNSL | | | | | | | |
| | RPEDTAVYYCAR | | | | | | | |
| | VGLVGATYFDY | | | | | | | |
| | WGQGTLVTVSSP | | | | | | | |
| | P | | | | | | | |
| VHH168 | DVQLVESGGGLV | 464 | GFSF | 465 | IDAS | 466 | AREA | 467 |
| | QPGGSLRLSCAAS | | STDA | | GGST | | AVA | |
| | GFSFSTDAMGWF | | | | | | GPYF | |
| | RQAPGKEREFVA | | | | | | FDY | |
| | GIDASGGSTYYV | | | | | | | |
| | DSLKGRFTISRDN | | | | | | | |
| | SKNTVYLQMNSL | | | | | | | |
| | RPEDTAVYYCAR | | | | | | | |
| | EAAVAGPYFFDY | | | | | | | |
| | WGQGALVTVSSP | | | | | | | |
| | P | | | | | | | |
| VHH169 | DVQLVESGGGLV | 468 | GFTL | 469 | ISGST | 470 | ARVG | 471 |
| | QPGGSLRLSCAAS | | TDYD | | PST | | KTGA | |
| | GFTLTDYDMGWF | | | | | | TYFD | |
| | RQAPGKEREFVA | | | | | | Y | |
| | GISGSTPSTYYVD | | | | | | | |
| | SLKGRFTISRDNS | | | | | | | |
| | KNTVYLQMNSLR | | | | | | | |
| | PEDTAVYYCARV | | | | | | | |
| | GKTGATYFDYW | | | | | | | |
| | GQGTLVTVSSPP | | | | | | | |
| VHH170 | EVQLVESGGGLV | 472 | GFTS | 473 | ISAG | 474 | ANH | 475 |
| | QPGGSLRLSCAAS | | TNYV | | GTDT | | QA | |
| | GFTSTNYVMGWF | | | | | | | |
| | RQAPGKGRELVA | | | | | | | |
| | AISAGGTDTYYPD | | | | | | | |
| | SVEGRFTISRDNA | | | | | | | |
| | KRMVYLQMNSL | | | | | | | |
| | RAEDTAVYYCAN | | | | | | | |
| | HQAVGQGTLVTV | | | | | | | |
| | SSPP | | | | | | | |
| VHH171 | EVQLVESGGGLV | 476 | GFDF | 477 | VSST | 478 | ARAD | 479 |
| | QPGGSLRLSCAAS | | SNAD | | GGTT | | VVTS | |
| | GFDFSNADMGWL | | | | | | DY | |
| | RQAPGKEREFVA | | | | | | | |
| | AVSSTGGTTYYA | | | | | | | |
| | DSVKGRFTISRDN | | | | | | | |
| | SKNTLYLQMNSL | | | | | | | |
| | RPEDTAVYYCAR | | | | | | | |
| | ADVVTSDYWGQ | | | | | | | |
| | GTLVTVSSPP | | | | | | | |
| VHH172 | DVQLVESGGGLV | 480 | GFTF | 481 | INPE | 482 | ARVG | 483 |
| | QPGGSLRLSCAAS | | TAYD | | GSET | | YSSS | |
| | GFTFTAYDMGWF | | | | | | SFFD | |
| | RQAPGKEREFVA | | | | | | Y | |
| | GINPEGSETYYVD | | | | | | | |
| | SLKGRFTISRDNS | | | | | | | |
| | KNTVYLQMNSLR | | | | | | | |
| | PEDTAVYYCARV | | | | | | | |
| | GYSSSSFFDYWG | | | | | | | |
| | QGTLVTVSSPP | | | | | | | |
| VHH173 | DVQLVESGGGLV | 484 | GFSF | 485 | ISGS | 486 | ARVA | 487 |
| | QPGGSLRLSCAAS | | TTSG | | GSST | | QAG | |
| | GFSFTTSGMGWF | | | | | | GEYF | |
| | RQAPGKEREFVA | | | | | | DY | |
| | GISGSGSSTYYVD | | | | | | | |
| | SLKGRFTISRDNS | | | | | | | |
| | KNTVYLQMNSLR | | | | | | | |
| | PEDTAVYYCARV | | | | | | | |
| | AQAGGEYFDYW | | | | | | | |
| | GQGTLVTVSSPP | | | | | | | |
| VHH174 | DVQLVESGGGLV | 488 | GFPF | 489 | LGGS | 490 | ARVG | 491 |
| | QPGGSLRLSCAAS | | SKFD | | GGST | | SVGA | |
| | GFPFSKFDMGWF | | | | | | TFFD | |
| | RQAPGKEREFVA | | | | | | Y | |
| | GLGGSGGSTYYV | | | | | | | |
| | DSLKGRFTISRDN | | | | | | | |
| | SKNTVYLQMNSL | | | | | | | |
| | RPEDTAVYYCAR | | | | | | | |
| | VGSVGATFFDYW | | | | | | | |
| | GQGTLVTVSSPP | | | | | | | |
| VHH190 | EVQLLESGGGLV | | | | | | | |
| | QPGGSLRLSCAAS | | | | | | | |
| | GFTFHLYDMMW | | | | | | | |
| | VRQAPGKGLEWV | | | | | | | |
| | SFIGGDGLNTYYA | | | | | | | |
| | DSVKGRFTISRDN | | | | | | | |
| | SKNTLYLQMNSL | | | | | | | |
| | RAEDTAVYYCAK | | | | | | | |
| | AGTQFDYWGQG | | | | | | | |
| | TLVTVSS | | | | | | | |
| VHH191 | | | | | | | | |
| VHH192 | | | | | | | | |

In some embodiments, the anti-VEGFA VHH of the immunocytokine composition comprises one or more modifications which imparts the VHH with reduced immunogenicity or reduced binding of pre-existing antibodies to the VHH. Examples of such modifications are described in, for example, U.S. Patent Publication Nos. US20180009888A9 (e.g., extension peptides of 1 to 5 extending beyond the C-terminal “SS” of the VHH, such as those consisting of the amino acids Ala and Gly), US20160207981A1 (e.g., substitutions of the C-terminal “SS” of the VHH, such as with an amino acid or peptide of a sequence E, SE, EG, SEG, EP, EPG, DP, DPG, K, SK, KP, KPG, RP, or RPG and/or substitutions of Leu 11 of the VHH, such as an L11K, L11R, L11D, or L11E substitution), US20140161796A1 (e.g., deletions of certain sequences from the VHH), US20170121399A1 (e.g., substitutions of Leu 11 of the VHH (e.g., L11K or L11V) and/or Leu 89 (e.g., L89T)), and Lin et al., “A structure-based engineering approach to abrogate pre-existing antibody binding to biotherapeutics,” PLoS ONE 16(7): e0254944. doi.org/10.1371/journal.pone.0254944 (e.g., the addition of 1, 2, or 3 prolines beyond the C-terminal “SS” of the VHH, such as a two-proline peptide). In some embodiments, the anti-VEGFA VHH (e.g., any of those described in the tables above) comprises a C-terminal modification of the addition of 1, 2 or 3 prolines to the C-terminus of the VHH (e.g., to the C-terminus of any of the VHHs described herein). In some embodiments, the anti-VEGFA VHH comprises a C-terminal modification of the addition of 2 prolines to the C-terminus of the VHH. In embodiments where two anti-VEGFA VHHs are linked in series (e.g., by a flexible peptide linker), in some instance only the C-terminal VHH will comprise the modification (e.g., only the C-terminal VHH comprises the sequence “PP” after the C-terminal “SS” of the VHH). In some instances, both VHHs linked in series will contain the modification (e.g., both will comprise the sequence “PP” after the C-terminal “SS” of each VHH).

In some embodiments, the anti-VEGFA binding domain of the immunocytokine composition comprises a light chain constant region. In some embodiments, the light chain constant region is one which contains one or more modifications which enhance the stability and/or manufacturability of the binding domain (or the immunocytokine composition as a whole). Such modifications are described in, for example, U.S. Pat. No. 9,777,067 and U.S. Patent Publication No. US20150239977A1 and include, for example, modifications of residue L154 of the light chain constant region (EU numbering), such as an L154K substitution and/or modifications of residue L201 (EU numbering), such as an L201K substitution. In some embodiments, the light chain constant region of the anti-VEGFA binding domain (e.g., a Fab) comprises an L154K substitution compared to the consensus sequence (e.g., as in SEQ ID NO: 277). In some embodiments, the light chain constant region of the anti-VEGFA binding domain (e.g., a Fab) comprises an L201K substitution compared to the consensus sequence. In some embodiments, the light chain constant region of the anti-VEGFA binding domain (e.g., a Fab) comprises L154K and L201K substitutions compared to the consensus sequence (e.g., as in SEQ ID NO: 278).

In some embodiments, an anti-VEGFA binding domain according to the instant disclosure can be a polypeptide which binds to VEGFA which is not derived from an antibody. In some embodiments, the anti-VEGFA binding domain comprises an anticalin which binds to VEGFA. In some embodiments, the anticalin comprises an amino acid sequence having at least 80%, 85%, 90% 95%, 96% 97%, 98%, 99%, or 100% identity to the sequence DGGGIRRSMSGTWYLKAMTVDREFPEMNLESVTPMTLTLLKGHNLEAKVTMLISGRCQ EVKAVLGRTKERKKYTADGGKHVAYIIPSAVRDHVIFYSEGQLHGKPVRGVKLVGRDP KNNLEALEDFEKAAGRLSTESILIPRQSETCSPG (SEQ ID NO: 226). In some embodiments, the anti-VEGFA binding domain comprises the sequence of SEQ ID NO: 226.

### Cytokines

Cytokines are proteins produced in the body that are important in cell signaling. Cytokines can modulate the immune system, and cytokine therapy utilizes the immunomodulatory properties of the molecules to enhance or regulate the immune system of a subject. Disclosed herein in some embodiments are immunocytokines compositions which comprise cytokines (e.g., modified cytokines and/or synthetic cytokines) linked in immunocytokine compositions which comprise anti-PD-1 and anti-VEGFA binding domains. In some embodiments, immunocytokine compositions of the instant disclosure can exhibit enhanced biological activity compared to individual cytokines by themselves or can modulate the immune system in advantageous ways difficult to achieve with individual cytokines.

A cytokine of an immunocytokine composition as provided herein can be any cytokine. Non-limiting examples of cytokines include interleukins (e.g., IL-2, IL-18, IL-7, IL-17), TNF family cytokines (e.g., TNFa, CD70, TNFSF14), interferons (e.g., IFNγ, IFNα. IFNβ), TGF-β family cytokines (e.g., TGFB1, TGFB2, TGFB3), chemokines (e.g., CCL2, CCL3, CXCL9, CXCL10) and others. In some embodiments, the cytokine of the immunocytokine composition is an interleukin. In some embodiments, the interleukin is selected from an IL-1 family cytokine (e.g., IL-18, IL-1β, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) and an IL-17 family cytokine (e.g., IL-17, IL-17F, IL-25). In some embodiments, the cytokine of the immunocytokine composition is selected from an IL-2 polypeptide, an IL-7 polypeptide, an IL-12 polypeptide, and an IL-18 polypeptide.

Cytokines of the immunocytokine compositions provided herein may be modified versions of the cytokines. In some embodiments, the cytokines comprise modifications (e.g., amino acid substitutions, additions, or deletions, attachment of polymers) which can modulate the activity of the cytokine (e.g., enhance activity, detune activity, or modulate the activity, such as by biasing the cytokine to one receptor or receptor subunit). In some embodiments, the cytokines comprise modifications in order to facilitate site specific attachment of a linker as provided herein. Additionally, cytokines provided herein may also be fused to additional polypeptides (e.g., antibody fusions, Fc fusions, etc.) or peptide sequences, such as artificial leader sequences, half-life extension sequences, or other peptides affixed to the N or C terminus of the cytokine. Cytokines may also be truncated versions of cytokines provided herein. In some embodiments, the cytokine comprises an amino acid sequence having at least 80%, 85%, 90%, 95%, 96%, 97%, 98%, 99%, or 100% identity to a naturally occurring cytokine (e.g., a human cytokine).

### IL-2 Polypeptides

The present disclosure describes in some embodiments anti-VEGFA binding domains and/or anti-PD-1 binding domains linked to interleukin-2 (IL-2) polypeptides as immunocytokine compositions and their use as human therapeutic agents. In some embodiments, the IL-2 polypeptide comprises an amino acid sequence having at least 80%, 85%, 90%, 95%, 96%, 97%, 98%, 99% or 100% a sequence identity to the sequence set forth in SEQ ID NO: 701. Unless otherwise specified, residue position numbering in reference to a modification to an IL-2 polypeptide described herein refers to SEQ ID NO: 701 as a reference sequence.

In some embodiments, the IL-2 polypeptide is biased towards one or more of the IL-2 receptor subunits relative to WT IL-2. In some embodiments, the IL-2 polypeptide retains binding to the IL-2 receptor alpha subunit but has detuned binding for the IL-2 receptor beta and gamma subunits. Non-limiting examples of such IL-2 polypeptides include those found in US20230303649A1, any one of which can be used as an IL-2 polypeptide in an immunocytokine composition as described herein (or can be suitably modified to be incorporated into an immunocytokine composition as described herein).

In other embodiments, the IL-2 polypeptide retains binding to the IL-2 receptor beta and/or gamma subunits (or the beta gamma heterodimer subunit) buy lacks or exhibits substantially diminished ability to bind to the IL-2 receptor alpha subunit. Non-limiting examples of such IL-2 polypeptides include those found in U.S. Ser. No. 11/633,488B2, any one of which can be used as an IL-2 polypeptide in an immunocytokine composition as described herein (or can be suitably modified to be incorporated into an immunocytokine composition as described herein).

In some embodiments, the IL-2 polypeptide comprises a modification which imparts favorable properties related to stability and/or expression of the IL-2 polypeptide but which otherwise generally does not substantially impact the activity of the IL-2 polypeptide. Several such modifications include, for example, a deletion of residue A1 of the IL-2 polypeptide, a T3A substitution, a C125S substitution, and/or a C125A substitution. Such modifications can generally be combined with other modifications described herein which can impact the activity of the IL-2 polypeptide described herein without causing detrimental effects. Thus, it is expressly contemplated within the instant disclosure that any IL-2 polypeptide described herein can further comprise a deletion of residue A1, a T3A substation, and/or a C125S or C125A substitution.

In some instances, it is desirable that the IL-2 polypeptide is one which is either activatable (e.g., has a null or low IL-2 related activity until activation, such as cleavage of a masking group by a tumor microenvironment protease) or one which is detuned (i.e., less potent/active) as compared to wild type IL-2 in order to provide a high degree of safety and low toxicity/side effects. Non-limiting examples of such activatable IL-2 polypeptides can be found in WO2024150175A1, any one of which can be used as an IL-2 polypeptide in an immunocytokine composition as described herein (or can be suitably modified to be incorporated into an immunocytokine composition as described herein). Additionally, the activation strategies described therein could also be applied to other IL-2 polypeptides, such as those described below which favor binding to the IL-2 receptor alpha subunit.

Alpha-Competent IL-2 Polypeptides with Detuned IL-2 Receptor Beta/Gamma Subunit Activity

In some embodiments, an IL-2 polypeptide of an immunocytokine composition described herein exhibits binding to and/or signaling through the IL-2 receptor alpha subunit (e.g., the IL-2 receptor αβγ complex). In some embodiments, the IL-2 polypeptide exhibits binding to and/or signaling through the IL-2 receptor alpha subunit which is comparable to or only slightly diminished compared to wild type IL-2. In some embodiments, the IL-2 polypeptide contains substantially detuned ability to bind to and/or signal through the IL-2 receptor beta and/or gamma subunits.

In some embodiments, the IL-2 polypeptide comprises one or more modification which reduces binding of the IL-2 polypeptide to the IL-2 receptor beta subunit.

In some embodiments, the IL-2 polypeptide comprises a substitution at one or more residues selected from H16, D20, D84, S87, N88, and V91. In some embodiments, the IL-2 polypeptide comprises one or more substitutions selected from H16S, D20V, D84K, S87A, N88D, N88R, V91A, and V91L. In some embodiments, the IL-2 polypeptide comprises an N88D or N88R substitution. In some embodiments, the IL-2 polypeptide comprises an amino acid substitution at residue N88. In some embodiments, the IL-2 polypeptide comprises an N88D substitution. In some embodiments, the IL-2 polypeptide comprises an N88R substitution.

In some embodiments, the IL-2 polypeptide comprises one or more modifications which reduce binding to the IL-2 receptor gamma subunit.

In some embodiments, the IL-2 polypeptide comprises a substitution at one or more residues selected from L12, E15, L19, Q22, T123, Q126, 1129, or S130. In some embodiments, the IL-2 polypeptide one or more substitution selected from L12A, L12V, L12Y, E15D, E15S, L19D, L19A, L19V, Q22T, T123A, Q126T, I129A, I129K, I129L or S130R. In some embodiments, the IL-2 polypeptide comprises a substitution at any one of residues L12, E15, L19, T123, Q126, or I129. In some embodiments, the IL-2 polypeptide comprises one or more substitutions selected from L12A, L12Y, E15D, E15S, L19A, L19D, T123A, Q126T, I129A, and I129K. In some embodiments, the IL-2 polypeptide comprises any one of the following sets of substitutions: Q126T; I129K; I129A, E15S, and T123A; E15D; L12A, L19A, and E15S; L12Y and L19D; L12A and L19A; or L19D. In some embodiments, the IL-2 polypeptide comprises an E15D or an L19D substitution. In some embodiments, the IL-2 polypeptide comprises an E15D substitution. In some embodiments, the IL-2 polypeptide comprises an L19D substitution.

In some embodiments, the IL-2 polypeptide comprises one or more modifications which favorably impact expression and/or stability of the IL-2 polypeptide.

In some embodiments, the IL-2 polypeptide comprises an A1 deletion, a T3A substitution, a C125S substitution, or a C125A substitution. In some embodiments, the IL-2 polypeptide comprises a T3A substitution, a C125S substitution, or a C125A substitution. In some embodiments, the IL-2 polypeptide comprises a T3A substitution. In some embodiments, the IL-2 polypeptide comprises a C125S substitution. In some embodiments, the IL-2 polypeptide a C125A substitution. In some embodiments, the IL-2 polypeptide comprises a T3A substitution and a C125A or C125S substitution. In some embodiments, the IL-2 polypeptide comprises a T3A substitution and a C125S substitution.

In some embodiments, the IL-2 exhibits reduced binding to heparin. In some embodiments, the IL-2 polypeptide comprises one or more modifications which reduce the binding of the IL-2 polypeptide to heparin. In some embodiments, reducing binding to heparin can favorably impact pharmacokinetic properties in vivo.

In some embodiments, the IL-2 polypeptide of comprises a modified B′C′ loop region of the IL-2 polypeptide. The B′C′ loop region refers to the amino acids which form the linkage between helixes B and C of IL-2 (e.g., human IL-2). The B′C′ loop region contains the amino acids positioned between amino acids 73 and 84 of wild type human IL-2 (SEQ ID NO: 701). In some embodiments, the modified B′C′ loop region of the IL-2 polypeptide comprises a deletion of one or more amino acids of the B′C′ loop region between amino acids 73 and 84 of the IL-2 polypeptide. In some embodiments, the modified B′C′ loop region of the IL-2 polypeptide and insertion of an exogenous peptide into the B′C′ loop region. In some embodiments, the modified B′C′ loop region of the IL-2 polypeptide comprises a deletion of one or more amino acids of the B′C′ loop region between amino acids 73 and 84 of the IL-2 polypeptide and an insertion of an exogenous peptide into the B′C′ loop region.

In some embodiments, the modified B′C′ loop region comprises a deletion of 1, 2, 3, 4, 5, 6, 7, 8, 9 or 10 amino acids between amino acids 73 and 84 of the IL-2 polypeptide. In some embodiments, the modified B′C′ loop region comprises a deletion of each amino acid 73 and 84 of the IL-2 polypeptide. In some embodiments, the modified B′C′ loop region comprises a deletion of each amino acid between 73 and 84 of the IL-2 polypeptide and insertion of an exogenous peptide.

In some embodiments, the modified B′C′ loop region comprises insertion of an exogenous peptide. In some embodiments, the exogenous peptide comprises the sequence GDGSIN (SEQ ID NO: 700). In some embodiments, the exogenous peptide consists of the sequence GDGSIN (SEQ ID NO: 700). In some embodiments, the modified B′C′ loop region comprises a deletion of each amino acid between amino acids 73 and 84 of the IL-2 polypeptide and an insertion of an exogenous peptide having the sequence GDGSIN (SEQ ID NO: 700) (i.e., the amino acids between 73 and 84 of the IL-2 polypeptide are replaced with the sequence GDGSIN (SEQ ID NO: 700)). In some embodiments, the IL-2 polypeptide comprises a peptide sequence having at least 80%, 85%, 90%, 95%, 96%, 97%, 98%, 99%, or 100% sequence identity to amino acids 1-72 of SEQ ID NO: 701 (i.e., the sequence APTSSSTKKTQLQLEHLLLDLQMILNGINNYKNPKLTRMLTFKFYMPKKATELKHLQCL EEELKPLEEVLNLA (SEQ ID NO: 784)) and a peptide sequence having at least 80%, 85%, 90%, 95%, 96%, 97%, 98%, 99%, or 100% sequence identity to amino acids 84-133 of SEQ ID NO: 701 (i.e., the sequence DLISNINVIVLELKGSETTFMCEYADETATIVEFLNRWITFCQSIISTLT (SEQ ID NO: 785). In some embodiments, the IL-2 polypeptide comprises a peptide sequence having at least 80%, 85%, 90%, 95%, 96%, 97%, 98%, 99%, or 100% identity to the sequence APTSSSTKKTQLQLEHLLLDLQMILNGINNYKNPKLTRMLTFKFYMPKKATELKHLQCL EEELKPLEEVLNLAGDGSINDLISNINVIVLELKGSETTFMCEYADETATIVEFLNRWITFC QSIISTLT (SEQ ID NO: 783). In some embodiments, the inserted exogenous peptide is QDASIH (SEQ ID NO: 786).

In some embodiments, the IL-2 polypeptide comprises a substitution at one or more amino acids selected from residue 32, residue 35, residue 38, residue 76, residue 81, or residue 83. In some embodiments, the IL-2 polypeptide comprises a K32S, K35E, R38A, K76A, R81S, or R83S substitution. In some embodiments, the IL-2 polypeptide comprises K32S. In some embodiments, the IL-2 polypeptide comprises K35E. In some embodiments, the IL-2 polypeptide comprises R38A. In some embodiments, the IL-2 polypeptide comprises K76A. In some embodiments, the IL-2 polypeptide comprises R81S. In some embodiments, the IL-2 polypeptide comprises R83S. In some embodiments, the IL-2 polypeptide comprises K32S, K35E, and R38A substitutions. In some embodiments, the IL-2 polypeptide comprises K76A and R81S substitutions. In some embodiments, the IL-2 polypeptide comprises K76A, R81S, and R81S substitutions.

In some embodiments, the IL-2 polypeptide is one which comprises an amino acid sequence having at least 80%, 85%, 90%, 95%, 96%, 97%, 98%, 99%, or 100% identity to any one of SEQ ID NOs: 704-774. In some embodiments, the IL-2 polypeptide is one which comprises an amino acid sequence having at least 80%, 85%, 90%, 95%, 96%, 97%, 98%, 99%, or 100% identity to any one of SEQ ID NOs: 704-774 and retains all of the substitutions relative to the IL-2 polypeptide of SEQ ID NO: 701. In some embodiments, the IL-2 polypeptide is one which comprises the amino acid sequence set forth in any one of SEQ ID NOs: 704-774. In some embodiments, the IL-2 polypeptide is one which comprises the amino acid sequence set forth in SEQ ID NO: 751. In some embodiments, the IL-2 polypeptide is one which comprises the amino acid sequence set forth in SEQ ID NO: 753. In some embodiments, the IL-2 polypeptide is one which comprises the amino acid sequence set forth in SEQ ID NO: 754. In some embodiments, the IL-2 polypeptide is one which comprises the amino acid sequence set forth in SEQ ID NO: 758.

In some embodiments, the IL-2 polypeptide comprises the following substitutions: T3A, F42A, Y45A, L72G, and C125A. In some embodiments, the IL-2 polypeptide comprises the following substitutions: T3A, Y31H, K35R, Q74P, N88D, and C125A. In some embodiments, the IL-2 polypeptide comprises the following substitutions: T3A, N88R, S130R, and IL15B′C′ loop. In some embodiments, the IL-2 polypeptide comprises the following substitutions: T3A, N88D, and C125S. In some embodiments, the IL-2 polypeptide comprises the following substitutions: T3A, N88R, and C125S. In some embodiments, the IL-2 polypeptide comprises the following substitutions: T3A, D20V, and C125S. In some embodiments, the IL-2 polypeptide comprises the following substitutions: T3A, H16S, N88D, and C125S. In some embodiments, the IL-2 polypeptide comprises the following substitutions: T3A, D20V, V91A, and C125S. In some embodiments, the IL-2 polypeptide comprises the following substitutions: T3A, E15S, N88D, and C125S. In some embodiments, the IL-2 polypeptide comprises the following substitutions: T3A, E15S, Q22T, V91L, and C125S. In some embodiments, the IL-2 polypeptide comprises the following substitutions: T3A, E15S, Q22T, N88D, and C125S. In some embodiments, the IL-2 polypeptide comprises the following substitutions: T3A, N88D, C125S, and Q126T. In some embodiments, the IL-2 polypeptide comprises the following substitutions: T3A, N88D, C125S, and I129A. In some embodiments, the IL-2 polypeptide comprises the following substitutions: T3A, N88D, C125S, and I129L. In some embodiments, the IL-2 polypeptide comprises the following substitutions: T3A, E15S, N88D, C125S, and I129A. In some embodiments, the IL-2 polypeptide comprises the following substitutions: T3A, E15S, N88D, C125S, and I129L. In some embodiments, the IL-2 polypeptide comprises the following substitutions: T3A, N88D, C125S, and I129K. In some embodiments, the IL-2 polypeptide comprises the following substitutions: T3A, E15S, H16S, N88D, and C125S. In some embodiments, the IL-2 polypeptide comprises the following substitutions: T3A, Q22T, N88D, E95S, and C125S. In some embodiments, the IL-2 polypeptide comprises the following substitutions: T3A, Q22T, S87A, N88D, and C125S. In some embodiments, the IL-2 polypeptide comprises the following substitutions: T3A, E15S, D84K, N88D, and C125S. In some embodiments, the IL-2 polypeptide comprises the following substitutions: T3A, E15S, Q22T, S87A, N88D, and C125S. In some embodiments, the IL-2 polypeptide comprises the following substitutions: T3A, E15S, N88D, C125S, and Q126T. In some embodiments, the IL-2 polypeptide comprises the following substitutions: T3A, N88R, C125S, and S130R. In some embodiments, the IL-2 polypeptide comprises the following substitutions: T3A, F42A, N88R, C125S, S130R, and IL15B′C′ loop. In some embodiments, the IL-2 polypeptide comprises the following substitutions: T3A, N88R, C125S, S130R, and GDGSIN BC′ loop. In some embodiments, the IL-2 polypeptide comprises the following substitutions: T3A, K76A, R81S, N88R, C125S, and S130R. In some embodiments, the IL-2 polypeptide comprises the following substitutions: T3A, K76A, R81S, R83S, N88R, C125S, and S130R. In some embodiments, the IL-2 polypeptide comprises the following substitutions: T3A, N88R, C125S, S130R, and QSGH AB loop. In some embodiments, the IL-2 polypeptide comprises the following substitutions: T3A, K32S, K35E, R38A, N88R, C125S, and S130R. In some embodiments, the IL-2 polypeptide comprises the following substitutions: T3A, N88R, I92L, C125S, and S130R. In some embodiments, the IL-2 polypeptide comprises the following substitutions: T3A, E15S, N88D, T123A, C125S, and I129A. In some embodiments, the IL-2 polypeptide comprises the following substitutions: T3A, N88D, T123A, and C125S. In some embodiments, the IL-2 polypeptide comprises the following substitutions: T3A, L12A, E15S, L19A, and C125S. In some embodiments, the IL-2 polypeptide comprises the following substitutions: T3A, L12A, E15S, L19D, and C125S. In some embodiments, the IL-2 polypeptide comprises the following substitutions: T3A, L12Y, E15S, L19A, and C125S. In some embodiments, the IL-2 polypeptide comprises the following substitutions: T3A, L12Y, L19D, and C125S. In some embodiments, the IL-2 polypeptide comprises the following substitutions: T3A, E15D, N88D, and C125S. In some embodiments, the IL-2 polypeptide comprises the following substitutions: T3A, L12A, E15S, L19A, N88D, and C125S. In some embodiments, the IL-2 polypeptide comprises the following substitutions: T3A, L12A, E15S, L19D, N88D, and C125S. In some embodiments, the IL-2 polypeptide comprises the following substitutions: T3A, L12Y, E15S, L19A, N88D, and C125S. In some embodiments, the IL-2 polypeptide comprises the following substitutions: T3A, L12Y, L19D, N88D, and C125S. In some embodiments, the IL-2 polypeptide comprises the following substitutions: T3A, L12V, L19V, N88D, and C125S. In some embodiments, the IL-2 polypeptide comprises the following substitutions: T3A, L12A, L19A, N88D, and C125S. In some embodiments, the IL-2 polypeptide comprises the following substitutions: T3A, L12Y, N88D, and C125S. In some embodiments, the IL-2 polypeptide comprises the following substitutions: T3A, L19D, N88D, and C125S. In some embodiments, the IL-2 polypeptide comprises the following substitutions: T3A, Y31H, K35R, Q74P, N88D, C125A, and Q126T. In some embodiments, the IL-2 polypeptide comprises the following substitutions: T3A, N88D, C125S, Q126T, and GDGSIN B′C′ loop. In some embodiments, the IL-2 polypeptide comprises the following substitutions: T3A, N88D, C125S, I129K, and GDGSIN BC′ loop. In some embodiments, the IL-2 polypeptide comprises the following substitutions: T3A, E15S, N88D, T123A, C125S, I129A, and GDGSIN B′C′ loop. In some embodiments, the IL-2 polypeptide comprises the following substitutions: T3A, E15D, N88D, C125S, and GDGSIN BC′ loop. In some embodiments, the IL-2 polypeptide comprises the following substitutions: T3A, L12A, E15S, L19A, N88D, C125S, and GDGSIN B′C′ loop. In some embodiments, the IL-2 polypeptide comprises the following substitutions: T3A, L12Y, L19D, N88D, C125S, and GDGSIN BC′ loop. In some embodiments, the IL-2 polypeptide comprises the following substitutions: T3A, L12A, L19A, N88D, C125S, and GDGSIN B′C′ loop. In some embodiments, the IL-2 polypeptide comprises the following substitutions: T3A, L19D, N88D, C125S, and GDGSIN BC′ loop. In some embodiments, the IL-2 polypeptide comprises the following substitutions: T3A, K76A, R81S, N88D, C125S, and Q126T. In some embodiments, the IL-2 polypeptide comprises the following substitutions: T3A, K76A, R81S, N88D, C125S, and I129K. In some embodiments, the IL-2 polypeptide comprises the following substitutions: T3A, E15S, K76A, R81S, N88D, T123A, C125S, and I129A. In some embodiments, the IL-2 polypeptide comprises the following substitutions: T3A, E15D, K76A, R81S, N88D, and C125S. In some embodiments, the IL-2 polypeptide comprises the following substitutions: T3A, L12A, E15S, L19A, K76A, R81S, N88D, and C125S. In some embodiments, the IL-2 polypeptide comprises the following substitutions: T3A, L12Y, L19D, K76A, R81S, N88D, and C125S. In some embodiments, the IL-2 polypeptide comprises the following substitutions: T3A, L12A, L19A, K76A, R81S, N88D, and C125S. In some embodiments, the IL-2 polypeptide comprises the following substitutions: T3A, L19D, K76A, R81S, N88D, and C125S. In some embodiments, the IL-2 polypeptide comprises the following substitutions: T3A, K32S, K35E, N88D, C125S, and Q126T. In some embodiments, the IL-2 polypeptide comprises the following substitutions: T3A, K32S, K35E, N88D, C125S, and I129K. In some embodiments, the IL-2 polypeptide comprises the following substitutions: T3A, E15S, K32S, K35E, N88D, T123A, C125S, and I129A. In some embodiments, the IL-2 polypeptide comprises the following substitutions: T3A, E15D, K32S, K35E, N88D, and C125S. In some embodiments, the IL-2 polypeptide comprises the following substitutions: T3A, L12A, E15S, L19A, K32S, K35E, N88D, and C125S. In some embodiments, the IL-2 polypeptide comprises the following substitutions: T3A, L12Y, L19D, K32S, K35E, N88D, and C125S. In some embodiments, the IL-2 polypeptide comprises the following substitutions: T3A, L12A, L19A, K32S, K35E, N88D, and C125S. In some embodiments, the IL-2 polypeptide comprises the following substitutions: T3A, L19D, K32S, K35E, N88D, and C125S.

Non-limiting examples of IL-2 polypeptides of the instant disclosure include those listed in Table 3 below. In Table 3 and the preceding paragraph, “IL15B′C′ loop” refers to a deletion of each amino acid between residues 73 and 84 of the IL-2 polypeptide (SEQ ID NO: 701 as a reference sequence) and insertion of the sequence GDASIH (SEQ ID NO: 786). In Table 3 and the preceding paragraph, “GDGSIN B′C′ loop” refers to a deletion of each amino acid between 73 and 84 of the IL-2 polypeptide (SEQ ID NO: 701 as a reference sequence) and insertion of the sequence GDGSIN (SEQ ID NO: 700). In Table 3 and the preceding paragraph, “QSGH AB loop” refers to a deletion of each amino acid between residues 28 and 39 (SEQ ID NO: 701 as a reference sequence) and insertion of the sequence QSGH (SEQ ID NO: 787).

TABLE 3
Exemplary IL-2 Polypeptides

| IL2 | | | |
|---|---|---|---|
| payload | | | SEQ |
| name | Substitutions | Sequence | ID NO |
| WT | | APTSSSTKKTQLQLEHLLLDLQMILNGINNYKNPK | 701 |
| IL-2 | | LTRMLTFKFYMPKKATELKHLQCLEEELKPLEEV | |
| | | LNLAQSKNFHLRPRDLISNINVIVLELKGSETTFMC | |
| | | EYADETATIVEFLNRWITFCQSIISTLT | |
| Aldesleukin | delA1, C125S | PTSSSTKKTQLQLEHLLLDLQMILNGINNYKNPKL | 702 |
| | | TRMLTFKFYMPKKATELKHLQCLEEELKPLEEVL | |
| | | NLAQSKNFHLRPRDLISNINVIVLELKGSETTFMCE | |
| | | YADETATIVEFLNRWITFSQSIISTLT | |
| 2P1 | IL2_T3A_F42A_ | APASSSTKKTQLQLEHLLLDLQMILNGINNYKNPK | 704 |
| | Y45A_L72G_ | LTRMLTAKFAMPKKATELKHLQCLEEELKPLEEV | |
| | C125A | LNGAQSKNFHLRPRDLISNINVIVLELKGSETTFMC | |
| | | EYADETATIVEFLNRWITFAQSIISTLT | |
| 2P2 | IL2_T3A_Y31H_ | APASSSTKKTQLQLEHLLLDLQMILNGINNHKNPR | 705 |
| | K35R_Q74P_ | LTRMLTFKFYMPKKATELKHLQCLEEELKPLEEV | |
| | N88D_C125A | LNLAPSKNFHLRPRDLISDINVIVLELKGSETTFMC | |
| | | EYADETATIVEFLNRWITFAQSIISTLT | |
| 2P3 | IL2_T3A_N88R_ | APASSSTKKTQLQLEHLLLDLQMILNGINNYKNPK | 706 |
| | S130R_IL15B′C′ | LTRMLTFKFYMPKKATELKHLQCLEEELKPLEEV | |
| | loop | LNLAGDASIHDLISRINVIVLELKGSETTFMCEYAD | |
| | | ETATIVEFLNRWITFSQSIIRTLT | |
| 2P4 | IL2_T3A_N88D_ | APASSSTKKTQLQLEHLLLDLQMILNGINNYKNPK | 707 |
| | C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P5 | IL2_T3A_N88R_ | APASSSTKKTQLQLEHLLLDLQMILNGINNYKNPK | 708 |
| | C125S | LTRMLTFKFYMPKKATELKHLQCLEEELKPLEEV | |
| | | LNLAQSKNFHLRPRDLISRINVIVLELKGSETTFMC | |
| | | EYADETATIVEFLNRWITFSQSIISTLT | |
| 2P6 | IL2_T3A_D20V_ | APASSSTKKTQLQLEHLLLVLQMILNGINNYKNPK | 709 |
| | C125S | LTRMLTFKFYMPKKATELKHLQCLEEELKPLEEV | |
| | | LNLAQSKNFHLRPRDLISNINVIVLELKGSETTFMC | |
| | | EYADETATIVEFLNRWITFSQSIISTLT | |
| 2P7 | IL2_T3A_H16S_ | APASSSTKKTQLQLESLLLDLQMILNGINNYKNPK | 710 |
| | N88D_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P8 | IL2_T3A_D20V_ | APASSSTKKTQLQLEHLLLVLQMILNGINNYKNPK | 711 |
| | V91A_C125S | LTRMLTFKFYMPKKATELKHLQCLEEELKPLEEV | |
| | | LNLAQSKNFHLRPRDLISNINAIVLELKGSETTFMC | |
| | | EYADETATIVEFLNRWITFSQSIISTLT | |
| 2P9 | IL2_T3A_E15S_ | APASSSTKKTQLQLSHLLLDLQMILNGINNYKNPK | 712 |
| | N88D_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P10 | IL2_T3A_E15S_ | APASSSTKKTQLQLSHLLLDLTMILNGINNYKNPK | 713 |
| | Q22T_V91L_ | LTRMLTFKFYMPKKATELKHLQCLEEELKPLEEV | |
| | C125S | LNLAQSKNFHLRPRDLISNINLIVLELKGSETTFMC | |
| | | EYADETATIVEFLNRWITFSQSIISTLT | |
| 2P11 | IL2_T3A_E15S_ | APASSSTKKTQLQLSHLLLDLTMILNGINNYKNPK | 714 |
| | Q22T_N88D_ | LTRMLTFKFYMPKKATELKHLQCLEEELKPLEEV | |
| | C125S | LNLAQSKNFHLRPRDLISDINVIVLELKGSETTFMC | |
| | | EYADETATIVEFLNRWITFSQSIISTLT | |
| 2P12 | IL2_T3A_N88D_ | APASSSTKKTQLQLEHLLLDLQMILNGINNYKNPK | 715 |
| | C125S_Q126T | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSTSIISTLT | |
| 2P13 | IL2_T3A_N88D_ | APASSSTKKTQLQLEHLLLDLQMILNGINNYKNPK | 716 |
| | C125S_I129A | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIASTLT | |
| 2P14 | IL2_T3A_N88D_ | APASSSTKKTQLQLEHLLLDLQMILNGINNYKNPK | 717 |
| | C125S_I129L | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSILSTLT | |
| 2P15 | IL2_T3A_E15S_ | APASSSTKKTQLQLSHLLLDLQMILNGINNYKNPK | 718 |
| | N88D_C125S_ | LTRMLTFKFYMPKKATELKHLQCLEEELKPLEEV | |
| | I129A | LNLAQSKNFHLRPRDLISDINVIVLELKGSETTFMC | |
| | | EYADETATIVEFLNRWITFSQSIASTLT | |
| 2P16 | IL2_T3A_E15S_ | APASSSTKKTQLQLSHLLLDLQMILNGINNYKNPK | 719 |
| | N88D_C125S_ | LTRMLTFKFYMPKKATELKHLQCLEEELKPLEEV | |
| | I129L | LNLAQSKNFHLRPRDLISDINVIVLELKGSETTFMC | |
| | | EYADETATIVEFLNRWITFSQSILSTLT | |
| 2P17 | IL2_T3A_N88D_ | APASSSTKKTQLQLEHLLLDLQMILNGINNYKNPK | 720 |
| | C125S_I129K | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIKSTLT | |
| 2P18 | IL2_T3A_E15S_ | APASSSTKKTQLQLSSLLLDLQMILNGINNYKNPK | 721 |
| | H16S_N88D_ | LTRMLTFKFYMPKKATELKHLQCLEEELKPLEEV | |
| | C125S | LNLAQSKNFHLRPRDLISDINVIVLELKGSETTFMC | |
| | | EYADETATIVEFLNRWITFSQSIISTLT | |
| 2P19 | IL2_T3A_Q22T_ | APASSSTKKTQLQLEHLLLDLTMILNGINNYKNPK | 722 |
| | N88D_E95S_ | LTRMLTFKFYMPKKATELKHLQCLEEELKPLEEV | |
| | C125S | LNLAQSKNFHLRPRDLISDINVIVLSLKGSETTFMC | |
| | | EYADETATIVEFLNRWITFSQSIISTLT | |
| 2P20 | IL2_T3A_Q22T_ | APASSSTKKTQLQLEHLLLDLTMILNGINNYKNPK | 723 |
| | S87A_N88D_ | LTRMLTFKFYMPKKATELKHLQCLEEELKPLEEV | |
| | C125S | LNLAQSKNFHLRPRDLIADINVIVLELKGSETTFMC | |
| | | EYADETATIVEFLNRWITFSQSIISTLT | |
| 2P21 | IL2_T3A_E15S_ | APASSSTKKTQLQLSHLLLDLQMILNGINNYKNPK | 724 |
| | D84K_N88D_ | LTRMLTFKFYMPKKATELKHLQCLEEELKPLEEV | |
| | C125S | LNLAQSKNFHLRPRKLISDINVIVLELKGSETTFMC | |
| | | EYADETATIVEFLNRWITFSQSIISTLT | |
| 2P22 | IL2_T3A_E15S_ | APASSSTKKTQLQLSHLLLDLTMILNGINNYKNPK | 725 |
| | Q22T_S87A_ | LTRMLTFKFYMPKKATELKHLQCLEEELKPLEEV | |
| | N88D_C125S | LNLAQSKNFHLRPRDLIADINVIVLELKGSETTFMC | |
| | | EYADETATIVEFLNRWITFSQSIISTLT | |
| 2P23 | IL2_T3A_E15S_ | APASSSTKKTQLQLSHLLLDLQMILNGINNYKNPK | 726 |
| | N88D_C125S_ | LTRMLTFKFYMPKKATELKHLQCLEEELKPLEEV | |
| | Q126T | LNLAQSKNFHLRPRDLISDINVIVLELKGSETTFMC | |
| | | EYADETATIVEFLNRWITFSTSIISTLT | |
| 2P24 | IL2_T3A_N88R_ | APASSSTKKTQLQLEHLLLDLQMILNGINNYKNPK | 727 |
| | C125S_S130R | LTRMLTFKFYMPKKATELKHLQCLEEELKPLEEV | |
| | | LNLAQSKNFHLRPRDLISRINVIVLELKGSETTFMC | |
| | | EYADETATIVEFLNRWITFSQSIIRTLT | |
| 2P25 | IL2_T3A_F42A | APASSSTKKTQLQLEHLLLDLQMILNGINNYKNPK | 728 |
| | N88R_C125S_ | LTRMLTAKFYMPKKATELKHLQCLEEELKPLEEV | |
| | S130R_IL15B′C′ | LNLAGDASIHDLISRINVIVLELKGSETTFMCEYAD | |
| | loop | ETATIVEFLNRWITFSQSIIRTLT | |
| 2P26 | IL2_T3A_N88R_ | APASSSTKKTQLQLEHLLLDLQMILNGINNYKNPK | 729 |
| | C125S_S130R_ | LTRMLTFKFYMPKKATELKHLQCLEEELKPLEEV | |
| | GDGSIN B′C′ | LNLAGDGSINDLISRINVIVLELKGSETTFMCEYAD | |
| | loop | ETATIVEFLNRWITFSQSIIRTLT | |
| 2P27 | IL2_T3A_K76A_ | APASSSTKKTQLQLEHLLLDLQMILNGINNYKNPK | 730 |
| | R81S_N88R_ | LTRMLTFKFYMPKKATELKHLQCLEEELKPLEEV | |
| | C125S_S130R | LNLAQSANFHLSPRDLISRINVIVLELKGSETTFMC | |
| | | EYADETATIVEFLNRWITFSQSIIRTLT | |
| 2P28 | IL2_T3A_K76A_ | APASSSTKKTQLQLEHLLLDLQMILNGINNYKNPK | 731 |
| | R81S_R83S_ | LTRMLTFKFYMPKKATELKHLQCLEEELKPLEEV | |
| | N88R_C125S_ | LNLAQSANFHLSPSDLISRINVIVLELKGSETTFMC | |
| | S130R | EYADETATIVEFLNRWITFSQSIIRTLT | |
| 2P29 | IL2_T3A_N88R_ | APASSSTKKTQLQLEHLLLDLQMILNGIQSGHMLT | 732 |
| | C125S_S130R_ | FKFYMPKKATELKHLQCLEEELKPLEEVLNLAQS | |
| | QSGH AB loop | KNFHLRPRDLISRINVIVLELKGSETTFMCEYADET | |
| | | ATIVEFLNRWITFSQSIIRTLT | |
| 2P30 | IL2_T3A_K32S_ | APASSSTKKTQLQLEHLLLDLQMILNGINNYSNPE | 733 |
| | K35E_R38A_ | LTAMLTFKFYMPKKATELKHLQCLEEELKPLEEV | |
| | N88R_C125S_ | LNLAQSKNFHLRPRDLISRINVIVLELKGSETTFMC | |
| | S130R | EYADETATIVEFLNRWITFSQSIIRTLT | |
| 2P31 | IL2_T3A_N88R_ | APASSSTKKTQLQLEHLLLDLQMILNGINNYKNPK | 734 |
| | I92L_C125S_ | LTRMLTFKFYMPKKATELKHLQCLEEELKPLEEV | |
| | S130R | LNLAQSKNFHLRPRDLISRINVLVLELKGSETTFM | |
| | | CEYADETATIVEFLNRWITFSQSIIRTLT | |
| 2P32 | IL2_T3A_E15S_ | APASSSTKKTQLQLSHLLLDLQMILNGINNYKNPK | 735 |
| | N88D_T123A_ | LTRMLTFKFYMPKKATELKHLQCLEEELKPLEEV | |
| | C125S_I129A | LNLAQSKNFHLRPRDLISDINVIVLELKGSETTFMC | |
| | | EYADETATIVEFLNRWIAFSQSIASTLT | |
| 2P33 | IL2_T3A_N88D_ | APASSSTKKTQLQLEHLLLDLQMILNGINNYKNPK | 736 |
| | T123A_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWIAFSQSIISTLT | |
| 2P34 | IL2_T3A_L12A_ | APASSSTKKTQAQLSHLLADLQMILNGINNYKNPK | 737 |
| | E15S_L19A_ | LTRMLTFKFYMPKKATELKHLQCLEEELKPLEEV | |
| | C125S | LNLAQSKNFHLRPRDLISNINVIVLELKGSETTFMC | |
| | | EYADETATIVEFLNRWITFSQSIISTLT | |
| 2P35 | IL2_T3A_L12A_ | APASSSTKKTQAQLSHLLDDLQMILNGINNYKNPK | 738 |
| | E15S_L19D_ | LTRMLTFKFYMPKKATELKHLQCLEEELKPLEEV | |
| | C125S | LNLAQSKNFHLRPRDLISNINVIVLELKGSETTFMC | |
| | | EYADETATIVEFLNRWITFSQSIISTLT | |
| 2P36 | IL2_T3A_L12Y_ | APASSSTKKTQYQLSHLLADLQMILNGINNYKNPK | 739 |
| | E15S_L19A_ | LTRMLTFKFYMPKKATELKHLQCLEEELKPLEEV | |
| | C125S | LNLAQSKNFHLRPRDLISNINVIVLELKGSETTFMC | |
| | | EYADETATIVEFLNRWITFSQSIISTLT | |
| 2P37 | IL2_T3A_L12Y_ | APASSSTKKTQYQLEHLLDDLQMILNGINNYKNP | 740 |
| | L19D_C125S | KLTRMLTFKFYMPKKATELKHLQCLEEELKPLEE | |
| | | VLNLAQSKNFHLRPRDLISNINVIVLELKGSETTFM | |
| | | CEYADETATIVEFLNRWITFSQSIISTLT | |
| 2P38 | IL2_T3A_E15D_ | APASSSTKKTQLQLDHLLLDLQMILNGINNYKNPK | 741 |
| | N88D_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P39 | IL2_T3A_L12A_ | APASSSTKKTQAQLSHLLADLQMILNGINNYKNPK | 742 |
| | E15S_L19A_ | LTRMLTFKFYMPKKATELKHLQCLEEELKPLEEV | |
| | N88D_C125S | LNLAQSKNFHLRPRDLISDINVIVLELKGSETTFMC | |
| | | EYADETATIVEFLNRWITFSQSIISTLT | |
| 2P40 | IL2_T3A_L12A_ | APASSSTKKTQAQLSHLLDDLQMILNGINNYKNPK | 743 |
| | E15S_L19D_ | LTRMLTFKFYMPKKATELKHLQCLEEELKPLEEV | |
| | N88D_C125S | LNLAQSKNFHLRPRDLISDINVIVLELKGSETTFMC | |
| | | EYADETATIVEFLNRWITFSQSIISTLT | |
| 2P41 | IL2_T3A_L12Y_ | APASSSTKKTQYQLSHLLADLQMILNGINNYKNPK | 744 |
| | E15S_L19A_ | LTRMLTFKFYMPKKATELKHLQCLEEELKPLEEV | |
| | N88D_C125S | LNLAQSKNFHLRPRDLISDINVIVLELKGSETTFMC | |
| | | EYADETATIVEFLNRWITFSQSIISTLT | |
| 2P42 | IL2_T3A_L12Y_ | APASSSTKKTQYQLEHLLDDLQMILNGINNYKNP | 745 |
| | L19D_N88D_ | KLTRMLTFKFYMPKKATELKHLQCLEEELKPLEE | |
| | C125S | VLNLAQSKNFHLRPRDLISDINVIVLELKGSETTFM | |
| | | CEYADETATIVEFLNRWITFSQSIISTLT | |
| 2P43 | IL2_T3A_L12V_ | APASSSTKKTQVQLEHLLVDLQMILNGINNYKNP | 746 |
| | L19V_N88D_ | KLTRMLTFKFYMPKKATELKHLQCLEEELKPLEE | |
| | C125S | VLNLAQSKNFHLRPRDLISDINVIVLELKGSETTFM | |
| | | CEYADETATIVEFLNRWITFSQSIISTLT | |
| 2P44 | IL2_T3A_L12A_ | APASSSTKKTQAQLEHLLADLQMILNGINNYKNP | 747 |
| | L19A_N88D_ | KLTRMLTFKFYMPKKATELKHLQCLEEELKPLEE | |
| | C125S | VLNLAQSKNFHLRPRDLISDINVIVLELKGSETTFM | |
| | | CEYADETATIVEFLNRWITFSQSIISTLT | |
| 2P45 | IL2_T3A_L12Y_ | APASSSTKKTQYQLEHLLLDLQMILNGINNYKNPK | 748 |
| | N88D_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P46 | IL2_T3A_L19D_ | APASSSTKKTQLQLEHLLDDLQMILNGINNYKNPK | 749 |
| | N88D_C125S | LTRMLTFKFYMPKKATELKHLQCLEEELKPLEEV | |
| | | LNLAQSKNFHLRPRDLISDINVIVLELKGSETTFMC | |
| | | EYADETATIVEFLNRWITFSQSIISTLT | |
| 2P47 | IL2_T3A_Y31H_ | APASSSTKKTQLQLEHLLLDLQMILNGINNHKNPR | 750 |
| | K35R_Q74P_ | LTRMLTFKFYMPKKATELKHLQCLEEELKPLEEV | |
| | N88D_C125A_ | LNLAPSKNFHLRPRDLISDINVIVLELKGSETTFMC | |
| | Q126T | EYADETATIVEFLNRWITFATSIISTLT | |
| 2P48 | IL2_T3A_N88D_ | APASSSTKKTQLQLEHLLLDLQMILNGINNYKNPK | 751 |
| | C125S_Q126T_ | LTRMLTFKFYMPKKATELKHLQCLEEELKPLEEV | |
| | GDGSIN B′C′ | LNLAGDGSINDLISDINVIVLELKGSETTFMCEYAD | |
| | loop | ETATIVEFLNRWITFSTSIISTLT | |
| 2P49 | IL2_T3A_N88D_ | APASSSTKKTQLQLEHLLLDLQMILNGINNYKNPK | 752 |
| | C125S_1129K_ | LTRMLTFKFYMPKKATELKHLQCLEEELKPLEEV | |
| | GDGSIN B′C′ | LNLAGDGSINDLISDINVIVLELKGSETTFMCEYAD | |
| | loop | ETATIVEFLNRWITFSQSIKSTLT | |
| 2P50 | IL2_T3A_E15S_ | APASSSTKKTQLQLSHLLLDLQMILNGINNYKNPK | 753 |
| | N88D_T123A_ | LTRMLTFKFYMPKKATELKHLQCLEEELKPLEEV | |
| | C125S_I129A_ | LNLAGDGSINDLISDINVIVLELKGSETTFMCEYAD | |
| | GDGSIN B′C′ | ETATIVEFLNRWIAFSQSIASTLT | |
| | loop | | |
| 2P51 | IL2_T3A_E15D_ | APASSSTKKTQLQLDHLLLDLQMILNGINNYKNPK | 754 |
| | N88D_C125S_ | LTRMLTFKFYMPKKATELKHLQCLEEELKPLEEV | |
| | GDGSIN B′C′ | LNLAGDGSINDLISDINVIVLELKGSETTFMCEYAD | |
| | loop | ETATIVEFLNRWITFSQSIISTLT | |
| 2P52 | IL2_T3A_L12A_ | APASSSTKKTQAQLSHLLADLQMILNGINNYKNPK | 755 |
| | E15S_L19A_ | LTRMLTFKFYMPKKATELKHLQCLEEELKPLEEV | |
| | N88D_C125S_ | LNLAGDGSINDLISDINVIVLELKGSETTFMCEYAD | |
| | GDGSIN B′C′ | ETATIVEFLNRWITFSQSIISTLT | |
| | loop | | |
| 2P53 | IL2_T3A_L12Y_ | APASSSTKKTQYQLEHLLDDLQMILNGINNYKNP | 756 |
| | L19D_N88D_ | KLTRMLTFKFYMPKKATELKHLQCLEEELKPLEE | |
| | C125S GDGSIN | VLNLAGDGSINDLISDINVIVLELKGSETTFMCEYA | |
| | B′C′ loop | DETATIVEFLNRWITFSQSIISTLT | |
| 2P54 | IL2_T3A_L12A_ | APASSSTKKTQAQLEHLLADLQMILNGINNYKNP | 757 |
| | L19A_N88D_ | KLTRMLTFKFYMPKKATELKHLQCLEEELKPLEE | |
| | C125S GDGSIN | VLNLAGDGSINDLISDINVIVLELKGSETTFMCEYA | |
| | B′C′ loop | DETATIVEFLNRWITFSQSIISTLT | |
| 2P55 | IL2_T3A_L19D_ | APASSSTKKTQLQLEHLLDDLQMILNGINNYKNPK | 758 |
| | N88D_C125S_ | LTRMLTFKFYMPKKATELKHLQCLEEELKPLEEV | |
| | GDGSIN B′C′ | LNLAGDGSINDLISDINVIVLELKGSETTFMCEYAD | |
| | loop | ETATIVEFLNRWITFSQSIISTLT | |
| 2P56 | IL2_T3A_K76A_ | APASSSTKKTQLQLEHLLLDLQMILNGINNYKNPK | 759 |
| | R81S_N88D_ | LTRMLTFKFYMPKKATELKHLQCLEEELKPLEEV | |
| | C125S_Q126T | LNLAQSANFHLSPRDLISDINVIVLELKGSETTFMC | |
| | | EYADETATIVEFLNRWITFSTSIISTLT | |
| 2P57 | IL2_T3A_K76A_ | APASSSTKKTQLQLEHLLLDLQMILNGINNYKNPK | 760 |
| | R81S_N88D_ | LTRMLTFKFYMPKKATELKHLQCLEEELKPLEEV | |
| | 25S_I129K | LNLAQSANFHLSPRDLISDINVIVLELKGSETTFMC | |
| | C1 | EYADETATIVEFLNRWITFSQSIKSTLT | |
| 2P58 | IL2_T3A_E15S_ | APASSSTKKTQLQLSHLLLDLQMILNGINNYKNPK | 761 |
| | K76A_R81S_ | LTRMLTFKFYMPKKATELKHLQCLEEELKPLEEV | |
| | N88D_T123A_ | LNLAQSANFHLSPRDLISDINVIVLELKGSETTFMC | |
| | C125S_I129A | EYADETATIVEFLNRWIAFSQSIASTLT | |
| 2P59 | IL2_T3A_E15D_ | APASSSTKKTQLQLDHLLLDLQMILNGINNYKNPK | 762 |
| | K76A_R81S_ | LTRMLTFKFYMPKKATELKHLQCLEEELKPLEEV | |
| | N88D_C125S | LNLAQSANFHLSPRDLISDINVIVLELKGSETTFMC | |
| | | EYADETATIVEFLNRWITFSQSIISTLT | |
| 2P60 | IL2_T3A_L12A_ | APASSSTKKTQAQLSHLLADLQMILNGINNYKNPK | 763 |
| | E15S_L19A_ | LTRMLTFKFYMPKKATELKHLQCLEEELKPLEEV | |
| | K76A_R81S_ | LNLAQSANFHLSPRDLISDINVIVLELKGSETTFMC | |
| | N88D_C125S | EYADETATIVEFLNRWITFSQSIISTLT | |
| 2P61 | IL2_T3A_L12Y_ | APASSSTKKTQYQLEHLLDDLQMILNGINNYKNP | 764 |
| | L19D_K76A_ | KLTRMLTFKFYMPKKATELKHLQCLEEELKPLEE | |
| | R81S_N88D_ | VLNLAQSANFHLSPRDLISDINVIVLELKGSETTFM | |
| | C125S | CEYADETATIVEFLNRWITFSQSIISTLT | |
| 2P62 | IL2_T3A_L12A_ | APASSSTKKTQAQLEHLLADLQMILNGINNYKNP | 765 |
| | L19A_K76A_ | KLTRMLTFKFYMPKKATELKHLQCLEEELKPLEE | |
| | R81S_N88D_ | VLNLAQSANFHLSPRDLISDINVIVLELKGSETTFM | |
| | C125S | CEYADETATIVEFLNRWITFSQSIISTLT | |
| 2P63 | IL2_T3A_L19D_ | APASSSTKKTQLQLEHLLDDLQMILNGINNYKNPK | 766 |
| | K76A_R81S_ | LTRMLTFKFYMPKKATELKHLQCLEEELKPLEEV | |
| | N88D_C125S | LNLAQSANFHLSPRDLISDINVIVLELKGSETTFMC | |
| | | EYADETATIVEFLNRWITFSQSIISTLT | |
| 2P64 | IL2_T3A_K32S_ | APASSSTKKTQLQLEHLLLDLQMILNGINNYSNPE | 767 |
| | K35E_N88D_ | LTRMLTFKFYMPKKATELKHLQCLEEELKPLEEV | |
| | C125S_Q126T | LNLAQSKNFHLRPRDLISDINVIVLELKGSETTFMC | |
| | | EYADETATIVEFLNRWITFSTSIISTLT | |
| 2P65 | IL2_T3A_K32S_ | APASSSTKKTQLQLEHLLLDLQMILNGINNYSNPE | 768 |
| | K35E_N88D_ | LTRMLTFKFYMPKKATELKHLQCLEEELKPLEEV | |
| | C125S_I129K | LNLAQSKNFHLRPRDLISDINVIVLELKGSETTFMC | |
| | | EYADETATIVEFLNRWITFSQSIKSTLT | |
| 2P66 | IL2_T3A_E15S_ | APASSSTKKTQLQLSHLLLDLQMILNGINNYSNPE | 769 |
| | K32S_K35E_ | LTRMLTFKFYMPKKATELKHLQCLEEELKPLEEV | |
| | N88D_T123A_ | LNLAQSKNFHLRPRDLISDINVIVLELKGSETTFMC | |
| | C125S_I129A | EYADETATIVEFLNRWIAFSQSIASTLT | |
| 2P67 | IL2_T3A_E15D_ | APASSSTKKTQLQLDHLLLDLQMILNGINNYSNPE | 770 |
| | K32S_K35E_ | LTRMLTFKFYMPKKATELKHLQCLEEELKPLEEV | |
| | N88D_C125S | LNLAQSKNFHLRPRDLISDINVIVLELKGSETTFMC | |
| | | EYADETATIVEFLNRWITFSQSIISTLT | |
| 2P68 | IL2_T3A_L12A_ | APASSSTKKTQAQLSHLLADLQMILNGINNYSNPE | 771 |
| | E15S_L19A_ | LTRMLTFKFYMPKKATELKHLQCLEEELKPLEEV | |
| | K32S_K35E_ | LNLAQSKNFHLRPRDLISDINVIVLELKGSETTFMC | |
| | N88D_C125S | EYADETATIVEFLNRWITFSQSIISTLT | |
| 2P69 | IL2_T3A_L12Y_ | APASSSTKKTQYQLEHLLDDLQMILNGINNYSNPE | 772 |
| | L19D_K32S_ | LTRMLTFKFYMPKKATELKHLQCLEEELKPLEEV | |
| | K35E_N88D_ | LNLAQSKNFHLRPRDLISDINVIVLELKGSETTFMC | |
| | C125S | EYADETATIVEFLNRWITFSQSIISTLT | |
| 2P70 | IL2_T3A_L12A_ | APASSSTKKTQAQLEHLLADLQMILNGINNYSNPE | 773 |
| | L19A_K32S_ | LTRMLTFKFYMPKKATELKHLQCLEEELKPLEEV | |
| | K35E_N88D_ | LNLAQSKNFHLRPRDLISDINVIVLELKGSETTFMC | |
| | C125S | EYADETATIVEFLNRWITFSQSIISTLT | |
| 2P71 | IL2_T3A_L19D_ | APASSSTKKTQLQLEHLLDDLQMILNGINNYSNPE | 774 |
| | K32S_K35E_ | LTRMLTFKFYMPKKATELKHLQCLEEELKPLEEV | |
| | N88D_C125S | LNLAQSKNFHLRPRDLISDINVIVLELKGSETTFMC | |
| | | EYADETATIVEFLNRWITFSQSIISTLT | |

Beta-Gamma Competent IL-2 Polypeptides with Detuned IL-2 Receptor Alpha Binding

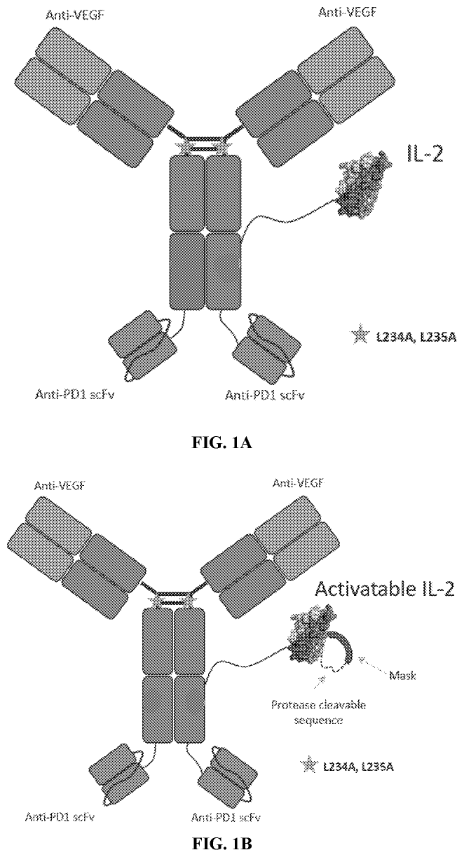

In some embodiments, an IL-2 polypeptide of an immunocytokine composition described herein is biased in favor of binding to and/or signaling through the IL-2 receptor beta subunit, IL-2 receptor gamma subunit, or the IL-2 receptor beta/gamma complex. In some embodiments, such IL-2 polypeptides exhibits substantially reduced binding to 11-2 receptor alpha subunit or the receptor alpha/beta/gamma complex. Non-limiting examples of such 11-2 polypeptides are described in, for example, U.S. Ser. No. 11/633,488B2, the contents of which are incorporated by reference. In some embodiments, an 11-2 polypeptide described therein is preferred for use in the immunocytokine composition of the instant disclosure (e.g., Composition A2 shown in FIG. 1H therein).

In some preferred embodiments of immunocytokine compositions which contain IL-2 polypeptides which exhibit substantially no ability to bind the IL-2 receptor alpha subunit yet retain binding and signaling ability through the beta and/or gamma subunit, the IL-2 polypeptide comprises polymers (e.g., PEG polymers) attached at one or both of residues 42 and 45 of the IL-2 polypeptide (e.g., F42Y and Y45). In some embodiments, one of the polymers forms a part of the linker which attaches the IL-2 polypeptide to the immunocytokine composition (e.g., the polymer attached at residue F42Y contains a conjugation handle such as an azide). In some embodiments, such an IL-2 polypeptide is synthetic (e.g., synthesized via KAHA ligation and further containing Hse41, Hse71, Hse104, Nle23, Nle39, and Nle 46). In some embodiments, the IL-2 polypeptide comprises a C125S substitution. In some embodiments, the polymers (E.g., the PEG polymers) attached at these residues 42 and 45 have a molecular weight of about 200-1000 Daltons. In some embodiments, such an IL-2 polypeptide comprises SEQ ID NO: 703.

Additional IL-2 polypeptides with similar properties are also contemplated as within the scope of the instant disclosure. Modifications to such IL-2 polypeptides encompass mutations, addition of various functionalities, deletion of amino acids, addition of amino acids, or any other alteration of the wild-type version of the protein or protein fragment. Functionalities which may be added to polypeptides include polymers, linkers, alkyl groups, detectable molecules such as chromophores or fluorophores, reactive functional groups, or any combination thereof. In some embodiments, functionalities are added to individual amino acids of the polypeptides. In some embodiments, functionalities are added site-specifically to the polypeptides.

In some embodiments, the IL-2 polypeptide of the comprises one or more modifications in addition to a modification needed to attach the linker to the relevant residue of the IL-2 polypeptide (e.g., an amino acid substitution at a residue to which the linker is not attached).

In some embodiments, the IL-2 polypeptide of the immunocytokine composition described herein contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues.

In some embodiments, the IL-2 polypeptide of the immunocytokine composition comprises an amino acid sequence having at least 80%, 85%, 90%, 91%, 92%, 93%, 94%, 95%, 96%, 97%, 98%, or 99% sequence identity to the sequence set forth in SEQ ID NO: 701.

In some embodiments, the IL-2 polypeptide comprises an amino acid sequence of SEQ ID NO: 703. In some embodiments, the IL-2 polypeptide comprises an amino acid sequence at least 85% identical to the sequence of SEQ ID NO: 703. In some embodiments, the IL-2 polypeptide comprises an amino acid sequence at least 80%, 85%, 90%, 95%, 96%, 97%, 98%, 99%, or 100% identical to the sequence of SEQ ID NO: 703.

In some embodiments, the IL-2 polypeptide of the immunocytokine composition described herein comprises at least 1, at least 2, at least 3, at least 4, at least 5, at least 6, at least 7, or at least 9 amino acid substitutions, wherein the amino acid substitutions are relative to SEQ ID NO: 701. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

In some embodiments, the IL-2 polypeptide comprising of the immunocytokine composition described herein comprises at least 1, at least 2, at least 3, at least 4, at least 5, at least 6, at least 7, or at least 9 natural amino acid substitutions, wherein the natural amino acid substitutions are relative to SEQ ID NO: 701. In some embodiments, the IL-2 polypeptide comprises 1 to 9 natural amino acid substitutions. In some embodiments, the IL-2 polypeptide comprises 1 or 2 natural amino acid substitutions, 1 to 3 natural amino acid substitutions, 1 to 4 natural amino acid substitutions, 1 to 5 natural amino acid substitutions, 1 to 6 natural amino acid substitutions, 1 to 7 natural amino acid substitutions, 1 to 8 natural amino acid substitutions, 2 to 3 natural amino acid substitutions, 2 to 4 natural amino acid substitutions, 2 to 5 natural amino acid substitutions, 2 to 6 natural amino acid substitutions, 2 to 7 natural amino acid substitutions, 2 to 8 natural amino acid substitutions, 2 to 9 natural amino acid substitutions, 3 or 4 natural amino acid substitutions, 3 to 5 natural amino acid substitutions, 3 to 6 natural amino acid substitutions, 3 to 7 natural amino acid substitutions, 3 to 9 natural amino acid substitutions, 4 or 5 natural amino acid substitutions, 4 to 6 natural amino acid substitutions, 4 to 7 amino acid substitutions, 4 to 9 natural amino acid substitutions, 5 or 6 natural amino acid substitutions, 5 to 7 amino acid substitutions, 5 to 9 natural amino acid substitutions, 6 or 7 natural amino acid substitutions, 6 to 9 natural amino acid substitutions, or 7 to 9 natural amino acid substitutions. In some embodiments, the IL-2 polypeptide comprises 3 natural amino acid substitutions, 4 natural amino acid substitutions, 5 amino acid substitutions, 6 natural amino acid substitutions, 7 natural amino acid substitutions, or 9 natural amino acid substitutions. In some embodiments, the IL-2 polypeptide comprises at most 4 natural amino acid substitutions, 5 natural amino acid substitutions, 6 natural amino acid substitutions, 7 natural amino acid substitutions, or 9 natural amino acid substitutions. In some embodiments, the IL-2 polypeptide further comprises up to 10 non-canonical amino acid substitutions. In some embodiments, the IL-2 polypeptide comprises 1, 2, 3, 4, 5, 6, 7, 8, 9 or 10 unnatural amino acid substitutions. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises unnatural amino acid substitutions at residues 41, 71, and 104. In some embodiments, the IL-2 polypeptide further comprises homoserine (Hse) 41, Hse 71, and Hse 104.

In some embodiments, the IL-2 polypeptide comprises at least one substitution or modification (e.g., attachment of a polymer) to the amino acid sequence of SEQ ID NO: 701. In some embodiments, the at least one substitution or modification has an impact on the ability of the IL-2 polypeptide to bind to one or more IL-2 receptor subunits. In some embodiments, the at least one substitution or modification diminishes the ability of the IL-2 polypeptide to bind to the IL-2 receptor a subunit. Further non-limiting examples such modifications are described in, for example, PCT Publication Nos. WO2021140416A2, WO2012065086A1, WO2019028419A1, WO2012107417A1, WO2018119114A1, WO2012062228A2, WO2019104092A1, WO2012088446A1, and WO2015164815A1, each of which is hereby incorporated by reference as if set forth herein in its entirety. In addition to modifications of IL-2 which may affect binding to one or more IL-2 receptor subunits (such as the alpha subunit), the IL-2 polypeptide provided herein may also comprises one or more modifications which improve the stability or pharmacokinetic properties of the IL-2 polypeptide. For example, the IL-2 polypeptide provided herein can comprise the modifications relative to SEQ NO: 701 which are contained in aldesluekin (Proleukin®) (SEQ ID NO: 702), namely a deletion of the N-terminal A residue of WT IL-2 and a C125S substitution relative to WT IL-2.

Non-limiting examples of modifications to IL-2 polypeptides include amino acid substitutions shown in Table 4 or 5 below. In some embodiments, the IL-2 polypeptide comprises 1, 2, 3, 4, 5, or more of the amino acid substitutions set forth in the Tables 4 and 5 below.

TABLE 4
IL-2 Substitution

| WT IL-2 | | |
|---|---|---|
| Residue | WT IL-2 | |
| Number* | Residue | Mutations |
| 35 | K | D, I, L, M, N, P, Q, T, Y |
| 36 | L | A, D, E, F, G, H, I, K, M, N, P, R, S, |
| | | W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, S, T, |
| | | V, Y |
| 43 | K | H, Y |
| 44 | F | K, Y |
| 45 | Y | A, D, E, G, K, L, N, Q, R, S, T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, R, S, T, |
| | | V, W, Y |
| 66 | L | A, F, Y |
| 67 | E | A, Y |
| 68 | E | V, Y |
| 72 | L | A, D, E, G, K, N, Q, R, S, T, Y |
| 125 | C | S |

TABLE 5
Additional IL-2 Substitutions

| WT IL-2 | | |
|---|---|---|
| Residue | WT IL-2 | |
| Number* | Residue | Mutations |
| 20 | D | T, Y |
| 35 | K | D, I, L, M, N, P, Q, T Y |
| 38 | R | A, D, G, K, N, P, S, Y |
| 42 | F | A, D, E, G, I, K, L, N, Q, R, S, T, V, |
| | | Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, |
| | | V, W, Y |
| 68 | E | V, Y |
| 72 | L | A, D, E, G, K, N, Q, R, S, T, Y |
| 125 | C | S |

In some embodiments, the IL-2 polypeptide comprises at least one modification is in the range of amino acid residues 30-75. In some embodiments, the IL-2 polypeptide comprises at least one polymer attachment to the residue at position 42 and/or 45 and/or an amino acid substitution at residue position 42 and/or 45. In some embodiments, one modification is at amino acid residue 42. In some embodiments, one modification is a F42Y substitution. In some embodiments, one modification is a polymer attached to residue F42Y. In some embodiments, one modification is at residue 45. In some embodiments, the modification at residue 45 is a polymer attached to residue 45. In some embodiments, the modification at residue 45 is a polymer attached to residue Y45. In some embodiments, the IL-2 polypeptide comprises a first polymer attached at residue F42Y and a second polymer attached at residue Y45. In some embodiments, the IL-2 polypeptide comprises a deletion of residue 1 from SEQ ID NO: 701. In some embodiments, the IL-2 polypeptide comprises a C125S substitution. In some embodiments, the IL-2 polypeptide further comprises one or more substitutions of a synthetic IL-2 polypeptide as provided herein (e.g., Hse or Nle substitutions).

In one embodiment an IL-2 polypeptide of an immunocytokine composition as provided herein (e.g., with a linker attached to a residue as provided herein, such as the N-terminal residue), further comprising a first polymer covalently attached at residue 42 and a second polymer covalently attached at residue 45, wherein residue position numbering of the IL-2 polypeptide is based on SEQ ID NO: 701 as a reference sequence. In some embodiments, the first polymer and the second polymer are the same. In some embodiments, the first polymer and the second polymer are different. In some embodiments, each polymer is attached through a tyrosine residue. In some embodiments, the IL-2 polypeptide comprises an amino acid sequence at least 80%, at least 85%, at least 90%, at least 95%, or at least 98% identical to the amino acid sequence of SEQ ID NO: 701. In some embodiments, the IL-2 polypeptide comprises an amino acid sequence at least 80%, at least 85%, at least 90%, at least 95%, or at least 98% identical to the amino acid sequence of SEQ ID NO: 702. In some embodiments, the IL-2 polypeptide comprises a C125S or C125A substitution. In some embodiments, the IL-2 polypeptide comprises a deletion of A1 from the sequence of SEQ ID NO: 701. In some embodiments, the IL-2 polypeptide comprises amino acid substitutions at 1, 2, 3, or 4 methionine residues from SEQ ID NO: 701. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises homoserine Hse 41, Hse 71, and Hse 104.

In some embodiments, the polypeptide sequence is at least about 80% identical to SEQ ID NO: 703. In some embodiments, the polypeptide sequence is at least about 90% identical to SEQ ID NO: 703. In some embodiments, the polypeptide sequence is at least about 95% identical to SEQ ID NO: 703. In some embodiments, the IL-2 polypeptide is synthetic.

In some embodiments, the IL-2 receptor beta/gamma biased IL-2 polypeptide a polymer attached to a residue of the IL-2 polypeptide (e.g., a polymer in addition to the linker attached at the point of attachment). In some embodiments, the polymer is attached to a different residue than the residue to which the linker is attached.

In some embodiments, the polymer is attached to an amino acid residue of the IL-2 polypeptide. In some embodiments, the polymer is attached to any amino acid residue of the IL-2 polypeptide (e.g., at a position corresponding to any one of positions 1-133 of SEQ ID NO: 701). In some embodiments, the polymer is attached at a non-terminal residue (e.g., a residue other than the C-terminal residue or N-terminal residue) of the IL-2 polypeptide (e.g., a residue at position corresponding to any one of positions 2-132 of SEQ ID NO: 701). In some embodiments, the polymer is attached at a terminal residue of the IL-2 polypeptide, wherein the IL-2 polypeptide has been extended or truncated by one or more amino acids relative to SEQ ID NO: 701 (e.g., the linker is attached to a residue corresponding to residue 2 of SEQ ID NO: 701 and residue 1 of SEQ ID NO: 701 has been deleted). In some embodiments, the polymer is attached to the N-terminal residue of the IL-2 polypeptide. In some embodiments, the polymer is attached to the N-terminal amine of the IL-2 polypeptide. In some embodiments, the polymer is attached to the C-terminal residue of the IL-2 polypeptide. In some embodiments, the polymer is attached to the C-terminal carboxyl group of the IL-2 polypeptide.

In some embodiments, the polymer is attached to the IL-2 polypeptide at a residue in a region comprising residues 2-132, wherein residue position numbering is based on SEQ ID NO: 701 as a reference sequence. In some embodiments, the polymer is attached to the IL-2 polypeptide at a residue in a region comprising residues 30-75. In some embodiments, the polymer is attached to the IL-2 polypeptide at a residue in a region comprising residues 35-55, residues 35-50, residues 35-45, residues 30-50, residues 40-45, residues 60-75, residues 60-70, residues 65-70, or residues 2-5. In some embodiments, the polymer is attached to the IL-2 polypeptide at a residue selected from residue 65, 66, 67, 68, 69, and 70. In some embodiments, the polymer is attached to the IL-2 polypeptide at a residue selected from residue 40, 41, 42, 43, 44, and 45. In some embodiments, the polymer is attached to the IL-2 polypeptide at residue 42 or 45. In some embodiments, the polymer is attached to the IL-2 polypeptide at residue 42. In some embodiments, the polymer is attached to the IL-2 polypeptide at residue 45.

In some embodiments, the polymer is attached to the IL-2 polypeptide at a residue which disrupts binding of the IL-2 polypeptide with the IL-2 receptor alpha subunit (IL-2R). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety. In some embodiments, the polymer is covalently attached at a residue selected from residues corresponding to residues 3, 34, 35, 36, 37, 38, 40, 41, 42, 43, 44, 45, 60, 61, 62, 63, 64, 65, 67, 68, 69, 71, 72, 103, 104, 105, and 107 of SEQ ID NO: 701. In some embodiments, the polymer is covalently attached at residue 1, 35, 37, 38, 41, 42, 43, 44, 45, 60, 61, 62, 64, 65, 68, 69, 71, 72, 104, 105, or 107 of the IL-2 polypeptide. In some embodiments, the polymer is covalently attached at residue 5. In some embodiments, the polymer is covalently attached at residue 38. In some embodiments, the polymer is covalently attached at residue 42. In some embodiments, the polymer is covalently attached at residue 45. In some embodiments, the polymer is covalently attached at residue 61. In some embodiments, the polymer is covalently attached at residue 65. In some embodiments, the polymer is covalently attached at residue 68.

In some embodiments, the residue to which the polymer is attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is a tyrosine. In some embodiments, the residue to which the polymer is attached is the natural amino acid in that position in SEQ ID NO: 701 (e.g., Y45 or A1).

In some embodiments, the polymer is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the 1 polymer is covalently attached to site-specifically to a natural amino acid.

In some embodiments, the polymer is attached to a tyrosine residue. In some embodiments, the polymer attached to the tyrosine residue has a structure:

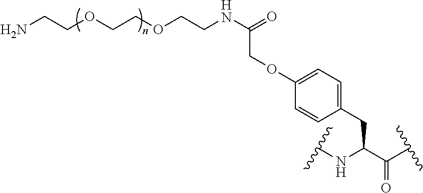

wherein n is an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, the polymer attached to the tyrosine residue is at residue F42Y. In some embodiments, the polymer attached to the tyrosine residue is at Y45. In some embodiments, the IL-2 polypeptide comprises two polymers attached to tyrosine residues at F42Y and Y45. In some embodiments, the two polymers are the same size.

In some embodiments, the polymer is attached at an unnatural amino acid residue. In some embodiments, the unnatural amino acid residue comprises a conjugation handle. In some embodiments, the conjugation handle facilitates the addition of the polymer to the IL-2 polypeptide. The conjugation handle can be any of the conjugation handles provided herein, and is preferably a different conjugation handle which is non-reactive with a conjugation handle used to attach or form part of the linker (where a conjugation handle is used to form the linker). In some embodiments, the polymer is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety. In some embodiments, the polymer is attached to an unnatural amino acid residue without use of a conjugation handle.

In some embodiments, the polymer is covalently attached at residue 42. In some embodiments, the polymer is covalently attached at residue F42E, F42D, F42Q, F42K, F42N, or F42Y. In some embodiments, the polymer is covalently attached at residue F42Y. In some embodiments, the polymer is covalently attached to an unnatural amino acid at residue 42.

In some embodiments, the polymer is covalently attached at residue 45. In some embodiments, the polymer is covalently attached at residue Y45, Y45E, Y45C, Y45D, Y45Q, Y45K, or Y45N. In some embodiments, the polymer is covalently attached at residue Y45. In some embodiments, the polymer is covalently attached to an unnatural amino acid at residue 45.

In some embodiments, the polymer is covalently attached at residue 65. In some embodiments, the polymer is covalently attached at residue P65C, P65D, P65Q, P65E, P65N, P65K, or P65Y. In some embodiments, the polymer is covalently attached to an unnatural amino acid at residue 65.

In some embodiments, the polymer is covalently attached at residue 5. In some embodiments, the polymer is covalently attached at residue S5C, S5D, S5Q, S5K, S5N, S5K, or S5Y. In some embodiments, the polymer is covalently attached to an unnatural amino acid at residue 5.

In some embodiments, the polymer is covalently attached at residue 1. In some embodiments, the polymer is covalently attached at residue A1. In some embodiments, the polymer is covalently attached to the N-terminal amine of the IL-2 polypeptide.

In some embodiments, the polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide) (PEG).

In some embodiments, the polymer has a molecular weight of from about 0.1 kDa to about 50 kDa. In some embodiments, the polymer has a molecular weight of from about 0.1 kDa to about 0.5 kDa from about 0.1 kDa to about 1 kDa, from about 0.1 kDa to about 2 kDa, from about 0.1 kDa to about 5 kDa from about 0.2 kDa to about 1 kDa, from about 0.2 kDa to about 2 kDa, from about 0.2 kDa to about 5 kDa, from about 0.2 kDa to about 10 kDa, from about 0.2 kDa to about 30 kDa, from about 0.5 kDa to about 2 kDa, from about 0.5 kDa to about 5 kDa, from about 0.5 kDa to about 10 kDa, from about 0.5 kDa to about 30 kDa, from about 1 kDa to about 5 kDa, from about 1 kDa to about 10 kDa, from about 1 kDa to about 30 kDa, from about 1 kDa to about 50 kDa, from about 2 kDa to about 10 kDa, from about 2 kDa to about 30 kDa, from about 2 kDa to about 50 kDa, from about 5 kDa to about 30 kDa, or from about 5 kDa to about 50 kDa. In some embodiments, the polymer has a molecular weight of at least about 0.2 kDa, at least about 0.5 kDa, at least about 1 kDa, at least about 2 kDa, at least about 5 kDa, at least about 10 kDa, or at least about 30 kDa. In some embodiments, the polymer has a molecular weight of at most about 30 kDa, at most about 10 kDa, at most about 5 kDa, at most about 2 kDa, at most about 1 kDa, at most about 0.5 kDa or at most about 0.2 kDa. In some embodiments, the polymer has a molecular weight of about 0.5 kDa, about 1 kDa, about 2 kDa, about 3 kDa, about 4 kDa, about 5 kDa, about 7.5 kDa, about 10 kDa, about 12.5 kDa, about 15 kDa, about 20 kDa, about 25 kDa, about 30 kDa, about 35 KDa, about 40 kDa, about 45 kDa, or about 50 kDa. In some embodiments, the polymer is a PEG polymer.

In some embodiments, the polymer is linear. In some embodiments, the polymer is a linear PEG polymer. In some embodiments, the polymer is branched. In some embodiments, the polymer is a branched PEG polymer. In some embodiments, the branched PEG polymer comprises a plurality of PEG chains from a central source molecule (e.g., a lysine or poly-lysine source molecule). In some embodiments, the polymer comprises from 1 to 10 polyethylene glycol chains. In some embodiments, the polymer comprises 1 polyethylene glycol chains to 10 polyethylene glycol chains. In some embodiments, the polymer comprises 1 polyethylene glycol chains to 2 polyethylene glycol chains, 1 polyethylene glycol chains to 4 polyethylene glycol chains, 1 polyethylene glycol chains to 6 polyethylene glycol chains, 1 polyethylene glycol chains to 10 polyethylene glycol chains, 2 polyethylene glycol chains to 4 polyethylene glycol chains, 2 polyethylene glycol chains to 6 polyethylene glycol chains, 2 polyethylene glycol chains to 10 polyethylene glycol chains, 4 polyethylene glycol chains to 6 polyethylene glycol chains, 4 polyethylene glycol chains to 10 polyethylene glycol chains, or 6 polyethylene glycol chains to 10 polyethylene glycol chains. In some embodiments, the polymer comprises 1 polyethylene glycol chains, 2 polyethylene glycol chains, 4 polyethylene glycol chains, 6 polyethylene glycol chains, or 10 polyethylene glycol chains. In some embodiments, the first water-soluble polymer comprises at least 1 polyethylene glycol chains, 2 polyethylene glycol chains, 4 polyethylene glycol chains, or 6 polyethylene glycol chains. In some embodiments, the first water-soluble polymer comprises at most 2 polyethylene glycol chains, 4 polyethylene glycol chains, 6 polyethylene glycol chains, or 10 polyethylene glycol chains. In some embodiments, the polymer comprises 4 polyethylene glycol chains.

In some embodiments, the polymer is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl), carboxylate, and ester (e.g., methyl ester, ethyl ester, etc.). In some embodiments, the polymer as an amine end-capped PEG.

In some embodiments, the IL-2 polypeptide comprises two polymers covalently attached to two separate residues of the IL-2 polypeptide. In some embodiments, the two polymers are a first polymer and a second polymer. Each of the first polymer and the second polymer can be attached to the IL-2 polypeptide at any of the residues as provided herein and can be any of the polymers provided herein (e.g., having any combination of sizes as provided herein). In some embodiments, both of the first polymer and the second polymer are the same size or about the same size. In some embodiments, both polymers are at most about 1 kDa. In some embodiments, one polymer is substantially larger than the other. In some embodiments, one polymer is at most about 1 kDa and the other polymer is at least about 5 kDa.

A non-limiting set of IL-2 polypeptides provided herein with various linker points of attachment and polymers as provided herein is shown in Table 6 below.

TABLE 6
Exemplary Polymer Attachment Sites to IL-2

| | Linker | Polymer 1 | Polymer 2 |
|---|---|---|---|
| IL-2 | Point of | Point of | Point of |
| Construct | Attachment | Attachment | Attachment |
| IL-2-A | N-terminus | Residue 42 | Residue 45 |
| IL-2-B | N-terminus | Residue 42 | None |
| IL-2-C | N-terminus | Residue 45 | None |
| IL-2-D | Residue 42 | Residue 45 | None |
| IL-2-E | Residue 42 | N-terminus | Residue 45 |
| IL-2-F | Residue 42 | N-terminus | None |
| IL-2-G | Residue 45 | Residue 42 | None |
| IL-2-H | Residue 45 | N-terminus | Residue 42 |
| IL-2-I | Residue 45 | N-terminus | None |
| IL-2-J | N-terminus | Residue 65 | None |
| IL-2-K | Residue 65 | N-terminus | None |

*Residue position numbering based on SEQ ID NO: 701 as a reference sequence

In some embodiments, the IL-2 polypeptide comprises the linker covalently attached to the N-terminus, a first polymer covalently attached at residue 42, and a second polymer covalently attached at residue 45. In some embodiments, the first polymer is covalently attached at residue F42Y. In some embodiments, the second polymer is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently to residue 42, a first polymer covalently attached at residue 45, and a second polymer covalently attached at the N-terminus. In some embodiments, the linker is attached at residue F42Y. In some embodiments, the first polymer is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently attached to residue 45, a first polymer covalently attached at residue 42, and a second polymer covalently attached at the N-terminus. In some embodiments, the first polymer is covalently attached at residue F42Y. In some embodiments, the linker is covalently attached at residue Y45. In some embodiments, the first polymer and the second polymer are different sizes. In some embodiments, the first polymer has a molecular weight of at most about 1 kDa and the second polymer has a molecular weight of at least about 5 kDa. In some embodiments, the first polymer has a molecular weight of from about 0.1 kDa to about 1 kDa and the second polymer has a molecular weight of from about 5 kDa to about 50 kDa. In some embodiments, the first polymer has a molecular weight of at least about 5 kDa and the second polymer has a molecular weight of at most about 1 kDa. In some embodiments, the first polymer has a molecular weight of from about 5 kDa to about 50 kDa and the second polymer has a molecular weight of from about 0.1 kDa to about 1 kDa. In some embodiments, the first polymer and the second polymer are the same or about the same size. In some embodiments, the first polymer and the second polymer each have a molecular weight of from about 0.1 kDa to about 1 kDa, about 0.2 kDa to about 1 kDa, or from about 0.5 kDa to about 1 kDa.

In some embodiments, the IL-2 polypeptide comprises the linker covalently attached to residue 45 and a polymer covalently attached at residue 42. In some embodiments, the linker is attached at residue Y45. In some embodiments, the polymer is attached at residue F42Y. In some embodiments, the polymer has a molecular weight of at most about 1 kDa. In some embodiments, the polymer has a molecular weight of from about 0.1 kDa to about 1 kDa. In some embodiments, the polymer has a molecular weight of at least about 5 kDa. In some embodiments, the polymer has a molecular weight of from about 5 kDa to about 50 kDa.

In some embodiments, the IL-2 polypeptide comprises the linker covalently attached to residue 42 and a polymer covalently attached at residue 45. In some embodiments, the linker is attached at residue F42Y. In some embodiments, the polymer is attached at residue Y45. In some embodiments, the polymer has a molecular weight of at most about 1 kDa. In some embodiments, the polymer has a molecular weight of from about 0.1 kDa to about 1 kDa. In some embodiments, the polymer has a molecular weight of at least about 5 kDa. In some embodiments, the polymer has a molecular weight of from about 5 kDa to about 50 kDa.

Exemplary IL-2 polypeptides to which polymers can be attached to provide IL-2 polypeptides with reduced alpha subunit binding and retention of beta/gamma subunit binding are shown in Table 4 below, as well as the sequence of WT IL-2 (SEQ ID NO: 701 of Table 7) and aldesleukin (SEQ ID NO: 702 of Table 7). In some embodiments, the IL-2 polypeptide of SEQ ID NO: 703 modified with polymers attached at residues F42Y and Y45 is used in an immunocytokine composition of the instant disclosure.

TABLE 7
Exemplary IL-2 Sequences

| SEQ ID | |
|---|---|
| NO | Sequence |
| 701 | APTSSSTKKTQLQLEHLLLDLQMILNGINNYKNPKLTRMLTFKFYMPKK |
| (WT IL-2) | ATELKHLQCLEEELKPLEEVLNLAQSKNFHLRPRDLISNINVIVLELKGSE |
| | TTFMCEYADETATIVEFLNRWITFCQSIISTLT |
| 702 | PTSSSTKKTQLQLEHLLLDLQMILNGINNYKNPKLTRMLTFKFYMPKKA |
| (aldesleukin) | TELKHLQCLEEELKPLEEVLNLAQSKNFHLRPRDLISNINVIVLELKGSET |
| | TFMCEYADETATIVEFLNRWITFSQSIISTLT |
| 703 | APTSSSTKKTQLQLEHLLLDLQ(Nle)ILNGINNYKNPKLTR(Ne)L(Hse)YK |
| | FY(Nle)PKKATELKHLQCLEEELKPLEEVL(Hse)LAQSKNFHLRPRDLISNI |
| | NVIVLELKGSETTF(Hse)CEYADETATIVEFLNRWITFSQSIISTLT |

In the table above, Ne is a norleucine residue and Hse is a homoserine residue.

One exemplary IL-2 of the instant disclosure which can be used to form a conjugated immunocytokine described herein is referred to herein as “2P72.” 2P72, its manufacture, properties, and derivatives thereof are described in detail in U.S. Pat. No. 11,633,488 (described therein as “Composition A2”). 2P72 has a base sequence as set forth in SEQ ID NO: 703 and contains polymers attached at residues F42Y and Y45 as depicted in the structures below:

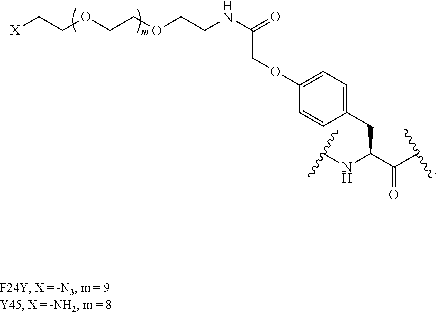

IL-2 Polypeptides Which Selectively Bind to IL-2 Receptor Alpha Subunit with Enhanced Binding to IL-2 Receptor Alpha Subunit

In some embodiments, an IL-2 polypeptide incorporated into an immunocytokine composition described herein is an IL-2 polypeptide which exhibits enhanced alpha subunit binding but lacks or has severely impaired binding to the IL-2 receptor beta and/or gamma subunits. Non-limiting examples of such IL-2 polypeptides can be found in US20230303649A1, the contents of which are incorporated herein by reference.

In some embodiments, a preferred IL-2 polypeptide having such characteristics is the IL-2 polypeptide of SEQ ID NO: 776 (e.g., an IL-2 polypeptide having Y31H, K35R, Q74P, N88D, C125S, Hse41, Hse71, Hse104, Nle23, Nle39, and Nle46 substitutions relative to WT IL-2). In some embodiments, the IL-2 polypeptide is the IL-2 polypeptide described as “Composition A” in US20230303649A1 (e.g., an IL-2 polypeptide having SEQ ID NO: 776 and an azide conjugation handle attached to the N-terminus as shown therein). Additional IL-2 polypeptides having similar properties are which are compatible with the instant disclosure are described in more detail below.

Such IL-2 polypeptides may display binding characteristics for the IL-2 receptor (IL-2R) that differ from wild-type IL-2 (SEQ ID NO:701) or aldesleukin (SEQ ID NO: 702). In one aspect, IL-2 polypeptides described herein have increased affinity for the IL-2R α complex. In some embodiments, the IL-2 polypeptides have an unmodulated affinity for the IL-2R βγ complex. In some embodiments, the IL-2 polypeptides have a reduced affinity for the IL-2R βγ complex. In some embodiments, the IL-2 polypeptides provided herein may comprise amino acid substitutions that enhance the binding affinity for the IL-2Rα receptor subunit. In some embodiments, the IL-2 polypeptides provided herein comprise amino acid substitutions that lower the IL-2 polypeptides affinity for the IL-2Rβ receptor subunit. In some embodiments, the IL-2 polypeptides have a biological activity of inducing fewer T-effector (T_{eff}) cells when administered in vivo compared to a wild type IL-2 or aldesleukin. In some embodiments, the IL-2 polypeptides provided herein have comparable ability (e.g., have an EC₅₀ no more than 10× greater, no more than 100× greater) to induce regulatory T-cells (T_{reg}) when administered in vivo compared to a wild type IL-2 or aldesleukin.

In some embodiments, the IL-2 polypeptides described herein contain modified amino acid residues. Such modifications can take the form of amino acid substitutions of a wild type IL-2 polypeptide such as the amino acid sequence of SEQ ID NO: 701, addition or deletion of amino acids from the sequence of SEQ ID NO: 701, or the addition of moieties to amino acid residues. In some embodiments, the IL-2 polypeptide described herein contains a deletion of the first amino acid from the sequence of SEQ ID NO: 701. In some embodiments, the IL-2 polypeptide described herein comprises a C125S substitution, using the sequence of SEQ ID NO: 701 as a reference sequence. In some embodiments, the IL-2 polypeptide described herein comprises substitutions at one or more residues selected from Y31, K35, Q74, and/or N88, wherein residue position numbering of the IL-2 polypeptide is based on SEQ ID NO: 701 as a reference sequence. These substitutions may be in combination with the C125S substitution and/or an N-terminal deletion, such as a deletion of the first amino acids from the sequence of SEQ ID NO: 701. In some embodiments, the Y31 substitution is a Y31H substitution. In some embodiments, the K35 substitutions is a K35R substitution. In some embodiments, the Q74 substitution is a Q74P substitutions. In some embodiments, the N88 substitution is an N88D substitution. In some embodiments, the IL-2 polypeptide comprises a Y31H substitution, a K35R substitution, and a Q74P substitution. In some embodiments, the IL-2 polypeptide comprises a Y311H substitution, a K35R substitution, a Q74P substitution, and an N88D substitution. In some embodiments, the IL-2 polypeptide comprises a Y31H substitution, a K35S substitution, a Q74P substitution, and a C125S substitution. In some embodiments, the IL-2 polypeptide comprises a Y31H substitution, a K35S substitution, a Q74P substitution, a N88D substitution, and a C125S substitution.

In some embodiments, the IL-2 polypeptide is a synthetic polypeptide. In some embodiments, the IL-2 polypeptide is synthesized by α-ketoacid-hydroxylamine (KAHA) amide-forming ligation. In some embodiments, the IL-2 polypeptide comprises unnatural amino acids, such as homoserine, which are used during the KAHA ligation reaction to join multiple polypeptide fragments to synthesize the full-length IL-2 polypeptide. In some embodiments, these are the only unnatural amino acids in the IL-2 polypeptide. In some embodiments, the IL-2 polypeptide comprises norleucine (Nle) residue substitutions at one or more methionine residues present in wild type IL-2 or aldesleukin. In some embodiments, the IL-2 polypeptide comprises norleucine residues at positions 23, 39, and 46.

In some embodiments, the IL-2 polypeptide as described herein can comprise one or more non-canonical amino acids (also referred to herein as “unnatural amino acids”). “Non-canonical” amino acids can refer to amino acid residues in D- or L-form that are not among the 20 canonical amino acids generally incorporated into naturally occurring proteins. In some embodiments, one or more amino acids of the IL-2 polypeptides are substituted with one or more non-canonical amino acids. Non-canonical amino acids include, but are not limited to N-alpha-(9-Fluorenylmethyloxycarbonyl)-L-azidolysine (Fmoc-L-Lys(N₃)—OH), N-alpha-(9-Fluorenylmethyloxycarbonyl)-L-biphenylalanine (Fmoc-L-Bip-OH), and N-alpha-(9-Fluorenylmethyloxycarbonyl)-O-benzyl-L-tyrosine (Fmoc-L-Tyr(Bzl)-OH, or their unprotected analogs.

Additionally, polymers may be added to IL-2 polypeptides. In some embodiments, the polymers are added in order to increase the half-life of the polypeptides. Such half-life extending polymers can be added to the N-terminus of the IL-2 polypeptides. The half-life extending polymers may be ofany size, including up to about 6 kDa, up to about 30 kDa, or up to about 50 kDa. In some embodiments, the half-life extending polymers are PEG polymers.

In some embodiments, the IL-2 polypeptide comprises one or more amino acid substitutions or deletions selected from the Table 8 below, wherein residue numbering is based on SEQ ID NO: 701 as a reference sequence.

TABLE 8
Exemplary IL-2 Substitutions

| WT IL-2 | | |
|---|---|---|
| Residue | WT IL-2 | |
| Number* | Residue | Substitutions or modification |
| 1 | A | Deletion |
| 18 | L | R, K |
| 22 | Q | N, H, K, Y, I, E |
| 23 | M | L, R, S, T, V, A |
| 29 | N | S |
| 31 | Y | H |
| 35 | K | R, E, D, Q |
| 37 | T | A, R |
| 46 | M | A |
| 48 | K | E, C |
| 69 | V | A |
| 71 | N | R |
| 74 | Q | P |
| 81 | R | A, G, S, T |
| 85 | L | V |
| 86 | I | V |
| 88 | N | A, D, E, F, G, H, I, M, Q, R, S, T, V, W |
| 89 | I | V |
| 92 | I | K, R |
| 125 | C | S, E, K, H, W, I, V, A |
| 126 | Q | A, C, D, E, F, G, H, I, K, L, M, N, R, S, T, |
| | | Y |

In some embodiments, the IL-2 polypeptide comprises one or more amino acid substitutions selected from the Table 9 below, wherein residue numbering is based on SEQ ID NO: 701 as a reference sequence.

TABLE 9
Additional IL-2 Substitutions

| WT IL-2 | | |
|---|---|---|
| Residue | WT IL-2 | |
| Number* | Residue | Mutations |
| 18 | L | R |
| 22 | Q | E |
| 23 | M | A |
| 29 | N | S |
| 31 | Y | H |
| 35 | K | R |
| 37 | T | A |
| 39 | M | A |
| 42 | F | (4-NH₂)-Phe |
| 46 | M | A |
| 48 | K | E |
| 69 | V | A |
| 71 | N | R |
| 74 | Q | P |
| 80 | L | F |
| 81 | R | D |
| 85 | L | V |
| 86 | I | V |
| 88 | N | D, Dgp (gp = O-(2-aminoethyl)-O′-(2- |
| | | aminoethyl)octaethylene glycol) |
| 89 | I | V |
| 92 | I | F |
| 126 | Q | T |

The IL-2 polypeptides described herein may also be synthesized chemically rather than expressed as recombinant polypeptides. The IL-2 polypeptides can be made by synthesizing one or more fragments of the full-length IL-2 polypeptides, ligating the fragments together, and folding the ligated full-length polypeptide. In some embodiments, the IL-2 polypeptide comprises Y31H, K35R, Q74P, and C125S substitutions and optionally a PEG polymer covalently attached to the N-terminus of the IL-2 polypeptide. In some embodiments, the IL-2 polypeptide comprises Y31H, K35R, Q74P, N88D, and C125S substitutions and optionally a PEG polymer covalently attached to the N-terminus of the IL-2 polypeptide. In some embodiments, the PEG polymer attached to the N-terminus acts as a linker which forms the attachment to the rest of the immunocytokine composition.

In some embodiments, the IL-2 polypeptides enhance regulatory T-cell (T_{reg}) cell proliferation or activation when administered to a subject. In some embodiments, the IL-2 polypeptides enhance T_{reg} proliferation or activation while sparing T-effector cells (T_{eff}) and/or natural killer (NK) cells when administered to a subject. In some embodiments, the IL-2 polypeptides increase Treg cells without substantially increasing CD8+ T cells and NK cells when administered to a subject.

In some embodiments, an IL-2 polypeptide is biased in favor of activation of T_{reg} cells compared to T_{eff} cells. In some embodiments, the IL-2 polypeptide comprises at least one amino acid substitutions at residues selected from Y31, K35, Q74, and N88, wherein residue position numbering of the IL-2 polypeptide is based on SEQ ID NO: 701 as a reference sequence. In some embodiments, the IL-2 polypeptide comprises amino acid substitutions at each of residues Y31, K35, and Q74, wherein residue position numbering of the IL-2 polypeptide is based on SEQ ID NO: 701 as a reference sequence. In some embodiments, the IL-2 polypeptide comprises the amino acid substitutions of Y31H, K35R, and Q74P. In some embodiments, the IL-2 polypeptide comprises amino acid substitutions at each of residues Y31, K35, Q74, and N88, wherein residue position numbering of the IL-2 polypeptide is based on SEQ ID NO: 701 as a reference sequence. In some embodiments, the IL-2 polypeptide comprises the amino acid substitutions of Y31H, K35R, Q74P, and N88D. In some embodiments, the IL-2 polypeptide does not comprise any additional substitutions that have a substantial impact on the binding of the IL-2 polypeptide to the IL-2Rα receptor.

In some embodiments, the IL-2 polypeptide exhibits substantially lower ability to activate T_{eff} cells than an IL-2 polypeptide of SEQ ID NO: 701 and/or SEQ ID NO: 702. In some embodiments, the IL-2 polypeptide retains the ability to activate T_{reg} cells. In some embodiments, the IL-2 polypeptide exhibits an enhanced ability to activate T_{reg} cells compared to an IL-2 polypeptide of SEQ ID NO: 701 and/or SEQ ID NO: 702 In some embodiments, the IL-2 polypeptide exhibits at least about 4× lower dissociation constant (K_{d}) of IL-2Rα than an IL-2 polypeptide of SEQ ID NO: 701 and/or SEQ ID NO: 702. In some embodiments, the IL-2 polypeptide exhibits a 2-fold to 10-fold lower dissociation constant (K_{d}) of IL-2Rα than an IL-2 polypeptide of SEQ ID NO: 701 and/or SEQ ID NO: 702.

In some embodiments, an IL-2 polypeptide that exhibits a greater affinity for IL-2 receptor a subunit than an IL-2 polypeptide of SEQ ID NO: 701 and/or SEQ ID NO: 702. In some embodiments, the affinity to IL-2 receptor a subunit is measured by dissociation constant (K_{d}). As used herein, the phrase “the K_{d} of the IL-2 polypeptide/IL-2 receptor a subunit” means the dissociation constant of the binding interaction of the IL-2 polypeptide and CD25.

In some embodiments, the K_{d} of the IL-2 polypeptide/IL-2 receptor α subunit is less than 10 nM. In some embodiments the K_{d} of the IL-2 polypeptide/IL-2 receptor α subunit is less than 10 nM, less than 7.5 nM, less than 5 nM, less than 4 nM, or less than 3 nM. In some embodiments, the K_{d} of the IL-2 polypeptide/IL-2 receptor α subunit between about 1 nM and 0.1 nM. In some embodiments, the K_{d} of the IL-2 polypeptide/IL-2 receptor α subunit between about 10 nM and about 0.1 nM. In some embodiments, the K_{d} of the IL-2 polypeptide/IL-2 receptor α subunit between about 10 nM and about 1 nM. In some embodiments, the K_{d} of the IL-2 polypeptide/IL-2 receptor α subunit between about 7.5 nM and about 0.1 nM. In some embodiments, the K_{d} of the IL-2 polypeptide/IL-2 receptor α subunit between about 7.5 nM and about 1 nM. In some embodiments, the K_{d} of the IL-2 polypeptide/IL-2 receptor α subunit between about 5 nM and about 0.1 nM. In some embodiments, the K_{d} of the IL-2 polypeptide/IL-2 receptor α subunit between about 5 nM and about 1 nM. In some embodiments, the K_{d} is measured by surface plasmon resonance.

In some embodiments, the IL-2 polypeptide that exhibits at least about a 10%, 50%, 100%, 250%, or 500% greater affinity for IL-2 receptor α subunit than an IL-2 polypeptide of SEQ ID NO: 701 and/or SEQ ID NO: 702. In some embodiments, the IL-2 polypeptide exhibits at most about a 500%, 750%, or 1000% greater affinity for IL-2 receptor α subunit than an IL-2 polypeptide of SEQ ID NO: 701 and/or SEQ ID NO: 702.

In some embodiments, the IL-2 polypeptide exhibits about 1.5-fold to about 10-fold greater affinity for IL-2 receptor α subunit than an IL-2 polypeptide of SEQ ID NO: 701 and/or SEQ ID NO: 702.

In some embodiments, the IL-2 polypeptide exhibits substantially the same binding affinity for the IL-2Rα as compared to an IL-2 polypeptide of SEQ ID NO: 701 and/or SEQ ID NO: 702. In some embodiments, the IL-2 polypeptide exhibits a K_{d} with IL-2Rα that is within about 2-fold, about 4-fold, about 6-fold, about 8-fold, or about 10-fold of the K_{d} between an IL-2 polypeptide of SEQ ID NO: 701 and/or SEQ ID NO: 702 and IL-2Rα.

In some embodiments, the IL-2 polypeptide exhibits reduced affinity for the IL-2 receptor β subunit (IL-2Rβ) as compared to an IL-2 polypeptide of SEQ ID NO: 701 and/or SEQ ID NO: 702. In some embodiments, the IL-2 polypeptide exhibits at least about 10-fold, at least about 25-fold, at least about 50-fold, at least about 100-fold, or at least about 500-fold fold lower affinity for the IL-2Rβ. In some embodiments, the IL-2 polypeptide exhibits at least about 100-fold lower affinity for IL-2Rβ. In some embodiments, the IL-2 polypeptide exhibits substantially no affinity for IL-2Rβ. In some embodiments, the affinity is measured as the dissociation constant K_{d} (e.g., a lower affinity correlating with a higher dissociation constant).

In some embodiments, the IL-2 polypeptide exhibits a binding affinity for IL-2Rβ which is at least 500 nM, at least 1000 nM, at least 5000 nM, at least 10000 nM, at least 50000 nM, or at least 100000 nM. In some embodiments, the IL-2 polypeptide exhibits substantially no binding affinity for IL-2Rβ.

In some embodiments, the IL-2 polypeptide exhibits an affinity for IL-2Rα which is at least about 30-fold greater, at least about 50-fold grater, at least about 75-fold greater, at least about 100-fold greater, at least about 500-fold greater, or at least about 1000-fold greater than for IL-2Rβ. In some embodiments, the IL-2 polypeptide exhibits an affinity for IL-2Rα which is at least about 100-fold greater than for IL-2Rβ. In some embodiments, the IL-2 polypeptide exhibits an affinity for IL-2Rα which is at least about 1000-fold greater than for IL-2Rβ.

In some embodiments, an IL-2 polypeptide described herein is capable of expanding a regulatory T-cell (T_{reg}) cell population. In some embodiments, an IL-2 polypeptide described herein spares expansion of effector T-cells (T_{eff}).

In some embodiments, an IL-2 polypeptide has a half maximal effective concentration (EC₅₀) for activation of T_{reg} cells that at most moderately reduced compared to an IL-2 polypeptide of SEQ ID NO: 701 and/or SEQ ID NO: 702. In some embodiments, activation of T_{reg} cells is measured by assessing change in STAT5 phosphorylation in a population of T cells when in contact with the IL-2 polypeptide. In some embodiments, a T_{reg} cell is identified by being CD4⁺, CD25+ and FoxP3⁺. In some embodiments, a T_{reg} cell is identified by also showing elevated expression of CD25 (CD25^{Hi}). In some embodiments, the IL-2 polypeptide has an EC₅₀ for activation of T_{reg} cells of at most about 100 nM, at most about 75 nM, at most about 50 nM, at most about 40 nM, at most about 35 nM, at most about 30 nM, or at most about 25 nM. In some embodiments, the IL-2 polypeptide has an EC₅₀ for activation of T_{reg} cells of at most about 50 nM, at most about 40 nM, at most about 35 nM, at most about 30 nM, or at most about 25 nM, at most about 20 nM, at most about 15 nM, at most about 10 nM, or at most about 5 nM. In some embodiments, the IL-2 polypeptide has an EC₅₀ for activation of T_{reg} cells of at most about 100 nM. In some embodiments, the IL-2 polypeptide has an EC₅₀ for activation of T_{reg} cells of at most about 50 nM. In some embodiments, the IL-2 polypeptide has an EC₅₀ for activation of T_{reg} cells of at most about 25 nM. In some embodiments, the IL-2 polypeptide has an EC₅₀ for activation of T_{reg} cells of from about 0.1 nM to about 100 nM, from about 1 nM to about 100 nM, from about 0.1 nM to about 50 nM, from about 1 nM to about 50 nM, from about 0.1 nM to about 25 nM, from about 1 nM to about 25 nM, from about 0.1 nM to about 10 nM, or from about 1 nM to about 10 nM.

In some embodiments, the IL-2 polypeptide has an EC₅₀ for activation of T_{reg} cells that is at most 2-fold, at most 5-fold, at most 10-fold, at most 20-fold, at most 50-fold, at most 100-fold, at most 200-fold, at most 500-fold, or at most 1000-fold greater compared to an IL-2 polypeptide of SEQ ID NO: 701 and/or SEQ ID NO: 702. In some embodiments, the IL-2 polypeptide has an EC₅₀ for activation of T_{reg} cells that is at most 2-fold greater. In some embodiments, the IL-2 polypeptide has an EC₅₀ for activation of T_{reg} cells that is at most 5-fold greater. In some embodiments, the IL-2 polypeptide has an EC₅₀ for activation of T_{reg} cells that is at most 10-fold greater. In some embodiments, the IL-2 polypeptide has an EC₅₀ for activation of T_{reg} cells that is at most 50-fold greater. In some embodiments, the IL-2 polypeptide has an EC₅₀ for activation of T_{reg} cells that is at most 100-fold greater. In some embodiments, the IL-2 polypeptide has an EC₅₀ for activation of T_{reg} cells that is at most 200-fold greater. In some embodiments, the IL-2 polypeptide has an EC₅₀ for activation of T_{reg} cells that is at most 500-fold greater. In some embodiments, the IL-2 polypeptide has an EC₅₀ for activation of T_{reg} cells that is at most 1000-fold greater.

In some embodiments, an IL-2 polypeptide has a half maximal effective concentration (EC₅₀) for activation of T_{eff} cells that is substantially greater compared to an IL-2 polypeptide of SEQ ID NO: 701 and/or SEQ ID NO: 702. In some embodiments, the T_{eff} cell is 1, 2, or 3 of a CD8 T_{eff} cell (e.g., CD8⁺), a Naïve CD8 cell (e.g., CD8⁺, CD45RA⁺), or a CD4 Con cell (e.g., CD4⁺, FoxP3⁻), or any combination thereof. In some embodiments, activation of cells is measured by assessing change in STAT5 phosphorylation in a population of T cells when in contact with the IL-2 polypeptide. In some embodiments, the IL-2 polypeptide has an EC₅₀ for activation of T_{eff} cells of at least about 10 nM, at least about 50 nM, at least about 100 nM, at least about 500 nM, at least about 1000 nM, at least about 2000 nM, at least about 3000 nM, at least about 4000 nM, or at least about 5000 nM. In some embodiments, the IL-2 polypeptide has an EC₅₀ for activation of T_{eff} cells of at least about 100 nM. In some embodiments, the IL-2 polypeptide has an EC₅₀ for activation of T_{eff} cells of at least about 500 nM. In some embodiments, the IL-2 polypeptide has an EC₅₀ for activation of T_{eff} cells of at least about 1000 nM. In some embodiments, the IL-2 polypeptide has an EC₅₀ for activation of T_{eff} cells of at least about 5000 nM. In some embodiments, the IL-2 polypeptide has an EC₅₀ for activation of T_{eff} cells of at least 10-fold, at least 20-fold, at least 50-fold, at least 100-fold, at least 500-fold, or at least 1000-fold greater compared to an IL-2 polypeptide of SEQ ID NO: 701 and/or SEQ ID NO: 702. In some embodiments, the IL-2 polypeptide has an EC₅₀ for activation of T_{eff} cells of at least 10-fold greater. In some embodiments, the IL-2 polypeptide has an EC₅₀ for activation of T_{eff} cells of at least 50-fold greater. In some embodiments, the IL-2 polypeptide has an EC₅₀ for activation of T_{eff} cells of at least 100-fold greater. In some embodiments, the IL-2 polypeptide has an EC₅₀ for activation of T_{eff} cells of at least 500-fold greater. In some embodiments, the IL-2 polypeptide has an EC₅₀ for activation of T_{eff} cells of at least 1000-fold greater.

In some embodiments, the IL-2 polypeptide exhibits a substantially greater ability to activate T_{reg} cells compared to T_{eff} cells. In some embodiments, a ratio of EC50 for activation of a T_{eff} cell type over EC50 for activation of a T_{reg} cell type is at least 10, at least 20, at least 50, at least 100, at least 150, or at least 200. In some embodiments, a ratio of EC50 for activation of a T_{eff} cell type over EC50 for activation of a T_{reg} cell type is at least 100. In some embodiments, a ratio of EC50 for activation of a T_{eff} cell type over EC50 for activation of a T_{reg} cell type is at least 200. In some embodiments, a ratio of EC50 for activation of a T_{eff} cell type over EC50 for activation of a T_{reg} cell type is at least 300. In some embodiments, a ratio of EC50 for activation of a T_{eff} cell type over EC50 for activation of a T_{reg} cell type is at least 500. In some embodiments, a ratio of EC50 for activation of a T_{eff} cell type over EC50 for activation of a T_{reg} cell type is at least 1000.

In some embodiments, the level of activation is measured after about 0.5 h to about 1 h after incubation with the IL-2 polypeptide (e.g., 0.5 h to 1 h before fixing the cells for in in vitro experiment).

In some embodiments, the IL-2 polypeptide comprising one or more amino acid substitutions. In some embodiments, the amino acid substitutions affect the binding properties of the IL-2 polypeptide to IL-2 receptor subunits (e.g. alpha, beta, or gamma subunits) or to IL-2 receptor complexes (e.g. IL-2 receptor αβγ complex or βγ complex). In some embodiments, the amino acid substitutions are at positions on the interface of binding interactions between the IL-2 polypeptide and an IL-2 receptor subunit or an IL-2 receptor complex. In some embodiments, the amino acid substitutions cause an increase in affinity for the IL-2 receptor αβγ complex or alpha subunit. In some embodiments, the amino acid substitutions cause a decrease in affinity for the IL-2 receptor βγ complex or beta subunit.

In some embodiments, the IL-2 polypeptide comprises natural amino acid substitutions relative to WT IL-2 (SEQ ID NO: 701). In some embodiments, the IL-2 polypeptide comprises up to seven natural amino acid substitutions. In some embodiments, the IL-2 polypeptide comprises up to six amino acid substitutions. In some embodiments, the IL-2 polypeptide comprises up to five amino acid substitutions. In some embodiments, the IL-2 polypeptide comprises up to four amino acid substitutions. In some embodiments, the IL-2 polypeptide comprises up to three amino acid substitutions. In some embodiments, the IL-2 polypeptide comprises from three to seven, three to six, three to five, three to four, four to seven, four to six, four to five, five to seven, five to six, or six to seven natural amino acid substitutions. In some embodiments, the IL-2 polypeptide comprises at least one, at least two, at least three, at least four, at least five, or at least six amino acid substitutions.

In some embodiments, an IL-2 polypeptide provided herein comprises natural amino acid substitutions at at least one of Y31, K35, Q74, and N88D wherein residue position numbering of the IL-2 polypeptide is based on SEQ ID NO: 701 as a reference sequence. In some embodiments, the IL-2 polypeptide comprises natural amino acid substitutions at at least two of Y31, K35, Q74, and N88. In some embodiments, the IL-2 polypeptide comprises natural amino acid substitutions at at least three of Y31, K35, Q74, and N88. In some embodiments, the IL-2 polypeptide. In some embodiments, the IL-2 polypeptide comprises natural amino acid substitutions at each of Y31, K35, Q74, and N88. In some embodiments, the IL-2 polypeptide comprises the amino acid substitutions Y31H, K35R, Q74P, and N88D. In some embodiments, the IL-2 polypeptide further comprises an optional C125 substitution (e.g., C125S or C125A). In some embodiments, the IL-2 polypeptide further comprises an optional A1 deletion or substitution of residue A1. In some embodiments, the IL-2 polypeptide further comprises an optional A1 deletion.

In some embodiments, an IL-2 polypeptide provided herein comprises a Y31 substitution wherein residue position numbering of the IL-2 polypeptide is based on SEQ ID NO: 701 as a reference sequence. In some embodiments, the Y31 substitution is for an aromatic amino acid. In some embodiments, the Y31 substitution is for a basic amino acid. In some embodiments, the basic amino acid is weakly basic. In some embodiments, the Y31 substitution is selected from Y31F, Y31H, Y31W, Y31R, and Y31K. In some embodiments, the Y31 substitution is Y31H.

In some embodiments, an IL-2 polypeptide provided herein comprises a K35 substitution, wherein residue position numbering of the IL-2 polypeptide is based on SEQ ID NO: 701 as a reference sequence. In some embodiments, the K35 substitution is for a basic amino acid. In some embodiments, the K35 substitution is for a positively charged amino acid. In some embodiments, the K35 substitution is K35R, K35E, K35D, or K35Q. In some embodiments, the K35 substitution is K35R.

In some embodiments, an IL-2 polypeptide provided herein comprises a Q74 substitution, wherein residue position numbering of the IL-2 polypeptide is based on SEQ ID NO: 701 as a reference sequence. In some embodiments, the Q74 substitution is a cyclic amino acid. In some embodiments, the cyclic amino acid comprises a cyclic group covalently attached to the alpha carbon and the nitrogen attached to the alpha carbon. In some embodiments, the Q74 substitution is Q74P.

In some embodiments, an IL-2 polypeptide provided herein comprises a N88 substitution, wherein residue position numbering of the IL-2 polypeptide is based on SEQ ID NO: 701 as a reference sequence. In some embodiments, the N88 substitution is a charged amino acid residue. In some embodiments, the N88 substitution is a negatively charged amino acid residue. In some embodiments, the N88 substitution is N88D or N88E. In some embodiments, the N88 substitution is N88D or N88E. In some embodiments, the N88 substitution is N88D.

In some embodiments, an IL-2 polypeptide comprises a C125 substitution, wherein residue position numbering of the IL-2 polypeptide is based on SEQ ID NO: 701 as a reference sequence. In some embodiments, the C125 substitution stabilizes the IL-2 polypeptide. In some embodiments, the C125 substitution does not substantially alter the activity of the IL-2 polypeptide. In some embodiments, the IL-2 polypeptide comprises a C125S substitution. In some embodiments, the IL-2 polypeptide comprises a C125A substitution.

In some embodiment, an IL-2 polypeptide comprises a modification at residue A1, wherein residue position numbering of the IL-2 polypeptide is based on SEQ ID NO: 701 as a reference sequence. In some embodiments, the modification is an A1 deletion.

In some embodiments, the IL-2 polypeptide comprises additional amino acid substitutions. In some embodiments, the IL-2 polypeptide comprises an additional amino acid substitution that has an effect on binding to the IL-2 receptor alpha subunit or αβγ complex. In some embodiments, the IL-2 polypeptide comprises an additional amino acid substitution that has an effect on binding to the IL-2 receptor beta subunit or βγ complex. In some embodiments, the IL-2 polypeptide comprises at least one additional amino acid substitution selected from Table 8 or Table 9. In some embodiments, the IL-2 polypeptide comprises at least one amino acid substitution at residue E15, N29, N30, T37, K48, V69, N71, N88, 189, or 192. In some embodiments, the IL-2 polypeptide comprises at least one amino acid substitution at residue E15, N29, N30, T37, K48, V69, N71, 189, or 192. In some embodiments, the IL-2 polypeptide comprises 1, 2, 3, or 4 natural amino acid substitutions at residues selected from E15, N29, N30, T37, K48, V69, N71, N88, 189, or 192. n some embodiments, the IL-2 polypeptide comprises 1, 2, 3, or 4 natural amino acid substitutions at residues selected from E15, N29, N30, T37, K48, V69, N71, 189, or 192. In some embodiments, the IL-2 polypeptide comprises 1 natural amino acid substitutions at residues selected from E15, N29, N30, T37, K48, V69, N71, N88, 189, or 192. In some embodiments, the IL-2 polypeptide comprises 2 In some embodiments, the IL-2 polypeptide comprises up to 2 natural amino acid substitutions at residues selected from E15, N29, N30, T37, K48, V69, N71, N88, 189, or 192. In some embodiments, the IL-2 polypeptide comprises up to 3 natural amino acid substitutions at residues selected from E15, N29, N30, T37, K48, V69, N71, N88, 189, or 192. In some embodiments, the additional amino acid substitution comprises E15A, E15G, or E15S. In some embodiments, the additional amino acid substitution comprises N29S. In some embodiments, the additional amino acid substitution comprises N30S. In some embodiments, the additional amino acid substitution comprises T37A or T37R. In some embodiments, the additional amino acid substitution comprises K48E. In some embodiments, the additional amino acid substitution comprises V69A. In some embodiments, the additional amino acid substitution comprises N71R. In some embodiments, the additional amino acid substitution comprises N88A, N88D, N88E, N88F, N88G, N88H, N88I, N88M, N88Q, N88R, N88S, N88T, N88V, or N88W. In some embodiments, the additional amino acid substitution comprises N88D. In some embodiments, the additional amino acid substitution comprises I89V. In some embodiments, the additional amino acid substitution comprises 192K or 192R.

In some embodiments, an IL-2 polypeptide provided herein comprises substitutions at Y31, K35, Q74, and optionally C125S. In some embodiments, the IL-2 polypeptide does not comprise any additional substitutions which substantially affect binding to the IL-2 receptor alpha subunit or αβγ complex. In some embodiments, the IL-2 polypeptide does not comprise an additional amino acid substitution that has an effect on binding to the IL-2 receptor beta subunit or βγ complex. In some embodiments, the IL-2 polypeptide does not comprise any additional natural amino acid substitutions at residues E15, N29, N30, T37, K48, V69, N71, N88, 189, or 192. In some embodiments, the IL-2 polypeptide does not comprise any additional amino acid substitutions at residues E15, N29, N30, T37, K48, V69, N71, N88, 189, or 192. In some embodiments, the IL-2 polypeptide does not have a V69 substitution. In some embodiments, the IL-2 polypeptide does not have a V69A substitution. In some embodiments, the IL-2 polypeptide does not have a K48 substitution. In some embodiments, the IL-2 polypeptide does not have a K48E substitution. In some embodiments, the IL-2 polypeptide does not comprise a substitution at V69 or K48. In some embodiments, the IL-2 polypeptide does not comprise a substitution at either of V69 or K48. In some embodiments, the IL-2 polypeptide does not comprise a V69A or K48E substitution. In some embodiments, the IL-2 polypeptide does not comprise either a V69A or K48E substitution.

In some embodiments, an IL-2 polypeptide provided herein comprises substitutions at Y31, K35, Q74, N88, and optionally C125S. In some embodiments, the IL-2 polypeptide does not comprise any additional substitutions which substantially affect binding to the IL-2 receptor alpha subunit or αβγ complex. In some embodiments, the IL-2 polypeptide does not comprise an additional amino acid substitution that has an effect on binding to the IL-2 receptor beta subunit or βγ complex. In some embodiments, the IL-2 polypeptide does not comprise any additional natural amino acid substitutions selected from positions identified in Table 8 or Table 9. In some embodiments, the IL-2 polypeptide does not comprise any additional amino acid substitutions selected from Table 8 or Table 9. In some embodiments, the IL-2 polypeptide does not comprise any additional natural amino acid substitutions at residues E15, N29, N30, T37, K48, V69, N71, 189, or 192. In some embodiments, the IL-2 polypeptide does not comprise any additional amino acid substitutions at residues E15, N29, N30, T37, K48, V69, N71, 189, or 192. In some embodiments, the IL-2 polypeptide does not have a V69 substitution. In some embodiments, the IL-2 polypeptide does not have a V69A substitution. In some embodiments, the IL-2 polypeptide does not have a K48 substitution. In some embodiments, the IL-2 polypeptide does not have a K48E substitution. In some embodiments, the IL-2 polypeptide does not comprise a substitution at V69 or K48. In some embodiments, the IL-2 polypeptide does not comprise a substitution at either of V69 or K48. In some embodiments, the IL-2 polypeptide does not comprise a V69A or K48E substitution. In some embodiments, the IL-2 polypeptide does not comprise either a V69A or K48E substitution.

In some embodiments, an IL-2 polypeptide provided herein comprises an N-terminal deletion. In some embodiments, the N-terminal deletion is of 1, 2, 3, 4, 5, 6, 7, 8, 9, 10, 11, 12, 13, 14, 15, or more amino acids. In some embodiments, the N-terminal deletion is of at least 1 amino acid. In some embodiments, the N-terminal deletion is of at least 1, 2, 3, 4, 5, 6, 7, 8, 9, or 10 amino acids. In some embodiments, the N-terminal deletion is from 1 to 15 amino acids. In some embodiments, the N-terminal deletion is a deletion of a single amino acid (e.g., an A1 deletion of SEQ ID NO: 701). [0246]n some embodiments, the unnatural amino acid substitutions provided herein can be incorporated into an IL-2 polypeptide in addition to any combination of natural amino acid substitutions provided herein, unless otherwise specified. For example, where an IL-2 polypeptide comprises, for example, Y31H, K35R, and Q74P natural amino acid substitutions is described, it is expressly contemplated that the IL-2 polypeptide can also comprise unnatural amino acid substitutions (e.g., Hse41, Hse71, Hse104, Nle23, Nle39, and Nle46). As another example, where an IL-2 polypeptide provided herein is described as having Y31H, K35R, Q74P, and N88D natural amino acid substitutions, the IL-2 polypeptide can further comprise unnatural amino acid substitutions (e.g., Hse41, Hse71, Hse104, Nle23, Nle39, and Nle46). In particular, any combination of natural amino acid substitutions present in a recombinant IL-2 polypeptide provided herein can also be incorporated into a synthetic version of the IL-2 polypeptide (e.g., the corresponding IL-2 polypeptide containing, for example, Hse41, Hse71, Hse104, Nle23, Nle39, and Nle46).

In some embodiments, the IL-2 polypeptide comprises one or more unnatural amino acid substitutions. In some embodiments, the IL-2 polypeptide comprises at least two unnatural amino acid substitutions. In some embodiments, the IL-2 polypeptide comprises at least one amino acid substitution at a residue selected from Y31, K35, Q74, and N88, wherein residue position numbering of the IL-2 polypeptide is based on SEQ ID NO: 701 as a reference sequence. In some embodiments, the IL-2 polypeptide comprises a homoserine (Hse) residue located in any one of residues 36-45. In some embodiments, the IL-2 polypeptide comprises a Hse residue located in any one of residues 61-81. In some embodiments, the IL-2 polypeptide comprises a Hse residue located in any one of residues 94-114. In some embodiments, the IL-2 polypeptide comprises 1, 2, 3, or more Hse residues. In some embodiments, the IL-2 polypeptide comprises Hse41, Hse71, Hse104, or a combination thereof. In some embodiments, the IL-2 polypeptide comprises Hse41, Hse71, and Hse104. In some embodiments, the IL-2 polypeptide comprises at least two amino acid substitutions, wherein the at least two amino acid substitutions are selected from (a) a homoserine (Hse) residue located in any one of residues 36-45; (b) a homoserine residue located in any one of residues 61-81; and (c) a homoserine residue located in any one of residues 94-114. In some embodiments, the IL-2 polypeptide comprises Hse41 and Hse71. In some embodiments, the IL-2 polypeptide comprises Hse41 and Hse104. In some embodiments, the IL-2 polypeptide comprises Hse71 and Hse104. In some embodiments, the IL-2 polypeptide comprises Hse41. In some embodiments, the IL-2 polypeptide comprises Hse71. In some embodiments, the IL-2 polypeptide comprises Hse104. In some embodiments, the IL-2 polypeptide comprises 1, 2, 3, or more norleucine (Nle) residues. In some embodiments, the IL-2 polypeptide comprises a Nle residue located in any one of residues 18-28. In some embodiments, the IL-2 polypeptide comprises one or more Nle residues located in any one of residues 34-50. In some embodiments, the IL-2 polypeptide comprises a Nle residue located in any one of residues 20-60. In some embodiments, the IL-2 polypeptide comprises three Nle substitutions. In some embodiments, the IL-2 polypeptide comprises Nle23, Nle39, and Nle46. In some embodiments, the IL-2 polypeptide comprises SEQ ID NO: 775. In some embodiments, the IL-2 polypeptide comprises SEQ ID NO: 775 with an A1 deletion.

In some embodiments, the IL-2 polypeptide comprises an amino acid sequence of SEQ ID NO: 776. In some embodiments, the IL-2 polypeptide comprises an amino acid sequence at least 85% identical to the sequence of SEQ ID NO: 775. In some embodiments, the IL-2 polypeptide comprises an amino acid sequence at least 85%, at least 90%, at least 95%, or at least 98% identical to the sequence of SEQ ID NO: 775, wherein each residue which is substituted in SEQ ID NO: 775 relative to SEQ ID NO: 1 is retained.

In some embodiments, an IL-2 polypeptide described herein comprises at least about 80%, at least about 85%, at least about 90%, at least about 95%, at least about 96%, at least about 97%, at least about 98%, or at least about 99% sequence identity to SEQ ID NO: 775. In some embodiments, an IL-2 polypeptide described herein comprises at least about 80%, at least about 85%, at least about 90%, at least about 95%, at least about 96%, at least about 97%, at least about 98%, or at least about 99% sequence identity to SEQ ID NO: 775. In some embodiments, the sequence identity is measured by protein-protein BLAST algorithm using parameters of Matrix BLOSUM62, Gap Costs Existence:11, Extension:1, and Compositional Adjustments Conditional Compositional Score Matrix Adjustment.

In some embodiments, the IL-2 polypeptide comprises a modification of a terminal residue (e.g., the N-terminal residue or the C-terminal residue) which comprises a polymer. In some embodiments, the modification to the terminal residue comprises the attachment of a conjugation handle to the terminal residue of the IL-2 polypeptide. In some embodiments, the conjugation handle is attached to the IL-2 polypeptide through the N-terminal amino group or the C-terminal carboxyl group of the IL-2 polypeptide. In some embodiments, the conjugation handle is attached to the IL-2 polypeptide through the N-terminal amino group of the IL-2 polypeptide. In some embodiments, the conjugation handle is attached to the N-terminal amino group of the IL-2 polypeptide through a glutaryl-amino-PEG linker. In some embodiments, the conjugation handle is attached to the N-terminal amino group of the IL-2 polypeptide through an adduct having a structure

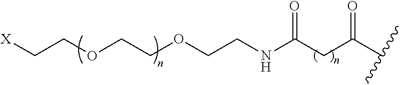

wherein each n is independently an integer from 1-30 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30), and wherein X is a conjugation handle (e.g., an azide or other conjugation handle provided herein, such as a DBCO group). In some embodiments, the IL-2 polypeptide will comprise the adduct above, but the conjugation handle X is replaced with a reaction product of the conjugation handle and a complementary conjugation handle (e.g., a 1,2,3 triazole) linking the IL-2 polypeptide to an additional moiety (e.g., a larger polymer or an additional polypeptide). In some embodiments, the N-terminal amino group of the IL-2 polypeptide comprises an adduct having a structure

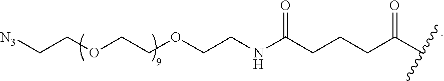

In some embodiments, a herein described IL-2 polypeptide comprises one or more polymers covalently attached thereon. In some embodiments, the described IL-2 polypeptide comprises 1, 2, 3, 4, 5, 6, 7, 8, 9, 10, or more polymers covalently attached to the IL-2 polypeptide. In some embodiments, the described IL-2 polypeptide comprises a polymer covalently attached to the N-terminus of the IL-2 polypeptide. The polymers provided herein may attached directly to a residue of the IL-2 polypeptide, may be attached through a small linking group (e.g., attached through a reaction with a conjugation handle incorporated into the IL-2 polypeptide).

The polymer as provided herein can be attached at any desired residue of the IL-2 polypeptide. In some embodiments, it is preferable that the polymer be attached at a residue which does not impact binding of the IL-2 polypeptide with the IL-2 receptor or a specific IL-2 receptor subunit (e.g., the IL-2 receptor alpha subunit). In some embodiments, the polymer is attached at or near the N-terminus of the IL-2 polypeptide. In some embodiments, the polymer is attached to the N-terminus of the IL-2 polypeptide. In some embodiments, the N-terminus is residue A1 of the IL-2 polypeptide, wherein residue position numbering is based on SEQ ID NO: 701 as a reference sequence. In some embodiments, the N-terminus is residue P2 of the IL-2 polypeptide, wherein residue position numbering is based on SEQ ID NO: 701 as a reference sequence (e.g., the IL-2 polypeptide comprises a deletion of residue A1 from the sequence). In some embodiments, the polymer is attached at a residue position which blocks or diminished binding of the IL-2 polypeptide with the IL-2 receptor beta subunit. Such residues positions are provided in U.S. Patent Publication Number 20200231644A1, which is hereby incorporated by reference as if set forth herein in its entirety, and include, for example, residue positions K8, K9, L12, E15, H16, L19, D20, Q22, M23, N26, D84, N88, E95, and Q126.

Non-limiting examples of IL-2 polypeptides which retain binding to the IL-2 receptor alpha but are detuned with respect to the beta and gamma subunits include those described in US20230303649A1. In some embodiments, an IL-2 polypeptide of an immunocytokine composition described herein is one described in US20230303649A1 (e.g., one described in Table 5 therein). In some embodiments, an IL-2 polypeptide of an immunocytokine composition described herein is one shown in Table 10 below.

TABLE 10
Exemplary IL-2 Polypeptides

| SEQ ID NO/ | | |
|---|---|---|
| Identifier | Substitutions | Sequence |
| 775 | M23Nle, Y31H, K35R, | APTSSSTKKT QLQLEHLLLD LQXILNGINN |
| (IL-2 | M39Nle, T41Hse, M46Nle | HKNPRLTRXL ZFKFYXPKKA TELKHLQCLE |
| variant 2P74) | N71Hse, Q74P, N88D, | EELKPLEEVL ZLAPSKNFHL RPRDLISDIN |
| | M104Hse, C125S | VIVLELKGSE TTFZCEYADE TATIVEFLNR |
| | X = Nle, Z = Hse | WITFSQSIIS TLT |

Modifications which Reduce Heparin Binding

In some embodiments, an IL-2 polypeptide described herein (e.g., any of the IL-2 polypeptides described herein) exhibits reduced binding to heparin. In some embodiments, reducing the binding of the IL-2 polypeptide to heparin results an IL-2 polypeptide (or an immunocytokine composition) which exhibits improved in vivo stability, biodistribution, and/or PK properties. In some embodiments, the IL-2 polypeptide comprises one or more modifications which reduce the binding of the IL-2 polypeptide to heparin. Any of the IL-2 polypeptides described herein can include such modifications (e.g., any of the IL-2 polypeptides described herein as having reduced binding to the IL-2 receptor alpha, any of the IL-2 polypeptides described herein as having reduced binding to the IL-2 receptor beta, or any of the IL-2 polypeptides described herein described as retaining binding to the IL-2 receptor alpha with reduced binding to the IL-2 receptors beta and/or gamma, or an activatable IL-2 polypeptide described herein).

In some embodiments, the IL-2 polypeptide of a multifunctional immunocytokine described herein comprises a modified B′C′ loop region of the IL-2 polypeptide. The B′C′ loop region refers to the amino acids which form the linkage between helixes B and C of IL-2 (e.g., human IL-2). The B′C′ loop region contains the amino acids positioned between amino acids 73 and 84 of wild type human IL-2 (SEQ ID NO: 701). In some embodiments, the modified B′C′ loop region of the IL-2 polypeptide comprises a deletion of one or more amino acids of the B′C′ loop region between amino acids 73 and 84 of the IL-2 polypeptide. In some embodiments, the modified B′C′ loop region of the IL-2 polypeptide an insertion of an exogenous peptide into the B′C′ loop region. In some embodiments, the modified B′C′ loop region of the IL-2 polypeptide comprises a deletion of one or more amino acids of the B′C′ loop region between amino acids 73 and 84 of the IL-2 polypeptide and an insertion of an exogenous peptide into the B′C′ loop region.

In some embodiments, the modified B′C′ loop region comprises a deletion of 1, 2, 3, 4, 5, 6, 7, 8, 9 or 10 amino acids between amino acids 73 and 84 of the IL-2 polypeptide. In some embodiments, the modified B′C′ loop region comprises a deletion of each amino acid 73 and 84 of the IL-2 polypeptide. In some embodiments, the modified B′C′ loop region comprises a deletion of each amino acid 73 and 84 of the IL-2 polypeptide and insertion of an exogenous peptide.

In some embodiments, the modified B′C′ loop region comprises insertion of an exogenous peptide. In some embodiments, the exogenous peptide comprises the sequence GDGSIN (SEQ ID NO: 700). In some embodiments, the exogenous peptide consists of the sequence GDGSIN (SEQ ID NO: 700). In some embodiments, the modified B′C′ loop region comprises a deletion of each amino acid between amino acids 73 and 84 of the IL-2 polypeptide and an insertion of an exogenous peptide having the sequence GDGSIN (SEQ ID NO: 700) (i.e., the amino acids between 73 and 84 of the IL-2 polypeptide are replaced with the sequence GDGSIN (SEQ ID NO: 700)). In some embodiments, the IL-2 polypeptide comprises a peptide sequence having at least 80%, 85%, 90%, 95%, 96%, 97%, 98%, 99%, or 100% sequence identity to amino acids 1-73 of SEQ ID NO: 701 and a peptide sequence having at least 80%, 85%, 90%, 95%, 96%, 97%, 98%, 99%, or 100% sequence identity to amino acids 84-133 of SEQ ID NO: 701. In some embodiments, the IL-2 polypeptide comprises a peptide sequence having at least 80%, 85%, 90%, 95%, 96%, 97%, 98%, 99%, or 100% identity to SEQ ID NO: 783.

In some embodiments, the IL-2 polypeptide of a multifunctional immunocytokine described herein comprises a substitution at one or more amino acids selected from residue 32, residue 35, residue 38, residue 76, residue 81, or residue 83. In some embodiments, the IL-2 polypeptide comprises a K32S, K35E, R38A, K76A, R81S, or R83S substitution. In some embodiments, the IL-2 polypeptide comprises K32S. In some embodiments, the IL-2 polypeptide comprises K35E. In some embodiments, the IL-2 polypeptide comprises R38A. In some embodiments, the IL-2 polypeptide comprises K76A. In some embodiments, the IL-2 polypeptide comprises R81S. In some embodiments, the IL-2 polypeptide comprises R83S. In some embodiments, the IL-2 polypeptide comprises K32S, K35E, and R38A substitutions. In some embodiments, the IL-2 polypeptide comprises K76A and R81S substitutions. In some embodiments, the IL-2 polypeptide comprises K76A, R81S, and R81S substitutions.

### Activatable IL-2 Polypeptides

In some embodiments, an IL-2 polypeptide described herein used in an immunocytokine composition (e.g., any of the IL-2 polypeptides described herein, such as the alpha-competent, beta-gamma detuned IL-2 polypeptides or the beta-gamma competent, alpha-detuned IL-2 polypeptides) is an activatable IL-2 polypeptide. In some embodiments, the activatable IL-2 polypeptide comprises a cleavable peptide, in particular a cleavable peptide, attached to a side chain of a residue of the IL-2 polypeptide which, upon cleavage, converts the activatable IL-2 polypeptide into an active form (e.g., the IL-2 related activity of the IL-2 polypeptide enhances following cleavage).

Examples of activatable IL-2 polypeptides and strategies for generating activatable IL-2 polypeptides compatible with the instant disclosure are described in WO2024150175A1 (corresponding US Patent Publication No. US20240417436A1), the contents of which are incorporated herein by reference as if set forth herein in its entirety. Further examples of such activatable IL-2 polypeptides incorporated into immunocytokines are described in WO2024150174A1 (corresponding US Patent Publication No. US20240417436A1), which is also incorporated herein by reference. Any of the strategies for generating an activatable IL-2 polypeptide described therein are contemplated as being applicable to any of the IL-2 polypeptides of the instant disclosure. In some embodiments, the activatable IL-2 polypeptide is one described in US Patent Publication No. US20240417436A1, in particularly in Table 3 therein (e.g., CMP-319 described therein).

Exemplary cleavable peptide sequences which can be incorporated into an activatable IL-2 polypeptide as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty (PCT) Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, and/or Chen et. al., J Bio Chem, 277, V6 P4485-4491. (2002), each of which is hereby incorporated by reference as if set forth herein in its entirety. In some embodiments, the cleavable peptide is one described in WO2024150172A1, the contents of which are incorporated by reference as if set forth herein in its entirety.

### Masked IL-2 Polypeptides and Anti-IL-2 Masks

In some embodiments, an IL-2 polypeptide of an immunocytokine composition (i.e., any one of the IL-2 polypeptides described herein) is masked in the immunocytokine composition by a masking polypeptide. In some embodiments, the masking polypeptide is one which specifically binds to the IL-2 polypeptide, thereby disrupting the ability of the IL-2 polypeptide to bind with its receptor.

In some embodiments, the masking polypeptide is selected from an anti-IL-2 antigen binding fragment, an IL-2 receptor subunit polypeptide, or a steric blocking group. In some embodiments, the masing polypeptide is an anti-IL-2 antigen binding fragment (or another anti-IL-2 binding domain, such as any of the binding domain formats described herein). In some embodiments, the masking polypeptide is an anti-IL-2 VHH or scFv. In some embodiments, the masking polypeptide is an anti-IL-2 scFv. In some embodiments, the masking polypeptide is an IL-2 receptor subunit polypeptide. In some embodiments, the IL-2 receptor subunit polypeptide is a binding sequence of an IL-2 receptor alpha subunit, and IL-2 receptor beta subunit, or an IL-2 receptor gamma subunit which is capable of binding to the IL-2 polypeptide (e.g., it acts as a “dummy receptor” of the IL-2 polypeptide). In some embodiments, the masking polypeptide is a steric blocking group (i.e., a bulky polypeptide that does not specifically interact with the IL-2 polypeptide but, by nature of its size, prevents or reduces binding of the IL-2 polypeptide with its receptor). In preferred embodiments, the masking polypeptide is an anti-IL-2 scFv.

In some preferred embodiments, the masking polypeptide (e.g., the anti-IL-2 scFv) is linked to the immunocytokine composition by a linker comprising a cleavable peptide (e.g., any of the cleavable peptide described herein, such as any of the protease cleavable peptide described herein). In some embodiments, the cleavable peptide is a protease cleavable peptide. In some embodiments, the cleavable peptide is cleavable by one or more proteases. In some embodiments, the cleavable peptide is cleavable by one or more proteases associated with a tumor or tumor microenvironment. In some embodiments, the cleavable peptide is preferentially or selectively cleaved in or near a tumor microenvironment. In some embodiments, the cleavable peptide is preferentially or selectively cleaved by one or more proteases associated with a tumor or tumor microenvironment.

In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by an MMP. In some embodiments, the cleavable peptide is cleavable by a matriptase. In some embodiments, the cleavable peptide is cleavable by a plasminogen activator. In some embodiments, the cleavable peptide is cleavable by a legumain. In some embodiments, the cleavable peptide is cleavable by a protease set forth in Table 6A.

In some embodiments, the cleavable peptide is cleavable by multiple proteases. In some embodiments, the cleavable peptide is cleavable by multiple classes of proteases. In some embodiments, the cleavable peptide is cleavable by 2, 3, or 4 different proteases. In some embodiments, the cleavable peptide comprises multiple cleavage sites. In some embodiments, the cleavable peptide comprises 2, 3, 4, or more cleavage sites. In some embodiments, the cleavable peptide comprises 2 cleavage sites. In some embodiments, the cleavable peptide comprises 3 cleavage sites. In some embodiments, the cleavable peptide comprises 4 cleavage sites. In some embodiments, each of the cleavage sites is cleavable by a different protease.

In some embodiments, the cleavable peptide is cleavable by a matrix metalloprotease and a legumain. In some embodiments, the cleavable peptide is cleavable by a matrix metalloprotease and a matriptase. In some embodiments, the cleavable peptide is cleavable by a matrix metalloprotease and a plasminogen activator. In some embodiments, the cleavable peptide is cleavable by a legumain and a matriptase. In some embodiments, the cleavable peptide is cleavable by a legumain and a plasminogen activator. In some embodiments, the cleavable peptide is cleavable by a matriptase and a plasminogen activator. In some embodiments, the cleavable peptide is cleavable by a matrix metalloprotease, a legumain, and a matriptase. In some embodiments, the cleavable peptide is cleavable by a matrix metalloprotease, a matriptase, and a plasminogen activator. In some embodiments, the cleavable peptide is cleavable by a matrix metalloprotease, a legumain, and a plasminogen activator.

Exemplary cleavable peptide sequences which can be incorporated into an activatable IL-2 polypeptide as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty (PCT) Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, WO2021/202678, and/or Chen et. al., J Bio Chem, 277, V6 P4485-4491. (2002), each of which is hereby incorporated by reference as if set forth herein in its entirety. In some embodiments, the cleavable peptide is one described in WO2024150172A1, the contents of which are incorporated by reference as if set forth herein in its entirety.

In some embodiments, cleavage of the cleavable peptide releases the masking polypeptide from the immunocytokine composition, thereby dissociating the masking polypeptide from the immunocytokine composition and allowing the IL-2 polypeptide to exhibit its activity and/or bind to its receptor.

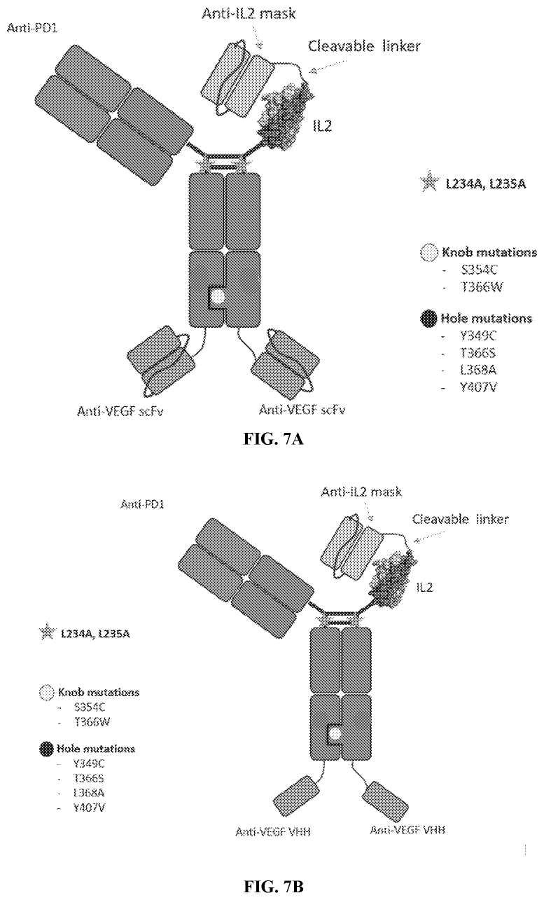

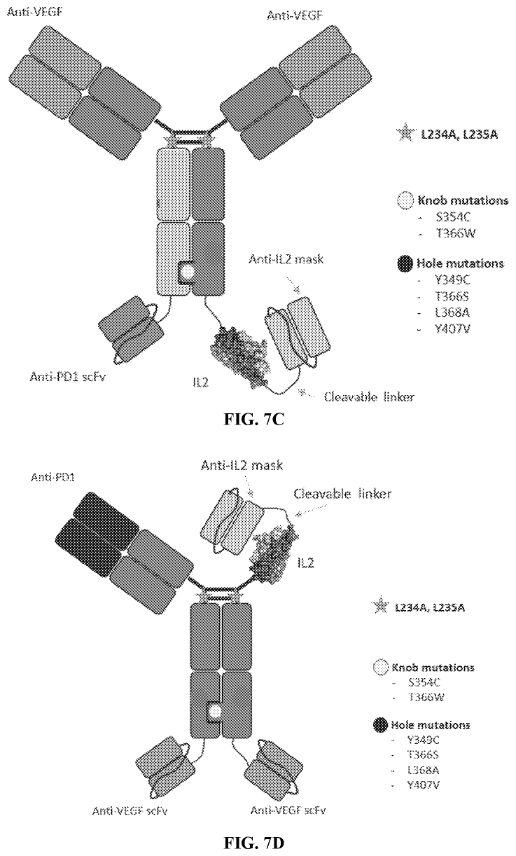

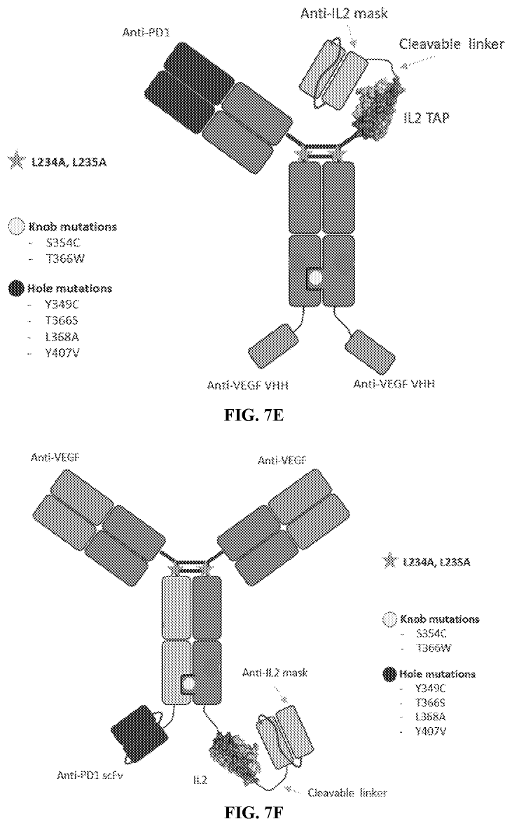

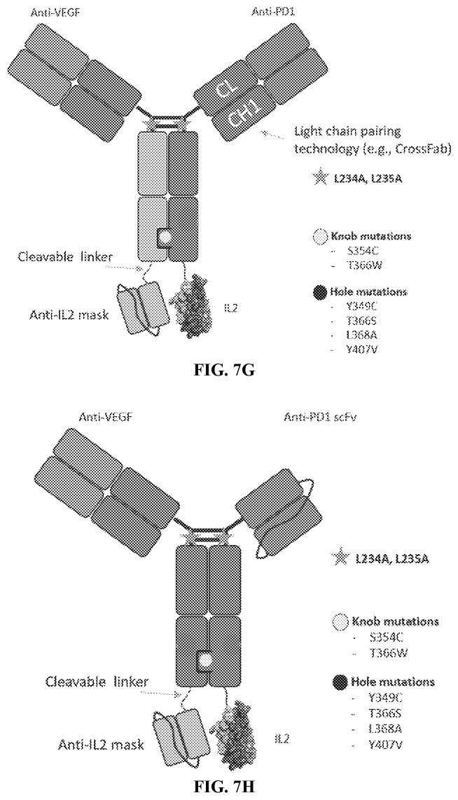

In some embodiments, the masking polypeptide (e.g., the anti-IL-2 scFv) is attached to the IL-2 polypeptide (e.g., it is fused to the N- or C-terminus of the IL-2 polypeptide via a linker which comprises a cleavable peptide). Examples of such immunocytokine compositions are shown in FIGS. 7A-7I. In some embodiments, the masking polypeptide (e.g., the anti-IL-2 scFv) is attached to the immunocytokine composition at a different location. In some embodiments, the masking polypeptide is attached to the immunocytokine composition at a different chain of the Fc domain from which the IL-2 polypeptide is attached.

Conjugation Vs. Fusion of IL-2 Polypeptide in Immunocytokine Compositions

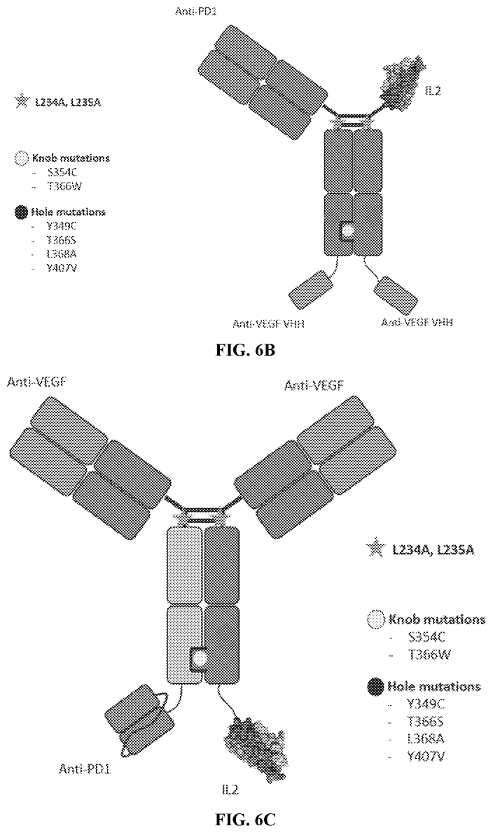

In some embodiments, an IL-2 polypeptide as described herein is attached to the immunocytokine composition via conjugation (i.e., by a conjugation reaction, such as with a conjugation handle). In some embodiments, an IL-2 polypeptide as described herein is fused to another portion of the immunocytokine composition. In general, it is preferable to conjugate chemically synthesized IL-2 polypeptides as described herein to form the covalent attachment (e.g., by using AJICAP technology to suitable derivatize an Fc domain), whereas for IL-2 polypeptides described herein which can be recombinantly produced, it may be preferable to incorporate them as a fusion protein (e.g., by fusing the IL-2 polypeptide to a polypeptide chain of an Fc domain, such as shown in FIGS. 6A-6C, 7A-7G, and 8A-8H). In some embodiments, the IL-2 polypeptide is fused via its C-terminus, optionally via a peptide linker. In some embodiments, the Il-2 polypeptide is fused via its N-terminus, optionally via a peptide linker.

Non-limiting examples of IL-2 polypeptides of the instant disclosure which can be linked into an immunocytokine composition described herein via conjugation include those provided in the table below.

TABLE 11
Sequences of Exemplary Conjugatable IL-2 Polypeptides.

| IL2 | | | |
|---|---|---|---|
| payload | | | SEQ ID |
| name | Substitutions | Sequence | NO |
| 2P72 | M23Nle, M39Nle, | APTSSSTKKTQLQLEHLLLDLQ-Nle- | 703 |
| | M46Nle, T41Hse, | ILNGINNYKNPKLTR-Nle-L-Hse-Y-KF-Y- | |
| | N71Hse, M104Hse, | Nle-PKKATELKHLQCLEEELKPLEEVL- | |
| | F42Y, C125S | Hse- | |
| | | LAQSKNFHLRPRDLISNINVIVLELKGSET | |
| | | TF-Hse- | |
| | | CEYADETATIVEFLNRWITFSQSIISTLT | |
| 2P73 | M23Nle, M39Nle, | APTSSSTKKTQLQLEHLLLDLQ-Nle- | 775 |
| | M46Nle, T41Hse, | ILNGINNHKNPRLTR-Nle-L-Hse-FKFY- | |
| | N71Hse, M104Hse, | NlePKKATELKHLQCLEEELKPLEEVL- | |
| | Y31H, K35R, Q74P, | HseLAPSKNFHLRPRDLISDINVIVLELKGS | |
| | N88D, C125S | ETTF-Hse- | |
| | | CEYADETATIVEFLNRWITFSQSIISTLT | |
| 2P74 | M23Nle, M39Nle, | APTSSSTKKTQLQLEHLLLDLQ-Nle- | 776 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | N88R, C125S, | Hse- | |
| | S130R | LAQSKNFHLRPRDLISRINVIVLELKGSET | |
| | | TF-Hse- | |
| | | CEYADETATIVEFLNRWITFSQSIIRTLT | |
| 2P75 | M23Nle, M39Nle, | APTSSSTKKTQLQLEHLLLDLQ-Nle- | 777 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | N88(Phe4COOH), | Hse-LAQSKNFHLRPRDLIS-Phe(4COOH)- | |
| | S130Orn | INVIVLELKGSETTF-Hse- | |
| | | CEYADETATIVEFLNRWITFSQSII-Orn- | |
| | | TLT | |
| 2P76 | M23Nle, M39Nle, | APTSSSTKKTQLQLE-Dab-LLDLQ-Nle- | 778 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | H16Dab, Del L17, | Hse-LAQSKNFHLRPRDLIS-Phe(4COOH)- | |
| | N88(Phe4COOH), | INVIVLELKGSETTF-Hse- | |
| | C125S, Q126Orn, | CEYADETATIVEFLNRWITFS-Orn-SII-Orn- | |
| | S130Orn | TLT | |
| 2P77 | M23Nle, M39Nle, | APTSSSTKKTQLQLE-Dab-LLLDLQ-Nle- | 779 |
| | M46Nle, T41Hse, | ILNGINNYKNPKLTR-Nle-L-Hse-Tyr(Bzl)- | |
| | N71Hse, M104Hse, | KFY-Nle- | |
| | H16Dab, F42(Tyr-O- | PKKATELKHLQCLEEELKPLEEVL-Hse- | |
| | Bzl), | LAQSKNFHLRPRDLIS-Phe(4COOH)- | |
| | N88(Phe4COOH), | INVIVLELKGSETTF-Hse- | |
| | C125S, Q126Orn, | CEYADETATIVEFLNRWITFS-Orn-SII-Orn- | |
| | S130Orn | TLT | |
| 2P78 | M23Nle, M39Nle, | APTSSSTKKTQLQLE-Dab-LLLDLQ-Nle- | 780 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | H16Dab, C125S, | Hse- | |
| | Q126Orn, S130Orn | LAQSKNFHLRPRDLISNINVIVLELKGSET | |
| | | TF-Hse-CEYADETATIVEFLNRWITFS-Orn- | |
| | | SII-Orn-TLT | |
| 2P79 | M23Nle, M39Nle, | APTSSSTKKTQLQLE-Dab-LLLDLQ-Nle- | 781 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | H16 Dab, | Hse-LAQSKNFHLRPRDLIS-Phe(4COOH)- | |
| | N88(Phe4COOH), | INVIVLELKGSETTF-Hse- | |
| | C125S, S130Orn | CEYADETATIVEFLNRWITFSQSII-Orn- | |
| | | TLT | |
| 2P80 | M23Nle, M39Nle, | APTSSSTKKTQLQLEHLLLDLQ-Nle- | 782 |
| | M46Nle, T41Hse, | ILNGINNYKNPKLTR-Nle-L-Hse-FKFY- | |
| | N71Hse, M104Hse, | Nle-PKKATELKHLQCLEEELKPLEEVL- | |
| | del(L80, R81, P82, | Hse- | |
| | R83), N88R, C125S, | LAQSKNFHDLISRINVIVLELKGSETTF- | |
| | S130R | Hse- | |
| | | CEYADETATIVEFLNRWITFSQSIIRTLT | |

In the table above Nle refers to a norleucine residue, Use refers to a homoserine residue, Phe(4 COOH) refers to a 4-carboxyphenylalanine residue, Dab refers to 2,4-diaminobutyric acid residue, Orn refers to an ornithine residue, and Tyr(Bzl) an G-benzyl-tyrosine residue.

In the table above, the listed SEQ ID NOs refer to the base sequences depicted in the table, and the “IL-2 Payload Name” refers to the conjugatable IL-2 polypeptide based on the corresponding SEQ ID NO. The structure of the conjugatable IL-2 polypeptide 2P72 is described supra. For each of conjugatable IL-2 polypeptides 2P73-2P80, the IL-2 polypeptide comprises an N-terminal modification of the structure:

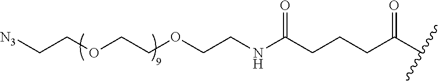

to provide a conjugation handle to the IL-2 polypeptide having the sequence noted in the SEQ ID NO listed in the table.

### Scaffolds for Multifunctional Immunocytokine Compositions

In some embodiments, the components of the immunocytokine composition (e.g., the anti-PD-1 binding domain, the anti-VEGFA binding domain, and the cytokine (e.g., the IL-2 polypeptide)) are all in covalent association. In some embodiments, each component is linked to the other portions of the immunocytokine composition via covalent bonds. In some embodiments, the portions of the immunocytokine composition are all linked to a scaffold group. Non-limiting examples of suitable scaffold groups include, for example, polypeptides (e.g., immunoglobulins or other biocompatible polypeptides), polymers (e.g., biocompatible polymers), particles (e.g., nanoparticles, microparticles, etc., such as those made from biocompatible polymers or metals), or any other such groups. In some preferred embodiments, all of the components are linked to a scaffold polypeptide, such as an immunoglobulin polypeptide. In some embodiments, one or more of the portions of the immunocytokine composition can be linked to the scaffold indirectly, such as through linkers (e.g., any of the linkers described herein, including peptide linkers for fusion proteins) or through other portions of the immunocytokine composition (e.g., the two binding domains are linked such that only one of the binding domains is attached to the scaffold (optionally through a suitable linker) and the other binding domain is linked the binding domain which is linked to the scaffold, independently and optionally though another linker). In some embodiments, all of the components are linked as a fusion protein of one or more polypeptides which combine to form the immunocytokine composition (e.g., each component is fused, directly or indirectly, to a polypeptide scaffold, such as an Fc domain).

### Fc Domains as Scaffolds

In some embodiments, the immunocytokine composition comprises an Fc domain which acts as a scaffold. In some embodiments, the Fc domain is fused to one, both, or each of the binding domains (or, for binding domains which comprise a plurality of polypeptide chains, such as a Fab, one of the polypeptide chains of the binding domain). In some embodiments, the Fc domain is also fused to the cytokine (e.g., the IL-2 polypeptide).

In some embodiments, the Fc domain is conjugated to the cytokine (e.g., the IL-2 polypeptide) via a linker. In some embodiments, such a linker is attached to a side chain of an amino acid residue of the Fc domain (e.g., a K246, K248, K288, K290, or K317 residue of the Fc domain (EU numbering), such as by AJICAP™ technology). Any of the Fc domains described herein (e.g., any one of SEQ ID NOs: 229-234 or 236-241, or a variant thereof) can comprise such a linker attached. In some embodiments, one arm of an Fc domain comprises a modification which prevents attachment of a linker at a specific residue, thereby facilitating conjugation of only a single group to the Fc domain scaffold (e.g., a substitution at one of residues K246, K248, K288, K290, or K317 on one arm of the Fc domain, thereby preventing attachment of the linker to that arm when using AJICAP™ technology). In some embodiments, the Fc domain comprises a K246A, K248A, K288A, K290A, or K317A substitution. In some embodiments, the Fc domain comprises a K248A substitution. Any of the Fc domains described herein (e.g., any one of SEQ ID NOs: 229-234 or 236-241, or a variant thereof) can comprise such a modification.

In some embodiments, an immunocytokine composition comprises an Fc domain comprising first CH2 and CH3 domains on a first polypeptide chain and second CH2 and CH3 domains on a second polypeptide chain.

In some embodiments, the immunocytokine composition comprises an Fc domain. In some embodiments, the Fc domain is an IgG Fc domain, an IgA Fc domain, an IgD Fc domain, an IgM Fc domain, or an IgE Fc domain, or a derivative thereof. In some embodiments, the Fc domain is an IgG Fc domain, an IgA Fc domain, or an IgD Fc domain, or a derivative thereof. In some embodiments, the Fc domain is a human Fc domain, or a derivative thereof. In some embodiments, the Fc domain is a humanized. Fc domain, or a derivative thereof. In some embodiments, the Fc domain is an IgG Fc domain, or a derivative thereof. In some instances, an IgG Fc domain is an IgG1 Fc domain, an IgG2a Fc domain, or an IgG4 Fc domain, or a derivative thereof. In some embodiments, the Fc domain is an IgG1 Fc domain, or a derivative thereof. In some embodiments, the Fc domain is an IgG4 Fc domain, or a derivative thereof. In some embodiments, a derivative of an Fc domain is one which contains at least 80%, 85%, 90%, 95%, 96%, 97%, 98%, or 99% sequence identity with a corresponding natural Fc domain (e.g., each arm has the indicated sequence identity).

In some embodiments, the Fc domain of an immunocytokine composition is an IgG1 Fc domain, or a derivative thereof. In some embodiments, the Fc domain comprises an amino acid sequence having at least 80%, 85%, 90%, 95%, 96%, 97%, 98%, 99%, or 100% sequence identity with a human IgG1 Fc domain (i.e., the CH2 and CH3 domains of human IgG1) (e.g., each arm of the Fc domain has the indicated sequence identity). In some embodiments, the Fc domain comprises an amino acid sequence at least 80%, 85%, 90%, 95%, 96%, 97%, 98%, 99%, or 100% sequence identity to a sequence set forth in the table below. In some embodiments, the Fc domain which comprises an amino acid sequence at least 80%, 85%, 90%, 95%, 96%, 97%, 98%, 99%, or 100% sequence identity to a sequence set forth in the table below can further comprise residues corresponding to residues 231-238 of an IgG1 (EU numbering) appended to the N-terminal residue of the sequences set forth in the table below (e.g., a peptide sequence of APELLGGP (SEQ ID NO: 227) (WT IgG1 “lower hinge”) or APEAAGGP (SEQ ID NO: 228) (LALA substituted “lower hinge”)). Such a sequence can also be considered to be co-extensive with the hinge region. Thus, when a dual binding composition described herein is described as having a hinge region described herein and a Fc domain as described herein, it is contemplated that the sequence of the hinge region described herein and the Fc domain described herein can contain overlap. In some embodiments, the Fc domain with the indicated sequence identity to the sequence set forth in the table below retains the noted substitutions (all of which are numbered with EU numbering). In some embodiments, the Fc domain comprises one or more additional substitutions described herein (e.g., K248A on one arm, etc.). In some embodiments, the Fc domain comprises one or more additional substitutions described herein (e.g., K248A on one arm, etc.) (e.g., 1, 2, 3, 4, 5, 6, 7, 8, 9, or more additional substitutions).

TABLE 12
Exemplary Fc Sequences

| | SEQ | |
|---|---|---|
| Description | ID NO | Sequence |
| IgG1 Fc Domain | 229 | SVFLFPPKPKDTLMISRTPEVTCVVVDVSH |
| (G1m1 allotype, | | EDPEVKFNWYVDGVEVHNAKTKPREEQY |
| includes E356D | | NSTYRVVSVLTVLHQDWLNGKEYKCKVS |
| and M358L) | | NKALPAPIEKTISKAKGQPREPQVYTLPPSR |
| (beginning with | | DELTKNQVSLTCLVKGFYPSDIAVEWESNG |
| Ser239 (EU | | QPENNYKTTPPVLDSDGSFFLYSKLTVDKS |
| numbering)) | | RWQQGNVFSCSVMHEALHNHYTQKSLSLS |
| | | PGK |
| IgG1 Fc Domain | 230 | SVFLFPPKPKDTLMISRTPEVTCVVVDVSHEDPEVK |
| with E356 and | | FNWYVDGVEVHNAKTKPREEQYNSTYRVVSVLTV |
| M358 (beginning | | LHQDWLNGKEYKCKVSNKALPAPIEKTISKAKGQP |
| with Ser239 (EU | | REPQVYTLPPSREEMTKNQVSLTCLVKGFYPSDIAV |
| numbering)) | | EWESNGQPENNYKTTPPVLDSDGSFFLYSKLTVDKS |
| | | RWQQGNVFSCSVMHEALHNHYTQKSLSLSPGK |
| IgG1 Fc Domain | 231 | SVFLFPPKPKDTLMISRTPEVTCVVVDVSHEDPEVK |
| with E356D and | | FNWYVDGVEVHNAKTKPREEQYNSTYRVVSVLTV |
| M358L | | LHQDWLNGKEYKCKVSNKALPAPIEKTISKAKGQP |
| substitutions and | | REPQVCTLPPSRDELTKNQVSLSCAVKGFYPSDIAV |
| Y349C, T366S, | | EWESNGQPENNYKTTPPVLDSDGSFFLVSKLTVDKS |
| L368A, and | | RWQQGNVFSCSVMHEALHNHYTQKSLSLSPGK |
| Y407V (hole | | |
| substitutions) | | |
| (beginning with | | |
| Ser239 (EU | | |
| numbering)) | | |
| IgG1 Fc domain | 232 | SVFLFPPKPKDTLMISRTPEVTCVVVDVSHEDPEVK |
| with E356D and | | FNWYVDGVEVHNAKTKPREEQYNSTYRVVSVLTV |
| M358L | | LHQDWLNGKEYKCKVSNKALPAPIEKTISKAKGQP |
| substitutions and | | REPQVYTLPPCRDELTKNQVSLWCLVKGFYPSDIA |
| S354C, T366W | | VEWESNGQPENNYKTTPPVLDSDGSFFLYSKLTVD |
| (knob | | KSRWQQGNVFSCSVMHEALHNHYTQKSLSLSPGK |
| substitutions) | | |
| (beginning with | | |
| Ser239 (EU | | |
| numbering)) | | |
| IgG1 Fc Domain | 233 | SVFLFPPKPKDTLMISRTPEVTCVVVDVSHEDPEVK |
| with E356 and | | FNWYVDGVEVHNAKTKPREEQYNSTYRVVSVLTV |
| M358 and | | LHQDWLNGKEYKCKVSNKALPAPIEKTISKAKGQP |
| Y349C, T366S, | | REPQVCTLPPSREEMTKNQVSLSCAVKGFYPSDIAV |
| L368A, and | | EWESNGQPENNYKTTPPVLDSDGSFFLVSKLTVDKS |
| Y407V (hole | | RWQQGNVFSCSVMHEALHNHYTQKSLSLSPGK |
| substitutions) | | |
| (beginning with | | |
| Ser239 (EU | | |
| numbering)) | | |
| IgG1 Fc domain | 234 | SVFLFPPKPKDTLMISRTPEVTCVVVDVSHEDPEVK |
| with E356 and | | FNWYVDGVEVHNAKTKPREEQYNSTYRVVSVLTV |
| M358 and S354C, | | LHQDWLNGKEYKCKVSNKALPAPIEKTISKAKGQP |
| T366W (knob | | REPQVYTLPPCREEMTKNQVSLWCLVKGFYPSDIA |
| substitutions) | | VEWESNGQPENNYKTTPPVLDSDGSFFLYSKLTVD |
| (beginning with | | KSRWQQGNVFSCSVMHEALHNHYTQKSLSLSPGK |
| Ser239 (EU | | |
| numbering)) | | |

In some embodiments, the Fc domain of an immunocytokine composition is an IgG4 Fc domain, or a derivative thereof. In some embodiments, the Fc domain comprises an amino acid sequence having at least 80%, 85%, 90%, 95%, 96%, 97%, 98%, 99%, or 100% sequence identity with a human IgG4 Fc domain (i.e., the CH2 and CH3 domains of human IgG4) (e.g., each arm of the Fc domain has the indicated sequence identity). In some embodiments, the Fc domain comprises an amino acid sequence at least 80%, 85%, 90%, 95%, 96%, 97%, 98%, 99%, or 100% sequence identity to a sequence set forth in the table below. In some embodiments, the Fc domain comprises an amino acid sequence at least 80%, 85%, 90%, 95%, 96%, 97%, 98%, 99%, or 100% sequence identity to a sequence set forth in the table below in which the first 8 residues of the sequences set forth below are omitted (e.g., the sequence APEFLGGP (SEQ ID NO: 235) is omitted). Such a sequence can also be considered to be co-extensive with the hinge region. Thus, when a dual binding composition described herein is described as having a hinge region described herein and a Fc domain as described herein, it is contemplated that the sequence of the hinge region described herein and the Fc domain described herein can contain overlap (i.e., when a dual binding composition is described as having an Fc domain described herein and a hinge region described herein, any overlapping portions of such sequences can be attributed to both regions). In some embodiments, the Fc domain with the indicated sequence identity to the sequence set forth in the table below retains the noted substitutions (all of which are numbered with EU numbering). In some embodiments, the Fc domain comprises one or more additional substitutions described herein (e.g., K248A on one arm, etc.) (e.g., 1, 2, 3, 4, 5, 6, 7, 8, 9, or more additional substitutions).

TABLE 13
Exemplary Fc Sequences

| | SEQ | |
|---|---|---|
| Description | ID NO | Sequence |
| Fc Domain | 236 | APEFLGGPSVFLFPPKPKDTLMISRTPEVTCVVVDVSQ |
| (IgG4) | | EDPEVQFNWYVDGVEVHNAKTKPREEQFNSTYRVVS |
| | | VLTVLHQDWLNGKEYKCKVSNKGLPSSIEKTISKAKG |
| | | QPREPQVYTLPPSQEEMTKNQVSLTCLVKGFYPSDIA |
| | | VEWESNGQPENNYKTTPPVLDSDGSFFLYSRLTVDKS |
| | | RWQEGNVFSCSVMHEALHNHYTQKSLSLSLGK |
| Fc Domain | 237 | APEFLGGPSVFLFPPKPKDTLMISRTPEVTCVVVDVSQ |
| with Y349C, | | EDPEVQFNWYVDGVEVHNAKTKPREEQFNSTYRVVS |
| T366S, L368A, | | VLTVLHQDWLNGKEYKCKVSNKGLPSSIEKTISKAKG |
| and Y407V | | QPREPQVCTLPPSQEEMTKNQVSLSCAVKGFYPSDIA |
| “Hole” | | VEWESNGQPENNYKTTPPVLDSDGSFFLVSRLTVDKS |
| mutations | | RWQEGNVFSCSVMHEALHNRFTQKSLSLSLGK |
| and H435R | | |
| and Y436F | | |
| “RF” | | |
| mutations | | |
| (IgG4) | | |
| Fc domain | 238 | APEFLGGPSVFLFPPKPKDTLMISRTPEVTCVVVDVSQ |
| with T366S, | | EDPEVQFNWYVDGVEVHNAKTKPREEQFNSTYRVVS |
| L368A, Y407V | | VLTVLHQDWLNGKEYKCKVSNKGLPSSIEKTISKAKG |
| substitutions | | QPREPQVYTLPPSQEEMTKNQVSLSCAVKGFYPSDIA |
| (IgG4) | | VEWESNGQPENNYKTTPPVLDSDGSFFLVSRLTVDKS |
| | | RWQEGNVFSCSVMHEALHNHYTQKSLSLSLGK |
| Fc Domain | 239 | APEFLGGPSVFLFPPKPKDTLMISRTPEVTCVVVDVSQ |
| with S354C | | EDPEVQFNWYVDGVEVHNAKTKPREEQFNSTYRVVS |
| and T366W | | VLTVLHQDWLNGKEYKCKVSNKGLPSSIEKTISKAKG |
| “Knob” | | QPREPQVYTLPPCQEEMTKNQVSLWCLVKGFYPSDIA |
| mutations | | VEWESNGQPENNYKTTPPVLDSDGSFFLYSRLTVDKS |
| (IgG4) | | RWQEGNVFSCSVMHEALHNHYTQKSLSLSLGK |
| Fc Domain | 240 | APEFLGGPSVFLFPPKPKDTLMISRTPEVTCVVVDVSQ |
| with T366W, | | EDPEVQFNWYVDGVEVHNAKTKPREEQFNSTYRVVS |
| H435R, and | | VLTVLHQDWLNGKEYKCKVSNKGLPSSIEKTISKAKG |
| Y436F | | QPREPQVYTLPPSQEEMTKNQVSLWCLVKGFYPSDIA |
| substitutions | | VEWESNGQPENNYKTTPPVLDSDGSFFLYSRLTVDKS |
| (IgG4) | | RWQEGNVFSCSVMHEALHNRFTQKSLSLSLGK |
| Fc Domain | 241 | APEFLGGPSVFLFPPKPKDTLMISRTPEVTCVVVDVSQ |
| with | | EDPEVQFNWYVDGVEVHNAKTKPREEQFNSTYRVVS |
| T366W | | VLTVLHQDWLNGKEYKCKVSNKGLPSSIEKTISKAKG |
| substitution | | QPREPQVYTLPPSQEEMTKNQVSLWCLVKGFYPSDIA |
| (IgG4) | | VEWESNGQPENNYKTTPPVLDSDGSFFLYSRLTVDKS |
| | | RWQEGNVFSCSVMHEALHNHYTQKSLSLSLGK |

One or more mutations may be introduced in an Fc domain to reduce Fe-mediated effector functions of immunocytokine composition, such as, for example, antibody-dependent cellular cytotoxicity (ADCC) and/or complement function. In some instances, a modified Fe comprises a humanized IgG4 kappa isotype that contains a S229P Fe mutation. In some instances, a modified Fe comprises a human IgG1 kappa where the heavy chain CH2 domain is engineered with a triple mutation such as, for example: (a) L238P, L239E, and P335S; or (2) K248; K288; and K317.

In some embodiments, the Fc domain comprises one or more modifications which favors heterodimerization of the two polypeptide which dimerize to form the Fe domains. Many such modifications are known in art for generating bispecific antibodies which can be applied to the instant disclosure. Such modifications are described in, for example, “Fc Engineering for Developing Therapeutic Bispecific Antibodies and Novel Scaffolds,” Liu et al., Front. Immunol., 26 Jan. 2017 (doi.org/10.3389/fimmu.2017.00038) and include, for example, knob-into-hole technology (see, e.g., U.S. Pat. No. 8,216,805) and modification introduced into one Fc domain to abrogate binding to protein A to facilitate purification of desired heterodimeric formats (e.g., RF mutations, as described in, e.g., U.S. Pat. No. 11,168,111). In some embodiments, the Fc domain of the immunocytokine compositions provided herein utilize knob-into-hole technology, for example the “hole” modifications of Y349C, T366S, L368A, and Y407V and the “knob” modifications of S354C and T366W (EU numbering). In some embodiments, the immunocytokine compositions provided herein utilize the RF mutations, e.g., H435R and Y436F mutations (EU numbering). In some embodiments, the immunocytokine compositions utilize both of these modifications together (e.g., one arm of the Fc domain of the immunocytokine composition having the hole and RF modifications, and one arm of the Fc domain of the immunocytokine composition having the knob modifications). In some embodiments, on arm of the Fc domain comprises T336W, H435R, and Y436F substitutions and the other arm of the Fc domain comprises T366S, L368A, Y407V substitutions. In some embodiments, one arm of the Fc domain comprises Y349C, T366S, L368A, and Y407V “hole” mutations and the other arm of the Fc domain comprises S354C and T366W “knob” mutations (EU numbering). Other combinations of such knob and hole modifications are well known in the art and compatible with the instant disclosure.

In some embodiments, one arm of the Fc domain of the immunocytokine composition comprises a mutation at a residue which eliminates the ability to conjugate an additional group (such as a cytokine, in particular an IL-2 polypeptide as described herein) to that site, thereby allowing for easier preparation of an immunocytokine composition which contains only a single cytokine (e.g., only one IL-2 polypeptide as described herein). For example, in instances where AJICAP™ technology is intended to be used to conjugate the cytokine (e.g., the IL-2 polypeptide) to one of residues K246, K248, K288, K290, or K317 (EU numbering), one arm of the Fc domain can comprise a mutation at the lysine residue to be targeted for conjugation to render it unavailable for reaction with the affinity peptide of the AJICAP™ technology. In one particular example, where it is intended to use an AJICAP™ affinity peptide to add a sulfide group to K248 for subsequent conjugation to the cytokine (e.g., through an intermediate reaction with a heterobifunctional linking reagent described herein), the K248 residue of one arm of the Fc domain is mutated to a suitable residue which is incapable of reaction with the affinity peptide, such as a K248A substitution. Thus, in some embodiments, one arm of the Fc domain contains a substitution at one or more of residue K246, K248, K288, K290, or K317. In some embodiments, the substitution is one or more of a K246A, K248A, K288A, K290A, or K317A substitution. In some embodiments, one arm of the Fc domain contains a K248A substitution. In some embodiments, one arm of the Fc domain contains a K248A substitution and the other arm is conjugated to the cytokine (e.g., the IL-2 polypeptide). Analogously, mutations at the other residues on one arm of the Fc domain can similarly be paired with conjugation at the corresponding unsubstituted residue on the other arm of the Fc domain (e.g., one arm with a K246A substitution and the other arm conjugated to the cytokine). Such substitutions to facilitate conjugation to only one arm of the Fc domain are favorably paired with substitutions which help to facilitate the heterodimerization of the arms of the Fc domain (e.g., knob-into-hole modifications discussed above or other similar modifications known in the art).

In some embodiments, the constant domains (e.g., the Fc domain) of an immunocytokine composition described herein can comprise further modifications (either in place of or in addition to the other modifications described herein, such as those which favor heterodimerization of two different arms of the fusion immunocytokine). Such modifications to Fc domains are known in the art and include, for example, modifications which alter antibody effector functions (e.g., enhance or decease Fc receptor binding or activity, thereby altering antibody-dependent cellular cytotoxicity, complement dependent cytotoxicity, or other effects), improve half-life circulation, or otherwise alter the performance of the molecule. Such modifications are well known in the art and are described in, for example, “Conceptual Approaches to Modulating Antibody Effector Functions and Circulation Half-Life,” Saunders et al., Fron. Immunol., 7 Jun. 2019 (doi.org/10.3389/fimmu.2019.01296). Such modifications can be at any relevant portion of the fusion immunocytokine, including without limitation an Fc domain (e.g., either the CH2 or CH3 domain, or both), a hinge region, a CH1 domain, a light chain constant region, and/or a framework region of an antigen binding domain (e.g., a VH or VL domain).

### Conjugation to Fc Domains

In some embodiments, the immunocytokine compositions described herein comprise an Fc domain, wherein the Fc domain comprises at least one covalently linked linker to a side chain of the Fc domain. In some embodiments, the linker is a chemical linker. In some embodiments, the chemical linker is covalently attached to a tyrosine, aspartic acid, glutamic acid, arginine, histidine, or lysine residue. In some embodiments, the chemical linker is covalently attached to a lysine, cysteine, or tyrosine residue. In some embodiments, the chemical linker is covalently attached to a cysteine residue. In some embodiments, the chemical linker is covalently attached to a lysine residue. In some embodiments, such a linker attaches the cytokine (e.g., the IL-2 polypeptide) to the Fc domain.

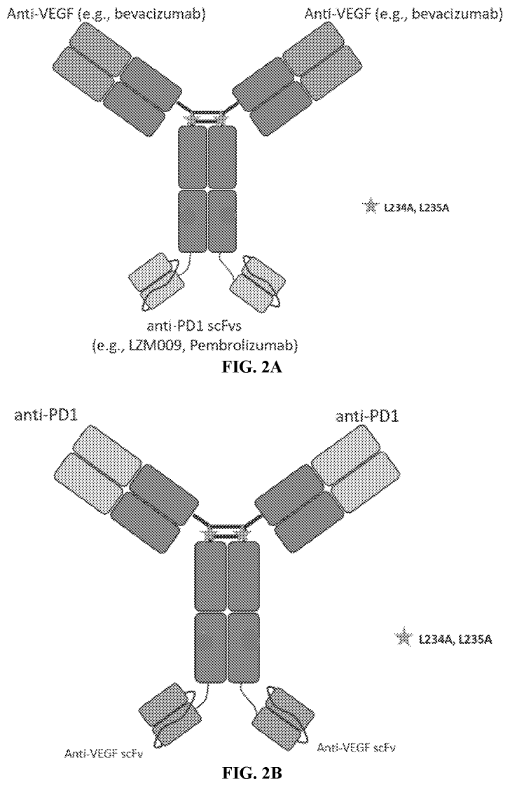

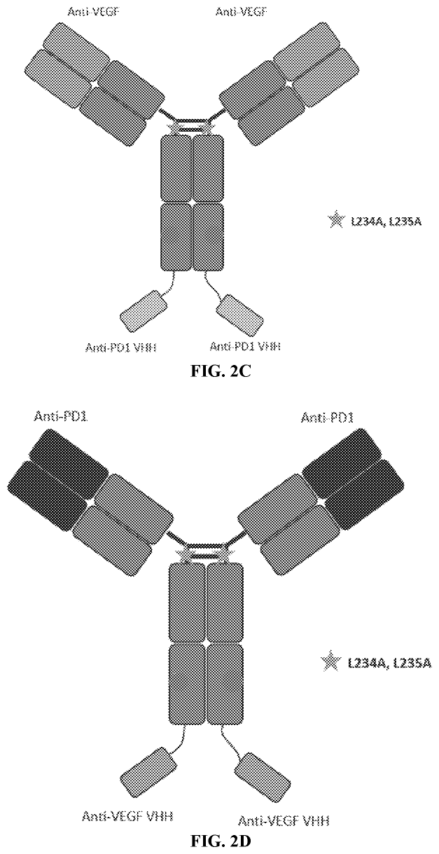

In some embodiments, the immunocytokine composition comprises one or both of the anti-PD-1 and/or the anti-VEGF binding domains fused to an Fc scaffold to which the cytokine (e.g., the IL-2 polypeptide) can be added by conjugation. Non-limiting examples of Fc domain containing scaffolds fused to anti-PD-1 and/or anti-VEGF binding domains suitable for addition of the cytokine (e.g., the IL-2 polypeptide) to form the immunocytokine composition are depicted in FIGS. 2A-D, 3A-D, 4A-C, and 5A-B. Non-limiting examples of sequences of such Fc domain scaffolds fused to anti-VEGF and anti-PD-1 binding domains can be found in the table below. In some embodiments, a cytokine of the instant disclosure (e.g., an IL-2 polypeptide described herein) is conjugated to one of the Fc-domain containing polypeptides below, such as by use of AJICAP™ technology to add a sulfide conjugation handle to a K246, K248, K288, K290, or K317 residue of the Fc domain (EU numbering). In some embodiments, a cytokine of the instant disclosure (e.g., an IL-2 polypeptide described herein) is fused to one of the Fc-domain containing polypeptides below, such by, for example, by replacing a VH and CH1 domain from a sequence below with the cytokine.

TABLE 14
Exemplary Bispecific Constructs Targeting VEGF and PD-1

| Notes | Chain | Sequence |
|---|---|---|
| Ivonescimab | HC | EVQLVESGGGLVQPGGSLRLSCAASGYTFTNYGMNWVRQAPGK |
| | | GLEWVGWINTYTGEPTYAADFKRRFTFSLDTSKSTAYLQMNSLR |
| | | AEDTAVYYCAKYPHYYGSSHWYFDVWGQGTLVTVSSASTKGPS |
| | | VFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEVQLVESGGGLVQ |
| | | PGGSLRLSCAASGFAFSSYDMSWVRQAPGKGLDWVATISGGGRY |
| | | TYYPDSVKGRFTISRDNSKNNLYLQMNSLRAEDTALYYCANRYG |
| | | EAWFAYWGQGTLVTVSSGGGGSGGGGSGGGGSGGGGSDIQMT |
| | | QSPSSMSASVGDRVTFTCRASQDINTYLSWFQQKPGKSPKTLIYR |
| | | ANRLVSGVPSRFSGSGSGQDYTLTISSLQPEDMATYYCLQYDEFP |
| | | LTFGAGTKLELKR (SEQ ID NO: 242) |
| | LC | DIQMTQSPSSLSASVGDRVTITCSASQDISNYLNWYQQKPGKAPK |
| | | VLIYFTSSLHSGVPSRFSGSGSGTDFTLTISSLQPEDFATYYCQQYS |
| | | TVPWTFGQGTKVEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC (SEQ ID NO: |
| | | 126) |
| Flipped | HC | EVQLVESGGGLVQPGGSLRLSCAASGFAFSSYDMSWVRQAPGKG |
| Ivonescimab | | LDWVATISGGGRYTYYPDSVKGRFTISRDNSKNNLYLQMNSLRA |
| (VEGF | | EDTALYYCANRYGEAWFAYWGQGTLVTVSSASTKGPSVFPLAPS |
| scFV VH- | | SKSTSGGTAALGCLVKDYFPEPVTVSWNSGALTSGVHTFPAVLQ |
| VL in bold) | | SSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLPP |
| | | SRDELTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGKGGGGSGGGGSGGGGSGGGGSEVQLVESGGGLVQPGGS |
| | | LRLSCAASGYTFTNYGMNWVRQAPGKGLEWVGWINTYTGE |
| | | PTYAADFKRRFTFSLDTSKSTAYLQMNSLRAEDTAVYYCAKY |
| | | PHYYGSSHWYFDVWGQGTLVTVSSGGGGSGGGGSGGGGSG |
| | | GGGSDIQMTQSPSSLSASVGDRVTITCSASQDISNYLNWYQQK |
| | | PGKAPKVLIYFTSSLHSGVPSRFSGSGSGTDFTLTISSLQPEDFA |
| | | TYYCQQYSTVPWTFGQGTKVEIK (SEQ ID NO: 243) |
| | LC | EIVLTQSPATLSLSPGERATISCRASKGVSTSGYSYLHWYQQKPG |
| | | QAPRLLIYLASYLESGVPARFSGSGSGTDFTLTISSLEPEDFATYYC |
| | | QHSRELPLTFGTGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC (SEQ ID |
| | | NO: 107) |
| Flipped | HC | EVQLVESGGGLVQPGGSLRLSCAASGFAFSSYDMSWVRQAPGKG |
| Ivonescimab | | LDWVATISGGGRYTYYPDSVKGRFTISRDNSKNNLYLQMNSLRA |
| (VEGF | | EDTALYYCANRYGEAWFAYWGQGTLVTVSSASTKGPSVFPLAPS |
| scFV VL- | | SKSTSGGTAALGCLVKDYFPEPVTVSWNSGALTSGVHTFPAVLQ |
| VH in bold) | | SSGLYSLSSVVTVPSSSLGTQTYICNVNHKPSNTKVDKKVEPKSC |
| | | DKTHTCPPCPAPEAAGGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKALPAPIEKTISKAKGQPREPQVYTLPP |
| | | SRDELTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGKGGGGSGGGGSGGGGSGGGGSDIQMTQSPSSLSASVGDR |
| | | VTITCSASQDISNYLNWYQQKPGKAPKVLIYFTSSLHSGVPSRF |
| | | SGSGSGTDFTLTISSLQPEDFATYYCQQYSTVPWTFGQGTKVE |
| | | IKGGGGSGGGGSGGGGSGGGGSEVQLVESGGGLVQPGGSLR |
| | | LSCAASGYTFTNYGMNWVRQAPGKGLEWVGWINTYTGEPT |
| | | YAADFKRRFTFSLDTSKSTAYLQMNSLRAEDTAVYYCAKYPH |
| | | YYGSSHWYFDVWGQGTLVTVSS (SEQ ID NO: 244) |
| | LC | SEQ ID NO: 107 |
| Bevacizumab | HC1 | EVQLVESGGGLVQPGGSLRLSCAASGYTFTNYGMNWVRQAPGK |
| Fab- | | GLEWVGWINTYTGEPTYAADFKRRFTFSLDTSKSTAYLQMNSLR |
| pembrolizumab | | AEDTAVYYCAKYPHYYGSSHWYFDVWGQGTLVTVSSASTKGPS |
| scFv | | VFPLAPSSKSTSGGTAALGCLVKDYFPEPVTVSWNSGALTSGVHT |
| VH-VL in | | FPAVLQSSGLYSLSSVVTVPSSSLGTQTYICNVNHKPSNTKVDKK |
| bold | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSQVQLVQSGVEVK |
| | | KPGASVKVSCKASGYTFTNYYMYWVRQAPGQGLEWMGGIN |
| | | PSNGGTNFNEKFKNRVTLTTDSSTTTAYMELKSLQFDDTAVY |
| | | YCARRDYRFDMGFDYWGQGTTVTVSSGGGGSGGGGSGGGG |
| | | SGGGGSEIVLTQSPATLSLSPGERATLSCRASKGVSTSGYSYLH |
| | | WYQQKPGQAPRLLIYLASYLESGVPARFSGSGSGTDFTLTISS |
| | | LEPEDFAVYYCQHSRDLPLTFGGGTKVEIK (SEQ ID NO: 245) |
| | LC | SEQ ID NO: 126 |
| Bevacizumab | HC1 | EVQLVESGGGLVQPGGSLRLSCAASGYTFTNYGMNWVRQAPGK |
| Fab- | | GLEWVGWINTYTGEPTYAADFKRRFTFSLDTSKSTAYLQMNSLR |
| pembrolizumab | | AEDTAVYYCAKYPHYYGSSHWYFDVWGQGTLVTVSSASTKGPS |
| scFv | | VFPLAPSSKSTSGGTAALGCLVKDYFPEPVTVSWNSGALTSGVHT |
| VL-VH in | | FPAVLQSSGLYSLSSVVTVPSSSLGTQTYICNVNHKPSNTKVDKK |
| bold | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEIVLTQSPATLSLS |
| | | PGERATLSCRASKGVSTSGYSYLHWYQQKPGQAPRLLIYLASYLE |
| | | SGVPARFSGSGSGTDFTLTISSLEPEDFAVYYCQHSRDLPLTFGGG |
| | | TKVEIKGGGGSGGGGSGGGGSGGGGSQVQLVQSGVEVKKPGAS |
| | | VKVSCKASGYTFTNYYMYWVRQAPGQGLEWMGGINPSNGGTN |
| | | FNEKFKNRVTLTTDSSTTTAYMELKSLQFDDTAVYYCARRDYRF |
| | | DMGFDYWGQGTTVTVSS (SEQ ID NO: 246) |
| | LC | SEQ ID NO: 126 |
| pembrolizumab | HC | QVQLVQSGVEVKKPGASVKVSCKASGYTFTNYYMYWVRQAPG |
| Fab- | | QGLEWMGGINPSNGGTNFNEKFKNRVTLTTDSSTTTAYMELKSL |
| bevacizumab | | QFDDTAVYYCARRDYRFDMGFDYWGQGTTVTVSSASTKGPSVF |
| scFv VH- | | PLAPSSKSTSGGTAALGCLVKDYFPEPVTVSWNSGALTSGVHTFP |
| VL in bold | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGLVQP |
| | | GGSLRLSCAASGYTFTNYGMNWVRQAPGKGLEWVGWINTY |
| | | TGEPTYAADFKRRFTFSLDTSKSTAYLQMNSLRAEDTAVYYC |
| | | AKYPHYYGSSHWYFDVWGQGTLVTVSSGGGGSGGGGSGGG |
| | | GSGGGGSDIQMTQSPSSLSASVGDRVTITCSASQDISNYLNWY |
| | | QQKPGKAPKVLIYFTSSLHSGVPSRFSGSGSGTDFTLTISSLQP |
| | | EDFATYYCQQYSTVPWTFGQGTKVEIK (SEQ ID NO: 247) |
| | LC | EIVLTQSPATLSLSPGERATLSCRASKGVSTSGYSYLHWYQQKPG |
| | | QAPRLLIYLASYLESGVPARFSGSGSGTDFTLTISSLEPEDFAVYYC |
| | | QHSRDLPLTFGGGTKVEIKRTVAAPSVFIFPPSDEQLKSGTASVVC |
| | | LLNNFYPREAKVQWKVDNALQSGNSQESVTEQDSKDSTYSLSST |
| | | LTLSKADYEKHKVYACEVTHQGLSSPVTKSFNRGEC (SEQ ID |
| | | NO: 47) |
| Pembrolizumab | HC | QVQLVQSGVEVKKPGASVKVSCKASGYTFTNYYMYWVRQAPG |
| Fab- | | QGLEWMGGINPSNGGTNFNEKFKNRVTLTTDSSTTTAYMELKSL |
| bevacizumab | | QFDDTAVYYCARRDYRFDMGFDYWGQGTTVTVSSASTKGPSVF |
| scFv VL- | | PLAPSSKSTSGGTAALGCLVKDYFPEPVTVSWNSGALTSGVHTFP |
| VH in bold | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSDIQMTQSPSSLSASV |
| | | GDRVTITCSASQDISNYLNWYQQKPGKAPKVLIYFTSSLHSGV |
| | | PSRFSGSGSGTDFTLTISSLQPEDFATYYCQQYSTVPWTFGQG |
| | | TKVEIKGGGGSGGGGSGGGGSGGGGSEVQLVESGGGLVQPG |
| | | GSLRLSCAASGYTFTNYGMNWVRQAPGKGLEWVGWINTYT |
| | | GEPTYAADFKRRFTFSLDTSKSTAYLQMNSLRAEDTAVYYCA |
| | | KYPHYYGSSHWYFDVWGQGTLVTVSS (SEQ ID NO: 248) |
| | LC | SEQ ID NO: 47 |
| Bevacizumab | HC | EVQLVESGGGLVQPGGSLRLSCAASGYTFTNYGMNWVRQAPGK |
| Fab- | | GLEWVGWINTYTGEPTYAADFKRRFTFSLDTSKSTAYLQMNSLR |
| LZM009 | | AEDTAVYYCAKYPHYYGSSHWYFDVWGQGTLVTVSSASTKGPS |
| scFv VH- | | VFPLAPSSKSTSGGTAALGCLVKDYFPEPVTVSWNSGALTSGVHT |
| VL in bold | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSQVQLVQSGAEVK |
| | | KPGASVKVSCKASGYTFTSYYMYWVRQAPGQGLEWMGGVN |
| | | PSNGGTNFNEKFKSRVTITADKSTSTAYMELSSLRSEDTAVYY |
| | | CARRDYRYDMGFDYWGQGTTVTVSSGGGGSGGGGSGGGGS |
| | | GGGGSEIVLTQSPATLSLSPGERATISCRASKGVSTSGYSYLH |
| | | WYQQKPGQAPRLLIYLASYLESGVPARFSGSGSGTDFTLTISS |
| | | LEPEDFATYYCQHSRELPLTFGTGTKVEIK (SEQ ID NO: 249) |
| | LC | SEQ ID NO: 126 |
| Bevacizumab | HC | EVQLVESGGGLVQPGGSLRLSCAASGYTFTNYGMNWVRQAPGK |
| Fab- | | GLEWVGWINTYTGEPTYAADFKRRFTFSLDTSKSTAYLQMNSLR |
| LZM009 | | AEDTAVYYCAKYPHYYGSSHWYFDVWGQGTLVTVSSASTKGPS |
| scFv VL- | | VFPLAPSSKSTSGGTAALGCLVKDYFPEPVTVSWNSGALTSGVHT |
| VH in bold | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEIVLTQSPATLSL |
| | | SPGERATISCRASKGVSTSGYSYLHWYQQKPGQAPRLLIYLAS |
| | | YLESGVPARFSGSGSGTDFTLTISSLEPEDFATYYCQHSRELPL |
| | | TFGTGTKVEIKGGGGSGGGGSGGGGSGGGGSQVQLVQSGAE |
| | | VKKPGASVKVSCKASGYTFTSYYMYWVRQAPGQGLEWMGG |
| | | VNPSNGGTNFNEKFKSRVTITADKSTSTAYMELSSLRSEDTAV |
| | | YYCARRDYRYDMGFDYWGQGTTVTVSS (SEQ ID NO: 250) |
| | LC | SEQ ID NO: 126 |
| LZM009 | HC | QVQLVQSGAEVKKPGASVKVSCKASGYTFTSYYMYWVRQAPG |
| Fab- | | QGLEWMGGVNPSNGGTNFNEKFKSRVTITADKSTSTAYMELSSL |
| bevacizumab | | RSEDTAVYYCARRDYRYDMGFDYWGQGTTVTVSSASTKGPSVF |
| scFv VH- | | PLAPSSKSTSGGTAALGCLVKDYFPEPVTVSWNSGALTSGVHTFP |
| VL in bold | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGLVQP |
| | | GGSLRLSCAASGYTFTNYGMNWVRQAPGKGLEWVGWINTY |
| | | TGEPTYAADFKRRFTFSLDTSKSTAYLQMNSLRAEDTAVYYC |
| | | AKYPHYYGSSHWYFDVWGQGTLVTVSSGGGGSGGGGSGGG |
| | | GSGGGGSDIQMTQSPSSLSASVGDRVTITCSASQDISNYLNWY |
| | | QQKPGKAPKVLIYFTSSLHSGVPSRFSGSGSGTDFTLTISSLQP |
| | | EDFATYYCQQYSTVPWTFGQGTKVEIK (SEQ ID NO: 251) |
| | LC | SEQ ID NO: 107 |
| LZM009 | HC | QVQLVQSGAEVKKPGASVKVSCKASGYTFTSYYMYWVRQAPG |
| Fab- | | QGLEWMGGVNPSNGGTNFNEKFKSRVTITADKSTSTAYMELSSL |
| bevacizumab | | RSEDTAVYYCARRDYRYDMGFDYWGQGTTVTVSSASTKGPSVF |
| scFv VL- | | PLAPSSKSTSGGTAALGCLVKDYFPEPVTVSWNSGALTSGVHTFP |
| VH in bold | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSDIQMTQSPSSLSASV |
| | | GDRVTITCSASQDISNYLNWYQQKPGKAPKVLIYFTSSLHSGV |
| | | PSRFSGSGSGTDFTLTISSLQPEDFATYYCQQYSTVPWTFGQG |
| | | TKVEIKGGGGSGGGGSGGGGSGGGGSEVQLVESGGGLVQPG |
| | | GSLRLSCAASGYTFTNYGMNWVRQAPGKGLEWVGWINTYT |
| | | GEPTYAADFKRRFTFSLDTSKSTAYLQMNSLRAEDTAVYYCA |
| | | KYPHYYGSSHWYFDVWGQGTLVTVSS (SEQ ID NO: 252) |
| | LC | SEQ ID NO: 107 |
| Bevacizumab | HC | EVQLVESGGGLVQPGGSLRLSCAASGYTFTNYGMNWVRQAPGK |
| Fab-anti- | | GLEWVGWINTYTGEPTYAADFKRRFTFSLDTSKSTAYLQMNSLR |
| PD1 | | AEDTAVYYCAKYPHYYGSSHWYFDVWGQGTLVTVSSASTKGPS |
| nanobody | | VFPLAPSSKSTSGGTAALGCLVKDYFPEPVTVSWNSGALTSGVHT |
| in bold | | FPAVLQSSGLYSLSSVVTVPSSSLGTQTYICNVNHKPSNTKVDKK |
| (VHH 4) | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEVQLVESGGGEV |
| | | QPGGSLRLSCAASGSITGANTMGWYRQAPGKQRDLVALIGN |
| | | YVTHYAESVKGRFTISRDNAKNTVYLQMSSLRAEDTAVYYCY |
| | | LYTDNLGTSWGQGTLVTVKP (SEQ ID NO: 264) |
| | LC | SEQ ID NO: 126 |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 X | | FPAVLQSSGLYSLSSVVTVPSSSLGTQTYICNVNHKPSNTKVDKK |
| scFv | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEVQLVESGGGLVQ |
| | | PGGSLRLSCAASGFAFSNYYMYWVRQAPGKGLEWMGGINPSNG |
| | | GTNFNEKFKNRVTISRDNSKNNLYLQMNSLRAEDTALYYCARRD |
| | | YRFDMGFDYWGQGTLVTVSSGGGGSGGGGSGGGGSGGGGSDIQ |
| | | MTQSPSSMSASVGDRVTFTCRASKGVSTSGYSYLHWFQQKPGKS |
| | | PKTLIYLASYLESGVPSRFSGSGSGQDYTLTISSLQPEDMATYYCQ |
| | | HSRDLPLTFGAGTKLELKR (SEQ ID NO: 253) |
| | LC | SEQ ID NO: 126 |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 X | | FPAVLQSSGLYSLSSVVTVPSSSLGTQTYICNVNHKPSNTKVDKK |
| scFv | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSDIQMTQSPSSMSA |
| | | SVGDRVTFTCRASKGVSTSGYSYLHWFQQKPGKSPKTLIYLASYL |
| | | ESGVPSRFSGSGSGQDYTLTISSLQPEDMATYYCQHSRDLPLTFG |
| | | AGTKLELKRGGGGSGGGGSGGGGSGGGGSEVQLVESGGGLVQP |
| | | GGSLRLSCAASGFAFSNYYMYWVRQAPGKGLEWMGGINPSNGG |
| | | TNFNEKFKNRVTISRDNSKNNLYLQMNSLRAEDTALYYCARRDY |
| | | RFDMGFDYWGQGTLVTVSS (SEQ ID NO: 254) |
| | LC | SEQ ID NO: 126 |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 Y | | FPAVLQSSGLYSLSSVVTVPSSSLGTQTYICNVNHKPSNTKVDKK |
| scFv | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEVQLVESGGGLVQ |
| | | PGGSLRLSCAASGFAFSSYYMYWVRQAPGKGLEWMGGVNPSNG |
| | | GTNFNEKFKSRVTISRDNSKNNLYLQMNSLRAEDTALYYCARRD |
| | | YRYDMGFDYWGQGTLVTVSSGGGGSGGGGSGGGGSGGGGSDIQ |
| | | MTQSPSSMSASVGDRVTFTCRASKGVSTSGYSYLHWFQQKPGKS |
| | | PKTLIYLASYLESGVPSRFSGSGSGQDYTLTISSLQPEDMATYYCQ |
| | | HSRELPLTFGAGTKLELKR (SEQ ID NO: 255) |
| | LC | SEQ ID NO: 126 |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 Y | | FPAVLQSSGLYSLSSVVTVPSSSLGTQTYICNVNHKPSNTKVDKK |
| scFv | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSDIQMTQSPSSMSA |
| | | SVGDRVTFTCRASKGVSTSGYSYLHWFQQKPGKSPKTLIYLASYL |
| | | ESGVPSRFSGSGSGQDYTLTISSLQPEDMATYYCQHSRELPLTFGA |
| | | GTKLELKRGGGGSGGGGSGGGGSGGGGSEVQLVESGGGLVQPG |
| | | GSLRLSCAASGFAFSSYYMYWVRQAPGKGLEWMGGVNPSNGGT |
| | | NFNEKFKSRVTISRDNSKNNLYLQMNSLRAEDTALYYCARRDYR |
| | | YDMGFDYWGQGTLVTVSS (SEQ ID NO: 256) |
| | LC | SEQ ID NO: 126 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH175 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSDVQLVESGGGLVQP |
| | | GGSLRLSCAASGRTFSSYSMGWFRQAPGKEREFVVAISKGGYKY |
| | | DAVSLEGRFTISRDNAKNTVYLQINSLRPEDTAVYYCASSRAYGS |
| | | SRLRLADTYEYWGQGTLVTVSS (SEQ ID NO: 258) |
| | LC | SEQ ID NO: 47 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH184 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGVVQP |
| | | GGSLRLSCAASGRTFSSYSMGWFRQAPGKGLEFVVAISKGGYKY |
| | | DAVSLEGRFTISRDNSKNTVYLQMNSLRAEDTAVYYCASSRAYG |
| | | SSRLRLADTYEYWGQGTLVTVSS (SEQ ID NO: 259) |
| | LC | SEQ ID NO: 47 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH185 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGVVQP |
| | | GGSLRLSCAASGRTFSSYSMGWFRQAPGKEREFVVAISKGGYKY |
| | | DAVSLEGRFTISRDNSKNTVYLQMNSLRAEDTAVYYCASSRAYG |
| | | SSRLRLADTYEYWGQGTLVTVSS (SEQ ID NO: 260) |
| | LC | SEQ ID NO: 47 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH186 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGLVQPG |
| | | GSLRLSCAASGRTFSSYSMGWFRQAPGKEREFVVAISKGGYKYD |
| | | AVSVKGRFTISRDNSKNTVYLQMGSLRAEDMAVYYCASSRAYG |
| | | SSRLRLADTYEYWGQGTLVTVSS (SEQ ID NO: 261) |
| | LC | SEQ ID NO: 47 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH187 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGLVQPG |
| | | GSLRLSCAASGRTFSSYSMGWFRQAPGKGLEFVVAISKGGYKYD |
| | | AVSVKGRFTISRDNSKNTVYLQMGSLRAEDMAVYYCASSRAYG |
| | | SSRLRLADTYEYWGQGTLVTVSS (SEQ ID NO: 262) |
| | LC | SEQ ID NO: 47 |
| Anti PD1 | HC | QVQLVQSGVEVKKPGASVKVSCKASGYTFTNYYMYWVRQAPG |
| Fab | | QGLEWMGGINPSNGGTNFNEKFKNRVTLTTDSSTTTAYMELKSL |
| (Pembrolizumab)- | | QFDDTAVYYCARRDYRFDMGFDYWGQGTTVTVSSASTKGPSVF |
| Fc- | | PLAPSSKSTSGGTAALGCLVKDYFPEPVTVSWNSGALTSGVHTFP |
| anti VEGF | | AVLQSSGLYSLSSVVTVPSSSLGTQTYICNVNHKPSNTKVDKKVE |
| VHH188 | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALPAPIEKTISKAKGQPREPQVY |
| | | TLPPSRDELTKNQVSLTCLVKGFYPSDIAVEWESNGQPENNYKTT |
| | | PPVLDSDGSFFLYSKLTVDKSRWQQGNVFSCSVMHEALHNHYTQ |
| | | KSLSLSPGKGGGGSGGGGSGGGGSGGGGSEVQLVESGGGLVQPG |
| | | GSLRLSCAASGRTFSSYSMGWFRQAPGKEREFVVAISKGGYKYD |
| | | AVSVKGRFTISRDNAKNTVYLQMNSLRAEDTAVYYCASSRAYGS |
| | | SRLRLADTYEYWGQGTLVTVSS (SEQ ID NO: 263) |
| | LC | SEQ ID NO: 47 |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 | | FPAVLQSSGLYSLSSVVTVPSSSLGTQTYICNVNHKPSNTKVDKK |
| VHH1 | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSDVQLVESGGGVV |
| | | QPGGSLRLSCAASGSIASIHAMGWFRQAPGKEREFVAVITWSGGI |
| | | TYYADSVKGRFTISRDNSKNTVYLQMNSLRPEDTALYYCAGDKH |
| | | QSSWYDYWGQGTLVTVSS (SEQ ID NO: 291) |
| | LC | SEQ ID NO: 126 |
| Anti VEGF | HC | SEQ ID NO: 291 |
| Fab | LC | DIQMTQSPSSLSASVGDRVTITCSASQDISNYLNWYQQKPGKAPK |
| (Bevacizumab)- | | VLIYFTSSLHSGVPSRFSGSGSGTDFTLTISSLQPEDFATYYCQQYS |
| Fc- | | TVPWTFGQGTKVEIKRTVAAPSVFIFPPSDEQLKSGTASVVCLLN |
| anti PD1 | | NFYPREAKVQWKVDNAKQSGNSQESVTEQDSKDSTYSLSSTLTL |
| VHH1 | | SKADYEKHKVYACEVTHQGLSSPVTKSFNRGEC (SEQ ID NO: |
| | | 285) |
| Anti VEGF | HC | SEQ ID NO: 291 |
| Fab | LC | DIQMTQSPSSLSASVGDRVTITCSASQDISNYLNWYQQKPGKAPK |
| (Bevacizumab)- | | VLIYFTSSLHSGVPSRFSGSGSGTDFTLTISSLQPEDFATYYCQQYS |
| Fc- | | TVPWTFGQGTKVEIKRTVAAPSVFIFPPSDEQLKSGTASVVCLLN |
| anti PD1 | | NFYPREAKVQWKVDNAKQSGNSQESVTEQDSKDSTYSLSSTLTL |
| VHH1 | | SKADYEKHKVYACEVTHQGKSSPVTKSFNRGEC (SEQ ID NO: |
| | | 286) |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 | | FPAVLQSSGLYSLSSVVTVPSSSLGTQTYICNVNHKPSNTKVDKK |
| VHH2 | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGGGGSGGGGSGGGGSGGGGSEVQLVESGGGLVKPG |
| | | GSLRLSCAASGFTFSDESMTWMRQAPGKGLEWVSYISSGGGVKF |
| | | YADSVKGRFTISRDNAKNSLYLQMNSLRAEDTAVYYCAREAPLR |
| | | LGESPHDAFDISGQGTMVTVSS (SEQ ID NO: 292) |
| | LC | SEQ ID NO: 126 |
| Anti VEGF Fab | HC | SEQ ID NO: 292 |
| (Bevacizumab)- | LC | SEQ ID NO: 285 |
| Fc-anti PD1 VHH2 | | |
| Anti VEGF Fab | HC | SEQ ID NO: 292 |
| (Bevacizumab)- | LC | SEQ ID NO: 286 |
| Fc-anti PD1 VHH2 | | |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 | | FPAVLQSSGLYSLSSVVTVPSSSLGTQTYICNVNHKPSNTKVDKK |
| VHH3 | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSQVQLQESGGGSV |
| | | QAGGSLRLSCVASQYTYNTVGWFRQAPGKEREGVAGIYNGGDQ |
| | | TYYSESAKGRFTISQDNAKRTVYLQMNSLKPEDTAMYYCAAGRL |
| | | IVSGRWSMTKEEYQYWGQGTQVTVSS (SEQ ID NO: 293) |
| | LC | SEQ ID NO: 126 |
| Anti VEGF Fab | HC | SEQ ID NO: 293 |
| (Bevacizumab)- | LC | SEQ ID NO: 285 |
| Fc-anti PD1 VHH3 | | |
| Anti VEGF Fab | HC | SEQ ID NO: 293 |
| (Bevacizumab)- | LC | SEQ ID NO: 286 |
| Fc-anti PD1 VHH3 | | |
| Anti VEGF Fab | HC | EVQLVESGGGLVQPGGSLRLSCAASGYTFTNYGMNWVRQAPGK |
| (Bevacizumab)- | | GLEWVGWINTYTGEPTYAADFKRRFTFSLDTSKSTAYLQMNSLR |
| Fc- | | AEDTAVYYCAKYPHYYGSSHWYFDVWGQGTLVTVSSASTKGPS |
| anti PD1 | | VFPLAPSSKSTSGGTAALGCLVKDYFPEPVTVSWNSGALTSGVHT |
| VHH4 | | FPAVLQSSGLYSLSSVVTVPSSSLGTQTYICNVNHKPSNTKVDKK |
| | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGKGGGGSGGGGSGGGGSGGGGSEVQLVESGGGEVQ |
| | | PGGSLRLSCAASGSITGANTMGWYRQAPGKQRDLVALIGNYVTH |
| | | YAESVKGRFTISRDNAKNTVYLQMSSLRAEDTAVYYCYLYTDNL |
| | | GTSWGQGTLVTVKP (SEQ ID NO: 264) |
| | LC | SEQ ID NO: 126 |
| Anti VEGF Fab | HC | SEQ ID NO: 264 |
| (Bevacizumab)- | LC | SEQ ID NO: 285 |
| Fc-anti PD1 VHH4 | | |
| Anti VEGF Fab | HC | SEQ ID NO: 264 |
| (Bevacizumab)- | LC | SEQ ID NO: 286 |
| Fc-anti PD1 VHH4 | | |
| Anti VEGF | HC | EVQLVESGGGLVQPGGSLRLSCAASGYTFTNYGMNWVRQAPGK |
| Fab | | GLEWVGWINTYTGEPTYAADFKRRFTFSLDTSKSTAYLQMNSLR |
| (Bevacizumab)- | | AEDTAVYYCAKYPHYYGSSHWYFDVWGQGTLVTVSSASTKGPS |
| Fc- | | VFPLAPSSKSTSGGTAALGCLVKDYFPEPVTVSWNSGALTSGVHT |
| anti PD1 | | FPAVLQSSGLYSLSSVVTVPSSSLGTQTYICNVNHKPSNTKVDKK |
| VHH5 | | VEPKSCDKTHTCPPCPAPEAAGGPSVFLFPPKPKDTLMISRTPEVT |
| | | CVVVDVSHEDPEVKFNWYVDGVEVHNAKTKPREEQYNSTYRVV |
| | | SVLTVLHQDWLNGKEYKCKVSNKALPAPIEKTISKAKGQPREPQ |
| | | VYTLPPSRDELTKNQVSLTCLVKGFYPSDIAVEWESNGQPENNYK |
| | | TTPPVLDSDGSFFLYSKLTVDKSRWQQGNVFSCSVMHEALHNHY |
| | | TQKSLSLSPGGGGGSGGGGSGGGGSGGGGSEVQLVESGGGLVQP |
| | | GGSLRLSCAVSGNIYNRNFMGWFRQAPGKGLEGVSAIYTGTSRT |
| | | YYADSVKGRFTISRDNSKNTVYLQMNSLRAEDTAVYYCAADLR |
| | | EGFWDTGVWNTWGQGTLVTVSS (SEQ ID NO: 257) |
| | LC | SEQ ID NO: 126 |
| Anti VEGF Fab | HC | SEQ ID NO: 257 |
| (Bevacizumab)- | LC | SEQ ID NO: 285 |
| Fc-anti PD1 VHH5 | | |
| Anti VEGF Fab | HC | SEQ ID NO: 257 |
| (Bevacizumab)- | LC | SEQ ID NO: 286 |
| Fc-anti PD1 VHH5 | | |

The chemical linker can be covalently attached to one amino acid residue of an Fc region of the immunocytokine composition. In some embodiments, the chemical linker is covalently attached to a non-terminal residue of the Fc region. In some embodiments, the non-terminal residue is in the CH1, CH2, or CH3 region of the immunocytokine composition. In some embodiments, the non-terminal residue is in the CH2 region of the immunocytokine composition.

In some embodiments, the chemical linker is covalently attached at an amino acid residue of the immunocytokine composition such that the function of a binding domain fused to the Fc domain is maintained (e.g., without denaturing the polypeptide). For example, when immunocytokine composition comprises a human IgG (e.g., human IgG1), exposed lysine residues and exposed tyrosine residues are present at the following positions (refer to web site www.imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Tyrosine Residues: CH2 domain (position 278, position 296, and position 300) CH3 domain (position 436).

The human IgG, such as human IgG1, may also be modified with a lysine or tyrosine residue at any one of the positions listed above in order provide a residue which is ideally surface exposed for subsequent modification.

In some embodiments, the chemical linker is covalently attached at an amino acid residue in the constant region of an immunocytokine composition. In some embodiments, the chemical linker is covalently attached at an amino acid residue in the CH1, CH2, or CH3 region. In some embodiments, the chemical linker is covalently attached at an amino acid residue in the CH2 region. In some embodiments, the chemical linker may be covalently attached to one amino acid residue in the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the chemical linker is covalently attached to one lysine residue of a human IgG Fc domain (e.g. IgG1 or IgG4). In some embodiments, the chemical linker is covalently attached at Lys 246, Lys 248, Lys 288, Lys 290, or Lys 317 of the Fc domain (EU numbering). In some embodiments, the chemical linker is covalently attached at Lys 246 of an Fc region of the Fc domain, wherein amino acid residue position number is based on EU numbering. In some embodiments, the chemical linker is covalently attached at Lys 248 of an Fc domain of the immunocytokine composition, wherein amino acid residue position number is based on EU numbering. In some embodiments, the chemical linker is covalently attached at Lys 288 of an Fc region of the immunocytokine composition, wherein amino acid residue position number is based on EU numbering. In some embodiments, the chemical linker is covalently attached at Lys 290 of an Fc region of the immunocytokine composition, wherein amino acid residue position number is based on EU numbering. In some embodiments, the chemical linker is covalently attached at Lys 317 of the immunocytokine composition, wherein amino acid residue position number is based on EU numbering.

The chemical linker can be covalently attached to an amino acid residue selected from a subset of amino acid residues. In some embodiments, the subset comprises two three, four, five, six, seven, eight, nine, or ten amino acid residues of an Fc region of the immunocytokine composition. The chemical linker can be covalently attached to one of two lysine residues of an Fc region of the immunocytokine composition.

In some embodiments, the Fc domain will comprise two linkers covalently attached to the Fc region of the immunocytokine composition. In some embodiments, each of the two linkers will be covalently attached to a different heavy chain of the immunocytokine composition. However, in preferred embodiments of immunocytokine compositions described herein, only a single linker is attached to the Fc domain or antigen binding fragment thereof, thereby providing an immunocytokine composition which includes only a single IL-2 polypeptide described herein.

### Linker Points of Attachment to IL-2 Polypeptides

In some embodiments, immunocytokine compositions comprising an IL-2 polypeptide provided herein comprise linkers, including chemical linkers, which link the IL-2 polypeptide to the rest of the immunocytokine composition (e.g., the Fc domain of the immunocytokine composition). When an IL-2 polypeptide is used in an immunocytokine composition, the point of attachment of the linker which links to the IL-2 polypeptide can be any residue as provided herein.

In some embodiments, the linker is attached to an amino acid residue of the IL-2 polypeptide. In some embodiments, the linker is attached to any amino acid residue of the IL-2 polypeptide (e.g., at a position corresponding to any one of positions 1-133 of SEQ ID NO: 701). In some embodiments, the linker is attached at a non-terminal residue (e.g., a residue other than the C-terminal residue or N-terminal residue) of the IL-2 polypeptide (e.g., a residue at position corresponding to any one of positions 2-132 of SEQ ID NO: 701). In some embodiments, the linker is attached at a non-terminal residue of the IL-2 polypeptide, wherein the IL-2 polypeptide has been extended or truncated by one or more amino acids relative to SEQ ID NO: 701 (e.g., the linker is attached to a residue corresponding to residue 2 of SEQ ID NO: 701 and residue 1 of SEQ ID NO: 701 has been deleted). In some embodiments, the linker is attached to the N-terminal residue of the IL-2 polypeptide. In some embodiments, the linker is attached to the N-terminal amine of the IL-2 polypeptide. In some embodiments, the linker is attached to the C-terminal residue of the IL-2 polypeptide. In some embodiments, the linker is attached to the C-terminal carboxyl group of the IL-2 polypeptide.

In some embodiments, the linker is attached to the IL-2 polypeptide at a residue in a region comprising residues 2-132, wherein residue position numbering is based on SEQ ID NO: 701 as a reference sequence. In some embodiments, the linker is attached to the IL-2 polypeptide at a residue in ta region comprising residues 30-75. In some embodiments, the linker is attached to the IL-2 polypeptide at a residue in a region comprising residues 35-55, residues 35-50, residues 35-45, residues 30-50, residues 40-45, residues 60-75, residues 60-70, residues 65-70, or residues 2-5. In some embodiments, the linker is attached to the IL-2 polypeptide at a residue selected from residue 65, 66, 67, 68, 69, and 70. In some embodiments, the linker is attached to the IL-2 polypeptide at a residue selected from residue 40, 41, 42, 43, 44, and 45. In some embodiments, the linker is attached to the IL-2 polypeptide at residue 42 or 45. In some embodiments, the linker is attached to the IL-2 polypeptide at residue 42. In some embodiments, the linker is attached to the IL-2 polypeptide at residue 45.

In some embodiments, the linker is attached to the IL-2 polypeptide at a residue which disrupts binding of the IL-2 polypeptide with the IL-2 receptor alpha subunit (IL-2R). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019028419A1, WO2020056066A1, WO2021140416A2, and WO2021216478A1 each of which is hereby incorporated by reference as if set forth in its entirety. In some embodiments, the linker is covalently attached at a residue selected from residues corresponding to residues 3, 34, 35, 36, 37, 38, 40, 41, 42, 43, 44, 45, 60, 61, 62, 63, 64, 65, 67, 68, 69, 71, 72, 103, 104, 105, and 107 of SEQ ID NO: 701. In some embodiments, the linker is covalently attached at residue 1, 35, 37, 38, 41, 42, 43, 44, 45, 60, 61, 62, 64, 65, 68, 69, 71, 72, 104, 105, or 107 of the IL-2 polypeptide In some embodiments, the linker is covalently attached at residue 5. In some embodiments, the linker is covalently attached at residue 38. In some embodiments, the linker is covalently attached at residue 42. In some embodiments, the linker is covalently attached at residue 45. In some embodiments, the linker is covalently attached at residue 61. In some embodiments, the linker is covalently attached at residue 65. In some embodiments, the linker is covalently attached at residue 68.

In some embodiments, such as those where the IL-2 polypeptide is desired to signal primarily through the IL-2 receptor alpha subunit (e.g., the alpha/beta/gamma receptor complex), the linker can be attached to a residue which disrupts binding of the IL-2 receptor to the IL-2 receptor beta subunit or IL-2 receptor gamma subunit.

In some embodiments, it is desirable that the point of attachment of the linker have no or minimal impact on the ability of the IL-2 polypeptide to interact with any receptor. In such cases, the linker is desirably attached via the N-terminal residue of the IL-2 polypeptide.

In some embodiments, the residue to which the linker is attached is a natural amino acid residue. In some embodiments, the residue to which the linker is covalently attached is selected from cysteine, aspartate, asparagine, glutamate, glutamine, serine, threonine, lysine, and tyrosine. In some embodiments, the residue to which the linker is covalently attached is selected from asparagine, aspartic acid, cysteine, glutamic acid, glutamine, lysine, and tyrosine. In some embodiments, the linker is covalently attached to a cysteine. In some embodiments, the linker is covalently attached to a lysine. In some embodiments, the linker is covalently attached to a glutamine. In some embodiments, the linker is covalently attached to an asparagine. In some embodiments, the residue to which the linker is attached is a tyrosine. In some embodiments, the residue to which the linker is attached is the natural amino acid in that position in SEQ ID NO: 701.

In some embodiments, the linker is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the linker is covalently attached to site-specifically to a natural amino acid.

In some embodiments, the linker is attached at an unnatural amino acid residue. In some embodiments, the unnatural amino acid residue comprises a conjugation handle. In some embodiments, the conjugation handle facilitates the addition of the linker to the modified IL-2 polypeptide. The conjugation handle can be any of the conjugation handles provided herein. In some embodiments, the linker is covalently attached site-specifically to the unnatural amino acid. Non-limiting examples of amino acid residues comprising conjugation handles can be found, for example, in PCT Pub. Nos. WO2015054658A1, WO2014036492A1, and WO2021133839A1 WO2006069246A2, and WO2007079130A2, each of which is incorporated by reference as if set forth in its entirety.

In some embodiments, the linker is covalently attached at residue 42. In some embodiments, the linker is covalently attached at residue F42E, F42D, F42Q, F42K, F42N, or F42Y. In some embodiments, the linker is covalently attached at residue F42Y. In some embodiments, the linker is covalently attached to an unnatural amino acid at residue 42.

In some embodiments, the linker is covalently attached at residue 45. In some embodiments, the linker is covalently attached at residue Y45, Y45E, Y45C, Y45D, Y45Q, Y45K, or Y45N. In some embodiments, the linker is covalently attached at residue Y45. In some embodiments, the linker is covalently attached to an unnatural amino acid at residue 45.

In some embodiments, the linker is covalently attached at residue 65. In some embodiments, the linker is covalently attached at residue P65C, P65D, P65Q, P65E, P65N, P65K, or P65Y. In some embodiments, the linker is covalently attached residue P65C. In some embodiments, the linker is covalently attached to an unnatural amino acid at residue 65.

In some embodiments, the linker is covalently attached at residue 5. In some embodiments, the linker is covalently attached at residue S5C, S5D, S5Q, S5K, S5N, S5K, or S5Y. In some embodiments, the linker is covalently attached residue S5C. In some embodiments, the linker is covalently attached to an unnatural amino acid at residue 5.

In some embodiments, the linker is covalently attached through a modified natural amino acid. In some embodiments, the modified natural amino acid comprises a conjugation handle. In some embodiments, the linker is covalently attached through a modified amino acid α. In some embodiments, the modified amino acid α is an amino-acid-PEG-azide group or an amino-acid-PEG-alkyne group. In some embodiments, the modified amino acid α is a glutamate, aspartate, lysine, cysteine, or tyrosine modified to incorporate an azide, alkyne, or other conjugation handle group linked to the amino acid through a PEG spacer. In some embodiments, the modified amino acid α has a structure selected from:

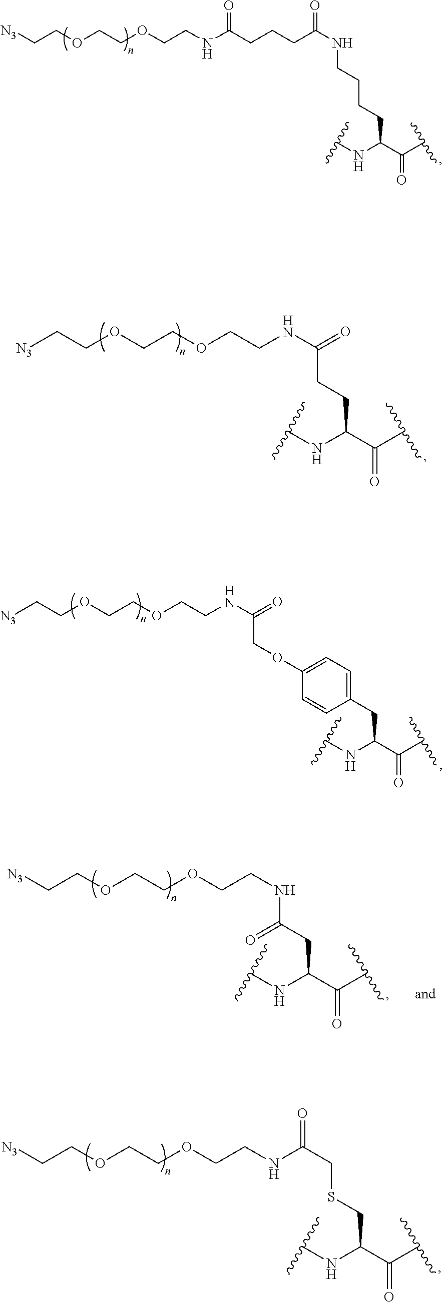

wherein each n is independently an integer from 1-30. In some embodiments, the modified amino acid α has a structure selected from:

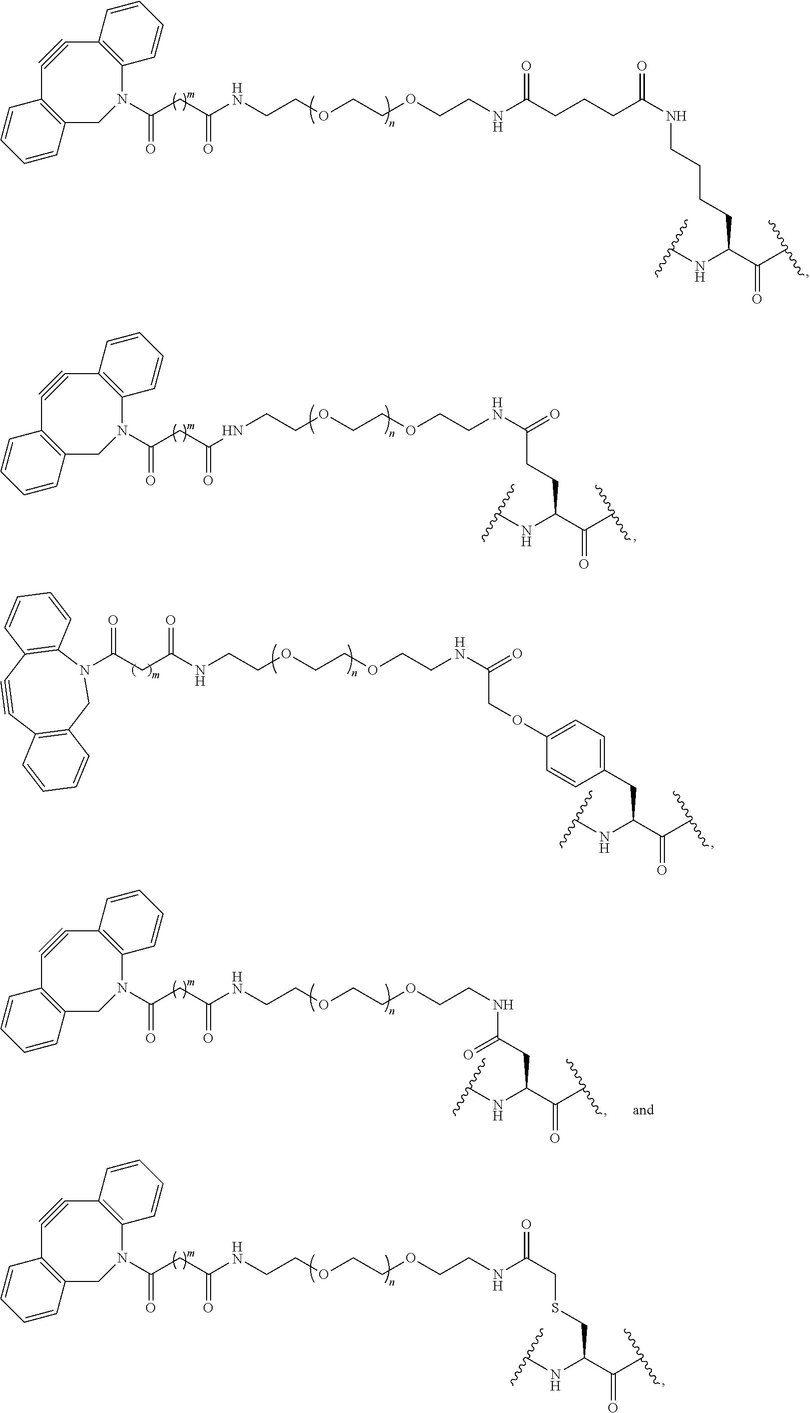

wherein each n is independently an integer from 1-30 and each m is independently an integer from 2-10. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, each m is independently 2, 3, 4, 5, 6, 7, 8, 9, or 10. In some embodiments, m is 3. In some embodiments, m is 4. In some embodiments, m is 5. In some embodiments, m is 6. The modified amino acid α can be incorporated at any point of attachment of the IL-2 polypeptide as provided herein. In some embodiments, the modified amino acid α is located at a position on the IL-2 polypeptide selected from residue 5, residue 42, residue 45, or residue 65. In some embodiments, the modified amino acid a is located at residue 42 of the IL-2 polypeptide. In some embodiments, the modified amino acid a is located at residue 45 of the IL-2 polypeptide.

In some embodiments, the linker is attached to a terminal residue of the IL-2 polypeptide. In some embodiments, the linker is attached to the N-terminal residue of the IL-2 polypeptide. In some embodiments, the linker is attached to the N-terminal amine of the IL-2 polypeptide. In some embodiments, the linker is attached to the N-terminal amine of the IL-2 polypeptide through use of a conjugation handle attached to the N-terminal amine of the IL-2 polypeptide. In some embodiments, the linker is attached to the N-terminal amine of the IL-2 polypeptide through use of a conjugation handle attached to the N-terminal amine of the IL-2 polypeptide through a PEG group. In some embodiments, the conjugation handle comprises an azide or alkyne functionality. In some embodiments, the linker is attached to the N-terminal amine of the IL-2 polypeptide by use of a structure

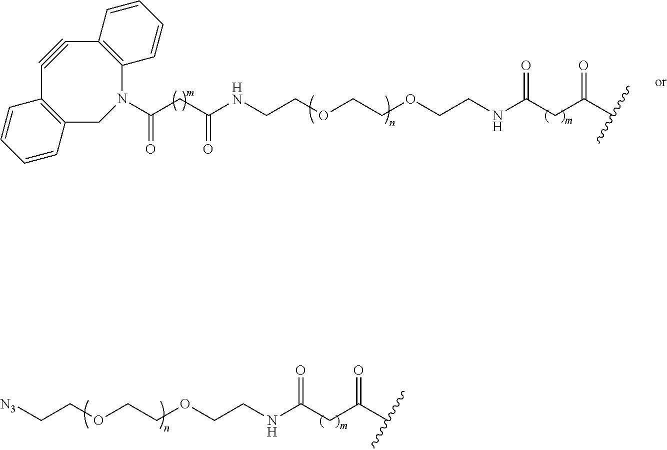

wherein each n is independently an integer from 1-30 and each m is independently an integer from 2-10. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 8. In some embodiments, n is 6. In some embodiments, n is 12. In some embodiments, each m is independently 2, 3, 4, 5, 6, 7, 8, 9, or 10. In some embodiments, m is 3. In some embodiments, m is 4. In some embodiments, m is 5. In some embodiments, m is 6.

In some embodiments, the linker is attached to the C-terminal residue of the IL-2 polypeptide. In some embodiments, the linker is attached to the C-terminal carboxyl group of the Il-2 polypeptide. In some embodiments, the linker is attached to the C-terminal carboxyl of the IL-2 polypeptide through use of a conjugation handle attached to the C-terminal carboxyl of the IL-2 polypeptide. In some embodiments, the linker is attached to the C-terminal carboxyl of the IL-2 polypeptide through use of a conjugation handle attached to C-terminal carboxyl of the IL-2 polypeptide through a PEG group. In some embodiments, the conjugation handle comprises an azide or alkyne functionality.

Where IL-2 polypeptides contain unnatural amino acids or modified natural amino acids (e.g., those provided herein for purposes of conjugation), these amino acids may be incorporated into the IL-2 polypeptides using many techniques known in the art for introduction such modifications. For example, recombinant proteins with unnatural amino acids can be made using methods as described in Patent Cooperation Treaty Publication Nos. WO2016115168, WO2002085923, WO2005019415, and WO2005003294. Alternatively or in combination, unnatural or modified natural amino acids can be incorporated into chemically synthesized proteins during synthesis.

### Methods for Attaching Linkers to Fc Domains

Also provided herein are method of preparing a modified Fc region of an immunocytokine composition, such as for the attachment of a linker, a conjugation handle, the cytokine (e.g., the IL-2 polypeptide), or any combination thereof to the Fc domain to form the immunocytokine composition. A variety of methods for site-specific modification of Fc regions are known in the art.

Modification with an Affinity Peptide Configured to Site-Specifically Attach Linker to the Fc Domain

In some embodiments, an Fc region is modified to incorporate a linker, a conjugation handle, or a combination thereof. In some embodiments, this is accomplished using AJICAP™ technology, or a similar technology known in the art. In some embodiments, the modification is performed by contacting the Fc region with an affinity peptide bearing a payload configured to attach a linker or other group to the Fc region, such as at a specific residue of the Fc region. In some embodiments, the linker is attached using a reactive group which forms a bond with a residue of the Fc region. In some embodiments, the affinity peptide comprises a cleavable linker. The cleavable linker is configured on the affinity peptide such that after the linker or other group is attached to the Fc region, the affinity peptide can be removed, leaving behind only the desired linker or other group attached to the Fc region. The linker or other group can then be used further to add attach additional groups, such as a cytokine or a linker attached to a cytokine, to the Fc region.

Non-limiting examples of such affinity peptides commensurate with AJICAP™ technology can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the immunocytokine composition.

An exemplary affinity peptide with cleavable linker and conjugation handle payload capable of attaching the payload to residue K248 of Fc domain as provided herein can be found as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066.

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). Such alternative affinity peptides include those described in, for example “AJICAP Second Generation: Improved Chemical Site-Specific Conjugation Technology for Antibody-Drug Conjugation Technology for Antibody-Drug Conjugate Production” (Working Paper, Fujii et al., DOI: 10.26434/chemrxiv-2023-9p5p7, chemrxiv.org/engage/chemrxiv/article-details/63d5f7131125965a9e7df8a5 (Accessed 20 Feb. 2023, Version 1 published 30 Jan. 2023)).

The affinity peptide of the disclosure can comprise a cleavable linker. In some embodiments, the cleavable linker of the affinity peptide connects the affinity peptide to the group which is to be attached to the Fc region and is configured such that the peptide can be cleaved after the group comprising the linker or conjugation handle has been attached. In some embodiments, the cleavable linker is a divalent group. In some embodiments, the cleavable linker can comprise a thioester group, an ester group, a sulfane group; a methanimine group; an oxyvinyl group; a thiopropanoate group; an ethane-1,2-diol group; an (imidazole-1-yl)methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linker is:

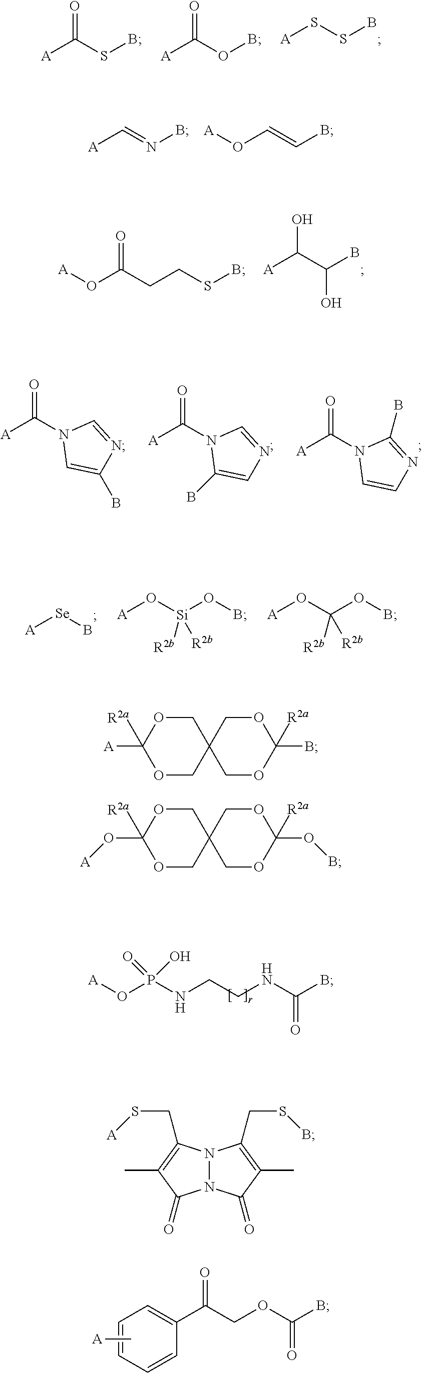

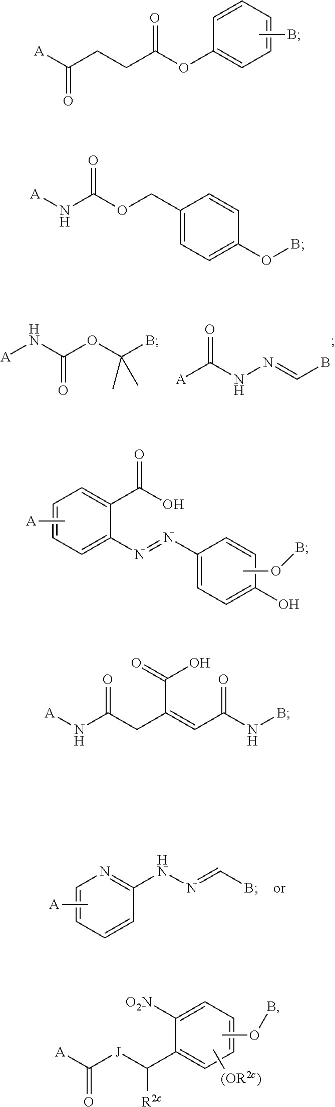

wherein:
one of A or B is a point of attachment the linker and the other of A or B is a point of attachment to the affinity peptide;
each R^{2a} is independently H or optionally substituted alkyl;
each R^{2b} is independently H or optionally substituted alkyl;
R^{2c} is a H or optionally substituted alkyl;
J is a methyl, a N, a S, a Si, or an O atom; and
r is 1, 2, 3, 4, 5, 6, 7, 8, 9, or 10.

The affinity peptide comprises a reactive group which is configured to enable the covalent attachment of the linker/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide). In some embodiments, the reactive group is:

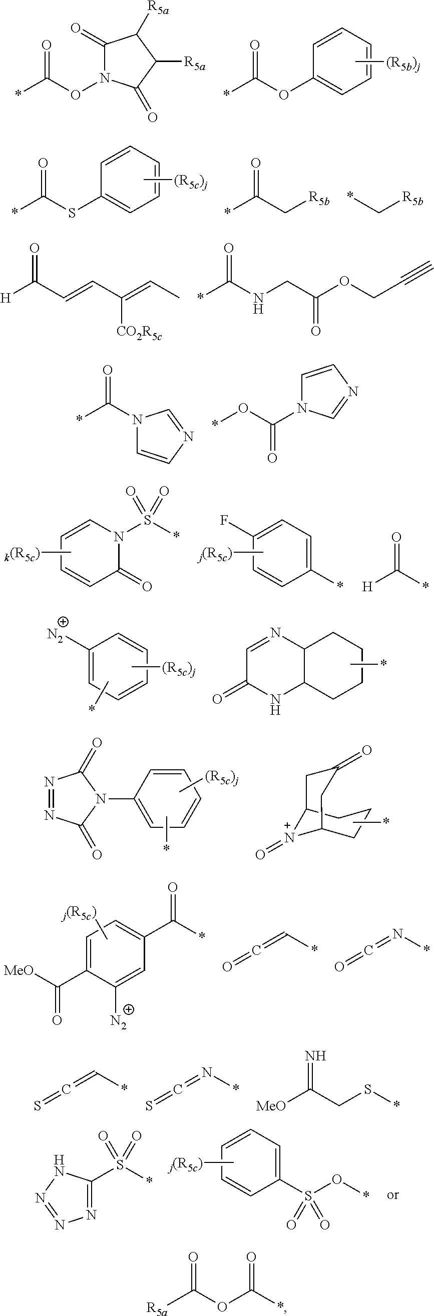

wherein
each R₅ₐ, R_{5b}, and R_{5c} is independently H, halogen, or optionally substituted alkyl;
each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the immunocytokine composition due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linker). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulhydryl group added to the lysine of the Fc region has a structure

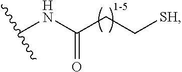

wherein the nitrogen atom shown is the side chain amine of the lysine. Preferably wherein there are 2 or 3 methylenes between the carbonyl and the sulfhydryl group of the structure shown. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B—C, wherein A is the sulfhydryl reactive conjugation handle (e.g., maleimide, α,β-unsaturated carbonyl, a-halogenated carbonyl), B is a linking group, and C is the new conjugation handle (e.g., an alkyne such as DBCO). Specific non-limiting examples of bifunctional linking reagents include

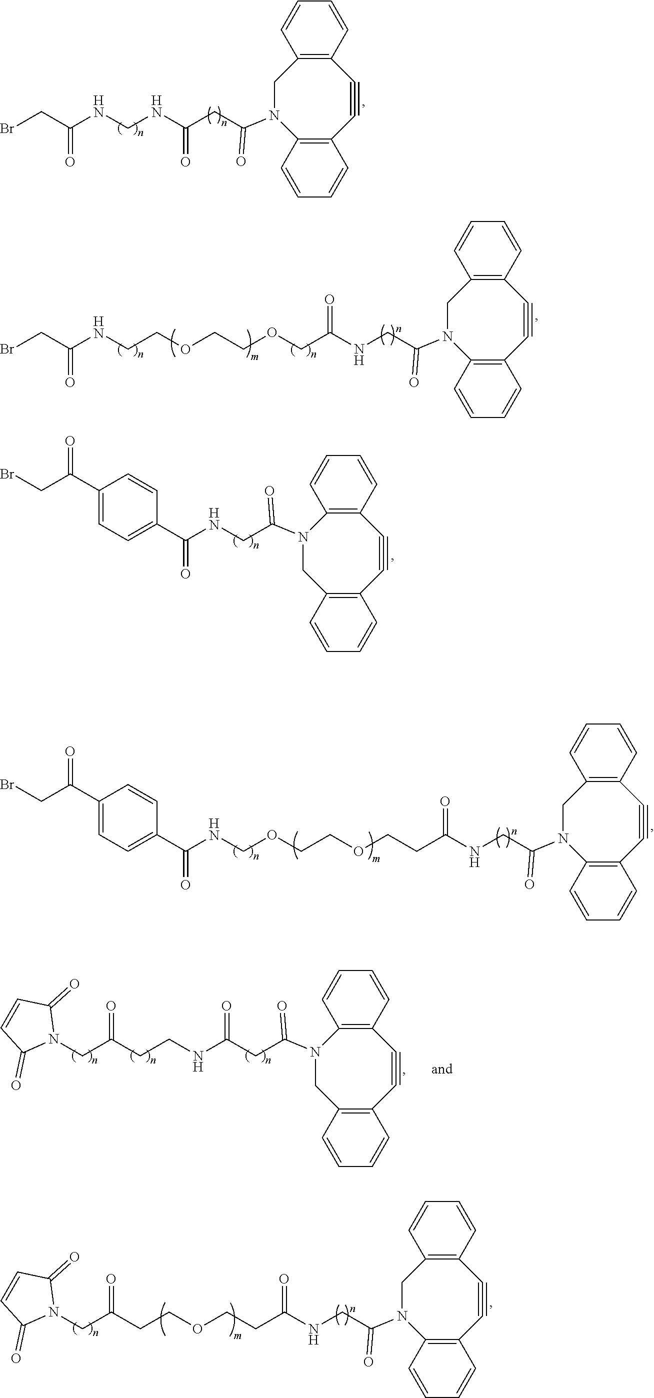

wherein each n is independently an integer from 1-6 and each m is independently an integer from 1-30, and related molecules (e.g., isomers). In such examples, the DBCO group is reacted with an azide group attached to cytokine (e.g. the IL-2 polypeptide (e.g., at any of the residues provided herein, such as C68)).

In some embodiments of immunocytokines described herein, the bifunctional linking reagent used has the structure

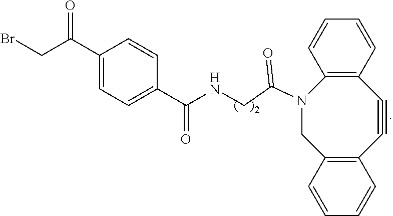

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the cytokine (e.g., the IL-2 polypeptide).

### Alternative Methods of Modifying Fc Region

While the affinity peptide mediated modification of an Fc region provided supra possesses many advantages over other methods which can be used to site-specifically modify the Fc region (e.g., ease of use, ability to rapidly generate many different conjugates, ability to use many “off-the-shelf” commercial antibodies without the need to do time consuming protein engineering, etc.), other methods of performing the modification are also contemplated as being within the scope of the present disclosure.

In some embodiments, a cytokine (e.g., an IL-2 polypeptide) can be conjugated to a suitable Fc Domain utilizing transglutaminase-mediated site-specific antibody-drug conjugate (ADC) strategies, such as those comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications PCT Publication No. WO2020188061, US Patent Publication No. US2019194641, US Patent Publication No. US2021128743, U.S. Pat. Nos. 9,764,038, and 10/434,180, which are incorporated by reference as if set forth herein in their entirety. Such strategies can be employed to either add a suitable conjugation handle to a desired site of the Fc domain (or other part of the immunocytokine composition), or can alternatively be used to directly conjugate the cytokine (e.g., the IL-2 polypeptide) to the Fc domain, though it is preferable that such a strategy is used to add a conjugation handle, then the cytokine (e.g. IL-2 polypeptide) is later reacted with the conjugation handle to form the immunocytokine.

In some embodiments, a cytokine (e.g. IL-2 polypeptide) can be conjugated to a suitable Fc domain utilizing a transpeptide-mediated strategy for either direct attachment of the cytokine (e.g., IL-2 polypeptide) to the immunocytokine composition or via addition of a suitable conjugation handle to the Fc domain. For example, a sortase based system (such as those described in, for example, U.S. Pat. Nos. 10,081,684, 10,864,277, 11,421,022, and/or 9,862,779) can be used, either by addition of a sortase tag to the C-terminus of the Fc domain or through a suitable linker armed with a sortase tag, to add a linker with a conjugation handle to the Fc domain, followed by a subsequent conjugation of the cytokine to the immunocytokine composition using the conjugation handle.

In another aspect, the disclosure provides methods of generating immunocytokines using an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the immunocytokine composition comprises a full length antibody heavy chain and an antibody light chain, wherein the acyl donor glutamine-containing tag is located at the carboxyl terminus of a heavy chain, a light chain, or both the heavy chain and the light chain.

In some embodiments, the immunocytokine composition comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, provided herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, provided herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence GGLLQGPP (SEQ ID NO: 299), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, provided herein is a method for conjugating a moiety of interest (Z) to an Fc domain, comprising the steps of: (a) providing an Fc domain having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said Fc domain with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an Fc domain comprising an acceptor amino acid residue linked (covalently) to a reactive group (R) via the linking reagent. Optionally, said acceptor residue of the Fc domain or Fc domain fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain optionally further-within the CH2 domain Optionally, the Fc domain is free of heavy chain N297-linked glycosylation. Optionally, the acceptor glutamine is at position 295 and the residue at the +2 position is the residue at position 297 (EU index numbering) of an Fc domain.

In one aspect, provided herein is a method for conjugating a moiety of interest (Z) to an Fc domain, comprising the steps of: (a) providing an Fc domain having at least one acceptor glutamine residue; and (b) reacting said Fc domain with a linker comprising a primary amine (a lysine-based linker) comprising a reactive group (R), preferably a protected reactive group, in the presence of a transglutaminase (TGase), under conditions sufficient to obtain an Fc domain comprising an acceptor glutamine linked (covalently) to a reactive group (R) via said linker. Optionally, said acceptor glutamine residue of the Fc domain is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an Fc domain, optionally further-within the CH2 domain Optionally, the Fc domain is free of heavy chain N297-linked glycosylation. Optionally, the acceptor glutamine is at position 295 and the residue at the +2 position is the residue at position 297 (EU index numbering) of an Fc domain. The Fc domain comprising an acceptor residue or acceptor glutamine residue linked to a reactive group (R) via a linker comprising a primary amine (a lysine-based linker) can thereafter be reacted with a reaction partner comprising a moiety of interest (Z) to generate an Fc domain comprising an acceptor residue or acceptor glutamine residue linked to a moiety of interest (Z) via the linker. Thus, in one embodiment, the method further comprises a step (c): reacting (i) an Fc domain of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an Fc domain comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the Fc domain. In one embodiment, the Fc domain comprises two acceptor glutamines and the compound comprising a moiety of interest (Z) and a reactive group (R′) is provided at 10 or less molar equivalents to the Fc domain. In one embodiment, the Fc domain comprises two acceptor glutamines and the compound comprising a moiety of interest (Z) and a reactive group (R′) is provided at 5 or less molar equivalents to the Fc domain. In one embodiment, the Fc domain comprises four acceptor glutamines and the compound comprising a moiety of interest (Z) and a reactive group (R′) is provided at 20 or less molar equivalents to the Fc domain. In one embodiment, the Fc domain comprises four acceptor glutamines and the compound comprising a moiety of interest (Z) and a reactive group (R′) is provided at 10 or less molar equivalents to the Fc domain. In one embodiment, steps (b) and/or (c) are carried out in aqueous conditions. Optionally, step (c) comprises: immobilizing a sample of an Fc domain comprising a functionalized acceptor glutamine residue of Formula II on a solid support to provide a sample comprising immobilized antibodies, reacting the sample comprising immobilized antibodies, optionally recovering any unreacted compound and re-introducing such recovered compound to the solid support for reaction with immobilized antibodies, and eluting the Fc domain conjugates to provide an Fc domain composition comprising a Z moiety.

In an alternative embodiment, an amino acid residue comprising a conjugation handle can be incorporated into the Fc domain (e.g., during expression of the Fc domain) at a desired location (e.g., any of the locations provided herein). In some embodiments, the amino acid residue comprising the conjugation handle is an unnatural amino acid.

### Conjugation Handle Chemistry

In some embodiments, the appropriately modified Fc region of the immunocytokine composition will comprise a conjugation handle which is used to conjugate the immunocytokine composition to a cytokine (e.g. IL-2 polypeptide) to produce an immunocytokine composition provided herein.

Any suitable reactive group capable of reacting with a complementary reactive group attached to the cytokine can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cyclooctenes, potassium acyl trifluoroborate (KAT) ligation or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne, azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, potassium acyl trifluoroborate, hydroxylamine (e.g., 0-substituted hydroxylamine) and hydrazide. In some embodiments, the cytokine comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research, volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

### Linker Structure

In some embodiments, the linker used to attach the Fc domain and the cytokine (e.g., IL-2 polypeptide) comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein). In some embodiments, the linker is a peptide linker. In some embodiments, the linker is a non-peptide linker (e.g., does not consist of amino acid residues).

### Chemical Linkers

In some embodiments, the linker is a chemical linker. Chemical linkers are generally used in the context of chemically conjugated multifunctional immunocytokines (e.g., immunocytokines in which the cytokine is conjugated to a scaffold comprising the binding domains specific for PD-1 and VEGFA). In some embodiments, the chemical linker comprises at least one portion which is not comprised of amino acid residues. In some embodiments, the linker comprises a polymer. In some embodiments, the linker comprises a water soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker is branched and comprises a linear portion of a chain of at least 10, 20, 50, 100, 500, 1000, 2000, 3000, or 5000 atoms. In some embodiments, the linker comprises a linear portion of at from 1 to 1000 atoms, 1 to 900 atoms, 1 to 800 atoms, 1 to 500 atoms, 1 to 400 atoms, 1 to 300 atoms, 1 to 200 atoms, 1 to 100 atoms, 1 to 50 atoms, 10 to 1000 atoms, 10 to 900 atoms, 10 to 800 atoms, 10 to 500 atoms, 10 to 400 atoms, 10 to 300 atoms, 10 to 200 atoms, 10 to 100 atoms, 10 to 50 atoms, 25 to 1000 atoms, 25 to 900 atoms, 25 to 800 atoms, 25 to 500 atoms, 25 to 400 atoms, 25 to 300 atoms, 25 to 200 atoms, 25 to 100 atoms, 25 to 50 atoms, 50 to 1000 atoms, 50 to 900 atoms, 50 to 800 atoms, 50 to 500 atoms, 50 to 400 atoms, 50 to 300 atoms, 50 to 200 atoms, or 50 to 100 atoms. In some embodiments, the linker has a linear length of from about 10 angstroms to about 200 angstroms. In some embodiments, the linker has a linear length of from about 10 to 500, 10 to 200, 10 to 150, 10 to 125, 10 to 100, 10 to 75, 10 to 50, 25 to 200, 25 to 150, 25 to 125, 25 to 100, 25 to 75, 25 to 50, 50 to 200, 50 to 150, 50 to 100, or 50 to 75 angstroms.

In some embodiments, the linker has a molecular weight of about 200 Daltons to about 2000 Daltons. In some embodiments, the linker has a molecular weight of about 200 Daltons to about 5000 Daltons. In some embodiments, the linker has a molecular weight of 200 Daltons to 100,000 Daltons. In some embodiments, the linker has a molecular weight of at least about 500 Daltons, at least about 1,000 Daltons, at least about 5,000 Daltons, at least about 10,000 Daltons, at least about 15,000 Daltons, at least about 20,000 Daltons, at least about 25,000 Daltons, or at least about 30,000 Daltons. In some embodiments, the linker as a molecular weight of at most about 100,000 Daltons, at most about 50,000 Daltons, at most about 40,000 Daltons, at most about 30,000 Daltons, at most about 25,000 Daltons, at most about 20,000 Daltons at most about 15,000 Daltons, at most about 10,000 Daltons, or at most about 5,000 Daltons.

In some embodiments, the linker comprises a reaction product of one or more pairs of conjugation handles and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other is.

In some embodiments, the linker comprises a structure of Formula (X)

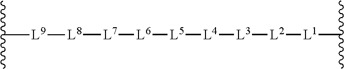

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O— CH(CH₃)—CH₂)_{qd}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

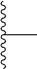

is a point of attachment to the Fc domain or the cytokine (e.g., the IL-2 polypeptide).

In some embodiments, the linker consists of a plurality of structures of Formula (X) to form the linkage between the Fc domain and the cytokine (e.g., the IL-2 polypeptide) (e.g., 1, 2, 3, 4, 5, 6, 7, 8, 9, 10, or more structures of Formula (X) appended from end to end, where only the terminal

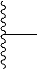

denote points of attachment to the Fc domain or the cytokine (e.g., IL-2 polypeptide)).

In some embodiments, the polymer comprises a linker comprising a structure of Formula (X′)

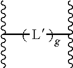

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O), —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O— CH(CH₃)—CH₂)_{qd}—, a reaction product of a conjugation handle and a complementary conjugation handle, or absent; (C₁-C₆ alkylene);
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₅ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
g is an integer from 1-100,
wherein each

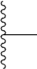

is a point of attachment to the cytokine (e.g., IL-2 polypeptide) or the Fc domain.

In some embodiments, the linker of Formula (X) or Formula (X′) comprises the structure:

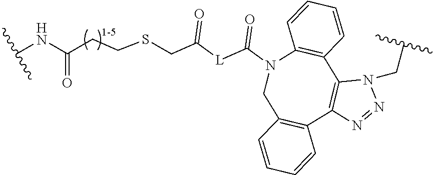

wherein

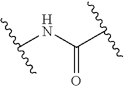 [SMILES: *NC(*)=O]

is the attachment to a lysine residue of the Fc domain;
L is a linking group; and

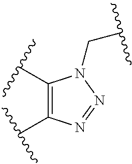 [SMILES: *Cn1nnc(*)c1*]

is a point of attachment to a linking group which connects to point of attachment on the cytokine (e.g. IL-2 polypeptide),
or a regioisomer thereof.

In some embodiments, L has a structure

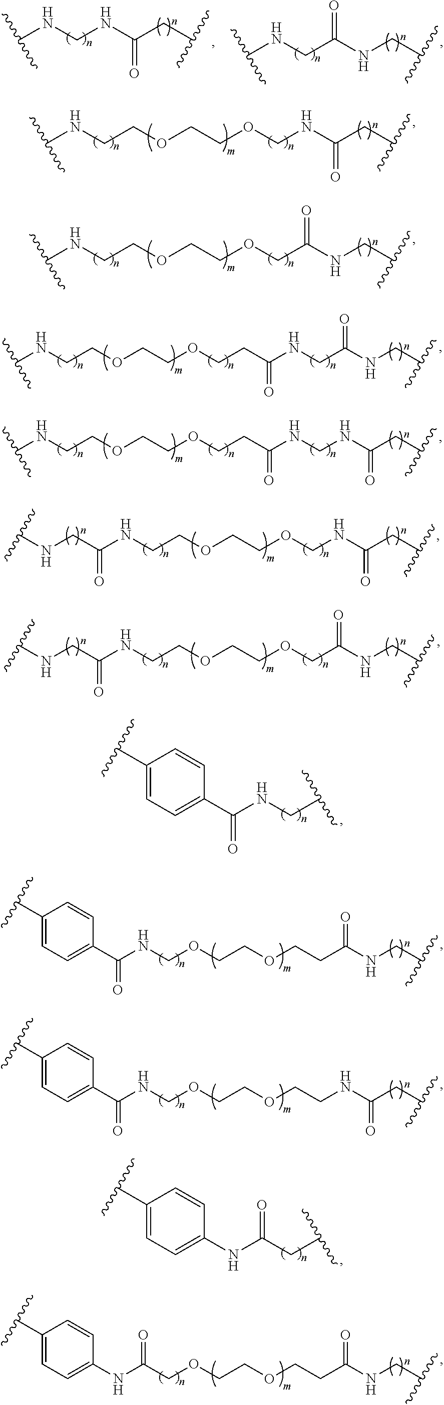

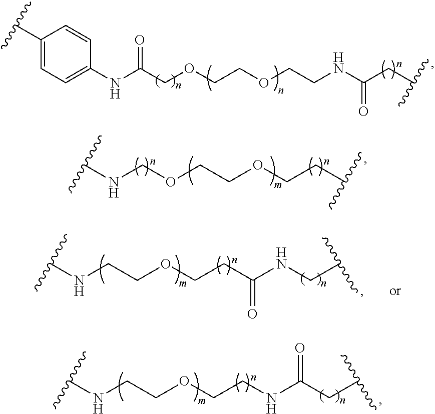

wherein each n is independently an integer from 1-6 and each m is an integer from 1-30. In some embodiments, each m is independently 2 or 3. In some embodiments, each m is an integer from 1-24, from 1-18, from 1-12, or from 1-6.

In some embodiments, the linker of Formula (X) or of Formula (X′) comprises the structure:

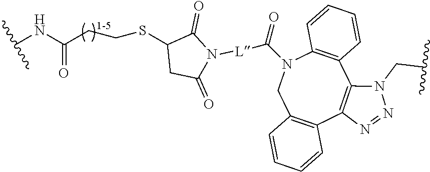

wherein

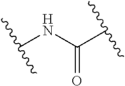 [SMILES: *NC(*)=O]

is the first point of attachment to a lysine residue of the Fc domain of the immunocytokine composition;
L″ is a linking group; and

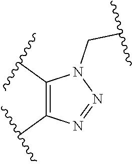 [SMILES: *Cn1nnc(*)c1*]

is a point of attachment to a linking group which connects to the cytokine (e.g. the IL-2 polypeptide),
or a regioisomer thereof.

In some embodiments, L″ has a structure

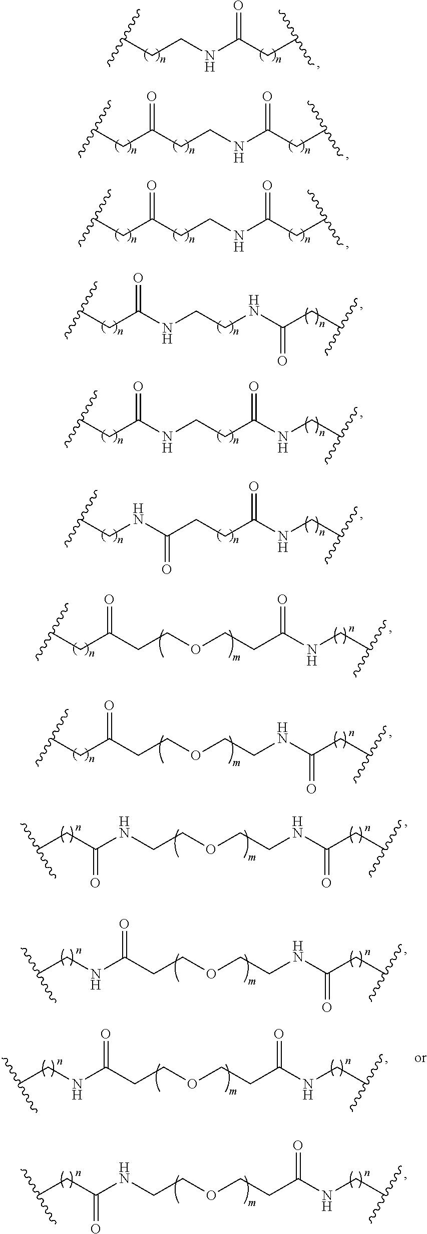

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

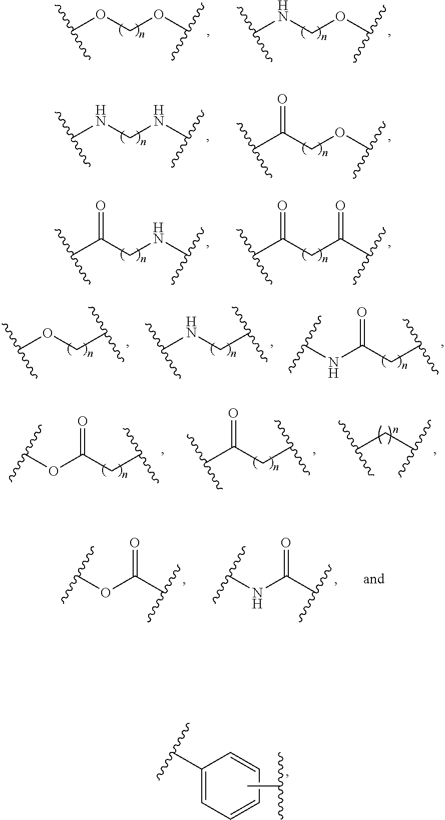

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

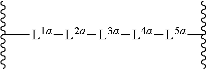

wherein each of L^{1a}, L^{2a}, L^{3a}, L^{4a}, L^{5a}, is independently —O—, —NR^{La}—, —(C₁-C₆ alkylene)NR^{La}—, —NR^{La}(C₁-C₆ alkylene)-, —N(R^{L})₂⁺, —(C₁-C₆ alkylene)N(R^{La})₂⁺(C₁-C₆ alkylene)-, —N(R^{L})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O)NR^{La}(C₁-C₆ alkylene)-, —(C₁-C₆ alkylene)C(═O)NR^{La}—, —NR^{La}C(═O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}═N—, —N═CR^{La}, —NR^{La}S(═O)₂—, —S(═O)₂NR^{La}—, —C(═O)NR'S(═O)₂—, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}—, —(O—CH(CH₃)—CH₂)_{qh}—, a reaction product of a conjugation handle and a complementary conjugation handle, or absent; (C₁-C₆ alkylene)
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the linking group which connects to the point of attachment of the cytokine, (e.g., the IL-2 polypeptide) comprises poly(ethylene glycol). In some embodiments, the linking group comprises about 2 to about 30 poly(ethylene glycol) units. In some embodiments, the linking group which connects to the point of attachment of the cytokine (e.g. the IL-2 polypeptide) is a functionality attached to a cytokine provided herein which comprises an azide (e.g., the triazole is the reaction product of the azide).

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle comprise a structure of

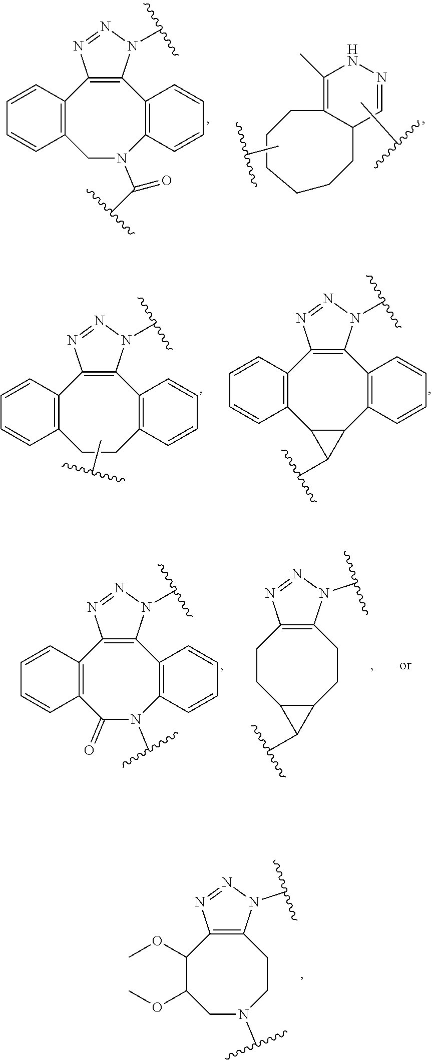

or a regioisomer or derivative thereof.

In some embodiments, the linker has a structure

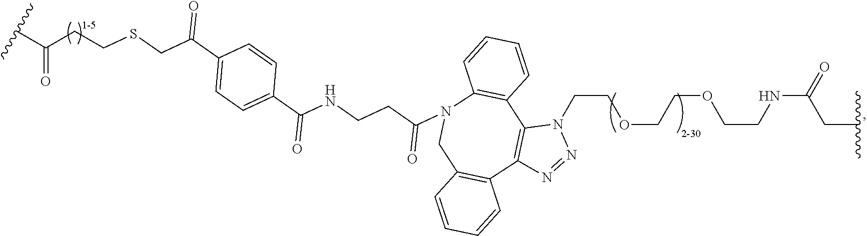

wherein the carbonyl on the left side of the molecule is attached to an Fc domain lysine (e.g., K248) of the immunocytokine composition and the right side of the molecule is attached to the cytokine (e.g., F42Y, Y45, or the N-terminus of an IL-2 polypeptide), or is part of a linker linked to the cytokine (e.g., the right side of the molecule is linked to an additional carbonyl which forms an amid bond to the N-terminus of an IL-2 polypeptide). In some embodiments of the immunocytokines provided herein, the linker has the structure

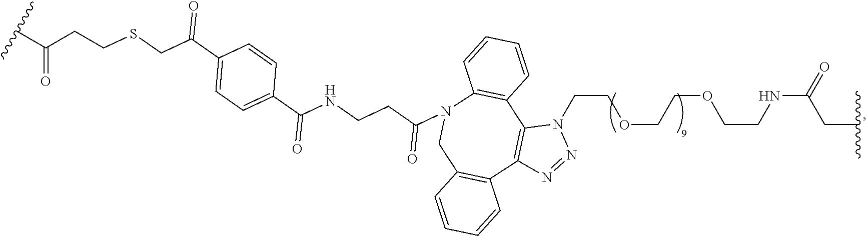

wherein the carbonyl on the left side of the molecule is attached to an Fc domain lysine (e.g., K248) and the right side of the molecule is attached to residue F42Y of an IL-2 polypeptide as provided herein (or to another suitable residue). In some embodiments of the immunocytokines provided herein, the linker has the structure

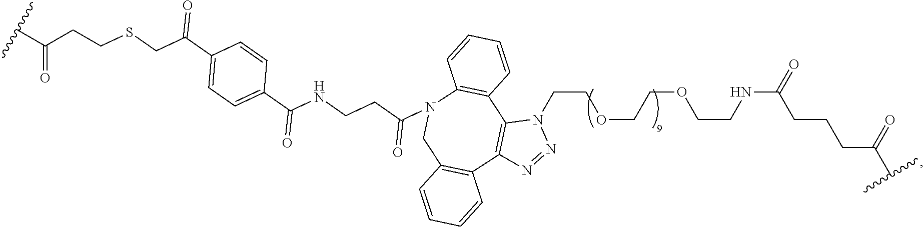

wherein the carbonyl on the left side of the molecule is attached to an Fc domain lysine (e.g., K248) and the right side of the molecule is attached to the N-terminal amine of an IL-2 polypeptide as provided herein (or to another suitable residue).

### Peptide Linkers

In some embodiments, the Fc domain is linked to the other components of the immunocytokine composition (e.g., the binding domains or the cytokine, such as the IL-2 polypeptide). In some embodiments, the Fc domain is linked to the cytokine (e.g., the IL-2 polypeptide) as a fusion protein. In some embodiments, the Fc domain is linked to both binding domains as a fusion protein. In such instances, the linker (if present) comprises one or more peptide bonds between the Fc domain and the other group (e.g., the binding domain or the cytokine). In some embodiments, the linker between the Fc domain and the binding domain or the cytokine is a bond. In some embodiments, the linker between the Fc domain and the binding domain or cytokine is a linking peptide. Non-limiting examples of linking peptides include, but are not limited to (GS)ₙ (SEQ ID NO: 23), (GGS)ₙ (SEQ ID NO: 24), (GGGS)ₙ (SEQ ID NO: 25), (GGSG)ₙ (SEQ ID NO: 26), or (GGSGG)ₙ (SEQ ID NO: 27), (GGGGS)ₙ (SEQ ID NO: 28), wherein n is 1, 2, 3, 4, 5, 6, 7, 8, 9, or 10. For example, a linking peptide can be GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, a binding domain or cytokine is fused to the C-terminal end of the Fc domain (optionally through a linking peptide). In some embodiments, the binding domain or cytokine is fused to the N-terminal end of the Fc domain (optionally through a linking peptide). In embodiments wherein multiple portions of the immunocytokine composition are fused to the Fc domain, each peptide linker is independently selected and can be the same or different.

In embodiments in which a binding domain or the cytokine is fused to the N-terminus of the Fc domain, the linking peptide desirably is or comprises a hinge region of an antibody. In some embodiments (e.g., those in which the binding domain is a Fab), the linking peptide is a hinge region. For example, when the Fc domain is derived from an IgG1, an IgG1 hinge region or variant thereof can desirably be used to link the binding domain to the Fc domain (e.g., a hinge region having a sequence EPKSCDKTHTCPPCPAPELLGGP (SEQ ID NO: 267) or a variant thereof (e.g., having a sequence at least 80%, 85%, 90%, 95%, 96%, 97%, 98%, or 99% identity thereto), such as a so-called “LALA” variant which comprises L234A and L235A substitutions (EU numbering) (i.e., having a sequence EPKSCDKTHTCPPCPAPEAAGGP (SEQ ID NO: 268)). In some embodiments, the hinge region comprises a sequence having at least 80%, 85%, 90%, 95%, or 100% identity to the sequence EPKSCDKTHTCPPCP (SEQ ID NO: 269). Similarly, when the Fc domain is derived from an IgG4, an IgG4 hinge region or variant thereof can serve as the linker between the binding domain (e.g., the Fab or scFv) and the Fc domain (e.g., an IgG4 hinge region of the sequence ESKYGPPCPSCPAPEFLGGP (SEQ ID NO: 270) or a variant thereof (e.g., having a sequence at least 80%, 85%, 90%, 95%, 96%, 97%, 98%, or 99% identity thereto)). In some embodiments, the hinge region comprises a sequence having at least 80%, 85%, 90%, 95%, or 100% identity to the sequence ESKYGPPCPSCP (SEQ ID NO: 271). In some embodiments, the linker peptide comprises an antibody hinge region, or an amino acid sequence having 1, 2, 3, 4, or 5 substitutions thereto (e.g., conservative substitutions). In some embodiments, a hinge region acts as the linker between a Fab binding domain as described herein and an Fc domain.

In some embodiments, the linking peptide comprises a portion of a hinge region. In some embodiments, the linking peptide comprises a hinge region portion having the amino acid sequence DKTHTCPPCPAPELLGGP (SEQ ID NO: 265), or a sequence at least 80%, 85%, 90%, 95%, 96%, 97%, 98%, or 99% identity thereto. In some embodiments, the linking peptide comprises a hinge region portion having the amino acid sequence DKTHTCPPCPAPEAAGGP (SEQ ID NO: 266), or a sequence at least 80%, 85%, 90%, 95%, 96%, 97%, 98%, or 99% identity thereto.

In some embodiments, the linking peptide comprises a GS linker (e.g., any one of SEQ ID NOs: 21-30) and at least a portion of a hinge region (e.g., any of the hinge regions described in the preceding paragraph). In some embodiments, a linking peptide comprises a GS linker (e.g., any of one of SEQ ID NOs: 21-30) and a hinge region peptide having an amino acid sequence DKTHTCPPCPAPELLGGP (SEQ ID NO: 265), or a sequence at least 80%, 85%, 90%, 95%, 96%, 97%, 98%, or 99% identity thereto. In some embodiments, a linking peptide comprises a GS linker (e.g., any of one of SEQ ID NOs: 21-30) and a hinge region peptide having an amino acid sequence DKTHTCPPCPAPEAAGGP (SEQ ID NO: 266), or a sequence at least 80%, 85%, 90%, 95%, 96%, 97%, 98%, or 99% identity thereto. In some embodiments, such linking peptides comprising a GS linker and a hinge region peptide are used to link the peptide to the N-terminus of the Fc region (e.g., to the N-terminus of the CH₂ domain).

In some embodiments, the cytokine (e.g., the IL-2 polypeptide) is fused to N-terminus of the Fc region via a linking peptide comprising a GS linker (e.g., any one of SEQ ID NOs: 21-30) and a least a portion of a hinge region. In some embodiments, the cytokine (e.g., the IL-2 polypeptide) is fused to N-terminus of the Fc region via a linking peptide comprising a GS linker (e.g., any one of SEQ ID NOs: 21-30) and a hinge region peptide having an amino acid sequence of SEQ ID NO: 265 or 266, or a sequence at least 80%, 85%, 90%, 95%, 96%, 97%, 98%, or 99% identity thereto. In some embodiments, the cytokine (e.g., the IL-2 polypeptide) is fused to N-terminus of the Fc region via a linking peptide comprising a GS linker (e.g., any one of SEQ ID NOs: 21-30) and a hinge region peptide having an amino acid sequence of SEQ ID NO: 265 or 266. In some embodiments, the cytokine (e.g., the IL-2 polypeptide) is fused to N-terminus of the Fc region via a linking peptide comprising a GS linker of SEQ ID NO: 22 and a hinge region peptide having an amino acid sequence of SEQ ID NO: 265 or 266. In some embodiments, the cytokine (e.g., the IL-2 polypeptide) is fused to N-terminus of the Fc region via a linking peptide comprising a GS linker of SEQ ID NO: 22 and a hinge region peptide having an amino acid sequence of SEQ ID NO: 265. In some embodiments, the cytokine (e.g., the IL-2 polypeptide) is fused to N-terminus of the Fc region via a linking peptide comprising a GS linker of SEQ ID NO: 22 and a hinge region peptide having an amino acid sequence of SEQ ID NO: 266.

### Exemplary Fusion Immunocytokine Compositions Having an Anti-PD-1 Fab and a C-Terminal Fused IL-2

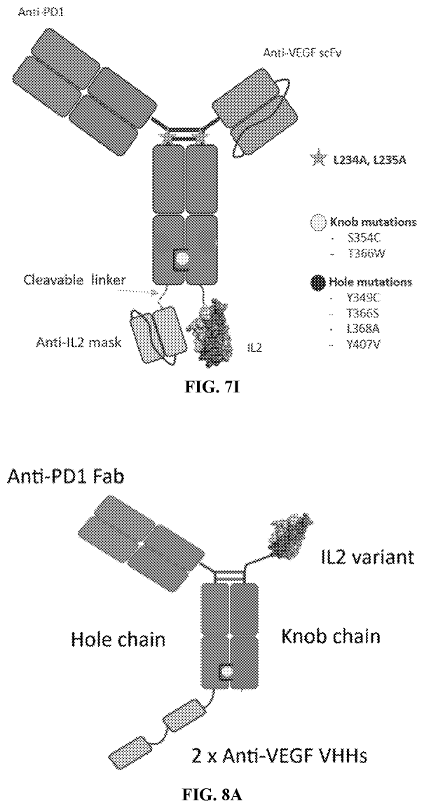

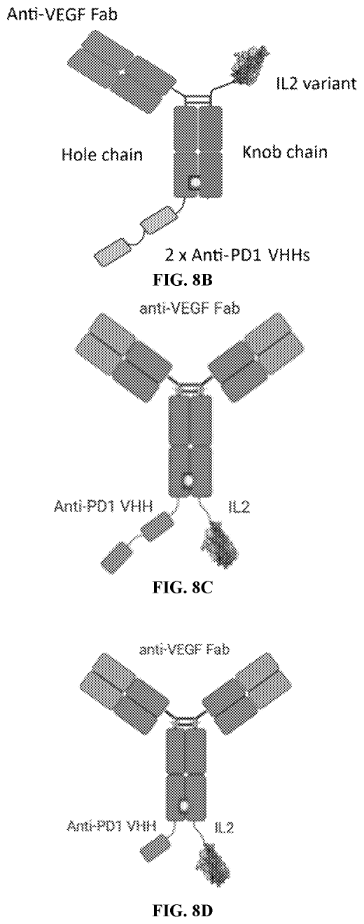

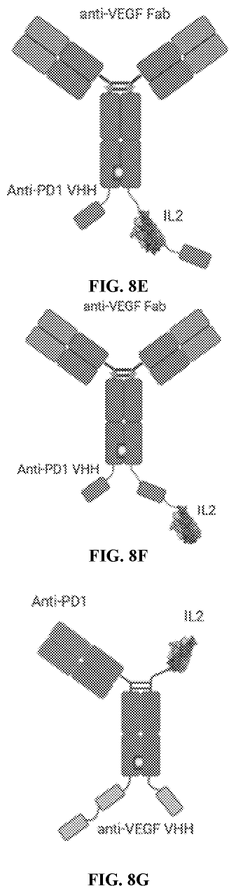

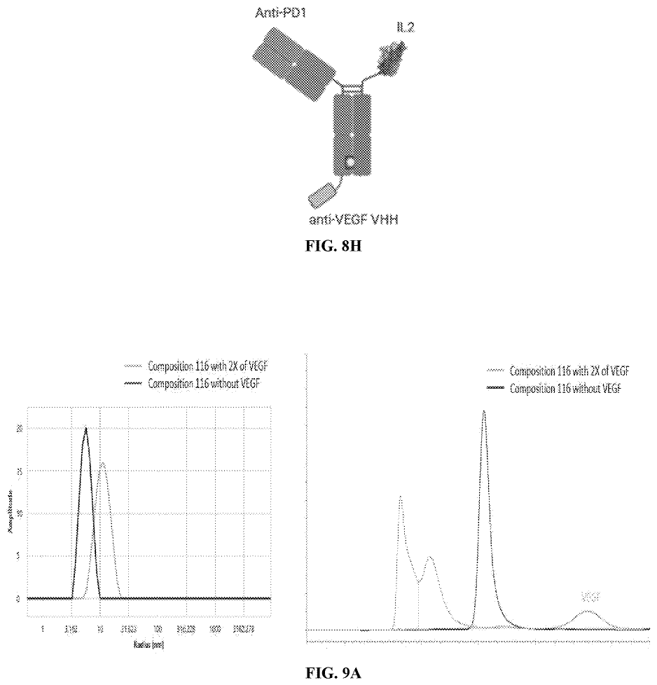

In the following section are exemplary formats of immunocytokine compositions described herein prepared as fusion proteins which comprise a Fab domain targeting PD-1 (e.g., any of the Fab domains described herein, such as a Fab domain of an anti-PD-1 antibody such as Pembrolizumab, Nivolumab, LZM-009, or another suitable anti-PD-1 antibody), one or more binding domains targeting VEGF (e.g., any of the VHH domains targeting VEGF described herein) and an IL-2 polypeptide (e.g., any of the IL-2 polypeptides described herein) fused via its C-terminus to an Fc domain opposite the Fc domain harboring the anti-PD-1 Fab (e.g., as depicted in FIG. 6A, FIG. 6B, FIG. 8A, FIG. 8G, and FIG. 8H. In some embodiments described in this section, the formats described are those depicted in FIG. 6B and FIG. 8A which contain two anti-VEGF VHH domains, positioned either in series attached to the C-terminus of the Fc domain harboring the anti-PD-1 Fab via an optional peptide linker (as in FIG. 8) or with one anti-VEGF VHH domain attached to the C-terminus of the Fc domain harboring the anti-PD-1 Fab via an optional peptide linker and with the second anti-VEGF VHH domain attached to the C-terminus of the Fc domain harboring the IL-2 polypeptide via another optional peptide linker (as in FIG. 6B). In the version depicted in FIG. 8 with the two anti-VEGF VHH domains positioned in series, each anti-VEGF VHH domain can be separated by a peptide linker. Analogous formats and the other formats described herein are contemplated as also within the scope of the instant disclosure. Throughout this section, the “first polypeptide chain” refers to a polypeptide comprising the VL of the anti-PD-1 Fab, the “second polypeptide chain” refers to a polypeptide comprising the VH of the anti-PD-1 Fab, and the “third polypeptide chain” refers to the polypeptide comprising the IL-2 polypeptide. Analogous constructs in which the VEGFA and PD-1 binding domains are swapped (e.g., any of the constructs described in this section, but each PD-1 binding domain is swapped for an analogous VEGFA binding domain and vice versa) are also contemplated herein (e.g., as depicted in FIG. 6C and FIG. 8B).

In some embodiments herein is an immunocytokine composition in which the first binding domain is specific for PD-1 and comprises a Fab having a VH and a VL, and wherein the composition comprises: a) a first polypeptide chain comprising the VL of the first binding domain; b) a second polypeptide chain comprising the VH of the first binding domain; and c) a third polypeptide chain comprising the IL-2 polypeptide.

In some embodiments, the first binding domain Fab comprises a VH having a VH CD1, VH CDR2, and VH CDR3 and a VL having a VL CDR1, CDR2, and CDR3 of any one of the anti-PD-1 antibodies described herein (e.g., any of the antibodies described in Table 1A). In some embodiments, the first binding domain Fab comprises:
a VH CDR1, VH CDR2, and VH CDR3 of SEQ ID NOs: 80, 81, and 82, respectively and a VL CDR1, VL CDR2, and VL CDR3 of SEQ ID NOs: 83, 84, and 85, respectively;
a VH CDR1, VH CDR2, and VH CDR3 of SEQ ID NOs: 86, 87, and 88, respectively and a VL CDR1, VL CDR2, and VL CDR3 of SEQ ID NOs: 89, 90, and 91, respectively; or
a VH CDR1, VH CDR2, and VH CDR3 of SEQ ID NOs: 113, 114, and 115, respectively and a VL CDR1, VL CDR2, and VL CDR3 of SEQ ID NOs: 83, 117, and 118, respectively.

In some embodiments, the first binding domain Fab comprises a VH and a VL of any one of the anti-PD-1 antibodies described herein (e.g., any of the antibodies described in Table 1A), or a sequence having at least 80%, 85%, 90%, 95%, 96%, 97%, 98%, or 99% identity thereto (preferably wherein the CDRs are retained). In some embodiments, the first binding domain Fab comprises: a) a VH comprising an amino acid sequence amino acid sequence having at least 80%, 85%, 90%, 95%, 96%, 97%, 98%, 99%, or 100% identity to SEQ ID NO: 48 and a VL comprising an amino acid sequence having at least 80%, 85%, 90%, 95%, 96%, 97%, 98%, 99%, or 100% identity to SEQ ID NO: 49; b) a VH comprising an amino acid sequence amino acid sequence having at least 80%, 85%, 90%, 95%, 96%, 97%, 98%, 99%, or 100% identity to SEQ ID NO: 50 and a VL comprising an amino acid sequence having at least 80%, 85%, 90%, 95%, 96%, 97%, 98%, 99%, or 100% identity to SEQ ID NO: 51; or c) a VH comprising an amino acid sequence amino acid sequence having at least 80%, 85%, 90%, 95%, 96%, 97%, 98%, 99%, or 100% identity to SEQ ID NO: 76 and a VL comprising an amino acid sequence having at least 80%, 85%, 90%, 95%, 96%, 97%, 98%, 99%, or 100% identity to SEQ ID NO: 77.

In some embodiments, the first polypeptide chain comprises, in N- to C-terminal direction, the VL and a light chain constant region. In some embodiments, the light chain constant region comprises an amino acid having at least 80%, 85%, 90%, 95%, 96%, 97%, 98%, 99%, or 100% sequence identity to SEQ ID NO: 276, 277, or 278.

In some embodiments, the first polypeptide chain comprises the sequence set forth in SEQ ID NO: 47 or SEQ ID NO: 51 or 57 fused to the sequence of SEQ ID NO: 276.

In some embodiments, the second polypeptide chain comprises, in an N-terminal to C-terminal direction, the VH of the Fab of the first binding domain and an antibody constant region. In some embodiments, the antibody constant region is an IgG1 or an IgG4. In some embodiments, the IgG1 or IgG4 comprises an amino acid sequence having at least 90%, 95%, 96%, 97%, 98%, 99%, or 100% identity to a wild type IgG1 or IgG4 sequence.

In some embodiments the antibody constant region comprises, in an N-terminal to C-terminal direction, a CH1 domain, a hinge region, a CH2 domain, and a CH3 domain. In some embodiments, the CH1 domain comprises an amino acid sequence having at least 80%, 85%, 90%, 95%, 96%, 97%, 98%, 99%, or 100% identity to any one of SEQ ID NOs: 272-275. In some embodiments, the hinge region comprises an amino acid sequence having at least 80%, 85%, 90%, 95%, 96%, 97%, 98%, 99%, or 100% identity to any one of SEQ ID NOs 265-271. In some embodiments, the CH2 and CH3 domains together comprise an amino acid sequence having at least 80%, 85%, 90%, 95%, 96%, 97%, 98%, 99%, or 100% identity to any one of SEQ ID NOs 229-234.

In some embodiments, the second polypeptide chain comprises, in an N-terminal to C-terminal direction, the VH of the Fab, the antibody constant region, an optional peptide linker, and a second binding domain specific for VEGFA (e.g., any of the VEGFA specific binding domains provided herein which can be comprised in a single polypeptide chain, such as a VHH or in an scFv format). In some embodiments, the optional peptide linker is absent. In some embodiments, the optional peptide linker is present. In some embodiments, the optional peptide linker is present and comprises a sequence of any one of SEQ ID NOs: 21-30. In some embodiments, the optional peptide linker is present and comprises the sequence of SEQ ID NO: 30.

In some embodiments, the second binding domain is a VHH. In some embodiments, the second binding domain VHH comprises a VH CD1, VH CDR2, and VH CDR3 of any one of the anti-VEGFA VHH antibodies described herein (e.g., any of the antibodies described in Table 2B). In some embodiments, the VHH comprises one or more modifications which improve immunogenicity or reduce binding of pre-existing antibodies to the VHH (e.g., a C-terminal “PP” sequence or other such modification described herein). In some embodiments, wherein the VHH comprises a two-proline peptide on its C-terminus. In some embodiments, the VHH comprises a) a VH CDR1 sequence of AYPMM (SEQ ID NO: 202), a VH CDR2 sequence of EISPSGSYTYYADSVRG (SEQ ID NO: 203), and a VH CDR3 sequence of DPRKLDY (SEQ ID NO: 204); b) a VH CDR1 sequence of LYDMM (SEQ ID NO: 206), a VH CDR2 sequence of FIGGDGLNTYYADSVKG (SEQ ID NO: 207), and a VH CDR3 sequence of AGTQFDY (SEQ ID NO: 208); c) a VH CDR1 sequence of WYPMW (SEQ ID NO: 210), a VH CDR2 sequence of LIEGQGDRTYYADSVKG (SEQ ID NO: 211), and a VH CDR3 sequence of AGDRTAGSRGNSFDY (SEQ ID NO: 212); d) a VH CDR1 sequence of AYPMM (SEQ ID NO: 202), a VH CDR2 sequence of EISPSGSYTYYADSVKG (SEQ ID NO: 215), and a VH CDR3 sequence of DPRKFDY (SEQ ID NO: 216); or e) a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220).

In some embodiments, the second binding domain comprises an amino acid sequence having at least 80%, 85%, 90%, 95%, 96%, 97%, 98%, 99% or 100% identity to any one of the VHH antibodies provided in Table 2B (preferably wherein the CDRs are retained). In some embodiments, the second binding domain comprises an amino acid sequence having at least 80%, 85%, 90%, 95%, 96%, 97%, 98%, 99% or 100% identity to any one of SEQ ID NOs: 200, 201, 205, 209, 213, 217, 221, or 280-284 (preferably wherein the CDRs are retained). In some embodiments, the second binding domain comprises an amino acid sequence having at least 80%, 85%, 90%, 95%, 96%, 97%, 98%, 99% or 100% identity to SEQ ID NO: 200, 217, or 221 (preferably wherein the CDRs are retained). In some embodiments, the second binding domain comprises the amino acid sequence set forth in SEQ ID NO: 200, 217, or 221.

In some embodiments, the second polypeptide chain further comprises an additional binding domain targeting VEGFA. In some embodiments, the additional binding domain is a VHH. In some embodiments, the additional binding domain VHH comprises the same CDRs as the second binding domain. In some embodiments, the additional binding domain VHH comprises an identical amino acid sequence compared to the second binding domain. In some embodiments, the additional binding domain VHH comprises the same amino acid sequence as the second binding domain, plus additional amino acids on its C-terminus (e.g., a series of 1, 2, or 3 prolines). In some embodiments, the additional binding domain comprises a C-terminal two-proline peptide which is not present on the second binding domain. In some embodiments, the additional binding domain consists of the same amino acid sequence as the second binding domain. In some embodiments, both the second binding domain and the additional binding domain targeting VEGFA are both VHHs comprising an identical amino acid sequence, or wherein the VHH positioned C-terminal to the other VHH comprises an additional two-proline peptide on its C-terminus as compared to the other VHH, optionally wherein the VHH comprises a two-proline peptide on its C-terminus. In some embodiments, the additional binding domain targeting VEGFA is a different VHH compared to the second binding domain.

In some embodiments, the additional binding domain targeting VEGFA comprises a VH CD1, VH CDR2, and VH CDR3 of any one of the anti-VEGFA VHH antibodies described herein (e.g., any of the antibodies described in Table 2B). In some embodiments, the additional binding domain targeting VEGFA comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the additional binding domain targeting VEGFA comprises an amino acid sequence having at least 80%, 85%, 90%, 95%, 96%, 97%, 98%, 99% or 100% identity to SEQ ID NO: 200, 217, or 221. In some embodiments, the additional binding domain targeting VEGFA comprises the amino acid sequence set forth in SEQ ID NO: 200, 217, or 221.

In some embodiments, the second polypeptide chain comprises, in an N-terminal to C-terminal direction, the VH of the Fab, an antibody constant region, an optional peptide linker, the second binding domain, a second optional peptide linker, and the additional binding domain targeting VEGFA. In some embodiments, the optional peptide linker and the second optional peptide linker are both present and independently selected from any one of SEQ ID NOs: 21-30. In some embodiments, the optional peptide linker is present and comprises the amino acid sequence of SEQ ID NO: 30. In some embodiments, the second optional peptide linker is present and comprises the amino acid sequence of SEQ ID NO: 22. In some embodiments, the optional peptide linker and the second optional peptide linker are both present and comprise the amino acid sequences of SEQ ID NOs: 30 and 22 respectively.

In some embodiments, the second polypeptide chain does not comprise the second binding domain (e.g., the second binding domain and any additional binding domains targeting VEGFA are present on the third polypeptide chain).

In some embodiments, the second polypeptide chain comprises the amino acid sequence set forth in any one of SEQ ID NOs:163, 164, 167, or 169. In some embodiments, the second polypeptide chain comprises the amino acid sequence of SEQ ID N: 163 or 164 with C-terminal modification (e.g., the addition of a two-proline peptide to the C-terminus or another immunogenicity or pre-existing antibody mitigation modification described herein).

In some embodiments, the third polypeptide chain comprises, in an N-terminal to C-terminal direction, the IL-2 polypeptide, an optional peptide linker, and an antibody constant region. In some embodiments, the optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the optional peptide linker is present and has the sequence set forth in SEQ ID NO: 22. In some embodiments, the third polypeptide chain does not comprise the second binding domain or any binding domain targeting VEGFA.

In some embodiments, the third polypeptide chain comprises, in an N-terminal to C-terminal direction, the IL-2 polypeptide, the optional peptide linker, the antibody constant region, a second optional peptide linker, and the second binding domain (i.e., the second polypeptide chain does not comprise the second binding domain or any binding domain targeting VEGFA). In some embodiments, the second optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the second optional peptide linker is present and has the sequence set forth in SEQ ID NO: 22.

The IL-2 polypeptide can be any IL-2 polypeptide described herein (e.g., in any preceding or foregoing section). In some embodiments, the IL-2 polypeptide is one of any one of SEQ ID NOs: 704-774, or a variant thereof. In some embodiments, the IL-2 polypeptide is one of SEQ ID NO: 751. In some embodiments, the IL-2 polypeptide is one of SEQ ID NO: 753. In some embodiments, the IL-2 polypeptide is one of SEQ ID NO: 754. In some embodiments, the IL-2 polypeptide is one of SEQ ID NO: 758.

In some embodiments, the second binding domain present on the third polypeptide chain is a VHH. In some embodiments, the second binding domain VHH comprises a VH CD1, VH CDR2, and VH CDR3 of any one of the anti-VEGFA VHH antibodies described herein (e.g., any of the antibodies described in Table 2B). In some embodiments, the VHH comprises one or more modifications which improve immunogenicity or reduce binding of pre-existing antibodies to the VHH (e.g., a C-terminal “PP” sequence or other such modification described herein). In some embodiments, wherein the VHH comprises a two-proline peptide on its C-terminus. In some embodiments, the VHH comprises a) a VH CDR1 sequence of AYPMM (SEQ ID NO: 202), a VH CDR2 sequence of EISPSGSYTYYADSVRG (SEQ ID NO: 203), and a VH CDR3 sequence of DPRKLDY (SEQ ID NO: 204); b) a VH CDR1 sequence of LYDMM (SEQ ID NO: 206), a VH CDR2 sequence of FIGGDGLNTYYADSVKG (SEQ ID NO: 207), and a VH CDR3 sequence of AGTQFDY (SEQ ID NO: 208); c) a VH CDR1 sequence of WYPMW (SEQ ID NO: 210), a VH CDR2 sequence of LIEGQGDRTYYADSVKG (SEQ ID NO: 211), and a VH CDR3 sequence of AGDRTAGSRGNSFDY (SEQ ID NO: 212); d) a VH CDR1 sequence of AYPMM (SEQ ID NO: 202), a VH CDR2 sequence of EISPSGSYTYYADSVKG (SEQ ID NO: 215), and a VH CDR3 sequence of DPRKFDY (SEQ ID NO: 216); or e) a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the second binding domain comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220).

In some embodiments, the second binding domain present on the third polypeptide chain comprises an amino acid sequence having at least 80%, 85%, 90%, 95%, 96%, 97%, 98%, 99% or 100% identity to any one of the VHH antibodies provided in Table 2B (preferably wherein the CDRs are retained). In some embodiments, the second binding domain comprises an amino acid sequence having at least 80%, 85%, 90%, 95%, 96%, 97%, 98%, 99% or 100% identity to any one of SEQ ID NOs: 200, 201, 205, 209, 213, 217, 221, or 280-284 (preferably wherein the CDRs are retained). In some embodiments, the second binding domain comprises an amino acid sequence having at least 80%, 85%, 90%, 95%, 96%, 97%, 98%, 99% or 100% identity to SEQ ID NO: 200, 217, or 221 (preferably wherein the CDRs are retained). In some embodiments, the second binding domain comprises the amino acid sequence set forth in SEQ ID NO: 200, 217, or 221.

In some embodiments, the third polypeptide chain comprises the second binding domain and an additional binding domain targeting VEGFA. In some embodiments, the additional binding domain targeting VEGFA is positioned C-terminal relative to the second binding domain. In some embodiments, the third polypeptide chain comprises, in an N-terminal to C-terminal direction the IL-2 polypeptide, the optional peptide linker, the antibody constant region, a second optional peptide linker, and the additional binding domain targeting VEGFA. In some embodiments, the second optional peptide linker is present and has a sequence of any one of SEQ ID NOs: 21-30. In some embodiments, the second optional peptide linker is present and has the sequence set forth in SEQ ID NO: 22. In some embodiments, the additional binding domain is a VHH. In some embodiments, the additional binding domain VHH comprises the same CDRs as the second binding domain. In some embodiments, the additional binding domain VHH comprises an identical amino acid sequence compared to the second binding domain. In some embodiments, the additional binding domain VHH comprises the same amino acid sequence as the second binding domain, plus additional amino acids on its C-terminus (e.g., a series of 1, 2, or 3 prolines). In some embodiments, the additional binding domain comprises a C-terminal two-proline peptide which is not present on the second binding domain. In some embodiments, the additional binding domain consists of the same amino acid sequence as the second binding domain. In some embodiments, both the second binding domain and the additional binding domain targeting VEGFA are both VHHs comprising an identical amino acid sequence, or wherein the VHH positioned C-terminal to the other VHH comprises an additional two-proline peptide on its C-terminus as compared to the other VHH, optionally wherein the VHH comprises a two-proline peptide on its C-terminus. In some embodiments, the additional binding domain targeting VEGFA is a different VHH compared to the second binding domain.

In some embodiments, the third polypeptide chain comprises, in an N-terminal to C-terminal direction, the IL-2 polypeptide, the optional peptide linker, the antibody constant region, a second optional peptide linker, and an additional binding domain targeting VEGFA (i.e., the second polypeptide chain does comprise the second binding domain targeting VEGFA). In some embodiments, the second optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a GGGGS (SEQ ID NO: 21), sequence (GGGGS)₂ (SEQ ID NO: 22), (GGGGS)₃ (SEQ ID NO: 29), or (GGGGS)₄ (SEQ ID NO: 30). In some embodiments, the second optional peptide linker is present and has the sequence set forth in SEQ ID NO: 22. In some embodiments, the additional binding domain is a VHH. In some embodiments, the additional binding domain VHH comprises the same CDRs as the second binding domain. In some embodiments, the additional binding domain VHH comprises an identical amino acid sequence compared to the second binding domain. In some embodiments, the additional binding domain VHH comprises the same amino acid sequence as the second binding domain, plus additional amino acids on its C-terminus (e.g., a series of 1, 2, or 3 prolines). In some embodiments, the additional binding domain comprises a C-terminal two-proline peptide which is not present on the second binding domain. In some embodiments, the additional binding domain consists of the same amino acid sequence as the second binding domain. In some embodiments, both the second binding domain and the additional binding domain targeting VEGFA are both VHHs comprising an identical amino acid sequence, or wherein the VHH positioned C-terminal to the other VHH comprises an additional two-proline peptide on its C-terminus as compared to the other VHH, optionally wherein the VHH comprises a two-proline peptide on its C-terminus. In some embodiments, the additional binding domain targeting VEGFA is a different VHH compared to the second binding domain.

In some embodiments, the additional binding domain targeting VEGFA on the third polypeptide chain (either in series with the second binding domain also present on the third polypeptide chain or with the second binding domain present on the second polypeptide chain) comprises a VH CD1, VH CDR2, and VH CDR3 of any one of the anti-VEGFA VHH antibodies described herein (e.g., any of the antibodies described in Table 2B). In some embodiments, the additional binding domain targeting VEGFA comprises a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220). In some embodiments, the additional binding domain targeting VEGFA comprises an amino acid sequence having at least 80%, 85%, 90%, 95%, 96%, 97%, 98%, 99% or 100% identity to a VHH provided in Table 2B (preferably wherein the CDRs are retained). In some embodiments, the additional binding domain targeting VEGFA comprises an amino acid sequence having at least 80%, 85%, 90%, 95%, 96%, 97%, 98%, 99% or 100% identity to SEQ ID NO: 200, 217, or 221. In some embodiments, the additional binding domain targeting VEGFA comprises the amino acid sequence set forth in SEQ ID NO: 200, 217, or 221.

In some embodiments, the antibody constant region of the third polypeptide chain is an IgG1 or IgG4 constant region, or a portion thereof. In some embodiments, the antibody constant region comprises, in an N-terminal to C-terminal direction, a hinge region, a CH2 domain, and a CH3 domain. In some embodiments the antibody constant region comprises, in an N-terminal to C-terminal direction, a hinge region portion, a CH2 domain, and a CH3 domain. In some embodiments, the hinge region comprises an amino acid sequence having at least 80%, 85%, 90%, 95%, 96%, 97%, 98%, 99%, or 100% identity to any one of SEQ ID NOs 265-271. In some embodiments, the CH2 and CH3 domains together comprise an amino acid sequence having at least 80%, 85%, 90%, 95%, 96%, 97%, 98%, 99%, or 100% identity to any one of SEQ ID NOs: 229-234.

In some embodiments, the third polypeptide chain comprises an amino acid sequence as set forth in any one of SEQ ID NOs: 802, 803, 806, or 807.

In some embodiments, the first polypeptide chain comprises the amino acid sequence set forth in SEQ ID NO: 47; the second polypeptide chain comprises the amino acid sequence of any one of SEQ ID NOs: 163, 164, 169, or 171; and the third polypeptide chain comprises the amino acid sequence of any one of SEQ ID NOs: 802, 803, 806, or 807.

In some embodiments, the immunocytokine composition comprises a first polypeptide chain, a second polypeptide chain, and a third polypeptide chain having amino acid sequences, respectively, of
SEQ ID NOs: 47, 163, and 802;
SEQ ID NOs: 47, 163, and 803;
SEQ ID NOs: 47, 164, and 806;
SEQ ID NOs: 47, 164, and 807;
SEQ ID NOs: 47, 169, and 802;
SEQ ID NOs: 47, 169, and 803;
SEQ ID NOs: 47, 172, and 802;
SEQ ID NOs: 47, 172, and 803;
SEQ ID NOs: 47, 163, and 806;
SEQ ID NOs: 47, 163, and 807;
SEQ ID NOs: 47, 169, and 806;
SEQ ID NOs: 47, 169, and 807;
SEQ ID NOs: 47, 171, and 809; or
SEQ ID NOs: 47, 171, and 810.

In some embodiments, the immunocytokine composition comprises a first polypeptide chain, a second polypeptide chain, and a third polypeptide chain having amino acid sequences, respectively, of
SEQ ID NOs: 47, 163, and 802;
SEQ ID NOs: 47, 163, and 803;
SEQ ID NOs: 47, 164, and 806;
SEQ ID NOs: 47, 164, and 807;
SEQ ID NOs: 47, 172, and 802;
SEQ ID NOs: 47, 172, and 803;
SEQ ID NOs: 47, 169, and 802;
SEQ ID NOs: 47, 169, and 803;
SEQ ID NOs: 47, 171, and 809; or
SEQ ID NOs: 47, 171, and 810.

Exemplary constructs are also provided in Table 15

TABLE 15
Exemplary Constructs

| | First | | |
|---|---|---|---|
| | Polypeptide | | |
| | Chain | | |
| | (Anti-PD-1 | Second Polypeptide | Third Polypeptide |
| Molecule | VL) | Chain (Anti-PD-1 VH) | Chain (IL-2) |
| Molecule 1 | SEQ ID | QVQLVQSGVEVKKPGASVKVS | APASSSTKKTQLQLDHLLL |
| | NO: 47 | CKASGYTFTNYYMYWVRQAP | DLQMILNGINNYKNPKLTR |
| | | GQGLEWMGGINPSNGGTNFNE | MLTFKFYMPKKATELKHL |
| | | KFKNRVTLTTDSSTTTAYMEL | QCLEEELKPLEEVLNLAGD |
| | | KSLQFDDTAVYYCARRDYRFD | GSINDLISDINVIVLELKGSE |
| | | MGFDYWGQGTTVTVSSASTKG | TTFMCEYADETATIVEFLN |
| | | PSVFPLAPSSKSTSGGTAALGC | RWITFSQSIISTLTGGGGSG |
| | | LVKDYFPEPVTVSWNSGALTS | GGGSDKTHTCPPCPAPEAA |
| | | GVHTFPAVLQSSGLYSLSSVVT | GGPSVFLFPPKPKDTLMISR |
| | | VPSSSLGTQTYICNVNHKPSNT | TPEVTCVVVDVSHEDPEV |
| | | KVDKKVEPKSCDKTHTCPPCP | KFNWYVDGVEVHNAKTK |
| | | APEAAGGPSVFLFPPKPKDTLM | PREEQYNSTYRVVSVLTVL |
| | | ISRTPEVTCVVVDVSHEDPEVK | HQDWLNGKEYKCKVSNK |
| | | FNWYVDGVEVHNAKTKPREE | ALPAPIEKTISKAKGQPREP |
| | | QYNSTYRVVSVLTVLHQDWLN | QVYTLPPCRDELTKNQVSL |
| | | GKEYKCKVSNKALPAPIEKTIS | WCLVKGFYPSDIAVEWES |
| | | KAKGQPREPQVCTLPPSRDELT | NGQPENNYKTTPPVLDSD |
| | | KNQVSLSCAVKGFYPSDIAVE | GSFFLYSKLTVDKSRWQQ |
| | | WESNGQPENNYKTTPPVLDSD | GNVFSCSVMHEALHNHYT |
| | | GSFFLVSKLTVDKSRWQQGNV | QKSLSLSPG (SEQ ID NO: |
| | | FSCSVMHEALHNHYTQKSLSLS | 802) |
| | | PGKGGGGSGGGGSGGGGSGG | |
| | | GGSDVQLVESGGGLVQPGGSL | |
| | | RLSCAASGRTFSSYSMGWFRQ | |
| | | APGKEREFVVAISKGGYKYDA | |
| | | VSLEGRFTISRDNAKNTVYLQI | |
| | | NSLRPEDTAVYYCASSRAYGSS | |
| | | RLRLADTYEYWGQGTLVTVSS | |
| | | GGGGSGGGGSDVQLVESGGGL | |
| | | VQPGGSLRLSCAASGRTFSSYS | |
| | | MGWFRQAPGKEREFVVAISKG | |
| | | GYKYDAVSLEGRFTISRDNAK | |
| | | NTVYLQINSLRPEDTAVYYCAS | |
| | | SRAYGSSRLRLADTYEYWGQG | |
| | | TLVTVSS (SEQ ID NO: 163) | |
| Molecule 2 | SEQ ID | QVQLVQSGVEVKKPGASVKVS | SEQ ID NO: 802 |
| | NO: 47 | CKASGYTFTNYYMYWVRQAP | |
| | | GQGLEWMGGINPSNGGTNFNE | |
| | | KFKNRVTLTTDSSTTTAYMEL | |
| | | KSLQFDDTAVYYCARRDYRFD | |
| | | MGFDYWGQGTTVTVSSASTKG | |
| | | PSVFPLAPSSKSTSGGTAALGC | |
| | | LVKDYFPEPVTVSWNSGALTS | |
| | | GVHTFPAVLQSSGLYSLSSVVT | |
| | | VPSSSLGTQTYICNVNHKPSNT | |
| | | KVDKKVEPKSCDKTHTCPPCP | |
| | | APEAAGGPSVFLFPPKPKDTLM | |
| | | ISRTPEVTCVVVDVSHEDPEVK | |
| | | FNWYVDGVEVHNAKTKPREE | |
| | | QYNSTYRVVSVLTVLHQDWLN | |
| | | GKEYKCKVSNKALPAPIEKTIS | |
| | | KAKGQPREPQVCTLPPSRDELT | |
| | | KNQVSLSCAVKGFYPSDIAVE | |
| | | WESNGQPENNYKTTPPVLDSD | |
| | | GSFFLVSKLTVDKSRWQQGNV | |
| | | FSCSVMHEALHNHYTQKSLSLS | |
| | | PGKGGGGSGGGGSGGGGSGG | |
| | | GGSDVQLVESGGGLVQPGGSL | |
| | | RLSCAASGRTFSSYSMGWFRQ | |
| | | APGKEREFVVAISKGGYKYDA | |
| | | VSLEGRFTISRDNAKNTVYLQI | |
| | | NSLRPEDTAVYYCASSRAYGSS | |
| | | RLRLADTYEYWGQGTLVTVSS | |
| | | PPGGGGSGGGGSDVQLVESGG | |
| | | GLVQPGGSLRLSCAASGRTFSS | |
| | | YSMGWFRQAPGKEREFVVAIS | |
| | | KGGYKYDAVSLEGRFTISRDN | |
| | | AKNTVYLQINSLRPEDTAVYY | |
| | | CASSRAYGSSRLRLADTYEYW | |
| | | GQGTLVTVSSPP (SEQ ID NO: | |
| | | 169) | |
| Molecule 3 | SEQ ID | QVQLVQSGVEVKKPGASVKVS | SEQ ID NO: 802 |
| | NO: 47 | CKASGYTFTNYYMYWVRQAP | |
| | | GQGLEWMGGINPSNGGTNFNE | |
| | | KFKNRVTLTTDSSTTTAYMEL | |
| | | KSLQFDDTAVYYCARRDYRFD | |
| | | MGFDYWGQGTTVTVSSASTKG | |
| | | PSVFPLAPSSKSTSGGTAALGC | |
| | | LVKDYFPEPVTVSWNSGALTS | |
| | | GVHTFPAVLQSSGLYSLSSVVT | |
| | | VPSSSLGTQTYICNVNHKPSNT | |
| | | KVDKKVEPKSCDKTHTCPPCP | |
| | | APEAAGGPSVFLFPPKPKDTLM | |
| | | ISRTPEVTCVVVDVSHEDPEVK | |
| | | FNWYVDGVEVHNAKTKPREE | |
| | | QYNSTYRVVSVLTVLHQDWLN | |
| | | GKEYKCKVSNKALPAPIEKTIS | |
| | | KAKGQPREPQVCTLPPSRDELT | |
| | | KNQVSLSCAVKGFYPSDIAVE | |
| | | WESNGQPENNYKTTPPVLDSD | |
| | | GSFFLVSKLTVDKSRWQQGNV | |
| | | FSCSVMHEALHNHYTQKSLSLS | |
| | | PGKGGGGSGGGGSGGGGSGG | |
| | | GGSDVQLVESGGGLVQPGGSL | |
| | | RLSCAASGRTFSSYSMGWFRQ | |
| | | APGKEREFVVAISKGGYKYDA | |
| | | VSLEGRFTISRDNAKNTVYLQI | |
| | | NSLRPEDTAVYYCASSRAYGSS | |
| | | RLRLADTYEYWGQGTLVTVSS | |
| | | GGGGSGGGGSDVQLVESGGGL | |
| | | VQPGGSLRLSCAASGRTFSSYS | |
| | | MGWFRQAPGKEREFVVAISKG | |
| | | GYKYDAVSLEGRFTISRDNAK | |
| | | NTVYLQINSLRPEDTAVYYCAS | |
| | | SRAYGSSRLRLADTYEYWGQG | |
| | | TLVTVSSPP (SEQ ID NO: 172) | |
| Molecule 4 | SEQ ID | SEQ ID NO: 163 | APASSSTKKTQLQLEHLLD |
| | NO: 47 | | DLQMILNGINNYKNPKLTR |
| | | | MLTFKFYMPKKATELKHL |
| | | | QCLEEELKPLEEVLNLAGD |
| | | | GSINDLISDINVIVLELKGSE |
| | | | TTFMCEYADETATIVEFLN |
| | | | RWITFSQSIISTLTGGGGSG |
| | | | GGGSDKTHTCPPCPAPEAA |
| | | | GGPSVFLFPPKPKDTLMISR |
| | | | TPEVTCVVVDVSHEDPEV |
| | | | KFNWYVDGVEVHNAKTK |
| | | | PREEQYNSTYRVVSVLTVL |
| | | | HQDWLNGKEYKCKVSNK |
| | | | ALPAPIEKTISKAKGQPREP |
| | | | QVYTLPPCRDELTKNQVSL |
| | | | WCLVKGFYPSDIAVEWES |
| | | | NGQPENNYKTTPPVLDSD |
| | | | GSFFLYSKLTVDKSRWQQ |
| | | | GNVFSCSVMHEALHNHYT |
| | | | QKSLSLSPG (SEQ ID NO: |
| | | | 803) |
| Molecule 5 | (SEQ ID | SEQ ID NO: 169 | SEQ ID NO: 803 |
| | NO: 47) | | |
| | (PL394) | | |
| Molecule 6 | (SEQ ID | SEQ ID NO: 172 | SEQ ID NO: 803 |
| | NO: 47) | | |
| | (PL394) | | |
| Molecule 7 | SEQ ID | QVQLVQSGVEVKKPGASVKVS | APASSSTKKTQLQLDHLLL |
| | NO: 47 | CKASGYTFTNYYMYWVRQAP | DLQMILNGINNYKNPKLTR |
| | | GQGLEWMGGINPSNGGTNFNE | MLTFKFYMPKKATELKHL |
| | | KFKNRVTLTTDSSTTTAYMEL | QCLEEELKPLEEVLNLAGD |
| | | KSLQFDDTAVYYCARRDYRFD | GSINDLISDINVIVLELKGSE |
| | | MGFDYWGQGTTVTVSSASTKG | TTFMCEYADETATIVEFLN |
| | | PSVFPLAPSSKSTSGGTAALGC | RWITFSQSIISTLTGGGGSG |
| | | LVKDYFPEPVTVSWNSGALTS | GGGSDKTHTCPPCPAPEAA |
| | | GVHTFPAVLQSSGLYSLSSVVT | GGPSVFLFPPKPKDTLMISR |
| | | VPSSSLGTQTYICNVNHKPSNT | TPEVTCVVVDVSHEDPEV |
| | | KVDKKVEPKSCDKTHTCPPCP | KFNWYVDGVEVHNAKTK |
| | | APEAAGGPSVFLFPPKPKDTLM | PREEQYNSTYRVVSVLTVL |
| | | ISRTPEVTCVVVDVSHEDPEVK | HQDWLNGKEYKCKVSNK |
| | | FNWYVDGVEVHNAKTKPREE | ALPAPIEKTISKAKGQPREP |
| | | QYNSTYRVVSVLTVLHQDWLN | QVYTLPPCRDELTKNQVSL |
| | | GKEYKCKVSNKALPAPIEKTIS | WCLVKGFYPSDIAVEWES |
| | | KAKGQPREPQVCTLPPSRDELT | NGQPENNYKTTPPVLDSD |
| | | KNQVSLSCAVKGFYPSDIAVE | GSFFLYSKLTVDKSRWQQ |
| | | WESNGQPENNYKTTPPVLDSD | GNVFSCSVMHEALHNHYT |
| | | GSFFLVSKLTVDKSRWQQGNV | QKSLSLSPGKGGGGSGGG |
| | | FSCSVMHEALHNHYTQKSLSLS | GSGGGGSGGGGSDVQLVE |
| | | PGKGGGGSGGGGSGGGGSGG | SGGGLVQPGGSLRLSCAAS |
| | | GGSDVQLVESGGGLVQPGGSL | GRTFSSYSMGWFRQAPGK |
| | | RLSCAASGRTFSSYSMGWFRQ | EREFVVAISKGGYKYDAVS |
| | | APGKEREFVVAISKGGYKYDA | LEGRFTISRDNAKNTVYLQ |
| | | VSLEGRFTISRDNAKNTVYLQI | INSLRPEDTAVYYCASSRA |
| | | NSLRPEDTAVYYCASSRAYGSS | YGSSRLRLADTYEYWGQG |
| | | RLRLADTYEYWGQGTLVTVSS | TLVTVSSPP (SEQ ID NO: |
| | | PP (SEQ ID NO: 171) | 809) |
| Molecule 8 | SEQ ID | SEQ ID NO: 171 | APASSSTKKTQLQLEHLLD |
| | NO: 47 | | DLQMILNGINNYKNPKLTR |
| | | | MLTFKFYMPKKATELKHL |
| | | | QCLEEELKPLEEVLNLAGD |
| | | | GSINDLISDINVIVLELKGSE |
| | | | TTFMCEYADETATIVEFLN |
| | | | RWITFSQSIISTLTGGGGSG |
| | | | GGGSDKTHTCPPCPAPEAA |
| | | | GGPSVFLFPPKPKDTLMISR |
| | | | TPEVTCVVVDVSHEDPEV |
| | | | KFNWYVDGVEVHNAKTK |
| | | | PREEQYNSTYRVVSVLTVL |
| | | | HQDWLNGKEYKCKVSNK |
| | | | ALPAPIEKTISKAKGQPREP |
| | | | QVYTLPPCRDELTKNQVSL |
| | | | WCLVKGFYPSDIAVEWES |
| | | | NGQPENNYKTTPPVLDSD |
| | | | GSFFLYSKLTVDKSRWQQ |
| | | | GNVFSCSVMHEALHNHYT |
| | | | QKSLSLSPGKGGGGSGGG |
| | | | GSGGGGSGGGGSDVQLVE |
| | | | SGGGLVQPGGSLRLSCAAS |
| | | | GRTFSSYSMGWFRQAPGK |
| | | | EREFVVAISKGGYKYDAVS |
| | | | LEGRFTISRDNAKNTVYLQ |
| | | | INSLRPEDTAVYYCASSRA |
| | | | YGSSRLRLADTYEYWGQG |
| | | | TLVTVSSPP (SEQ ID NO: |
| | | | 810) (PL812) |

The table 16 below provides additional embodiments of potential immunocytokine compositions described herein. For each entry, the portions are arranged from N- to —C terminus going down from the top of the table for each polypeptide chain.

TABLE 16
Exemplary Constructs (any combination of the referenced sequences)

| | Sequence | Embodiment | Embodiment | Embodiment | Embodiment | Embodiment | Embodiment |
|---|---|---|---|---|---|---|---|
| Chain | Portion | A1 | A2 | A3 | A4 | A5 | A6 |
| First | PD-1 VL | 49, 51, | 49, 51, | 49, 51, | 49, 51, | 49, 51, | 49, 51, |
| Polypeptide | SEQ | 73, or 77 | 73, or 77 | 73, or 77 | 73, or 77 | 73, or 77 | 73, or 77 |
| Chain | ID NOs | | | | | | |
| | Light | 276, 277, | 276, 277, | 276, 277, | 276, 277, | 276, 277, | 276, 277, |
| | Chain | or 278 | or 278 | or 278 | or 278 | or 278 | or 278 |
| | Constant | | | | | | |
| | Domain | | | | | | |
| | SEQ | | | | | | |
| | ID Nos | | | | | | |
| Second | PD-1 VH | 48, 50, | 48, 50, | 48, 50, | 48, 50, | 48, 50, | 48, 50, |
| Polypeptide | SEQ | 72, or 76 | 72, or 76 | 72, or 76 | 72, or 76 | 72, or 76 | 72, or 76 |
| Chain | ID Nos | | | | | | |
| | CH1 | 272, 273, | 272, 273, | 272, 273, | 272, 273, | 272, 273, | 272, 273, |
| | SEQ | 274, or | 274, or | 274, or | 274, or | 274, or | 274, or |
| | ID Nos | 275 | 275 | 275 | 275 | 275 | 275 |
| | Hinge | 265-271 | 265-271 | 265-271 | 265-271 | 265-271 | 265-271 |
| | SEQ | | | | | | |
| | ID Nos | | | | | | |
| | CH2-CH3 | 229-234 | 229-234 | 229-234 | 229-234 | 229-234 | 229-234 |
| | SEQ | | | | | | |
| | ID Nos | | | | | | |
| | Optional | 21-30 | 21-30 | Absent | 21-30 | 21-30 | 21-30 |
| | Linker | | | | | | |
| | SEQ | | | | | | |
| | ID Nos | | | | | | |
| | VEGF | 200, 201, | 200, 201, | Absent | 217 or | 200 | 217 or |
| | VHH | 205, 209, | 205, 209, | | 221 | | 221 |
| | SEQ | 213, 213, | 213, 213, | | | | |
| | ID Nos | 217, 221, | 217, 221, | | | | |
| | | 280-285 | 280-285 | | | | |
| | Optional | 21-30 | Absent | Absent | 21-30 | Absent | 21-30 |
| | Linker | | | | | | |
| | SEQ | | | | | | |
| | ID Nos | | | | | | |
| | VEGF | 200, 201, | Absent | Absent | 200, 217, | Absent | 200 |
| | VHH | 205, 209, | | | or 221 | | |
| | SEQ | 213, 213, | | | | | |
| | ID Nos | 217, 221, | | | | | |
| | | 280-285 | | | | | |
| Third | IL-2 | 704-774 | 704-774 | 704-774 | 751, 753, | 751, 753, | 751, 753, |
| Polypeptide | SEQ | | | | 754, or | 754, or | 754, or |
| Chain | ID Nos | | | | 758 | 758 | 758 |
| | Optional | 21-30 | 21-30 | 22-30 | 21-30 | 21-30 | 21-30 |
| | Linker | | | | | | |
| | SEQ | | | | | | |
| | ID Nos | | | | | | |
| | Hinge | 265-271 | 265-271 | 265-271 | 265-271 | 265-271 | 265-271 |
| | SEQ | | | | | | |
| | ID Nos | | | | | | |
| | CH2-CH3 | 229-234 | 229-234 | 229-234 | 229-234 | 229-234 | 229-234 |
| | SEQ | | | | | | |
| | ID Nos | | | | | | |
| | Optional | Absent | 21-30 | 21-30 | Absent | 21-30 | Absent |
| | Linker | | | | | | |
| | SEQ | | | | | | |
| | ID Nos | | | | | | |
| | VEGF | Absent | 200, 201, | 200, 201, | Absent | 200 | Absent |
| | VHH | | 205, 209, | 205, 209, | | | |
| | SEQ | | 213, 213, | 213, 213, | | | |
| | ID Nos | | 217, 221, | 217, 221, | | | |
| | | | 280-285 | 280-285 | | | |
| | Optional | Absent | Absent | 22-30 | Absent | Absent | Absent |
| | Linker | | | | | | |
| | SEQ | | | | | | |
| | ID Nos | | | | | | |
| | VEGF | Absent | Absent | 200, 201, | Absent | Absent | Absent |
| | VHH | | | 205, 209, | | | |
| | SEQ | | | 213, 213, | | | |
| | ID Nos | | | 217, 221, | | | |
| | | | | 280-285 | | | |

### Exemplary Fusion Immunocytokine Compositions Having an Anti-VEGFA Fab and a N-Terminal Fused IL-2

In the following section are exemplary formats of immunocytokine compositions described herein prepared as fusion proteins which comprise a Fab domain targeting VEGFA (e.g., any of the Fab domains described herein, such as a Fab domain of an anti-VEGFA antibody such as Bevacizumab, or another suitable anti-VEGFA antibody), one or more binding domains targeting PD-1 (e.g., any of the VHH domains targeting PD-1 described herein, such as VHH147) and an IL-2 polypeptide (e.g., any of the IL-2 polypeptides described herein) fused via its N-terminus to an Fc domain opposite the Fc domain (e.g., as depicted in FIG. 8C or FIG. 8D). In some embodiments described in this section, the formats described are those depicted in FIG. 8C or FIG. 8D. In the version depicted in FIG. 8C with the two anti-PD-1 VHH domains positioned in series, each anti-PD-1 VHH domain can be separated by a peptide linker. Analogous formats and the other formats described herein are contemplated as also within the scope of the instant disclosure. Throughout this section, the “first polypeptide chain” refers to a polypeptide comprising the VL of the anti-VEGF Fab, the “second polypeptide chain” refers to a polypeptide comprising the VH of the anti-VEGF Fab, and the “third polypeptide chain” refers to the polypeptide comprising the IL-2 polypeptide. In embodiments wherein the third polypeptide chain contains another VH of another anti-VEGF Fab (e.g., a copy of the first anti-VEGF fab of the first and second polypeptide chains), an additional copy of the first polypeptide chain will be present on the molecule. Analogous constructs in which the VEGFA and PD-1 binding domains are swapped (e.g., any of the constructs described in this section, but each PD-1 binding domain is swapped for an analogous VEGFA binding domain and vice versa) are also contemplated herein.

In some embodiments herein is an immunocytokine composition in which the second binding domain is specific for VEGFA and comprises a Fab having a VH and a VL, and wherein the composition comprises: a) a first polypeptide chain comprising the VL of the first binding domain; b) a second polypeptide chain comprising the VH of the first binding domain; and c) a third polypeptide chain comprising the IL-2 polypeptide.

In some embodiments, the second binding domain Fab comprises a VH having a VH CD1, VH CDR2, and VH CDR3 and a VL having a VL CDR1, CDR2, and CDR3 of any one of the anti-VEGFA antibodies described herein (e.g., any of the antibodies described in Table 2A, such as Bevacizumab). In some embodiments, the first binding domain Fab comprises a VH and a VL of any one of the anti-VEGFA antibodies described herein (e.g., any of the antibodies described in Table 2A, such as Bevacizumab), or a sequence having at least 80%, 85%, 90%, 95%, 96%, 97%, 98%, or 99% identity thereto (preferably wherein the CDRs are retained). In some embodiments, the first polypeptide chain comprises, in N- to C-terminal direction, the VL and a light chain constant region. In some embodiments, the light chain constant region comprises an amino acid having at least 80%, 85%, 90%, 95%, 96%, 97%, 98%, 99%, or 100% sequence identity to SEQ ID NO: 276, 277, or 278.

In some embodiments, the second polypeptide chain comprises, in an N-terminal to C-terminal direction, the VH of the Fab of the second binding domain and an antibody constant region. In some embodiments, the antibody constant region is an IgG1 or an IgG4. In some embodiments, the IgG1 or IgG4 comprises an amino acid sequence having at least 90%, 95%, 96%, 97%, 98%, 99%, or 100% identity to a wild type IgG1 or IgG4 sequence.

In some embodiments the antibody constant region comprises, in an N-terminal to C-terminal direction, a CH1 domain, a hinge region, a CH2 domain, and a CH3 domain. In some embodiments, the CH1 domain comprises an amino acid sequence having at least 80%, 85%, 90%, 95%, 96%, 97%, 98%, 99%, or 100% identity to any one of SEQ ID NOs: 272-275. In some embodiments, the hinge region comprises an amino acid sequence having at least 80%, 85%, 90%, 95%, 96%, 97%, 98%, 99%, or 100% identity to any one of SEQ ID NOs 265-271. In some embodiments, the CH2 and CH3 domains together comprise an amino acid sequence having at least 80%, 85%, 90%, 95%, 96%, 97%, 98%, 99%, or 100% identity to any one of SEQ ID NOs 229-234.

In some embodiments, the second polypeptide chain comprises, in an N-terminal to C-terminal direction, the VH of the Fab, the antibody constant region, an optional peptide linker, and a first binding domain specific for PD-1 (e.g., any of the PD-1 specific binding domains provided herein which can be comprised in a single polypeptide chain, such as a VHH or in an scFv format). In some embodiments, the optional peptide linker is absent. In some embodiments, the optional peptide linker is present. In some embodiments, the optional peptide linker is present and comprises a sequence of any one of SEQ ID NOs: 21-30. In some embodiments, the optional peptide linker is present and comprises the sequence of SEQ ID NO: 30.

In some embodiments, the first binding domain is a VHH. In some embodiments, the first binding domain VHH comprises a VH CDR1, VH CDR2, and VH CDR3 of any one of the anti-PD-1 VHH antibodies described herein (e.g., any of the antibodies described in Table 1B or Table 1C, such as VHH47, VHH62, VHH70, VHH76, VHH84, or VHH178). In some embodiments, the first binding domain VHH comprises a VH CDR1, VH CDR2, and VH CDR3 of VHH47. In some embodiments, the VHH comprises one or more modifications which improve immunogenicity or reduce binding of pre-existing antibodies to the VHH (e.g., a C-terminal “PP” sequence or other such modification described herein). In some embodiments, wherein the VHH comprises a two-proline peptide on its C-terminus.

In some embodiments, the first binding domain comprises an amino acid sequence having at least 80%, 85%, 90%, 95%, 96%, 97%, 98%, 99% or 100% identity to any one of the VHH antibodies provided in Table 1C (preferably wherein the CDRs are retained). In some embodiments, the first binding domain comprises an amino acid sequence having at least 80%, 85%, 90%, 95%, 96%, 97%, 98%, 99% or 100% identity to any one of VHH 47, VHH62, VHH70, VHH76, VHH84, or VHH178. In some embodiments, the first binding domain comprises an amino acid sequence having at least 80%, 85%, 90%, 95%, 96%, 97%, 98%, 99% or 100% identity to any one of VHH 47, VHH62, VHH70, VHH76, VHH84, or VHH178, wherein the CDRs are retained. In some embodiments, the first binding domain is VHH 47, VHH62, VHH70, VHH76, VHH84, or VHH178.

In some embodiments, the second polypeptide chain further comprises an additional binding domain targeting PD-1. In some embodiments, the additional binding domain is a VHH. In some embodiments, the additional binding domain VHH comprises the same CDRs as the first binding domain. In some embodiments, the additional binding domain VHH comprises an identical amino acid sequence compared to the first binding domain. In some embodiments, the additional binding domain VHH comprises the same amino acid sequence as the first binding domain, plus additional amino acids on its C-terminus (e.g., a series of 1, 2, or 3 prolines). In some embodiments, the additional binding domain comprises a C-terminal two-proline peptide which is not present on the first binding domain. In some embodiments, the additional binding domain consists of the same amino acid sequence as the second binding domain. In some embodiments, both the second binding domain and the additional binding domain targeting PD-1 are both VHHs comprising an identical amino acid sequence, or wherein the VHH positioned C-terminal to the other VHH comprises an additional two-proline peptide on its C-terminus as compared to the other VHH, optionally wherein the VHH comprises a two-proline peptide on its C-terminus. In some embodiments, the additional binding domain targeting PD-1 is a different VHH compared to the second binding domain.

In some embodiments, the additional binding domain targeting PD-1 comprises a VH CD1, VH CDR2, and VH CDR3 of any one of the anti-PD-1 VHH antibodies described herein (e.g., any of the antibodies described in Table 1C). In some embodiments, additional binding domain targeting PD-1 comprises a VH CD1, VH CDR2, and VH CDR3 of any one of VHH47, VHH62, VHH70, VHH76, VHH84, or VHH178. In some embodiments, the additional binding domain targeting PD-1 comprises an amino acid sequence having at least 80%, 85%, 90%, 95%, 96%, 97%, 98%, 99% or 100% identity to VHH47, VHH62, VHH70, VHH76, VHH84, or VHH178. In some embodiments, the additional binding domain targeting PD-1 is VHH47, VHH62, VHH70, VHH76, VHH84, or VHH178.

In some embodiments, the second polypeptide chain comprises, in an N-terminal to C-terminal direction, the VH of the Fab, an antibody constant region, an optional peptide linker, the second binding domain, a second optional peptide linker, and the additional binding domain targeting VEGFA. In some embodiments, the optional peptide linker and the second optional peptide linker are both present and independently selected from any one of SEQ ID NOs: 21-30. In some embodiments, the optional peptide linker is present and comprises the amino acid sequence of SEQ ID NO: 30. In some embodiments, the second optional linker is present and comprises the amino acid sequence of SEQ ID NO: 22. In some embodiments, the optional peptide linker and the second optional peptide linker are both present and comprise the amino acid sequences of SEQ ID NOs: 30 and 22 respectively.

In some embodiments, the third polypeptide chain comprises, in an N-terminal to C-terminal direction, a VH of a second Fab specific for VEGFA, an antibody constant region, an optional peptide linker, and the IL-2 polypeptide.

In some embodiments, the second Fab comprises a VH having a VH CD1, VH CDR2, and VH CDR3 and a VL having a VL CDR1, CDR2, and CDR3 of any one of the anti-VEGFA antibodies described herein (e.g., any of the antibodies described in Table 2A).

In some embodiments, the second Fab comprises a VH and a VL of any one of the anti-VEGFA antibodies described herein (e.g., any of the antibodies described in Table 2A), or a sequence having at least 80%, 85%, 90%, 95%, 96%, 97%, 98%, or 99% identity thereto (preferably wherein the CDRs are retained). In some embodiments, the second Fab is the same as the first Fab.

In some embodiments, the antibody constant region of the third polypeptide is an IgG1 or an IgG4. In some embodiments, the IgG1 or IgG4 comprises an amino acid sequence having at least 90%, 95%, 96%, 97%, 98%, 99%, or 100% identity to a wild type IgG1 or IgG4 sequence.

In some embodiments the antibody constant region of the third polypeptide comprises, in an N-terminal to C-terminal direction, a CH1 domain, a hinge region, a CH2 domain, and a CH3 domain. In some embodiments, the CH1 domain comprises an amino acid sequence having at least 80%, 85%, 90%, 95%, 96%, 97%, 98%, 99%, or 100% identity to any one of SEQ ID NOs: 272-275. In some embodiments, the hinge region comprises an amino acid sequence having at least 80%, 85%, 90%, 95%, 96%, 97%, 98%, 99%, or 100% identity to any one of SEQ ID NOs 265-271. In some embodiments, the CH2 and CH3 domains together comprise an amino acid sequence having at least 80%, 85%, 90%, 95%, 96%, 97%, 98%, 99%, or 100% identity to any one of SEQ ID NOs 229-234.

In some embodiments, the optional peptide linker is present and has a sequence (GS)ₙ (SEQ ID NO: 23), (GGS)ₙ (SEQ ID NO: 24), (GGGS)ₙ (SEQ ID NO: 25), (GGSG)ₙ (SEQ ID NO: 26), (GGSGG)ₙ (SEQ ID NO: 27), (GGGGS)ₙ (SEQ ID NO: 28), wherein n is 1, 2, 3, 4, 5, 6, 7, 8, 9, or 10, or a sequence GGGGS (SEQ ID NO: 21), (GGGGS)₂ (SEQ ID NO: 22), (GGGGS)₃ (SEQ ID NO: 29), or (GGGGS)₄ (SEQ ID NO: 30). In some embodiments, the optional peptide linker is present and has the sequence set forth in SEQ ID NO: 29.

The IL-2 polypeptide can be any IL-2 polypeptide described herein (e.g., in any preceding or foregoing section). In some embodiments, the IL-2 polypeptide is one of any one of SEQ ID NOs: 704-774, or a variant thereof. In some embodiments, the IL-2 polypeptide is one of SEQ ID NO: 751. In some embodiments, the IL-2 polypeptide is one of SEQ ID NO: 753. In some embodiments, the IL-2 polypeptide is one of SEQ ID NO: 754. In some embodiments, the IL-2 polypeptide is one of SEQ ID NO: 758.

### Exemplary Immunocytokine Compositions of the Format of FIG. 6B

Non-limiting examples of immunocytokine compositions according to the instant disclosure are described in the Table below. Each Composition in the Table 17 below is of the format shown in FIG. 6B and comprises a light chain polypeptide having the sequence of SEQ ID NO: 47 (comprising the VL of Pembrolizumab); a first heavy chain polypeptide having the sequence of QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSSHEDPEVKFNWYVDGVEVHNAK TKPREEQYNSTYRVVSVLTVLHQDWLNGKEYKCKVSNKALPAPIEKTISKAKGQPREPQ VCTLPPSRDELTKNQVSLSCAVKGFYPSDIAVEWESNGQPENNYKTTPPVLDSDGSFFLV SKLTVDKSRWQQGNVFSCSVMHEALHNHYTQKSLSLSPGKGGGGSGGGGSGGGGSGG GGSDVQLVESGGGLVQPGGSLRLSCAASGRTFSSYSMGWFRQAPGKEREFVVAISKGG YKYDAVSLEGRFTISRDNAKNTVYLQINSLRPEDTAVYYCASSRAYGSSRLRLADTYEY WGQGTLVTVSS (SEQ ID NO: 164) (comprising the VH of Pembrolizumab and VHH175 provided herein), and a second heavy chain polypeptide (which contains VHH175 provided herein) having the indicated IL-2 polypeptide fused to the N-terminus of the sequence DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPCRDELTKNQVSLWCLVKGFYPSDIAVEWESNGQPENNYKTTP PVLDSDGSFFLYSKLTVDKSRWQQGNVFSCSVMHEALHNHYTQKSLSLSPGKGGGGSG GGGSGGGGSGGGGSDVQLVESGGGLVQPGGSLRLSCAASGRTFSSYSMGWFRQAPGKE REFVVAISKGGYKYDAVSLEGRFTISRDNAKNTVYLQINSLRPEDTAVYYCASSRAYGS SRLRLADTYEYWGQGTLVTVSS (SEQ ID NO: 801) via a (GGGGS)₂ linker (SEQ ID NO: 22). For example, Composition 62 has a light chain polypeptide of SEQ ID NO: 47, a first heavy chain polypeptide of SEQ ID NO: 164, and a second heavy chain polypeptide having the sequence APASSSTKKTQLQLDHLLLDLQMILNGINNYKNPKLTRMLTFKFYMPKKATELKHLQCL EEELKPLEEVLNLAGDGSINDLISDINVIVLELKGSETTFMCEYADETATIVEFLNRWITFS QSIISTLTGGGGSGGGGSDKTHTCPPCPAPEAAGGPSVFLFPPKPKDTLMISRTPEVTCVV VDVSHEDPEVKFNWYVDGVEVHNAKTKPREEQYNSTYRVVSVLTVLHQDWLNGKEY KCKVSNKALPAPIEKTISKAKGQPREPQVYTLPPCRDELTKNQVSLWCLVKGFYPSDIAV EWESNGQPENNYKTTPPVLDSDGSFFLYSKLTVDKSRWQQGNVFSCSVMHEALHNHYT QKSLSLSPGKGGGGSGGGGSGGGGSGGGGSDVQLVESGGGLVQPGGSLRLSCAASGRT FSSYSMGWFRQAPGKEREFVVAISKGGYKYDAVSLEGRFTISRDNAKNTVYLQINSLRP EDTAVYYCASSRAYGSSRLRLADTYEYWGQGTLVTVSS (SEQ ID NO: 806) and Composition 66 has a light chain polypeptide of SEQ ID NO: 47, a first heavy chain polypeptide of SEQ ID NO: 164, and a second heavy chain polypeptide having the sequence APASSSTKKTQLQLEHLLDDLQMILNGINNYKNPKLTRMLTFKFYMPKKATELKHLQCL EEELKPLEEVLNLAGDGSINDLISDINVIVLELKGSETTFMCEYADETATIVEFLNRWITFS QSIISTLTGGGGSGGGGSDKTHTCPPCPAPEAAGGPSVFLFPPKPKDTLMISRTPEVTCVV VDVSHEDPEVKFNWYVDGVEVHNAKTKPREEQYNSTYRVVSVLTVLHQDWLNGKEY KCKVSNKALPAPIEKTISKAKGQPREPQVYTLPPCRDELTKNQVSLWCLVKGFYPSDIAV EWESNGQPENNYKTTPPVLDSDGSFFLYSKLTVDKSRWQQGNVFSCSVMHEALHNHYT QKSLSLSPGKGGGGSGGGGSGGGGSGGGGSDVQLVESGGGLVQPGGSLRLSCAASGRT FSSYSMGWFRQAPGKEREFVVAISKGGYKYDAVSLEGRFTISRDNAKNTVYLQINSLRP EDTAVYYCASSRAYGSSRLRLADTYEYWGQGTLVTVSS (SEQ ID NO: 807). Also provided herein are analogous compositions to Compositions 62 and 66 (or any of the other compositions provided in the table below) in which the second heavy polypeptide chain comprises a C-terminal modification which reduces immunogenicity or pre-existing antibody binding, such as the additional of a proline-proline dipeptide to the sequence containing the VHH (e.g., SEQ ID NO: 806 or 807). It is expressly contemplated that the compositions described below could be modified to incorporate any of the PD-1 binding Fabs herein, an alternative VEGFA binding V described herein, an alternative Fc domain described herein, an alternative peptide linker described herein, or any combination thereof.

TABLE 17
Exemplary Immunocytokines of the Format of FIG. 6B

| | IL-2 | | IL-2 |
|---|---|---|---|
| Composition | Payload | | SEQ |
| No. | Name | IL-2 Modifications | ID NO |
| 1 | 2P1 | IL2_T3A_F42A_Y45A_L72G_C125A | 704 |
| 2 | 2P2 | IL2_T3A_Y31H_K35R_Q74P_N88D_C125A | 705 |
| 3 | 2P3 | IL2_T3A_N88R_S130R_IL15B′C′ loop | 706 |
| 6 | 2P4 | IL2_T3A_N88D_C125S | 707 |
| 7 | 2P5 | IL2_T3A_N88R_C125S | 708 |
| 8 | 2P6 | IL2_T3A_D20V_C125S | 709 |
| 9 | 2P7 | IL2_T3A_H16S_N88D_C125S | 710 |
| 10 | 2P8 | IL2_T3A_D20V_V91A_C125S | 711 |
| 11 | 2P9 | IL2_T3A_E15S_N88D_C125S | 712 |
| 12 | 2P10 | IL2_T3A_E15S_Q22T_V91L_C125S | 713 |
| 13 | 2P11 | IL2_T3A_E15S_Q22T_N88D_C125S | 714 |
| 14 | 2P12 | IL2_T3A_N88D_C125S_Q126T | 715 |
| 15 | 2P13 | IL2_T3A_N88D_C125S_I129A | 716 |
| 16 | 2P14 | IL2_T3A_N88D_C125S_I129L | 717 |
| 17 | 2P15 | IL2_T3A_E15S_N88D_C125S_I129A | 718 |
| 18 | 2P16 | IL2_T3A_E15S_N88D_C125S_I129L | 719 |
| 19 | 2P17 | IL2_T3A_N88D_C125S_I129K | 720 |
| 20 | 2P18 | IL2_T3A_E15S_H16S_N88D_C125S | 721 |
| 21 | 2P19 | IL2_T3A_Q22T_N88D_E95S_C125S | 722 |
| 22 | 2P20 | IL2_T3A_Q22T_S87A_N88D_C125S | 723 |
| 23 | 2P21 | IL2_T3A_E15S_D84K_N88D_C125S | 724 |
| 24 | 2P22 | IL2_T3A_E15S_Q22T_S87A_N88D_C125S | 725 |
| 25 | 2P23 | IL2_T3A_E15S_N88D_C125S_Q126T | 726 |
| 26 | 2P24 | IL2_T3A_N88R_C125S_S130R | 727 |
| 27 | 2P25 | IL2_T3A_F42A_N88R_C125S_S130R_IL15B′C′ loop | 728 |
| 28 | 2P26 | IL2_T3A_N88R_C125S_S130R_GDGSIN B′C′ loop | 729 |
| 29 | 2P27 | IL2_T3A_K76A_R81S_N88R_C125S_S130R | 730 |
| 30 | 2P28 | IL2_T3A_K76A_R81S_R83S_N88R_C125S_S130R | 731 |
| 31 | 2P29 | IL2_T3A_N88R_C125S_S130R_QSGH AB loop | 732 |
| 32 | 2P30 | IL2_T3A_K32S_K35E_R38A_N88R_C125S_S130R | 733 |
| 33 | 2P31 | IL2_T3A_N88R_I92L_C125S_S130R | 734 |
| 34 | 2P32 | IL2_T3A_E15S_N88D_T123A_C125S_I129A | 735 |
| 35 | 2P33 | IL2_T3A_N88D_T123A_C125S | 736 |
| 36 | 2P34 | IL2_T3A_L12A_E15S_L19A_C125S | 737 |
| 37 | 2P35 | IL2_T3A_L12A_E15S_L19D_C125S | 738 |
| 38 | 2P36 | IL2_T3A_L12Y_E15S_L19A_C125S | 739 |
| 39 | 2P37 | IL2_T3A_L12Y_L19D_C125S | 740 |
| 40 | 2P38 | IL2_T3A_E15D_N88D_C125S | 741 |
| 41 | 2P39 | IL2_T3A_L12A_E15S_L19A_N88D_C125S | 742 |
| 42 | 2P40 | IL2_T3A_L12A_E15S_L19D_N88D_C125S | 743 |
| 43 | 2P41 | IL2_T3A_L12Y_E15S_L19A_N88D_C125S | 744 |
| 44 | 2P42 | IL2_T3A_L12Y_L19D_N88D_C125S | 745 |
| 45 | 2P43 | IL2_T3A_L12V_L19V_N88D_C125S | 746 |
| 46 | 2P44 | IL2_T3A_L12A_L19A_N88D_C125S | 747 |
| 47 | 2P45 | IL2_T3A_L12Y_N88D_C125S | 748 |
| 48 | 2P46 | IL2_T3A_L19D_N88D_C125S | 749 |
| 49 | 2P47 | IL2_T3A_Y31H_K35R_Q74P_N88D_C125A_Q126T | 750 |
| 59 | 2P48 | IL2_T3A_N88D_C125S_Q126T_GDGSIN B′C′ loop | 751 |
| 60 | 2P49 | IL2_T3A_N88D_C125S_I129K_GDGSIN B′C′ loop | 752 |
| 61 | 2P50 | IL2_T3A_E15S_N88D_T123A_C125S_I129A_GDGSIN | 753 |
| | | B′C′ loop | |
| 62 | 2P51 | IL2_T3A_E15D_N88D_C125S_GDGSIN B′C′ loop | 754 |
| 63 | 2P52 | IL2_T3A_L12A_E15S_L19A_N88D_C125S_GDGSIN | 755 |
| | | B′C′ loop | |
| 64 | 2P53 | IL2_T3A_L12Y_L19D_N88D_C125S_GDGSIN | 756 |
| | | B′C′ loop | |
| 65 | 2P54 | IL2_T3A_L12A_L19A_N88D_C125S_GDGSIN | 757 |
| | | B′C′ loop | |
| 66 | 2P55 | IL2_T3A_L19D_N88D_C125S_GDGSIN B′C′ loop | 758 |
| 67 | 2P56 | IL2_T3A_K76A_R81S_N88D_C125S_Q126T | 759 |
| 68 | 2P57 | IL2_T3A_K76A_R81S_N88D_C125S_I129K | 760 |
| 69 | 2P58 | IL2_T3A_E15S_K76A_R81S_N88D_T123A_C125S_I129A | 761 |
| 70 | 2P59 | IL2_T3A_E15D_K76A_R81S_N88D_C125S | 762 |
| 71 | 2P60 | IL2_T3A_L12A_E15S_L19A_K76A_R81S_N88D_C125S | 763 |
| 72 | 2P61 | IL2_T3A_L12Y_L19D_K76A_R81S_N88D_C125S | 764 |
| 73 | 2P62 | IL2_T3A_L12A_L19A_K76A_R81S_N88D_C125S | 765 |
| 74 | 2P63 | IL2_T3A_L19D_K76A_R81S_N88D_C125S | 766 |
| 75 | 2P64 | IL2_T3A_K32S_K35E_N88D_C125S_Q126T | 767 |
| 76 | 2P65 | IL2_T3A_K32S_K35E_N88D_C125S_I129K | 768 |
| 77 | 2P66 | IL2_T3A_E15S_K32S_K35E_N88D_T123A_C125S_I129A | 769 |
| 78 | 2P67 | IL2_T3A_E15D_K32S_K35E_N88D_C125S | 770 |
| 79 | 2P68 | IL2_T3A_L12A_E15S_L19A_K32S_K35E_N88D_C125S | 771 |
| 80 | 2P69 | IL2_T3A_L12Y_L19D_K32S_K35E_N88D_C125S | 772 |
| 81 | 2P70 | IL2_T3A_L12A_L19A_K32S_K35E_N88D_C125S | 773 |
| 82 | 2P71 | IL2_T3A_L19D_K32S_K35E_N88D_C125S | 774 |

### Exemplary Immunocytokine Compositions of the Format of FIG. 8C

Non-limiting examples of immunocytokine compositions according to the instant disclosure are described in the Table below. Each Composition in the Table 18 below is of the format shown in FIG. 8C and comprises a light chain polypeptide having the sequence of SEQ ID NO: 285 (comprising the VL of Bevacizumab and an L154K modification); a first heavy chain polypeptide having the sequence of EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQPR EPQVCTLPPSRDELTKNQVSLSCAVKGFYPSDIAVEWESNGQPENNYKTTPPVLDSDGSF FLVSKLTVDKSRWQQGNVFSCSVMHEALHNHYTQKSL SLSPGKGGGGSGGGGSGGGG SGGGGSDVQLVESGGGLVQPGGSLRLSCAASGLPFSDYSMGWFRQAPGKEREFVAGIS GSSITTYYADSVKGRFTISRDNSKNTLYLQMNSLRPEDTAVYYCATSGYSYVAGGMDV WGQGTTVTVSSGGGGSGGGGSGGGGSDVQLVESGGGLVQPGGSLRLSCAASGLPFSDY SMGWFRQAPGKEREFVAGISGSSITTYYADSVKGRFTISRDNSKNTLYLQMNSLRPEDT AVYYCATSGYSYVAGGMDVWGQGTTVTVSS (SEQ ID NO: 166) (comprising the VH of Bevacizumab and two copies of anti-PD-1 VHH 47 described herein), and a second heavy chain polypeptide having the indicated IL-2 polypeptide fused to the C-terminus of the sequence EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSSHEDPEVKFNWYVDGVEVHN AKTKPREEQYNSTYRVVSVLTVLHQDWLNGKEYKCKVSNKALPAPIEKTISKAKGQPR EPQVYTLPPCRDELTKNQVSLWCLVKGFYPSDIAVEWESNGQPENNYKTTPPVLDSDGS FFLYSKLTVDKSRWQQGNVFSCSVMHEALHNHYTQKSLSLSPGK (SEQ ID NO: 168) via a (GGGGS)₃ linker (SEQ ID NO: 29). For example, Composition 98 comprises a light chain polypeptide of SEQ ID NO: 285, a first heavy chain polypeptide of SEQ ID NO: 166, and a third polypeptide chain of the sequence EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQPR EPQVYTLPPCRDELTKNQVSLWCLVKGFYPSDIAVEWESNGQPENNYKTTPPVLDSDGS FFLYSKLTVDKSRWQQGNVFSCSVMHEALHNHYTQKSLSLSPGKGGGGSGGGGSGGG GSAPASSSTKKTQLQLEHLLDDLQMILNGINNYKNPKLTRMLTFKFYMPKKATELKHLQ CLEEELKPLEEVLNLAGDGSINDLISDINVIVLELKGSETTFMCEYADETATIVEFLNRWIT FSQSIISTLT (SEQ ID NO: 804) and Composition 94 comprises a light chain polypeptide of SEQ ID NO: 285, a first heavy chain polypeptide of SEQ ID NO: 166, and a third polypeptide chain of the sequence EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSSHEDPEVKFNWYVDGVEVHN AKTKPREEQYNSTYRVVSVLTVLHQDWLNGKEYKCKVSNKALPAPIEKTISKAKGQPR EPQVYTLPPCRDELTKNQVSLWCLVKGFYPSDIAVEWESNGQPENNYKTTPPVLDSDGS FFLYSKLTVDKSRWQQGNVFSCSVMHEALHNHYTQKSLSLSPGKGGGGSGGGGSGGG GSAPASSSTKKTQLQLDHLLLDLQMILNGINNYKNPKLTRMLTFKFYMPKKATELKHLQ CLEEELKPLEEVLNLAGDGSINDLISDINVIVLELKGSETTFMCEYADETATIVEFLNRWIT FSQSIISTLT (SEQ ID NO: 805). Also provided herein are analogous compositions to Compositions 98 and 94 (or any of the other compositions provided in the table below) in which the first heavy chain polypeptide comprises a C-terminal modification which reduces immunogenicity or pre-existing antibody binding, such as the additional of a proline-proline dipeptide to the C-terminus of the sequence containing the VHH (e.g., SEQ ID NO: 166). It is expressly contemplated that the compositions described below could be modified to incorporate any of the VEGFA binding Fabs herein, an alternative PD-1 binding VHH described herein, an alternative Fc domain sequence, or an alternative peptide linker sequence, or any combination thereof.

TABLE 18
Exemplary Immunocytokines of the Format of FIG. 8C

| | IL-2 | | IL-2 |
|---|---|---|---|
| | Payload | | SEQ |
| Composition | Name | Modifications | ID NO |
| 91 | 2P48 | IL2_T3A_N88D_C125S_Q126T_GDGSIN B′C′ loop | 751 |
| 92 | 2P49 | IL2_T3A_N88D_C125S_I129K_GDGSIN B′C′ loop | 752 |
| 93 | 2P50 | IL2_T3A_E15S_N88D_T123A_C125S_I129A_GDGSIN | 753 |
| | | B′C′ loop | |
| 94 | 2P51 | IL2_T3A_E15D_N88D_C125S_GDGSIN B′C′ loop | 754 |
| 95 | 2P52 | IL2_T3A_L12A_E15S_L19A_N88D_C125S_GDGSIN | 755 |
| | | B′C′ loop | |
| 96 | 2P53 | IL2_T3A_L12Y_L19D_N88D_C125S_GDGSIN | 756 |
| | | B′C′ loop | |
| 97 | 2P54 | IL2_T3A_L12A_L19A_N88D_C125S_GDGSIN | 757 |
| | | B′C′ loop | |
| 98 | 2P55 | IL2_T3A_L19D_N88D_C125S_GDGSIN B′C′ loop | 758 |
| 99 | 2P56 | IL2_T3A_K76A_R81S_N88D_C125S_Q126T | 759 |
| 100 | 2P57 | IL2_T3A_K76A_R81S_N88D_C125S_I129K | 760 |
| 101 | 2P58 | IL2_T3A_E15S_K76A_R81S_N88D_T123A_C125S_I129A | 761 |
| 102 | 2P59 | IL2_T3A_E15D_K76A_R81S_N88D_C125S | 762 |
| 103 | 2P60 | IL2_T3A_L12A_E15S_L19A_K76A_R81S_N88D_C125S | 763 |
| 104 | 2P61 | IL2_T3A_L12Y_L19D_K76A_R81S_N88D_C125S | 764 |
| 105 | 2P62 | IL2_T3A_L12A_L19A_K76A_R81S_N88D_C125S | 765 |
| 106 | 2P63 | IL2_T3A_L19D_K76A_R81S_N88D_C125S | 766 |
| 107 | 2P64 | IL2_T3A_K32S_K35E_N88D_C125S_Q126T | 767 |
| 108 | 2P65 | IL2_T3A_K32S_K35E_N88D_C125S_I129K | 768 |
| 109 | 2P66 | IL2_T3A_E15S_K32S_K35E_N88D_T123A_C125S_I129A | 769 |
| 110 | 2P67 | IL2_T3A_E15D_K32S_K35E_N88D_C125S | 770 |
| 111 | 2P68 | IL2_T3A_L12A_E15S_L19A_K32S_K35E_N88D_C125S | 771 |
| 112 | 2P69 | IL2_T3A_L12Y_L19D_K32S_K35E_N88D_C125S | 772 |
| 113 | 2P70 | IL2_T3A_L12A_L19A_K32S_K35E_N88D_C125S | 773 |
| 114 | 2P71 | IL2_T3A_L19D_K32S_K35E_N88D_C125S | 774 |
| 115 | 2P3 | IL2_T3A_N88R_S130R_IL15B′C′ loop | 706 |

Additional exemplary immunocytokine compositions in the format depicted in FIG. 8C include those described in the table below. Also provided herein are analogous compositions to those provided in the table below in which the first heavy chain polypeptide comprises a C-terminal modification which reduces immunogenicity or pre-existing antibody binding, such as the additional of a proline-proline dipeptide to the C-terminus of the sequence containing the VHH.

TABLE 19
Additional Immunocytokines in the Format of FIG. 8C

| | | | | | Heavy | Heavy |
|---|---|---|---|---|---|---|
| | | | | | Chain 1 | Chain 2 |
| | | | | | Sequence | Sequence |
| | | | | Light | with VHHs | with IL-2 |
| Composition | VEGF | PD-1 | | Chain | (N-to C- | (N-to C- |
| Number | Binder | Binder | IL-2 | Sequence | terminus) | terminus) |
| 55 | Bevacizumab | Reference | 2P3 | SEQ ID | SEQ ID NO: | SEQ ID |
| | Fab | anti-PD1 | | NO: 285 | 161; | NO: 168; |
| | | VHH A | | | SEQ ID NO: | SEQ ID |
| | | | | | 29; [Reference | NO: 29; |
| | | | | | anti-PD1 | SEQ ID |
| | | | | | VHH A]; | NO: 706 |
| | | | | | SEQ ID NO: | |
| | | | | | 22: [Reference | |
| | | | | | anti-PD1 | |
| | | | | | VHH A] | |
| 87 | Bevacizumab | anti-PD1 | 2P55 | SEQ ID | SEQ ID NO: | SEQ ID |
| | Fab | VHH62 | | NO: 285 | 161; SEQ ID | NO: 168; |
| | | | | | NO: 29; SEQ | SEQ ID |
| | | | | | ID NO: 569; | NO: 29; |
| | | | | | SEQ ID NO: | SEQ ID |
| | | | | | 22; SEQ ID | NO: 758 |
| | | | | | NO: 569 | |
| 88 | Bevacizumab | anti-PD1 | 2P55 | SEQ ID | SEQ ID NO: | SEQ ID |
| | Fab | VHH70 | | NO: 285 | 161; SEQ ID | NO: 168; |
| | | | | | NO: 29; SEQ | SEQ ID |
| | | | | | ID NO: 601; | NO: 29; |
| | | | | | SEQ ID NO: | SEQ ID |
| | | | | | 22: SEQ ID | NO: 758 |
| | | | | | NO: 601 | |
| 89 | Bevacizumab | anti-PD1 | 2P55 | SEQ ID | SEQ ID NO: | SEQ ID |
| | Fab | VHH76 | | NO: 285 | 161; SEQ ID | NO: 168; |
| | | | | | NO: 29; SEQ | SEQ ID |
| | | | | | ID NO: 625; | NO: 29; |
| | | | | | SEQ ID NO: | SEQ ID |
| | | | | | 22; SEQ ID | NO: 758 |
| | | | | | NO: 625 | |
| 90 | Bevacizumab | anti-PD1 | 2P55 | SEQ ID | SEQ ID NO: | SEQ ID |
| | Fab | VHH84 | | NO: 285 | 161; SEQ ID | NO: 168; |
| | | | | | NO: 29; SEQ | SEQ ID |
| | | | | | ID NO: 657; | NO: 29; |
| | | | | | SEQ ID NO: | SEQ ID |
| | | | | | 22; SEQ ID | NO: 758 |
| | | | | | NO: 657 | |
| 120 | Bevacizumab | anti-PD1 | 2P48 | SEQ ID | SEQ ID NO: | |
| | Fab | VHH178 | | NO: 285 | 161; SEQ ID | SEQ ID |
| | | | | | NO: 29; SEQ | NO: 168; |
| | | | | | ID NO: 693; | SEQ ID |
| | | | | | SEQ ID NO: | NO: 29; |
| | | | | | 22; SEQ ID | SEQ ID |
| | | | | | NO: 693 | NO: 751 |
| 53 | Bevacizumab | Reference | 2P1 | SEQ ID | SEQ ID NO: | SEQ ID |
| | Fab | anti-PD1 | | NO: 285 | 161; SEQ ID | NO: 168; |
| | | VHH A | | | NO: 29; | SEQ ID |
| | | | | | [Reference anti- | NO: 29; |
| | | | | | PD1 VHH A]; | SEQ ID |
| | | | | | SEQ ID NO: | NO: 704 |
| | | | | | 22; [Reference | |
| | | | | | anti-PD1 | |
| | | | | | VHH A] | |
| 54 | Bevacizumab | Reference | 2P2 | SEQ ID | SEQ ID NO: | SEQ ID |
| | Fab | anti-PD1 | | NO: 285 | 161; SEQ ID | NO: 168; |
| | | VHH A | | | NO: 29; | SEQ ID |
| | | | | | [Reference anti- | NO: 29; |
| | | | | | PD1 VHH A]; | SEQ ID |
| | | | | | SEQ ID NO: | NO: 705 |
| | | | | | 22; [Reference | |
| | | | | | anti-PD1 | |
| | | | | | VHH A] | |

### Exemplary Immunocytokine Compositions of the Format of FIG. 8A

Non-limiting examples of immunocytokine compositions according to the instant disclosure are described in the Table below. Each Composition in the Table 20 below is of the format shown in FIG. 8A and comprises a light chain polypeptide having the sequence of SEQ ID NO: 47 (comprising the VL of Pembrolizumab); a first heavy chain polypeptide having the sequence of APASSSTKKTQLQLDHLLLDLQMILNGINNYKNPKLTRMLTFKFYMPKKATELKHLQCL EEELKPLEEVLNLAGDGSINDLISDINVIVLELKGSETTFMCEYADETATIVEFLNRWITFS QSIISTLTGGGGSGGGGSDKTHTCPPCPAPEAAGGPSVFLFPPKPKDTLMISRTPEVTCVV VDVSHEDPEVKFNWYVDGVEVHNAKTKPREEQYNSTYRVVSVLTVLHQDWLNGKEY KCKVSNKALPAPIEKTISKAKGQPREPQVYTLPPCRDELTKNQVSLWCLVKGFYPSDIAV EWESNGQPENNYKTTPPVLDSDGSFFLYSKLTVDKSRWQQGNVFSCSVMIIHEALHNHYT QKSLSLSPG (SEQ ID NO: 802), which includes the IL-2 polypeptide 2P51 described herein fused to an Fc domain, and a second heavy chain polypeptide having two copies of the anti-VEGFA VHH binding domains as described in the table below fused to the C-terminus of the sequence QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPREPQ VCTLPPSRDELTKNQVSLSCAVKGFYPSDIAVEWESNGQPENNYKTTPPVLDSDGSFFLV SKLTVDKSRWQQGNVFSCSVMIIHEALHNHYTQKSLSLSPGK (SEQ ID NO: 167), with the first copy of the anti-VEGFA VHH fused to the C-terminus of SEQ ID NO: 167 via a (GGGGS)₄ linker (SEQ ID NO: 30) and the second copy of the anti-VEGFA VHH fused to the C-terminus of the first copy of the anti-VEGFA VHH via a (GGGGS)₂ linker (SEQ ID NO: 22). For example, Composition 118 has a light chain as set forth in SEQ ID NO: 47, a first heavy chain polypeptide chain of SEQ ID NO: 802, and a third polypeptide chain having the sequence: QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPREPQ VCTLPPSRDELTKNQVSLSCAVKGFYPSDIAVEWESNGQPENNYKTTPPVLDSDGSFFLV SKLTVDKSRWQQGNVFSCSVMHEALHNHYTQKSLSLSPGKGGGGSGGGGSGGGGSGG GGSDVQLVESGGGLVQPGGSLRLSCAASGRTFSSYSMGWFRQAPGKEREFVVAISKGG YKYDAVSLEGRFTISRDNAKNTVYLQINSLRPEDTAVYYCASSRAYGSSRLRLADTYEY WGQGTLVTVSSPPGGGGSGGGGSDVQLVESGGGLVQPGGSLRLSCAASGRTFSSYSMG WFRQAPGKEREFVVAISKGGYKYDAVSLEGRFTISRDNAKNTVYLQINSLRPEDTAVYY CASSRAYGSSRLRLADTYEYWGQGTLVTVSSPP (SEQ ID NO: 169). Composition 116 has a light chain as set forth in SEQ ID NO: 47, a first heavy chain polypeptide chain of SEQ ID NO: 802, and a third polypeptide chain having the sequence of QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPREPQ VCTLPPSRDELTKNQVSLSCAVKGFYPSDIAVEWESNGQPENNYKTTPPVLDSDGSFFLV SKLTVDKSRWQQGNVFSCSVMHEALHNHYTQKSLSLSPGKGGGGSGGGGSGGGGSGG GGSDVQLVESGGGLVQPGGSLRLSCAASGRTFSSYSMGWFRQAPGKEREFVVAISKGG YKYDAVSLEGRFTISRDNAKNTVYLQINSLRPEDTAVYYCASSRAYGSSRLRLADTYEY WGQGTLVTVSSGGGGSGGGGSDVQLVESGGGLVQPGGSLRLSCAASGRTFSSYSMGW FRQAPGKEREFVVAISKGGYKYDAVSLEGRFTISRDNAKNTVYLQINSLRPEDTAVYYC ASSRAYGSSRLRLADTYEYWGQGTLVTVSS (SEQ ID NO: 163). Also provided herein are analogous compositions to Compositions 116 (or any of the other compositions provided in the table below) in which the first heavy chain polypeptide comprises a C-terminal modification which reduces immunogenicity or pre-existing antibody binding, such as the additional of a proline-proline dipeptide to the C-terminus of the sequence containing the VHH (e.g., SEQ ID NO: 163). It is expressly contemplated that the compositions described below could be modified to incorporate any of the PD1 binding Fabs herein, an alternative IL-2 polypeptide described herein, an alternative Fc domain sequence, or alternative peptide linker sequences, or any combination thereof.

TABLE 20
Exemplary Immunocytokines of the Format of FIG. 8A

| Composition | | |
|---|---|---|
| Number | VHH Name | VHH SEQ ID NO |
| 118 | anti-VEGF | 200 |
| | VHH176 | |
| 121 | anti-VEGF | 300 |
| | VHH127 | |
| 122 | anti-VEGF | 304 |
| | VHH128 | |
| 123 | anti-VEGF | 308 |
| | VHH129 | |
| 124 | anti-VEGF | 312 |
| | VHH130 | |
| 125 | anti-VEGF | 316 |
| | VHH131 | |
| 126 | anti-VEGF | 320 |
| | VHH132 | |
| 127 | anti-VEGF | 324 |
| | VHH133 | |
| 128 | anti-VEGF | 328 |
| | VHH134 | |
| 129 | anti-VEGF | 332 |
| | VHH135 | |
| 130 | anti-VEGF | 336 |
| | VHH136 | |
| 131 | anti-VEGF | 340 |
| | VHH137 | |
| 132 | anti-VEGF | 344 |
| | VHH138 | |
| 133 | anti-VEGF | 348 |
| | VHH139 | |
| 134 | anti-VEGF | 352 |
| | VHH140 | |
| 135 | anti-VEGF | 356 |
| | VHH141 | |
| 136 | anti-VEGF | 360 |
| | VHH142 | |
| 137 | anti-VEGF | 364 |
| | VHH143 | |
| 138 | anti-VEGF | 368 |
| | VHH144 | |
| 139 | anti-VEGF | 372 |
| | VHH145 | |
| 140 | anti-VEGF | 376 |
| | VHH146 | |
| 141 | anti-VEGF | 380 |
| | VHH147 | |
| 142 | anti-VEGF | 384 |
| | VHH148 | |
| 143 | anti-VEGF | 388 |
| | VHH149 | |
| 144 | anti-VEGF | 392 |
| | VHH150 | |
| 145 | anti-VEGF | 396 |
| | VHH151 | |
| 146 | anti-VEGF | 400 |
| | VHH152 | |
| 147 | anti-VEGF | 404 |
| | VHH153 | |
| 148 | anti-VEGF | 408 |
| | VHH154 | |
| 149 | anti-VEGF | 412 |
| | VHH155 | |
| 150 | anti-VEGF | 416 |
| | VHH156 | |
| 151 | anti-VEGF | 420 |
| | VHH157 | |
| 152 | anti-VEGF | 424 |
| | VHH158 | |
| 153 | anti-VEGF | 428 |
| | VHH159 | |
| 154 | anti-VEGF | 432 |
| | VHH160 | |
| 155 | anti-VEGF | 436 |
| | VHH161 | |
| 156 | anti-VEGF | 440 |
| | VHH162 | |
| 157 | anti-VEGF | 444 |
| | VHH163 | |
| 158 | anti-VEGF | 448 |
| | VHH164 | |
| 159 | anti-VEGF | 452 |
| | VHH165 | |
| 160 | anti-VEGF | 456 |
| | VHH166 | |
| 161 | anti-VEGF | 460 |
| | VHH167 | |
| 162 | anti-VEGF | 464 |
| | VHH168 | |
| 163 | anti-VEGF | 468 |
| | VHH169 | |
| 164 | anti-VEGF | 472 |
| | VHH170 | |
| 165 | anti-VEGF | 476 |
| | VHH171 | |
| 166 | anti-VEGF | 480 |
| | VHH172 | |
| 167 | anti-VEGF | 484 |
| | VHH173 | |
| 168 | anti-VEGF | 488 |
| | VHH174 | |
| 116 | anti-VEGF | 221 |
| | VHH175 | |

### Additional Exemplary Immunocytokine Compositions of the Format of FIG. 8A

Non-limiting examples of immunocytokine compositions according to the instant disclosure are described in the Table 21 below. Each Composition in the Table 21 below is of the format shown in FIG. 8A and comprises a light chain polypeptide having the sequence of SEQ ID NO: 47 (comprising the VL of Pembrolizumab); a first heavy chain polypeptide having the sequence of APASSSTKKTQLQLEHLLDDLQMILNGINNYKNPKLTRMLTFKFYMPKKATELKHLQCL EEELKPLEEVLNLAGDGSINDLISDINVIVLELKGSETTFMCEYADETATIVEFLNRWITFS QSIISTLTGGGGSGGGGSDKTHTCPPCPAPEAAGGPSVFLFPPKPKDTLMISRTPEVTCVV VDVSHEDPEVKFNWYVDGVEVHNAKTKPREEQYNSTYRVVSVLTVLHQDWLNGKEY KCKVSNKALPAPIEKTISKAKGQPREPQVYTLPPCRDELTKNQVSLWCLVKGFYPSDIAV EWESNGQPENNYKTTPPVLDSDGSFFLYSKLTVDKSRWQQGNVFSCSVMHEALHNHYT QKSLSLSPG (SEQ ID NO: 803), which includes the IL-2 polypeptide 2P55 described herein fused to an Fc domain, and a second heavy chain polypeptide having two copies of the anti-VEGFA VHH binding domains as described in the table below fused to the C-terminus of the sequence SEQ ID NO: 167, with the first copy of the anti-VEGFA VHH fused to the C-terminus of SEQ ID NO: 167 via a (GGGGS)₄ linker (SEQ ID NO: 30) and the second copy of the anti-VEGFA VHH fused to the C-terminus of the first copy of the anti-VEGFA VHH via a (GGGGS)₂ linker (SEQ ID NO: 22) For example, Composition 119 has a light chain polypeptide of SEQ ID NO: 47, a first heavy chain polypeptide of SEQ ID NO: 803, and a third heavy chain polypeptide of SEQ ID NO: 169 and Composition 117 has a light chain polypeptide of SEQ ID NO: 47, a first heavy chain polypeptide of SEQ ID NO: 803, and a third heavy chain polypeptide of SEQ ID NO: 163. Also provided herein are analogous compositions to Compositions 98 and 94 (or any of the other compositions provided in the table below) in which the second heavy polypeptide chain comprises a C-terminal modification which reduces immunogenicity or pre-existing antibody binding, such as the additional of a proline-proline dipeptide to the C-terminus of the sequence containing the VHH (e.g., SEQ ID NO: 163). It is expressly contemplated that the compositions described below could be modified to incorporate any of the PD1 binding Fabs herein, an alternative IL-2 polypeptide described herein, an alternative Fc domain sequence, or alternative peptide linker sequences, or any combination thereof.

TABLE FIG. 21
Additional Exemplary Immunocytokines of the Format of FIG. 8A

| Composition | | |
|---|---|---|
| Number | VHH Name | VHH SEQ ID NO |
| 119 | anti-VEGF | 200 |
| | VHH176 | |
| 169 | anti-VEGF | 300 |
| | VHH127 | |
| 170 | anti-VEGF | 304 |
| | VHH128 | |
| 171 | anti-VEGF | 308 |
| | VHH129 | |
| 172 | anti-VEGF | 312 |
| | VHH130 | |
| 173 | anti-VEGF | 316 |
| | VHH131 | |
| 174 | anti-VEGF | 320 |
| | VHH132 | |
| 175 | anti-VEGF | 324 |
| | VHH133 | |
| 176 | anti-VEGF | 328 |
| | VHH134 | |
| 177 | anti-VEGF | 332 |
| | VHH135 | |
| 178 | anti-VEGF | 336 |
| | VHH136 | |
| 179 | anti-VEGF | 340 |
| | VHH137 | |
| 180 | anti-VEGF | 344 |
| | VHH138 | |
| 181 | anti-VEGF | 348 |
| | VHH139 | |
| 182 | anti-VEGF | 352 |
| | VHH140 | |
| 183 | anti-VEGF | 356 |
| | VHH141 | |
| 184 | anti-VEGF | 360 |
| | VHH142 | |
| 185 | anti-VEGF | 364 |
| | VHH143 | |
| 186 | anti-VEGF | 368 |
| | VHH144 | |
| 187 | anti-VEGF | 372 |
| | VHH145 | |
| 188 | anti-VEGF | 376 |
| | VHH146 | |
| 189 | anti-VEGF | 380 |
| | VHH147 | |
| 190 | anti-VEGF | 384 |
| | VHH148 | |
| 191 | anti-VEGF | 388 |
| | VHH149 | |
| 192 | anti-VEGF | 392 |
| | VHH150 | |
| 193 | anti-VEGF | 396 |
| | VHH151 | |
| 194 | anti-VEGF | 400 |
| | VHH152 | |
| 195 | anti-VEGF | 404 |
| | VHH153 | |
| 196 | anti-VEGF | 408 |
| | VHH154 | |
| 197 | anti-VEGF | 412 |
| | VHH155 | |
| 198 | anti-VEGF | 416 |
| | VHH156 | |
| 199 | anti-VEGF | 420 |
| | VHH157 | |
| 200 | anti-VEGF | 424 |
| | VHH158 | |
| 201 | anti-VEGF | 428 |
| | VHH159 | |
| 202 | anti-VEGF | 432 |
| | VHH160 | |
| 203 | anti-VEGF | 436 |
| | VHH161 | |
| 204 | anti-VEGF | 440 |
| | VHH162 | |
| 205 | anti-VEGF | 444 |
| | VHH163 | |
| 206 | anti-VEGF | 448 |
| | VHH164 | |
| 207 | anti-VEGF | 452 |
| | VHH165 | |
| 208 | anti-VEGF | 456 |
| | VHH166 | |
| 209 | anti-VEGF | 460 |
| | VHH167 | |
| 210 | anti-VEGF | 464 |
| | VHH168 | |
| 211 | anti-VEGF | 468 |
| | VHH169 | |
| 212 | anti-VEGF | 472 |
| | VHH170 | |
| 213 | anti-VEGF | 476 |
| | VHH171 | |
| 214 | anti-VEGF | 480 |
| | VHH172 | |
| 215 | anti-VEGF | 484 |
| | VHH173 | |
| 216 | anti-VEGF | 488 |
| | VHH174 | |
| 117 | anti-VEGF | 221 |
| | VHH175 | |

Another exemplary immunocytokine described herein in the format provided in FIG. 8A is one having a light chain polypeptide of SEQ ID NO: 47, a first heavy chain polypeptide of SEQ ID NO: 172, and a second heavy chain polypeptide of SEQ ID NO: 802 or 803. Additional Exemplary Fusion Immunocytokine Constructs

The instant disclosure also provides the following exemplary compositions. Analogous compositions in which one or more of the PD1 binding domain(s), the VEGF binding domain(s), the IL-2 polypeptide, the Fc region, the peptide linkers, or any combination thereof of the following exemplary compositions are switched for another analogous feature are also contemplated.

Composition 4 as described herein refers to an anti-PD-1/IL-2 immunocytokine with no VEGFA binding domain. Composition 4 comprises a light chain polypeptide of SEQ ID NO: 47, a first heavy chain polypeptide of SEQ ID NO: 167, and a second heavy chain polypeptide having, in a N-terminal to C-terminal direction, the IL-2 polypeptide 2P1 (modifications: IL2_T3A_F42A_Y45A_L72G_C125A) (SEQ ID NO: 704), a peptide linker having the sequence of SEQ ID NO: 22, and a polypeptide of the sequence DKTHTCPPCPAPEAAGGPSVFLFPPKPKDTLMISRTPEVTCVVVDVSHEDPEVKFNWYV DGVEVHNAKTKPREEQYNSTYRVVSVLTVLHQDWLNGKEYKCKVSNKALPAPIEKTIS KAKGQPREPQVYTLPPCRDELTKNQVSLWCLVKGFYPSDIAVEWESNGQPENNYKTTP PVLDSDGSFFLYSKLTVDKSRWQQGNVFSCSVMTHEALHNHIYTQKSLSLSPG (SEQ ID NO: 808). The anti-PD-1 binding domain of Composition 4 is a single Fab of Pembrolizumab.

Composition 4 and additional compositions are described in the Table 22 below.

TABLE 22
Additional Compositions

| | | | | | | Hole Heavy | Knob Heavy |
|---|---|---|---|---|---|---|---|
| | | | | | | Chain | Chain |
| | | | | | Light | Sequence | Sequence |
| Composition | PD1 | VEGF | IL2 | | Chain | (N- to C- | (N- to C- |
| No. | binder | binder | payload | Format | Sequence | terminus) | terminus) |
| 4 | Pembrolizumab | NA | 2P1 | FIG. 6B | SEQ ID | SEQ ID NO: | SEQ ID |
| | Fab | | (IL2_T3A_ | with anti- | NO: 47 | 167 | NO: 704; |
| | | | F42A_Y45A_ | VHH | | | SEQ ID |
| | | | L72G_C125A) | domains | | | NO: 22; |
| | | | | omitted | | | SEQ ID |
| | | | | | | | NO: 808 |
| 5 | Pembrolizumab | anti-VEGF | N/A | FIG. 6B | SEQ ID | SEQ ID NO: | SEQ ID |
| | Fab | VHH175 | | with IL-2 | NO: 47 | 164 | NO: 21; |
| | | | | omitted | | | SEQ ID |
| | | | | | | | NO: 808; |
| | | | | | | | SEQ ID |
| | | | | | | | NO: SEQ |
| | | | | | | | ID NO: 30; |
| | | | | | | | SEQ ID |
| | | | | | | | NO: 221 |
| 52 | Reference | Bevacizumab | 2P3 | FIG. 8D | SEQ ID | SEQ ID NO: | SEQ ID |
| | anti-PD1 | Fab | (IL2_T3A_ | | NO: 285 | 161; SEQ ID | NO: 168; |
| | VHH A | | N88R_S130R_ | | | NO: 30; | SEQ ID |
| | | | IL15 | | | [Reference | NO: 29; |
| | | | B′C′ loop) | | | anti-PD1 | SEQ ID |
| | | | | | | VHH A] | NO: 706 |
| 56 | Reference | Bevacizumab | 2P3 | FIG. 8E | SEQ ID | SEQ ID NO: | SEQ ID |
| | anti-PD1 | Fab | (IL2_T3A_ | | NO: 285 | 161; SEQ ID | NO: 168; |
| | VHH A | | N88R_S130R_ | | | NO: 30; | SEQ ID |
| | | | IL15 | | | [Reference | NO: 29; |
| | | | B′C′ loop) | | | anti-PD1 | SEQ ID |
| | | | | | | VHH A] | NO: 706; |
| | | | | | | | SEQ ID |
| | | | | | | | NO: 29; |
| | | | | | | | [Reference |
| | | | | | | | anti-PD1 |
| | | | | | | | VHH A] |
| 57 | [Reference | Bevacizumab | 2P3 | FIG. 8F | SEQ ID | SEQ ID NO: | SEQ ID |
| | anti-PD1 | Fab | (IL2_T3A_ | | NO: 285 | 161; SEQ ID | NO: 168; |
| | VHH A] | | N88R_S130R_ | | | NO: 30; | SEQ ID |
| | | | IL15 | | | [Reference | NO: 29; |
| | | | B′C′ loop) | | | anti-PD1 | [Reference |
| | | | | | | VHH A] | anti-PD1 |
| | | | | | | | VHH A]; |
| | | | | | | | SEQ ID |
| | | | | | | | NO: 29; |
| | | | | | | | SEQ ID |
| | | | | | | | NO: 706 |
| 58 | Pembrolizumab | anti-VEGF | 2P3 | FIG. 8G | SEQ ID | SEQ ID NO: | SEQ ID |
| | Fab | VHH175 | (IL2_T3A_ | | NO: 47 | 163 | NO: 706; |
| | | | N88R_S130R_ | | | | SEQ ID |
| | | | IL15 | | | | NO: 22; |
| | | | B′C′ loop) | | | | SEQ ID |
| | | | | | | | NO: 801 |
| 83 | Pembrolizumab | anti-VEGF | 2P3 | FIG. 8A | SEQ ID | SEQ ID NO: | SEQ ID |
| | Fab | VHH175 | (IL2_T3A_ | | NO: 47 | 163 | NO: 706; |
| | | | N88R_S130R_ | | | | SEQ ID |
| | | | IL15 | | | | NO: 22: |
| | | | B′C′ loop) | | | | SEQ ID |
| | | | | | | | NO: 808 |
| 84 | Pembrolizumab | NA | 2P3 | FIG. 6B | SEQ ID | SEQ ID NO: | SEQ ID |
| | Fab | | (IL2_T3A_ | with anti- | NO: 47 | 167 | NO: 706; |
| | | | N88R_S130R_ | VHH | | | SEQ ID |
| | | | IL15 | domains | | | NO: 22: |
| | | | B′C′ loop) | omitted | | | SEQ ID |
| | | | | | | | NO: 808 |
| 85 | Pembrolizumab | anti-VEGF | 2P3 | FIG. 8H | SEQ ID | SEQ ID NO: | SEQ ID |
| | Fab | VHH175 | (IL2_T3A_ | | NO: 47 | 164 | NO: 706; |
| | | | N88R_S130R_ | | | | SEQ ID |
| | | | IL15 | | | | NO: 22: |
| | | | B′C′ loop) | | | | SEQ ID |
| | | | | | | | NO: 808 |
| 50 | Reference | Bevacizumab | 2P1 | FIG. 8D | SEQ ID | SEQ ID NO: | SEQ ID |
| | anti-PD1 | Fab | (IL2_T3A_ | | NO: 285 | 161; SEQ ID | NO: 168; |
| | VHH A | | F42A_Y45A_ | | | NO: 30; | SEQ ID |
| | | | L72G_C125A) | | | [Reference | NO: 29; |
| | | | | | | anti-PD1 | SEQ ID |
| | | | | | | VHH A] | NO: 704 |
| 51 | Reference | Bevacizumab | 2P2 | FIG. 8D | SEQ ID | SEQ ID NO: | SEQ ID |
| | anti-PD1 | Fab | (IL2_T3A_ | | NO: 285 | 161; SEQ ID | NO: 168; |
| | VHH A | | Y31H_K35R_ | | | NO: 30; | SEQ ID |
| | | | Q74P_N88D_ | | | [Reference | NO: 29; |
| | | | C125A) | | | anti-PD1 | SEQ ID |
| | | | | | | VHH A] | NO: 705 |

SEQ ID NO: 161 is EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSTHEDPEVKFNWYVDGVEVHN AKTKPREEQYNSTYRVVSVLTVLHQDWLNGKEYKCKVSNKALPAPIEKTISKAKGQPR EPQVCTLPPSRDELTKNQVSLSCAVKGFYPSDIAVEWESNGQPENNYKTTPPVLDSDGSF FLVSKLTVDKSRWQQGNVFSCSVMHIEALHNHYTQKSLSLSPGK, and can be used to form a portion of an immunocytokine composition as described herein. SEQ ID NO: 161 contains the VH of Bevacizumab and hole modifications to the Fc region. Such a sequence can be fused to anti-PD-1 VHH domains described herein (e.g., VHH47), optionally via a peptide linker described herein, to form a building block of an immunocytokine composition.

SEQ ID NO: 162 is QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPREPQ VYTLPPCRDELTKNQVSLWCLVKGFYPSDIAVEWESNGQPENNYKTTPPVLDSDGSFFL YSKLTVDKSRWQQGNVFSCSVMIIHEALHNHYTQKSLSLSPGK, and can be used to form a portion of an immunocytokine composition as described herein. SEQ ID NO: 162 contains the VH of Pembrolizumab and knob modifications to the Fc region. Such a sequence can be fused to anti-VEGFA VHH domains described herein (e.g., VHH175, VHH176, etc.), optionally via a peptide linker described herein, to form a building block of an immunocytokine composition.

SEQ ID NO: 165 is QVQLVQSGVEVKKPGASVKVSCKASGYTFTNYYMYWVRQAPGQGLEWMGGINPSNG GTNFNEKFKNRVTLTTDSSTTTAYMELKSLQFDDTAVYYCARRDYRFDMGFDYWGQG TTVTVSSASTKGPSVFPLAPSSKSTSGGTAALGCLVKDYFPEPVTVSWNSGALTSGVHTF PAVLQSSGLYSLSSVVTVPSSSLGTQTYICNVNHKPSNTKVDKKVEPKSCDKTHTCPPCP APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAKGQPREPQ VYTLPPCRDELTKNQVSLWCLVKGFYPSDIAVEWESNGQPENNYKTTPPVLDSDGSFFL YSKLTVDKSRWQQGNVFSCSVMHEALHNHYTQKSLSLSPGKGGGGSGGGGSGGGGSG GGGSDVQLVESGGGLVQPGGSLRLSCAASGRTFSSYSMGWFRQAPGKEREFVVAISKG GYKYDAVSLEGRFTISRDNAKNTVYLQINSLRPEDTAVYYCASSRAYGSSRLRLADTYE YWGQGTLVTVSS, and can be used to form a portion of an immunocytokine composition as described herein. SEQ ID NO: 165 contains the VH of Pembrolizumab, knob modifications to the Fc region, and anti-VEGFA VHH 175. Such a sequence can be coupled with a hole-modified Fc domain containing sequence, such as one containing an IL-2 polypeptide as a building block of an immunocytokine composition.

### Exemplary Conjugate Immunocytokines

Non-limiting examples of immunocytokine compositions according to the instant disclosure are provided in the table below. For each immunocytokine, one IL-2 polypeptide is conjugated to the VEGF/PD1 binding scaffold at residue K248 of the Fc region (EU numbering) via AJICAP technology and the bifunctional linking reagent

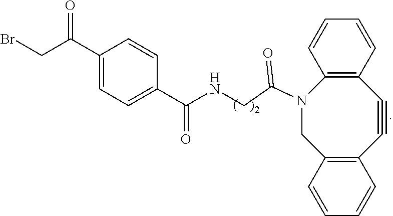

TABLE 23
Exemplary Conjugated Immunocytokine Compositions

| | VEGF/PD1 | | | |
|---|---|---|---|---|
| | Binding | Light | | |
| | Scaffold | Chain | | |
| Composition | Domain | SEQ ID | Heavy Chain | IL-2 |
| Number | Architecture | NO | SEQ ID NO | Polypeptide |
| 217 | Format of | 126 | SEQ ID NO: 170; | 2P72 |
| | FIG. 2A | | GK; SEQ ID NO: | |
| | | | 30; [Reference | |
| | | | anti-PD-1 | |
| | | | ScFv A] | |
| 219 | Format of | 126 | SEQ ID NO: 170; | 2P72 |
| | FIG. 2C | | GK; SEQ ID NO: | |
| | | | 30; [Reference | |
| | | | anti-PD-1 | |
| | | | VHH A] | |
| 218 | Format of | 126 | SEQ ID NO: 170; | 2P73 |
| | FIG. 2A | | GK; SEQ ID NO: | |
| | | | 30; [Reference | |
| | | | anti-PD-1 | |
| | | | ScFv A] | |
| 220 | Format of | 126 | SEQ ID NO: 170; | 2P73 |
| | FIG. 2C | | GK; SEQ ID NO: | |
| | | | 30; [Reference | |
| | | | anti-PD-1 | |
| | | | VHH A] | |
| 221 | Format of | 47 | 258 | 2P72 |
| | FIG. 2D | | | |
| 222 | Format of | 47 | 258 | 2P73 |
| | FIG. 2D | | | |
| 230 | Anti-PD-1 | 47 | 46 | 2P80 |
| | antibody, | | | |
| | no VEGF | | | |
| | binding | | | |
| | domain | | | |
| 223 | Format of | 126 | SEQ ID NO: 170; | 2P80 |
| | FIG. 2C | | GK; SEQ ID NO: | |
| | | | 30; [Reference | |
| | | | anti-PD-1 | |
| | | | VHH A] | |
| 224 | Anti-PD-1 | 47 | 46 | 2P74 |
| | antibody, | | | |
| | no VEGF | | | |
| | binding | | | |
| | domain | | | |
| 225 | Anti-PD-1 | 47 | 46 | 2P75 |
| | antibody, | | | |
| | no VEGF | | | |
| | binding | | | |
| | domain | | | |
| 226 | Anti-PD-1 | 47 | 46 | 2P76 |
| | antibody, | | | |
| | no VEGF | | | |
| | binding | | | |
| | domain | | | |
| 227 | Anti-PD-1 | 47 | 46 | 2P77 |
| | antibody, | | | |
| | no VEGF | | | |
| | binding | | | |
| | domain | | | |
| 228 | Anti-PD-1 | 47 | 46 | 2P78 |
| | antibody, | | | |
| | no VEGF | | | |
| | binding | | | |
| | domain | | | |
| 229 | Anti-PD-1 | 47 | 46 | 2P79 |
| | antibody, | | | |
| | no VEGF | | | |
| | binding | | | |
| | domain | | | |

SEQ ID NO: 170 is EVQLVESGGGLVQPGGSLRLSCAASGYTFTNYGMNWVRQAPGKGLEWVGWINTYTGE PTYAADFKRRFTFSLDTSKSTAYLQMNSLRAEDTAVYYCAKYPHYYGSSHWYFDVWG QGTLVTVSSASTKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKVEPKSCDKTHTCP PCPAPEAAGGPSVFLFPPKPKDTLMISRTPEVTCVVVDVSHEDPEVKFNWYVDGVEVHN AKTKPREEQYNSTYRVVSVLTVLHQDWLNGKEYKCKVSNKALPAPIEKTISKAKGQPR EPQVYTLPPSRDELTKNQVSLTCLVKGFYPSDIAVEWESNGQPENNYKTTPPVLDSDGSF FLYSKLTVDKSRWQQGNVFSCSVMHEALHNHYTQKSLSLSP.

### Domain Architecture of Immunocytokine Compositions

In the following section, the “first binding domain” refers to the anti-PD-1 binding domain of an immunocytokine composition and the “second binding domain” refers to the anti-VEGFA binding domain of the immunocytokine composition. Such binding domains can be any of the binding domains described herein.

In some embodiments, an immunocytokine composition comprises a structure of the formula:

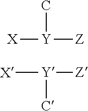

wherein:
Y is the first CH2 and CH3 domains of the Fc domain;
Y′ is the second CH2 and CH3 domains of the Fc domain;
   X and X′ are each independently the first binding domain, the second binding domain, a copy of the first binding domain, a copy of the second binding domain, the cytokine, a copy of the cytokine, a masking polypeptide for the cytokine, or absent;
   Z and Z′ are each independently the first binding domain, the second binding domain, a copy of the first binding domain, a copy of the second binding domain, the cytokine, a copy of the cytokine, a third binding domain targeting PD-1, or a fourth binding domain targeting VEGFA, a masking polypeptide for the cytokine, or absent;
   C and C′ are each independently the cytokine, a copy of the cytokine, or absent, wherein C and C′, if present, are attached to a side chain of a residue of the Fc domain via a linker;
   wherein X, Y, and Z and X′, Y′ and Z′ are depicted in an N-terminal to C-terminal direction; and
   wherein each of X, X′, Z, and Z′ are independently and optionally connected to Y or Y′ via a peptide linker.

In some embodiments, X is the first binding domain; X′ is a copy of the first binding domain, the cytokine, or absent; one of Z or Z′ is the second binding domain and the other is absent, the cytokine, or a copy of the second binding domain; and one of C or C′ is the cytokine and the other is absent, or both C and C′ are absent. In some embodiments, X′ is the cytokine and C and C′ are both absent. In some embodiments, X′ is the copy of the first binding domain and one of C or C′ is the cytokine. In some embodiments, X′ is the copy of the first binding domain, one of Z or Z′ is the second binding domain and the other is the cytokine, and both C and C′ are absent. In some embodiments, Z is the second binding domain and Z′ is a copy of the second binding domain; Z is the second binding domain and Z′ is absent; or Z is absent and Z′ is the second binding domain.

In some embodiments, X is the second binding domain, X′ is a copy of the second binding domain, the cytokine, or absent; one of Z or Z′ is the first binding domain and the other is absent, the cytokine, or a copy of the first binding domain; and one of C or C′ is the cytokine and the other is absent, or both C and C′ are absent. In some embodiments, X′ is the cytokine and C and C′ are both absent. In some embodiments, X′ is a copy of the second binding domain one of C or C′ is the cytokine. In some embodiments, X′ is the copy of the second binding domain, one of Z or Z′ is the second binding domain and the other is the cytokine, and both C and C′ are absent. In some embodiments, Z is the first binding domain and Z′ is a copy of the first binding domain; Z is the first binding domain and Z′ is absent; or Z is absent and Z′ is the first binding domain.

In some embodiments, X is the first binding domain, X′ is the second binding domain, one of C or C′ is the cytokine and the other is absent, Z is absent or the third binding domain, and Z′ is absent or the fourth binding domain. In some embodiments, Z is the third binding domain and Z′ is absent; Z is absent and Z′ is the fourth binding domain; or both Z and Z′ are absent.

In some embodiments, X is the first binding domain, X′ is the second binding domain, one of Z or Z′ is the cytokine and the other is the masking polypeptide for the cytokine, and both C and C′ are absent. In some embodiments, the masking polypeptide for the cytokine is attached to the Y or Y′ to which it is connected via a cleavable peptide linker. In some embodiments, if the third binding domain is present, the first binding domain comprises a Fab and the third binding domain comprises an scFv, and wherein the Fab and the scFv comprise the same VH and VL. In some embodiments, if the fourth binding domain is present, the second binding domain comprises a Fab and the fourth binding domain comprises an scFv, and wherein the Fab and the scFv comprise the same VH and VL.

In some embodiments, X is one of the first or second binding domains and is a Fab, VHH, or scFv. In some embodiments, X is one of the first or second binding domains and is a Fab. In some embodiments, X′ is a copy of X. In some embodiments, one Z or Z′ is one of the first or second binding domains and is an scFv or a VHH, wherein if X is the first binding domain then Z or Z′ is the second binding domain and if X is the second binding domain then Z or Z′ is the first binding domain.

In some embodiments, X is a Fab and the first binding domain, X′ is a copy of the first binding domain, Z is an scFv or VHH and is the second binding domain, Z′ is a copy of the second binding domain, C is the cytokine, and C′ is absent.

In some embodiments, X is a Fab and the second binding domain, X′ is a copy of the second binding domain, Z is an scFv or VHH and is the first binding domain, Z′ is a copy of the first binding domain, C is the cytokine, and C′ is absent.

In some embodiments, X is a Fab and is the first binding domain, X′ is a Fab and is the second binding domain, Z and Z′ are absent, and C or C′ is the cytokine and the other is absent.

In some embodiments, X is a Fab and is the first binding domain, X′ is a Fab and is the second binding domain; Z is an scFv or VHH and is the third binding domain, Z′ is absent, and C or C′ is the cytokine and the other is absent.

In some embodiments, X is a Fab and is the second binding domain, X′ is a Fab and is the first binding domain, Z is an scFv or VHH and is the fourth binding domain, Z′ is absent, and C or C′ is the cytokine and the other is absent.

In some embodiments, X is a Fab and is the first binding domain, X′ is the cytokine, Z is an scFv or VHH and is the second binding domain, Z′ is a copy of the second binding domain, and C and C′ are both absent.

In some embodiments, X is a Fab and is the second binding domain, X′ is the cytokine, Z is an scFv or VHH and is the first binding domain, Z′ is a copy of the first binding domain, and C and C′ are both absent.

In some embodiments, X is a Fab and is the first binding domain, X′ is an scFv and is the second binding domain; Z and Z′ are both absent, and C or C′ is the cytokine and the other is absent.

In some embodiments, X is a Fab and is the second binding domain, X′ is an scFv and is the first binding domain, Z and Z′ are both absent, and C or C′ is the cytokine and the other is absent.

In some embodiments, X is a Fab and is the first binding domain, X′ is Fab or ScFv and is the second binding domain, one of Z or Z′ is the cytokine and the other is the masking polypeptide for the cytokine, and C and C′ are both absent.

In some embodiments, X is a Fab and is the second binding domain, X′ is a Fab or scFv and is the first binding domain, one of Z or Z′ is the cytokine and the other is the masking polypeptide for the cytokine, and C and C′ are both absent.

In some embodiments, only one cytokine is present in the immunocytokine composition.

In some embodiments wherein X, X′, Z, and/or Z′ are one of the binding domains, it is expressly contemplated that the binding domain can be linked to another binding domain in addition to the Fc domain. For example, in cases where Z is a VHH, the VHH of Z can be further linked to another binding domain (e.g., another VHH), either directly or, in preferred instances, by a peptide linker. For example, in some embodiments, Z is a VHH linked to another (i.e., a second) VHH via a peptide linker. An example of such a multifunctional immunocytokine composition is depicted in FIG. 8. In preferred embodiments, the two VHHs will share the same CDR sequences. In some embodiments, the two VHHs will comprise an identical amino acid sequence. In some embodiments, the second VHH will contain additional amino acids added to the C-terminus of the VHH (e.g., a series of one or more prolines attached to the C-terminus compared to the first VHH). In some embodiments, the two VHHs will have an identical amino acid sequence. In some embodiments wherein X, X′, Z, and/or Z′ are one of the binding domains, the binding domain is not further linked to an additional binding domain (i.e., the binding domain of X, X′, Z, and/or Z′ is the only binding domain at the position).

### Pharmaceutical Compositions

In one aspect, provided herein is a pharmaceutical composition comprising an immunocytokine composition described herein; and a pharmaceutically acceptable carrier or excipient. In some embodiments, the pharmaceutical composition further comprises one or more excipients, wherein the one or more excipients include, but are not limited to, a carbohydrate, an inorganic salt, an antioxidant, a surfactant, a buffer, or any combination thereof. In some embodiments the pharmaceutical composition further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical composition further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical composition further comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical composition further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical composition further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical composition is formulated for parenteral or enteral administration. In some embodiments, the pharmaceutical composition is formulated for intravenous (IV) or subcutaneous (SQ) administration. In some embodiments, the pharmaceutical composition is in a lyophilized form.

### Dosage Forms

The immunocytokine compositions described herein can be in a variety of dosage forms. In some embodiments, the immunocytokine composition is dosed as a solution. In some embodiments, the immunocytokine composition is dosed as an injectable solution. In some embodiments, the immunocytokine composition is dosed as an IV solution.

### Methods of Treatment

In one aspect, described herein, is a method of treating cancer in a human subject in need thereof, comprising: administering to the subject an effective amount of an immunocytokine composition described herein or a pharmaceutical composition as described herein.

In some embodiments, the cancer is a solid cancer. A cancer or tumor can be, for example, a primary cancer or tumor or a metastatic cancer or tumor. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

Combination therapies with one or more additional active agents are contemplated herein.

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further treatment of indications is described in more detail elsewhere herein. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the immunocytokine composition is administered in a single dose of the effective amount of immunocytokine composition, including further embodiments in which (i) the immunocytokine composition is administered once a day. In some embodiments, the immunocytokine composition is administered periodically (e.g., multiple doses are administered at different time points).

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “Fc region” or “Fc domain” is used to define the amino acids of a C-terminal region of an immunoglobulin heavy chain. The “Fc region” or “Fc domain” may be a native sequence Fc region or a variant Fc region. As used herein, “Fc region” or “Fc domain” will frequently refer to such a region by itself (e.g., reference to an “Fc region” or “Fc domain” will refer to such amino acids without requiring the presence of any other portion of an antibody) and/or attached to binding domains described herein. Although the boundaries of the Fc region or Fc domain of an immunoglobulin heavy chain might vary, the human IgG heavy chain Fc region is generally defined to stretch from an amino acid residue at position Cys226, or from Pro230, to the carboxyl-terminus thereof (e.g., to K447 (EU numbering)). The numbering of the residues in the Fc region is that of the EU index as in Kabat. The Fc region of an immunoglobulin generally comprises two constant domains, C_{H}2 and C_{H}3. In certain instances herein, an “Fc region” or “Fc domain” as used herein can also stretch from position Ser239 (EU numbering) or from Ala231 (EU numbering).

The term “hinge region” of an antibody or antigen binding fragment herein refers to the flexible part of an antibody molecule that connects a Fab to the Fc region of an antibody (e.g., as in a full-length antibody). In some embodiments, a “hinge region” can be used as a peptide linker to fuse any of the binding domains described herein to an Fc domain (e.g., a hinge region can be used to fuse an scFv binding domain to the N-terminal end of an Fc domain). The “hinge region” of an antibody generally refers residues Glu216 to Pro230 or Glu216 to Pro238 (EU numbering). The “hinge region” can be divided into an “upper portion” corresponding to residues Glu216 to Thr225 (IgG1) or Pro225(IgG4), a “core portion” corresponding to residues Cys226 to Pro230, and a “lower portion” corresponding to residues Ala231 to Pro238. In some instances, the “lower portion” and/or “core portion” of the hinge region can also be considered part of the Fc domain (i.e., the hinge region and the Fc domain can be co-extensive over certain residues). Thus, when a “hinge region” of the instant disclosure is described as a linker between an Fc domain and a binding domain herein, it is expressly contemplated that such a hinge region can include residues which overlap with the Fc domain.

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“V_{H}”) followed by a number of constant domains (“C_{H}”). Each light chain has a variable domain at one end (“V_{L}”) and a constant domain (“C_{L}”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

“Antibodies” from which binding domains of the instant disclosure can be derived encompass, but are not limited to, monoclonal antibodies, polyclonal antibodies, chimeric antibodies, bispecific antibodies, grafted antibodies, multispecific antibodies, heteroconjugate antibodies, humanized antibodies, human antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antigen binding fragment described herein derived from an antibody used as a binding domain as described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™ is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (Kabat et al., Sequences of Proteins of Immunological Interest, 5th Ed., 1991, National Institutes of Health, Bethesda Md., pages 647-669; hereafter “Kabat”); and (2) an approach based on crystallographic studies of antigen-antibody complexes (A1-Iazikani et al. (1997) J. Molec. Biol. 273:927-948)). As used herein, a CDR may refer to CDRs defined by either approach or by a combination of both approaches. In some embodiments, a binding domain includes such a variable region derived from an antibody, or a derivative thereof.

With respect to antibodies and/or binding domains which are derived from antibodies, the term “variable domain” refers to the variable domains of antibodies that are used in the binding and specificity of an antibody for its particular antigen (or such a domain separated from the antibody as a binding domain as provided herein). However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a j-sheet configuration interspersed with three CDRs which form loops connecting and, in some cases, part of the j-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody or a binding domain derived from an antibody (i.e., in a binding domain as described herein) which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat et al., Id. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat et al., Id. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, a “Fab” refers to an antigen binding fragment which comprises one heavy chain variable domain (VH) linked to one constant domain (e.g., a CH1 domain) derived from an antibody and one light chain variable domain (VL) linked to another constant domain (e.g., a light chain constant domain) derived from an antibody. In some embodiments, the VH is linked to a CH1 domain (e.g., is fused to the CH1 domain). In some embodiments, the VL is linked to the light chain constant domain. However, in other embodiments, alternative formats can be used, such as one in which the VH is linked to the light chain constant domain and the VL is linked to a CH1 domain. Such alternative formats can be useful when such Fabs are intended to be used in bispecific antibody formats where light chain pairing can become complicated owing to the present of Fabs which bind to different antigens. Examples of technology where such approaches are used are described by, for example, Klein et al., “The use of CrossMAb technology for the generation of bi- and multispecific antibodies.” Mabs. 2016 August-September; 8(6): 1010-1020.

A CH1 domain according to the instant disclosure (e.g., a CH1 domain of a Fab as provided herein) can be one of those of any of the antibodies described herein, or a variant thereof. For example, a CH1 domain can be one which has at least 80%, 85%, 90%, 95%, 96%, 97%, 98%, 99%, or 100% identity to any one of the following sequences: a) ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKRV (SEQ ID NO: 272); b) ASTKGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRV (SEQ ID NO: 273); c) ASKYGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRV (SEQ ID NO: 274); or d) ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKV (SEQ ID NO: 275).

A light chain constant region according to the instant disclosure can that of a lambda or kappa light chain, or a variant thereof. For example, a light chain constant region can be one which has at least 80%, 85%, 90%, 95%, 96%, 97%, 98%, 99%, or 100% identity to the sequence

(SEQ ID NO: 276)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC,
(SEQ ID NO: 277)
RTVAAPSVFIFPPSDEQLKSGTASVVCLLNNFYPREAKVQWKVDNAKQSG
NSQESVTEQDSKDSTYSLSSTLTLSKADYEKHKVYACEVTHQGLSSPVTK
SFNRGEC,
or
(SEQ ID NO: 278)
RTVAAPSVFIFPPSDEQLKSGTASVVCLLNNFYPREAKVQWKVDNAKQSG
NSQESVTEQDSKDSTYSLSSTLTLSKADYEKHKVYACEVTHQGKSSPVTK
SFNRGEC.

As used herein, an “scFv” refers to a single chain variable fragment which comprises a VH and VL linked via a peptide linker. In some embodiments, the linker is a flexible peptide linker, such as the GS linkers described herein.

As used herein, a “VHH” refers to a single-domain antibody that binds to an antigen. Such single-domain antibodies are also referred to as “nanobodies” in the art. Such VHHs can be derived from the heavy-chain antibodies of camelids. A VHH lacks a corresponding VL domain.

Referred to herein are groups which are “attached” or “covalently attached” to residues of IL-2 polypeptides or other polypeptides. As used herein, “attached” or “covalently attached” means that the group is tethered to the indicated reside, and such tethering can include a linking group (i.e., a linker). Thus, for a group “attached” or “covalently attached” to a residue, it is expressly contemplated that such linking groups are also encompassed.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the federal or a state government or listed in the U.S. Pharmacopeia (U.S.P.) or other generally recognized pharmacopeia for use in animals, including humans.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Throughout the instant description, certain numerical or other similar values may be described as, for example, “at least” or “at most” a set of values indicated in a list form (e.g., “at least 2, 3, 4, 5, or 6”). In such cases, unless context clearly indicates otherwise, it is intended that the phrase “at least,” “at most,” or other similar term is applied individually to each value in the list. For example, the phrase “at least 2, 3, 4, 5, or 6” is to be interpreted as “at least 2, at least 3, at least 4, at least 5, or at least 6.”

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer

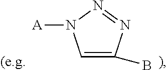

it is intended that the other regioisomer

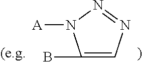

is also encompassed.

The term “subject” refers to an animal which is the object of treatment, observation, or experiment. Byway of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

TABLE 24
Table of Conjugation Handles

| Conjugation | Complementary | Reaction |
|---|---|---|
| Handle | Conjugation Handle | Product |
| Sulfhydryl | alpha-halo-carbonyl | thioether |
| | (e.g., bromoacetamide), | |
| | alpha-beta unsaturated | |
| | carbonyl (e.g., | |
| | maleimide, acrylamide) | |
| Azide | alkyne (e.g., terminal | triazole |
| | alkyne, substituted | |
| | cyclooctyne (e.g., | |
| | dibenzocycloocytne (DBCO), | |
| | difluorocyclooctyne, | |
| | bicyclo[6.1.0]nonyne, | |
| | etc.)) | |
| Phosphine | Azide/ester pair | amide |
| Tetrazine | trans-cyoclooctene | dihydropyrida |
| | | zine |
| Amine | Activated ester (e.g., | amide |
| | N-hydroxysuccinimide ester, | |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine | amide |
| trifluoroborate | (e.g., O-carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methyl ethyl (i-propyl), n-butyl, i-butyl, 5-butyl, n-pentyl, 1,1-dimethyl ethyl (i-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CFF—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C≡C—R^{X}, wherein R^{X} refers to the remaining portions of the alkynyl group. In some embodiments, R^{X} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH3, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —O CH₂CH₂ O CH₂CH₂ O CH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C 1-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an Fc region (e.g., an IgG1 Fc region) (EU numbering). In some embodiments, the desired functionalization is incorporated at residue position 248 of an Fc region (EU numbering). In some embodiments, position 248 corresponds to the 18^{th} residue in a human IgG CH2 region (EU numbering).

In the instant disclosure, SEQ ID NOs: 31, 120, and 701 define sequences of human proteins. The remaining sequences are all synthetic constructs.

Although the present disclosure and its advantages have been described in detail, it should be understood that various changes, substitutions and alterations can be made herein without departing from the spirit and scope of the disclosure as defined in the appended claims

The present disclosure is further illustrated in the following Examples which are given for illustration purposes only and are not intended to limit the disclosure in any way.

### EXAMPLES

### Example 1A—Preparation and Purification

Recombinant IL-2 variants and Fc containing scaffolds can be prepare recombinantly according to methods well understood in the art. Generation of IL-2 polypeptides, conjugation of IL-2 polypeptides, and generation of activatable IL-2 polypeptides has been described previously in, for example, WO2023281485A1 (corresponding US Pat. Pub. No. US20230303649A1), WO2024150175A1, and U.S. Ser. No. 11/633,488B2.

### Example 1B—Preparation and Purification of Compositions Provided Herein

Expression—Recombinant compositions described herein were prepared according to the following general protocol. The recombinant compositions were expressed using ExpiCHO-S cell line (ThermoFisher Scientific) and BalanCD Transfectory CHO (Fujifilm) as culture media. On the day of the transfection, the cells were centrifugated at low speed (500 g/15 min), the media was carefully removed, and the cells were resuspended at a density of 6.0 million cells per ml with fresh BalanCD Transfectory CHO media supplemented with 4 mM L-Glutamine (Gibco). The cells were transfected using FectoPro transfection reagent (Sartorius) as described by the manufacturer. Briefly, for a 1L transfection reaction, a total of 0.8 mg of plasmid DNA mix containing three plasmids encoding the different chains was diluted into 100 ml of non-supplemented BalanCD Transfectory CHO media. The mixture was then poured into a container with 1.6 ml of the FectoPro reagent, mixed vigorously and incubated for 10 minutes at room temperature before the solution was added to the cells. After 4 h of incubation at 37° C. and 8% CO2, the cells were incubated at 32° C. and 5% CO2. At Day 4, 6, 8 and 11, the cells were supplemented with balanCD Feed 4 (Fujifilm) and Glucose (Gibco) to maintain high viability before harvesting on Day 12.

Purification—After expression, the culture media was centrifugated at 2000 g during 15 min to remove cells. The clarified media was then filtered on 0.22 um filter with Diatomaceous earth as filter aid. The molecules were purified on an AKTA Pure coupled with a HiTrap MabSelect Sure column (Cytiva). After elution with 0.1M of glycine pH3.0, the fractions were pooled and neutralized by addition of 10% of the pool volume of 1M acetate pH5.5 and the molecules were polished using a HiTrap SP HP with 50 mM acetate Ph5.0 as mobile phase. The peak corresponding to the molecule of interest was collected, sterile filtered and the purity was assessed using SEC-HPLC, SDS-PAGE and LC-MS analysis.

### Example 1C—Biophysical Characterization of Immunocytokine Compositions

The purity and identity of the recombinant proteins (including those available from commercial sources) and conjugated proteins of compositions described herein is confirmed by aSEC, LC-MS, and other methods according to the following protocols.

Analytical Size Exclusion chromatography (aSEC)—Sample preparation: Samples are diluted to a concentration between 0.5 and 1.0 mg/mL with their respective formulation buffer before centrifugation at 20000×g, 4° C. for 10 min. Material: 10×PBS pH7.2 (Phosphate-Buffered Saline, Ref 70013-032, Gibco). Column: XBridge Premier Protein SEC Column (7.8×300 mm, 2.5 μm, 250 Å pore size, Ref 186009962, Waters). System Suitability Test (SST): BEH200 SEC Protein Standard mix (Ref 186006518, Waters). Flow rate: 0.75 mL/min. Elution buffer: 1×PBS pH 7.2 (Filtered on 0.22 um and sonicated). Temperature: 25° C. Sample loading: 10 uL. Elution: 100% of 1×PBS pH7.2. Data acquisition: 24 min. Wavelengths: 220 nm and 280 nm. Representative retention time and purity data for selected compositions is provided in the table below. aSEC traces showed substantially only a single peak with good peak shape.

TABLE 25
Elution Profile

| | Rt | Purity |
|---|---|---|
| Composition | (min) | (%) |
| 116 | 10.083 | 95.4 |
| 117 | 10.075 | 94.4 |
| 118 | 10.108 | 98.4 |
| 119 | 10.092 | 96.1 |
| 91 | 9.042 | 98.9 |
| 120 | 8.967 | 98.8 |
| 94 | 8.642 | 95.5 |
| 98 | 8.675 | 96.4 |
| 219 | 8.275 | 99.6 |

Hydrophobic chromatography—Sample preparation Samples at 1 mg/mL were spiked with some HIC mobile phase A in order to reach a concentration of 1M of ammonium sulfate before injection (Example: 26.7 uL of protein sample at 1 mg/mL+33.3 uL of mobile phase A) before centrifugation at 20000×g, 4° C. for 10 min. Material: Ammonium sulfate (Emprove Expert, Granulated, MW=132.14 g/mol, Ref 1.04161.1000, Sigma Aldrich); Sodium phosphate monobasic dihydrate (MW=156.01 g/mol, Ref 71505-1KG, Sigma Aldrich); Acetonitrile (for HPLC—Gradient grade, Ref 20060.320, VWR chemicals). Column: TSKgel Butyl-NPR column (4.6×35 m, 2.5 μm, Ref 0014947, TOSOH BIOSCIENCE). References: Ustekinumab, Adalimumab. Flow rate: 0.75 mL/min. Elution buffers: Mobile phase A: 1.8M ammonium sulfate, 0.1M sodium phosphate pH6.5 (Filtered on 0.22 um and sonicated); Mobile phase B: 0.1M sodium phosphate pH6.5 (Filtered on 0.22 um and sonicated) Mobile phase C: 20%/80% v/v Acetonitrile/H2O-MQ (Sonicated). Temperature: 25° C. Sample loading: 10 uL. Elution: Hydrophobic chromatography elution gradient (see table below). Data acquisition: 45 min. Wavelengths: 220 nm and 280 nm.

Samples at 1 mg/mL were mixed with HIC mobile phase A containing 1.8M ammonium sulfate, 0.1M sodium phosphate pH6.5 in order to reach a concentration of 1M of ammonium sulfate and promote hydrophobic interactions (Example: 26.7 uL of protein sample at 1 mg/mL+33.3 uL of mobile phase A). Samples were centrifuged at 20000×g at 4° C. for 10 min before injection to remove any particles.

Hydrophobic interaction chromatography was performed using TSKgel Butyl-NPR column (4.6×35 m, 2.5 μm, Ref 0014947, TOSOH BIOSCIENCE) equilibrated with 1.8M ammonium sulfate, 0.1M sodium phosphate pH6.5. 10 μL of sample was injected at a flow rate of 0.75 mL/min. Elution was achieved by applying a linear gradient of decreasing salt concentration using a mobile phase B containing 0.1M sodium phosphate pH6.5. Proteins were monitored by UV absorbance at 220 nm. The elution profile is recorded and compared to reference molecules (Ustekinumab, Adalimumab)

TABLE 26
Hydrophobic Interaction Chromatography Elution Times

| | Mobile | Mobile | Mobile |
|---|---|---|---|
| | phase | phase | phase |
| Time (min) | A (%) | B (%) | C (%) |
| 0 | 100 | 0 | 0 |
| 2 | 100 | 0 | 0 |
| 19.1 | 0 | 100 | 0 |
| 22 | 0 | 100 | 0 |
| 22.1 | 0 | 0 | 100 |
| 30 | 0 | 0 | 100 |
| 30.1 | 0 | 100 | 0 |
| 30.2 | 100 | 0 | 0 |
| 45 | 100 | 0 | 0 |

Retention times of selected compositions in the described hydrophobic interaction chromatography method are provided in the table below. Traces showed substantially only a single peak with good peak shape.

TABLE 27
Hydrophobic interaction chromatography retention
times of selected compositions

| | HIC |
|---|---|
| | RT |
| Composition | (min) |
| composition | 13.603 |
| 116 | |
| composition | 13.443 |
| 117 | |
| composition | 13.63 |
| 118 | |
| composition | 13.46 |
| 119 | |
| composition | 16.56 |
| 91 | |
| composition | 16.1 |
| 98 | |

Heparin chromatography—Sample preparation: Samples are diluted to 0.2 mg/mL with some 20 mM Histidine pH5.5 before centrifugation at 20 000×g at 4° C. for 10 min. Column: TSKgel Heparin-5 PW (7.5 mm×7.5 cm, 10 μm, Ref 13064, TOSOH BIOSCIENCE). References: Ustekinumab, Adalimumab, Briakinumab, Gantenerumab, Daratumumab, Obinutuzumab. Flow rate: 0.5 mL/min. Elution buffers: Mobile phase A: 50 mM Tris pH7.4 (Filtered on 0.22 um and sonicated); Mobile phase B: 20 mM Tris pH7.4; 1M NaCl (Filtered on 0.22 um and sonicated). Temperature: 25° C. Sample loading: 50 uL (10 ug). Elution: Heparin chromatography elution gradient (See table). Data acquisition: 45 min. Wavelengths: 220 nm and 280 nm. Results: Due to different HPLC systems, samples were compared by using their relative retention time (Rel. RT). It has been calculated by dividing their retention time (min) by the one of a control, Adalimumab.

TABLE 28
Heparin Chromatography Elution Gradient

| | Mobile | Mobile |
|---|---|---|
| | phase | phase |
| Time (min) | A (%) | B (%) |
| 0 | 100 | 0 |
| 3.2 | 100 | 0 |
| 29.6 | 45 | 55 |
| 30.4 | 0 | 100 |
| 36.8 | 0 | 100 |
| 37.6 | 100 | 0 |
| 45 | 100 | 0 |

The propensity for charge-mediated heparin binding was assessed using Heparin chromatography. A higher affinity for heparin is indicative of increased charge-mediated interactions at the surface of endothelial cells, suggesting a greater risk of non-specific clearance via pinocytosis, which is an important consideration in evaluating molecule developability.

Samples were prepared by diluting to a concentration of 0.2 mg/mL in 20 mM Histidine buffer at pH 5.5. The diluted samples were then centrifuged at 20,000×g at 4° C. for 10 minutes to remove any particulate matter prior to chromatography. 50 μL of sample is injected on a TSKgel Heparin-5 PW column (7.5 mm×7.5 cm, 10 m particles; Ref 13064, TOSOH BIOSCIENCE) equilibrated with 50 mM Tris, pH 7.4 at a flow rate of 0.5 mL/min. Sample is eluted using a gradient of 20 mM Tris, pH 7.4, containing 1 M NaCl. Proteins were monitored by UV absorbance at 280 nm. The elution profile was recorded and compared to reference molecules (Ustekinumab, Briakinumab, Gantenerumab, Daratumumab, and Obinutuzumab). To facilitate comparison of molecules analyzed on different HPLC systems, the relative retention time (Heparin Rel. RT) compared to the reference antibody Adalimumab is provided.

Heparin chromatography retention times and relative retention times compared to Adalimumab for selected compositions is provided in the Table below. Traces showed substantially only a single peak with good peak shape. For reference, in the trace in which Composition 94's retention time was measured to be 14.618, reference antibodies showed the following retention times: Daratumumab—12.1 minutes; Obinutuzumab—12.4 minutes; Ustekinumab—14.8 minutes; Briakinumab—21.6 minutes; and Gantenerumab—24.2 minutes.

TABLE 29
Heparin Chromatography Retention
Times for Selected Compositions

| | Heparin | Heparin |
|---|---|---|
| | RT | Rel. RT |
| Composition | (min) | (min) |
| composition | 18.07 | 1.024 |
| 116 | | |
| composition | 17.988 | 1.019 |
| 117 | | |
| composition | 17.993 | 1.024 |
| 118 | | |
| composition | 17.898 | 1.019 |
| 119 | | |
| composition | 14.597 | 0.831 |
| 91 | | |
| composition | 14.49 | 0.825 |
| 98 | | |
| composition | 14.618 | 0.832 |
| 94 | | |
| composition | 14.49 | 0.825 |
| 120 | | |
| Composition | 23.128 | 1.317 |
| 1 | | |
| Composition | 23.184 | 1.32 |
| 2 | | |
| Composition | 21.11 | 1.202 |
| 3 | | |
| Composition | 22.575 | 1.33 |
| 9 | | |
| Composition | 20.183 | 1.189 |
| 10 | | |
| Composition | 22.955 | 1.352 |
| 14 | | |
| Composition | 23.182 | 1.366 |
| 19 | | |
| Composition | 23.093 | 1.36 |
| 25 | | |
| Composition | 23.158 | 1.364 |
| 26 | | |
| Composition | 21.069 | 1.242 |
| 27 | | |
| Composition | 20.998 | 1.237 |
| 28 | | |
| Composition | 21.21 | 1.249 |
| 29 | | |
| Composition | 21.185 | 1.223 |
| 30 | | |
| Composition | 20.854 | 1.229 |
| 31 | | |
| Composition | 20.717 | 1.196 |
| 32 | | |
| Composition | 23.153 | 1.337 |
| 38 | | |
| Composition | 20.883 | 1.189 |
| 59 | | |
| Composition | 20.993 | 1.196 |
| 60 | | |
| Composition | 21.012 | 1.197 |
| 61 | | |
| Composition | 20.897 | 1.19 |
| 62 | | |
| Composition | 20.997 | 1.196 |
| 63 | | |
| Composition | 20.845 | 1.187 |
| 64 | | |
| Composition | 20.893 | 1.19 |
| 65 | | |
| Composition | 20.852 | 1.188 |
| 66 | | |
| Composition | 21.137 | 1.204 |
| 67 | | |
| Composition | 21.277 | 1.212 |
| 68 | | |
| Composition | 21.307 | 1.214 |
| 69 | | |
| Composition | 21.16 | 1.205 |
| 70 | | |
| Composition | 21.253 | 1.21 |
| 71 | | |
| Composition | 21.082 | 1.201 |
| 72 | | |
| Composition | 21.133 | 1.204 |
| 73 | | |
| Composition | 21.085 | 1.201 |
| 74 | | |
| Composition | 20.607 | 1.174 |
| 75 | | |
| Composition | 20.717 | 1.18 |
| 76 | | |
| Composition | 20.703 | 1.179 |
| 77 | | |
| Composition | 20.605 | 1.174 |
| 78 | | |
| Composition | 20.692 | 1.178 |
| 79 | | |
| Composition | 20.535 | 1.17 |
| 80 | | |
| Composition | 20.637 | 1.175 |
| 81 | | |
| Composition | 20.545 | 1.17 |
| 82 | | |

Mass Spectrometry (Performed by Functional Genomics Center Zurich)—Sample preparation: All samples analysed under non reducing or reducing conditions are diluted with 1% TFA, passed through the AttractFiltra RC Micro Spin column and transferred to an autosampler vials for LC/MS. In case of reducing conditions, samples are reduced with dithiothreitol (DTT) at a final concentration of 50 mM prior to the TFA dilution and the Micro Spin column. The reduction is performed for 1 h at RT and at pH 8. Column: BioResolve Premier RP mAb (450 Å, 2.7 μm, 2.1 mm×20 mm, Waters). System: Synapt G2-Si mass spectrometer coupled to an Acquity UPLC station. Flow rate: 0.2 mL/min. Elution buffers: Buffer A: 0.1% DFA in water; Buffer B: 0.1% DFA in AN/75% 2-PrOH. Temperature: 80° C. Elution: Gradient. Data acquisition: 30 min. Mode: Positive-ion mode. m/z range: From 400 to 5000 Da. Spray voltage: 120V. Source temperature: 100° C. Software: MassLynx 4.2 (Waters). Results are shown for selected compositions in the tables below.

TABLE 30
Non reduced LC-MS measured and
expected molecular weight (Da)

| | | Expected | MW |
|---|---|---|---|
| | Measured | MW | delta |
| Composition | MW (Da) | (Da) | (Da) |
| Composition | 145731 | 142851 | 2880.2 |
| 116 | | | (Glycans) |
| Composition | 146122 | 143239 | 2882.7 |
| 118 | | | (Glycans) |
| Composition | 146138 | 143255 | 2882.8 |
| 119 | | | (Glycans) |
| Composition | 192847 | 189781 | 3065.8 |
| 91 | | | (Glycans) |
| Composition | 193216 | 190146 | 3070.4 |
| 120 | | | (Glycans) |
| Composition | 193598 | 190537 | 3060.9 |
| 219 | | | (Glycans) |
| Composition | 145747 | 142867 | 2880.2 |
| 117 | | | (Glycans) |
| Composition | 192859 | 189764 | 3094.8 |
| 94 | | | (Glycans) |
| Composition | 192876 | 189780 | 3095.9 |
| 98 | | | (Glycans) |

TABLE 31
Reduced LC-MS measured and expected molecular weight (Da)

| | Measured | Expected | Delta | Measured | Expected | Delta | Measured | Expected | Delta |
|---|---|---|---|---|---|---|---|---|---|
| Composition | LC | LC(Da) | LC(Da) | HC1 (Da) | HC1 (Da) | HC1 (Da) | HC2 (Da) | HC2 (Da) | HC2 (Da) |
| Composition 116 | 23739.5 | 23744.3 | −4.8 | 79866.5 | 78449.9 | 1416.6 | 42126 | 40686.9 | 1439.1 |
| Composition 118 | 23739.5 | 23744.3 | −4.8 | 80256 | 78838.3 | 1417.7 | 42128.5 | 40686.9 | 1441.6 |
| Composition 119 | 23739.5 | 23744.3 | −4.8 | 80256 | 78838.3 | 1417.7 | 42142 | 40702.9 | 1439.1 |
| Composition 91 | 23461 | 23465.8 | −4.8 | 78940 | 77505.6 | 1434.4 | 66820.5 | 65384.3 | 1436.2 |
| Composition 120 | 23461 | 23465.8 | −4.8 | 79304.5 | 77870 | 1434.5 | 66820.5 | 65384.3 | 1436.2 |
| Composition 219 | 23446 | 23445.7 | 0.3 | 81719 | 80282.4 | 1436.6 | 64827.5 | 63391.4 | 1436.1 |
| Composition 117 | 23739.5 | 23744.3 | −4.8 | 79867.5 | 78449.9 | 1417.6 | 42142.5 | 40702.9 | 1439.6 |
| Composition 94 | 23461 | 23465.8 | −4.8 | 78942 | 77505.6 | 1436.4 | 66835 | 65397.3 | 1437.7 |
| Composition 98 | 23462 | 23465.8 | −3.8 | 66851.5 | 65413.2 | 1438.3 | 78942 | 77505.6 | 1436.4 |

VEGF cooperativity assessment by aSEC and Dynamic Light Scattering (DLS)—Selected compositions were assessed for the degree of coopreativity of the compositions in the presence of VEGF. In the presence of VEGF, dual binding compositions can form non-covalent multimers of multiple compositions bounds to a VEGF multimer (e.g., VEGF dimers). In some instances, the formation of these multimers may provide enhanced efficacy of the immunocytokine composition in the presence of VEGF due to avidity effects. Here, changes in particle size (as measured by DLS or aSEC) can be indicative of the ability of immunocytokine constructs to form these multimers.

Sample preparation: All samples were prepared without VEGF as well as with 2× of the VEGF dimer (human VEGFA (27-107)-2x(G4S)-8His produced in house or Human VEGF165 Protein, His Tag (Cat. VE5-H5248, Acro Biosystems)) before centrifugation at 20000×g, 4° C. for 15 min.

Analytical Size Exclusion Chromatography for VEGF cooperativity assessment—Column: XBridge BEH200A SEC column (7.8×300 mm, 3.5 μm, 200A pore size, Waters). System Suitability Test (SST): BEH200 SEC Protein Standard mix (Ref 186006518, Waters). Flow rate: 0.86 mL/min. Elution buffer: 50 mM sodium phosphate dibasic dihydrate, 628 mM Sodium chloride, 13.5 mM potassium chloride, 10 mM monobasic potassium phosphate pH7.2. Temperature: 25° C. Sample loading: 10 uL. Elution: 100% of elution buffer. Data acquisition: 24 min. Wavelengths: 220 nm and 280 nm.

Dynamic Light Scattering (DLS) for VEGF cooperativity assessment—System: Prometeus PANTA (NanoTemper) with Standard sensitivity capillaries (Ref PR-C002). Temperature: 25C. DLS power: 100%.

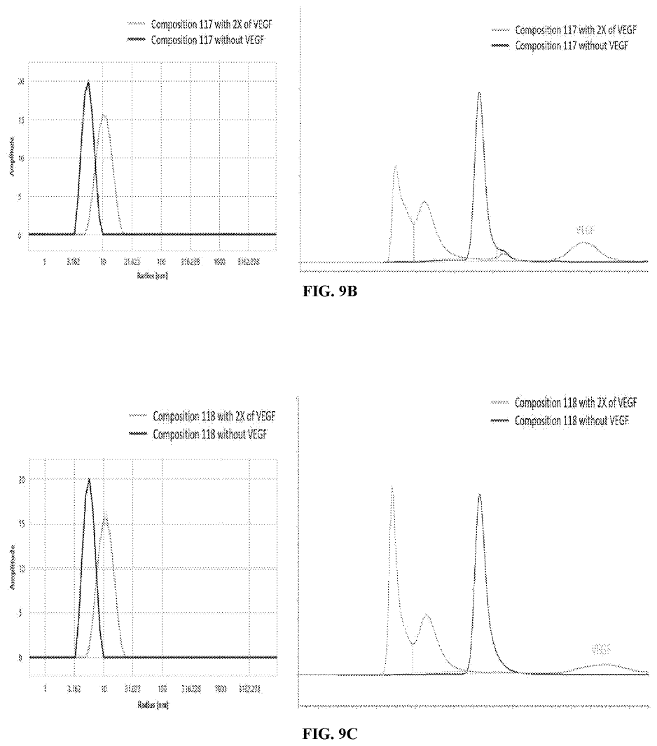

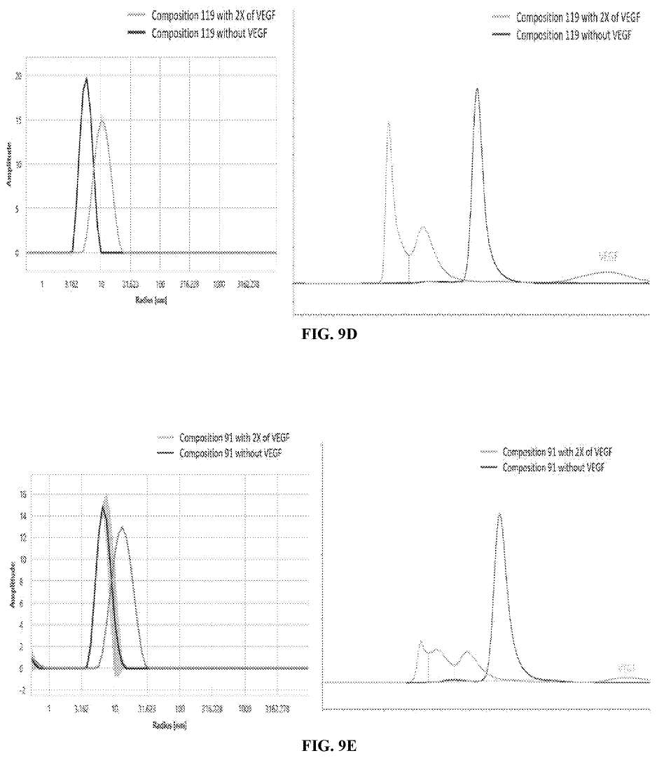

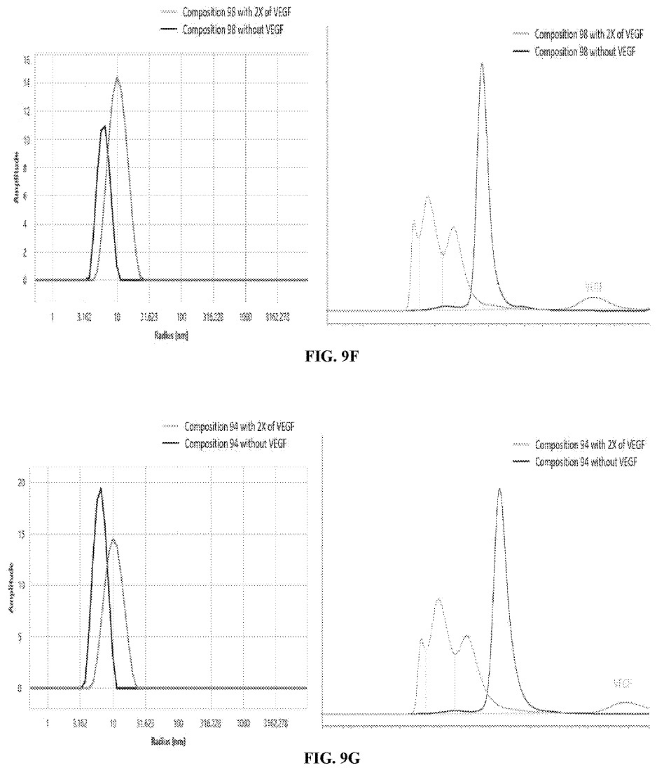

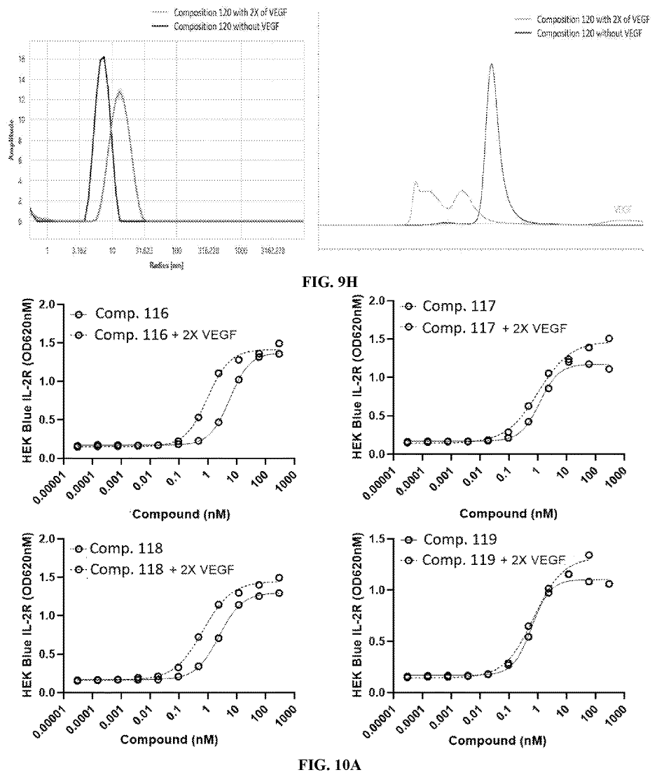

Results of these experiments for selected compositions are shown in FIGS. 9A-9H. Each of FIGS. 9A-9H shows a DLS trace (left panel) and an aSEC trace (right panel) for each indicated composition. FIG. 9A shows results for Composition 116. FIG. 9B shows results for Composition 117. FIG. 9C shows results for Composition 118. FIG. 9D shows results for Composition 119. FIG. 9E shows results for Composition 91. FIG. 9F shows results for Composition 98. FIG. 9G shows results for Composition 94. FIG. 9H shows results for Composition 120. DLS results for the indicated samples are provided in the table below.

TABLE 32
DLS Results for Indicated Compositions +/− VEGF

| | | Polydispersity |
|---|---|---|
| Composition | Radius (nm) | Index (PDI) |
| composition 116 | 5.64 | 0.05 |
| composition 116 with 2X VEGF | 11.07 | 0.09 |
| composition 117 | 5.54 | 0.05 |
| composition 117 with 2X VEGF | 10.54 | 0.09 |
| composition 118 | 5.62 | 0.05 |
| composition 118 with 2X VEGF | 10.78 | 0.09 |
| composition 119 | 5.58 | 0.05 |
| composition 119 with 2X VEGF | 10.48 | 0.09 |
| composition 91 | 6.9 | 0.05 |
| composition 91 with 2X VEGF | 13.14 | 0.12 |
| composition 98 | 6.24 | 0.04 |
| composition 98 with 2X VEGF | 10.35 | 0.12 |
| composition 94 | 6.37 | 0.05 |
| composition 94 with 2X VEGF | 10.31 | 0.1 |
| composition 120 | 7.06 | 0.06 |
| composition 120 with 2X VEGF | 13.24 | 0.12 |

### Example 2—Conjugation of IL-2 Polypeptide to Fc Containing Binding Domain Constructs

Preparation of conjugatable binding domain constructs

An Fc domain of a construct containing the desired binding domain architecture is modified to incorporate a desired conjugation handle (e.g., DBCO) to facilitate coupling of the IL-2 polypeptide (or other cytokine as described herein). In this example, a DBCO conjugation handle is added to residue K248 (EU numbering) of the Fc domain using a protocol modified from Examples 2-4 of US Patent Publication No. US2020019165A1 using AJICAP™ technology. Briefly, the Fc domain containing binding domain construct with a free sulfhydryl group attached to a lysine residue side chain in the Fc region is prepared by reacting the Fc domain containing construct with an affinity peptide configured to deliver a protected version of the sulfhydryl group (e.g., a thioester) to the lysine residue. The protecting group is then removed to reveal the free sulfhydryl. The free sulfhydryl is then reacted with a bifunctional reagent comprising a bromoacetamide group connected to the DBCO conjugation handle through a linking group (e.g.,

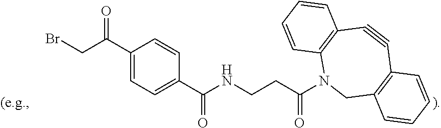 [SMILES: O=C(CBr)c1ccc(C(=O)NCCC(=O)N2Cc3ccccc3C#Cc3ccccc32)cc1]

The method can be used to produce an Fc domain with one DBCO group present (DAR1) and/or two DBCO groups attached to the Fc domain (DAR2, one DBCO group linked to each chain of the Fc domain). In the instant disclosure, it is preferred that DAR1 constructs are prepared (i.e., the constructs contain only a single cytokine attached). In some instances in which an asymmetric anti-VEGFA/PD-1 binding domain containing construct is used, the residue at the desired point of attachment can be mutated to ensure that a DAR1 construct is prepared (e.g., one of the polypeptide chains of the Fc domain contains a mutation of the K248 residue, such as a K248A substitution).

### Conjugation of Fc Domain Construct to IL-2 Polypeptide

The DBCO modified Fc domain of the binding domain construct (i.e., the anti-VEGFA/PD-1 construct) is then conjugated to an IL-2 polypeptide comprising an azide moiety at a desired point of attachment (e.g., an IL-2 polypeptide which contains an amino acid with an azide side chain or an IL-2 linked to an azide). DBCO modified Fc domain containing construct is reacted with 2-10 equivalents of azide containing IL-2 (pH 5.2 buffer, 5% trehalose, rt, 24 h) (e.g., 1-10 equivalents of the IL-2 polypeptide).

### Purification and Characterization of Multifunctional Immunocytokine

The resulting immunocytokine is purified by cation-exchange chromatography and/or size exclusion chromatography to obtain purified immunocytokine. Multifunctional immunocytokine is purified from unreacted IL-2 and aggregates using a desalting column, CIEX and SEC (GE Healthcare Life Sciences AKTA pure, mobile phase: Histidine 5.2/150 mM NaCl/5% Trehalose, column: GE Healthcare Life Sciences SUPERDEX™ 200 increase 3.2/300, flow rate: 0.5 mL/min).

The purity and identity of the IL-2 polypeptide containing multifunctional immunocytokine is confirmed by RP-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN:IPA:ETOH:H2O (5:1.5:2:1.5), flow rate: 0.5 mL/min, injection amount: 10 g (10 μL Injection of 1 mg/mL), gradient: 0% to 20% mobile phase B in 50 min) and SDS-PAGE.

Compositions 217-230 with conjugation to IL-2 polypeptides according to the instant disclosure are manufactured according to analogous methods to those described above. For each of these compositions, a single IL-2 polypeptide is included in the composition.

### Example 3—Characterization of Immunocytokine IL-2 Activity

The ability of the immunocytokine to perform various IL-2 activities is measured as provided below, as well as relevant comparisons to non-conjugated IL-2 polypeptides. Description of suitable assays and other tests which can be performed to assess the IL-2 activity of the immunocytokine compositions can be found, for example, in WO2024150175A1 (in particular for assessment of activatable or masked IL-2 polypeptides), WO2023281479A1 (in particular for tests of the IL-2 activity of IL-2/anti-PD-1 immunocytokines), and WO2023281479A1.

### Example 3A—HEK Blue IL2 Receptor (CD25+/CD122+/CD132+) Reporter Assay

In order to assess the ability of immunocytokines to signal via the IL-2 moiety, a HEK Blue reporter assay was performed in CD25+/CD122+/CD132+ cells. Briefly, an IL-2R αβγ positive HEK-Blue reporter cell line was used to determine binding of IL-2 immunocytokines (and activated versions thereof) to IL-2Rαβγ and subsequent downstream signaling that resulted in activation of a STAT5 transcriptional reporter. The general protocol was as follows: 5×104 cells HEK-Blue™ IL-2R αβγ reporter cells (InvivoGen, #hkb-il2-2) were seeded into each well of a 96 well plate and stimulated with 0-100 nM of IL-2 immunocytokine variants in the presence of recombinant human VEGF165 (VE5-H5248, Acro Bio) in a ratio 2:1 to the test article at 37° C. and 5% CO2. After a 20 h incubation, 20 μL of cell culture supernatant was then taken from each well and mixed with 180 μL QUANTI-Blue™ media in a 96 well plate, then incubated for 1 hour at 37° C. and 5% CO2 (with 600 rpm shaking). The absorbance signal at 630 nm was then measured on an Enspire® plate reader. Half Maximal Effective dose (EC50) was calculated based on a variable slope, four parameter analysis using GraphPad PRISM® software. Results for selected immunocytokine compositions are provided in the Tables below.

TABLE 33
IL2 Payload Screen via HEKBlue ™ Assay

| Composition Number | Average EC50 (nM) |
|---|---|
| Composition 6 | 0.00074 |
| Composition 7 | 2.607 |
| Composition 8 | 2.096 |
| Composition 9 - batch 1 | 0.7738 |
| Composition 9 - batch 2 | 0.8394 |
| Composition 10 - batch 1 | 21.87 |
| Composition 10 - batch 2 | 113.4 |
| Composition 11 | 0.09121 |
| Composition 12 - batch 1 | 0.01296 |
| Composition 12 - batch 2 | 0.01578 |
| Composition 13 | 0.3769 |
| Composition 14 - batch 1 | 106.2 |
| Composition 14 - batch 2 | 145 |
| Composition 15 | 0.1324 |
| Composition 16 | 0.1298 |
| Composition 17 | 6.035 |
| Composition 18 | 0.2832 |
| Composition 19 - batch 1 | 19.06 |
| Composition 19 - batch 2 | 16.62 |
| Composition 20 | 1.44 |
| Composition 21 | 0.1453 |
| Composition 22 | 0.03497 |
| Composition 23 | 36.72 |
| Composition 24 | 0.2134 |
| Composition 25 | 180 |
| Composition 25 | 184.55 |
| Composition 26 - batch 2 | 10.225 |
| Composition 27 | NA |
| Composition 28 - batch 3 | 22.725 |
| Composition 29 - batch 2 | 0.4717 |
| Composition 31 - batch 2 | NA |
| Composition 32 - batch 2 | 73.34 |
| Composition 33 | 1.526 |
| Composition 34 | 5.591 |
| Composition 35 - batch 2 | 9.51 |
| Composition 35 - batch 1 | 0.04626 |
| Composition 36 | 0.00688 |
| Composition 37 | 0.07064 |
| Composition 38 | 0.02176 |
| Composition 39 | 0.02096 |
| Composition 40 - batch 1 | 4.765 |
| Composition 40 - batch 2 | 5.4275 |
| Composition 41 - batch 1 | 0.6426 |
| Composition 41 - batch 2 | 1.24135 |
| Composition 42 | NA |
| Composition 43 | 0.5178 |
| Composition 44 - batch 1 | 9.808 |
| Composition 44 - batch 2 | 31.16 |
| Composition 45 | 0.5856 |
| Composition 46 - batch 1 | 1.387 |
| Composition 46 - batch 2 | 1.2394 |
| Composition 47 | 0.04507 |
| Composition 48 - batch 1 | 2.361 |
| Composition 48 - batch 2 | 16.24 |
| Composition 26 - batch 1 | 5.332 |
| Composition 27 | NA |
| Composition 28 - batch 1 | 5.75 |
| Composition 29 | 0.4926 |
| Composition 30 | 0.2071 |
| Composition 31 | NA |
| Composition 32 | 68.89 |
| Composition 49 | 11.41 |
| Composition 39 - batch 2 | 67.8567 |
| Composition 40 - batch 2 | 21.1633 |
| Composition 41 - batch 2 | 10.3303 |
| Composition 42 - batch 2 | 22.03 |
| Composition 43 - batch 2 | 1.45557 |
| Composition 44 - batch 2 | 12.434 |
| Composition 45 - batch 2 | 0.9678 |
| Composition 46 - batch 2 | 0.8707 |
| Composition 47 - batch 2 | 15.2607 |
| Composition 48 - batch 2 | 2.53767 |
| Composition 49 - batch 2 | 1.13073 |
| Composition 70 - batch 2 | 2.2758 |
| Composition 71 - batch 2 | 0.65927 |
| Composition 72 - batch 2 | 14.9903 |
| Composition 73 - batch 2 | 0.21197 |
| Composition 74 - batch 2 | 1.98633 |
| Composition 75 - batch 2 | 99.05 |
| Composition 76 - batch 2 | 32.0233 |
| Composition 77 - batch 2 | 17.6967 |
| Composition 78 - batch 2 | 19.72 |
| Composition 79 - batch 2 | 3.08067 |
| Composition 80 - batch 2 | 50.83 |
| Composition 81 - batch 2 | 1.41677 |
| Composition 82 - batch 2 | 40.5 |

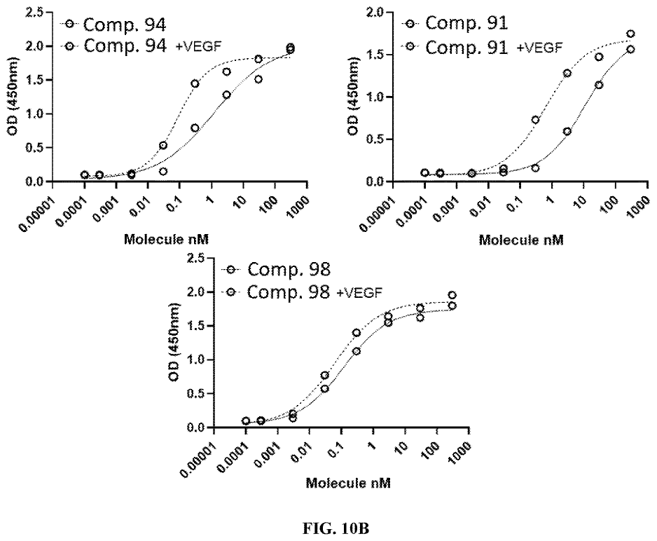

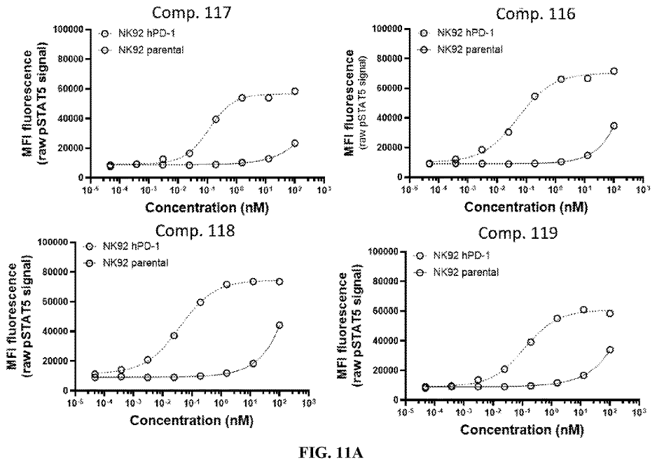

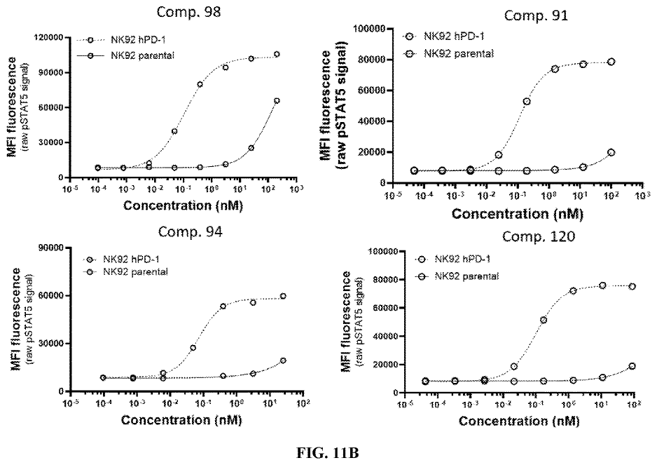

Selected compositions were tested analogously to the above experiment, but both with and without the presence of the recombinant human VEGF 165. These results are shown in FIGS. 10A and 10B and in the Tables below. Graphs show representative dose response curves for selected molecules on HEKBlue® IL2 reporter cells with or without VEGF; Selected molecules show different cooperativity after the addition of VEGF (left shift of curve).

TABLE 34
IL2 Payload Characterization by HEKBlue ®.

| Composition | AVERAGE EC50 | AVERAGE EC50 (nM) with |
|---|---|---|
| Number | (nM) w/o VEGF | VEGF |
| Composition 116 | 2.398 | 0.236 |
| Composition 117 | 0.383 | 0.641 |
| Composition 118 | 2.445 | 1.375 |
| Composition 119 | 0.188 | 0.177 |

TABLE 35
Additional IL2 Payload Characterization by HEKBlue ®.

| Composition | AVERAGE EC50 | AVERAGE EC50 |
|---|---|---|
| Number | (nM) w/o VEGF | (nM) with VEGF |
| Composition 98 | 0.134 | 0.067 |
| Composition 94 | 0.819 | 0.066 |
| Composition 91 | 10.980 | 0.629 |

### Example 3B—STAT5 Activation Assay of NK92 Parental and PD1⁺ Cells

### Protocol for NK92 Parental and Engineered NK92 Overexpressing Human PD1 Assay Analyzing STAT5 Activation as a Readout for IL2 Activity

An experiment was performed to assess the ability of compositions described herein to stimulate STAT5 induction in parental NK92 cells (PD1⁻) and NK92 cells engineered to overexpress human PD-1.

The following materials were used:
Cell Lines: NK92 PD-1—parental cell line (ATCC) and NK92 PD-1⁺ cell line (transduced with human PD-1).
Culture Medium: NK92 cells were maintained in 75% α-MEM (with ribo- and deoxyribonucleosides) supplemented with 12.5% heat-inactivated FBS, 12.5% heat-inactivated horse serum, 2 mM L-glutamine, and 20 ng/mL IL-2. For PD-1⁺ cells, 10 μg/mL Blasticidin was added.
Buffers and Reagents: DPBS++(Gibco, #14040174); BD Pharmingen™ Transcription Factor Phospho Buffer Set (Fix/Perm Kit, BD #563239).
Antibody: Anti-pSTAT5 (BD #562075).
Equipment: NovoCyte Quanteon flow cytometer (Agilent)

Cell Resting (Day: −1)—NK92 cells were collected in 50 mL conical tubes and washed three times with 50 mL DPBS (350×g, 4 min) to remove residual IL-2. Cells were resuspended in NK92 culture medium without IL-2 (and without Blasticidin for PD-1⁺ cells) at a density of 0.3×10⁶ cells/mL and rested overnight at 37° C.

Cell Preparation and Compound Stimulation (Day: 0)—Rested NK92 cells were washed twice with 50 mL DPBS (350×g, 4 min) and resuspended in assay medium (PBS(++)+0.1% BSA) at a density of 0.1×10⁶ cells/20 μL per well. A 2× compound titration was prepared in assay medium, starting at 100 nM with a 1:8 dilution factor. 20 μL of compound solution and 20 μL of cell suspension were added per well. Plates were incubated for 40 min at 37° C.

Fixation and Permeabilization—Fifteen minutes before use, 1×TFP Fix/Perm buffer (4° C.) was prepared by diluting 4× stock in 0.75×TFP diluent, and 1× Wash buffer was prepared from 5×Perm/Wash stock using bidistilled water. To stop stimulation, 200 μL/well of 1× Fix/Perm buffer (4° C.) was added, and plates were incubated for 50 min at 4° C. Cells were centrifuged (500×g, 4 min, 4° C.), washed twice with 150 μL Wash buffer (4° C.), and resuspended in 200 μL PermBuffer III (−20° C.) for 20 min on ice or at −20° C.

Intracellular Staining and Flow Cytometry—After permeabilization, cells were centrifuged (500×g, 4 min, 4° C.), washed twice with Wash buffer, and resuspended in 40 μL antibody mix (anti-pSTAT5 in Wash buffer). Staining was performed for 60 mi at 4° C., followed by two washes with Wash buffer and one wash with FACS buffer (4° C.). Finally, cells were resuspended in 26 μL FACS buffer, and 20 μL was acquired on a NovoCyte Quanteon flow cytometer.

Table 35 summarizes average EC50 of all molecules tested on parental NK92 and NK92 engineered to overexpress human PD1. EC50 are calculated based on pSTAT5 MFI and percentage of pSTAT5 positive cells. At least n=2 if not otherwise stated.

TABLE 35
IL2 Payload Screen In NK92 Cells

| | | | | | | | Average | |
|---|---|---|---|---|---|---|---|---|
| | Average | | Average | | Average | | EC50 (nM) | stdv |
| | EC50 (nM) | stdv | EC50 (nM) | stdv | EC50 (nM) | stdv | NK92 | NK92 |
| | NK92 | NK92 | NK92 | NK92 | NK92 | NK92 | PD1 + | PD1 + |
| Composition | parental | parental | PD1 + | PD1 + | parental | parental | AA | AA |
| Number | MFI | MFI | AA MFI | AA MFI | frequency | frequency | frequency | frequency |
| Composition 1 | 3.10194 | 1.5501 | 0.02471 | 0.01224 | 0.51537 | 0.22105 | 0.00571 | 0.00242 |
| Composition 2 | 3.51139 | 3.35322 | 0.03187 | 0.01608 | 0.3881 | 0.12767 | 0.01114 | 0.00602 |
| Composition 3 | 146.15385 | 50.83911 | 0.17531 | 0.19457 | 104.60909 | 52.26085 | 0.09405 | 0.13462 |
| Composition 4 | 1.72007 | 1.11548 | 0.02017 | 0.00068 | 0.58893 | 0.22721 | 0.00611 | 0.00298 |
| composition 217 | 1.875 | 0.31451 | 0.11342 | 0.04425 | 3.92467 | 0.9525 | 0.15021 | 0.05711 |
| composition 219 | 1.72333 | 0.10796 | 0.03013 | 0.009 | 3.72833 | 0.84171 | 0.0379 | 0.01161 |
| composition 218 | 39.75967 | 52.16996 | 0.02063 | 0.00521 | 18.90667 | 5.69019 | 0.03293 | 0.00403 |
| composition 220 | 8.24233 | 3.37297 | 0.02108 | 0.0073 | 13.88333 | 3.32339 | 0.03144 | 0.00978 |
| composition 221 | 1.36025 | 0.11602 | 0.01516 | 0.00626 | 2.56675 | 0.41595 | 0.01685 | 0.00836 |
| composition 222 | 6.58775 | 1.84851 | 0.01503 | 0.00742 | 11.90025 | 2.39773 | 0.01868 | 0.00938 |
| Composition 52 | 150.04714 | 62.82614 | 0.19334 | 0.09691 | 37.406 | 5.41192 | 0.05967 | 0.02365 |
| Composition 55 | 162.684 | 83.44111 | 0.07449 | 0.0374 | 25.105 | 10.10456 | 0.02521 | 0.00532 |
| Composition 55 | 175 | 50 | 0.12564 | 0.09693 | 200 | only 1 | 0.0921 | only 1 |
| | | | | | | repeat | | repeat |
| Composition 58 | 200 | 0 | 0.13139 | 0.10368 | 200 | only 1 | 0.04044 | only 1 |
| | | | | | | repeat | | repeat |
| Composition 83 | 160 | 54.77226 | 0.70955 | 0.95757 | 150 | 57.73503 | 0.32319 | 0.43557 |
| Composition 60 | 200 | only 1 | 0.09526 | only 1 | 200 | only 1 | 0.0311 | only 1 |
| | | repeat | | repeat | | repeat | | repeat |
| Composition 62 | 150 | 70.71068 | 0.08112 | 0.0089 | 150 | 70.71068 | 0.01804 | 0.00393 |
| Composition 66 | 166.66667 | 57.73503 | 0.2737 | 0.11 | 122.34 | 69.24758 | 0.06874 | 0.02571 |
| Composition 84 | 200 | only 1 | 0.06866 | only 1 | NA | NA | NA | NA |
| | | repeat | | repeat | | | | |
| Composition 85 | 200 | only 1 | 0.2116 | only 1 | 200 | only 1 | 0.04703 | only 1 |
| | | repeat | | repeat | | repeat | | repeat |
| Composition 91 | 116.66667 | 40.82483 | 0.15882 | 0.09157 | 90.40167 | 23.51102 | 0.09373 | 0.06435 |
| Composition 94 | 100.01167 | 141.40485 | 0.072 | only 1 | 8.6428 | 12.20071 | 0.01072 | only 1 |
| | | | | repeat | | | | repeat |
| Composition 98 | 200 | 0 | 0.08282 | 0.03255 | 15.92333 | 3.60895 | 0.01703 | 0.00158 |
| Composition 116 | 100 | 0 | 0.31874 | 0.39572 | 100 | 0 | 0.06825 | 0.05698 |
| Composition 117 | 100 | 0 | 0.82047 | 0.88682 | 100 | 0 | 0.26131 | 0.19587 |
| Composition 118 | 100 | 0 | 0.40904 | 0.656 | 100 | 0 | 0.0622 | 0.02386 |
| Composition 119 | 100 | 0 | 0.54313 | 0.4409 | 100 | 0 | 0.50449 | 0.22639 |
| Composition 120 | 100 | 0 | 0.12116 | 0.04605 | 100 | 0 | 0.12021 | 0.05723 |

FIG. 11A shows dose response curves of selected molecules on parental NK92 and NK92 overexpressing human PD 1; Curves show avidity driven activity gain of the IL2 payloads in the presence of PD1 as shown by the increase in pSTAT5 signal. Without PD1 (parental cells) no STAT5 activation is seen. pSTAT5 signal shown as MFI.

FIG. 11B show dose response curves of selected molecules on parental NK92 and NK92 overexpressing human PD 1; Curves show avidity driven activity gain of the IL2 payloads in the presence of PD1 as shown by the increase in pSTAT5 signal. Without PD1 (parental cells) no STAT5 activation is seen. pSTAT5 signal shown as MFI.

### Example 3C—Characterization of IL2 Payloads Via BLI

### Protocol for BLI, Binding to IL2 Receptor Trimer

The following materials were used:
Bio-Layer Interferometry (BLI) experiments were performed using Octet® R8 system (Sartorius) equipped with High Precision Streptavidin Biosensors (SAX2) (Sartorius, Cat. No. 18-5136). The following biotinylated ligands were used:
IL-2Rα (CD25): Biotinylated Human IL-2 R alpha/CD25 Protein, His, Avitag™, premium grade (AcroBiosystems, #ILA-H82E6), reconstituted at 200 μg/mL.
IL-2Rβ (CD122): Biotinylated Human IL-2 R beta/CD122 Protein, His, Avitag™ (AcroBiosystems, #ILB-H82E3).
IL-2Rγ (CD132): Biotinylated Human IL-2 R gamma/CD132 Protein, His, Avitag™ (AcroBiosystems, #ILG-H85E8).
IL-2Rβγ heterodimer: Biotinylated Human IL-2 R beta & IL-2 R gamma Heterodimer Protein, Fc, Avitag™ (AcroBiosystems, #ILG-H82F3).
IL-2Rαβγ heterotrimer: Biotinylated Human IL-2 R beta & IL-2 R alpha & IL-2 R gamma Protein, Fc, Avitag™ (AcroBiosystems, #ILG-H82F4).

Analytes consisted of the indicated compositions prepared by serial dilution. The assay was performed in 1× Kinetic Buffer (PBS, 0.1% BSA, 0.02% Tween-20). Regeneration buffer was 10 mM Glycine, pH 2.06. All measurements were conducted in 96-well polypropylene flat-bottom plates (Greiner, #655209).

Binding kinetics were assessed using Bio-Layer Interferometry (BLI). Ligand loading solutions were prepared at a concentration of 0.3 pg/mL for subunits or 0.6 pg/mL for heterodimers in a total volume of 2 mL, and 200 μL of this solution was dispensed into each well.

Analyte solutions were prepared by performing a 1:2.5 serial dilution, starting from an initial concentration of 200 nM to achieve a final volume of 200 μL per well.

Kinetic measurements were conducted using the following BLI program: ligand loading for 600 s, followed by three regeneration cycles (6 s/6 s), association for 200 s, dissociation for 200 s, and a final three regeneration cycles (6 s/6 s).

Data analysis was performed using Octet Analysis Software. Binding curves were fitted using a 1:1 global model with an unlinked curve fit to determine the equilibrium dissociation constant (K_D).

Table 36 summarizes the KDs of selected molecules for 112 receptor monomers, dimers and trimer, molecules show different affinities to the different receptor subunits.

TABLE 36
BLI Measurements of IL-2R Subunit Binding

| | Composition | Sensor | KD | KD | ka | kdis |
|---|---|---|---|---|---|---|
| Sensor Type | Number | Info | (M) | (nM) | (1/Ms) | (1/s) |
| SAX2 (High | Composition | alpha | 2.91E−08 | 29.05 | 4.73E+05 | 1.37E−02 |
| Precision | 116 | | | | | |
| Streptavidin 2.0) | | | | | | |
| SAX2 (High | Composition | beta | no | no | | |
| Precision | 116 | | binding | binding | | |
| Streptavidin 2.0) | | | | | | |
| SAX2 (High | Composition | bg | no | no | | |
| Precision | 116 | | binding | binding | | |
| Streptavidin 2.0) | | | | | | |
| SAX2 (High | Composition | abg | 1.51E−09 | 1.51 | 3.38E+06 | 5.12E−03 |
| Precision | 116 | | | | | |
| Streptavidin 2.0) | | | | | | |
| SAX2 (High | Composition | alpha | 1.42E−08 | 14.17 | 4.87E+05 | 6.90E−03 |
| Precision | 117 | | | | | |
| Streptavidin 2.0) | | | | | | |
| SAX2 (High | Composition | beta | no | no | | |
| Precision | 117 | | binding | binding | | |
| Streptavidin 2.0) | | | | | | |
| SAX2 (High | Composition | bg | no | no | | |
| Precision | 117 | | binding | binding | | |
| Streptavidin 2.0) | | | | | | |
| SAX2 (High | Composition | abg | 2.74E−09 | 2.74 | 1.39E+06 | 3.80E−03 |
| Precision | 117 | | | | | |
| Streptavidin 2.0) | | | | | | |
| SAX2 (High | Composition | alpha | 1.78E−08 | 17.82 | 5.48E+05 | 9.76E−03 |
| Precision | 118 | | | | | |
| Streptavidin 2.0) | | | | | | |
| SAX2 (High | Composition | beta | no | no | | |
| Precision | 118 | | binding | binding | | |
| Streptavidin 2.0) | | | | | | |
| SAX2 (High | Composition | bg | no | no | | |
| Precision | 118 | | binding | binding | | |
| Streptavidin 2.0) | | | | | | |
| SAX2 (High | Composition | abg | 1.03E−09 | 1.03 | 4.49E+06 | 4.64E−03 |
| Precision | 118 | | | | | |
| Streptavidin 2.0) | | | | | | |
| SAX2 (High | Composition | alpha | 7.46E−09 | 7.46 | 3.73E+05 | 2.78E−03 |
| Precision | 119 | | | | | |
| Streptavidin 2.0) | | | | | | |
| SAX2 (High | Composition | beta | no | no | | |
| Precision | 119 | | binding | binding | | |
| Streptavidin 2.0) | | | | | | |
| SAX2 (High | Composition | bg | no | no | | |
| Precision | 119 | | binding | binding | | |
| Streptavidin 2.0) | | | | | | |
| SAX2 (High | Composition | abg | 1.21E−09 | 1.21 | 1.47E+06 | 1.78E−03 |
| Precision | 119 | | | | | |
| Streptavidin 2.0) | | | | | | |
| SAX2 (High | Composition | alpha | 3.27E−08 | 32.71 | 1.44E+05 | 4.72E−03 |
| Precision | 120 | | | | | |
| Streptavidin 2.0) | | | | | | |
| SAX2 (High | Composition | beta | no | no | | |
| Precision | 120 | | binding | binding | | |
| Streptavidin 2.0) | | | | | | |
| SAX2 (High | Composition | bg | no | no | | |
| Precision | 120 | | binding | binding | | |
| Streptavidin 2.0) | | | | | | |
| SAX2 (High | Composition | abg | 2.10E−09 | 2.1 | 2.39E+06 | 5.02E−03 |
| Precision | 120 | | | | | |
| Streptavidin 2.0) | | | | | | |
| SAX2 (High | Composition | alpha | 3.10E−08 | 31.01 | 1.22E+05 | 3.79E−03 |
| Precision | 91 | | | | | |
| Streptavidin 2.0) | | | | | | |
| SAX2 (High | Composition | beta | no | no | | |
| Precision | 91 | | binding | binding | | |
| Streptavidin 2.0) | | | | | | |
| SAX2 (High | Composition | bg | no | no | | |
| Precision | 91 | | binding | binding | | |
| Streptavidin 2.0) | | | | | | |
| SAX2 (High | Composition | abg | 3.59E−09 | 3.59 | 1.18E+06 | 4.23E−03 |
| Precision | 91 | | | | | |
| Streptavidin 2.0) | | | | | | |
| SAX2 (High | Composition | alpha | 4.71E−08 | 47.1 | 1.52E+05 | 7.17E−03 |
| Precision | 94 | | | | | |
| Streptavidin 2.0) | | | | | | |
| SAX2 (High | Composition | beta | no | no | | |
| Precision | 94 | | binding | binding | | |
| Streptavidin 2.0) | | | | | | |
| SAX2 (High | Composition | bg | 4.45E−07 | 445.34 | 8.23E+04 | 3.67E−02 |
| Precision | 94 | | | | | |
| Streptavidin 2.0) | | | | | | |
| SAX2 (High | Composition | abg | 1.51E−09 | 1.51 | 2.45E+06 | 3.70E−03 |
| Precision | 94 | | | | | |
| Streptavidin 2.0) | | | | | | |
| SAX2 (High | Composition | alpha | 1.13E−08 | 11.34 | 1.78E+05 | 2.02E−03 |
| Precision | 98 | | | | | |
| Streptavidin 2.0) | | | | | | |
| SAX2 (High | Composition | beta | no | no | | |
| Precision | 98 | | binding | binding | | |
| Streptavidin 2.0) | | | | | | |
| SAX2 (High | Composition | bg | no | no | | |
| Precision | 98 | | binding | binding | | |
| Streptavidin 2.0) | | | | | | |
| SAX2 (High | Composition | abg | 2.29E−09 | 2.29 | 1.03E+06 | 2.36E−03 |
| Precision | 98 | | | | | |
| Streptavidin 2.0) | | | | | | |

### Example 4A—PD-1 Characterization—BLI Analyzing Binding to PD1

Protocol for bivalent PD-1 binding on SAX2 sensors by BLI The following materials were used: Bio-Layer Interferometry experiments were performed on an Octet® R8 system (Sartorius) using High Precision Streptavidin Biosensors (SAX2) (Sartorius, Cat. No. 18-5136). The ligand was biotinylated human PD-1/PDCD1 protein (Avitag™, His Tag, 18.6 kDa; AcroBiosystems, #PD1-H82E4), prepared at a fixed concentration of 0.0375 pg/mL. Analytes consisted of the indicated compositions prepared by serial dilution. The assay was conducted in 1× Kinetic Buffer (PBS, 0.1% BSA, 0.02% Tween-20), with 10 mM glycine (pH 2.06) as the regeneration buffer. All measurements were performed in 96-well polypropylene flat-bottom plates (Greiner, #655209).

Ligand loading solutions were prepared at a concentration of 0.0375 pg/mL in a total volume of 2 mL, and 200 μL was dispensed into each well. Analyte solutions were prepared by 1:2 serial dilution, starting from 30 nM, to a final volume of 200 μL per well.

Kinetic measurements were performed using the following BLI program:Loading: 600 s; Regeneration: 3 cycles of 6 s/6 s; Association: 300 s; Dissociation: 500 s; Regeneration: 3 cycles of 6 s/6 s.

Data were analyzed using Octet Analysis Software, applying a 1:1 global model with an unlinked curve fit to determine the equilibrium dissociation constant (K_D).

Table 37 shows data on PD-1 binding analyzed by BLI (bivalent). All selected molecules show binding to PD1 when analyzed via BLI.

TABLE 37
PD-1 binding data analyzed by BLI

| Sensor | | Composition | KD | KD | ka | kdis |
|---|---|---|---|---|---|---|
| Type | Sensor Info | Number | (M) | (nM) | (1/Ms) | (1/s) |
| SAX2 (High | Biotinylated | Composition | 2.14E−11 | 0.021 | 9.33E+05 | 1.99E−05 |
| Precision | PD1 | 91 | | | | |
| Streptavidin 2.0) | | | | | | |
| SAX2 (High | Biotinylated | Composition | 1.00E−12 | 0.001 | 7.75E+05 | 1.53E−07 |
| Precision | PD1 | 91 | | | | |
| Streptavidin 2.0) | | | | | | |
| SAX2 (High | Biotinylated | Composition | 1.00E−12 | 0.001 | 5.53E+05 | 2.49E−07 |
| Precision | PD1 | 98 | | | | |
| Streptavidin 2.0) | | | | | | |
| SAX2 (High | Biotinylated | Composition | 1.25E−09 | 1.246 | 1.36E+06 | 1.70E−03 |
| Precision | PD1 | 116 | | | | |
| Streptavidin 2.0) | | | | | | |
| SAX2 (High | Biotinylated | Composition | 1.53E−09 | 1.528 | 1.17E+06 | 1.79E−03 |
| Precision | PD1 | 117 | | | | |
| Streptavidin 2.0) | | | | | | |
| SAX2 (High | Biotinylated | Composition | 1.14E−09 | 1.138 | 1.56E+06 | 1.77E−03 |
| Precision | PD1 | 118 | | | | |
| Streptavidin 2.0) | | | | | | |
| SAX2 (High | Biotinylated | Composition | 1.75E−09 | 1.754 | 1.04E+06 | 1.82E−03 |
| Precision | PD1 | 119 | | | | |
| Streptavidin 2.0) | | | | | | |
| SAX2 (High | Biotinylated | Composition | 4.64E−11 | 0.046 | 1.00E+06 | 4.64E−05 |
| Precision | PD1 | 120 | | | | |
| Streptavidin 2.0) | | | | | | |

### Example 4B—PD1 Blocking Assay

### Protocol for PD1 Blocking Assay

The ability of the control and multifunctional immunocytokines to interfere with PD1/PDL1 pathway was measured using the PD-1/PD-L1 Blockade Bioassay from Promega (Cat. #J1250, Madison, WI, USA). PD-1/PD-L1 Blockade Bioassay is a bioluminescent cell-based assay based on the co-culture of effector cells with target cells mimicking an immunological synapse. Jurkat T cells expressing human PD-1 (Jurkat-Lucia-PD1) and a luciferase reporter driven by a NFAT response element (NFAT-RE) are activated by CHO-K1 cells expressing human PD-L1 (Raji-APC-L1, Cat #Rajkt-hpd1, Invivogen) and an engineered cell surface protein designed to activate Jurkat's cognate TCRs. Concurrent interaction PD-1/PD-L1 inhibits TCR signaling and represses NFAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture 96-well plates and cultured 24 h at 37° C./5% CO₂. Test molecules were measured in serial dilutions starting at and pre-incubated on target cells for 10 min before the addition of freshly thawed PD-1 Jurkat effector cells. Samples were incubated either with or without VEGFA (recombinant human VEGF165 (VE5-H5248, Acro Bio)) in a 2:1 molar ratio with immunocytokine. After 24 h at 37° C./5% CO₂, activity NFAT-RE luminescent reporter was evaluated by removing a 20 microliter sample to which QUANTI-Lic reagent (#rep-qlc, Invivogen) was added. Bioluminescence was then immediately read with a plate reader

Table 38 shows average IC50s of selected molecules; IC50s decrease with the addition of VEGF suggesting cooperativity.

TABLE 38
PD1 Blocking Assay Results

| | Composition | Composition | Composition | Composition | Composition | Composition | Composition | Composition |
|---|---|---|---|---|---|---|---|---|
| Composition | 116 | 116 | 117 | 117 | 118 | 118 | 119 | 119 |
| Average | 30.18 | 2.234 | 34.81 | 3.7325 | 36.805 | 2.4645 | 52.835 | 2.766 |
| IC50 nM | | | | | | | | |

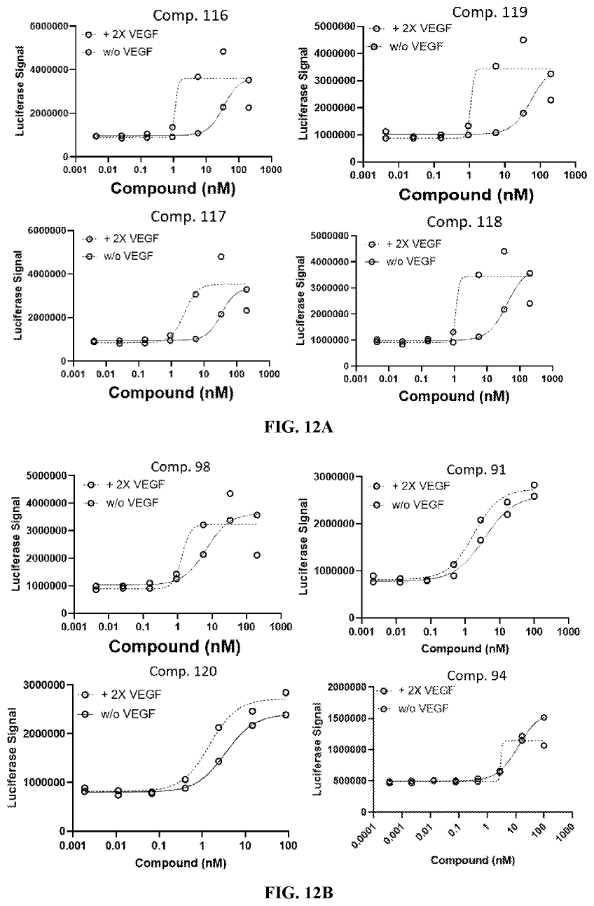

FIG. 12A shows representative dose response curves of PD1 blocking by selected molecules, blocking ability is increased (left shift) by the addition of VEGF suggesting cooperativity. FIG. 12B shows representative dose response curves of PD1 blocking by selected molecules. Table 39 shows average IC50s of selected molecules.

TABLE 39
PD1 Blockade Assay Results

| | | Composition | | Composition | | Composition | | Composition |
|---|---|---|---|---|---|---|---|---|
| | Composition | 98 + 2x | Composition | 91 + 2x | Composition | 94 + 2x | Composition | 120 + 2X |
| Composition | 98 | VEGF | 91 | VEGF | 94 | VEGF | 120 | VEGF |
| Average | 5.466 | 2.457 | 4.06 | 1.6513 | 8.192 | 2.902 | 6.0547 | 1.5589 |
| IC50 nM | | | | | | | | |

### Example 4C—Screening of Additional PD-1 Binding Domains

Additional candidate anti-PD-1 binding domains provided herein were screened for activity. In this example, all candidate binders were screened in the format of FIG. 2C. Each molecule containing the indicated candidate anti-PD-1 VHH comprised a light chain of SEQ ID NO: 285 and a heavy chain comprising, in an N-terminal to C-terminal direction, the sequence of SEQ ID NO: 170; the sequence of SEQ ID NO: 30, and the relevant VHH (as defined in Table 1C).

### Biolayer Interferometry (BLI)

BLI experiments were performed to assess the ability of anti-PD-1 binding domain VHHs using PD-1 as ligand bound to the biosensor (“PD1 as ligand”) and with PD-1 in solution with dual binding composition bound to the biosensor (“PD1 monovalent”).

### PD1 as Ligand Materials and Methods

PD1 as ligand: Materials: Bio-Layer Interferometry experiments were performed on an Octet® R8 system (Sartorius) using High Precision Streptavidin Biosensors (SAX2) (Sartorius, Cat. No. 18-5136). The ligand was biotinylated human PD-1/PDCD1 protein (Avitag™, His Tag, 18.6 kDa; AcroBiosystems, #PD1-H82E4), prepared at a fixed concentration of 0.0375 μg/mL. Analytes consisted of the indicated compositions prepared by serial dilution. The assay was conducted in 1× Kinetic Buffer (PBS, 0.1% BSA, 0.02% Tween-20), with 10 mM glycine (pH 2.06) as the regeneration buffer. All measurements were performed in 96-well polypropylene flat-bottom plates (Greiner, #655209).

PD1 as ligand: Methods. Ligand loading solutions were prepared at a concentration of 0.0375 μg/mL in a total volume of 2 mL, and 200 μL was dispensed into each well. Analyte solutions were prepared by 1:2 serial dilution, starting from 30 nM, to a final volume of 200 μL per well. Kinetic measurements were performed using the following BLI program: Loading: 600 s; Regeneration: 3 cycles of 6 s/6 s; Association: 300 s; Dissociation: 500 s; Regeneration: 3 cycles of 6 s/6 s. Data were analyzed using Octet Analysis Software, applying a 1:1 global model with an unlinked curve fit to determine the equilibrium dissociation constant (K_D).

### PD1 Monovalent Materials and Methods

PD1 monovalent: Materials. Experiments were performed on an Octet® R8 system (Sartorius) using Anti-Human Fc Capture (AHC) Biosensors (Sartorius, Cat. No. 18-5063). Ligands consisted of the indicated compositions immobilized at a fixed concentration of 0.6 μg/mL. The analyte was recombinant human PD-1, His-tagged protein (R&D Systems, #8986-PD), prepared by serial dilution. The assay was conducted in 1× Kinetic Buffer (PBS, 0.1% BSA, 0.02% Tween-20) in 96-well polypropylene flat-bottom plates (Greiner, #655209).

PD1 monovalent: Methods. Ligand loading solutions were prepared at 0.6 μg/mL in a total volume of 2 mL, and 200 μL was dispensed into each well. Analyte solutions were prepared by 1:2 serial dilution, starting from 100 nM, to a final volume of 200 μL per well. Kinetic measurements were performed using the following BLI program: Loading: 180 s; Association: 180 s; Dissociation: 300 s. Data were analyzed using Octet Analysis Software, applying a 1:1 global model with an unlinked curve fit to determine the equilibrium dissociation constant (K_D).

Results from these experiments are shown in Table 40 below.

TABLE 40
KDs of Candidate PD-1 Binders as Measured by BLI

| | | PD1 |
|---|---|---|
| | Ab ligand | ligand |
| | (Monovalent) | KD |
| Binder | KD (nM) | (pM) |
| Pembrolizumab | 5.01 | >1 |
| LZM-009 | 15.22 | 4.8 |
| anti-PD1 | No data | 2432.42 |
| VHH103 | | |
| anti-PD1 | No data | 899.82 |
| VHH104 | | |
| anti-PD1 | No data | 4543.26 |
| VHH105 | | |
| anti-PD1 | No data | 4682.58 |
| VHH106 | | |
| anti-PD1 | No data | no |
| VHH107 | | binding |
| anti-PD1 | No data | 7144.47 |
| VHH108 | | |
| anti-PD1 | No data | 551667 |
| VHH109 | | |
| anti-PD1 | No data | 661.38 |
| VHH110 | | |
| anti-PD1 | No data | 6451.32 |
| VHH111 | | |
| anti-PD1 | No data | 104.56 |
| VHH112 | | |
| anti-PD1 | No data | 1 |
| VHH46 | | |
| anti-PD1 | 8.9 | 5.58 |
| VHH47 | | |
| anti-PD1 | No data | 14.14 |
| VHH48 | | |
| anti-PD1 | 22.84 | 10.07 |
| VHH49 | | |
| anti-PD1 | 8.5 | 761.28 |
| VHH50 | | |
| anti-PD1 | No data | 82.53 |
| VHH51 | | |
| anti-PD1 | No data | 451.04 |
| VHH52 | | |
| anti-PD1 | No data | 1.15 |
| VHH53 | | |
| anti-PD1 | No data | 139.85 |
| VHH54 | | |
| anti-PD1 | 16.75 | 119.76 |
| VHH55 | | |
| anti-PD1 | No data | 146.46 |
| VHH56 | | |
| anti-PD1 | 11.13 | 5.03 |
| VHH57 | | |
| anti-PD1 | No data | no |
| VHH58 | | binding |
| anti-PD1 | 11.35 | 10.56 |
| VHH59 | | |
| anti-PD1 | No data | 8733.45 |
| VHH60 | | |
| anti-PD1 | No data | no |
| VHH61 | | binding |
| anti-PD1 | 16.67 | 1.71 |
| VHH62 | | |
| anti-PD1 | No data | 1.19 |
| VHH63 | | |
| anti-PD1 | No data | 62.81 |
| VHH64 | | |
| anti-PD1 | No data | 1.27 |
| VHH65 | | |
| anti-PD1 | No data | 7209.91 |
| VHH66 | | |
| anti-PD1 | No data | 1 |
| VHH67 | | |
| anti-PD1 | 24.21 | 84.23 |
| VHH68 | | |
| anti-PD1 | No data | 1.04 |
| VHH69 | | |
| anti-PD1 | 17.36 | 27.8 |
| VHH70 | | |
| anti-PD1 | No data | 1.34 |
| VHH71 | | |
| anti-PD1 | 14.48 | 114.02 |
| VHH72 | | |
| anti-PD1 | No data | 50.93 |
| VHH73 | | |
| anti-PD1 | No data | 1179.6 |
| VHH74 | | |
| anti-PD1 | 28.85 | 16.36 |
| VHH75 | | |
| anti-PD1 | 153.5 | 15.08 |
| VHH76 | | |
| anti-PD1 | No data | 1 |
| VHH77 | | |
| anti-PD1 | No data | 708.62 |
| VHH78 | | |
| anti-PD1 | No data | 101.27 |
| VHH79 | | |
| anti-PD1 | No data | no |
| VHH80 | | binding |
| anti-PD1 | 25584.78* | 14.03 |
| VHH81 | | |
| anti-PD1 | 60.37 | 24.46 |
| VHH82 | | |
| anti-PD1 | No data | 1 |
| VHH83 | | |
| anti-PD1 | 27.81 | 106.52 |
| VHH84 | | |
| anti-PD1 | No data | no |
| VHH85 | | binding |
| anti-PD1 | No data | 10730.5 |
| VHH86 | | |
| anti-PD1 | No data | 37.16 |
| VHH87 | | |
| anti-PD1 | No data | 293.61 |
| VHH88 | | |
| anti-PD1 | 45.01 | 59.27 |
| VHH89 | | |
| anti-PD1 | No data | 18714.8 |
| VHH90 | | |
| anti-PD1 | No data | 985.64 |
| VHH91 | | |
| anti-PD1 | No data | 1288.95 |
| VHH92 | | |

### Anti-PD-1 ELISA

The interaction of the candidate binder compositions with PD-1 (CD279) were measured by ELISA assay. For these studies, Corning high-binding half-area plates (Fisher Scientific, Reinach, Switzerland) were coated overnight at 4° C. with 25 μl of dual binder compositions at 2.5 μg/ml in PBS. Plates were then washed four times with 100 μl of PBS-0.2% Tween20 (wash buffer). Plate surfaces were blocked with 25 μl of PBS-1% BSA at 37° C. for 1h. Plates were then washed four times with 100 μl of wash buffer. Twenty-five microliters of recombinant biotinylated PD1/CD279 protein (Biotinylated Recombinant Human PD-1 (CD279)-Fc Chimera (carrier-free), Biolegend #799506) were added in serial dilutions in PBS-T with 0.1% BSA and incubated at 37° C. for 2h. Plates were then washed four times with wash buffer. Twenty-five microliters of Streptavidin-Horseradish peroxidase (#RABHRP3, Merck, Buchs, Switzerland) diluted at 1:500 in PBS-0.02% Tween20-0.1% BSA were added to each well and incubated at Room Temperature for 30 min. Plates were then washed four times with 100 Id of wash buffer. Fifty microliters of TMB substrate reagent (#CL07, Merck, Buchs, Switzerland) were added to each well and incubated at 37° C. during 5 min. After 5 min at 37° C., Horseradish peroxidase reaction was stopped by adding 50 l/well of 0.5M H₂SO₄ stop solution. ELISA signal was then measured at 450 nm on an EnSpire plate reader from Perkin Elmer (Schwerzenbach, Switzerland). Results are shown in Table 41 below.

TABLE 41
KDs of Candidate PD-1 Binders as Measured by ELISA

| | Median |
|---|---|
| | KD |
| Binder | (nM) |
| Pembrolizumab | 0.0966 |
| LZM-009 | |
| anti-PD1 | 0.5791 |
| VHH103 | |
| anti-PD1 | 1.685 |
| VHH104 | |
| anti-PD1 | 1.697 |
| VHH105 | |
| anti-PD1 | 0.528 |
| VHH106 | |
| anti-PD1 | Unstable |
| VHH107 | |
| anti-PD1 | 0.7711 |
| VHH108 | |
| anti-PD1 | 14.89 |
| VHH109 | |
| anti-PD1 | 0.8125 |
| VHH110 | |
| anti-PD1 | 2.997 |
| VHH111 | |
| anti-PD1 | |
| VHH112 | |
| anti-PD1 | 0.1385 |
| VHH113 | |
| anti-PD1 | 2.767 |
| VHH114 | |
| anti-PD1 | 0.4895 |
| VHH115 | |
| anti-PD1 | Unstable |
| VHH116 | |
| anti-PD1 | Unstable |
| VHH117 | |
| anti-PD1 | 4.769 |
| VHH118 | |
| anti-PD1 | Unstable |
| VHH119 | |
| anti-PD1 | 1.691 |
| VHH120 | |
| anti-PD1 | 11.35 |
| VHH121 | |
| anti-PD1 | 7.642 |
| VHH122 | |
| anti-PD1 | Unstable |
| VHH123 | |
| anti-PD1 | Unstable |
| VHH124 | |
| anti-PD1 | 6.963 |
| VHH125 | |
| anti-PD1 | 8.594 |
| VHH126 | |
| anti-PD1 | 0.299 |
| VHH45 | |
| anti-PD1 | 0.28 |
| VHH46 | |
| anti-PD1 | 0.137 |
| VHH47 | |
| anti-PD1 | 0.164 |
| VHH48 | |
| anti-PD1 | 0.182 |
| VHH49 | |
| anti-PD1 | 0.225 |
| VHH50 | |
| anti-PD1 | 0.158 |
| VHH51 | |
| anti-PD1 | 0.162 |
| VHH52 | |
| anti-PD1 | 0.239 |
| VHH53 | |
| anti-PD1 | 0.152 |
| VHH54 | |
| anti-PD1 | 0.188 |
| VHH55 | |
| anti-PD1 | 0.267 |
| VHH56 | |
| anti-PD1 | 0.287 |
| VHH57 | |
| anti-PD1 | 0.5342 |
| VHH58 | |
| anti-PD1 | 0.249 |
| VHH59 | |
| anti-PD1 | 0.219 |
| VHH60 | |
| anti-PD1 | 0.51515 |
| VHH61 | |
| anti-PD1 | 0.221 |
| VHH62 | |
| anti-PD1 | 0.25 |
| VHH63 | |
| anti-PD1 | 0.267 |
| VHH64 | |
| anti-PD1 | 0.278 |
| VHH65 | |
| anti-PD1 | 0.713 |
| VHH66 | |
| anti-PD1 | 0.164 |
| VHH67 | |
| anti-PD1 | 0.15 |
| VHH68 | |
| anti-PD1 | 0.209 |
| VHH69 | |
| anti-PD1 | 0.162 |
| VHH70 | |
| anti-PD1 | 0.264 |
| VHH71 | |
| anti-PD1 | 0.213 |
| VHH72 | |
| anti-PD1 | 0.222 |
| VHH73 | |
| anti-PD1 | 0.402 |
| VHH74 | |
| anti-PD1 | 0.17 |
| VHH75 | |
| anti-PD1 | 0.18 |
| VHH76 | |
| anti-PD1 | 0.168 |
| VHH77 | |
| anti-PD1 | 0.356 |
| VHH78 | |
| anti-PD1 | 0.22 |
| VHH79 | |
| anti-PD1 | 0.271 |
| VHH80 | |
| anti-PD1 | 0.218 |
| VHH81 | |
| anti-PD1 | 0.177 |
| VHH82 | |
| anti-PD1 | 0.211 |
| VHH83 | |
| anti-PD1 | 0.216 |
| VHH84 | |
| anti-PD1 | no |
| VHH85 | binding |
| anti-PD1 | no |
| VHH86 | binding |
| anti-PD1 | 0.183 |
| VHH87 | |
| anti-PD1 | 0.266 |
| VHH88 | |
| anti-PD1 | 0.236 |
| VHH89 | |
| anti-PD1 | 0.323 |
| VHH90 | |
| anti-PD1 | 0.673 |
| VHH91 | |
| anti-PD1 | 0.258 |
| VHH92 | |

### PD-1/PD-L1 Blockade Assay

The ability of the control and candidate PD-1 binder compositions to interfere with PD1/PDL1 pathway was measured using the PD-1/PD-L1 Blockade Bioassay from Promega (Cat. #J1250, Madison, WI, USA). PD-1/PD-L1 Blockade Bioassay is a bioluminescent cell-based assay based on the co-culture of effector cells with target cells mimicking an immunological synapse. Jurkat T cells expressing human PD-1 (Jurkat-Lucia-PD1) and a luciferase reporter driven by a NFAT response element (NFAT-RE) are activated by CHO-K1 cells expressing human PD-L1 (Raji-APC-L1, Cat #Rajkt-hpd1, Invivogen) and an engineered cell surface protein designed to activate Jurkat's cognate TCRs. Concurrent interaction PD-1/PD-L1 inhibits TCR signaling and represses NFAT-RE-mediated luminescence. Addition of either an anti-PD-1 or anti-PD-L1 antibody that blocks the PD-1/PD-L1 interaction releases the inhibitory signal, restoring TCR activation and resulting in a gain of signal of NFAT-RE luminescent reporter.

Briefly, PD-L1 aAPC/CHO-K1 Target cells were plated in white tissue culture 96-well plates and cultured 24 h at 37° C./5% CO₂. Test molecules were measured in serial dilutions starting at and pre-incubated on target cells for 10 min before the addition of freshly thawed PD-1 Jurkat effector cells. Samples were incubated either with or without VEGFA (recombinant human VEGF165 (VE5-H5248, Acro Bio)) in a 2:1 molar ratio with dual binder composition. After 24 h at 37° C./5% CO₂, activity NFAT-RE luminescent reporter was evaluated by removing a 20 microliter sample to which QUANTI-Lic reagent (#rep-qlc, Invivogen) was added. Bioluminescence was then immediately read with a plate reader. Results are shown in Table 42 below.

TABLE 42
IC₅₀ Values of Candidate Binders in PD-1/PD-L1 Blockade Assay

| | | Median |
|---|---|---|
| | Median | IC50 |
| | adjusted | (nM) + |
| | IC50 | VEGF |
| Binder | (nM)* | 2x |
| Pembrolizumab | 0.9442 | 0.8851 |
| LZM-009 | 2.281 | 1.499 |
| anti-PD1 | Unstable | 115.325 |
| VHH103 | | |
| anti-PD1 | Unstable | 20.61 |
| VHH104 | | |
| anti-PD1 | Unstable | 38.05 |
| VHH105 | | |
| anti-PD1 | Unstable | Unstable |
| VHH106 | | |
| anti-PD1 | Unstable | Unstable |
| VHH107 | | |
| anti-PD1 | Unstable | 5.132 |
| VHH108 | | |
| anti-PD1 | Unstable | Unstable |
| VHH109 | | |
| anti-PD1 | Unstable | 4.615 |
| VHH110 | | |
| anti-PD1 | Unstable | Unstable |
| VHH111 | | |
| anti-PD1 | Unstable | 6.128 |
| VHH112 | | |
| anti-PD1 | Unstable | Unstable |
| VHH113 | | |
| anti-PD1 | Unstable | Unstable |
| VHH114 | | |
| anti-PD1 | Unstable | Unstable |
| VHH115 | | |
| anti-PD1 | Unstable | Unstable |
| VHH116 | | |
| anti-PD1 | Unstable | 15.62 |
| VHH117 | | |
| anti-PD1 | Unstable | Unstable |
| VHH118 | | |
| anti-PD1 | Unstable | Unstable |
| VHH119 | | |
| anti-PD1 | Unstable | Unstable |
| VHH120 | | |
| anti-PD1 | Unstable | Unstable |
| VHH121 | | |
| anti-PD1 | Unstable | Unstable |
| VHH122 | | |
| anti-PD1 | Unstable | Unstable |
| VHH123 | | |
| anti-PD1 | Unstable | 10.37 |
| VHH124 | | |
| anti-PD1 | Unstable | Unstable |
| VHH125 | | |
| anti-PD1 | 10.5 | 2.965 |
| VHH45 | | |
| anti-PD1 | 246.6 | 2.992 |
| VHH46 | | |
| anti-PD1 | 5.433 | 2.933 |
| VHH47 | | |
| anti-PD1 | 2.194 | 2.552 |
| VHH48 | | |
| anti-PD1 | 12.78 | 2.809 |
| VHH49 | | |
| anti-PD1 | 600 | 16.39 |
| VHH50 | | |
| anti-PD1 | 131 | 2.809 |
| VHH51 | | |
| anti-PD1 | 15.36 | 3.573 |
| VHH52 | | |
| anti-PD1 | 294.7 | 3.589 |
| VHH53 | | |
| anti-PD1 | 108.6 | 6.115 |
| VHH54 | | |
| anti-PD1 | 32.18 | 2.423 |
| VHH55 | | |
| anti-PD1 | 624.6 | 3.109 |
| VHH56 | | |
| anti-PD1 | 22 | 2.305 |
| VHH57 | | |
| anti-PD1 | 329.93 | 5.343 |
| VHH58 | | |
| anti-PD1 | 36.44 | 3.992 |
| VHH59 | | |
| anti-PD1 | 255.4 | 10.93 |
| VHH60 | | |
| anti-PD1 | Unstable | 12.3 |
| VHH61 | | |
| anti-PD1 | 24.14 | 2.222 |
| VHH62 | | |
| anti-PD1 | 593.7 | 5.07 |
| VHH63 | | |
| anti-PD1 | 30.56 | 3.002 |
| VHH64 | | |
| anti-PD1 | 94.59 | 4.492 |
| VHH65 | | |
| anti-PD1 | 1080 | 17.25 |
| VHH66 | | |
| anti-PD1 | 203.3 | 3.542 |
| VHH67 | | |
| anti-PD1 | 101.1 | 2.478 |
| VHH68 | | |
| anti-PD1 | 1200 | 4.812 |
| VHH69 | | |
| anti-PD1 | 15.57 | 2.642 |
| VHH70 | | |
| anti-PD1 | 1200 | 5.954 |
| VHH71 | | |
| anti-PD1 | 15.28 | 2.641 |
| VHH72 | | |
| anti-PD1 | 53.95 | 2.793 |
| VHH73 | | |
| anti-PD1 | 156.3 | 4.874 |
| VHH74 | | |
| anti-PD1 | 31.37 | 2.716 |
| VHH75 | | |
| anti-PD1 | 13.08 | 2.552 |
| VHH76 | | |
| anti-PD1 | 57.71 | 1.918 |
| VHH77 | | |
| anti-PD1 | 1200 | 12.72 |
| VHH78 | | |
| anti-PD1 | 286.2 | 8.336 |
| VHH79 | | |
| anti-PD1 | 489.4 | 14.93 |
| VHH80 | | |
| anti-PD1 | 25.03 | 3.363 |
| VHH81 | | |
| anti-PD1 | 35.11 | 2.826 |
| VHH82 | | |
| anti-PD1 | 88.44 | 2.919 |
| VHH83 | | |
| anti-PD1 | 32.85 | 2.898 |
| VHH84 | | |
| anti-PD1 | 1200 | 600 |
| VHH85 | | |
| anti-PD1 | 1200 | 600 |
| VHH86 | | |
| anti-PD1 | 74.23 | 3.157 |
| VHH87 | | |
| anti-PD1 | 1200 | 3.225 |
| VHH88 | | |
| anti-PD1 | 62.72 | 2.777 |
| VHH89 | | |
| anti-PD1 | 1200 | 17.41 |
| VHH90 | | |
| anti-PD1 | 1200 | 15.8 |
| VHH91 | | |
| anti-PD1 | 600 | 13.96 |
| VHH92 | | |

### Example 5A—VEGF Characterization: VEGF Binding by BLI

### Protocol Bivalent VEGF165 Binding on SAX2 Sensors by BLI

The following materials were used. BLI experiments were performed on an Octet® R8 system (Sartorius) using High Precision Streptavidin Biosensors (SAX2) (Sartorius, Cat. No. 18-5136). The ligand was biotinylated human VEGF165 (His, Avitag™, 22.4 kDa; AcroBiosystems, #VE5-H82Q0), prepared at a fixed concentration of 0.0375 μg/mL. Analytes consisted of the indicated compositions prepared by serial dilution. The assay was conducted in 1× Kinetic Buffer (PBS, 0.1% BSA, 0.02% Tween-20), with 10 mM glycine (pH 2.06) as the regeneration buffer. All measurements were performed in 96-well polypropylene flat-bottom plates (Greiner, #655209).

Ligand loading solutions were prepared at 0.0375 μg/mL in a total volume of 2 mL, and 200 μL was dispensed into each well. Analyte solutions were prepared by 1:2 serial dilution, starting from 30 nM, to a final volume of 200 μL per well.

Kinetic measurements were performed using the following BLI program: Loading: 600 s; Regeneration: 3 cycles of 6 s/6 s; Association: 300 s; Dissociation: 500 s; Regeneration: 3; cycles of 6 s/6 s.

Data were analyzed using Octet Analysis Software, applying a 1:1 global model with an unlinked curve fit to determine the equilibrium dissociation constant (K_D).

### Data on Bivalent Binding to VEGF165 by BLI

Table 43 shows data on bivalent binding to VEGF165 by BLI, where all selected molecules show picomolar binding to VEGF165.

TABLE 43
BLI results of VEGF Binding Experiments

| Sensor | | Composition | KD | KD | ka | kdis |
|---|---|---|---|---|---|---|
| Type | Sensor Info | Number | (M) | (nM) | (1/Ms) | (1/s) |
| SAX2 (High | Biotinylated | Composition | 1.00E−12 | 0.001 | 5.17E+05 | 2.34E−07 |
| Precision | VEGF | 91 | | | | |
| Streptavidin 2.0) | | | | | | |
| SAX2 (High | Biotinylated | Composition | 1.00E−12 | 0.001 | 4.20E+05 | 2.22E−07 |
| Precision | VEGF | 94 | | | | |
| Streptavidin 2.0) | | | | | | |
| SAX2 (High | Biotinylated | Composition | 1.00E−12 | 0.001 | 3.69E+05 | 2.34E−07 |
| Precision | VEGF | 98 | | | | |
| Streptavidin 2.0) | | | | | | |
| SAX2 (High | Biotinylated | Composition | 1.36E−10 | 0.136 | 5.95E+05 | 8.12E−05 |
| Precision | VEGF | 116 | | | | |
| Streptavidin 2.0) | | | | | | |
| SAX2 (High | Biotinylated | Composition | 1.61E−10 | 0.161 | 5.95E+05 | 9.55E−05 |
| Precision | VEGF | 117 | | | | |
| Streptavidin 2.0) | | | | | | |
| SAX2 (High | Biotinylated | Composition | 1.81E−11 | 0.018 | 7.62E+05 | 1.38E−05 |
| Precision | VEGF | 118 | | | | |
| Streptavidin 2.0) | | | | | | |
| SAX2 (High | Biotinylated | Composition | 1.00E−12 | 0.001 | 6.29E+05 | 1.00E−07 |
| Precision | VEGF | 119 | | | | |
| Streptavidin 2.0) | | | | | | |
| SAX2 (High | Biotinylated | Composition | 1.00E−12 | 0.001 | 4.58E+05 | 1.61E−07 |
| Precision | VEGF | 120 | | | | |
| Streptavidin 2.0) | | | | | | |

### Example 5B—Bivalent Binding to VEGF165 on AHC Sensors

### Protocol for Bivalent Binding to VEGF165 on AHC Sensors

The following materials were used. BLI experiments were performed on an Octet® R8 system (Sartorius) using Anti-Human Fc Capture (AHC) Biosensors (Sartorius, Cat. No. 18-5063). Ligands consisted of indicated compositions immobilized at a fixed concentration of 0.6 μg/mL. The analyte was human VEGF 165 protein, His-tagged (AcroBiosystems, #VE5-H5248), prepared by serial dilution. The assay was conducted in 1× Kinetic Buffer (PBS, 0.100 BSA, 0.02% Tween-20) in 96-well polypropylene flat-bottom plates (Greiner, #655209).

Ligand loading solutions were prepared at 0.6 μg/mL in a total volume of 2 mL, and 200 μL was dispensed into each well. Analyte solutions were prepared by 1:2 serial dilution, starting from 100 nM, to a final volume of 200 μL per well.

Kinetic measurements were performed using the following BLI program: Loading: 180 s; Association: 180 s; Dissociation: 300 s.

Data were analyzed using Octet Analysis Software, applying a 1:1 global model with an unlinked curve fit to determine the equilibrium dissociation constant (K_D).

Table 44 shows KDs of tested VEGF Nb using BLI (human VEGF165).

TABLE 44
Results of VEGF binding Measured by BLI

| Composition | KD | KD | | kdis |
|---|---|---|---|---|
| Number | (M) | (nM) | ka (1/Ms) | (1/s) |
| Composition | 8.55E− | 0.855 | 1.62E+06 | 1.39E−03 |
| 121 | 10 | | | |
| Composition | 1.11E− | 1.106 | 8.19E+05 | 9.06E−04 |
| 122 | 09 | | | |
| Composition | 1.31E− | 1.305 | 9.00E+05 | 1.17E−03 |
| 123 | 09 | | | |
| Composition | 8.85E− | 0.885 | 1.92E+06 | 1.70E−03 |
| 124 | 10 | | | |
| Composition | 8.65E− | 0.865 | 1.12E+06 | 9.72E−04 |
| 125 | 10 | | | |
| Composition | 1.01E− | 1.01 | 1.78E+06 | 1.80E−03 |
| 126 | 09 | | | |
| Composition | 5.10E− | 5.097 | 4.41E+05 | 2.25E−03 |
| 127 | 09 | | | |
| Composition | 3.39E− | 3.387 | 2.64E+05 | 8.94E−04 |
| 128 | 09 | | | |
| Composition | 1.09E− | 1.094 | 1.33E+06 | 1.45E−03 |
| 129 | 09 | | | |
| Composition | 6.82E− | 0.682 | 1.69E+06 | 1.15E−03 |
| 130 | 10 | | | |
| Composition | 7.16E− | 0.716 | 1.97E+06 | 1.41E−03 |
| 131 | 10 | | | |
| Composition | 1.21E− | 12.056 | 9.06E+05 | 1.09E−02 |
| 132 | 08 | | | |
| Composition | 1.02E− | 1.02 | 1.81E+06 | 1.85E−03 |
| 133 | 09 | | | |
| Composition | 9.96E− | 0.996 | 1.37E+06 | 1.36E−03 |
| 134 | 10 | | | |
| Composition | 1.61E− | 1.613 | 1.19E+06 | 1.91E−03 |
| 135 | 09 | | | |
| Composition | 1.28E− | 1.28 | 1.00E+06 | 1.28E−03 |
| 136 | 09 | | | |
| Composition | 1.20E− | 1.199 | 1.44E+06 | 1.72E−03 |
| 137 | 09 | | | |
| Composition | 1.02E− | 1.024 | 1.34E+06 | 1.37E−03 |
| 138 | 09 | | | |
| Composition | 8.06E− | 0.806 | 2.06E+06 | 1.66E−03 |
| 139 | 10 | | | |
| Composition | 1.34E− | 1.336 | 1.24E+06 | 1.65E−03 |
| 140 | 09 | | | |
| Composition | 2.46E− | 2.462 | 8.63E+05 | 2.13E−03 |
| 141 | 09 | | | |
| Composition | 3.13E− | 3.126 | 8.06E+05 | 2.52E−03 |
| 142 | 09 | | | |
| Composition | 2.43E− | 2.433 | 6.59E+05 | 1.60E−03 |
| 143 | 09 | | | |
| Composition | 1.41E− | 1.413 | 2.14E+06 | 3.02E−03 |
| 144 | 09 | | | |
| Composition | 2.09E− | 2.089 | 1.20E+06 | 2.50E−03 |
| 145 | 09 | | | |
| Composition | 3.62E− | 3.62 | 1.08E+06 | 3.91E−03 |
| 146 | 09 | | | |
| Composition | 1.22E− | No | | |
| 147 | 04 | binding | | |
| Composition | 3.20E− | No | | |
| 148 | 10 | binding | | |
| Composition | 1.43E− | 1.431 | 1.45E+06 | 2.07E−03 |
| 149 | 09 | | | |
| Composition | 8.96E− | 0.896 | 1.25E+06 | 1.12E−03 |
| 150 | 10 | | | |
| Composition | 1.91E− | 1.906 | 1.03E+06 | 1.96E−03 |
| 151 | 09 | | | |
| Composition | 2.39E− | 2.388 | 5.44E+05 | 1.30E−03 |
| 152 | 09 | | | |
| Composition | 1.40E− | 1.398 | 1.25E+06 | 1.75E−03 |
| 153 | 09 | | | |
| Composition | 1.48E− | 1.481 | 8.67E+05 | 1.28E−03 |
| 154 | 09 | | | |
| Composition | 9.76E− | 0.976 | 2.48E+06 | 2.42E−03 |
| 155 | 10 | | | |
| Composition | 1.15E− | 1.145 | 1.54E+06 | 1.76E−03 |
| 156 | 09 | | | |
| Composition | 6.62E− | 6.616 | 8.09E+05 | 5.35E−03 |
| 157 | 09 | | | |
| Composition | 1.99E− | 1.994 | 9.37E+05 | 1.87E−03 |
| 158 | 09 | | | |
| Composition | 1.44E− | 1.439 | 9.33E+05 | 1.34E−03 |
| 159 | 09 | | | |
| Composition | 2.43E− | 2.434 | 2.35E+06 | 5.72E−03 |
| 160 | 09 | | | |
| Composition | 1.06E− | 1.058 | 1.79E+06 | 1.89E−03 |
| 161 | 09 | | | |
| Composition | 1.60E− | 1.596 | 1.40E+06 | 2.24E−03 |
| 162 | 09 | | | |
| Composition | 1.06E− | 1.058 | 1.44E+06 | 1.52E−03 |
| 163 | 09 | | | |
| Composition | 2.17E− | 2.169 | 8.82E+05 | 1.91E−03 |
| 164 | 09 | | | |
| Composition | 1.24E− | 0.124 | 1.22E+07 | 1.50E−03 |
| 165 | 10 | | | |
| Composition | 8.98E− | 0.898 | 2.19E+06 | 1.97E−03 |
| 166 | 10 | | | |
| Composition | 1.77E− | 1.774 | 1.05E+06 | 1.87E−03 |
| 167 | 09 | | | |
| Composition | 1.14E− | 1.135 | 2.27E+06 | 2.57E−03 |
| 168 | 09 | | | |

Table 45 and show molar KDs for binding to human VEGF 165 of all selected molecules as measured by BLI on AHC sensors.

TABLE 45
Data for binding to human VEGF165 by BLI on AHC
sensors with molar KDs of selected molecules

| Sensor | Composition | KD | KD | ka | kdis |
|---|---|---|---|---|---|
| Type | Number | (M) | (nM) | (1/Ms) | (1/s) |
| AHC | | 1.80E− | 0.002 | 2.79E+ | 5.00E− |
| (Anti- | | 12 | | 05 | 07 |
| hIgG Fc | | | | | |
| Capture) | | | | | |
| AHC | Composition | 1.70E− | 0.17 | 3.00E+ | 5.08E− |
| (Anti- | 94 | 10 | | 05 | 05 |
| hIgG Fc | | | | | |
| Capture) | | | | | |
| AHC | Composition | 1.14E− | 0.001 | 2.99E+ | 3.41E− |
| (Anti- | 98 | 12 | | 05 | 07 |
| hIgG Fc | | | | | |
| Capture) | | | | | |
| AHC | Composition | 1.61E− | 1.609 | 6.29E+ | 1.01E− |
| (Anti- | 116 | 09 | | 05 | 03 |
| hIgG Fc | | | | | |
| Capture) | | | | | |
| AHC | Composition | 1.15E− | 1.154 | 8.20E+ | 9.46E− |
| (Anti- | 117 | 09 | | 05 | 04 |
| hIgG Fc | | | | | |
| Capture | | | | | |
| AHC | Composition | 5.30E− | 0.53 | 7.73E+ | 4.10E− |
| (Anti- | 118 | 10 | | 05 | 04 |
| hIgG Fc | | | | | |
| Capture) | | | | | |
| AHC | Composition | 6.74E− | 0.673 | 8.63E+ | 5.81E− |
| (Anti- | 119 | 10 | | 05 | 04 |
| hIgG Fc | | | | | |
| Capture) | | | | | |
| AHC | Composition | 7.41E− | 0.074 | 3.26E+ | 2.41E− |
| (Anti- | 120 | 11 | | 05 | 05 |
| hIgG Fc | | | | | |
| Capture | | | | | |

### Example 5C—VEGF Characterization: VEGF Blocking Assay

### Protocol for VEGFR Blocking Assay

The following materials were used:
VEGF Bioassay Kit (Promega, Cat. #GA2001), containing:
KDR/NFAT-RE HEK293 cells
Assay Medium
VEGF Standard
Control Antibody
Bio-Glo™ Luciferase Assay Reagent
96-well white tissue culture plates (recommended for luminescence)
CO₂ incubator (37° C., 5% CO₂)
Luminometer (e.g., GloMax® Discover, Promega

The VEGF Bioassay was performed according to the manufacturer's instructions (Promega Technical Manual TM544). Briefly, cryopreserved KDR/NFAT-RE HEK293 cells were thawed and resuspended in assay medium. Cells were plated into 96-well white plates at the recommended density and incubated overnight at 37° C. in a humidified atmosphere with 5% CO₂.

On the following day, serial dilutions of VEGF standard or test samples were prepared in assay medium. For neutralization studies, VEGF was pre-incubated with anti-VEGF antibodies for 30 minutes at room temperature before addition to the cells. Cells were treated with 100 μL of VEGF dilutions or sample mixtures and incubated for 6 hours at 37° C., 5% CO₂.

After incubation, 100 μL of Bio-Go™ Luciferase Assay Reagent was added directly to each well. Plates were incubated for 5-10 minutes at room temperature to stabilize the luminescent signal. Luminescence was measured using a GloMax® Discover luminometer. Data were analyzed by plotting relative luminescence units (RLU) versus VEGF concentration to generate a dose-response curve.

Table 46 shows average IC50s of selected molecules; all molecules show sub-nanomolar IC50s for the inhibition of VEGFR signaling

TABLE 46
Average IC50s of selected molecules for VEGFR blocking assay.

| | | | number |
|---|---|---|---|
| Composition | average | | of |
| Number | IC50 (nM) | stdv | repeats |
| Composition | 0.2294 | 0.08 | 8 |
| 116 | | | |
| Composition | 0.2422 | 0.144 | 4 |
| 117 | | | |
| Composition | 0.1688 | 0.089 | 5 |
| 118 | | | |
| Composition | 0.2086 | 0.046 | 5 |
| 119 | | | |
| Composition | 0.5549 | 0.014 | 2 |
| 94 | | | |
| Composition | 0.5517 | 0.131 | 2 |
| 120 | | | |
| Composition | 0.5491 | 0.082 | 2 |
| 91 | | | |

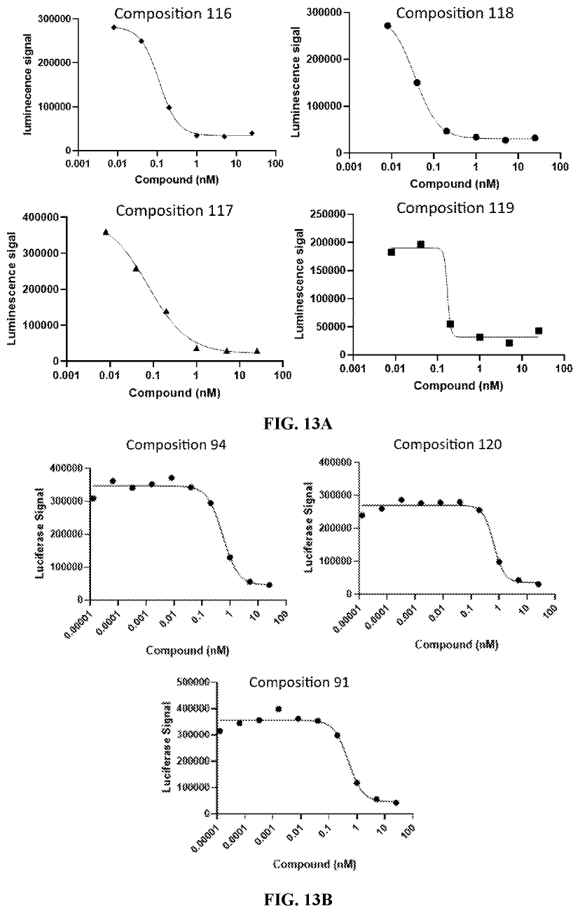

FIG. 13A-13B show representative dose titration curves of selected molecules. All molecules showed sub-nanomolar IC50s for the inhibition of VEGFR signaling.

### Example 5D—Additional Anti-VEGFA Activity Analysis

### ELISA

The specific implementation process of ELISA is as follows: Recombinant human VEGF (Beijing Sino Biological Inc., Catalog No. 11066-HNAH) or mouse VEGF (Beijing Sino Biological Inc., Catalog No. 50159-MNAB) is coated on a 96-well high-adsorption ELISA plate with a carbonate buffer solution of pH=9.6, the coating concentration was 1 μg/mL, 100 μL per well. The coating s carried out at 4° C. overnight, washed 5 times with PBST, blocked with PBST containing 5% skim milk and 1% BSA at 300 L/well, and incubated at 25° C. for one hour, washed 5 times with PBST. Samples of heterodimeric antibodies serially diluted in PBST containing 1% BSA and controls are added, 100 μL was added to each well, and incubated at 25° C. for 1 hour, washed 5 times with PB ST. Then, horseradish peroxidase-labeled anti-human IgG antibody (Chemicon, Catalog No. AP309P) diluted 1:10000 in PBST containing 1% BSA is added, 100 μL was added to each well, and incubated at 25° C. for 1 hour. Washed 5 times with PBST. Colorimetric substrate TMB is added, 100 L/well, color is developed at room temperature for 10 minutes. 1M H2SO4 s added, 100 L/well, the color development is terminated. The absorbance at 450 nm is read on the microplate reader.

### VEGF Neutralization Assay

The proliferation of human umbilical vein endothelial cells (HUVEC) is regulated by VEGF. The primary cell was used to measure the VEGF neutralizing activity of the anti-PD-1/VEGF heterodimeric antibodies.

Human umbilical vein endothelial cells (HUVEC) are obtained from PromoCell (Catalog No: C-12203). HUVEC cells are cultured in a cell incubator with ECGM-2 medium (PromoCell, Catalog No. C-22011), 37° C., 5% CO2. The analysis medium is ECBM-2 (PromoCell, Catalog No: C-22211) medium containing 0.4% FBS (Hyclone, Catalog No: SH30084.03). The HUVEC are collected by trypsin digestion, washed twice with ECBM-2 medium, resuspended in the analysis medium, the cell density is 1×10E5/mL, and is inoculated at 100 L/well on a 96-well cell culture plate, i.e. 10,000 cells per well. 50 μL/well of samples of heterodimeric antibodies serially diluted with analysis medium and controls are added, then 50 L/well of human VEGF (Beijing Sino Biological Inc., Catalog No. 11066-HNAH) diluted with analysis medium is added, and the final concentration is 50 ng/mL. The culture plate is placed in a 37° C., 5% CO2 incubator and incubated for 3 days. At the end of the incubation, 40 μL of MTS (CellTiter96 Aqueous One Solution, Promega, Catalog No: G358B) is added to each well of the cell culture plate to detect cell viability. The cell culture plate is incubated in the incubator for 3-4 hours, and then the absorbance at 490 nm on the microplate reader is read.

### Example 6 Primary Cell Assays

Protocol for Human PBMC Activation and pSTAT5 Assay to Analyze IL2 Payload Activity

The following materials were used/Peripheral blood mononuclear cells (PBMCs) were obtained from the Blutspendezentrum Aarau (Aarau, Switzerland). T cell culture medium consisted of RPMI 1640 (Gibco, Cat. No. 21875034) supplemented with 10% fetal bovine serum (FBS), 1% non-essential amino acids (NEAA; Gibco, Cat. No. 11140), and 25 μM β-mercaptoethanol (Gibco, Cat. No. 31350-010). Staphylococcal enterotoxin B (SEB) was purchased from Sigma-Aldrich (Cat. No. h4881-5MG). Phospho-specific fixation and permeabilization were performed using the BD Pharmingen™ Transcription Factor Phospho Buffer Set (BD Biosciences, Cat. No. 563239). Dulbecco's phosphate-buffered saline with calcium and magnesium (DPBS++; Gibco, Cat. No. 14040174) was used for washing steps. Table 47 shows the antibody panel.

TABLE 47
Antibody Panel

| Target | Clone | Fluorochrome | Vendor | Catalog No. |
|---|---|---|---|---|
| pSTAT5 | 47 | Alexa Fluor ® | BD | 562077 |
| (pY694) | | 647 | Biosciences | |
| CD3 | UCHT1 | FITC | BioLegend | 300306 |
| CD4 | RPA-T4 | PE | BioLegend | 300548 |
| CD8 | RPA-T8 | PerCP-Cy5.5 | BioLegend | 344714 |
| CD14 | M5E2 | BV421 | BioLegend | 301815 |
| CD19 | HIB19 | BV510 | BioLegend | 302238 |
| CD56 | HCD56 | PE-Cy7 | BioLegend | 318334 |
| CD25 | M-A251 | APC | BD | 552852 |
| | | | Biosciences | |
| Pre-staining | | | | |
| panel: | | | | |
| CD4 (pre) | RPA-T4 | PE | BioLegend | 300548 |
| CD8 (pre) | RPA-T8 | PerCP-Cy5.5 | BioLegend | 344714 |
| CD14 (pre) | M5E2 | BV421 | BioLegend | 301815 |
| CD16 | 3G8 | BV605 | BioLegend | 302038 |
| CD20 | 2H7 | BV711 | BioLegend | 302342 |
| CD45RA | HI100 | BV785 | BioLegend | 304138 |
| CD45RO | UCHL1 | PE-Cy7 | BioLegend | 304230 |
| CD62L | DREG-56 | APC | BioLegend | 304814 |
| CD127 | A019D5 | BV650 | BioLegend | |

Protocol for pSTAT5 Measurement on PBMCs after SEB Stimulation to Analyze IL2 Payload Activity

PBMC Thawing and SEB Stimulation: Cryopreserved PBMCs (2-3 vials per experiment) were thawed in a 37° C. water bath until partially thawed. One milliliter of pre-warmed T cell medium was added to each cryovial, and cells were transferred to a 50 mL conical tube. The volume was adjusted to 50 mL with T cell medium, followed by centrifugation at 350×g for 4 min. The supernatant was aspirated, and the cell pellet was resuspended in 10 mL T cell medium. Cells were counted and adjusted to 40 mL in T cell medium containing SEB at a final concentration of 0.5 μg/mL (stock: 5 mg/mL). Cells were incubated at 37° C. for 72 h

Resting Phase (Day −1): SEB-stimulated PBMCs were collected, washed twice with T cell medium (350×g, 4 min), and resuspended in 40 mL T cell medium for overnight resting at 37° C. For unstimulated controls, an additional vial of PBMCs from the same donor was thawed and processed as described above, then rested overnight in T cell medium.

Compound Treatment and Fixation (Day 1): Rested PBMCs were washed twice with T cell medium and resuspended in assay buffer (DPBS++ with 0.1% BSA) at a density of 0.1-0.3×10\hat{ }6 cells per 20 μL per well. A 2× compound dilution series was prepared in assay buffer, starting at 500 nM with an 8-fold serial dilution. Twenty microliters of compound solution and L of cell suspension were added per well and incubated for 40 min at 37° C. Reactions were stopped by adding 200 μL of 1× Fix/Perm buffer (prepared from 4× stock diluted in TFP diluent) and incubating for 50 min at 4° C. Cells were centrifuged (500×g, 4 min, 4° C.) and washed twice with 150 μL of 1× Perm/Wash buffer.

Permeabilization and Intracellular Staining: Cells were permeabilized with 200 μL of Perm Buffer III (−20° C.) for 20 min on ice. After centrifugation and washing, cells were stained with a phospho-STAT5 antibody panel (see Table) for 60 min at 4° C. Following two washes with Perm/Wash buffer and one wash with FACS buffer, cells were resuspended in 26 μL FACS buffer and analyzed on a Novocyte Quanteon flow cytometer.

Table 48 shows median EC50s (nM) of pSTAT5 activation (MFI) in CD4 Treg and CD8 memory subpopulations of selected molecules; Selected molecules containing different 112 variants show different EC50s for pSTAT5 activation in CD4Treg and CD8 memory subpopulations.

TABLE 48
pSTAT5 measurement after SEB stimulation of PBMCs

| | CD4 Treg median | CD8 memory | number of |
|---|---|---|---|
| Compositon No. | EC50 (nM) | median EC50 (nM) | donors |
| Composition 91 | 1.85 | 1.95 | 10 |
| Composition 98 | 0.15 | 0.11 | 8 |
| Composition 115 | 0.67 | 0.54 | 12 |
| Composition 116 | 2.97 | 0.48 | 6 |
| Composition 117 | 1.68 | 1.37 | 6 |
| Composition 118 | 0.36 | 0.28 | 6 |
| Composition 119 | 1.01 | 0.55 | 6 |
| Composition 94 | 0.06 | 0.28 | 6 |

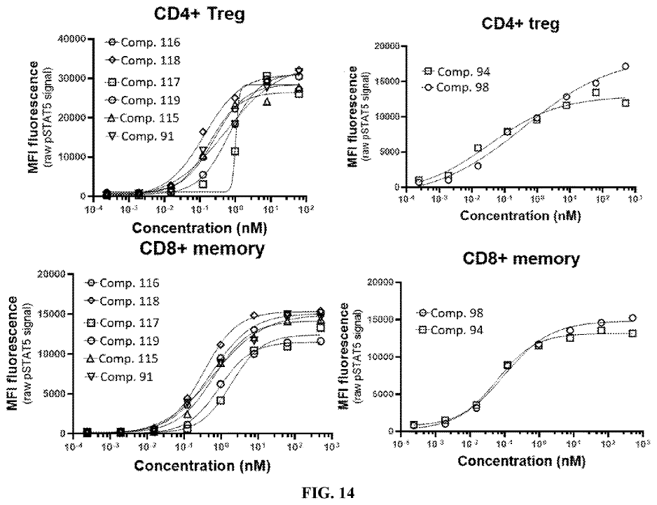

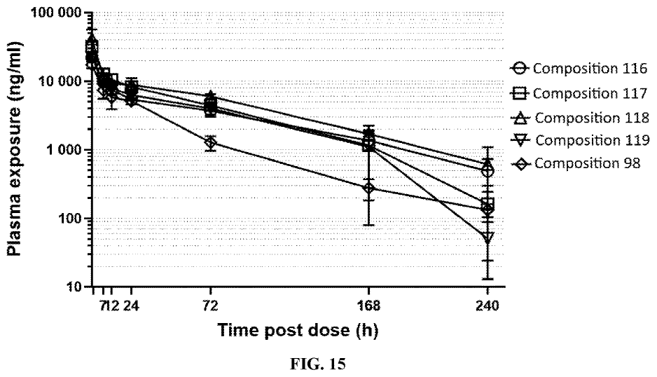

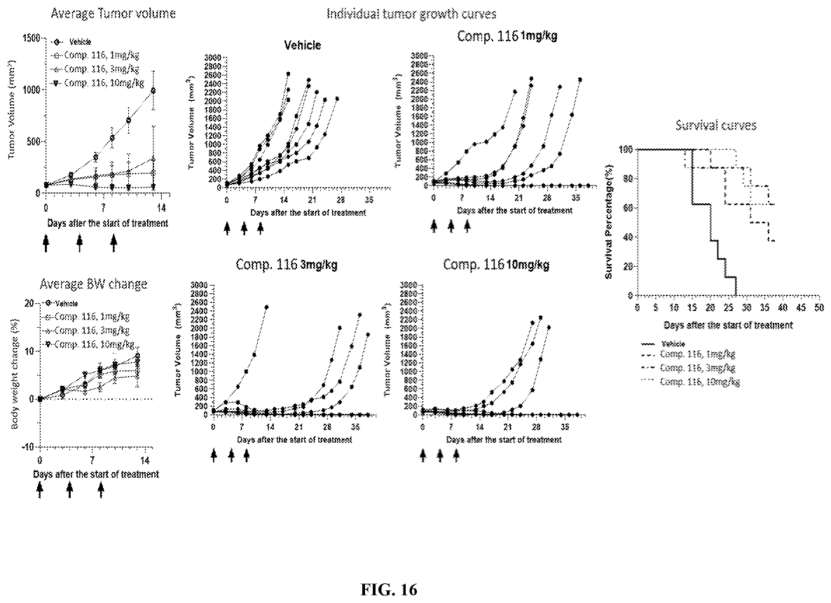

FIG. 14 shows representative dose response curves of pSTAT5 activation (MFI) in CD4 Treg and CD8 memory subpopulations of selected molecules

### Example 7—In Vivo PK

An experiment was performed to assess PK of various immunocytokines. Mice used were huFcRn KI C571BL/6 mice, female, 6-8 weeks, weighing approximately 18-20 g (depending on actual body weight), purchased from Biocytogen Jiangsu Co., Ltd. Table 49 shows Groups and Treatments for in vivo data collection.

TABLE 49
Groups and Treatments

| | | | | Dosing | | |
|---|---|---|---|---|---|---|
| | | | Dose | Volume | Dosing | |
| Group | n | Treatment | (mg/kg) | (μL/g) | Route | Schedule |
| 1 | 6 | Composition | 5 | 10 | i.v. | Single |
| | | 116 | | | bolus | Dose |
| 2 | 6 | Composition | 5 | 10 | i.v. | Single |
| | | 117 | | | bolus | Dose |
| 3 | 6 | Composition | 5 | 10 | i.v. | Single |
| | | 118 | | | bolus | Dose |
| 4 | 6 | Composition | 5 | 10 | i.v. | Single |
| | | 119 | | | bolus | Dose |
| 5 | 6 | Composition | 5 | 10 | i.v. | Single |
| | | 98 | | | bolus | Dose |

Table 50 shows a plasma sampling scheme for all groups.

TABLE 50
Plasma Sampling Scheme for all groups

| | Hours post-dose | | | | | | | | | |
|---|---|---|---|---|---|---|---|---|---|---|
| #mouse | 0.083 | 7 | 12 | 24 | 72 | 168 | 240 | 336 | 408 | 504 |
| 1 | X | | X | | X | | X | | X, terminal | |
| 2 | X | | X | | X | | X | | X, terminal | |
| 3 | X | | X | | X | | X | | X, terminal | |
| 4 | | X | | X | | X | | X | | X, terminal |
| 5 | | X | | X | | X | | X | | X, terminal |
| 6 | | X | | X | | X | | X | | X, terminal |

### Protocol for BA Assay (ELISA) to Detect Trispecific Molecules in Mouse Plasma

The following ELISA Reagents were used.
Coating Reagents: Human VEGF165 Protein, His tag, Acro Bio VE5-H5248
Detection Reagent: rH PD-1(CD279) biotinylated, fc tag R&D BT1086 #62
Buffers: Coating buffer: PBS−/−; Wash buffer: PBS-0.02% Tween20; Blocking buffer: PBS-0.02% Tween20 1% BSA; Protein diluent: PBS-0.02% Tween20 0.10% BSA; STOP solution: 1M H₂SO₄

High-binding ELISA plates were coated with 25 μL per well of human VEGF165 protein (Acro Biosystems, His-tag) diluted in PBS and followed by an overnight incubation at 4° C. Plates were washed four times with PBS containing 0.02% Tween-20 and subsequently blocked with 50 μL per well of PBS supplemented with 1% BSA for 1 h at 37° C. After blocking, plates were washed again four times with wash buffer. Quality controls, calibration standards, and samples were prepared in 10% matrix. 25 μL of each sample were added to the designated wells and incubated overnight at room temperature. Plates were then washed four times, and 25 μL of biotinylated recombinant human PD-1 (R&D Systems) was added to each well, followed by a 1 h incubation at room temperature. After washing, 25 μL of Streptavidin-HRP (1:300 dilution in protein diluent) was added and incubated for 30 min at room temperature. Plates were washed four times before adding 25 μL of TMB substrate (Sigma) to each well and incubating for 5 min at room temperature. The reaction was stopped by adding 25 μL of 1 M H₂SO₄, and absorbance was measured at 450 nm using an EnSpire plate reader.

### Data for PK Analysis

FIG. 15 shows data for PK analysis. All selected molecules showed expected PK in huFcRn KI C57BL/6 mice.

### Non-Compartmental Analysis (NCA):

Selected molecules presented PK characteristics in line with multispecific antibody-based molecules. For the NCA, Software was PKanalix2024R1, license purchased from SimulationPlus. The data showed huFcRn KI C57BL/6 mice plasma concentrations of Compositions 116, 117, 118, 119, and 98 at 5 min, 7 h, 12h, 24 h, 72h, 168h, 240 h post dosing. The data reflect composite PK time profiles with n=3 mice per time point. The data was analyzed as follows: Mean PK parameters computed based on the mean composite profile for each treatment group. BLQ data were treated as BLQ/2. The area under the curve (AUC) calculated was based on the linear up log down method. Table 51 shows the results of the NCA.

TABLE 51
Assessment of PK Parameters

| | | Dose | Dosing | CL_obs | AUC_{INF —}Obs | Vss_Obs | T½ |
|---|---|---|---|---|---|---|---|
| Group | Treatment | (mg/Kg) | Route | (mL · h⁻¹ · Kg⁻¹) | (h · ug · mL⁻¹) | (mL · Kg⁻¹) | (d) |
| 1 | Composition | 5 | i.v. | 1.30 | 3848 | 107.26 | 2.6 |
| | 116 | | bolus | | | | |
| 2 | Composition | 5 | i.v. | 1.14 | 4405 | 63.98 | 1.5 |
| | 117 | | bolus | | | | |
| 3 | Composition | 5 | i.v. | 1.85 | 2701 | 89.73 | 1.6 |
| | 118 | | bolus | | | | |
| 4 | Composition | 5 | i.v. | 1.40 | 3561 | 77.18 | 1.2 |
| | 119 | | bolus | | | | |
| 5 | Composition | 5 | i.v. | 1.88 | 2666 | 101.81 | 1.6 |
| | 98 | | bolus | | | | |

### Example 8 In Vivo-TGI in MC38 Tumors; Mice Transgenic for hPD1

### Protocol for TGI Study in MC38 Bearing C57Bl/6 Mice Transgenic for Human PD1

Cell Culture: The MC38 tumor cells (NTCC) were maintained in vitro as a monolayer culture in DMEM medium supplemented with 10% fetal bovine serum, 100 U/mL penicillin and 100 μg/mL streptomycin at 37° C. in an atmosphere of 5% CO₂ in air. The tumor cells were routinely subcultured twice weekly. The cells growing in an exponential growth phase were harvested and counted for tumor inoculation.

Animals: Mice used were hPD1 KI C57BL/6, female, 6-8 weeks, weighing approximately 18-22 g

Tumor Inoculation: Each mouse was inoculated subcutaneously at the right upper flank with MC38 tumor cells (3×10⁵) in 0.1 mL of PBS for tumor development. The animals were randomized and treatment was started when the average tumor volume reaches approximately 80-120 mm³.

Table 52 shows the groups and treatments for the TGI study in MC38 tumors bearing CB57BI/6 mice transgenic for human PD1.

TABLE 52
Groups and Treatments for the TGI study in MC38 tumors
bearing CB57BI/6 mice transgenic for human PD1

| | | | Dose | | Dosing | Total |
|---|---|---|---|---|---|---|
| Group | -n- | Treatment | Level | ROA | schedule | doses |
| 1 | 8 | Vehicle | — | i.v. | Day 0, Day 4 | 2 |
| | | | | | and Day 8 | |
| 2 | 8 | Composition | 1 | i.v. | Day 0, Day 4 | 2 |
| | | 116 | mg/kg | | and Day 8 | |
| 3 | 8 | Composition | 3 | i.v. | Day 0, Day 4 | 2 |
| | | 116 | mg/kg | | and Day 8 | |
| 4 | 8 | Composition | 10 | i.v. | Day 0, Day 4 | 2 |
| | | 116 | mg/kg | | and Day 8 | |
| 5 | 8 | Composition | 1 | i.v. | Day 0, Day 4 | 2 |
| | | 117 | mg/kg | | and Day 8 | |
| 6 | 8 | Composition | 3 | i.v. | Day 0, Day 4 | 2 |
| | | 117 | mg/kg | | and Day 8 | |
| 7 | 8 | Composition | 10 | i.v. | Day 0, Day 4 | 2 |
| | | 117 | mg/kg | | and Day 8 | |

Endpoints: Tumor sizes were measured three times per week in two dimensions using a caliper, and the volume will be expressed in mm³ using the formula: V=0.5 a×b² where a and b are the long and short diameters of the tumor, respectively.

### Data for TGI Study in MC38 Bearing C57BI 6 Mice Transgenic for Human PD1

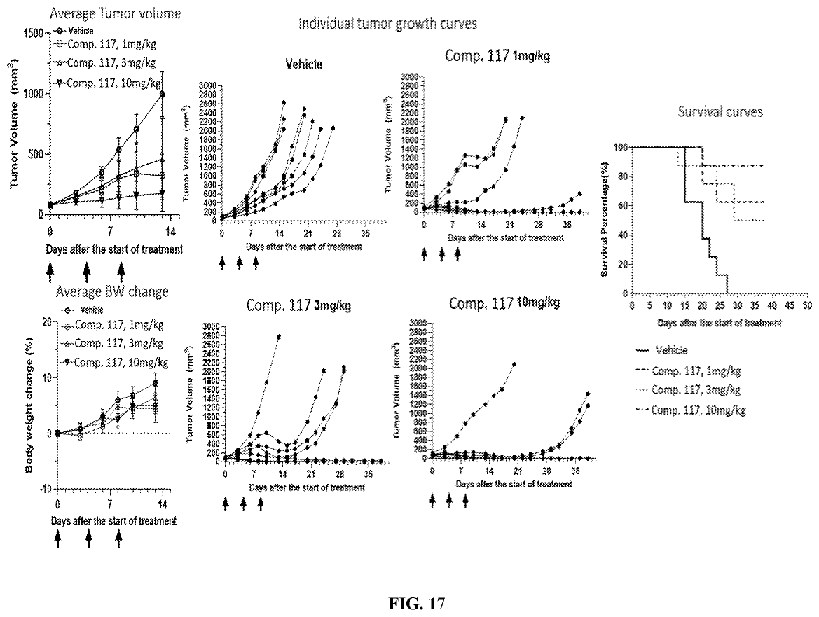

As shown in FIG. 16 and FIG. 17, PD1×VEGF×IL2 trispecific molecule shows a dose dependent anti-tumor efficacy and enables a survival benefit; no BW loss was observed; black arrows indicate treatment days. FIGS. 16 and 17 show the average tumor volume, individual tumor growth curves, and average BW change for the TGI study in MC38 bearing C57Bl/6 mice transgenic for human PD1. FIG. 16 shows data for Composition 116 and FIG. 17 shows data for Composition 117.

### Example 9—In Vivo-TGI in MKN45 CDX in Balb C Nude Mice

### Protocol for MKN45 CDX Study in BalbC Nude Mice

Cell Culture: The MKN45 tumor cells (JCRB) were maintained in vitro as a monolayer culture in RPMI-1640 medium supplemented with 10% fetal bovine serum, 100 U/mL penicillin and 100 μg/mL streptomycin at 37° C. in an atmosphere of 5% CO₂ in air. The tumor cells were routinely subcultured twice weekly. The cells growing in an exponential growth phase were harvested and counted for tumor inoculation

Animals: Mice used were BALB/c nude, female, 6-8 weeks, weighing approximately 18-22 g.

Tumor Inoculation: Each mouse was inoculated subcutaneously at the right upper flank with MKN45 tumor cells (5×10⁶) in 0.1 mL of PBS for tumor development. The animals were randomized and treatment was started when the average tumor volume reached approximately 100-150 mm³

Table 53 shows the groups and treatments for the MKN45 CDX stud in BalbC/nude mice.

TABLE 53
Dosing Schedule

| | | | Dose | | Dosing | Number of |
|---|---|---|---|---|---|---|
| Group | -n- | Treatment | Level | ROA | schedule | injections |
| 1 | 8 | Vehicle | — | i.v. | D 0, 4, 7, | 6 |
| | | | | | 11, 14, 18 | |
| 2 | 8 | Composition | 10 | i.v. | D 0, 4, 7, | 6 |
| | | 117 | mg/kg | | 11, 14, 18 | |
| 3 | 8 | Composition | 10 | i.v. | D 0, 4, 7, | 6 |
| | | 118 | mg/kg | | 11, 14, 18 | |
| 4 | 8 | Composition | 10 | i.v. | D 0, 4, 7, | 6 |
| | | 119 | mg/kg | | 11, 14, 18 | |
| 5 | 8 | Composition | 10 | i.v. | D 0, 4, 7, | 6 |
| | | 300 | mg/kg | | 11, 14, 18 | |
| 6 | 8 | Composition | 10 | i.v. | D 0, 7, 14, | 6 |
| | | 119 | mg/kg | | 21, 28, 35 | |

Endpoints: The major endpoint was to see if the tumor growth can be delayed or mice can be cured. Tumor sizes will be measured twice per week in two dimensions using a caliper, and the volume will be expressed in mm3 using the formula: V=0.5 a×b2 where a and b are the long and short diameters of the tumor, respectively.

Composition 300 is bispecific anti-PD-1/VEGFA construct having the format of FIG. 8A in which the IL-2 polypeptide is replace with an additional anti-PD-1 Fab. The Fabs are that of Pembrolizumab and the anti-VEGF VHHs are VHH175 described herein. Composition 300 has a light chain sequence of SEQ ID NO: 47; a first heavy chain sequence of SEQ ID NO: 163, and a second heavy chain sequence of SEQ ID NO: 162.

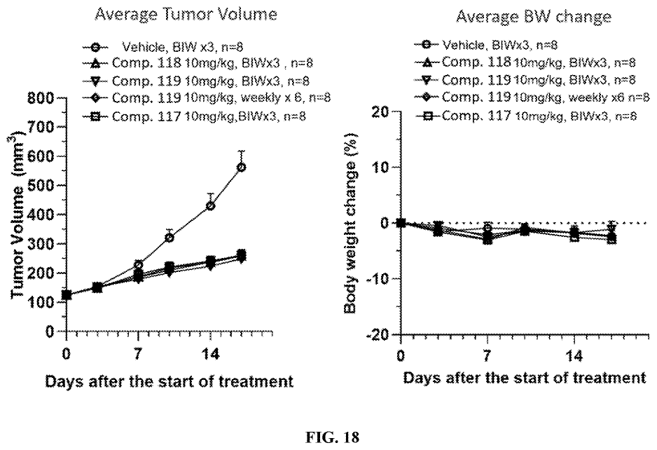

FIG. 18 shows plots of the average tumor volume and average body weight (BW) change vs. the number of days after the start of treatment.

### Example 10—Fc Binding Activity of Immunocytokines

### Human FcγR Binding Assay

The interaction of the unmodified and of conjugated Fc domains (e.g., antibody formats described herein prior to conjugation with cytokines as described herein) with human Fc gamma receptors I (FcγRI/CD64), with human Fc gamma receptors IIa (FcγRIIa/CD32a), with inhibitory human Fc gamma receptors IIb (FcγRIIb/CD32b), and with human Fc gamma receptors III FcγRIIIa/CD16 is measured by ELISA.

Briefly, Corning high-binding half-area plates (Fisher Scientific, Reinach, Switzerland) are coated overnight at 4° C. with 25 μl of unmodified and of conjugated anti-PD1 antibodies at 2.5 g/ml in PBS. Plates are then washed four times with 100 μl of PBS-0.02% Tween20. Plates surfaces are blocked with 25 μl of PBS-0.02% Tween20-1% BSA at 37° C. during 1h. Plates are then washed four times with 100 μl of PBS-0.02% Tween20. Then twenty-five microliters of either recombinant Human Fc gamma RI/CD64 Protein (R&D systems, 1257-FC-050, CF), recombinant Human Fc gamma RIIA/CD32a (H167) Protein (R&D systems, 9595-CD-050, CF), recombinant Human Fc gamma RIIB/CD32b Avi-tag Protein (R&D systems, AVI1875-050, CF), or recombinant Human Fc gamma RIIIA/CD16a Protein (R&D systems, 4325-FC-050; CF) are added in five-fold serial dilutions ranging from 1000 nM to 0.001 nM into PBS-0.02% Tween20-0.1% BSA and incubated at 37° C. during 2h. Plates are then washed four times with 100 μl of PBS-0.02% Tween20. Twenty-five microliters of a 1/500 HRP-anti-His antibody in PBS—0.02% Tween20—0.1% BSA (R&D systems, anti-HIS—HRP Ab, #MAB050H) ae added to each well and plates are incubated at Room Temperature during 1h. Plates are then washed four times with 100 l of PBS-0.02% Tween20. Fifty microliters of TMB substrate reagent (#CL07, Merck, Buchs, Switzerland) are added to each well and incubated at 37° C. during 5 min. After 5 min at 37° C., Horseradish peroxidase reaction is stopped by adding 50 l/well of 0.5M H2SO4 stop solution. ELISA signal is then measured at 450 nm on an EnSpire plate reader from Perkin Elmer (Schwerzenbach, Switzerland).

### Human FcRn Binding Assay

The interaction of the unmodified and of conjugated anti-PD1 antibodies with the human neonatal Fc receptor (FcRn) at pH 6 is measured using the AlphaLISA® Human FcRn Binding Kit (AL3095C) from Perkin Elmer (Schwerzenbach, Switzerland). The AlphaLISA® detection of FcRn and IgG binding uses IgG coated AlphaLISA® acceptor beads to interact with biotinylated human FcRn captured on Streptavidin-coated donor beads. When reference IgG binds to FcRn, donor and acceptor beads come into proximity enabling the transfer of singlet oxygen that trigger a cascade of energy transfer reactions in the acceptor beads, resulting in a sharp peak of light emission at 615 nm. Addition of a free IgG antibodies into the AlphaLISA® mixture creates a competition for the binding of FcRn to the reference antibody resulting in a loss of signal.

Briefly, test molecules arere measured in serial dilutions starting at 5 uM down to 64 pM and incubated with AlphaLISA® reaction mixture consisting of 800 nM of recombinant biotinylated human FcRn, 40 μg/ml of human IgG conjugated Acceptor beads, and 40 μg/ml of Streptavidin coated Donor beads in pH 6 MES buffer. After 90 min at 23° C. in the dark, AlphaLISA® signal is measured on an EnSpire plate reader (Excitation at 680 nm, Emission at 615 nm) from Perkin Elmer (Schwerzenbach, Switzerland).

Although the present disclosure and its advantages have been described in detail, it should be understood that various changes, substitutions and alterations can be made herein without departing from the spirit and scope of the disclosure as defined in the appended claims.

### NUMBERED EMBODIMENTS

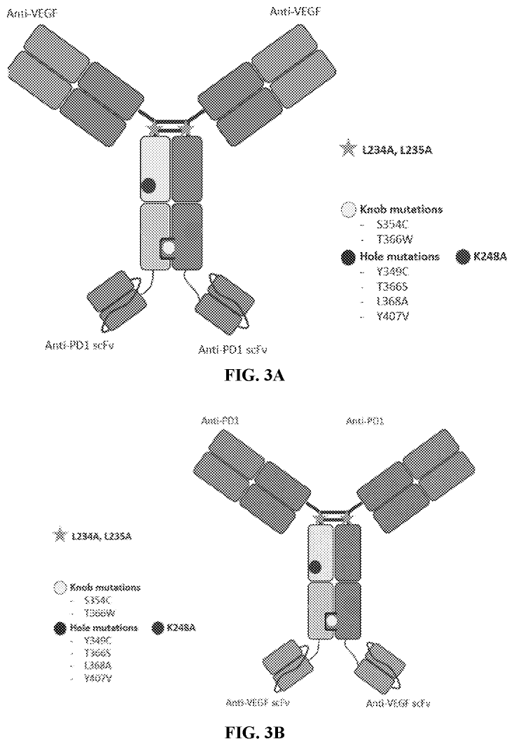

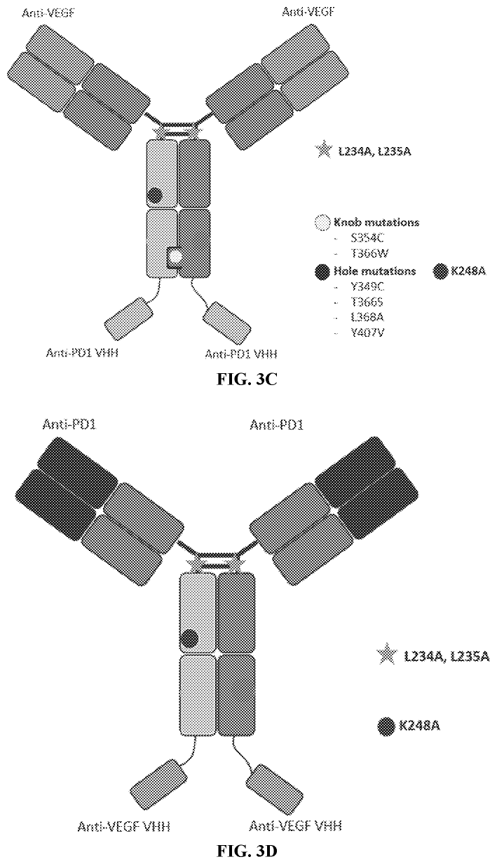

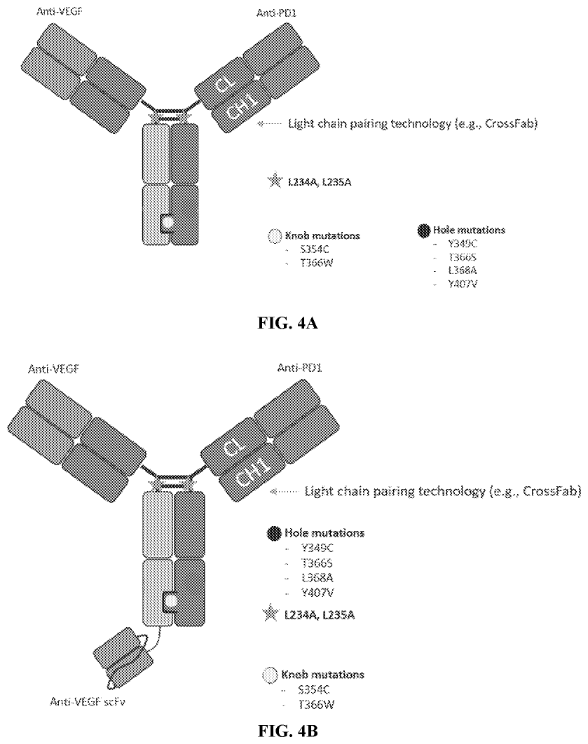

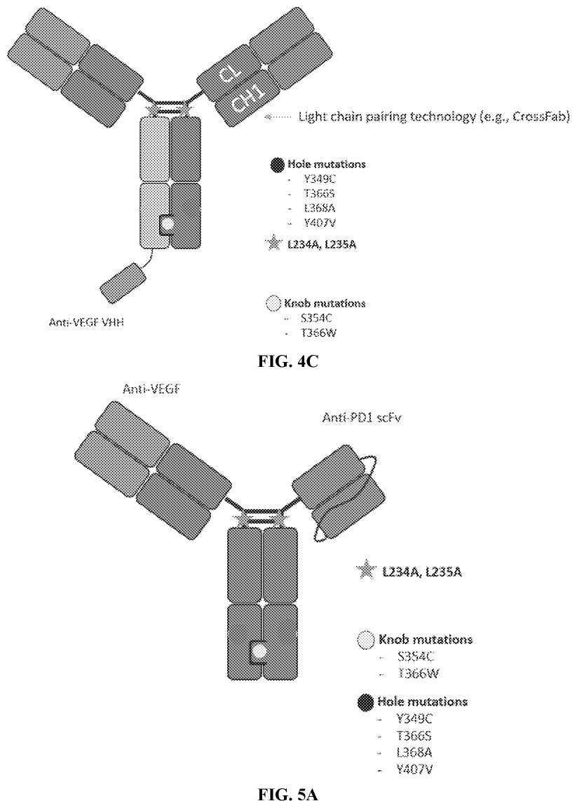

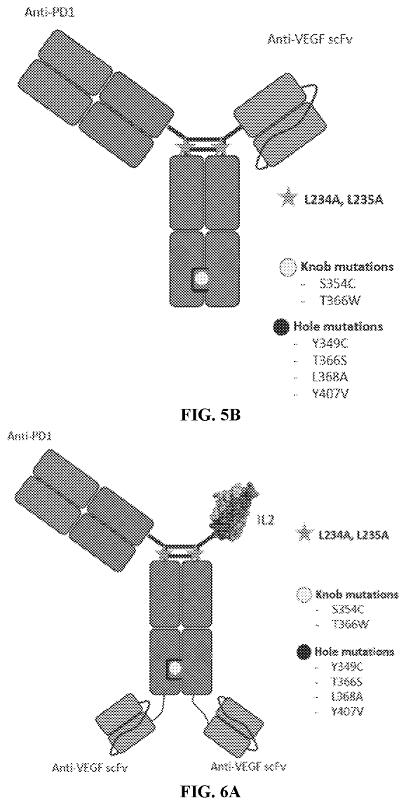

Some numbered examples of embodiments follow. (1). A multifunctional immunocytokine composition, comprising: a) a first binding domain targeting programmed cell death protein 1 (PD-1); b) a second binding domain targeting vascular endothelial growth factor A (VEGFA); and c) a cytokine, wherein each of the first binding domain, the second binding domain, and the cytokine are in covalent association. (2). The composition of embodiment 1, wherein the cytokine is selected from an interleukin, a TNF family cytokine, an interferon, a TGF-b family cytokine, and a chemokine. (3). The composition of embodiment 1 or 2, wherein the cytokine is an IL-2 polypeptide. (4). The composition of embodiment 3, wherein the IL-2 polypeptide comprises an amino acid sequence having at least 80%, 85%, 90%, 95%, 96%, 97%, 98%, 99% or 100% a sequence identity to wild type IL-2 (SEQ ID NO: 701). (5). The composition of embodiment 3 or 4, wherein the IL-2 polypeptide enhanced selectivity for the IL-2 receptor beta subunit over the IL-2 receptor alpha subunit compared to the IL-2 of SEQ ID NO: 701 (wild type IL-2). (6). The composition of any one of embodiments 3-5, wherein the IL-2 polypeptide has reduced affinity for the IL-2 receptor beta subunit compared to the IL-2 of SEQ ID NO: 701. (7). The composition of any one of embodiments 3-6, wherein the IL-2 polypeptide exhibits a reduced ability to signal through the IL-2 receptor beta/gamma complex compared to the IL-2 of SEQ ID NO: 701. (8). The composition of any one of embodiments 3-7, wherein the IL-2 polypeptide comprises at least one polymer covalently attached to a residue selected from residues 35, 37, 38, 41, 42, 43, 44, 45, 60, 61, 62, 64, 65, 68, 69, 71, 72, 104, 105, and 107, wherein residue position numbering is based on SEQ ID NO: 701 as a reference sequence, optionally wherein the polymer forms part of a linker which forms the covalent association with the other portions of the composition. (9). The composition of embodiment 8, wherein, the polymer comprises polyethylene glycol. (10). The composition of embodiment 8 or 9, wherein the IL-2 polypeptide comprises polymers attached at residues 42 and 45. (11). The composition of any one of embodiments 8-10, wherein the IL-2 polypeptide comprises a polymer at residue 42 which forms part of the linker which forms the covalent association with the other portions of the composition. (12). The composition of any one of embodiments 3-11, wherein the IL-2 polypeptide comprises an amino acid sequence having at least about 80%, at least about 85%, at least about 90%, or at least about 95% sequence identity SEQ ID NO: 703, or wherein the IL-2 polypeptide is one described in the subsection titled “Beta-gamma competent IL-2 polypeptides with detuned IL-2 receptor alpha binding”. (13). The composition of embodiment 3 or 4, wherein the IL-2 polypeptide retains the ability to bind to the IL-2 receptor alpha subunit and exhibits a substantially diminished ability to bind to the IL-2 receptor beta or gamma subunits relative to SEQ ID NO: 701 (e.g., such as an IL-2 polypeptide of SEQ ID NO: 775); or wherein the IL-2 polypeptide comprises an amino acid sequence set forth in SEQ ID NO: 775; or wherein the IL-2 polypeptide is one described in the subsection titled “IL-2 Polypeptides Which Selectively Bind to IL-2 Receptor Alpha Subunit”. (14). The composition of embodiment 13, wherein the IL-2 polypeptide comprises one or more of the following modifications: one or more substitutions selected from T3A, Y31H, K35R, Q74P, N88D, N88R, S130R; and/or a substitution of the B′C′ loop of the IL-2 polypeptide with the IL-15B′C′ loop. (15). The composition of embodiment 13, wherein the IL-2 polypeptide comprises the sequence SEQ ID NO: 706. (16). The composition of any one of embodiments 3-15, wherein the IL-2 polypeptide comprises an amino acid sequence set forth in Table 3. (17). The composition of any one of embodiments 3-16, wherein the IL-2 polypeptide is in covalent association with the other portions of the composition via a linker attached to a side chain of an amino acid residue of the IL-2 polypeptide. (18). The composition of embodiment 17, wherein the linker is attached to residue 35, 37, 38, 41, 42, 43, 44, 45, 60, 61, 62, 64, 65, 68, 69, 71, 72, 104, 105, and 107 of the IL-2 polypeptide, wherein residue position numbering is based on SEQ ID NO: 1 as a reference sequence. (19). The composition of embodiment 18, wherein the linker is attached to residue 42 of the IL-2 polypeptide or the N-terminus of the IL-2 polypeptide. (20). The composition of any one of embodiments 17-19, wherein the linker is attached to an Fc region of the composition. (21). The composition of embodiment 20, wherein the linker is attached to the Fc region at a position of a K246 amino acid residue, a K248 amino acid residue, a K288 amino acid residue, a K290 amino acid residue, or a K317 amino acid residue of the Fc region (EU numbering). (22). The composition of embodiment 21, wherein the linker is attached to the K248 residue. (23). The composition of any one of embodiments 3-17, wherein the IL-2 polypeptide is in covalent association via a fusion of the IL-2 polypeptide to the portion of the composition to which it is attached. (24). The composition of any one of embodiments 1-23, wherein the first binding domain targeting programmed cell death protein 1 (PD-1) is capable of disrupting the interaction of PD-1 with programmed cell death ligand 1 (PD-L1). (25). The composition of any one of embodiments 1-24, wherein the first binding domain is an antigen binding fragment derived from an antibody. (26). The composition of any one of embodiments 1-25, wherein the first binding domain comprises a heavy chain variable domain (VH) comprising a heavy chain first complementary determining region (VH CDR1), a heavy chain second complementary determining region (VH CDR2), and a heavy chain third complementary determining region (VH CDR3). (27). The composition of embodiment 26, wherein the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. (28). The composition of embodiment 26 or 27, wherein the VH comprises a set of VH CDR1, VH CDR2, and VH CDR3 derived from an antibody in Table 1A. (29). The composition of any one of embodiments 26-28, wherein the VH comprise an amino acid sequence of a VH set forth in Table 1A. (30). The composition of any one of embodiments 26-29, wherein the first binding domain is a VHH. (31). The composition of any one of embodiments 1-29, wherein the first binding domain comprises a light chain variable domain (VL) comprising a light chain first complementary determining region (VL CDR1), a light chain second complementary determining region (VL CDR2), and a light chain third complementary determining region (VL CDR3). (32). The composition of embodiment 31, wherein the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the first binding domain is comprised. (33). The composition of embodiment 31 or 32, wherein the VL comprises a set of VL CDR1, VL CDR2, and VL CDR3 derived from an antibody in Table 2A; or the composition of any one of embodiments 1-29 or 31-33, wherein the first binding domain is an scFv. (34). The composition of any one of embodiments 1-34, wherein the second binding domain targeting VEGFA is capable of disrupting the interaction of VEGFA with one or more of its receptors. (35). The composition of any one of embodiments 1-35, wherein the second binding domain is comprised in an antigen binding fragment derived from an antibody. (37). The composition of any one of embodiments 1-36, wherein the second binding domain comprises a heavy chain variable domain (VH) comprising a heavy chain first complementary determining region (VH CDR1), a heavy chain second complementary determining region (VH CDR2), and a heavy chain third complementary determining region (VH CDR3). (38). The composition of embodiment 37, wherein the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. (39). The composition of embodiment 37 or 38, wherein the VH comprises a set of VH CDR1, VH CDR2, and VH CDR3 derived from an antibody in Table 2A. (40). The composition of any one of embodiments 37-39, wherein the VH comprise an amino acid sequence of a VH set forth in Table 2A. (41). The composition of any one of embodiments 37-40, wherein the second binding domain is a VHH. (42). The composition of any one of embodiments 1-40, wherein the second binding domain comprises a light chain variable domain (VL) comprising a light chain first complementary determining region (VL CDR1), a light chain second complementary determining region (VL CDR2), and a light chain third complementary determining region (VL CDR3). (43). The composition of embodiment 42, wherein the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the second binding domain is comprised. (44). The composition of embodiment 42 or 43, wherein the VL comprises a set of VL CDR1, VL CDR2, and VL CDR3 derived from an antibody in Table 2A. (45). The composition of any one of embodiments 1-40 or 42-44, wherein the first binding domain is an scFv. (46). The composition of any one of embodiments 1-45, wherein the composition comprises an Fc domain comprising first CH2 and CH3 domains on a first polypeptide chain and second CH2 and CH3 domains on a second polypeptide chain. (47). The composition of embodiment 46, wherein the Fc domain is derived from an IgG. 48). The composition of embodiment 47, wherein the Fc domain is derived from an IgG1 or IgG4. (49). The composition of any one of embodiments 46-48, wherein the composition comprises a structure of the formula:

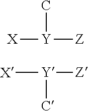

wherein: Y is the first CH2 and CH3 domains; Y′ is the second CH2 and CH3 domains; X and X′ are each independently the first binding domain, the second binding domain, a copy of the first binding domain, a copy of the second binding domain, the cytokine, a copy of the cytokine, a masking polypeptide for the cytokine, or absent; Z and Z′ are each independently the first binding domain, the second binding domain, a copy of the first binding domain, a copy of the second binding domain, the cytokine, a copy of the cytokine, a third binding domain targeting PD-1, or a fourth binding domain targeting VEGFA, a masking polypeptide for the cytokine, or absent. C and C′ are each independently the cytokine, a copy of the cytokine, or absent, wherein C and C′, if present, are attached to a side chain of a residue of the Fc domain via a linker; wherein X, Y, and Z and X′, Y′ and Z′ are depicted in an N-terminal to C-terminal direction; and wherein each of X, X′, Z, and Z′ are independently and optionally connected to Y or Y′ via a peptide linker. (50). The composition of embodiment 49, wherein X is the first binding domain; X′ is a copy of the first binding domain, the cytokine, or absent; one of Z or Z′ is the second binding domain and the other is absent, the cytokine, or a copy of the second binding domain; and one of C or C′ is the cytokine and the other is absent, or both C and C′ are absent. (51). The composition of embodiment 50, wherein X′ is the cytokine and C and C′ are both absent. (52). The composition of embodiment 50, wherein X′ is the copy of the first binding domain and one of C or C′ is the cytokine. (53). The composition of embodiment 50, wherein X′ is the copy of the first binding domain, one of Z or Z′ is the second binding domain and the other is the cytokine, and both C and C′ are absent. (54). The composition of any one of embodiments 50-52, wherein: Z is the second binding domain and Z′ is a copy of the second binding domain; Z is the second binding domain and Z′ is absent; or Z is absent and Z′ is the second binding domain. (55). The composition of embodiment 49, wherein X is the second binding domain, X′ is a copy of the second binding domain, the cytokine, or absent; one of Z or Z′ is the first binding domain and the other is absent, the cytokine, or a copy of the first binding domain; and one of C or C′ is the cytokine and the other is absent, or both C and C′ are absent. (56). The composition of embodiment 55, wherein X′ is the cytokine and C and C′ are both absent. (57). The composition of embodiment 55, wherein X′ is a copy of the second binding domain one of C or C′ is the cytokine. (58). The composition of embodiment 54, wherein X′ is the copy of the second binding domain, one of Z or Z′ is the second binding domain and the other is the cytokine, and both C and C′ are absent. (59). The composition of any one of embodiments 55-57, wherein: Z is the first binding domain and Z′ is a copy of the first binding domain; Z is the first binding domain and Z′ is absent; or Z is absent and Z′ is the first binding domain. (60). The composition of embodiment 59, wherein X is the first binding domain, X′ is the second binding domain, one of C or C′ is the cytokine and the other is absent, Z is absent or the third binding domain, and Z′ is absent or the fourth binding domain. (61). The composition of embodiment 60, wherein: Z is the third binding domain and Z′ is absent; Z is absent and Z′ is the fourth binding domain; or both Z and Z′ are absent. (62). The composition of embodiment 59, wherein X is the first binding domain, X′ is the second binding domain, one of Z or Z′ is the cytokine and the other is the masking polypeptide for the cytokine, and both C and C′ are absent. (63). The composition of embodiment 49 or 62, wherein the masking polypeptide for the cytokine is attached to the Y or Y′ to which it is connected via a cleavable peptide linker. (64). The composition of any one of embodiments 49, 60, or 61, wherein if the third binding domain is present, the first binding domain comprises a Fab and the third binding domain comprises an scFv, and wherein the Fab and the scFv comprise the same VH and VL. (65). The composition of any one of embodiments 49 or 60, or 61, wherein if the fourth binding domain is present, the second binding domain comprises a Fab and the fourth binding domain comprises an scFv, and wherein the Fab and the scFv comprise the same VH and VL. (66). The composition of any one of embodiments 49-65, wherein X is one of the first or second binding domains and is a Fab, VHH, or scFv. (67). The composition of embodiment 66, wherein X is one of the first or second binding domains and is a Fab. (68). The composition of embodiment 66 or 67, wherein X′ is a copy of X. (69). The composition of any one of embodiments 66-68, wherein one Z or Z′ is one of the first or second binding domains and is an scFv or a VHH, wherein if X is the first binding domain then Z or Z′ is the second binding domain and if X is the second binding domain then Z or Z′ is the first binding domain. (70). The composition of embodiment 49, wherein: X is a Fab and the first binding domain, X′ is a copy of the first binding domain, Z is an scFv or VHH and is the second binding domain, Z′ is a copy of the second binding domain, C is the cytokine, and C′ is absent; or X is a Fab and the second binding domain, X′ is a copy of the second binding domain, Z is an scFv or VHH and is the first binding domain, Z′ is a copy of the first binding domain, C is the cytokine, and C′ is absent; or X is a Fab and is the first binding domain, X′ is a Fab and is the second binding domain, Z and Z′ are absent, and C or C′ is the cytokine and the other is absent; X is a Fab and is the first binding domain, X′ is a Fab and is the second binding domain; Z is an scFv or VHH and is the third binding domain, Z′ is absent, and C or C′ is the cytokine and the other is absent; or X is a Fab and is the second binding domain, X′ is a Fab and is the first binding domain, Z is an scFv or VHH and is the fourth binding domain, Z′ is absent, and C or C′ is the cytokine and the other is absent; or X is a Fab and is the first binding domain, X′ is the cytokine, Z is an scFv or VHH and is the second binding domain, Z′ is a copy of the second binding domain, and C and C′ are both absent; or X is a Fab and is the second binding domain, X′ is the cytokine, Z is an scFv or VHH and is the first binding domain, Z′ is a copy of the first binding domain, and C and C′ are both absent; or X is a Fab and is the first binding domain, X′ is an scFv and is the second binding domain; Z and Z′ are both absent, and C or C′ is the cytokine and the other is absent; or X is a Fab and is the second binding domain, X′ is an scFv and is the first binding domain, Z and Z′ are both absent, and C or C′ is the cytokine and the other is absent; or X is a Fab and is the first binding domain, X′ is Fab or ScFv and is the second binding domain, one of Z or Z′ is the cytokine and the other is the masking polypeptide for the cytokine, and C and C′ are both absent; or X is a Fab and is the second binding domain, X′ is a Fab or scFv and is the first binding domain, one of Z or Z′ is the cytokine and the other is the masking polypeptide for the cytokine, and C and C′ are both absent. (71). The composition of any one of embodiments 1-70, wherein the composition comprises only one cytokine. (72). One or more polynucleotides encoding the composition of any one of the preceding embodiments, or a portion thereof. (73). A host cell comprising the composition of any one of embodiments 1-71 or the one or more polynucleotides of embodiment 72. (74). A pharmaceutical composition comprising the composition of any one of embodiments 1-(71, and a pharmaceutically acceptable carrier or excipient. (75). A method of treating cancer in a subject in need thereof, comprising administering to the subject at therapeutically effective amount of a composition of any one of embodiments 1-73 or a pharmaceutical composition of embodiment 74. (76). The immunocytokine composition of any one of embodiments 1-71, wherein the format of the binding domains is as depicted in any one of FIGS. 2A-2D, 3A-3D, 4A-4C, or 5A-5B.

Some additional numbered examples of embodiments follow. (1). A multifunctional immunocytokine composition, comprising: a) a first binding domain targeting programmed cell death protein 1 (PD-1); b) a second binding domain targeting vascular endothelial growth factor A (VEGFA); and c) a cytokine, wherein each of the first binding domain, the second binding domain, and the cytokine are in covalent association. (2). The composition of embodiment 1, wherein the cytokine is selected from an interleukin, a TNF family cytokine, an interferon, a TGF-b family cytokine, and a chemokine. (3). The composition of embodiment 1 or 2, wherein the cytokine is an IL-2 polypeptide. (4). The composition of embodiment 3, wherein the IL-2 polypeptide comprises an amino acid sequence having at least 80%, 85%, 90%, 95%, 96%, 97%, 98%, 99% or 100% a sequence identity to wild type IL-2 (SEQ ID NO: 701). (5). The composition of embodiment 3 or 4, wherein the IL-2 polypeptide enhanced selectivity for the IL-2 receptor beta subunit over the IL-2 receptor alpha subunit compared to the IL-2 of SEQ ID NO: 701. (6). The composition of any one of embodiments 3-5, wherein the IL-2 polypeptide has reduced affinity for the IL-2 receptor beta subunit compared to the IL-2 of SEQ ID NO: 701. (7). The composition of any one of embodiments 3-6, wherein the IL-2 polypeptide exhibits a reduced ability to signal through the IL-2 receptor beta/gamma complex compared to the IL-2 of SEQ ID NO: 701. (8). The composition of any one of embodiments 3-7, wherein the IL-2 polypeptide comprises at least one polymer covalently attached to a residue selected from residues 35, 37, 38, 41, 42, 43, 44, 45, 60, 61, 62, 64, 65, 68, 69, 71, 72, 104, 105, and 107, wherein residue position numbering is based on SEQ ID NO: 701 as a reference sequence, optionally wherein the polymer forms part of a linker which forms the covalent association with the other portions of the composition. (9). The composition of embodiment 8, wherein, the polymer comprises polyethylene glycol. (10). The composition of embodiment 8 or 9, wherein the IL-2 polypeptide comprises polymers attached at residues 42 and 45. (11). The composition of any one of embodiments 8-10, wherein the IL-2 polypeptide comprises a polymer at residue 42 which forms part of the linker which forms the covalent association with the other portions of the composition. (12). The composition of any one of embodiments 3-11, wherein the IL-2 polypeptide comprises an amino acid sequence having at least about 80%, at least about 85%, at least about 90%, or at least about 95% sequence identity to SEQ ID NO: 703, or wherein the IL-2 polypeptide is one described in the subsection titled “Beta-gamma competent IL-2 polypeptides with detuned IL-2 receptor alpha binding”. (13). The composition of embodiment 3 or 4, wherein the IL-2 polypeptide retains the ability to bind to the IL-2 receptor alpha subunit and exhibits a substantially diminished ability to bind to the IL-2 receptor beta or gamma subunits relative to SEQ ID NO: 701 (e.g., such as an IL-2 polypeptide of SEQ ID NO: 775); or wherein the IL-2 polypeptide comprises an amino acid sequence set forth in SEQ ID NO: 775; or wherein the IL-2 polypeptide is one described in the subsection titled “IL-2 Polypeptides Which Selectively Bind to IL-2 Receptor Alpha Subunit”. (14). The composition of embodiment 13, wherein the IL-2 polypeptide comprises one or more of the following modifications: one or more substitutions selected from T3A, Y31H, K35R, Q74P, N88D, N88R, S130R; and/or a substitution of the B′C′ loop of the IL-2 polypeptide with the IL-15B′C′ loop. (15). The composition of embodiment 13, wherein the IL-2 polypeptide comprises the sequence of SEQ ID NO: 706. (16). The composition of any one of embodiments 3-15, wherein the IL-2 polypeptide comprises an amino acid sequence set forth in Table 3. (17). The composition of any one of embodiments 3-16, wherein the IL-2 polypeptide is in covalent association with the other portions of the composition via a linker attached to a side chain of an amino acid residue of the IL-2 polypeptide, or to the N-terminal amine of the IL-2 polypeptide. (18). The composition of embodiment 17, wherein the linker is attached to residue 35, 37, 38, 41, 42, 43, 44, 45, 60, 61, 62, 64, 65, 68, 69, 71, 72, 104, 105, and 107 of the IL-2 polypeptide, wherein residue position numbering is based on SEQ ID NO: 701 as a reference sequence. (19). The composition of embodiment 18, wherein the linker is attached to residue 42 of the IL-2 polypeptide or the N-terminus of the IL-2 polypeptide. (20). The composition of any one of embodiments 17-19, wherein the linker is attached to an Fc region of the composition. (21). The composition of embodiment 20, wherein the linker is attached to the Fc region at a position of a K246 amino acid residue, a K248 amino acid residue, a K288 amino acid residue, a K290 amino acid residue, or a K317 amino acid residue of the Fc region (EU numbering). (22). The composition of embodiment 21, wherein the linker is attached to the K248 residue. (23). The composition of any one of embodiments 3-17, wherein the IL-2 polypeptide is in covalent association via a fusion of the IL-2 polypeptide to the portion of the composition to which it is attached. (24). The composition of any one of embodiments 1-23, wherein the first binding domain targeting programmed cell death protein 1 (PD-1) is capable of disrupting the interaction of PD-1 with programmed cell death ligand 1 (PD-L1). (25). The composition of any one of embodiments 1-24, wherein the first binding domain is an antigen binding fragment derived from an antibody. (26). The composition of any one of embodiments 1-25, wherein the first binding domain comprises a heavy chain variable domain (VH) comprising a heavy chain first complementary determining region (VH CDR1), a heavy chain second complementary determining region (VH CDR2), and a heavy chain third complementary determining region (VH CDR3). (27). The composition of embodiment 26, wherein the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. (28). The composition of embodiment 26 or 27, wherein the VH comprises a set of VH CDR1, VH CDR2, and VH CDR3 derived from an antibody in Table 1A or 1B. (29). The composition of any one of embodiments 26-28, wherein the VH comprise an amino acid sequence of a VH set forth in Table 1A or 1B. (30). The composition of any one of embodiments 26-29, wherein the first binding domain is a VHH. (31). The composition of embodiment 30, wherein the first binding domain comprises: a) a VH CDR1 sequence of SEQ ID NO: 18; a VH CDR2 sequence of SEQ ID NO: 19, and a VH CDR3 sequence of SEQ ID NO: 20; or b) a VH CDR1 sequence of SEQ ID NO: 288, a VH CDR2 sequence of SEQ ID NO: 289, and a VH CDR3 sequence of SEQ ID NO: 290. (32). The composition of embodiment 30 or 31, wherein the VHH comprises an amino acid sequence having at least 80%, 85%, 90%, 95%, 96%, 97%, 98%, 99%, or 100% identity to any one of SEQ ID NOs: 17 or 287. (33). The composition of any one of embodiments 26-29, the VH comprises a) a VH CDR1 sequence of SEQ ID NO: 80, a VH CDR2 sequence of SEQ ID NO: 81, and a VH CDR3 sequence of SEQ ID NO: 82; b) a VH CDR1 sequence of SEQ ID NO:86, a VH CDR2 sequence of SEQ ID NO: 87, and a VH CDR3 sequence of SEQ ID NO: 88; or c) a VH CDR1 sequence of SEQ ID NO: 113, a VH CDR2 sequence of SEQ ID NO: 114, and a VH CDR3 sequence of SEQ ID NO: 115. (34). The composition of any one of embodiments 26-29 or 33, wherein the VH comprises an amino acid sequence having at least 80%, 85%, 90%, 95%, 96%, 97%, 98%, 99%, or 100% identity to any one of SEQ ID NOs: 48, 50, or 76 (35). The composition of any one of embodiments 1-29 or 33-34 or, wherein the first binding domain comprises a light chain variable domain (VL) comprising a light chain first complementary determining region (VL CDR1), a light chain second complementary determining region (VL CDR2), and a light chain third complementary determining region (VL CDR3). (36). The composition of embodiment 35, wherein the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the first binding domain is comprised. (37). The composition of embodiment 35 or 36, wherein the VL comprises a set of VL CDR1, VL CDR2, and VL CDR3 derived from an antibody in Table 1A or 1B. (38). The composition of anyone of embodiments 35-37, wherein the VL comprises: a) a VL CDR1 sequence of SEQ ID NO: 83, a VL CDR2 sequence of SEQ ID NO: 84, and a VL CDR3 sequence of SEQ ID NO: 85; b) a VL CDR1 sequence of SEQ ID NO: 89, a VL CDR2 sequence of SEQ ID NO: 90), and a VL CDR3 sequence of SEQ ID NO: 91; or c) a VL CDR1 sequence of SEQ ID NO: 116, a VL CDR2 sequence of SEQ ID NO: 117, and a VL CDR3 sequence of SEQ ID NO: 118. (39). The composition of any one of embodiments 35-38, wherein the VL comprises an amino acid sequence having at least 80%, 85%, 90%, 95%, 96%, 97%, 98%, 99%, or 100% identity to any one of SEQ ID NOs: 49, 51, or 77. (40). The composition of any one of embodiments 1-29 or 33-39, wherein the first binding domain comprises: a) a VH having a VH CDR1 sequence of SEQ ID NO: 80, a VH CDR2 sequence of SEQ ID NO: 81, and a VH CDR3 sequence of SEQ ID NO: 82, and a VL having a VL CDR1 sequence of SEQ ID NO: 83, a VL CDR2 sequence of SEQ ID NO: 84, and a VL CDR3 sequence of SEQ ID NO: 85; or b) a VH having a VH CDR1 sequence of SEQ ID NO: 86, a VH CDR2 sequence of SEQ ID NO: 87, and a VH CDR3 sequence of SEQ ID NO: 88, and a VL having a VL CDR1 sequence of SEQ ID NO: 89, a VL CDR2 sequence of SEQ ID NO: 90, and a VL CDR3 sequence of SEQ ID NO: 91; or c) a VH having a VH CDR1 sequence of SEQ ID NO: 113, a VH CDR2 sequence of SEQ ID NO: 114, and a VH CDR3 sequence of SEQ ID NO: 115, and a VL having a VL CDR1 sequence of SEQ ID NO: 116, a VL CDR2 sequence of SEQ ID NO: 117, and a VL CDR3 sequence of SEQ ID NO: 118. (41). The composition of anyone of embodiments 1-29 or 33-40, wherein the first binding domain comprises: a) a VH comprising an amino acid sequence amino acid sequence having at least 80%, 85%, 90%, 95%, 96%, 97%, 98%, 99%, or 100% identity to SEQ ID NO: 48 and a VL comprising an amino acid sequence having at least 80%, 85%, 90%, 95%, 96%, 97%, 98%, 99%, or 100% identity to SEQ ID NO: 49; b) a VH comprising an amino acid sequence amino acid sequence having at least 80%, 85%, 90%, 95%, 96%, 97%, 98%, 99%, or 100% identity to SEQ ID NO: 50 and a VL comprising an amino acid sequence having at least 80%, 85%, 90%, 95%, 96%, 97%, 98%, 99%, or 100% identity to SEQ ID NO: 51; c) a VH comprising an amino acid sequence amino acid sequence having at least 80%, 85%, 90%, 95%, 96%, 97%, 98%, 99%, or 100% identity to SEQ ID NO: 76 and a VL comprising an amino acid sequence having at least 80%, 85%, 90%, 95%, 96%, 97%, 98%, 99%, or 100% identity to SEQ ID NO: 77; d) a VH comprising an amino acid sequence amino acid sequence having at least 80%, 85%, 90%, 95%, 96%, 97%, 98%, 99%, or 100% identity to SEQ ID NO: 9 and a VL comprising an amino acid sequence having at least 80%, 85%, 90%, 95%, 96%, 97%, 98%, 99%, or 100% identity to SEQ ID NO: 10; or e) a VH comprising an amino acid sequence amino acid sequence having at least 80%, 85%, 90%, 95%, 96%, 97%, 98%, 99%, or 100% identity to SEQ ID NO: 11 and a VL comprising an amino acid sequence having at least 80%, 85%, 90%, 95%, 96%, 97%, 98%, 99%, or 100% identity to SEQ ID NO: 12. (42). The composition of any one of embodiments 1-29 or 33-41, wherein the first binding domain is an scFv. (43). The composition of any one of embodiments 1-29 or 33-41, wherein the first binding domain is a Fab. (44). The composition of any one of embodiments 1-43, wherein the second binding domain targeting VEGFA is capable of disrupting the interaction of VEGFA with one or more of its receptors. (45). The composition of any one of embodiments 1-44, wherein the second binding domain is comprised in an antigen binding fragment derived from an antibody. (46). The composition of any one of embodiments 1-45, wherein the second binding domain comprises a heavy chain variable domain (VH) comprising a heavy chain first complementary determining region (VH CDR1), a heavy chain second complementary determining region (VH CDR2), and a heavy chain third complementary determining region (VH CDR3). (47). The composition of embodiment 46, wherein the VH is comprised in a Fab, a Fab′, F(ab′)₂, a bispecific F(ab′)₂, a variable fragment (Fv), a single chain variable fragment (scFv), a bispecific scFv, disulfide stabilized Fv (dsFv), a minibody, a diabody, a bispecific diabody, triabody, a tetrabody, a maxibody, a camelid, a VHH, a Fab-Fc, a scFv-Fc, or a bispecific antibody. (48). The composition of embodiment 46 or 47, wherein the VH comprises a set of VH CDR1, VH CDR2, and VH CDR3 derived from an antibody in Table 2A or 2B. (49). The composition of any one of embodiments 46-48, wherein the VH comprise an amino acid sequence of a VH set forth in Table 2A or 2B. (50). The composition of any one of embodiments 46-49, wherein the VH comprises a VH CDR1 having a sequence SEQ ID NO: 123, a VH CDR2 having a sequence SEQ ID NO: 124, and a VH CDR3 having a sequence SEQ ID NO: 125. (51). The composition of any one of embodiments 46-50, wherein the VH comprises an amino acid sequence having at least 80%, 85%, 90%, 95%, 96%, 97%, 98%, 99%, or 100% identity to SEQ ID NO: 122. (52). The composition of any one of embodiments 1-51, wherein the second binding domain comprises a light chain variable domain (VL) comprising a light chain first complementary determining region (VL CDR1), a light chain second complementary determining region (VL CDR2), and a light chain third complementary determining region (VL CDR3). (53). The composition of embodiment 52, wherein the VL is comprised in the Fab, Fab′, F(ab′)₂, bispecific F(ab′)₂, variable fragment (Fv), single chain variable fragment (scFv), bispecific scFv, disulfide stabilized Fv (dsFv), minibody, diabody, bispecific diabody, triabody, tetrabody, maxibody, Fab-Fc, scFv-Fc, or bispecific antibody in which the VH of the second binding domain is comprised. (54). The composition of embodiment 52 or 53, wherein the VL comprises a set of VL CDR1, VL CDR2, and VL CDR3 derived from an antibody in Table 2A or 2B. (55). The composition of any one of embodiments 52-54, wherein the VL comprises a VL CDR1 having a sequence SEQ ID NO: 128, a VL CDR2 having a sequence SEQ ID NO: 129, and a VL CDR3 having a sequence SEQ ID NO: 130. (56). The composition of any one of embodiments 52-55, wherein the VL comprises an amino acid sequence having at least 80%, 85%, 90%, 95%, 96%, 97%, 98%, 99%, or 100% identity to SEQ ID NO: 127. (57). The composition of any one of embodiments 1-56, wherein the second binding domain comprises a VH having a VH CDR1 having a sequence SEQ ID NO: 123, a VH CDR2 having a sequence SEQ ID NO: 124, and a VH CDR3 having a sequence SEQ ID NO: 125, and a VL having a VL CDR1 having a sequence SEQ ID NO: 128, a VL CDR2 having a sequence SEQ ID NO: 129, and a VL CDR3 having a sequence SEQ ID NO: 130. (58). The composition of any one of embodiments 1-57, wherein the second binding domain comprises a VH comprising an amino acid sequence having at least 80%, 85%, 90%, 95%, 96%, 97%, 98%, 99%, or 100% sequence identity to SEQ ID NO: 122 and a VL comprising an amino acid sequence having at least 80%, 85%, 90%, 95%, 96%, 97%, 98%, 99%, or 100% sequence identity to SEQ ID NO: 127 (59). The composition of any one of embodiments 1-58, wherein the second binding domain is an scFv. (60). The composition of any one of embodiments 1-58, wherein the second binding domain is a Fab. (61). The composition of any one of embodiments 1-49, wherein the second binding domain is a single domain antibody. (62). The composition of embodiment 61, wherein the second binding domain is a single domain heavy chain antibody (VHH). (63). The composition of embodiment 61 or 62, wherein the second binding domain comprises: a) a VH CDR1 sequence of SEQ ID NO: 202, a VH CDR2 sequence of SEQ ID NO: 203, and a VH CDR3 sequence of SEQ ID NO: 204; b) a VH CDR1 sequence of SEQ ID NO: 206, a VH CDR2 sequence of SEQ ID NO: 207, and a VH CDR3 sequence of SEQ ID NO: 208; c) a VH CDR1 sequence of SEQ ID NO: 210, a VH CDR2 sequence of SEQ ID NO: 211, and a VH CDR3 sequence of SEQ ID NO: 212; d) a VH CDR1 sequence of SEQ ID NO: 214, a VH CDR2 sequence of SEQ ID NO: 215, and a VH CDR3 sequence of SEQ ID NO: 216; or e) a VH CDR1 having the sequence SEQ ID NO: 218, a VH CDR2 having the sequence SEQ ID NO: 219 or the sequence of SEQ ID NO: 279, and a VH CDR3 having the sequence SEQ ID NO: 220. (64). The composition of any one of embodiments 61-63, wherein the second binding domain comprises an amino acid sequence having at least 80%, 85%, 90%, 95%, 96%, 97%, 98%, 99% or 100% identity to any one of SEQ ID NOs: 201, 205, 209, 213, 217, 221, or 280-284. (65). The composition of embodiment 61, wherein the second binding domain comprises a light chain single domain antibody. (66). The composition of embodiment 61 or 65, wherein the second binding domain comprises a VL CDR1 having the sequence SEQ ID NO: 223, a VL CDR2 having the sequence SEQ ID NO: 224, and a VL CDR3 having the sequence SEQ ID NO: 225. (67). The composition of any one of embodiments 61, 65, or 66, wherein the second binding domain comprises an amino acid sequence having at least 80%, 85%, 90%, 95%, 96%, 97%, 98%, 99% or 100% identity to SEQ ID NO: 222. (68). The composition of any one of embodiments 1-44, wherein the second binding domain is an anti-VEGFA anticalin. (69). The composition of any one of embodiments 1-44 or 68, wherein the second binding domain comprises an amino acid sequence having at least 80%, 85%, 90%, 95&, 96%, 97%, 98%, 99% or 100% identity to SEQ ID NO: 226. (70). The composition of any one of embodiments 1-69, wherein the composition comprises an Fc domain comprising first CH2 and CH3 domains on a first polypeptide chain and second CH2 and CH3 domains on a second polypeptide chain. (71). The composition of embodiment 70, wherein the Fc domain is derived from an IgG. (72). The composition of embodiment 71, wherein the Fc domain is derived from an IgG1 or IgG4. (73). The composition of any one of embodiments 70-72, wherein the composition comprises a structure of the formula:

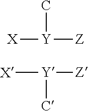

wherein: Y is the first CH2 and CH3 domains; Y′ is the second CH2 and CH3 domains; X and X′ are each independently the first binding domain, the second binding domain, a copy of the first binding domain, a copy of the second binding domain, the cytokine, a copy of the cytokine, a masking polypeptide for the cytokine, or absent; Z and Z′ are each independently the first binding domain, the second binding domain, a copy of the first binding domain, a copy of the second binding domain, the cytokine, a copy of the cytokine, a third binding domain targeting PD-1, or a fourth binding domain targeting VEGFA, a masking polypeptide for the cytokine, or absent. C and C′ are each independently the cytokine, a copy of the cytokine, or absent, wherein C and C′, if present, are attached to a side chain of a residue of the Fc domain via a linker; wherein X, Y, and Z and X′, Y′ and Z′ are depicted in an N-terminal to C-terminal direction; and wherein each of X, X′, Z, and Z′ are independently and optionally connected to Y or Y′ via a peptide linker. (74). The composition of embodiment 73, wherein X is the first binding domain; X′ is a copy of the first binding domain, the cytokine, or absent; one of Z or Z′ is the second binding domain and the other is absent, the cytokine, or a copy of the second binding domain; and one of C or C′ is the cytokine and the other is absent, or both C and C′ are absent. (75). The composition of embodiment 74, wherein X′ is the cytokine and C and C′ are both absent. (76). The composition of embodiment 74, wherein X′ is the copy of the first binding domain and one of C or C′ is the cytokine. (77). The composition of embodiment 74, wherein X′ is the copy of the first binding domain, one of Z or Z′ is the second binding domain and the other is the cytokine, and both C and C′ are absent. (78). The composition of any one of embodiments 74-76, wherein: Z is the second binding domain and Z′ is a copy of the second binding domain; Z is the second binding domain and Z′ is absent; or Z is absent and Z′ is the second binding domain. (79). The composition of embodiment 73, wherein X is the second binding domain, X′ is a copy of the second binding domain, the cytokine, or absent; one of Z or Z′ is the first binding domain and the other is absent, the cytokine, or a copy of the first binding domain; and one of C or C′ is the cytokine and the other is absent, or both C and C′ are absent. (80). The composition of embodiment 79, wherein X′ is the cytokine and C and C′ are both absent. (81). The composition of embodiment 79, wherein X′ is a copy of the second binding domain one of C or C′ is the cytokine. (82). The composition of embodiment 78, wherein X′ is the copy of the second binding domain, one of Z or Z′ is the second binding domain and the other is the cytokine, and both C and C′ are absent. (83). The composition of any one of embodiments 79-81, wherein: Z is the first binding domain and Z′ is a copy of the first binding domain; Z is the first binding domain and Z′ is absent; or Z is absent and Z′ is the first binding domain. (84). The composition of embodiment 73, wherein X is the first binding domain, X′ is the second binding domain, one of C or C′ is the cytokine and the other is absent, Z is absent or the third binding domain, and Z′ is absent or the fourth binding domain. (85). The composition of embodiment 84, wherein: Z is the third binding domain and Z′ is absent; Z is absent and Z′ is the fourth binding domain; or both Z and Z′ are absent. (86). The composition of embodiment 73, wherein X is the first binding domain, X′ is the second binding domain, one of Z or Z′ is the cytokine and the other is the masking polypeptide for the cytokine, and both C and C′ are absent. (87). The composition of embodiment 73 or 86, wherein the masking polypeptide for the cytokine is attached to the Y or Y′ to which it is connected via a cleavable peptide linker. (88). The composition of any one of embodiments 73, 86, or 87, wherein if the third binding domain is present, the first binding domain comprises a Fab and the third binding domain comprises an scFv, and wherein the Fab and the scFv comprise the same VH and VL. (89). The composition of any one of embodiments 73, 86, or 87, wherein if the fourth binding domain is present, the second binding domain comprises a Fab and the fourth binding domain comprises an scFv, and wherein the Fab and the scFv comprise the same VH and VL. (90). The composition of any one of embodiments 73-89, wherein X is one of the first or second binding domains and is a Fab, VHH, or scFv. (91). The composition of embodiment 90, wherein X is one of the first or second binding domains and is a Fab. (92). The composition of embodiment 90 or 91, wherein X′ is a copy of X. (93). The composition of any one of embodiments 90-92, wherein one Z or Z′ is one of the first or second binding domains and is an scFv or a VHH, wherein if X is the first binding domain then Z or Z′ is the second binding domain and if X is the second binding domain then Z (or Z′ is the first binding domain. 94). The composition of embodiment 73, wherein: X is a Fab and the first binding domain, X′ is a copy of the first binding domain, Z is an scFv or VHH and is the second binding domain, Z′ is a copy of the second binding domain, C is the cytokine, and C′ is absent; or X is a Fab and the second binding domain, X′ is a copy of the second binding domain, Z is an scFv or VHH and is the first binding domain, Z′ is a copy of the first binding domain, C is the cytokine, and C′ is absent; or X is a Fab and is the first binding domain, X′ is a Fab and is the second binding domain, Z and Z′ are absent, and C or C′ is the cytokine and the other is absent; X is a Fab and is the first binding domain, X′ is a Fab and is the second binding domain; Z is an scFv or VHH and is the third binding domain, Z′ is absent, and C or C′ is the cytokine and the other is absent; or X is a Fab and is the second binding domain, X′ is a Fab and is the first binding domain, Z is an scFv or VHH and is the fourth binding domain, Z′ is absent, and C or C′ is the cytokine and the other is absent; or X is a Fab and is the first binding domain, X′ is the cytokine, Z is an scFv or VHH and is the second binding domain, Z′ is a copy of the second binding domain, and C and C′ are both absent; or X is a Fab and is the second binding domain, X′ is the cytokine, Z is an scFv or VHH and is the first binding domain, Z′ is a copy of the first binding domain, and C and C′ are both absent; or X is a Fab and is the first binding domain, X′ is an scFv and is the second binding domain; Z and Z′ are both absent, and C or C′ is the cytokine and the other is absent; or X is a Fab and is the second binding domain, X′ is an scFv and is the first binding domain, Z and Z′ are both absent, and C or C′ is the cytokine and the other is absent; or X is a Fab and is the first binding domain, X′ is Fab or ScFv and is the second binding domain, one of Z or Z′ is the cytokine and the other is the masking polypeptide for the cytokine, and C and C′ are both absent; or X is a Fab and is the second binding domain, X′ is a Fab or scFv and is the first binding domain, one of Z or Z′ is the cytokine and the other is the masking polypeptide for the cytokine, and C and C′ are both absent. (95). The composition of any one of embodiments 1-94, wherein the composition comprises only one cytokine. (96). One or more polynucleotides encoding the composition of any one of the preceding embodiments, or a portion thereof. (97). A host cell comprising the composition of any one of embodiments 1-95 or the one or more polynucleotides of embodiment 96. (98). A pharmaceutical composition comprising the composition of any one of embodiments 1-95, and a pharmaceutically acceptable carrier or excipient. (99). A method of treating cancer in a subject in need thereof, comprising administering to the subject at therapeutically effective amount of a composition of any one of embodiments 1-95 or a pharmaceutical composition of embodiment 98.

What is claimed is:

1. A multifunctional immunocytokine composition, comprising:
a) a first binding domain targeting programmed cell death protein 1 (PD-1);
b) a second binding domain targeting vascular endothelial growth factor A (VEGFA); and
c) an IL-2 polypeptide,
wherein each of the first binding domain, the second binding domain, and the IL-2 polypeptide are in covalent association.

2. The composition of claim 1, wherein the IL-2 polypeptide comprises an amino acid sequence having at least 80%, 85%, 90%, 95%, 96%, 97%, 98%, 99% or 100% a sequence identity to wild type IL-2 (SEQ ID NO: 701).

3. The composition of claim 1, wherein the IL-2 polypeptide comprises a modified B′C′ loop region, wherein the modified B′C′ loop region comprises a deletion of one or more amino acids of the B′C′ loop region between amino acids 73 and 84 of the IL-2 polypeptide and insertion of an exogenous peptide into the B′C′ loop region, wherein the inserted peptide comprises the sequence GDGSIN (SE ID NO: 700), wherein residue position numbering is based on SEQ ID NO: 701 as a reference sequence.

4. The composition of claim 3, wherein the deletion of one or more amino acids of the B′C′ loop region comprises a deletion of each of amino acids 74-83 of the IL-2 polypeptide.

5. The composition of claim 1, wherein the IL-2 polypeptide comprises an N88D substitution

6. The composition of claim 5, wherein the IL-2 polypeptide comprises any one of the following sets of substitutions:
Q126T;
I129K;
I129A, E15S, T123A;
E15D;
L12A, L19A, E15S;
L12Y, L19D;
L12A, L19A; or
L19D.

7. The composition of claim 6, wherein the IL-2 polypeptide comprises an E15D or an L19D substitution.

8. The composition of claim 6, wherein the IL-2 polypeptide comprises a T3A substitution.

9. The composition of claim 6, wherein the IL-2 polypeptide comprises a C125S substitution.

10. The composition of claim 1, wherein the IL-2 polypeptide comprises the amino acid sequence set forth in SEQ ID NO: 751, SEQ ID NO: 753, SEQ ID NO: 754, or SEQ ID NO: 758.

11. The composition of claim 1, wherein the first binding domain is a VHH comprising:
a VH CDR1 sequence of SEQ ID NO: 510, a VH CDR2 sequence of SEQ ID NO: 511, and a VHCDR3 sequence of SEQ ID NO: 512.

12. The composition of claim 11, wherein the VHH comprises an amino acid sequence having at least 80% identity to any one of SEQ ID NOs: 509, 693, 694, 695, 696, or 698.

13. The composition of claim 1, wherein the first binding domain comprises:
a) a VH having a VH CDR1 sequence of NYYMY (SEQ ID NO: 80), a VH CDR2 sequence of GINPSNGGTNFNEKFKN (SEQ ID NO: 81), and a VH CDR3 sequence of RDYRFDMGFDY (SEQ ID NO: 82), and a VL having a VL CDR1 sequence of RASKGVSTSGYSYLH (SEQ ID NO: 83), a VL CDR2 sequence of LASYLES (SEQ ID NO: 84), and a VL CDR3 sequence of QHSRDLPLT (SEQ ID NO: 85); or
b) a VH having a VH CDR1 sequence of NSGMH (SEQ ID NO: 86), a VH CDR2 sequence of VIWYDGSKRYYADSVKG (SEQ ID NO: 87), and a VH CDR3 sequence of NDDY (SEQ ID NO: 88), and a VL having a VL CDR1 sequence of RASQSVSSYLA (SEQ ID NO: 89), a VL CDR2 sequence of DASNRAT (SEQ ID NO: 90), and a VL CDR3 sequence of QQSSNWPRT (SEQ ID NO: 91); or
c) a VH having a VH CDR1 sequence of GYTFTSYYMY (SEQ ID NO: 113), a VH CDR2 sequence of GVNPSNGGTNFNEKFKS (SEQ ID NO: 114), and a VH CDR3 sequence of RDYRYDMGFDY (SEQ ID NO: 115), and a VL having a VL CDR1 sequence of RASKGVSTSGYSYLH (SEQ ID NO: 83), a VL CDR2 sequence of LASYLE (SEQ ID NO: 117), and a VL CDR3 sequence of QHSRELPLT (SEQ ID NO: 118).

14. The composition of claim 13, wherein the first binding domain comprises:
a) a VH comprising an amino acid sequence amino acid sequence having at least 80%, 85%, 90%, 95%, 96%, 97%, 98%, 99%, or 100% identity to SEQ ID NO: 48 and a VL comprising an amino acid sequence having at least 80%, 85%, 90%, 95%, 96%, 97%, 98%, 99%, or 100% identity to SEQ ID NO: 49;
b) a VH comprising an amino acid sequence amino acid sequence having at least 80%, 85%, 90%, 95%, 96%, 97%, 98%, 99%, or 100% identity to SEQ ID NO: 50 and a VL comprising an amino acid sequence having at least 80%, 85%, 90%, 95%, 96%, 97%, 98%, 99%, or 100% identity to SEQ ID NO: 51; or
c) a VH comprising an amino acid sequence amino acid sequence having at least 80%, 85%, 90%, 95%, 96%, 97%, 98%, 99%, or 100% identity to SEQ ID NO: 76 and a VL comprising an amino acid sequence having at least 80%, 85%, 90%, 95%, 96%, 97%, 98%, 99%, or 100% identity to SEQ ID NO: 77.

15. The composition of any one of claim 14, wherein the first binding domain is an scFv or a Fab.

16. The composition of claim 1, wherein the second binding domain comprises a VH having a VH CDR1 having a sequence GYTFTNYGMN (SEQ ID NO: 123), a VH CDR2 having a sequence WINTYTGEPTYAADFK (SEQ ID NO: 124), and a VH CDR3 having a sequence YPHYYGSSHWYFDV (SEQ ID NO: 125), and a VL having a VL CDR1 having a sequence SASQDISNYLN (SEQ ID NO: 128), a VL CDR2 having a sequence FTSSLHS (SEQ ID NO: 129), and a VL CDR3 having a sequence QQYSTVPWT (SEQ ID NO: 130).

17. The composition of claim 16, wherein the second binding domain comprises a VH comprising an amino acid sequence having at least 80%, 85%, 90%, 95%, 96%, 97%, 98%, 99%, or 100% sequence identity to SEQ ID NO: 122 and a VL comprising an amino acid sequence having at least 80%, 85%, 90%, 95%, 96%, 97%, 98%, 99%, or 100% sequence identity to SEQ ID NO: 127.

18. The composition of claim 16, wherein the second binding domain is an scFv or a Fab.

19. The composition of claim 1, wherein the second binding domain is a single domain heavy chain antibody (VHH) comprising:
a VH CDR1 having the sequence SYSMG (SEQ ID NO: 218), a VH CDR2 having the sequence AISKGGYKYDAVSLEG (SEQ ID NO: 219) or the sequence of SEQ ID NO: 279, and a VH CDR3 having the sequence SRAYGSSRLRLADTYEY (SEQ ID NO: 220).

20. The composition of claim 19, wherein the second binding domain comprises an amino acid sequence having at least 80%, 85%, 90%, 95%, 96%, 97%, 98%, 99% or 100% identity to any one of SEQ ID NOs: 200, 217, 221, 280, or 281.

21. The composition of claim 1, wherein the first binding domain comprises a Fab, and wherein the composition comprises:
a) a first polypeptide chain comprising the VL of the first binding domain;
b) a second polypeptide chain comprising the VH of the first binding domain; and
c) a third polypeptide chain comprising the IL-2 polypeptide.

22. The composition of claim 21, wherein the second polypeptide chain comprises, in an N-terminal to C-terminal direction, the VH of the Fab, the antibody constant region, an optional peptide linker, and the second binding domain, wherein the second binding domain is a VHH.

23. The composition of claim 21, wherein the third polypeptide chain comprises, in an N-terminal to C-terminal direction, the IL-2 polypeptide, an optional peptide linker, and an antibody constant region.

24. The composition of claim 21, wherein the first polypeptide chain, the second polypeptide chain, and the third polypeptide chain comprise, respectively, the amino acid sequences set forth in:
SEQ ID NOs: 47, 163, and 802;
SEQ ID NOs: 47, 163, and 803;
SEQ ID NOs: 47, 164, and 806;
SEQ ID NOs: 47, 164, and 807;
SEQ ID NOs: 47, 169, and 802;
SEQ ID NOs: 47, 169, and 803;
SEQ ID NOs: 47, 172, and 802;
SEQ ID NOs: 47, 172, and 803;
SEQ ID NOs: 47, 163, and 806;
SEQ ID NOs: 47, 163, and 807;
SEQ ID NOs: 47, 169, and 806;
SEQ ID NOs: 47, 169, and 807;
SEQ ID NOs: 47, 171, and 809; or
SEQ ID NOs: 47, 171, and 810.

25. The composition of claim 1, wherein the second binding domain is a Fab having a VH and a VL, wherein the composition comprises a) a first polypeptide chain comprising the VL of the first binding domain; b) a second polypeptide chain comprising the VH of the first binding domain; and c) a third polypeptide chain comprising the IL-2 polypeptide.

26. The composition of claim 25, wherein the second polypeptide chain comprises, in an N-terminal to C-terminal direction, the VH of the Fab, the antibody constant region, an optional peptide linker, and the first binding domain specific for PD-1, wherein the first binding domain is a VHH.

27. The composition of claim 25, wherein the third polypeptide chain comprises, in an N-terminal to C-terminal direction, a VH of a second Fab specific for VEGFA, an antibody constant region, an optional peptide linker, and the IL-2 polypeptide.

28. A method of treating cancer in a subject in need thereof, comprising administering to the subject a therapeutically effective amount of a composition comprising:
a) a first binding domain targeting programmed cell death protein 1 (PD-1);
b) a second binding domain targeting vascular endothelial growth factor A (VEGFA); and
c) a cytokine,
wherein each of the first binding domain, the second binding domain, and the cytokine are in covalent association.

29. An IL-2 polypeptide comprising a modified B′C′ loop region, wherein the modified B′C′ loop region comprises a deletion of one or more amino acids of the B′C′ loop region between amino acids 73 and 84 of the IL-2 polypeptide, wherein residue position numbering is based on SEQ ID NO: 701 as a reference sequence, and an insertion of a peptide comprising the sequence GDGSIN into the deleted portion of the B′C′ loop region.

30. An IL-2 polypeptide comprising the amino acid substitutions K76A and R81S, wherein residue position numbering is based on SEQ ID NO: 1 as a reference sequence.
